(19) United States
(12) Patent Application Publication
(10) Pub. No.: US 2009/0191548 A1
(43) Pub. Date: Jul. 30, 2009

(54) METHODS AND NUCLEIC ACIDS FOR THE ANALYSIS OF GENE EXPRESSION ASSOCIATED WITH TISSUE CLASSIFICATION

(75) Inventors: Kurt Berlin, Stahnsdorf (DE); Stephan Beck, Cambridge (GB); Matthias Burger, Berlin (DE); Rene Cortese, Berlin (DE); Florian Eckhardt, Berlin (DE); Carolina Haefliger, Basel (CH); Joern Lewin, Berlin (DE); Fabian Model, Berlin (DE); Alexander Olek, Berlin (DE)

Correspondence Address: DAVIS WRIGHT TREMAINE, LLP/Seattle, 1201 Third Avenue, Suite 2200, SEATTLE, WA, 98101-3045, US

(73) Assignee: Epigenomics AG, Berlin (DE)

(21) Appl. No.: 12/088,384

(22) PCT Filed: Sep. 28, 2006
(86) PCT No.: PCT/EP2006/009454

§ 371 Date: Jul. 2, 2008

(30) Foreign Application Priority Data
Sep. 29, 2005 (EP) 050213313.3
Dec. 23, 2005 (EP) 05090346.7
Jun. 15, 2006 (EP) 06090110.5

(51) Int. Cl. C12Q 1/68; C07H 21/04

(52) U.S. Cl. 435/6; 536/23.1

(57) ABSTRACT

The present application provides methods and nucleic acids the classification of a biological sample. This is achieved by the analysis of the expression status of at least one of the genes selected from Table 1 as disclosed.

### FIELD OF THE INVENTION

The present invention relates to human DNA sequences that exhibit tissue specific expression patterns. Particular embodiments of the invention provide methods for classifying a biological sample.

### BACKGROUND

Bisulfite modification of DNA is an art-recognized tool used to assess CpG methylation status. 5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil, which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyze individual cells for methylation status, illustrating the utility and sensitivity of the method. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek and Walter, Nat Genet. 1997 17:275-6, 1997), subjected to one or more primer extension reactions (Gonzalgo and Jones, Nucleic Acids Res., 25:2529-31, 1997; WO 95/00669; U.S. Pat. No. 6,251,594) to analyze individual cytosine positions, or treated by enzymatic digestion (Xiong and Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg and Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 97/46705 and WO 95/15373).

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri and Hornsby (Nucl. Acids Res. 24:5058-5059, 1996), or COBRA (Combined Bisulfite Restriction Analysis) (Xiong and Laird, Nucleic Acids Res. 25:2532-2534, 1997).

Preferably, assays such as “MethyLight™” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo and Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), are used alone or in combination with other of these methods.

MethyLight™. The MethyLight™ assay is a high-throughput quantitative methylation assay that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or bisulfite treated DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo and Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or bisulfite treated DNA sequence or CpG island); optimized PCR buffers and deoxynucleotides; gel extraction kit; positive control primers; Ms-SNuPE primers for specific gene; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

MSP. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated DNA specific PCR primers for specific gene (or bisulfite treated DNA sequence or CpG island), optimized PCR buffers and deoxynucleotides, and specific probes.

Prior art of several markers. If not stated otherwise, the following information was received from the Entrez database (http://www.ncbi.nlm.nih.gov/entrez/query.fcgi?db=gene) and the OMIM database (http://www.ncbi.nlm.nih.gov/entrez/query.fcgi?db=OMIM).

Glycoprotein Ib (platelet) beta polypeptide. (GP1BB) Glycoprotein Ib (platelet) beta polypeptide (GP1BB) is subunit of the platelet glycoprotein Ib (GPIb). GPIb is a heterodimeric transmembrane protein consisting of a disulfide-linked 140 kD alpha chain and 22 kD beta chain. It is part of the GPIb-V-IX system that constitutes the receptor for von Willebrand factor (VWF), and mediates platelet adhesion in the arterial circulation. GPIb alpha chain provides the VWF binding site, and GPIb beta contributes to surface expression of the receptor and participates in transmembrane signaling through phosphorylation of its intracellular domain. Mutations in the GPIb beta subunit have been associated with Bernard-Soulier syndrome, velocardiofacial syndrome and giant platelet disorder. The 206 amino acid precursor of GPIb beta is synthesized from a 1.0 kb mRNA expressed in platelets and megakaryocytes. A 411 amino acid protein arising from a longer, unspliced transcript in endothelial cells has been described; however, the authenticity of this product has been questioned. Yet another less abundant GPIb beta mRNA species of 3.5 kb, expressed in nonhematopoietic tissues such as endothelium, brain and heart, was shown to result from inefficient usage of a non-consensus polyA_signal within a separate gene (PNUTL1) located upstream of this gene. In the absence of polyadenylation from its own imperfect site, the PNUTL1 gene uses the consensus polyA_signal of this gene (Entrez database).

Transcription factor AP-2 alpha. Transcription factor AP-2 alpha (TFAP2A, alias AP-2 AP2TF; TFAP2; AP-2alpha) also known as activating enhancer binding protein 2 alpha is a 52-kD retinoic acid-inducible and developmentally regulated activator of transcription that binds to a consensus DNA-binding sequence CCCCAGGC in the SV40 and metallothionein (MIM 156350) promoters (OMIM database). The loss of transcription factor AP-2alpha expression has been shown to associate with tumorigenicity of melanoma cell lines and poor prognosis in primary cutaneous melanoma. Altogether these findings suggest that the gene encoding AP-2alpha (TFAP2A) acts as a tumor suppressor in melanoma. A failure in post-transcriptional processing of AP-2alpha is a possible inactivation mechanism of AP-2alpha in cutaneous melanoma (Karjalainen et al, 2000). It was shown that the induction of AP-2 mRNA is at the level of transcription and is transient, reaching a peak 48-72 hr after the addition of retinoic acid (RA) and declining thereafter, even in the continuous presence of retinoic acid. AP-2-binding site-mediated cAMP and TPA (12-O-tetradecanoyl-phorbol-13-acetate) responses are not regulated at the level of AP-2 expression but, rather, achieved either by post-translational changes in AP-2 or in conjunction with another protein (Luscher et al, 1989). Decreases in AP2 protein were rapidly reversed by insulin administration. There were no changes in AP2 protein in the absence of changes in AP2 mRNA supporting a pretranslational mechanism of regulation (Melki and Abumrad, 1993). There is a significant up-regulation of AP-2gamma expression in breast cancer specimens (P=0.01). There was also a significant correlation between the presence of the AP-2alpha protein and estrogen receptor expression (P=0.018) and between specimens containing both AP-2alpha/AP-2gamma proteins and ERBB-2 expression (P=0.003). Furthermore, we detected an association (P=0.04) between the expression of AP-2gamma and the presence of an additional signal transduction molecule implicated in breast cancer (Turner et al, 1998). Macrophages deficient in AP2 display alterations in inflammatory cytokine production (Linton and Fazio, 2003). Loss of AP-2 results in up-regulation of MCAM/MUC18 and an increase in tumor growth and metastasis of human melanoma cells (Jean et al, 1998). AP-2alpha was reduced in advanced Dukes's stage adenocarcinomas. Together with reduced AP-2gamma expression in high grade colorectal carcinomas, this might contribute to tumor progression (Ropponen et al, 2001). The developmentally regulated transcription factor AP-2 is expressed at higher levels in human fetal skeletal muscle and rhabdomyosarcoma cells compared to human adult skeletal muscle (Zhang et al, 1998). Through its distinct actions in adipocytes and macrophages, AP2 links features of the metabolic syndrome including insulin resistance, obesity, inflammation, and atherosclerosis (Linton and Fazio, 2003). Chromatin immunoprecipitation analysis demonstrated DNA binding activity of AP-2 in the TbetaRI promoter and of Sp1 in the TbetaRII promoter after treatment with 5-aza-2′-deoxycytidine (Zhang et al, 2005). Site-specific methylation in NF1 gene, involving transcription factor binding sites for SP1, CRE (−10), and AP-2, was observed. One region of the 5′-UTR (untranslated region) overlapping with a putative AP-2 binding site was methylated at 30-100% in 4/20 control samples (Harder et al, 2004). High resolution mapping of methylated cytosines revealed that differential expression of the AP-2 alpha gene in normal human lung fibroblasts and their SV40-transformed counterparts was associated with distinct patterns of cytosine methylation in the AP-2 alpha promoter just 5′ to the transcription initiation site. Site-specific methylation was positively correlated with increased AP-2 alpha gene expression in both transformed cell lines investigated (Zhu et al, 2001). High resolution mapping of methylated cytosines revealed that differential expression of the AP-2 alpha gene in normal human lung fibroblasts and their SV40-transformed counterparts was associated with distinct patterns of cytosine methylation in the AP-2 alpha promoter just 5′ to the transcription initiation site. Site-specific methylation was positively correlated with increased AP-2 alpha gene expression in both transformed cell lines investigated (Zhu et al, 2001).

Cdc42 effector protein 1. Cdc42 effector protein 1 (CDC42EP1 alias MSE55, CEP1, Borg5, MGC15316) is a member of the Rho GTPase family that regulates multiple cellular activities, including actin polymerization. The protein encoded by this gene is a CDC42 binding protein that mediates actin cytoskeleton reorganization at the plasma membrane. The encoded protein, which is secreted, is primarily found in bone marrow. Two transcript variants encoding different isoforms have been found for this gene (Entrez database). Northern blot analysis demonstrates expression limited to endothelial and bone marrow stromal cells, but not poly(A) RNA from monkey liver, spleen, brain, lung, and kidney. On this basis, this protein was designated MSE55, for marrow/stromal/endothelial cell protein with a molecular mass of 55,000 daltons. Its tissue-specific expression may suggest a functional role in hematopoiesis (Bahou et al, 1992). MSE55 induced the formation of long, actin-based protrusions in NIH 3T3 cells as detected by immunofluorescence and live-cell video microscopy. MSE55-induced protrusion formation was blocked by expression of dominant-negative N17Cdc42, but not by expression of dominant-negative N17Rac. These findings indicate that MSE55 is a Cdc42 effector protein that mediates actin cytoskeleton reorganization at the plasma membrane (Burbelo et al, 1999).

Glutathione peroxidase 5. Glutathione peroxidase 5 (GPX5) also known as glutathione reductase is part of the hydrogen peroxide scavenging system found within the epididymis in the mammalian male reproductive tract. GPX5 expression is epididymis-specific and the transcript is unique from other GPXs because it contains a deletion resulting in an mRNA that does not contain a selenocysteine (UGA) codon (an unusual amino acid present in other GPXs). This deletion also renders the mRNA incapable of encoding an active GPX isoenzyme. For this reason, GPX5 is selenium-independent and has very little activity towards hydrogen peroxide or organic hydroperoxides. GPX5, which is bound to the acrosome of sperm, may act to protect sperm from premature acrosome reaction in the epididymis (Entrez database). The cDNA of human GPX5 is cloned. Thereby it was shown that the majority of transcripts contain a 118 nt frame-shifting deletion, arising, most likely, from inappropriate excision of exon 3 during processing. Antisera raised against recombinant human GPX5 cross-reacted with rat and macaque (Macaca fascicularis) epididymal proteins of the size expected for full-length, active GPX5. However, no similar reactivity could be demonstrated in any of the human samples tested (Hall et al, 1998). The tissue-restricted polyoma enhancer activator protein (PEA3) of the ETS oncogene family of DNA-binding proteins is a putative modulator of the epididymis-specific glutathione peroxidase 5 gene GPX5 (Drevet et al, 1998). At least part of the androgenic control of the GPX5 expression is exerted at the transcriptional level via an androgen response element localized in the distal promoter region of the GPX5 gene (Lareyre et al, 1997).

Gamma-parvin. Gamma-parvin (PARVG) is a member of the parvin family, a family of actin-binding proteins associated with focal contacts (OMIM database).

NKG2D ligand 4 precursor. NKG2D ligand 4 precursor (RAET1E alias NKG2D ligand 4, NKG2DL4, N2DL-4, RL-4, LETAL, bA350J20.7, ULBP4; MGC125308; MGC125309; bA350J20.7) also known as Retinoic acid early transcript 1E, Lymphocyte effector toxicity activation ligand, RAE-1-like transcript 4 is a member of the RAET1 family. The members of this family are major histocompatibility complex (MHC) class I-related genes located within a 180-kb cluster on chromosome 6q24.2-q25.3. RAET1 proteins contain MHC class I-like alpha-1 and alpha-2 domains. RAET1E and RAET1G (MIM 609244) differ from the other RAET1 proteins (e.g., RAET1I, or ULBP1; MIM 605697) in that they have type I membrane-spanning sequences at their C termini rather than glycosylphosphatidylinositol anchor sequences (Radosavljevic et al., 2002). The expression of diverse NKG2D-binding molecules (RAET1E and RAET1G) in different tissues and with different properties is consistent with multiple modes of infection- or stress-induced activation (Bacon et al, 2004). Tissue expression of ULBP4 differs from other members of the family, in that it is expressed predominantly in the skin (Jan Chalupny et al, 2003).

Oncostatin M precursor. Oncostatin M precursor (OSM alias MGC20461) is a member of a cytokine family that includes leukemia-inhibitory factor, granulocyte colony-stimulating factor, and interleukin 6. This gene encodes a growth regulator which inhibits the proliferation of a number of tumor cell lines. It regulates cytokine production, including IL-6, G-CSF and GM-CSF from endothelial cells. The related members of the interleukin 6 (IL-6) family of cytokines, IL-6, leukemia inhibitory factor (LIF), and oncostatin M, act as major inflammatory mediators and induce the hepatic acute phase reaction. Normal parenchymal liver cells express the receptors for these cytokines, and these receptors activate, to a comparable level, the intracellular signaling through signal transducer and activator of transcription (STAT) proteins and extracellular-regulated kinase (ERK) (Entrez database). OSM stimulates the expression of the immediate early genes c-fos and Egr-1 in Gnv-4 cells, an effect dependent upon the activation of the MAPK Erk1/2 intracellular signaling pathway (Igaz et al, 2005). OSM and macrophage-derived cytokines suppressed proliferation of normal epithelial cells, but reduced inhibition or even stimulated proliferation was noted for preneoplastic cells (Loewen et al, 2005). In human liver, OSM protein is expressed in Kupffer cells, variably in normals but universally in cirrhosis. The differential expression pattern of OSM and its receptors could allow for differential OSM signaling by alternative utilization of receptors to promote hepatocyte proliferation in acute injury and, with its homologue LIF, for the bile ductular reaction in cirrhosis (Znoyko et al, 2005). OSM strongly and specifically affects the expression of many genes, in particular those involved with innate immunity, angiogenesis, adhesion, motility, tissue remodeling, cell cycle and transcription (Finelt et al, 2005). OSM has a strong lipid-lowering effect under in vivo conditions in which the levels of circulating LDL-C are high and liver LDLR transcription is repressed (King et al, 2005). Oncostatin M (OSM), a member of the interleukin-6 family of cytokines, is thought to be expressed mostly by activated T-lymphocytes and monocytes in adult animals. However, specific constitutive tissue expression is reported of OSM in the pancreas, kidney, testes, spleen, stomach, and brain, but not liver or lung, of three adult rodent species (Znoyko et al, 2005). These studies identify S100A9 as a novel OSM-regulated gene through the STAT3-signaling cascade and suggest its involvement in the growth regulation of breast cancer cells (Li et al, 2004). Oncostatin M stimulates the detachment of a reservoir of invasive mammary carcinoma cells (Holzer et al, 2004). Blood neutrophils can be stimulated to express and rapidly release large quantities of OSM. It is proposed that OSM is released from neutrophils as they infiltrate rheumatoid joints and, thus, contribute to the complex cytokine network that characterizes retinoic acid (Cross et al, 2004). Oncostatin-M may contribute to the process of healing after myocardial infarction (Gwechenberger et al, 2004). Oncostatin M expression is upregulated in cirrhosis where it may have a role as a profibrogenic cytokine in hepatic stellate cells (Levy et al, 2000). OSM induces an angiogenic effect on capillary endothelial cells, which could be, at least in part, implicated in pathological processes such as atherosclerosis or tumor growth (Vasse et al, 1999).

Cytohesin-4. Cytohesin-4 (PSCD4 alias CYT4) also known as Pleckstrin homology, Sec7 and coiled/coil domains 4 (PSCD4) is a member of the PSCD family. Members of this family have identical structural organization that consists of an N-terminal coiled-coil motif, a central Sec7 domain, and a C-terminal pleckstrin homology (PH) domain. The coiled-coil motif is involved in homodimerization, the Sec7 domain contains guanine-nucleotide exchange protein (GEP) activity, and the PH domain interacts with phospholipids and is responsible for association of PSCDs with membranes. Members of this family appear to mediate the regulation of protein sorting and membrane trafficking. The PSCD4 exhibits GEP activity in vitro with both ARF1 and ARF5 but is inactive with ARF6. The PSCD4 and PSCD1 gene structures are very similar (Entrez database).

Solute carrier family 22 (organic cation transporter) member 1. Polyspecific organic cation transporters in the liver, kidney, intestine, and other organs are critical for elimination of many endogenous small organic cations as well as a wide array of drugs and environmental toxins. Solute carrier family 22 (organic cation transporter) member 1 (SLC22A1 alias OCT1) is one of three similar cation transporter genes located in a cluster on chromosome 6. The encoded protein contains twelve putative transmembrane domains and is a plasma integral membrane protein. Two transcript variants encoding two different isoforms have been found for this gene, but only the longer variant encodes a functional transporter (Entrez database). OCT1 is the main organic cation uptake system in hepatocytes and has common features with organic cation uptake over the basolateral membrane of renal proximal tubules (Grundemann et al., 1994). Together with other 24 membrane transporters OCT1 have potential roles in drug response, as determined by phylogenetic and population-genetics analysis in a large population (Leabman et al., 2003). Changes at evolutionarily conserved positions of OCT1 are strong predictors of decreased function and suggested that a combination of evolutionary conservation and chemical change might be a stronger predictor of function (Shu et al., 2003). hOCT1, which is expressed in the intestine and liver, is likely to play a major role in the intestinal absorption and hepatic disposition of ranitidine and famotidine in humans (Bourdet et al., 2005). OCT1 and OCT2 mediate luminal Ach (acetylcholine) release in human airways and suggest that ACh release is blocked after inhalation of budesonide (Lips et al., 2005). The expression of organic cation transporters rOCT1 and rOCT2 is reduced in experimental diabetes in rats (Grover et al., 2004). When mice were given metformin, the blood lactate concentration significantly increased in the wild-type mice, whereas only a slight increase was observed in Oct1(−/−) mice. The plasma concentration of metformin exhibited similar time profiles between the wild-type and Oct1(−/−) mice, suggesting that the liver is the key organ responsible for the lactic acidosis and the Oct1 gene is involved in lactic acidosis caused by metformin (Wang et al., 2003).

Tyrosine-protein kinase-like 7 precursor. Tyrosine-protein kinase-like 7 precursor (PTK7 alias CCK4 (Colon carcinoma kinase 4, CCK-4) is a receptor protein tyrosine kinase which transduce extracellular signals across the cell membrane. A subgroup of these kinases lack detectable catalytic tyrosine kinase activity but retain roles in signal transduction. The protein encoded by this gene is a member of this subgroup of tyrosine kinases and may function as a cell adhesion molecule. This gene is thought to be expressed in colon carcinomas but not in normal colon, and therefore may be a marker for or may be involved in tumor progression. Five transcript variants encoding five different isoforms have been found for this gene (Entrez database).

Cytidine monophosphate-N-acetylneuraminic acid hydroxylase. Cytidine monophosphate-N-acetylneuraminic acid hydroxylase (CMAH) is also known as CMP-N-acetylneuraminate monooxygenase, CMP-NeuAc hydroxylase, CMP-Neu5Ac hydroxylase, CMP-sialic acid hydroxylase (CSAH), CMP-N-acetylneuraminic acid hydroxylase). Sialic acids are terminal components of the carbohydrate chains of glycoconjugates involved in ligand-receptor, cell-cell, and cell-pathogen interactions. The two most common forms of sialic acid found in mammalian cells are N-acetylneuraminic acid (Neu5Ac) and its hydroxylated derivative, N-glycolylneuraminic acid (Neu5Gc). Studies of sialic acid distribution show that Neu5Gc is not detectable in normal human tissues although it was an abundant sialic acid in other mammals. Neu5Gc is, in actuality, immunogenic in humans. The absense of Neu5Gc in humans is due to a deletion within the human gene CMAH encoding cytidine monophosphate-N-acetylneuraminic acid hydroxylase, an enzyme responsible for Neu5Gc biosynthesis. Sequences encoding the mouse, pig, and chimpanzee hydroxylase enzymes were obtained by cDNA cloning and found to be highly homologous. However, the homologous human cDNA differs from these cDNAs by a 92-bp deletion in the 5′ region. This deletion, corresponding to exon 6 of the mouse hydroxylase gene, causes a frameshift mutation and premature termination of the polypeptide chain in human. It seems unlikely that the truncated human hydroxylase mRNA encodes for an active enzyme explaining why Neu5Gc is undetectable in normal human tissues. Human genomic DNA also shows evidence of this deletion which does not occur in the genomes of African great apes. Nonetheless, the CMAH gene maps to 6p21.32 in humans and great apes indicating that mutation of the CMAH gene occurred following human divergence from chimpanzees and bonobos (Entrez database). Studies indicate that the CMAH gene is inactivated shortly before the time when brain expansion began in humankind's ancestry, approximately 2.1-2.2 mya. In this regard, it is of interest that although Neu5Gc is the major sialic acid in most organs of the chimpanzee, its expression is selectively down-regulated in the brain (Chou et al, 2002).

Solute carrier family 24 (sodium/potassium/calcium exchanger), member 3. Solute carrier family 24 (sodium/potassium/calcium exchanger) member 3 is also known as SLC24A3 or NCKX3. The majority of TM neurones express NCX1, NCX2 and NCKX3 (Sergeeva et al, 2003). The N-terminal region of NCKX3, although not essential for expression, increased functional activity at least 10-fold and may represent a cleavable signal sequence. NCKX3 transcripts were most abundant in brain, with highest levels found in selected thalamic nuclei, in hippocampal CA1 neurons, and in layer IV of the cerebral cortex. Many other tissues also expressed NCKX3 at lower levels, especially aorta, uterus, and intestine, which are rich in smooth muscle (Kraev et al, 2001).

Somatostatin receptor type 3. Somatostatin receptor type 3 is also known as SS3R, SSR-28 or SSTR3. Somatostatin acts at many sites to inhibit the release of many hormones and other secretory proteins. The biological effects of somatostatin are probably mediated by a family of G protein-coupled receptors that are expressed in a tissue-specific manner. SSTR3 is a member of the superfamily of receptors having seven transmembrane segments and is expressed in highest levels in brain and pancreatic islets. SSTR3 is functionally coupled to adenylyl cyclase (Entrez database). Five SSTR subtypes are variably expressed at the mRNA level in breast tumors with 91% of samples showing SSTR1, 98% SSTR2, 96% SSTR3, 76% SSTR4, and 54% SSTR5. Levels of SSTR mRNA, when corrected for beta-actin levels, were highest for SSTR3 (Kumar et al, 2005). All five SSTRs were differentially expressed as membrane and cytoplasmic proteins in cortical neurons with significant variations in control vs. alzheimer diseased (AD) brain. In AD cortical brain region, somatostatin and neuropeptide-Y-positive neurons decreased (>70%). SSTR3 was the only receptor subtype that increased in AD cortex (Kumar, 2005). SSTR3 is expressed in the HCC cells, but not in the L-02 cells, which suggests a molecular basis for the HCC-selective effects of octreotide (Liu et al, 2004). SSTR3 protein existed in the membrane of gastric cancer cells. In normal gastric mucosa, SSTR3 protein distributed to the cellular membrane and cytoplasm or interstitial tissue in submucosa. The expression of SSTR3 protein was significantly lower in gastric cancer compared with normal mucosa. Moreover, the poor-differentiated adenocarcinoma was lower than the well-differentiated adenocarcinoma, and the similar result in cell lines. Octreotide could inhibit the growth and induce the apoptosis of gastric cancer and normal epithelial cells that expressed SSTR3, but didn't affect the cells with no or weakly expression of SSTR3 (Hu et al, 2004). SSTR3 mRNA is confined to the pituitary, hypothalamus, and spinal cord from early to midgestation (Goodyer et al, 2004). In vitro, octreotide inhibited the proliferation, invasion, and differentiation of HUVECs elicited by VEGF. RT-PCR analysis demonstrated that HUVECs expressed the somatostatin receptor subtype SSTR3. In vivo, octreotide was sufficiently potent to suppress nude mice corneal neovascularization induced by tumor tissues from LCI-D20 (Jia et al, 2003). In renal cell tumors, SSTR3 transcripts were completely absent. In breast cancer tissue, SSTR subtypes were transcribed independently of patient age, menstrual status, diagnosis, histological grade, and levels of estrogen receptor and progesterone receptor (Vikic-Topic et al, 1995).

Bone morphogenetic protein 7 precursor. Bone morphogenetic protein 7 precursor (BMP-7) is also known as Osteogenic protein 1 (OP-1) or Eptotermin alfa. The bone morphogenetic proteins (BMPs) are a family of secreted signaling molecules that can induce ectopic bone growth. Many BMPs are part of the transforming growth factor-beta (TGFB) superfamily. BMPs were originally identified by an ability of demineralized bone extract to induce endochondral osteogenesis in vivo in an extraskeletal site. Based on its expression early in embryogenesis, the BMP encoded by this gene has a proposed role in early development. In addition, the fact that this BMP is closely related to BMP5 and BMP7 has lead to speculation of possible bone inductive activity (Entrez database).

Caspase recruitment domain protein 10. Caspase recruitment domain protein 10 (CARD10) is also known as CARD-containing MAGUK protein 3 (CARMA 3). The caspase recruitment domain (CARD) is a protein module that consists of 6 or 7 antiparallel alpha helices. It participates in apoptosis signaling through highly specific protein-protein homophilic interactions. CARDs induce nuclear factor kappa-B (NFKB; MIM 164011) activity through the IKK (e.g., IKBKB; MIM 603258) complex. CARD9 (MIM 607212), CARD10, CARD11 (MIM 607210), and CARD14 (MIM 607211) interact with BCL10 (MIM 603517) and are involved in NFKB signaling complexes. Except for CARD9, these CARD proteins are members of the membrane-associated guanylate kinase (MAGUK) family (OMIM database). CARMA3 physically associate with Ikappa kinase gamma/NFkappaB essential modulator (IkappaKgamma-NEMO) in lymphoid and non-lymphoid cells. Expression of the NEMO-binding region of CARMA3 exerts a dominant negative effect on BCL10-mediated activation of NfkappaB (Stilo et al, 2004). CARD10 is a novel BCL10 interactor that belongs to the membrane-associated guanylate kinase family, a class of proteins that function to organize signaling complexes at plasma membranes. When expressed in cells, CARD10 binds to BCL10 and signals the activation of NF-kappaB through its N-terminal effector CARD domain. It is proposed that CARD10 functions as a molecular scaffold for the assembly of a BCL10 signaling complex that activates NF-kappaB (Wang et al, 2001).

Neutrophil cytosol factor 4. Neutrophil cytosol factor 4 (NCF-4), also known as Neutrophil NADPH oxidase factor 4 or p40-phox, is a cytosolic regulatory component of the superoxide-producing phagocyte NADPH-oxidase, a multicomponent enzyme system important for host defense. This protein is preferentially expressed in cells of myeloid lineage. It interacts primarily with neutrophil cytosolic factor 2 (NCF2/p67-phox) to form a complex with neutrophil cytosolic factor 1 (NCF1/p47-phox), which further interacts with the small G protein RAC1 and translocates to the membrane upon cell stimulation. This complex then activates flavocytochrome b, the membrane-integrated catalytic core of the enzyme system. The PX domain of this protein can bind phospholipid products of the PI(3) kinase, which suggests its role in PI(3) kinase-mediated signaling events. The phosphorylation of this protein was found to negatively regulate the enzyme activity. Alternatively spliced transcript variants encoding distinct isoforms have been observed (Entrez database).

Cadherin EGF LAG seven-pass G-type receptor 1 precursor. Cadherin EGF LAG seven-pass G-type receptor 1 precursor (CELSR1, ME2, CDHF9) also known as Flamingo homolog 2 (HFM12, FM12). is a member of the flamingo subfamily, part of the cadherin superfamily. The flamingo subfamily consists of nonclassic-type cadherins; a subpopulation that does not interact with catenins. The flamingo cadherins are located at the plasma membrane and have nine cadherin domains, seven epidermal growth factor-like repeats and two laminin A G-type repeats in their ectodomain. They also have seven transmembrane domains, a characteristic unique to this subfamily. It is postulated that these proteins are receptors involved in contact-mediated communication, with cadherin domains acting as homophilic binding regions and the EGF-like domains involved in cell adhesion and receptor-ligand interactions. This particular member is a developmentally regulated, neural-specific gene which plays an unspecified role in early embryogenesis (Entrez database). Expression of the CELSR/Flamingo homologue, c-fmi1, in the early avian embryo indicates a conserved role in neural tube closure and additional roles in asymmetry and somitogenesis (Formstone and Mason, 2005). Each CELSR is expressed prominently in the developing brain following a specific pattern, suggesting that they serve distinct functions (Tissir et al, 2002).

Platelet-derived growth factor B chain precursor. Platelet-derived growth factor B chain precursor (PDGF B-chain, PDGFB, SIS, SSV, PDGF2, c-sis) is a member of the platelet-derived growth factor family. The four members of this family are mitogenic factors for cells of mesenchymal origin and are characterized by a motif of eight cysteines. This gene product can exist either as a homodimer (PDGF-BB) or as a heterodimer with the platelet-derived growth factor alpha polypeptide (PDGF-AB), where the dimers are connected by disulfide bonds. Mutations in this gene are associated with meningioma. Reciprocal translocations between chromosomes 22 and 7, at sites where this gene and that for COL1A1 are located, are associated with a particular type of skin tumor called dermatofibrosarcoma protuberans resulting from unregulated expression of growth factor. Two splice variants have been identified for this gene (Entrez database). Medulloblastomas contained the highest amounts of PDGF B-chain, some four to eight times more than that in control brain tissue. Tumors that contained a high level of PDGF B-chain showed high proliferative activity (Nakamura et al, 1993). Trichostatin A (TSA) activates reporter gene constructs driven by the human platelet-derived growth factor B (PDGF-B) gene promoter. This activation showed an inverse correlation with the cell type-specific transcriptional activities of the promoter (Ulleras et al, 2001). Tissue specificity was not clear in the 5′ upstream region alone, and regulation by gene methylation or by elements other than in the 5′ region seemed to be necessary (Takimoto et al, 1993). Platelet-derived growth factor B-chain was also abnormally methylated in 4 of 13 (31%) multinodular goiters (MNG), 17 of 24 (71%) follicular adenomas (FA), and 9 of 13 (69%) papillary carcinomas (PC) (Matsuo et al, 1993). Gene expression of PDGF-A and PDGF-B mRNA were increased 22- and 6-fold, respectively, in biopsies from patients with diabetic nephropathy compared with control tissue (Langham et al, 2003). Adult SPC-PDGFB transgenic mice exhibited lung pathology characterized by enlarged airspaces, inflammation, and fibrosis (Hoyle et al, 1999). The transition between hyperplasia (complete hydatidiform mole) and neoplasia (choriocarcinoma) in these cells correlates with at least a 10- to 20-fold activation of the PDGF-B gene (Holmgren et al, 1993).

Transmembrane protease serine 6. Transmembrane protease serine 6 (TMPRSS6) is a type II transmembrane serine proteinase that is found attached to the cell surface. The encoded protein may be involved in matrix remodeling processes in the liver (Entrez database).

Bcl-2 interacting killer. Bcl-2 interacting killer (BIK, BP4, BIP1, BBC1) also known as Apoptosis inducer NBK (NBK) is known to interact with cellular and viral survival-promoting proteins, such as BCL2 and the Epstein-Barr virus in order to enhance programmed cell death. Because its activity is suppressed in the presence of survival-promoting proteins, this protein is suggested as a likely target for antiapoptotic proteins. This protein shares a critical BH3 domain with other death-promoting proteins, BAX and BAK (Entrez database). Human blk is expressed only in B lymphocytes (Drebin et al, 1995). The two human blk RNAs arise from the transcription of the blk gene by two distinct promoters and that these promoters may be subject to regulation by different trans-acting factors (Lin et al, 1995). P55blk and p53/p56lyn may be particularly good candidates for the membrane immunoglobulin-activated tyrosine kinase (Law et al, 1992). There is a role of blk kinase in anti-mu-mediated pathway to cell cycle arrest (Yao et al, 1993). Blk is down-regulated in a clinically distinct aggressive subset of B-CLL completely resistant in vitro to irradiation-induced apoptosis (Vallat et al, 2003). Despite the absence of Blk, the development, in vitro activation, and humoral immune responses of B cells to T-cell-dependent and -independent antigens are unaltered. These data are consistent with functional redundancy of Blk in B-cell development and immune responses (Texido et al, 2000). Expression of constitutively active Blk in the T lineage resulted in the appearance of clonal, thymic lymphomas composed of intermediate single positive cells (Malek et al, 1998).

PKHD1. PKHD1 stands for polycystic kidney and hepatic disease 1 (autosomal recessive) and is also known as FCYT, ARPKD, TIGM1. The protein encoded by this gene is predicted to have a single transmembrane (TM)-spanning domain and multiple copies of an immunoglobulin-like plexin-transcription-factor domain. Alternative splicing results in two transcript variants encoding different isoforms. Other alternatively spliced transcripts have been described, but the full length sequences have not been determined. Several of these transcripts are predicted to encode truncated products which lack the TM and may be secreted. Mutations in this gene cause autosomal recessive polycystic kidney disease, also known as polycystic kidney and hepatic disease-1 (Entrez database). HNF-1beta mutant mice show decreased expression of Pkhd1, the gene that is mutated in humans with autosomal-recessive polycystic kidney disease (ARPKD) (Igarashi et al, 2005). A total of 263 different PKHD1 mutations (639 mutated alleles) are included in the locus-specific database. Except for a few population-specific founder alleles and the common c.107C>T (p.Thr36Met) missense change, PKHD1 is characterized by significant allelic diversity (Bergmann et al, 2005). Polyductin is part of the group of polycystic kidney disease (PKD)-related proteins expressed in primary apical cilia. It probably serves in other subcellular functional roles. The detection of three different products using two antisera, with evidence for distinct subcellular localizations, suggests that PKHD1 encodes membrane-bound and soluble isoforms (Menezes et al, 2004). Renal cyst formation is accompanied by a drastic defect in the transcriptional activation of Umod, Pkhd1 and Pkd2 genes, whose mutations are responsible for distinct cystic kidney syndromes. In vivo chromatin immunoprecipitation experiments demonstrated that HNF1beta binds to several DNA elements in murine Umod, Pkhd1, Pkd2 and Tg737/Polaris genomic sequences (Gresh et al 2004). During embryogenesis, PKHD1 is widely expressed in epithelial derivatives, including neural tubules, gut, pulmonary bronchi, and hepatic cells. In the kidneys of the pck rats, the rat model of which is genetically homologous to human ARPKD, the level of PKHD1 was significantly reduced but not completely absent. In cultured renal cells, the PKHD1 gene product colocalized with polycystin-2, the gene product of autosomal dominant polycystic disease type 2, at the basal bodies of primary cilia (Zhang et al, 2004). PKHD1 was identified to be mutated in ARPKD (Onuchic et al, 2002).

Myosin-18B. Myosin-18B, also known as Myosin XVIIIb alias BK125H2.1, may regulate muscle-specific genes when in the nucleus and may influence intracellular trafficking when in the cytoplasm. The encoded protein functions as a homodimer and may interact with F actin. Mutations in this gene are associated with lung cancer (Entrez database). Genetic and epigenetic alterations of the MYO18B gene was analyzed in colorectal cancers. Alleic imbalance at the MYO18B locus was detected in 16 of 43 (40%) informative cases. Mutations of the MYO18B gene were detected in 2 of 11 (18%) cell lines and 1 of 47 (2%) surgical specimens. Nine of 11 (82%) cell lines showed reduced MYO18B expression, which was restored in all 9 by treatment with 5-aza-2′-deoxycytidine and/or trichostatin A (TSA). Although hypermethylation of the promoter CpG island for MYO18B was not detected, a significant correlation was observed between the level of MYO18B expression and the level of acetylation of histones H3 and H4 in 6 cell lines with and without TSA treatment (Nakano et al, 2005). Missense MYO18B mutations were detected in 1 of 4 (25%) ovarian cancer cell lines and in 1 of 17 (5.9%) primary ovarian cancers. MYO18B expression was reduced in all 4 ovarian cancer cell lines and in 12 of 17 (71%) of primary ovarian cancers. MYO18B expression was restored by treatment with 5-aza-2′-deoxycytidine and/or trichostatin A in 3 of 4 cell lines with reduced MYO18B expression, and hypermethylation of the promoter CpG island for MYO18B was observed in 2 of these 3 cell lines. Its hypermethylation was also observed in 2 of 15 (13%) primary ovarian cancers (Yanaihara et al, 2004). MYO18B, located at chromosome 22q12.1 and found that it is frequently deleted, mutated, and hypermethylated in lung cancers (Nishioka et al, 2002).

GAS2-likeprotein 1. GAS2-like protein 1 (GAS2L1) is also known as Growth arrest-specific 2-like 1 or GAS2-related protein on chromosome 22 (GAR22 protein, GAR22). The protein encoded by this gene, a member of the GAS2 family, is similar in sequence to the mouse protein Gas2, an actin-associated protein expressed at high levels in growth-arrested cells. Expression of the mouse Gas2 gene is negatively regulated by serum and growth factors. Three transcript variants encoding two different isoforms have been found for this gene (Entrez database). Although hGAR22 and mGAR22 mRNAs are expressed nearly ubiquitously, mGAR22 protein can only be detected in testis and brain.

Furthermore, only the beta isoform is present in these tissues. GAR22beta expression is induced in a variety of cultured cells by growth arrest. The absolute amounts of GAR22beta protein expressed are low (Goriounov et al, 2003). The regulation of Gas2 biosynthesis reflects the pattern of mRNA expression: its relative level is tightly associated with growth arrest. Gas2 seems to be regulated also at the posttranslational level via a phosphorylation mechanism (Brancolini et al, 1992).

Ras and Rab interactor 2. Ras and Rab interactor 2 (RIN2, RASSF4) The RAB5 protein is a small GTPase involved in membrane trafficking in the early endocytic pathway. The protein encoded by this gene binds the GTP-bound form of the RAB5 protein preferentially over the GDP-bound form, and functions as a guanine nucleotide exchange factor for RAB5. The encoded protein is found primarily as a tetramer in the cytoplasm and does not bind other members of the RAB family (Entrez database). RASSF4 (AD037) shows approximately 25% identity with RASSF1A and 60% identity with RASSF2. RASSF4 binds directly to activated K-Ras in a GTP-dependent manner via the effector domain, thus exhibiting the basic properties of a Ras effector. Overexpression of RASSF4 induces Ras-dependent apoptosis in 293-T cells and inhibits the growth of human tumor cell lines. Although broadly expressed in normal tissue, RASSF4 is frequently down-regulated by promoter methylation in human tumor cells. Thus, RASSF4 appears to be a new member of the RASSF family of potential Ras effector/tumor suppressors (Eckfeld et al, 2004). It was demonstrated that the expression of RASSF4/AD037 was lost in 12.5% (1/8) of NPC cell lines/xenografts. Bisulfite sequencing analysis revealed dense methylation in the promoter region of RASSF4/AD037 in the cell line. Restoration of RASSF4/AD037 mRNA was observed by treatment with a demethylating agent (Chow et al, 2004).

Forkhead box C1. Forkhead box C1 (FOXC1, FKHL7, IRID1, FREAC3, ARA, IGDA, IHG1) belongs to the forkhead family of transcription factors which is characterized by a distinct DNA-binding forkhead domain. The specific function of this gene has not yet been determined; however, it has been shown to play a role in the regulation of embryonic and ocular development. Mutations in this gene cause various glaucoma phenotypes including primary congenital glaucoma, autosomal dominant iridogoniodysgenesis anomaly, and Axenfeld-Rieger anomaly (Entrez database).

GRB2-related adaptor protein 2. GRB2-related adaptor protein 2 (GRAP2, Grf40, GrbX, GRBLG, GADS, Mona, P38; GRID; GRPL; GRB2L; GRAP-2) a member of the GRB2/Sem5/Drk family. This member is an adaptor-like protein involved in leukocyte-specific protein-tyrosine kinase signaling. Like its related family member, GRB2-related adaptor protein (GRAP), this protein contains an SH2 domain flanked by two SH3 domains. This protein interacts with other proteins, such as GRB2-associated binding protein 1 (GAB1) and the SLP-76 leukocyte protein (LCP2), through its SH3 domains. Transcript variants utilizing alternative polyA sites exist (Entrez database). GRAP2 and GPR51, were found to respond to low-dose radiation but not to high-dose radiation in G1-arrested normal human skin fibroblasts (Ding et al, 2005). Gads adaptor protein is expressed in many hematopoietic tissues, including bone marrow, lymph node, and spleen. Using intracellular staining, Gads protein was detected in a number of cells, including B cells, T cells, NK cells, monocytes, and plasmacytoid DC, but not in macrophages, neutrophils, or monocyte-derived DC. Gads may have a negative regulatory role in signaling through survival pathways, and is necessary for normal homeostatic proliferation in B cells (Yankee et al, 2005). GRAP genes were up-regulated in salivary glands of the MRL/lpr (an Sjogren's syndrome (SS) mouse model) (Shiraiwa et al, 2004). −2000 to +150 genomic region relative to the Mona gene transcription start site is sufficient to direct specific reporter gene expression in T cell lines, Jurkat, and MOLT-4 and in the immature myeloid cell lines, KG1a and RC2A. Deletion analysis and electrophoresis mobility shift assay identified several cis regulatory elements: overlapping initiator sequences, one interferon response factor-2 (IRF-2)-binding site at position −154, one GC box recognized by Sp1 and Sp3 at position −52, and two acute myeloid leukemia (AML)-1 binding sites at positions −70 and −13 (Guyot and Mouchiroud, 2003). It was reported that a lineage-restricted transcription of the Mona gene is controlled by specific promoters (Guyot et al, 2002).

RASSF2. RASSF2 stands for Ras association (RalGDS/AF-6) domain family 2 and is also known as KIAA0168 or DKFZp781O1747. This gene encodes a protein that contains a Ras association domain. Similar to its cattle and sheep counterparts, this gene is located near the prion gene. The specific function of this gene has not yet been determined. Three alternatively spliced transcript variants of this gene encoding two distinct isoforms have been reported (Entrez database). Hypermethylation of RASSF2 in at least one of the regions examined was detected in seven (70%) of the 10 gastric cancer cell lines; two (20%) exhibited hypermethylation in all the regions examined including the transcription start site and lost expression of RASSF2 mRNA, which could, however, be restored by 5-aza-2′ deoxycytidine treatment, while the other five (50%) cell lines exhibited hypermethylation at the 5′- and/or 3′-edge, with four of them expressing RASSF2 mRNA. In primary gastric cancers and corresponding non-neoplastic gastric epithelia, frequencies of RASSF2 methylation ranged from 29% (23 out of 78) to 79% (62 out of 78) and 3% (two out of 78) to 60% (47 out of 78), respectively, at different CpG sites examined (Endoh et al, 2005). Aberrant methylation and histone deacetylation of RASSF2 was associated with the gene's silencing in CRC. The activities of RASSF2, which were distinct from those of RASSF1, included induction of morphologic changes and apoptosis; moreover, its ability to prevent cell transformation suggests that RASSF2 acts as a tumor suppressor in CRC. Primary CRCs that showed K-ras/BRAF mutations also frequently showed RASSF2 methylation, and inactivation of RASSF2 enhanced K-ras-induced oncogenic transformation. RASSF2 methylation was also frequently identified in colorectal adenomas (Akino et al, 2005). RASSF2A is frequently inactivated in CRCs by CpG island promoter hypermethylation, and that epigenetic (RASSF2A) and genetic (K-ras) changes are mutually exclusive and provide alternative pathways for affecting Ras signalling (Hesson et al, 2005). RASSF2 binds directly to K-Ras in a GTP-dependent manner via the Ras effector domain. However, RASSF2 only weakly interacts with H-Ras. Moreover, RASSF2 promotes apoptosis and cell cycle arrest and is frequently down-regulated in lung tumor cell lines (Vos et al, 2003).

Glutamate receptor, ionotropic kainate 2 precursor. Glutamate receptor, ionotropic kainate 2 precursor (GRIK2 RP3-438O4.2 (Vega gene ID)) is also known as Glutamate receptor 6 (GluR-6) or Excitatory amino acid receptor 4 (EAA4). This gene encodes a subunit of a kainate glutamate receptor. Glutamate receptors mediate the majority of excitatory neurotransmission in the brain. This receptor may have a role in synaptic plasticity and may be important for learning and memory. It also may be involved in the transmission of light information from the retina to the hypothalamus. The structure and function of the encoded protein is changed by RNA editing. Alternatively spliced transcript variants encoding distinct isoforms have been described for this gene (Entrez database). Histone methylation marks were studied at proximal promoters of 16 ionotropic and metabotropic glutamate receptor genes (GRIN1,2A-D; GRIA1,3,4; GRIK2,4,5; GRM1,3,4,6,7) in cerebellar cortex collected across a wide age range from midgestation to 90 years old. Levels of di- and trimethylated histone H3-lysine 4, which are associated with open chromatin and transcription, showed significant differences between promoters and a robust correlation with corresponding mRNA levels in immature and mature cerebellar cortex. In contrast, levels of trimethylated H3-lysine 27 and H4-lysine 20, two histone modifications defining silenced or condensed chromatin, did not correlate with transcription but were up-regulated overall in adult cerebellum (Stadler et al, 2005). Maternal transmission disequilibrium of GRIK2 was observed with a significance of p=0.03 (Bah et al, 2004). Deletion data singled out GRIK2 as the gene most frequently affected by deletions of 6q in acute lymphocytic leukemia (ALL). Sequence analysis of GRIK2 in 14 ALL cases carrying heterozygous 6q deletions revealed a constitutional and paternally inherited C to G substitution in exon 6 encoding for an amino acid change in one patient. The substitution was absent among 232 normal alleles tested, leaving open the possibility that heterozygous carriers of such mutations may be susceptible to ALL. Although low in all normal hematopoietic tissues, quantitative reverse transcription-PCR showed higher baseline GRIK2 expression in thymus and T cells than other lineages. Among T-cell ALL patients, 6q deletion was associated with a statistically significant reduction in GRIK2 expression (P=0.0001). By contrast, elevated GRIK2 expression was measured in the myelomonocytic line THP-1 and in one patient with common ALL. Finally, significant levels of GRIK2 expression were detected in prostate, kidney, trachea, and lung, raising the possibility that this gene may be protective against multiple tumor types (Sinclair et al, 2004).

T-box transcription factor TBX1. T-box transcription factor TBX1 is also known as T-box protein 1, or Testis-specific T-box protein (TBX1, DGS, TGA, CAFS, CTHM, DGCR, DORV, VCFS, TBX1C). This gene is a member of a phylogenetically conserved family of genes that share a common DNA-binding domain, the T-box. T-box genes encode transcription factors involved in the regulation of developmental processes. This gene product shares 98% amino acid sequence identity with the mouse ortholog. DiGeorge syndrome (DGS)/velocardiofacial syndrome (VCFS), a common congenital disorder characterized by neural-crest-related developmental defects, has been associated with deletions of chromosome 22q11.2, where this gene has been mapped. Studies using mouse models of DiGeorge syndrome suggest a major role for this gene in the molecular etiology of DGS/VCFS. Several alternatively spliced transcript variants encoding different isoforms have been described for this gene (Entrez database). Association was demonstrated between variants and haplotypes of remaining TBX1 gene and manifestations of congenital heart defects in 22q11.2 deletion patients (Rauch et al, 2004). Tbx1 haploinsufficiency causes aortic arch abnormalities in mice because of early growth and remodeling defects of the fourth pharyngeal arch arteries (Vitelli et al, 2002). Tbx1 may trigger signals from the pharyngeal endoderm directed to the underlying mesenchyme. Expression patterns of Fgf8 and Fgf10, which partially overlap with Tbx1 expression pattern, are altered in Tbx1 (−/−) mutants (Vitelli et al, 2002). RA could produce an altered Tbx1 expression pattern in zebrafish. In addition, RA could repress Tbx1 expression in a dose-dependant manner (Zhang et al, 2006).

Cadherin-like 22. Cadherin-like 22 (CDH22, C20orf25 dJ998H6.1 MGC39564 is a member of the cadherin superfamily. The gene product is composed of five cadherin repeat domains and a cytoplasmic tail similar to the highly conserved cytoplasmic region of classical cadherins. Expressed predominantly in the brain, this putative calcium-dependent cell adhesion protein may play an important role in morphogenesis and tissue formation in neural and non-neural cells during development and maintenance of the brain and neuroendocrine organs (Entez database).

Nuclear factor of activated T-cells cytoplasmic 2. Nuclear factor of activated T-cells cytoplasmic 2 (NFATC2) is also known as T cell transcription factor NFAT1 or NFAT pre-existing subunit (NF-ATP, NFATp). This gene is a member of the nuclear factor of activated T cells (NFAT) family. The product of this gene is a DNA-binding protein with a REL-homology region (RHR) and an NFAT-homology region (NHR). This protein is present in the cytosol and only translocates to the nucleus upon T cell receptor (TCR) stimulation, where it becomes a member of the nuclear factors of activated T cells transcription complex. This complex plays a central role in inducing gene transcription during the immune response. Alternate transcriptional splice variants, encoding different isoforms, have been characterized (Entrez database). The expression levels of the NFAT family members NFAT1, -2, and -4 were normal in the SCID patients' T cells, dephosphorylation and nuclear translocation of these NFAT proteins occurred very transiently and incompletely upon stimulation (Feske et al, 2000). Two of the seven kinase inhibitors, staurosporine (St) and bisindolylmaleimide I (BI), resulted in the dephosphorylation and nuclear localization of NFATp. Treatment of cells with ionomycin resulted in NFATp dephosphorylation and nuclear localization (Feske et al, 2000). NFAT1 mRNA is preferentially expressed in mature CD4(+) single-positive cells (Amasaki et al, 2000). Continued culture in the presence of polarizing cytokines established a selective pattern of histone acetylation on both cytokine genes. This correlated with restricted access of the transcription factor NFAT1 to these gene regulatory regions as well as mutually exclusive gene expression by the differentiated T cells (Avni et al, 2001). The level of NFATc2 binding to NFAT motifs in the CD3gamma gene promoter was greatly increased in the abnormal T cells from hypereosinophilic syndrome (Willard-Gallo et al, 2005).

MyoD family inhibitor. MyoD family inhibitor (MDFI, I-MF) is also known as Myogenic repressor I-mf. It is a transcription factor that negatively regulates other myogenic family proteins. Studies of the mouse homolog, 1-mf, show that it interfers with myogenic factor function by masking nuclear localization signals and preventing DNA binding. Knockout mouse studies show defects in the formation of vertebrae and ribs that also involve cartilage formation in these structures (Entrez database). I-mfa domain proteins interact with the Axin complex and affect Axin regulation of both the Wnt and the JNK activation pathways (Kusano S and Raab-Traub, 2002). I-mfa is expressed at a low level in an osteoblast-like cell line, MC3T3E1, and a pluripotent differentiation modulator, 1,25-dihydroxyvitamin D(3), specifically enhanced 1-mfa mRNA expression (Tsuji et al, 2001). I-mfa plays an important role in trophoblast and chondrogenic differentiation by negatively regulating a subset of lineage-restricted bHLH proteins (Kraut et al, 1998).

TGM3. TGM3 alias TGE, MGC126249 or MGC126250 stands for transglutaminase 3 (E polypeptide, protein-glutamine-gamma-glutamyltransferase). Transglutaminases are enzymes that catalyze the crosslinking of proteins by epsilon-gamma glutamyl lysine isopeptide bonds. While the primary structure of transglutaminases is not conserved, they all have the same amino acid sequence at their active sites and their activity is calcium-dependent. The protein encoded by this gene consists of two polypeptide chains activated from a single precursor protein by proteolysis. The encoded protein is involved the later stages of cell envelope formation in the epidermis and hair follicle (Entrez database). Significantly higher levels of keratin (Ker)-14 and -17 mRNAs, combined with lower levels of Ker-4, Ker-13 and transglutaminase 3 (TG-3) transcripts, were observed in OSCC (Oral Squamous Cell Carcinoma) and severely dysplastic tissues, whereas this expression profile was reversed in hyperplasia and in mild to moderate dysplasia (Ohkura et al, 2005). TGM3 plays a important role in the epidermis differentiation in embryogenesis (Zhang et al, 2005). Transglutaminase 1, 2, and 3 could be involved in cross-linking of huntingtin into intranuclear inclusions in HD (Huntington disease). It was suggested that inhibiting transglutaminase should be explored as a potential treatment strategy for HD (Zainelli et al, 2005). Immunostaining for transglutaminase3 was absent or faint throughout almost the entire suprabasal epidermis in NTS (Netherton Syndrome) (Raghunath et al, 2004). It was revealed that genes involved in squamous cell differentiation were coordinately downregulated, including annexin I, small proline-rich proteins (SPRRs), calcium-binding S100 proteins (S100A8, S100A9), transglutaminase (TGM3), cytokeratins (KRT4, KRT13), gut-enriched Krupple-like factor (GKLF) and cystatin A, in human esophageal squamous cell carcinoma (ESCC) (Luo et al, 2004). TGM3 is downregulated in head and neck squamous cell carcinoma (Gonzalez et al, 2003). Immunohistochemical analysis of the skin revealed that the enzyme is present in the cells of the granular and cornified layers consistent with its role in cornified envelope formation. In cultured keratinocytes, TGase 3 was expressed in differentiating cells coincident with profilaggrin and keratin 10 expressions (Hitomi et al, 2003).

Disheveled associated activator of morphogenesis 2. Disheveled associated activator of morphogenesis 2 (DAAM2, KIAA0381, MGC90515, dJ90A20A.1, RP1-278E11.1) regulates the morphogenetic movements of vertebrate gastrulation in a Wnt-dependent manner through direct interactions with Dsh/Dvl and RhoA (Nakaya et al, 2004). The observed expression patterns in developing central nervous tissues suggested that vertebrate Daam genes were involved in pivotal steps in neuronal cell differentiation and movement (Kida et al, 2004).

OTTHUMG00000030521, AC000095.4. OTTHUMG00000030521, AC000095.4 (Vega gene ID) alias Em:AC000095.C22.4 putative processed transcript is probably DiGeorge Syndrome gene B (Vega gene Report).

CAP-binding protein complex interacting protein 1 isoform. CAP-binding protein complex interacting protein 1 isoform (a Source: RefSeq_peptide NP_{—}073622) alias FLJ23588; DJBP; HSCBCIP1; KIAA1672; dJ185D5.1). DJBP mRNA was found to be specifically expressed in the testis. In addition to the binding of DJBP to the COOH-terminal region of DJ-1, DJBP was also found to bind in vitro and in vivo to the DNA-binding domain of the androgen receptor (AR) in a testosterone-dependent manner and to be colocalized with DJ-1 or AR in the nucleus. Furthermore, a co-immunoprecipitation assay showed that the formation of a ternary complex between DJ-1, DJBP, and AR occurred in cells in which DJ-1 bound to the AR via DJBP. It was found that DJBP repressed a testosterone-dependent AR transactivation activity in monkey Cos1 cells by recruiting histone deacetylase (HDAC) complex (Niki et al, 2003). Necropsy tissues from 11 cases were analyzed with 1 tumor specimen found to have HIV integrated in chromosome 22q13.2 and within 300 kb of HSCBCIP1 (CAP-binding protein complex interacting homologue). Tumor-specific primers were then used to screen uninvolved tissue from the same patient, which did not amplify the site-specific region (Killebrew et al, 2004).

T-box 18. T-box 18 (TBX18). Forty-four transcripts with expression differences higher than 2-fold (T test, P< or =0.05) were detected between forelimb and hindlimb tissues including 38 new transcripts such as Rdh10, Frzb, Tbx18, and Hip that exhibit differential limb expression (Shou et al, 2005). T-box genes have been implicated in early cardiac lineage determination, chamber specification, valvuloseptal development, and diversification of the specialized conduction system in vertebrate embryos. These genes include Tbx1, Tbx2, Tbx3, Tbx5, Tbx18, and Tbx20, all of which exhibit complex temporal spatial regulation in developing cardiac structures (Plageman and Yutzey, 2005). It was demonstrated that maintenance of anterior-posterior-somite polarity is mediated by the T-box transcription factor Tbx18 (Bussen et al, 2004). Given the haploinsufficiency phenotypes reported for other T-box genes, we speculate that allelic imbalance (AI) may influence the function of Tbx18 during osteosarcomagenesis (Rosemann et al, 2004).

PLA2G3. PLA2G3 is also known as phospholipase A2 group III (GIII-SPLA2, sPLA(2)-III). Human group III secreted phospholipase A(2) (sPLA(2)-III) consists of a central group III sPLA(2) domain flanked by unique N- and C-terminal domains. It was found that the sPLA(2) domain alone was sufficient for its catalytic activity and for its prostaglandin E(2) (PGE(2))-generating functions in various cell types. Immunohistochemistry demonstrated that sPLA(2)-III was preferentially expressed in the microvascular endothelium in human tissues with inflammation, ischemic injury, and cancer. In support of this, sPLA(2)-III was induced in cultured microvascular endothelial cells after stimulation with proinflammatory cytokines. Expression of sPLA(2)-III was also associated with various tumor cells, and colorectal cancer cells transfected with sPLA(2)-III exhibited enhanced PGE(2) production and cell proliferation, which required sPLA(2)-III catalytic activity (Murakami et al, 2005).

OTTHUMG00000030140. OTTHUMG00000030140, CTA-299D3.6 (Vega gene ID) alias bA262A13.C22.5. Identification of the breakpoint between CTA-299D3 and RP5-925J7 probe, located in 22q13.32. Deletion extent could be estimated to be about 2.5 Mb in a patient with ring chromosome 22 (includes mental retardation with severe language impairment, hypotonia, and dysmorphic facial features) (Battini et al, 2004).

Solute carrier family 7 (cationic amino acid transporter, y+ system) member 4. Solute carrier family 7 (cationic amino acid transporter, y+ system) member 4 (SLC7A4, CAT4, CAT-4, HCAT3, MGC129976, MGC129977) exhibits significant sequence homology with the SLC7 subfamily of human cationic amino acid transporters (hCATs). Human glioblastoma cells stably overexpressing a fusion protein between SLC7A4 and the enhanced green fluorescent protein (EGFP) did not exhibit an increased transport activity for 1-arginine. The lack of transport activity was not due to a lack of SLC7A4 protein expression in the plasma membrane. The expression of SLC7A4 can be induced in NT2 teratocarcinoma cells by treatment with retinoic acid. However, also for this endogenously expressed SLC7A4, any transport activity for 1-arginine could not be detected. Therefore, SLC7A4 is either not an amino acid transporter or it needs additional (protein) factor(s) to be functional (Wolf et al, 2002). SLC7A4 was mapped to 22q11.2, the commonly deleted region of the velocardiofacial syndrome (VCFS, Shprintzen syndrome). In a patient affected by VCFS, deletion of SLC7A4 was demonstrated by chromosomal FISH. By Northern analysis, an abundant transcript was detected in brain, testis, and placenta (Sperandeo et al, 1998).

Sushi domain containing 2. Sushi domain containing 2 (SUSD2 alias BK65A6.2, FLJ22778). Neuronal marker proteins are widely used for characterization and identification of normal and tumor tissue of the central nervous system, but the most commonly used neuronal markers have inherent methodological problems. A proteomic approach was used comprising two-dimensional (2-D) gel electrophoresis and subsequent MALDI identification to identify possible new marker proteins in the human cortical neuronal cell line HCN-2. 14 proteins were found, among them BK65A6.2 (Novel Sushi Domain (Scr repeat)) (Peyrl et al, 2003).

Phosphatidylinositol (4,5) bisphosphate 5-phosphatase, A. Phosphatidylinositol (4,5) bisphosphate 5-phosphatase A (PIB5PA alias PIPP; INPP5; MGC129984 PIPP) hydrolyzes PtdIns(3,4,5)P3 forming PtdIns(3,4)P2, decreasing Ser473-Akt phosphorylation. PIPP is expressed in PC12 cells, localizing to the plasma membrane of undifferentiated cells and the neurite shaft and growth cone of NGF-differentiated neurites. Overexpression of wild-type, but not catalytically-inactive PIPP, in PC12 cells inhibited neurite elongation. Targeted depletion of PIPP using RNA interference (RNAi) resulted in enhanced neurite differentiation, associated with neurite hyper-elongation (Ooms et al 2005).

Signal peptide-CUB domian-EGF-related 1. Signal peptide-CUB domian-EGF-related 1 (SCUBE1) mRNA is found in several highly vascularized tissues such as liver, kidney, lung, spleen, and brain and is selectively expressed in vascular endothelial cells (EC) by in situ hybridization (Yang et al, 2002). The Scube1 (signal peptide-CUB domain-EGF-related 1) gene was isolated from a developing mouse urogenital ridge cDNA library and is expressed prominently in the developing gonad, nervous system, somites, surface ectoderm, and limb buds (Grimmond et al, 2000). SCUBE1 and SCUBE2 define an emerging family of human secreted proteins that are expressed in vascular endothelium and may play important roles in development, inflammation, and thrombosis (Yang et al, 2000).

RP3-355C18.2 (Vega gene ID). RP3-355C18.2 (Vega gene ID) alias dJ355C18.C22.2 is similar to AK023960 (according to Vega Report).

RP1-47A17.8 (Vega gene ID). RP1-47A17.8 (Vega gene ID) alias dJ47A17.C22.8 is similar to AL110226 cDNA DKFZp434H204 (According to Vega Report).

KB-1323B2.3. Embryonic marker KB-1323B2.3 alias Em:AP000557.C22.3 is similar to Em:AF012872 human phosphatidylinositol 4-kinase 230 (According to Vega Report).

Breast carcinoma amplified sequence 4. The breast carcinoma amplified sequence 4 (BCAS4 alias FLJ20495, BHLHB4) gene at 20q13.2 encodes a 211 amino acid cytoplasmic protein. In the MCF7 breast cancer cell line, the BCAS3 and BCAS4 genes were co-amplified, and cloning of a highly overexpressed 1.3-kb transcript revealed a rearrangement fusing the last two exons of BCAS3 with BCAS4. The BCAS4-BCAS3 fusion transcript was detected only in MCF7 cells, but the BCAS4 gene was also overexpressed in nine of 13 breast cancer cell lines (Barlund et al, 2004).

SAM50-like protein CGI-51. SAM50-like protein CGI-51 also known as SAM50_HUMAN (UniProt/Swiss-Prot ID) or sorting and assembly machinery component 50 homolog (S. Cerevisiae) (SAMM50 alias OMP85, SAM50, TOB55, TRG-3, CGI-51, YNL026W). Tob55 is an essential component of the TOB complex in that it constitutes the core element of the protein-conducting pore. The other two components of the TOB complex are Tob38, which builds a functional TOB core complex with Tob55, and Mas37, a peripheral member of the complex. Reduced insertion of the Tob55 precursor in the absence of Tom20 and Tom70 argues for initial recognition of the precursor of Tob55 by the import receptors. Next, it is transferred through the import channel formed by Tom40 (Habib et al, 2005).

TPX1_testis specific protein 1. TPX1_testis specific protein 1 (probe H4-1 p3-1) (CRISP2 alias TPX1, TSP1, GAPDL5, CRISP-2, MGC111136). TPX1 is a component of the sperm acrosome that remains associated with sperm after capacitation and acrosome reaction, and is relevant for sperm-oocyte interaction (Busso et al, 2004). There is significant difference in the expression levels of TPX-1 between normal (n=29) and motility impaired (n=24) semen samples, indicating that this gene is involved in sperm function (Wang et al, 2004). RT-PCR analysis of RNA isolated from acinar cells of lacrimal glands revealed that they expressed CRISP-1 and CRISP-2 (Haendler et al, 1999).

Nesprin-1. Nesprin-1 (Nuclear envelope spectrin repeat protein 1) is also known as synaptic nuclear envelope protein 1 (Syne-1), Myocyte nuclear envelope protein 1 (Myne-1) or Enaptin (SYNE1 alias SYNE-1B, KIAA0796, 8B, nesprin-1, enaptin, MYNE1, CPG2). Transgenic mice overexpressing the conserved C-terminal Klarsicht/ANC-1/Syne homology domain of Syne-1 were generated. The transgene acted in a dominant interfering fashion, displacing endogenous Syne-1 from the nuclear envelope. Muscle nuclei failed to aggregate at the NMJ in transgenic mice, demonstrating that localization and positioning of synaptic nuclei require Syne proteins (Grady et al, 2005). Integral membrane protein nesprin-1alpha serves as a receptor for mAKAP on the nuclear envelope in cardiac myocytes (Pare et al, 2005). Syne-1 and KIF3B function together in cytokinesis by facilitating the accumulation of membrane vesicles at the spindle midbody (Fan and Beck, 2004). Syne-1 gene is expressed in a variety of forms that are multifunctional and are capable of functioning at both the Golgi and the nuclear envelope, perhaps linking the two organelles during muscle differentiation (Gough et al, 2003). Nesprin-1 is developmentally regulated in both smooth and skeletal muscle and is re-localized from the nuclear envelope to the nucleus and cytoplasm during C2C12 myoblast differentiation (Zhang et al, 2001).

FLOT1. FLOT1 is also known as flotillin or 1 ENSG00000137312. Caveolae are small domains on the inner cell membrane involved in vesicular trafficking and signal transduction. FLOT1 encodes a caveolae-associated, integral membrane protein. The function of flotillin 1 has not been determined (Entrez database).

C6 orf25. C6orf25 is also known as chromosome 6 open reading frame 25; ENSG00000096148; C6orf25; G6b; NG31. This gene is a member of the immunoglobulin (Ig) superfamily and is located in the major histocompatibility complex (MHC) class III region. The protein encoded by this gene is a glycosylated, plasma membrane-bound cell surface receptor, but soluble isoforms encoded by some transcript variants have been found in the endoplasmic reticulum and Golgi before being secreted. Seven transcript variants encoding different isoforms have been described for this gene (Entrez database).

VARS. VARS is also known as valyl-tRNA synthetase; ENSG00000096171; G7A or VARS2.

Aminoacyl-tRNA synthetases catalyze the aminoacylation of tRNA by their cognate amino acid. Because of their central role in linking amino acids with nucleotide triplets contained in tRNAs, aminoacyl-tRNA synthetases are thought to be among the first proteins that appeared in evolution. The protein encoded by this gene belongs to class-I aminoacyl-tRNA synthetase family and is located in the class III region of the major histocompatibility complex (Entrez database).

Major histocompatibility complex, class II, DP beta 1. Major histocompatibility complex, class II, DP beta 1 is also known as OTTHUMG00000031076; HLA-DPB1; HLA-DPB1; DPB1; HLA-DP1 B; MHC DPB1. HLA-DPB belongs to the HLA class II beta chain paralogues. This class II molecule is a heterodimer consisting of an alpha (DPA) and a beta chain (DPB), both anchored in the membrane. It plays a central role in the immune system by presenting peptides derived from extracellular proteins. Class II molecules are expressed in antigen presenting cells (APC: B lymphocytes, dendritic cells, macrophages). The beta chain is approximately 26-28 kDa and its gene contains 6 exons. Exon one encodes the leader peptide, exons 2 and 3 encode the two extracellular domains, exon 4 encodes the transmembrane domain and exon 5 encodes the cytoplasmic tail. Within the DP molecule both the alpha chain and the beta chain contain the polymorphisms specifying the peptide binding specificities, resulting in up to 4 different molecules (Entrez database).

Major histocompatibility complex, class II, DR beta 5. Major histocompatibility complex, class II, DR beta 5 is also known as OTTHUMG00000031027; HLA-DRB5. HLA-DRB5 belongs to the HLA class II beta chain paralogues. This class II molecule is a heterodimer consisting of an alpha (DRA) and a beta (DRB) chain, both anchored in the membrane. It plays a central role in the immune system by presenting peptides derived from extracellular proteins. Class II molecules are expressed in antigen presenting cells (APC: B lymphocytes, dendritic cells, macrophages). The beta chain is approximately 26-28 kDa and its gene contains 6 exons. Exon one encodes the leader peptide, exons 2 and 3 encode the two extracellular domains, exon 4 encodes the transmembrane domain and exon 5 encodes the cytoplasmic tail. Within the DR molecule the beta chain contains all the polymorphisms specifying the peptide binding specificities. Typing for these polymorphisms is routinely done for bone marrow and kidney transplantation. DRB1 is expressed at a level five times higher than its paralogues DRB3, DRB4 and DRB5. The presence of DRB5 is linked with allelic variants of DRB1, otherwise it is omitted. There are 4 related pseudogenes: DRB2, DRB6, DRB7, DRB8 and DRB9 (Entrez database).

COL11A2. COL11A2 is also known as collagen, type XI, alpha 2; OTTHUMG00000031036; HKE5; PARP; STL3; DFNA13; DFNB53. This gene encodes one of the two alpha chains of type XI collagen, a minor fibrillar collagen. It is located on chromosome 6 very close to but separate from the gene for retinoid X receptor beta. Type XI collagen is a heterotrimer but the third alpha chain is a post-translationally modified alpha 1 type II chain. Proteolytic processing of this type XI chain produces PARP, a proline/arginine-rich protein that is an amino terminal domain. Mutations in this gene are associated with type III Stickler syndrome, otospondylomegaepiphyseal dysplasia (OSMED syndrome), Weissenbacher-Zweymuller syndrome, and autosomal dominant nonsyndromic sensorineural 13 deafness. Three transcript variants encoding different isoforms have been identified for this gene (Entrez database).

PRAME. PRAME is also known as melanoma antigen preferentially expressed in tumors; preferentially expressed antigen of melanoma or OPA-interacting protein 4 (OIP4 alias ENSG0000185686; MAPE). This gene encodes an antigen that is predominantly expressed in human melanomas and that is recognized by cytolytic T lymphocytes. It is not expressed in normal tissues, except testis. This expression pattern is similar to that of other CT antigens, such as MAGE, BAGE and GAGE. However, unlike these other CT antigens, this gene is also expressed in acute leukemias. Five alternatively spliced transcript variants encoding the same protein have been observed for this gene (Entrez database).

FBLN1. FBLN1 is also known as fibulin 1 or ENSG00000077942. Fibulin 1 is a secreted glycoprotein that becomes incorporated into a fibrillar extracellular matrix. Calcium-binding is apparently required to mediate its binding to laminin and nidogen. It mediates platelet adhesion via binding fibrinogen. Four splice variants which differ in the 3′ end have been identified. Each variant encodes a different isoform, but no functional distinctions have been identified among the four variants (Entrez database).

CYP2D6. CYP2D6 is also known as cytochrome P450, family 2, subfamily D, polypeptide 6 or ENSG00000100197 (alias CPD6; CYP2D; CYP2D@; CYP2DL1; P450C2D; P450-DB1; MGC120389; MGC120390). This gene encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum and is known to metabolize as many as 20% of commonly prescribed drugs. Its substrates include debrisoquine, an adrenergic-blocking drug; sparteine and propafenone, both anti-arrythmic drugs; and amitryptiline, an anti-depressant. The gene is highly polymorphic in the population; certain alleles result in the poor metabolizer phenotype, characterized by a decreased ability to metabolize the enzyme's substrates. The gene is located near two cytochrome P450 pseudogenes on chromosome 22q13.1. Alternatively spliced transcript variants encoding different isoforms have been found for this gene (Entrez database).

AC006548.8 (Vega gene ID). AC006548.8 (Vega gene ID) is also known as Em:AC006548.C22.8. This gene is similar to Em:AB018413 human Gab2 (According to Vega Report).

Homo sapiens cat eye syndrome critical region 3 (CECR3) gene. Homo sapiens cat eye syndrome critical region 3 (CECR3) gene (According to Vega Report) is also known as topoisomerase (DNA) III beta, OTTHUMG00000030764 or TOP3B. This gene encodes a DNA topoisomerase, an enzyme that controls and alters the topologic states of DNA during transcription. This enzyme catalyzes the transient breaking and rejoining of a single strand of DNA which allows the strands to pass through one another, thus relaxing the supercoils and altering the topology of DNA. The enzyme interacts with DNA helicase SGS1 and plays a role in DNA recombination, cellular aging and maintenance of genome stability. Alternative splicing of the C-terminus of this gene results in three transcript variants which have distinct tissue specificity; however, not all variants have been fully described (Entrez database).

KB-1269D1.3 (Vega gene ID). KB-1269D1.3 (Vega gene ID) is also known as OTTHUMG00000030694 or Em:AP000344.C22.3. It is similar to Tr:Q13312 human TXBP181 (According to Vega Report).

GPR24. GPR24 is also known as G protein-coupled receptor 24; ENSG00000128285; SLC1; MCHR1; MGC32129. The protein encoded by this gene, a member of the G protein-coupled receptor family 1, is an integral plasma membrane protein which binds melanin-concentrating hormone. The encoded protein can inhibit cAMP accumulation and stimulate intracellular calcium flux, and is probably involved in the neuronal regulation of food consumption. Although structurally similar to somatostatin receptors, this protein does not seem to bind somatostatin (Entrez database).

GAL3ST1 GAL3ST1 is also known as galactose-3-O-sulfotransferase 1; ENSG00000128242; CST. Sulfonation, an important step in the metabolism of many drugs, xenobiotics, hormones, and neurotransmitters, is catalyzed by sulfotransferases. The product of this gene is galactosylceramide sulfotransferase which catalyzes the conversion between 3′-phosphoadenylylsulfate+a galactosylceramide to adenosine 3′,5′-bisphosphate+galactosylceramide sulfate. Activity of this sulfotransferase is enhanced in renal cell carcinoma (Entrez database).

GSTT3-3 similar to Glutathione S-transferases (according to Vega Report). GSTT3-3 similar to Glutathione S-transferases (according to Vega Report) is also known as RP4-539M6.7 (Vega gene ID); OTTHUMG00000030918 or Em:AC004832.C22.7. It is similar to TR:Q9Y2Z7 Homo sapiens CGI-08 PROTEIN (according to Vega Report).

GALR3GALR3 is also known as galanin receptor 3 or ENSG00000128310. The neuropeptide galanin modulates a variety of physiologic processes including cognition/memory, sensory/pain processing, hormone secretion, and feeding behavior. The human galanin receptors are G protein-coupled receptors that functionally couple to their intracellular effector through distinct signaling pathways. GALR3 is found in many tissues and may be expressed as 1.4-, 2.4-, and 5-kb transcripts (Entrez database).

IL2RB. IL2RB is also known as interleukin 2 receptor, beta; ENSG00000100385 or P70-75. The interleukin 2 receptor, which is involved in T cell-mediated immune responses, is present in 3 forms with respect to ability to bind interleukin 2. The low affinity form is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. The protein encoded by this gene represents the beta subunit and is a type I membrane protein (Entrez database).

DGCR2. DGCR2 is also known as DiGeorge syndrome critical region gene 2; ENSG00000070413; IDD; LAN; DGS-C; SEZ-12; KIAA0163; DKFZp68611730. Deletions of the 22q11.2 have been associated with a wide range of developmental defects (notably DiGeorge syndrome, velocardiofacial syndrome, conotruncal anomaly face syndrome and isolated conotruncal cardiac defects) classified under the acronym CATCH 22. The DGCR2 gene encodes a novel putative adhesion receptor protein, which could play a role in neural crest cells migration, a process which has been proposed to be altered in DiGeorge syndrome (Entrez database).

TCN2. TCN2 is also known as transcobalamin II; macrocytic anemia; ENSG00000185339 (alias TC2; D22S676; D22S750). This gene encodes a member of the vitamin B12-binding protein family. This family of proteins, alternatively referred to as R binders, is expressed in various tissues and secretions. This plasma protein binds cobalamin and mediates the transport of cobalamin into cells. This protein and other mammalian cobalamin-binding proteins, such as transcobalamin I and gastric intrisic factor, may have evolved by duplication of a common ancestral gene (Entrez database).

IGLL1. IGLL1 is also known as immunoglobulin lambda-like polypeptide 1 or ENSG00000128322 (alias IGO; 14.1; IGL1; IGL5; IGLL; IGVPB; CD179b; VPREB2; IGLJ14.1). The preB cell receptor is found on the surface of proB and preB cells, where it is involved in transduction of signals for cellular proliferation, differentiation from the proB cell to the preB cell stage, allelic exclusion at the Ig heavy chain gene locus, and promotion of Ig light chain gene rearrangements. The preB cell receptor is composed of a membrane-bound Ig mu heavy chain in association with a heterodimeric surrogate light chain. This gene encodes one of the surrogate light chain subunits and is a member of the immunoglobulin gene superfamily. This gene does not undergo rearrangement. Mutations in this gene can result in B cell deficiency and agammaglobulinemia, an autosomal recessive disease in which few or no gamma globulins or antibodies are made. Two transcript variants encoding different isoforms have been found for this gene (Entrez database).

APOBEC3B. APOBEC3B is also known as apolipoprotein B mRNA editing enzyme, catalytic polypeptide-like 3B; ENSG00000179750 (alias ARP4; ARCD3; PHRBNL; APOBEC1L; FLJ21201; DJ742C19.2). This gene is a member of the cytidine deaminase gene family. It is one of seven related genes or pseudogenes found in a cluster, thought to result from gene duplication, on chromosome 22. Members of the cluster encode proteins that are structurally and functionally related to the C to U RNA-editing cytidine deaminase APOBEC1. It is thought that the proteins may be RNA editing enzymes and have roles in growth or cell cycle control (Entrez database).

CRYBB1. CRYBB1 is also known as crystallin, beta B1 or ENSG00000100122. Crystallins are separated into two classes: taxon-specific, or enzyme, and ubiquitous. The latter class constitutes the major proteins of vertebrate eye lens and maintains the transparency and refractive index of the lens. Since lens central fiber cells lose their nuclei during development, these crystallins are made and then retained throughout life, making them extremely stable proteins. Mammalian lens crystallins are divided into alpha, beta, and gamma families; beta and gamma crystallins are also considered as a superfamily. Alpha and beta families are further divided into acidic and basic groups. Seven protein regions exist in crystallins: four homologous motifs, a connecting peptide, and N- and C-terminal extensions. Beta-crystallins, the most heterogeneous, differ by the presence of the C-terminal extension (present in the basic group, none in the acidic group). Beta-crystallins form aggregates of different sizes and are able to self-associate to form dimers or to form heterodimers with other beta-crystallins. This gene, a beta basic group member, undergoes extensive cleavage at its N-terminal extension during lens maturation. It is also a member of a gene cluster with beta-A4, beta-B2, and beta-B3 (Entrez database).

CRYBA4. CRYBA4 is also known as crystallin, beta A4 or ENSG00000196431. Crystallins are separated into two classes: taxon-specific, or enzyme, and ubiquitous. The latter class constitutes the major proteins of vertebrate eye lens and maintains the transparency and refractive index of the lens. Since lens central fiber cells lose their nuclei during development, these crystallins are made and then retained throughout life, making them extremely stable proteins. Mammalian lens crystallins are divided into alpha, beta, and gamma families; beta and gamma crystallins are also considered as a superfamily. Alpha and beta families are further divided into acidic and basic groups. Seven protein regions exist in crystallins: four homologous motifs, a connecting peptide, and N- and C-terminal extensions. Beta-crystallins, the most heterogeneous, differ by the presence of the C-terminal extension (present in the basic group, none in the acidic group). Beta-crystallins form aggregates of different sizes and are able to self-associate to form dimers or to form heterodimers with other beta-crystallins. This gene, a beta acidic group member, is part of a gene cluster with beta-B1, beta-B2, and beta-B3 (Entrez database).

APOL4. APOL4 is also known as apolipoprotein L 4 or ENSG00000100336 (alias APOLIV; APOL-IV). The protein encoded by this gene is a member of the apolipoprotein L family and may play a role in lipid exchange and transport throughout the body, as well as in reverse cholesterol transport from peripheral cells to the liver. Two transcript variants encoding two different isoforms have been found for this gene. Only one of the isoforms appears to be a secreted protein (Entrez database).

SOX10. SOX10 is also known as SRY (sex determining region Y)-box 10 or ENSG00000100146 (alias DOM; WS4; MGC15649). This gene encodes a member of the SOX (SRY-related HMG-box) family of transcription factors involved in the regulation of embryonic development and in the determination of the cell fate. The encoded protein may act as a transcriptional activator after forming a protein complex with other proteins. This protein acts as a nucleocytoplasmic shuttle protein and is important for neural crest and peripheral nervous system development. Mutations in this gene are associated with Waardenburg-Shah and Waardenburg-Hirschsprung disease (Entrez database).

MGAT3. MGAT3 is also known as mannosyl (beta-1,4-)-glycoprotein beta-1,4-N-acetylglucosaminyltransferase or ENSG00000128268 (alias GNT3; GNT-III). There are believed to be over 100 different glycosyltransferases involved in the synthesis of protein-bound and lipid-bound oligosaccharides. N-acetylglucosaminyltransferase III transfers a GlcNAc residue to the beta-linked mannose of the trimannosyl core of N-linked oligosaccharides and produces a bisecting GlcNAc. Expression of this gene may be controlled by a multiple-promoter system (Entrez database).

RABL4. RABL4 is also known as RAB member of RAS oncogene family-like 4 or ENSG00000100360 (alias RAYL). This gene encodes a putative GTP-binding protein similar to RAY/RAB1C. The protein is ras-related, but the function is unknown (Entrez database).

SULT4A1. SULT4A1 is also known as sulfotransferase family 4A, member 1; or ENSG00000130540 (alias NST; BRSTL1; SULTX3; BR-STL-1; MGC40032; DJ388M5.3; hBR-STL-1). Sulfotransferase enzymes catalyze the sulfate conjugation of many hormones, neurotransmitters, drugs, and xenobiotic compounds. These cytosolic enzymes are different in their tissue distributions and substrate specificities. The gene structure (number and length of exons) is similar among family members. This gene encodes a protein that is selectively expressed in brain tissue (Entrez database).

RPL3. RPL3 is also known as ribosomal protein L3 or ENSG00000100316 alias (TARBP-B; MGC104284). Ribosomes, the complexes that catalyze protein synthesis, consist of a small 40S subunit and a large 60S subunit. Together these subunits are composed of 4 RNA species and approximately 80 structurally distinct proteins. This gene encodes a ribosomal protein that is a component of the 60S subunit. The protein belongs to the L3P family of ribosomal proteins and it is located in the cytoplasm. The protein can bind to the HIV-1 TAR mRNA, and it has been suggested that the protein contributes to tat-mediated transactivation. This gene is co-transcribed with several small nucleolar RNA genes, which are located in several introns of this gene. Alternate transcriptional splice variants, encoding different isoforms, have been characterized. As is typical for genes encoding ribosomal proteins, there are multiple processed pseudogenes of this gene dispersed through the genome (Entrez database).

APOL2. APOL2 is also known as apolipoprotein L, 2 or ENSG00000128335 (alias APOL-II). This gene is a member of the apolipoprotein L gene family. The encoded protein is found in the cytoplasm, where it may affect the movement of lipids or allow the binding of lipids to organelles. Two transcript variants encoding the same protein have been found for this gene (Entrez database).

RAC2. RAC2 is also known as ras-related C3 botulinum toxin substrate 2 (rho family, small GTP binding protein Rac2) or ENSG00000128340 (alias Gx; EN-7; HSPC022). The protein encoded by this gene is a GTPase which belongs to the RAS superfamily of small GTP-binding proteins. Members of this superfamily appear to regulate a diverse array of cellular events, including the control of cell growth, cytoskeletal reorganization, and the activation of protein kinases (Entrez database).

GABRR2. GABRR2 is also known as gamma-aminobutyric acid (GABA) receptor, rho 2 or ENSG00000111886. GABA is the major inhibitory neurotransmitter in the mammalian brain where it acts at GABA receptors, which are ligand-gated chloride channels. GABRR2 is a member of the rho subunit family (Entrez database).

MOG. MOG is also known as myelin oligodendrocyte glycoprotein or ENSG00000137345 (alias MGC26137). The product of this gene is a membrane protein expressed on the oligodendrocyte cell surface and the outermost surface of myelin sheaths. Due to this localization, it is a primary target antigen involved in immune-mediated demyelination. This protein may be involved in completion and maintenance of the myelin sheath and in cell-cell communication. Alternatively spliced transcript variants encoding different isoforms have been identified (Entrez database).

ME1. ME1 is also known as malic enzyme 1, NADP(+)-dependent, cytosolic or ENSG00000065833 (alias MES; HUMNDME). This gene encodes a cytosolic, NADP-dependent enzyme that generates NADPH for fatty acid biosynthesis. The activity of this enzyme, the reversible oxidative decarboxylation of malate, links the glycolytic and citric acid cycles. The regulation of expression for this gene is complex. Increased expression can result from elevated levels of thyroid hormones or by higher proportions of carbohydrates in the diet (Entrez database).

IL20RA. IL20RA is also known as interleukin 20 receptor, alpha or ENSG00000016402 (alias IL-20R1; ZCYTOR7). The protein encoded by this gene is a receptor for interleukin 20 (IL20), a cytokine that may be involved in epidermal function. The receptor of IL20 is a heterodimeric receptor complex consisting of this protein and interleukin 20 receptor beta (IL20B). This gene and IL20B are highly expressed in skin. The expression of both genes is found to be upregulated in Psoriasis (Entrez database).

ZHX3. ZHX3 is also known as zinc fingers and homeoboxes 3 or OTTHUMG00000032481 (alias TIX1; KIAA0395). This gene encodes a member of the zinc fingers and homeoboxes (ZHX) gene family. The encoded protein contains two C2H2-type zinc fingers and five homeodomains and forms a dimer with itself or with zinc fingers and homeoboxes family member 1. In the nucleus, the dimerized protein interacts with the A subunit of the ubiquitous transcription factor nuclear factor-Y and may function as a transcriptional repressor (Entrez database).

CHD6. CHD6 is also known as chromodomain helicase DNA binding protein 6 or ENSG00000124177 (alias CHD5; RIGB; KIAA1335). Chromosomal DNA of eukaryotic cells is compacted by nuclear proteins to form chromatin, an organized nucleoprotein structure that can inhibit gene expression. Several multisubunit protein complexes exist to remodel the chromatin to allow patterns of cell type-specific gene expression. The protein encoded by this gene is thought to be a core member of one or more of these complexes. The encoded protein, which is a member of the SNF2/RAD54 helicase family, contains two chromodomains, a helicase domain, and an ATPase domain (Entrez database).

PTPRG. PTPRG is also known as protein tyrosine phosphatase, receptor type, 6 or ENSG00000144724 (alias PTPG; HPTPG; RPTPG; R-PTP-GAMMA). The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP possesses an extracellular region, a single transmembrane region, and two tandem intracytoplasmic catalytic domains, and thus represents a receptor-type PTP. The extracellular region of this PTP contains a carbonic anhydrase-like (CAH) domain, which is also found in the extracellular region of PTPRBETA/ZETA. This gene is located in a chromosomal region that is frequently deleted in renal cell carcinoma and lung carcinoma, thus is thought to be a candidate tumor suppressor gene (Entrez database).

PTPNS1. PTPNS1 is also known as protein tyrosine phosphatase, non-receptor type substrate 1 or ENSG00000198053 (alias BIT; MFR; P84; SIRP; MYD-1; SHPS1; SHPS-1; SIRPalpha; SIRPalpha2; SIRP-ALPHA-1). The protein encoded by this gene is a member of the signal-regulatory-protein (SIRP) family, and also belongs to the immunoglobulin superfamily. SIRP family members are receptor-type transmembrane glycoproteins known to be involved in the negative regulation of receptor tyrosine kinase-coupled signaling processes. This protein can be phosphorylated by tyrosine kinases. The phospho-tyrosine residues of this PTP have been shown to recruit SH2 domain containing tyrosine phosphatases (PTP), and serve as substrates of PTPs. This protein was found to participate in signal transduction mediated by various growth factor receptors. CD47 has been demonstrated to be a ligand for this receptor protein. This gene and its product share very high similarity with several other members of the SIRP family. These related genes are located in close proximity to each other on chromosome 20p13 (Entrez database).

PCSK2. PCSK2 is also known as proprotein convertase subtilisin/kexin type 2 or ENSG00000125851 (alias PC2; NEC2; SPC2). The protein encoded by this gene belongs to the subtilisin-like proprotein convertase family. The members of this family are proprotein convertases that process latent precursor proteins into their biologically active products. This encoded protein is a proinsulin-processing enzyme that plays a key role in regulating insulin biosynthesis. It is also known to cleave proopiomelanocortin, proenkephalin, prodynorphin and proluteinizing-hormone-releasing hormone. The use of alternate polyadenylation sites has been found for this gene (Entrez database).

PLAGL2. PLAGL2 is also known as pleiomorphic adenoma gene-like 2 or ENSG00000126003. It is a zinc-finger protein that recognizes DNA and/or RNA (Entrez database).

GGTL3. GGTL3 is also known as gamma-glutamyltransferase-like 3 or ENSG00000131067 (alias GGTL5; D20S101; dJ18C9.2). Gamma-glutamyltransferase is a membrane-associated protein involved in both the metabolism of glutathione and in the transpeptidation of amino acids. Changes in the activity of gamma-glutamyltransferase may signal preneoplastic or toxic conditions in the liver or kidney. The protein encoded by this gene is similar in sequence to gamma-glutamyltransferase, but its function is unknown. Multiple transcript variants encoding several different isoforms have been found for this gene (Entrez database).

EPB41L1. EPB41L1 is also known as erythrocyte membrane protein band 4.1-like 1 or ENSG00000088367 (alias KIAA0338; MGC11072). Erythrocyte membrane protein band 4.1 (EPB41) is a multifunctional protein that mediates interactions between the erythrocyte cytoskeleton and the overlying plasma membrane. The protein encoded by this gene is a neuronally-enriched protein that is structurally similar to EPB41. The encoded protein binds and stabilizes D2 and D3 dopamine receptors at the neuronal plasma membrane. Multiple transcript variants encoding different isoforms have been found for this gene, but the full-length nature of only two of them has been determined (Entrez database).

SDC4. SDC4 is also known as syndecan 4 (amphighlycan, ryudocan) or ENSG00000124145 (alias SYND4; MGC22217). The protein encoded by this gene is a transmembrane (type 1) heparan sulfate proteoglycan that functions as a receptor in intracellular signaling. The encoded protein is found as a homodimer and is a member of the syndecan proteoglycan family. This gene is found on chromosome 20, while a pseudogene has been found on chromosome 22 (Entrez database).

EYA2. EYA2 is also known as eyes absent homolog 2 (Drosophila) or ENSG00000064655 (alias EAB1; MGC10614). This gene encodes a member of the eyes absent (EYA) family of proteins. The encoded protein may be post-translationally modified and may play a role in eye development. A similar protein in mice can act as a transcriptional activator. Five transcript variants encoding three distinct isoforms have been identified for this gene (Entrez database).

USP18. USP18 is also known as ubiquitin specific peptidase 18 or OTTHUMG00000030949 (alias ISG43; UBP43). USP18, a member of the deubiquitinating protease family of enzymes, removes ubiquitin adducts from a broad range of protein substrates (supplied by OMIM).

BCR. BCR is also known as breakpoint cluster region or ENSG00000186716 (alias ALL; CML; PHL; BCR1; D22S11; D22S662). A reciprocal translocation between chromosomes 22 and 9 produces the Philadelphia chromosome, which is often found in patients with chronic myelogenous leukemia. The chromosome 22 breakpoint for this translocation is located within the BCR gene. The translocation produces a fusion protein which is encoded by sequence from both BCR and ABL, the gene at the chromosome 9 breakpoint. Although the BCR-ABL fusion protein has been extensively studied, the function of the normal BCR gene product is not clear. The protein has serine/threonine kinase activity and is a GTPase-activating protein for p21rac. Two transcript variants encoding different isoforms have been found for this gene (Entrez database).

MAPK8IP2. MAPK8IP2 is also known as mitogen-activated protein kinase 8 interacting protein 2 or ENSG00000008735 (alias IB2; JIP2; PRKM8IPL). The protein encoded by this gene is closely related to MAPK8IP1/IB1/JIP-1, a scaffold protein that is involved in the c-Jun amino-terminal kinase signaling pathway. This protein is expressed in brain and pancreatic cells. It has been shown to interact with, and regulate the activity of MAPK8/JNK1, and MAP2K7/MKK7 kinases. This protein thus is thought to function as a regulator of signal transduction by protein kinase cascade in brain and pancreatic beta-cells. Three alternatively spliced transcript variants of this gene encoding distinct isoforms have been reported (Entrez database).

MN1. MN1 is also known as meningioma (disrupted in balanced translocation) 1 or ENSG00000169184 (alias MGCR; MGCR1; MGCR1-PEN; dJ353E16.2). Meningioma 1 (MN1) contains two sets of CAG repeats. It is disrupted by a balanced translocation (4;22) in a meningioma, and its inactivation may contribute to meningioma 32 pathogenesis (Entrez database).

RTDR1. RTDR1 is also known as rhabdoid tumor deletion region gene 1 or ENSG00000100218 (alias MGC16968). This gene encodes a protein with no known function but with slight similarity to a yeast vacuolar protein. The gene is located in a region deleted in pediatric rhabdoid tumors of the brain, kidney and soft tissues, but mutations in this gene have not been associated with the disease (Entrez database).

Solute carrier family 35 member E4. Solute carrier family 35 member E4 is also known as SLC35E4.

Glycoprotein Ib (platelet) beta polypeptide. Glycoprotein Ib (platelet) beta polypeptide CTA-243E7.3. CTA-243E7.3 is also known as bK243E7.C22.3.

CT026_HUMAN. CT026_HUMAN is also known as C20orf26, dJ1178H5.4; DKFZP434K156.

HIST1H3A. HIST1H3A (histone; H3/A alias H3FA). Histones are basic nuclear proteins that are responsible for the nucleosome structure of the chromosomal fiber in eukaryotes. This structure consists of approximately 146 bp of DNA wrapped around a nucleosome, an octamer composed of pairs of each of the four core histones (H2A, H2B, H3, and H4). The chromatin fiber is further compacted through the interaction of a linker histone, H1, with the DNA between the nucleosomes to form higher order chromatin structures. This gene is intronless and encodes a member of the histone H3 family. Transcripts from this gene lack polyA tails; instead, they contain a palindromic termination element. This gene is found in the large histone gene cluster on chromosome 6p22-p21.3 (Entrez).

Sorting nexin 5. Sorting nexin 5 (SNX5 alias FLJ10931) is a member of the sorting nexin family. Members of this family contain a phox (PX) domain, which is a phosphoinositide binding domain, and are involved in intracellular trafficking. This protein binds to fanconi anemia complementation group A protein, but its function is unknown. This gene results in two transcript variants encoding the same protein (Entrez).

Thioredoxin reductase 2 mitochondrial precursor. Thioredoxin reductase 2 mitochondrial precursor (EC 1.8.1.9) (TR3,TR-beta) is also known as selenoprotein Z (SelZ) alias TXNRD2, TR, TR3, SELZ, TRXR2, TR-BETA. Thioredoxin reductase (TR) is a dimeric NADPH-dependent FAD containing enzyme that catalyzes the reduction of the active site disulfide of thioredoxin and other substrates. TR is a member of a family of pyridine nucleotide-disulfide oxidoreductases and is a key enzyme in the regulation of the intracellular redox environment. Three thioredoxin reductase genes have been found that encode selenocysteine containing proteins. Alternative splicing of this gene results in three distinct transcripts encoding different isoforms, one of which has been shown to be located in the mitochondria. This TR gene partially overlaps the COMT gene on chromosome 22.

RP11-554D15.1. RP11-554D15.1 is also known as bA554D15.1.

BAI1-associated protein 2-like 2. BAI1-associated protein 2-like 2 is also known as BAIAP2L2 or FLJ22582.

Smoothelin. Smoothelin (SMTN) encodes a structural protein that is found exclusively in contractile smooth muscle cells. It associates with stress fibers and constitutes part of the cytoskeleton. This gene is localized to chromosome 22q12.3, distal to the TUPLE1 locus and outside the DiGeorge syndrome deletion. Alternative splicing of this gene results in three transcript variants (Entrez database).

Cadherin-4 precursor. Cadherin-4 precursor (CDH4, CAD4, RCAD, FLJ22202, FLJ40547, MGC126700) is also known as cadherin 4 type 1, Retinal-cadherin or R-cadherin (R-CAD). This gene is a classical cadherin from the cadherin superfamily. The encoded protein is a calcium-dependent cell-cell adhesion glycoprotein comprised of five extracellular cadherin repeats, a transmembrane region and a highly conserved cytoplasmic tail. Based on studies in chicken and mouse, this cadherin is thought to play an important role during brain segmentation and neuronal outgrowth. In addition, a role in kidney and muscle development is indicated. Of particular interest are studies showing stable cis-heterodimers of cadherins 2 and 4 in cotransfected cell lines. Previously thought to interact in an exclusively homophilic manner, this is the first evidence of cadherin heterodimerization (Entrez database).

Forkhead box protein O3A. Forkhead box protein O3A (FOXO3A, AF6q21, FKHRL1, FKHRL1P2, MGC12739, MGC31925, DKFZp781A0677) belongs to the forkhead family of transcription factors which are characterized by a distinct forkhead domain. This gene likely functions as a trigger for apoptosis through expression of genes necessary for cell death. Translocation of this gene with the MLL gene is associated with secondary acute leukemia. Alternatively spliced transcript variants encoding the same protein have been observed (Entrez database).

N-acetyllactosaminide beta-1,6-N-acetylglucosaminyl-transferase. The enzyme n-acetyllactosaminide beta-1,6-N-acetylglucosaminyl-transferase (EC 2.4.1.150; GCNT2, II GCNT5, II IGNT, ULG3, AIGnT, BIGnT, CIGnT, GCNT5, NAGCT1, bA421M1.1, bA360019.2) is responsible for the formation of the blood group I antigen. The i and I antigens are determined by linear and branched poly-N-acetyllactosaminoglycans, respectively. During embryonic development in human erythrocytes, the fetal i antigen is replaced by the adult I antigen as the result of the appearance of a beta-1,6-N-acetylglucosaminyltransferase, the I-branching enzyme. This gene encodes the I-branching enzyme that converts the linear form into the branched form. Defects in this gene have been associated with adult i blood group phenotype. Several transcript variants encoding different isoforms have been described (Entrez database).

Gamma-aminobutyric-acid receptor rho-1 subunit precursor. Gamma-aminobutyric-acid receptor rho-1 subunit precursor (GABRR1) is also known as GABA(A) receptor. GABA is the major inhibitory neurotransmitter in the mammalian brain where it acts at GABA receptors, which are ligand-gated chloride channels. GABRR1 is a member of the rho subunit family (Entrez database).

OTTHUMG00000030167. OTTHUMG00000030167 is also known as CTA-243E7.3 (Vega gene ID) or bK243E7.C22.3.

OTTHUMG00000030870. OTTHUMG00000030870 is also known as CTA-503F6.1 (Vega gene ID) or bK503F6.C22.1.

OTTHUMG00000030991. OTTHUMG00000030991 is also known as LL22NC03-75B3.6 (Vega gene ID), dJ671O14.C22.6 or KIAA1644.

Pantetheinase precursor. Pantetheinase precursor (EC 3.5.1.-; VNN1 alias Tiff66; MGC116930; MGC116931; MGC116932; MGC116933) is a member of the Vanin family of proteins which share extensive sequence similarity with each other, and also with biotinidase. The family includes secreted and membrane-associated proteins, a few of which have been reported to participate in hematopoietic cell trafficking. No biotinidase activity has been demonstrated for any of the vanin proteins, however, they possess pantetheinase activity, which may play a role in oxidative-stress response. This protein, like its mouse homolog, is likely a GPI-anchored cell surface molecule. The mouse protein is expressed by the perivascular thymic stromal cells and regulates migration of T-cell progenitors to the thymus. This gene lies in close proximity to, and in same transcriptional orientation as two other vanin genes on chromosome 6q23-q24 (Entrez database).

Mitochondrial glutamate carrier 2. Mitochondrial glutamate carrier 2 is also known as Glutamate/H(+) symporter 2 (GC2) or Solute carrier family 25 member 18 (SLC25A18).

OTTHUMG00000015693. OTTHUMG00000015693 is also known as RP11-12A2.3 (Vega_gene ID) or bA12A2.3.

RP5-899B16.1. RP5-899B16.1 is also known as dJ899B16.1.

Nuclear receptor coactivator 7. Nuclear receptor coactivator 7 (NCOA7 alias ESNA1; ERAP140; MGC88425; Nbla00052; Nbla10993; dJ187J11.3).

Protein phosphatase 1 regulatory inhibitor subunit 16B. Protein phosphatase 1 regulatory inhibitor subunit 16B (PPP1R16B, KIAA0823) is also known as TGF-beta-inhibited membrane-associated protein (TIMAP, hTIMAP), CAAX box protein TIMAP or Ankyrin repeat domain protein 4 (ANKRD4). The protein encoded by this gene is membrane-associated and contains five ankyrin repeats, a protein phosphatase-1-interacting domain, and a carboxy-terminal CAAX box domain.

Synthesis of the encoded protein is inhibited by transforming growth factor beta-1. The protein may bind to the membrane through its CAAX box domain and may act as a signaling molecule through interaction with protein phosphatase-1.

Zinc finger protein SNAI1. Zinc finger protein SNAI1 is also known as Snail protein homolog or Sna protein (SNAI1 alias SNA; SNAH; SLUGH2; dJ710H13.1). The Drosophila embryonic protein snail is a zinc finger transcriptional repressor which downregulates the expression of ectodermal genes within the mesoderm. The nuclear protein encoded by this gene is structurally similar to the Drosophila snail protein, and is also thought to be critical for mesoderm formation in the developing embryo. At least two variants of a similar processed pseudogene have been found on chromosome 2 (Entrez database).

RP11-216C10.1. RP11-216C10.1 is a novel transcript.

XXbac-B444P24.7. XXbac-B444P24.7 also known as Em:AC006547.C22.7 is a novel transcript.

Reticulon 4 receptor precursor. Reticulon 4 receptor precursor is also known as Nogo receptor or Nogo-66 receptor (RTN4R alias NGR; NgR; NOGOR). This gene encodes the receptor for reticulon 4, oligodendrocyte myelin glycoprotein and myelin-associated glycoprotein. This receptor mediates axonal growth inhibition and may play a role in regulating axonal regeneration and plasticity in the adult central nervous system (Entrez database).

NFAT activation molecule 1 precursor. NFAT activation molecule 1 precursor is also known as Calcineurin/NFAT-activating ITAM-containing protein or NFAT activating protein with ITAM motif 1 (NFAM1 alias CNAIP; FLJ40652). The protein encoded by this gene is a type I membrane receptor that activates cytokine gene promoters such as the IL-13 and TNF-alpha promoters. The encoded protein contains an immunoreceptor tyrosine-based activation motif (ITAM) and is thought to regulate the signaling and development of B-cells (Entrez database).

RNA-binding protein Raly. RNA-binding protein Raly (hnRNP associated with lethal yellow homolog) D (RALY alias P542; MGC117312). In infectious mononucleosis, anti-EBNA-1 antibodies are produced which cross-react with multiple normal human proteins. The cross-reactivity is due to anti-gly/ala antibodies that cross-react with host proteins containing configurations like those in the EBNA-1 repeat. One such antigen is RALY which is a member of the heterogeneous nuclear ribonucleoprotein gene family (Entrez database).

Receptor-type tyrosine-protein phosphatase T precursor. Receptor-type tyrosine-protein phosphatase T precursor (EC 3.1.3.48; R-PTP-T alias RPTP-rho; PTPRT; RPTPrho; KIAA0283) also known as protein tyrosine phosphatase receptor type T is a member of the protein tyrosine phosphatase (PTP) family. PTPs are known to be signaling molecules that regulate a variety of cellular processes including cell growth, differentiation, mitotic cycle, and oncogenic transformation. This PTP possesses an extracellular region, a single transmembrane region, and two tandem intracellular catalytic domains, and thus represents a receptor-type PTP. The extracellular region contains a meprin-A5 antigen-PTP (MAM) domain, Ig-like and fibronectin type III-like repeats. The protein domain structure and the expression pattern of the mouse counterpart of this PTP suggest its roles in both signal transduction and cellular adhesion in the central nervous system. Two alternatively spliced transcript variants of this gene, which encode distinct proteins, have been reported (Entrez database).

RP11-191L9.1. RP11-191L9.1 is also known as bA191L9.C22.1.

RP11-410N8.3. RP11-410N8.3 is also known as bA410N8.3.

Lactosylceramide 4-alpha-galactosyltransferase. Lactosylceramide 4-alpha-galactosyltransferase (EC 2.4.1.228; A4GALT alias P1; PK; A14GALT; A4GALT1) catalyzes the transfer of galactose to lactosylceramide to form globotriaosylceramide, which has been identified as the P(k) antigen of the P blood group system. The encoded protein, which is a type II membrane protein found in the Golgi, is also required for the synthesis of the bacterial verotoxins receptor (Entrez database).

Histone H2A. Histone H2A (H1ST1H2AA alias H2AFR; bA317E16.2). Histones are basic nuclear proteins that are responsible for the nucleosome structure of the chromosomal fiber in eukaryotes. Nucleosomes consist of approximately 146 bp of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures. This gene is intronless and encodes a member of the histone H2A family. Transcripts from this gene contain a palindromic termination element (Entrez database).

Phosphatase and actin regulator 2. Phosphatase and actin regulator 2 is also known as PHACTR2 alias C6orf56; KIAA0680; DKFZp686F18175.

Pannexin-2. Pannexin-2 (PANX2 alias hPANX2; MGC119432) belongs to the innexin family. Innexin family members are the structural components of gap junctions. This protein and pannexin 1 are abundantly expressed in central nerve system (CNS) and are coexpressed in various neuronal populations. Studies in Xenopus oocytes suggest that this protein alone and in combination with pannexin 1 may form cell type-specific gap junctions with distinct properties (Entrez database).

Membrane protein MLC1. Membrane protein MLC1 (MLC1 alias VL; LVM; MLC; KIAA0027) has a sofar unknown function. However, homology to other proteins suggests that it may be an integral membrane transporter. Mutations in this gene have been associated with megalencephalic leukoencephalopathy with subcortical cysts, an autosomal recessive neurological disorder. Two transcript variants encoding the same protein but differing in the 5′ UTR have been noted for this gene (Entrez database).

RP11-318C17.1. RP11-318C17.1 is also known as bA318C17.1.

Immunoglobulin lambda constant 2. Immunoglobulin lambda constant 2 (IGLC2 alias IGLC; MGC20392; MGC45681) is also known as Ig light-chain partial Ke-Oz- polypeptide C-term or immunoglobulin lambda constant region 2 (Kern-Oz- marker).

Potassium voltage-gated channel Shab-related subfamily member 1. Potassium voltage-gated channel, Shab-related subfamily, member 1 (KCNB1 alias DRK1; KV2.1; h-DRK1). Voltage-gated potassium (Kv) channels represent the most complex class of voltage-gated ion channels from both functional and structural standpoints. Their diverse functions include regulating neurotransmitter release, heart rate, insulin secretion, neuronal excitability, epithelial electrolyte transport, smooth muscle contraction, and cell volume. Four sequence-related potassium channel genes—shaker, shaw, shab, and shal—have been identified in Drosophila, and each has been shown to have human homolog(s). This gene encodes a member of the potassium channel, voltage-gated, shab-related subfamily. This member is a delayed rectifier potassium channel and its activity is modulated by some other family members (Entrez database).

Serine/threonine-protein kinase 19. Serine/threonine-protein kinase 19 (EC 2.7.1.37; STK19 alias D6S60; D6S60E; HLA-RP1; MGC117388) is also known as RP1 protein (RP1) or G11 protein (G11). This gene encodes a serine/threonine kinase which localizes predominantly to the nucleus. Its specific function is unknown; it is possible that phosphorylation of this protein is involved in transcriptional regulation. This gene localizes to the major histocompatibility complex (MHC) class III region on chromosome 6 and expresses two transcript variants.

Tubulin alpha-8 chain. Tubulin alpha-8 chain is also known as Alpha-tubulin 8 (TUBA8 alias TUBAL2).

AP000357.3. AP000357.3 is also known as Em:AP000357.C22.3 and might be a pseudogene.

High mobility group protein HMG-I/HMG-Y. High mobility group protein HMG-I/HMG-Y (HMG-I(Y)) is also known as High mobility group AT-hook 1 or High mobility group protein A1 (HMGA1 alias HMG-R; HMGIY; MGC4242; MGC4854; MGC12816). This gene encodes a non-histone protein involved in many cellular processes, including regulation of inducible gene transcription, integration of retroviruses into chromosomes, and the metastatic progression of cancer cells. The encoded protein preferentially binds to the minor groove of A+T-rich regions in double-stranded DNA. It has little secondary structure in solution but assumes distinct conformations when bound to substrates such as DNA or other proteins. The encoded protein is frequently acetylated and is found in the nucleus. At least seven transcript variants encoding two different isoforms have been found for this gene (Entrez database).

Arylsulfatase A precursor. Arylsulfatase A precursor (EC 3.1.6.8; ASA) is also known as Cerebroside-sulfatase (contains: Arylsulfatase A component B; Arylsulfatase A component C) (ARSA alias MLD). The protein encoded by this gene hydrolyzes cerebroside sulfate to cerebroside and sulfate. Defects in this gene lead to metachromatic leucodystrophy (MLD), a progressive demyelination disease which results in a variety of neurological symptoms and ultimately death (Entrez database).

RP11-146I2.2. RP11-146I2.2 is also known as bA146I2.2.

Cold shock domain protein C2. Cold shock domain protein C2 is also known as RNA-binding protein PIPPin (CSDC2_HUMAN alias PIPPIN).

Chromosome 6 open reading frame 190. Chromosome 6 open reading frame 190 is also known as C6orf190; C6orf207; FLJ40584; bA325O24.3 or bA325O24.4.

Metalloproteinase inhibitor 3 precursor. Metalloproteinase inhibitor 3 precursor is also known as Tissue inhibitor of metalloproteinases-3 or MIG-5 protein (TIMP3 alias TIMP-3; SFD; K222; K222TA2; HSMRK222). This gene belongs to the TIMP gene family. The proteins encoded by this gene family are inhibitors of the matrix metalloproteinases, a group of peptidases involved in degradation of the extracellular matrix (ECM). Expression of this gene is induced in response to mitogenic stimulation and this netrin domain-containing protein is localized to the ECM. Mutations in this gene have been associated with the autosomal dominant disorder Sorsby's fundus dystrophy (Entrez database).

Protein C22orf13. Protein C22orf13 is also known as Protein LLN4 or CV013_HUMAN.

HARS2. HARS2 is also known as DUEB; C20orf88; MGC41905; MGC119131; bA379J5.3; bA555E18.1 and is probable a D-tyrosyl-tRNA(Tyr) deacylase (EC 3.1.-.-). The protein encoded by this gene is similar in sequence to histidyl-tRNA synthetase, which hydrolyzes D-tyrosyl-tRNA(Tyr) into D-tyrosine and free tRNA(Tyr). The encoded protein is found in the cytoplasm (Entrez database).

RP4-695O20_B.9. RP4-695O20_B.9 is also known as dJ695O20B.C22.9.

Cat eye syndrome critical region protein 1 precursor. Cat eye syndrome critical region protein 1 precursor (CECR1 alias IDGFL) is member of a subfamily of the adenosine deaminase protein family. The encoded protein may act as a growth factor and have adenosine deaminase activity. It may be responsible for some of the phenotypic features associated with cat eye syndrome. Two transcript variants encoding distinct isoforms have been identified for this gene (Entrez database).

Transcription factor 19. Transcription factor 19 is also known as Transcription factor SC1 (TCF19_HUMAN or SC1).

### SUMMARY OF THE INVENTION

The present invention provides novel and efficacious methods and nucleic acids for the classification of biological samples.

The subject matter of the invention has specific utility in the fields of medicine and/or molecular biology. In a particular aspect the utility of the present invention is to provide molecular markers and methods for the analysis thereof that may be considered an alternative to traditional histological or pathological analysis. Said molecular biological markers accordingly offer an alternative to current means such as staining and microscopic analysis.

In a particular aspect, the present invention is of particular use in determining the presence or absence of specific organ, tissue or cell types in a biological sample. Wherein said sample is heterogeneous in nature, the method according to the present invention may be used for the identification of a population or subpopulation of specific organs, tissue or cell types.

In a particular aspect, the present invention has further utility in the detection and/or classification of a cell proliferative disorder, for example but not limited to cancer.

In a particular aspect, the method of the present invention has a further alternative utility in the analysis of cellular differentiation, for example in the field of tissue engineering.

In a particular aspect, the invention solves this longstanding need in the art by providing genes, genomic sequences and/or regulatory regions thereof according to Table 1 (or to one or more of those), the expression thereof being indicative of the class of biological sample.

In a particularly preferred aspect, the methylation status of CpG positions of genes, genomic sequences and/or regulatory regions thereof according to Table 1 is used in the classification of a biological sample.

According to the invention, the provided markers, in particular the genes, genomic sequences, regulatory regions, and corresponding mRNAs, cDNAs, proteins or peptides have a particular utility in the following aspects. Thereby a single marker is used either alone or in combination with other marker or markers herein provided or not.

The herein provided markers have utility (i) for the characterization of the marker corresponding tissue or cell, (ii) for the identification of marker corresponding tissue or cell, (iii) for the isolation of marker corresponding tissue or cell, (iv) for the purification of the corresponding tissue or cell, or (v) combinations thereof. Therefore known methods, so far unreported methods, or combinations thereof are useable. Said application is useful in the field of research, diagnostics as well as therapeutics.

In addition, the herein provided markers have utility for the prospective profiling, retrospective profiling, or both of donors and/or recipients in organ transplantation procedures. The correct characterization, identification, or both of the donor and/or the recipient is mandatory during organ transplantation procedures to assure the success of the intervention. The use of the markers of the invention enables the profiling of both, donor and recipient, form which prospective or retrospective observations or conclusions about the feasibility of the procedure are drawn.

In addition, the herein provided markers have utility for histological, chemical and/or immunohistochemical analysis. Accordingly, they have utility in the fields of research as well as diagnostics, in particular for histological or pathological analysis.

In addition, the herein provided markers have utility for phylogenetic profiling of species or tissues.

The ontogenetic origin or the developmental lineage is then determined by comparison of the determined profiles.

In addition, the herein provided markers have utility for quality control of a genetically modified organism, tissue, group of cells or cell.

In addition, the herein provided markers have utility for controlling side effects in in vivo gene therapy procedures wherein genetically modified organism, tissue, group of cells or cell is used.

In addition, the herein provided markers have utility for the characterization, identification, or labelling of corresponding tissue or combinations thereof. This is of particular utility in the field of tissue bank storage and proliferation. Furthermore it has utility in a prospective as well as in a retrospective manner. The provided markers allow the individualization of samples by a precise molecular method. This is mandatory in storing biological material from patients or healthy individuals. In addition, this also advantageous for isolation or purification of tissues cells.

In addition, the herein provided markers have utility for controlling cell differentiation in stem-cell research and/or therapeutics. Cells undergo many genetic and/or epigenetic changes throughout differentiation. These changes influence the physiology of the cell and their control is mandatory in any procedure involving stem-cell in research and/or therapeutics. The provided markers allow to control this changes by giving a reference of the adult (completely differentiated) and embryonic (partially differentiated) status of the cells.

CD4+ and CD8+ lymphocytes: The herein provided markers of Table 8A and Table 9A have utility for the quantification of lymphocytes, in particular in peripheral blood. The said markers enable the identification of CD4+ and CD8+ lymphocytes among other cells in blood samples. A low number of leucocytes in blood (leucopenia) may indicate bone marrow failure (for example, due to infection, tumor, fibrosis); presence of cytotoxic substance; collagen-vascular diseases (such as lupus erythematosus); disease of the liver or spleen; or exposure to radiation. A high number of leucocytes in blood (leucocytosis) may indicate infectious diseases; inflammatory disease (such as rheumatoid arthritis or allergy); leukemia; severe emotional or physical stress; tissue damage (for example, burns); or anemia.

In addition, the herein provided markers of Table 8A and Table 9A have utility for the study of CD4 and/or CD8 T-lymphocyte infiltration in other tissues healthy or diseased. Infiltration of lymphocytes in healthy or diseased tissues is an indication of several diseases such immunological malignances or even in tumor development. The said markers represent a target for the development of molecular probes that coupled to any detection method (e.g. Fluorescent dye) allow the identification of these cells in histological preparations.

In addition, the herein provided markers of Table 8A and Table 9A have utility for identification, isolation and/or purification of CD4 T-lymphocytes and/or CD8 T-lymphocytes, in particular from surrounding tissue infiltrated by the T-lymphocytes; from blood; and/or from other body fluids.

In addition, the herein provided markers of Table 8A and Table 9A have utility for the identification of an individual. Thereby at least two samples are used. One sample is obtained from an individual and another sample is a forensic sample, in particular traces of body cells, tissues or fluids, for example but not limited to, traces of blood and/or body fluids. This is of particular utility in the field of forensic medicine or of legal medicine. As constituent of blood or body fluids, CD4 T-lymphocytes and CD8 T-lymphocytes are part of the mentioned traces. The said markers have the advantage of being stable over time because they are DNA based. In addition said markers have the advantage that they enable a highly detailed and accurate characterization of samples. Through this an unambiguous identification of an individual is enabled.

In addition, the herein provided markers of Table 8A and Table 9A have utility for diagnosing the presence or absence of a disease. Thereby the number of CD4 T-lymphocytes, CD8 T-lymphocytes or both is quantified in normalized samples of healthy individuals. The determined number of CD4 T-lymphocytes, CD8 T-lymphocytes or both are then considered as indicative for healthy condition or a diseased condition with respect to an individual. Preferably, large amount of normalized samples are considered to generate reference values of CD4 T-lymphocytes, CD8 T-lymphocytes or both for a healthy condition and/or for one or more diseased conditions. The diseased condition can be any kind of diseased condition. Preferably, the diseased condition is a disease which causes a immune reaction. For example but not limited to the diseased condition is a cancer disease, a cell proliferation disease, or HIV invection. Preferably the total number of cells present in a sample is determined. The number of CD4 T-lymphocytes, CD8 T-lymphocytes or both are then normalized to the total number of cells.

### Embryonic:

The herein provided markers of Table 8B, Table 8C, Table 9B and Table 9C have utility for the study, identification and/or quantification of fetal cells or fetal DNA circulating in maternal blood and/or amniotic fluid. During pregnancy cells and DNA from the fetus are continuously brought to the maternal blood stream as well as the amniotic fluid. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S101).

In addition, the herein provided markers of Table 8B, Table 8C, Table 9B and Table 9C have utility for the study, identification and/or quantification of fetal cells or fetal DNA from amniocentesis and/or chorionic villus sampling. This is of particular utility in the field of prenatal diagnosis. Prenatal diagnosis procedures involve the study of fetal cells obtained by amniocentesis and chorionic villus biopsies.

### Skin:

The herein provided markers of Tables 8D, G, I and Tables 9D, F, H have utility for identifying individuals from traces of skin and/or adjacent tissues (such as hair, nail pieces, etc). This is of particular utility in forensic medicine and/or legal medicine. Skin or skin adjacent tissue is usually used as study material in forensic and legal medicine. The markers provided in Table 8G and 9F have a particular utility because of the following reason: Keratinocytes constitute the external layer of the skin and therefore are the first cell type to be de-attached and a high number of these cells is expected in skin traces. Variations of one marker alone or in combination with other markers herein provided or not enable the accurate assessment of identity.

In addition, the herein provided markers of Tables 8D, G, I and Tables 9D, F, H have utility for characterizing the skin, hair, nail, or adjacent tissue of an individual.

In addition, the herein provided markers of Tables 8D, G, I and Tables 9D, F, H have utility for determining the composition of the skin, hair, nail, or adjacent tissue of an individual. Said composition being dependent from the content of at least one of the three major constituting cell types of the skin (fibroblasts, keratinocytes and melanocytes).

In addition, the herein provided markers of Tables 8D, G, I and Tables 9D, F, H have utility in the field of drugs. They have particular utility for the development of drugs as well as for the treatment with drugs. The skin, hair, nail or adjacent tissue of an individual can be characterized by means of the provided markers of Tables 8D, G, I and Tables 9D, F, H. This information can then be used to develop new drugs or to access already existing drugs with regard to skin, hair, nail etc. of an individual or to subgroups of individuals. These subgroups are for example but not limited to be characterized by a disease and/or a defined type of skin or hair, etc. The efficiency of said drugs i.e. the presence or absence of the desired effect is also characterized or monitored by means of the provided markers of Tables 8D, G, I and Tables 9D, F, H.

In addition, the herein provided markers of Tables 8D, G, I and Tables 9D, F, H have utility as prognostic and/or diagnostic markers for wound healing, in particular in the field of surgery procedures wherein the skin is affected.

### Liver:

The herein provided markers of Tables 8H and Tables 9G have utility for deducing the presence of absence of an event which affects the liver. For example but not limited to it, said event is at least one select from the group comprising liver cirrhosis; liver cancer; hepatitis A; hepatitis B; hepatitis C; healthy condition, recently or longer chemical, physical or biological exposure; recently or longer exposure to a drug, or alcohol; or treatment procedures. In the case the event is adverse, said event affecting the liver leads to the death of liver cells. In the case the event is benign, said event leads to a reduction of liver cell death. The genomic DNA of dead liver cells can then be found in the body fluids in particular in the blood of a affected individual.

In addition, the herein provided markers of Tables 8H and Tables 9G have utility for deducing the sensitivity of an individual to alcohol. Alcohol consumption may change the DNA methylation status as reviewed by Poschl et al, 2004 (Poschl G, Stickel F, Wang X D, Seitz H K. Alcohol and cancer: genetic and nutritional aspects. Proc Nutr Soc. 2004 February; 63(1):65-71.).

### Heart Muscle:

The herein provided markers of Tables 8E, Table 8F and Tables 9E have utility for deducing the presence of absence of an event or condition affecting the heart. For example but not limited to it, said event or condition is at least one select from the group comprising heart failure; heart attack; athletic capacity; healthy condition; recently or longer chemical, physical or biological exposure; recently or longer exposure to a drug; or treatment procedure. In the case the event is adverse, said event or condition affecting the heart leads to death of heart cells. In the case the event is benign, said event leads to a reduction of heart cell death. The genomic DNA of dead heart cells can then be found in the body fluids in particular in the blood of an affected individual.

### Placenta:

The herein provided markers of Table 8J and Table 9I have utility for the study, monitoring, identification and/or quantification of placental cells or placental DNA circulating in maternal blood and/or amniotic fluid. In this respect, the said markers have also utility for the isolation or purification of placental cell or placental genomic DNA. Placenta constitute an extra-embryonic fetal tissue and as such, it shares many genetic characteristics with the fetal tissue. Therefore, cells from the placenta as well as DNA from placental cells can surrogate fetal cells and fetal DNA for diagnostic means. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S101). During pregnancy placenta cells are de-attached and brought to the maternal blood stream as well as the amniotic fluid.

In addition, the herein provided markers of Table 8J and Table 9I have utility for the monitoring of embryonic development or the monitoring of placental development, in particular of extra-embryonic tissue or of interaction of extra-embryonic tissue with maternal placental tissue.

In addition, the herein provided markers of Table 8J and Table 9I have utility for the study, monitoring, identification and/or quantification of placental cells in regenerative medicine, in particular in the field of tissue engineering. Corresponding methods for the study, monitoring, identification and/or quantification of placental cells are applied in particular before and after storage, before and after cell differentiation, before and after cell proliferation, before and after cell culture expansion, and before and after tissue expansion as well as before and after transplantation.

### Sperm:

The herein provided markers of Table 8L have utility for diagnosing a male infertility related disease. A major cause of male infertility is either a low amount of sperm cells (spermatozoa) in the ejaculate (oligospermia) or a complete lack of sperm cells (spermatozoa) in the ejaculate (azoospermia). Thus, methods for the quantification of sperm cells are widely useable in diagnosing male infertility.

In addition, the herein provided markers of Table 8L have utility as a tool to access the viability of the sperm cells.

In addition, the herein provided markers of Table 8L have utility for increasing the fertility of a male individual. As said above male fertility is often limited by the amount of sperm cells in the ejaculate. Thus, male fertility can be enhanced by enriching, isolating or purifying sperm cells.

In addition, the herein provided markers of Table 8L have utility for assisted fertilization procedures. Assisted fertilization procedures are for example but not limited to intracytoplasmic sperm injection (ICSI) or in vitro fertilization (IVF). All assisted fertilization procedures require the management of sperm cells prior to the procedure. Such management comprises at least the characterization, identification, quantification, enrichment, isolation, purification of sperm cells or combinations thereof.

In addition, the herein provided markers of Table 8L have utility in the fields of forensic and/or legal medicine. By use of the said markers it is possible to determine the presence or absence of sperm in a sample. Furthermore, it is possible to identify an individual by use of said markers.

### Skeletal Muscle:

The herein provided markers of Table 8F, 8K and Table 9J have utility for characterizing the efficiency of skeletal muscle cells. This utility is of particular value in the field of sports medicine.

In addition, the herein provided markers of Table 8F, 8K and Table 9J have utility for identifying fully differentiated muscle cells in cell culture. This is of particular utility in the field of tissue engineering. Muscle cells are generate in cell culture by cultivation and differentiation of muscle cell progenitor cells. Fully differentiated skeletal muscle cells can be identified by means of the provided markers of Table 8F, 8K and Table 9J.

In addition, the herein provided markers of Table 8F, 8K and Table 9J have utility for diagnosing muscle cell associated diseases, in particular disease which are characterized by a death of muscle cells like muscular distrophy. The DNA of dead muscle cells is found in body fluids such as blood or urine. This DNA can be identified by means of the herein provided markers of Table 8F, 8K and Table 9J.

### CD8 T-Lymphocytes:

The herein provided markers of Table 8A specific only for CD8 T-lymphocytes have utility for quantifying CD8 T-lymphocytes, in particular for monitoring the immune system of individuals infected with HIV. The periodically determining of the number of CD8 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection.

### CD4+ Lymphocytes:

The herein provided markers of Table 8A specific only for CD4 T-lymphocytes have utility for quantifying CD4 T-lymphocytes, in particular for monitoring the immune system of individuals infected with HIV. The periodically determining of the number of CD4 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection.

It is particularly preferred that said biological sample is classified according to at least one parameter selected from the group consisting of the cell, organ or tissue type of said biological sample or features thereof such as disease state.

To enable this analysis the invention provides a method for the analysis of biological samples for genomic methylation associated with the classification of biological samples. Said method is characterized in that at least one nucleic acid, or a fragment thereof, from the group consisting of SEQ ID NO: 413 to SEQ ID NO: 824 is/are contacted with a reagent or series of reagents capable of distinguishing between methylated and non methylated CpG dinucleotides within the genomic sequence, or sequences of interest.

It is particularly preferred that the method and nucleic acids according to the invention are utilised for at least one of histological analysis, pathological analysis, detection and/or characterization of cell proliferative disorders and monitoring of cellular or tissue differentiation.

The DNA source may be any suitable source. Preferably, the source of the DNA sample is selected from the group consisting of cells or cell lines, histological slides, biopsies, surgical samples, tissue samples, body fluids, sputum, stool, nipple aspirate, cerebrospinal fluid, ejaculate, urine, serum, plasma, whole blood, saliva, fluids from the pleural or peritoneal cavity, cerebrospinal fluid or a smear from a epithelial surface and combinations thereof.

Furthermore, said sample may be fresh or archived and can be treated by any means standard in the art, for example but not limited to fresh-frozen, paraffin-embedded or formalin-fixed sample.

Specifically, the present invention provides a method for classifying a biological sample, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 16 contiguous nucleotides of a gene or genomic sequence selected from Table 1 said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences. Preferably said target sequences are selected from Table 3, which provides particularly preferred regions of the sequences of Table 1. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the equivalent converted sequence selected from Table 1, and contiguous regions thereof corresponding to the target sequence. It is further preferred that said converted sequences are selected from Table 3, which provides particularly preferred regions of the genes according to Table 1 (converted sequences thereof provided in Table 2).

Additional embodiments provide a method for the classification of a biological sample, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a converted sequence selected from Table 1 and complements thereof, wherein the treated DNA or the fragment thereof is either amplified to produce an amplificate, or is not amplified; and determining, based on a presence or absence of, or on a property of said amplificate, the methylation state of at least one CpG dinucleotide sequence of a gene or genomic sequence selected form Table 1, or an average, or a value reflecting an average methylation state of a plurality of CpG dinucleotide sequences thereof.

Preferably, determining comprises use of at least one method selected from the group consisting of: hybridizing at least one nucleic acid molecule comprising a contiguous sequence at least 9 nucleotides in length that is complementary to, or hybridizes under moderately stringent or stringent conditions to a converted sequence selected from Table 2 and complements thereof, hybridizing at least one nucleic acid molecule, bound to a solid phase, comprising a contiguous sequence at least 9 nucleotides in length that is complementary to, or hybridizes under moderately stringent or stringent conditions to a converted sequence selected from Table 2, and complements thereof; hybridizing at least one nucleic acid molecule comprising a contiguous sequence at least 9 nucleotides in length that is complementary to, or hybridizes under moderately stringent or stringent conditions to a sequence selected from a converted sequence selected from Table 2 (SEQ ID NO: 1650 to SEQ ID NO: 4120), and complements thereof, and extending at least one such hybridized nucleic acid molecule by at least one nucleotide base; and sequencing of the amplificate.

Further embodiments provide a method for classifying a biological sample, comprising: obtaining a biological sample having subject genomic DNA; extracting the genomic DNA; contacting the genomic DNA, or a fragment thereof, comprising one or more sequences selected from the group consisting of SEQ ID NO: 413 to SEQ ID NO: 824 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences selected from the group consisting of SEQ ID NO: 413 to SEQ ID NO: 824, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NO: 413 to SEQ ID NO: 824.

### BRIEF DESCRIPTION OF THE DRAWINGS

FIG. 1.1 to 1.403 provide the results of bisulphite sequencing as carried out in Example 2. Each individual matrix represents the sequencing data for an individual amplificate. Each of the discrete blocks of the matrix represent a single sample type and are labeled ‘A’ through ‘L’, said letters representing in each case the following tissue/cell types: A: Melanocytes; B: Heart Muscle: C: Skeletal muscle; D: Liver; E: Sperm; F: Embryonic skeletal muscle; G: Embryonic liver; H: Placental; I: Fibroblast; J: Keratinocytes; K: CD8; L: CD4. The SEQ ID NO: of the genomic region of each amplificate is shown to the left of the matrices. This may be cross referenced in Table 4 to determine the amplificate and primer sequences. Each row of a matrix represents a single CpG site within the amplificate (according to the corresponding SEQ ID NO: from Table 4) and is numbered accordingly, each column represents a single pooled DNA sample. The degree of methylation is represented by the shade of each position within the column from black representing 100% methylation to light gray representing 0% methylation. White positions represented a measurement for which no data was available.

FIG. 2: Example of RT-PCR results. Studied samples are arranged in columns and genes in rows. High DNA methylation correlates with gene silencing in MYO18B, SLC22A1 and PLG. SERPINB5 expression has been previously reported as silenced by DNA methylation (Futscher et al, 2002). ACTIN B1 is a housekeeping gene and its expression in all analyzed samples confirms the feasibility of the assay. + Control is the amplification of cDNA produced from a pool of total RNAs from several tissues. − RT is a negative control, where no reverse transcriptase was added to the cDNA synthesis reaction.

### DETAILED DESCRIPTION OF THE INVENTION

### Definitions

As used herein the term “classification” shall be taken to mean the action or process of categorising an object according to pre-determined parameters. Said categorization may be performed by humans means or by means of a computer or computer implemented means. It is particularly preferred that said parameters are phenotypic parameters, accordingly it is particularly preferred that said categorization is the assignment of a biological sample to a particular phenotypic class. In one embodiment of the method said phenotypic parameter or class is selected from the group consisting cell type, organ type, tissue type and disease status.

As used herein the term “expression” shall be taken to mean the transcription and translation of a gene. The level of expression of a gene may be determined by the analysis of any factors associated with or indicative of the level of transcription and translation of a gene including but not limited to methylation analysis, loss of heterozygosity (hereinafter also referred to as LOH), RNA expression levels and protein expression levels.

Furthermore the activity of the transcribed gene may be affected by genetic variations such as but not limited genetic mutations (including but not limited to SNPs, point mutations, deletions, insertions, repeat length, rearrangements and other polymorphisms).

The term “Observed/Expected Ratio” (“O/E Ratio”) refers to the frequency of CpG dinucleotides within a particular DNA sequence, and corresponds to the [number of CpG sites/(number of C bases×number of G bases)].

The term “CpG island” refers to a contiguous region of genomic DNA that satisfies the criteria of (1) having a frequency of CpG dinucleotides corresponding to an “Observed/Expected Ratio” >0.6, and (2) having a “GC Content” >0.5. CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “methylation level” or “level of methylation” refers to the degree of 5-methylcytosine (“5-mCyt”) at one or a plurality of CpG dinucleotides within a DNA sequence wherein one or more DNA molecules are considered.

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CC^{M}GG-3′ (top strand): 3′-GGCC-5′ (bottom strand)).

The term ‘AUC’ as used herein is an abbreviation for the area under a curve. In particular it refers to the area under a Receiver Operating Characteristic (ROC) curve. The ROC curve is a plot of the true positive rate against the false positive rate for the different possible cutpoints of a diagnostic test. It shows the tradeoff between sensitivity and specificity depending on the selected cutpoint (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975). The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo and Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong and Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley and Sons, N.Y.) at Unit 2.10.

The term polypeptide as used hereunder shall be taken to encompass all peptides, proteins and/or fragments thereof.

### Overview:

The present invention provides for molecular genetic markers that have novel utility for the classification of biological samples. In particular embodiments classification is according to at least one parameter selected from the group consisting cell type, organ type, tissue type and disease status. It is particularly preferred that the method and nucleic acids according to the invention are utilized for at least one of histological analysis, pathological analysis, detection and/or characterization of cell proliferative disorders and monitoring of cellular or tissue differentiation.

In a particularly preferred embodiment the invention provides a method for the classification of biological samples, comprising the following steps:

a) determining the expression levels of one or more genes or gene sequences according to Table 1 and/or regulatory regions thereof; and
b) classifying said biological sample according to said expression status. Said expression level may be determined by any means standard in the art including but not limited to methylation analysis, loss of heterozygosity (hereinafter also referred to as LOH), RNA expression levels and protein expression levels.

Accordingly, said method may be enabled by means of any analysis of the expression of a RNA transcribed therefrom or polypeptide or protein translated from said RNA, preferably by means of mRNA expression analysis or polypeptide expression analysis. Accordingly the present invention also provides classification assays and methods, both quantitative and qualitative for detecting the expression of the genes, genomic sequences and/or regulatory regions according to Table 1 and providing therefrom a classification of said biological sample.

Expression of mRNA transcribed from the genes or genomic regions according to Table 1, are associated with specific organ and cell types.

To detect the presence of mRNA encoding a gene or genomic sequence, a sample is obtained from a patient. The sample may be any suitable sample comprising cellular matter of the tumor, most preferably the primary tumor. Suitable sample types include cells or cell lines, histological slides, biopsies, paraffin-embedded tissue, bodily fluids, sputum, stool, nipple aspirate, cerebrospinal fluid, ejaculate, urine, blood or any other suitable biological sample and all possible combinations thereof.

The sample may be treated to extract the RNA contained therein. The resulting nucleic acid from the sample is then analyzed. Many techniques are known in the state of the art for determining absolute and relative levels of gene expression, commonly used techniques suitable for use in the present invention include in situ hybridization (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerisation chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligo dT primer and/or random hexamer primers. The cDNA thus produced is then amplified by means of PCR. (Belyavsky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridization probes (e.g. TaqMan, Lightcycler, Molecular Beacons and Scorpion) or SYBR green. The detected signal from the probes or SYBR green is then quantified either by reference to a standard curve or by comparing the Ct values to that of a calibration standard. Analysis of housekeeping genes is often used to normalize the results.

In Northern blot analysis total or poly(A)+ mRNA is run on a denaturing agarose gel and detected by hybridization to a labeled probe in the dried gel itself or on a membrane. The resulting signal is proportional to the amount of target RNA in the RNA population.

Comparing the signals from two or more cell populations or tissues reveals relative differences in gene expression levels. Absolute quantification can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labeled (e.g. radioactive labels, mass labels or fluorescent labels). The size of the target RNA, not the probe, will determine the size of the detected band, so methods such as random-primed labeling, which generates probes of variable lengths, are suitable for probe synthesis. The specific activity of the probe will determine the level of sensitivity, so it is preferred that probes with high specific activities, are used.

In an RNase protection assay, the RNA target and an RNA probe of a defined length are hybridized in solution. Following hybridization, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantification of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al. 2003), particularly preferred are probes with high specific activities.

Particularly preferred is the use of microarrays. The microarray analysis process can be divided into two main parts. First is the immobilization of known gene sequences onto glass slides or other solid support followed by hybridization of the fluorescently labeled cDNA (comprising the sequences to be interrogated) to the known genes immobilized on the glass slide. After hybridization, arrays are scanned using a fluorescent microarray scanner. Analyzing the relative fluorescent intensity of different genes provides a measure of the differences in gene expression.

DNA arrays can be generated by immobilizing presynthesized oligomers onto prepared glass slides. In this case, representative gene sequences are manufactured and prepared using standard oligomer synthesis and purification methods. These synthesized gene sequences are complementary to the genes of interest and tend to be shorter sequences in the range of 25-70 nucleotides. Alternatively, immobilized oligomers can be chemically synthesized in-situ on the surface of the slide. In situ oligomer synthesis involves the consecutive addition of the appropriate nucleotides to the spots on the microarray; spots not receiving a nucleotide are protected during each stage of the process using physical or virtual masks.

In expression profiling microarray experiments, the RNA templates used are representative of the transcription profile of the cells or tissues under study. RNA is first isolated from the cell populations or tissues to be compared. Each RNA sample is then used as a template to generate fluorescently labeled cDNA via a reverse transcription reaction. Fluorescent labeling of the cDNA can be accomplished by either direct labeling or indirect labeling methods. During direct labeling, fluorescently modified nucleotides (e.g., Cy®3- or Cy®5-dCTP) are incorporated directly into the cDNA during the reverse transcription. Alternatively, indirect labeling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabeled, but may be detectable by specific binding with a ligand which is labeled, either directly or indirectly. Suitable labels and methods for labelling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labeled with Cy®3. The resulting labeled cDNA is purified to remove unincorporated nucleotides, free dye and residual RNA. Following purification, the labeled cDNA samples are hybridized to the microarray. The stringency of hybridization is determined by a number of factors during hybridization and during the washing procedure, including temperature, ionic strength, length of time and concentration of fromamide. These factors are outlined in, for example, Sambrook et al. (Molecular Cloning: A Laboratory Manual, 2nd ed., 1989). The microarray is scanned post-hybridization using a fluorescent microarray scanner. The fluorescent intensity of each spot indicates the level of expression for that gene; bright spots correspond to strongly expressed genes, while dim spots indicate weak expression.

Once the images are obtained, the raw data must be analyzed. First, the background fluorescence must be subtracted from the fluorescence of each spot. The data is then normalized to a control sequence, such as an exogenously added RNA, or a housekeeping gene panel to account for any nonspecific hybridization, array imperfections or variability in the array setup, cDNA labeling, hybridization or washing. Data normalization allows the results of multiple arrays to be compared.

The present invention further provides methods for the detection of the presence of the polypeptide encoded by said gene sequences in a sample obtained from a patient.

Levels of polypeptide expression of the polypeptides encoded by the genes and/or genomic regions according to Table 1 are associated with the classification of biological samples.

Any method known in the art for detecting polypeptides can be used. Such methods include, but are not limited to mass-spectrometry, immunodiffusion, immunoelectrophoresis, immunochemical methods, binder-ligand assays, immunohistochemical techniques, agglutination and complement assays (e.g., see Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

Certain embodiments of the present invention comprise the use of antibodies specific to the polypeptide encoded by the genes and/or genomic regions according to Table 1.

Such antibodies are useful for the classification of biological samples. In certain embodiments production of monoclonal or polyclonal antibodies can be induced by the use of the coded polypeptide as an antigene. Such antibodies may in turn be used to detect expressed polypeptides as classification markers for biological samples. The levels of such polypeptides present may be quantified by conventional methods. Antibody-polypeptide binding may be detected and quantified by a variety of means known in the art, such as labelling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabeled, for example as used in agglutination tests, or labeled for use a wide variety of assay methods. Labels that can be used include radionuclides, enzymes, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spacial separation achieved by electrophoresis. The membrane is then incubated with a generic protein (e.g. milk protein) to bind remaining sticky places on the membrane. An antibody specific to the protein of interest is then added, said antibody being detectably labeled for example by dyes or enzymatic means (e.g. alkaline phosphatase or horseradish peroxidase). The location of the antibody on the membrane is then detected.

In an alternative embodiment of the method the proteins may be detected by means of immunochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to colored deposits at the antibody-antigen binding sites. There is a wide range of suitable sample types, antigen-antibody affinity, antibody types, and detection enhancement methods. Thus optimal conditions for immunochemical detection must be determined by the person skilled in the art for each individual case.

One approach for preparing antibodies to a polypeptide is the selection and preparation of an amino acid sequence of all or part of the polypeptide, chemically synthesising the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference). Methods for preparation of the polypeptides or epitopes thereof include, but are not limited to chemical synthesis, recombinant DNA techniques or isolation from biological samples.

In the final step of the method the biological sample is classified, as specified below.

Another aspect of the invention provides a kit for use in classifying a biological sample, comprising: a means for detecting polypeptides of a gene or genomic region according to Table 1. The means for detecting the polypeptides comprise preferably antibodies, antibody derivatives, or antibody fragments. The polypeptides are most preferably detected by means of Western blotting utilizing a labeled antibody. In another embodiment of the invention the kit further comprising means for obtaining a biological sample of the patient. Preferred is a kit, which further comprises a container suitable for containing the means for detecting the polypeptides in the biological sample of the patient, and most preferably further comprises instructions for use and interpretation of the kit results. In a preferred embodiment the kit for use in classifying biological samples, comprises: (a) a means for detecting polypeptides of a gene or genomic region according to Table 1; (b) a container suitable for containing the said means and the biological sample of the patient comprising the polypeptides wherein the means can form complexes with the polypeptides; (c) a means to detect the complexes of (b); and optionally (d) instructions for use and interpretation of the kit results.

The kit may also contain other components such as buffers or solutions suitable for blocking, washing or coating, packaged in a separate container.

Another aspect of the invention relates to a kit for use in classifying a biological sample, said kit comprising: a means for measuring the level of transcription of a gene or genomic region according to Table 1. In a preferred embodiment the means for measuring the level of transcription comprise oligomers or polynucleotides able to hybridize under stringent or moderately stringent conditions to the transcription products of a gene or genomic region according to Table 1. In a most preferred embodiment the level of transcription is determined by techniques selected from the group of Northern blot analysis, reverse transcriptase PCR, real-time PCR, RNAse protection, and microarray. In another embodiment of the invention the kit further comprises means for obtaining a biological sample of the patient. Preferred is a kit, which further comprises a container suitable for containing the means for measuring the level of transcription and the biological sample of the patient, and most preferably further comprises instructions for use and interpretation of the kit results.

In a preferred embodiment the kit for use in classifying biological samples comprises (a) a plurality of oligomers or polynucleotides able to hybridize under stringent or moderately stringent conditions to the transcription products of a gene or genomic region according to Table 1; (b) a container suitable for containing the oligomers or polynucleotides and a biological sample of the patient comprising the transcription products wherein the oligomers or polynucleotide can hybridize under stringent or moderately stringent conditions to the transcription products, (c) means to detect the hybridization of (b); and optionally, (d) instructions for use and interpretation of the kit results.

The kit may also contain other components such as hybridization buffer (where the oligomers are to be used as a probe) packaged in a separate container. Alternatively, where the oligomers are to be used to amplify a target region, the kit may contain, packaged in separate containers, a polymerase and a reaction buffer optimized for primer extension mediated by the polymerase, such as PCR.

Most preferably a kit according to the embodiments of the present invention is used for the determination of expression step of the methods according to other aspects of the invention.

In a particularly preferred embodiment the expression level of the genes, genomic sequences and/or regulatory regions according to Table 1 is determined by analysis of the level of methylation of said genes, genomic sequences and/or regulatory regions thereof. It is preferred that the level of methylation of said genes, genomic sequences and/or regulatory regions thereof is determined by determining the methylation status or level of at least one CpG dinucleotide thereof. It is further preferred that the level of methylation of said genes, genomic sequences and/or regulatory regions thereof is determined by determining the methylation status or level of a plurality of CpG dinucleotides thereof. It is further preferred that the methylation state of CpG positions within regions of the sequences of Table 1, as shown in Table 3 are analyzed. Said analysis comprises the following steps:

a) contacting genomic DNA obtained from the subject with at least one reagent, or series of reagents that distinguishes between methylated and non-methylated CpG dinucleotides within at least one target region of the genomic DNA, wherein said contiguous nucleotides comprise at least one CpG dinucleotide sequence; and
b) classifying the biological sample according to the methylation status of said target regions analyzed in i).

Genomic DNA may be isolated by any means standard in the art, including the use of commercially available kits. Briefly, wherein the DNA of interest is encapsulated in by a cellular membrane the biological sample must be disrupted and lyzed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. Preferably, the source of the DNA sample is selected from the group consisting of cells or cell lines, histological slides, biopsies, paraffin-embedded tissue, bodily fluids, ejaculate, urine, blood, and combinations thereof. Preferably, the source is biopsies, body fluids, ejaculate, urine, or blood. The genomic DNA sample is then treated in such a manner that cytosine bases which are unmethylated at the 5′-position are converted to uracil, thymine, or another base which is dissimilar to cytosine in terms of hybridization behavior. This will be understood as ‘treatment’ herein.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

The treated DNA is then analyzed in order to determine the methylation state of one or more target gene sequences (prior to the treatment) suitable for the classification of biological samples. It is preferred that the target region comprises, or hybridizes under stringent conditions to at least 16 contiguous nucleotides of the converted sequence of at least one gene or genomic sequence selected from the group consisting the genes and genomic sequences as listed in Table 1, and more preferably to the sub-regions thereof according to Table 3. It is preferred that at least one target region is selected from each of Tables 8A to 8L. It is further preferred that the sequences of said genes as described in the accompanying sequence listing are analyzed. The method of analysis may be selected from those known in the art, including those listed herein. Particularly preferred are MethyLight™, MSP™ and the use of blocking oligonucleotides as will be described herein. It is preferred that any oligonucleotides used in such analysis (including primers, blocking oligonucleotides and detection probes) should be reverse complementary, identical, or hybridize under stringent or highly stringent conditions to an at least 16-base-pair long segment of the base sequences of one or more converted sequences selected from Table 2 and sequences complementary thereto.

### Further Improvements

The present invention provides novel uses for the genes, genomic sequences and/or regulatory regions thereof according to Table 1. Additional embodiments (see Table 2) provide modified variants of SEQ ID NO: 413 to SEQ ID NO: 824, as well as oligonucleotides and/or PNA-oligomers for analysis of cytosine methylation patterns within the group consisting SEQ ID NO: 413 to SEQ ID NO: 824.

An objective of the invention comprises analysis of the methylation state of one or more CpG dinucleotides within at least one of the genomic sequences selected from the group consisting of SEQ ID NO: 413 to SEQ ID NO: 824 and sequences complementary thereto.

The disclosed invention provides treated nucleic acids, derived from genomic SEQ ID NO: 413 to SEQ ID NO: 824, wherein the treatment is suitable to convert at least one unmethylated cytosine base of the genomic DNA sequence to uracil or another base that is detectably dissimilar to cytosine in terms of hybridization. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a non-naturally occurring modified nucleic acid comprising a sequence of at least 16 contiguous nucleotide bases in length of a sequence selected from the converted sequences of Table 2. It is particularly preferred that said nucleic acid is a non-naturally occurring modified nucleic acid that is not identical to or complementary to the genomic sequences of Table 1 or other human genomic DNA.

It is further preferred that said sequence comprises at least one CpG, TpA or CpA dinucleotide and sequences complementary thereto. The sequences of SEQ ID NO: 1650 to SEQ ID NO: 4120 provide non-naturally occurring modified versions of the nucleic acid according to SEQ ID NO: 413 TO SEQ ID NO: 824, wherein the modification of each genomic sequence results in the synthesis of a nucleic acid having a sequence that is unique and distinct from said genomic sequence as follows. For each sense strand genomic DNA, e.g., SEQ ID NO:1, four converted versions are disclosed. A first version wherein “C” is converted to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequences of SEQ ID NO: 413 TO SEQ ID NO: 824 correspond to SEQ ID NO: 1650 TO SEQ ID NO: 2472. A third chemically converted version of each genomic sequences is provided, wherein “C” is converted to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO: 413 to SEQ ID NO: 824 correspond to SEQ ID NO: 3297 to SEQ ID NO: 4120.

It is particularly preferred that, fragments of the converted DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 16-base-pair long segment of the base sequences of one of SEQ ID NO: 1650 to SEQ ID NO: 4120 and sequences complementary thereto.

In an alternate embodiment of the method, the methylation status of preselected CpG positions within the nucleic acid sequences comprising one or more of SEQ ID NO: 413 to SEQ ID NO: 824 may be detected by use of methylation-specific primer oligonucleotides. This technique (MSP) has been described in U.S. Pat. No. 6,265,171 to Herman. The use of methylation status specific primers for the amplification of bisulfite converted DNA allows the differentiation between methylated and unmethylated nucleic acids. MSP primers pairs contain at least one primer which hybridizes to a bisulfite converted CpG dinucleotide. Therefore, the sequence of said primers comprises at least one CpG dinucleotide. MSP primers specific for non-methylated DNA contain a “T’ at the position of the C position in the CpG. Preferably, therefore, the base sequence of said primers is required to comprise a sequence having a length of at least 9 nucleotides which hybridizes to a converted nucleic acid sequence according to one of SEQ ID NO: 1650 to SEQ ID NO: 4120 and sequences complementary thereto, wherein the base sequence of said oligomers comprises at least one CpG dinucleotide.

A further preferred embodiment of the method comprises the use of blocker oligonucleotides. The use of such blocker oligonucleotides has been described by Yu et al., BioTechniques 23:714-720, 1997. Blocking probe oligonucleotides are hybridized to the bisulfite converted nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite converted nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridizes to a converted nucleic acid sequence according to one of SEQ ID NO: 1650 to SEQ ID NO: 4120 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the from of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas and Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut and Beck, Current Innovations and Future Trends, 1:147-57, 1995). The sensitivity with respect to nucleic acid analysis is approximately 100-times less than for peptides, and decreases disproportionately with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut and Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the next step of the method, the amplificates obtained are analyzed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesised are subsequently hybridized to an array or a set of oligonucleotides and/or PNA oligomers. In this context, the hybridization takes place in the following manner: the set of probes used during the hybridization is preferably composed of at least 2 oligonucleotides or PNA-oligomers; in the process, the amplificates serve as probes which hybridize to oligonucleotides or PNA oligomers previously bonded to a solid phase; the non-hybridized fragments are subsequently removed; said oligonucleotides or PNA oligomers contain at least one base sequence having a length of at least 9 nucleotides which is reverse complementary or identical to a segment of a sequence selected from SEQ ID NO: 1650 to SEQ ID NO: 4120; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO: 413 TO SEQ ID NO: 4120. It is preferred that at least one oligonucleotide is used to determine the status of at least one CpG dinucleotide of a gene selected from each of Tables 8A to 8L. Said oligonucleotides may also be present in the form of peptide nucleic acids. The non-hybridized amplificates are then removed. The hybridized amplificates are then detected. In this context, it is preferred that labels attached to the amplificates are identifiable at each position of the solid phase at which an oligonucleotide sequence is located.

In a particularly preferred embodiment of the method, the genomic methylation status of the CpG positions may be ascertained by means of oligonucleotide probes that are hybridized to the bisulfite converted DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which, in preferred embodiments, is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethyLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite converted DNA, and moreover for methylation analysis within CpG dinucleotides.

A further suitable method for the use of probe oligonucleotides for the assessment of methylation by analysis of bisulfite converted nucleic acids In a further preferred, the method comprises the use of template-directed oligonucleotide extension, such as MS-SNuPE as described by Gonzalgo and Jones, Nucleic Acids Res. 25:2529-2531, 1997.

In yet a further embodiment of the method, said step comprises sequencing and subsequent sequence analysis of the amplificate (Sanger F., et al., Proc Natl Acad Sci USA 74:5463-5467, 1977).

In a preferred embodiment, the methylation analysis comprises the use of an oligonucleotide or oligomer for detecting the cytosine methylation state within genomic or treated (chemically modified) DNA, according to SEQ ID NO: 413 to SEQ ID NO: 4120. Said oligonucleotide or oligomer comprising a nucleic acid sequence having a length of at least nine (9) nucleotides which hybridizes, under moderately stringent or stringent conditions (as defined herein above), to a treated nucleic acid sequence according to SEQ ID NO: 1650 to SEQ ID NO: 4120 and/or sequences complementary thereto, or to a genomic sequence according to SEQ ID NO: 413 to SEQ ID NO: 824 and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NO: 413 to SEQ ID NO: 4120, or to the complements thereof. Particularly preferred is a nucleic acid molecule that hybridizes under moderately stringent and/or stringent hybridization conditions to all or a portion of the sequences SEQ ID NO: 1650 to SEQ ID NO: 4120 but is not identical to or complementary to the equivalent genomic DNA selected from SEQ ID NO: 413 to SEQ ID NO: 4120 or other human genomic DNA. The hybridizing portion of the hybridizing nucleic acids is typically at least 9, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO: 413 to SEQ ID NO: 4120, or to the complements thereof.

Hybridizing nucleic acids of the type described herein can be used, for example, as a primer (e.g., a PCR primer), or a probe. Preferably, hybridization of the oligonucleotide probe to a nucleic acid sample is performed under stringent conditions and the probe is 100% identical to the target sequence.

Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO: 413 to SEQ ID NO: 4120 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO:1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));
where n=1, 2, 3, . . . (Y−(X−1));
where Y equals the length (nucleotides or base pairs) of SEQ ID NO: 1 (444);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=444−19=425 for either sense or antisense sets of SEQ ID NO:1, where X=20.

Preferably, the set is limited to those oligomers that comprise at least one CpG, TpG or CpA dinucleotide.

Examples of inventive 20-mer oligonucleotides include the following set of oligomers (and the antisense set complementary thereto), indicated by polynucleotide positions with reference to SEQ ID NO:1:
1-20, 2-21, 3-22, 4-23, 5-24, . . . 425-444.

Preferably, the set is limited to those oligomers that comprise at least one CpG, TpG or CpA dinucleotide.

Likewise, examples of inventive 25-mer oligonucleotides include the following set of oligomers (and the antisense set complementary thereto), indicated by polynucleotide positions with reference to SEQ ID NO:1:
1-25, 2-26, 3-27, 4-28, 5-29, . . . 420-444.

Preferably, the set is limited to those oligomers that comprise at least one CpG, TpG or CpA dinucleotide.

The present invention encompasses, for each of SEQ ID NO: 413 TO SEQ ID NO: 4120 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO: 413 to SEQ ID NO: 824. Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the from of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of at least one of the CpG dinucleotides of genomic sequences SEQ ID NO: 413 to SEQ ID NO: 824 and sequences complementary thereto, or to the corresponding CpG, TpG or CpA dinucleotide within a sequence of the treated nucleic acids according to SEQ ID NO: 1650 to SEQ ID NO: 4120 and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in treated genomic DNA (SEQ ID NO: 1650 to SEQ ID NO: 4120), or in genomic DNA (SEQ ID NO: 413 to SEQ ID NO: 824) and sequences complementary thereto. These probes enable the classification of biological samples. The set of oligomers may also be used for detecting single nucleotide polymorphisms (SNPs) in treated genomic DNA (SEQ ID NO: 1650 to SEQ ID NO: 4120), or in genomic DNA (SEQ ID NO: 413 to SEQ ID NO: 824 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO: 413 to SEQ ID NO: 4120 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the from of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the from of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilised for at least one of: in determining the presence or absence of specific organ, tissue or cell types, the detection and/or classification of a cell proliferative disorder and/or analysis of cellular differentiation.

In one embodiment of the method, this is achieved by analysis of the methylation status of at least one target sequence comprising, or hybridizing under stringent conditions to at least 16 contiguous nucleotides of a gene or sequence selected from the group consisting the genes and sequences according to Table 1 and complements thereof.

In a particularly preferred embodiment of the invention, the expression at least one of the genes, genomic sequences and/or regulatory regions thereof from each of Tables 8A to 8L is used in the classification of said sample. It is particularly preferred that said biological sample is classified according to at least one parameter selected from the group consisting of the cell, organ or tissue type of said biological sample or features thereof such as disease state.

Accordingly, in a particularly preferred embodiment the invention provides a method for the classification of biological samples, comprising the following steps:

a) determining the expression levels of one or more genes or gene sequences of each of Tables 8A to 8L and/or regulatory regions thereof; and
b) classifying said biological sample according to said expression status.

In a the most preferred embodiment thereof, the present invention provides a method for classifying a biological sample, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 16 contiguous nucleotides of at least one gene or genomic sequence selected from each of Tables 8A to 8L said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences.

Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the equivalent converted sequence selected from Tables 8A to 8L, and contiguous regions thereof corresponding to the target sequence.

In the final step of the method the classification of the biological sample according to the measured expression is determined. Table 6 and 7 provide information for the correlation of measured methylation with cell, tissue and/or organ types. The person skilled in the art will be able to interpret RNA and/or protein expression on this basis. It is generally appreciated that there is an inverse correlation between methylation and mRNA and/or polypeptide expression.

A person with ordinary skills in the art will be able to utilize the information provided in Tables 6 to 9 in order to select suitable markers according to his specific requirements. The information provided in said tables enables the selection of one or a plurality of genes or genomic sequences in order to enable the classification of biological samples.

The information provided in Tables 6 to 9 will also enable a person with ordinary skills in the art to combine suitable markers in order to classify or identify of biological samples. This can be done for example, but not limited to, by combing at least two genes or genomics regions which have a complementary and/or overlapping expression pattern. In one embodiment of the invention the information provided by Table 6 and 7 is used to for the classification of one or more biological samples. In a particularly preferred embodiment the information of Table 6 is used for the classification of a biological sample according to cell, tissue and/or organ type. In an alternative embodiment the information according to Table 7 is used for the conformation and/or monitoring of cell, tissue and/or organ type which were derived by means of cell culturing or tissue engineering processes.

### Kits

The described invention further provides a composition of matter useful for the classification of biological samples. Said composition comprising at least one nucleic acid 18 base pairs in length of a segment of a nucleic acid sequence selected from the group consisting SEQ ID NO: 1650 TO SEQ ID NO: 4120, and one or more substances taken from the group comprising: magnesium chloride, dNTP, Taq polymerase, bovine serum albumen, an oligomer in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a pretreated genomic DNA according to one of SEQ ID NO: 1650 TO SEQ ID NO: 4120 and sequences complementary thereto. It is preferred that said composition of matter comprises a buffer solution appropriate for the stabilization of said nucleic acid in an aqueous solution and enabling polymerase based reactions within said solution. Suitable buffers are known in the art and commercially available.

### Utility

The subject matter of the invention has specific utility in the fields of medicine and/or molecular biology. In particular aspects, the subject matter are one or more markers or comprises at least parts of one or more markers. Thereby a marker is a gene, a genomic sequence, a regulatory region of a gene according to Table 1 or its mRNA, cDNA or polypeptide (protein, peptide). herein also referred as molecular biological marker. According to the invention, the provided markers have novel utility for the classification of biological samples.

A utility of the present invention is to provide molecular markers and methods for the analysis thereof that may be considered an alternative to traditional histological or pathological analysis. Said molecular biological markers accordingly offer an alternative to current means such as staining and microscopic analysis.

The present invention, in particular said markers are of use in determining the presence or absence of specific organ, tissue or cell types in a biological sample. Wherein said sample is heterogeneous in nature, the method according to the present invention may be used for the identification of a population or subpopulation of specific organs, tissue or cell types. One application of this is for the determination of the tumor content of a biopsy sample which may heterogeneously comprise both tumor and normal tissue. The determination of the relative tumor content of sample is of particular interest when quantifying the presence of molecular markers by reference to the tumor content of the sample, as for example described in EP 05090318 (which is hereby incorporated by reference in its entirety).

Another application of the determination of the presence or absence of specific organ, tissue or cell types in a biological sample by means of the present invention, in particular said markers is the identification of the tissue origin of tumors or metastasis of unknown tissue origin (cancer of unknown primary, CUP). The treatment of tumors of unknown identity is a well known problem. 3-5% of all cancer diseases are cancer of unknown primary. The course of disease is characterized in that only metastasis of unknown primary tumor are detected. A tumor-specific treatment is therefore not possible. The prognosis for the affected individual is bad and the survival rate is correspondingly low. So far mainly expression-based methods for the identification of a tumor's origin are known. These methods are highly error-associated and difficult because RNA degrades quickly and easily. Therefore an exact determination of the RNA is often not possible. In addition, also a correspondent reference has to be included. But according to the present invention, the origin of tumor cells is easily determinable. Thereby genomic DNA is isolated from a fresh sample or from an archived sample. For example but not limited to, the archived sample is a formalin-fixed and/or paraffin-embedded sample. The isolated DNA is then subjected to a bisulfite conversion. Suitable methods for DNA isolation and bisulfite conversion are known in the art, for example, but not limited to, see WO 06/039563. Subsequently the converted DNA is subjected to a real time PCR based detection assays specific for the said markers. Suitable methods for real time PCR based assays are known in the art, for example but not limited to it, such an assay is a QM assay, HM assay or a MSP assay. The origin of the tumor is the identified when a tissue marker is identified which does not belong to the tissue from whom the sample was taken. Alternatively, the isolated DNA is subjected to non-real time PCR amplifications which are specific for said markers. The resulting amplicons are then detected. Suitable method for detection are known in the art. For example but not limited to it, gel electrophoresis, fluorescence, or detection by means of hybridization, for example but not limited to it, to an array. In addition, it is also possible to detect the origin of a tumor only by analyzing the genomic DNA derived from blood. Because tumors are characterized by a high rate of cell death, in particular after chemotherapy, the amount of tumor derived genomic DNA within the blood is increased in comparison to normal levels of tissue derived DNA. Therefore it is sufficient to determine if the DNA specific for a tissue is increased in the blood of a patient with cancer of unknown primary. The tissue which shows an increased level of DNA in the blood is the tissue of origin of the tumor. For this application, the said markers of the invention are used essentially according to WO 03/074730. According to it, a body fluid sample is obtained from an individual, the amount or presence of free floating DNA originating from a tissue or organ is determinated, and the presence or absence of a medical condition is determinated based on the amount or presence of the free floating DNA originating from a tissue or organ. In any case, after identification of the tumors or metastasis origin, the treatment can be adjusted. The identification also enables that the tumor can be identified in its primary tissue, from where it can then be removed. The use of the present invention, in particular said markers has several advantages in comparison to the methods for tissue identification based on expression, in particular RNA based detection: (a) DNA and DNA methylation are much more stable then RNA; (b) DNA is upstream of the regulatory cascade. This means that DNA methylation influences a lot of RNA expression. Therefore usually the analysis of a complete panel of different RNAs is necessary, while only one DNA methylation pattern in many times sufficient.

The present invention, in particular said markers have further utility in the detection and/or classification of a cell proliferative disorder, for example but not limited to cancer. It is known in the art that increased levels of circulating cells or cellular matter are a characteristic of cell proliferative disorders. The methods or markers according to the present invention enable the detection and identification of atypical levels of cells or cellular matter derived from specific organ, tissue or cell types and thereby enable the determination of the primary location of said proliferative disorder. They also enable the detection and identification of atypical levels of expression which is a sign of dedifferentiation and breakdown of the cellular regulation mechanisms. Furthermore wherein the presence of a proliferative disorder has already been detected the primary location thereof may be determined according to the methods of the present invention.

The methods or markers of the present invention have a further alternative utility in the analysis of cellular differentiation, for example in the field of tissue engineering. The molecular characterization of a biological sample as opposed to traditional histological analysis enables the improved monitoring of differentiating cell or tissue cultures. The invention solves this longstanding need in the art by providing markers i.e. genes, genomic sequences and/or regulatory regions thereof according to Table 1 (or to one or more of those), the expression thereof at the mRNA, cDNA, protein or peptide level being indicative of the class of said biological sample.

In particular, the genes, genomic sequences and/or regulatory regions thereof according to Table 8A are of use in the differentiation and/or detection of T-lymphocytes. In particular the genes, genomic sequences and/or regulatory regions thereof according to Table 8B are of use in the differentiation and/or detection of embryonic liver. In particular the genes, genomic sequences and/or regulatory regions thereof according to Table 8C are of use in the differentiation and/or detection of embryonic skeletal muscle. In particular the genes, genomic sequences and/or regulatory regions thereof according to Table 8D are of use in the differentiation and/or detection of fibroblasts. In particular the genes, genomic sequences and/or regulatory regions thereof according to Table 8E are of use in the differentiation and/or detection of heart muscle. In particular the genes, genomic sequences and/or regulatory regions thereof according to Table 8F are of use in the differentiation of heart muscle from skeletal muscle. In particular the genes, genomic sequences and/or regulatory regions thereof according to Table 8G are of use in the differentiation and/or detection of keratinocytes. In particular the genes, genomic sequences and/or regulatory regions thereof according to Table 8H are of use in the differentiation and/or detection of liver. In particular the genes, genomic sequences and/or regulatory regions thereof according to Table 8I are of use in the differentiation and/or detection of melanocytes. In particular the genes, genomic sequences and/or regulatory regions thereof according to Table 8J are of use in the differentiation and/or detection of placenta. In particular the genes, genomic sequences and/or regulatory regions thereof according to Table 8K are of use in the differentiation and/or detection of skeletal muscle. In particular the genes, genomic sequences and/or regulatory regions thereof according to Table 8L are of use in the differentiation and/or detection of sperm.

The genomic sequences (and corresponding genes) according to Table 9 are of particular use in the fields of cell culture and tissue engineering, for the confirmation and/or monitoring of particular cell, tissue and/organ types. In particular the genomic sequences according to Table 9A are suitable for the confirmation and/or monitoring of T-lymphocytes. In particular the genomic sequences according to Table 9B are suitable for the confirmation and/or monitoring of embryonic liver. In particular the genomic sequences according to Table 9C are suitable for the confirmation and/or monitoring of embryonic skeletal muscle. In particular the genomic sequences according to Table 9D are suitable for the confirmation and/or monitoring of fibroblasts. In particular the genomic sequences according to Table 9E are suitable for confirmation and/or monitoring of heart muscle. In particular the genomic sequences according to Table 9F are suitable for the confirmation and/or monitoring of keratinocytes. In particular the genomic sequences according to Table 9G are suitable for the confirmation and/or monitoring of liver. In particular the genomic sequences according to Table 9H are suitable for the confirmation and/or monitoring of melanocytes. In particular the genomic sequences according to Table 9I are suitable for the confirmation and/or monitoring of placenta. In particular the genomic sequences according to Table 9J are suitable for the confirmation and/or monitoring of skeletal muscle.

According to a particularly preferred embodiment of the invention, the methylation status of CpG positions of genes, genomic sequences and/or regulatory regions thereof according to Table 1 is used in the classification of said sample. It is particularly preferred that said biological sample is classified according to at least one parameter selected from the group consisting of the cell, organ or tissue type of said biological sample or features thereof such as disease state. According to a particular preferred embodiment, the subject matter of the invention, in particular the methods, compositions, kits, or markers are utilised for at least one of histological analysis, pathological analysis, detection and/or characterization of cell proliferative disorders and monitoring of cellular or tissue differentiation.

According to the invention, the provided markers, in particular the genes, genomic sequences, regulatory regions, and corresponding mRNAs, cDNAs, proteins or peptides have a particular utility in the following aspects. Thereby a single marker is used either alone or in combination with other marker or markers herein provided or not.

The herein provided markers have utility (i) for the characterization of the marker corresponding tissue or cell, (ii) for the identification of marker corresponding tissue or cell, (iii) for the isolation of marker corresponding tissue or cell, (iv) for the purification of the corresponding tissue or cell, or (v) combinations thereof. Therefore known methods, so far unreported methods, or combinations thereof are useable. Said application is useful in the field of research, diagnostics as well as therapeutics.

In addition, the herein provided markers have utility for the prospective profiling, retrospective profiling, or both of donors and/or recipients in organ transplantation procedures. The correct characterization, identification, or both of the donor and/or the recipient is mandatory during organ transplantation procedures to assure the success of the intervention. The use of the markers of the invention enables the profiling of both, donor and recipient, form which prospective or retrospective observations or conclusions about the feasibility of the procedure are drawn.

In addition, the herein provided markers have utility for histological, chemical and/or immunohistochemical analysis. Accordingly, they have utility in the fields of research as well as diagnostics, in particular for histological or pathological analysis.

In addition, the herein provided markers have utility for phylogenetic profiling of species or tissues. The ontogenetic origin or the developmental lineage is then determined by comparison of the determined profiles.

In addition, the herein provided markers have utility for quality control of a genetically modified organism, tissue, group of cells or cell.

In addition, the herein provided markers have utility for controlling side effects in in vivo gene therapy procedures wherein genetically modified organism, tissue, group of cells or cell is used.

In addition, the herein provided markers have utility for the characterization, identification, or labelling of corresponding tissue or combinations thereof. This is of particular utility in the field of tissue bank storage and proliferation. Furthermore it has utility in a prospective as well as in a retrospective manner. The provided markers allow the individualization of samples by a precise molecular method. This is mandatory in storing biological material from patients or healthy individuals. In addition, this also advantageous for isolation or purification of tissues cells.

In addition, the herein provided markers have utility for controlling cell differentiation in stem-cell research and/or therapeutics. Cells undergo many genetic and/or epigenetic changes throughout differentiation. These changes influence the physiology of the cell and their control is mandatory in any procedure involving stem-cell in research and/or therapeutics. The provided markers allow to control this changes by giving a reference of the adult (completely differentiated) and embryonic (partially differentiated) status of the cells.

### CD4+ and CD8+ Lymphocytes:

The herein provided markers of Table 8A and Table 9A have utility for the quantification of lymphocytes, in particular in peripheral blood. The said markers enable the identification of CD4+ and CD8+ lymphocytes among other cells in blood samples. A low number of leucocytes in blood (leucopenia) may indicate bone marrow failure (for example, due to infection, tumor, fibrosis); presence of cytotoxic substance; collagen-vascular diseases (such as lupus erythematosus); disease of the liver or spleen; or radiation. A high number of leucocytes in blood (leucocytosis) may indicate infectious diseases; inflammatory disease (such as rheumatoid arthritis or allergy); leukemia; severe emotional or physical stress; tissue damage (for example, burns); or anemia.

In addition, the herein provided markers of Table 8A and Table 9A have utility for the study of CD4 and/or CD8 T-lymphocyte infiltration in other tissues healthy or diseased. Infiltration of lymphocytes in healthy or diseased tissues is an indication of several diseases such immunological malignances or even in tumor development. The said markers represent a target for the development of molecular probes that coupled to any detection method (e.g. Fluorescent dye) allow the identification of these cells in histological preparations.

In addition, the herein provided markers of Table 8A and Table 9A have utility for identification, isolation and/or purification of CD4 T-lymphocytes and/or CD8 T-lymphocytes, in particular from surrounding tissue infiltrated by the T-lymphocytes; from blood; and/or from other body fluids.

In addition, the herein provided markers of Table 8A and Table 9A have utility for the identification of an individual. Thereby at least two samples are used. One samples is obtained from an individual and another sample is a forensic sample, in particular traces of body cells, tissues or fluids, for example but not limited to, traces of blood and/or body fluids. This is of particular utility in the field of forensic medicine or of legal medicine. As constituent of blood or body fluids, CD4 T-lymphocytes and CD8 T-lymphocytes are part of the mentioned traces. The said markers have the advantage of being stable over time because they are DNA based. In addition said markers have the advantage that they enable a highly detailed and accurate characterization of samples. Through this an unambiguous identification of an individual is enabled.

In addition, the herein provided markers of Table 8A and Table 9A have utility for diagnosing the presence or absence of a disease. Thereby the number of CD4 T-lymphocytes, CD8 T-lymphocytes or both is quantified in normalized samples of healthy individuals. The determined number of CD4 T-lymphocytes, CD8 T-lymphocytes or both are then considered as indicative for healthy condition or a diseased condition with respect to an individual. Preferably, large amount of normalized samples are considered to generate reference values of CD4 T-lymphocytes, CD8 T-lymphocytes or both for a healthy condition and/or for one or more diseased conditions. The diseased condition can be any kind of diseased condition. Preferably, the diseased condition is a disease which causes a immune reaction. For example but not limited to the diseased condition is a cancer disease, a cell proliferation disease, or HIV. Preferably the total number of cells present in a sample is determined. The number of CD4 T-lymphocytes, CD8 T-lymphocytes or both are then normalized to the total number of cells.

### Embryonic

The herein provided markers of Table 8B, Table 8C, Table 9B and Table 9C have utility for the study, identification and/or quantification of fetal cells or fetal DNA circulating in maternal blood and/or amniotic fluid. During pregnancy cells and DNA from the fetus are continuously brought to the maternal blood stream as well as the amniotic fluid. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S100).

In addition, the herein provided markers of Table 8B, Table 8C, Table 9B and Table 9C have utility for the study, identification and/or quantification of fetal cells or fetal DNA from amniocentesis and/or chorionic villus sampling. This is of particular utility in the field of prenatal diagnosis. Prenatal diagnosis procedures involve the study of fetal cells obtained by amniocentesis and chorionic villus biopsies.

### Skin

The herein provided markers of Tables 8D, G, I and Tables 9D, F, H have utility for identifying individuals from traces of skin and/or adjacent tissues (such as hair, nail pieces, etc). This is of particular utility in forensic medicine and/or legal medicine. Skin or skin adjacent tissue is usually used as study material in forensic and legal medicine. The markers provided in Table 8G and 9F have a particular utility because of the following reason. Keratinocytes constitute the external layer of the skin and therefore are the first cell type to be de-attached and a high number of these cells is expected in skin traces. Variations of one marker alone or in combination with other markers herein provided or not enable the accurate assessment of identity.

In addition, the herein provided markers of Tables 8D, G, I and Tables 9D, F, H have utility for characterizing the skin, hair, nail, or adjacent tissue of an individual.

In addition, the herein provided markers of Tables 8D, G, I and Tables 9D, F, H have utility for determining the composition of the skin, hair, nail, or adjacent tissue of an individual. Said composition being dependent from the content of at least one of the three major constituting cell types of the skin (fibroblasts, keratinocytes and melanocytes).

In addition, the herein provided markers of Tables 8D, G, I and Tables 9D, F, H have utility in the field of drugs. They have particular utility for the development of drugs as well as for the treatment with drugs. The skin, hair, nail or adjacent tissue of an individual can be characterized by means of the provided markers of Tables 8D, G, I and Tables 9D, F, H. This information can then be used to develop new drugs or to access already existing drugs with regard to skin, hair, nail etc. of an individual or to subgroups of individuals. These subgroups are for example but not limited to be characterized by a disease and/or a defined type of skin or hair, etc. The efficiency of said drugs i.e. the presence or absence of the desired effect is also characterized or monitored by means of the provided markers of Tables 8D, G, I and Tables 9D, F, H.

In addition, the herein provided markers of Tables 8D, G, I and Tables 9D, F, H have utility as prognostic and/or diagnostic markers for wound healing, in particular in the field of surgery procedures wherein the skin is affected.

### Liver

The herein provided markers of Tables 8H and Tables 9G have utility for deducing the presence of absence of an event which affects the liver. For example but not limited to it, said event is at least one select from the group comprising liver cirrhosis; liver cancer; hepatitis A; hepatitis B; hepatitis C; healthy condition, recently or longer chemical, physical or biological exposure; recently or longer exposure to a drug, or alcohol; or treatment procedures. In the case the event is adverse, said event affecting the liver leads to the death of liver cells. In the case the event is benign, said event leads to a reduction of liver cell death. The genomic DNA of dead liver cells can then be found in the body fluids in particular in the blood of a affected individual.

In addition, the herein provided markers of Tables 8H and Tables 9G have utility for deducing the sensitivity of an individual to alcohol. Alcohol consumption may change the DNA methylation status as reviewed by Poschl et al, 2004 (Poschl G, Stickel F, Wang X D, Seitz H K. Alcohol and cancer: genetic and nutritional aspects. Proc Nutr Soc. 2004 February; 63(1):65-71.).

### Heart Muscle

The herein provided markers of Tables 8E, Table 8F and Tables 9E have utility for deducing the presence of absence of an event or condition affecting the heart. For example but not limited to it, said event or condition is at least one select from the group comprising heart failure; heart attack; athletic capacity; healthy condition; recently or longer chemical, physical or biological exposure; recently or longer exposure to a drug; or treatment procedure. In the case the event is adverse, said event or condition affecting the heart leads to death of heart cells. In the case the event is benign, said event leads to a reduction of heart cell death. The genomic DNA of dead heart cells can then be found in the body fluids in particular in the blood of an affected individual.

### Placenta

The herein provided markers of Table 8J and Table 9I have utility for the study, monitoring, identification and/or quantification of placental cells or placental DNA circulating in maternal blood and/or amniotic fluid. In this respect, the said markers have also utility for the isolation or purification of placental cell or placental genomic DNA. Placenta constitute an extra-embryonic fetal tissue and as such, it shares many genetic characteristics with the fetal tissue. Therefore, cells from the placenta as well as DNA from placental cells can surrogate fetal cells and fetal DNA for diagnostic means. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S100). During pregnancy placenta cells are de-attached and brought to the maternal blood stream as well as the amniotic fluid.

In addition, the herein provided markers of Table 8J and Table 9I have utility for the monitoring of embryonic development or the monitoring of placental development, in particular of extra-embryonic tissue or of interaction of extra-embryonic tissue with maternal placental tissue.

In addition, the herein provided markers of Table 8J and Table 9I have utility for the study, monitoring, identification and/or quantification of placental cells in regenerative medicine, in particular in the field of tissue engineering. Corresponding methods for the study, monitoring, identification and/or quantification of placental cells are applied in particular before and after storage, before and after cell differentiation, before and after cell proliferation, before and after cell culture expansion, and before and after tissue expansion as well as before and after transplantation.

### Sperm

The herein provided markers of Table 8L have utility for diagnosing a male infertility related disease. A major cause of male infertility is either a low amount of sperm cells (spermatozoa) in the ejaculate (oligospermia) or a complete lack of sperm cells (spermatozoa) in the ejaculate (azoospermia). Thus, methods for the quantification of sperm cells are widely useable in diagnosing male infertility.

In addition, the herein provided markers of Table 8L have utility as a tool to access the viability of the sperm cells.

In addition, the herein provided markers of Table 8L have utility for increasing the fertility of a male individual. As said above male fertility is often limited by the amount of sperm cells in the ejaculate. Thus, male fertility can be enhanced by enriching, isolating or purifying sperm cells.

In addition, the herein provided markers of Table 8L have utility for assisted fertilization procedures. Assisted fertilization procedures are for example but not limited to intracytoplasmic sperm injection (ICSI) or in vitro fertilization (IVF). All assisted fertilization procedures require the management of sperm cells prior to the procedure. Such management comprises at least the characterization, identification, quantification, enrichment, isolation, purification of sperm cells or combinations thereof.

In addition, the herein provided markers of Table 8L have utility in the fields of forensic and/or legal medicine. By use of the said markers it is possible to determine the presence or absence of sperm in a sample. Furthermore, it is possible to identify an individual by use of said markers.

### Skeletal Muscle

The herein provided markers of Table 8F, 8K and Table 9J have utility for characterizing the efficiency of skeletal muscle cells. This utility is of particular value in the field of sports medicine.

In addition, the herein provided markers of Table 8F, 8K and Table 9J have utility for identifying fully differentiated muscle cells in cell culture. This is of particular utility in the field of tissue engineering. Muscle cells are generate in cell culture by cultivation and differentiation of muscle cell progenitor cells. Fully differentiated skeletal muscle cells can be identified by means of the provided markers of Table 8F, 8K and Table 9J.

In addition, the herein provided markers of Table 8F, 8K and Table 9J have utility for diagnosing muscle cell associated diseases, in particular disease which are characterized by a death of muscle cells like muscular distrophy. The DNA of dead muscle cells is found in body fluids such as blood or urine. This DNA can be identified by means of the herein provided markers of Table 8F, 8K and Table 9J.

### CD8 T-Lymphocytes

The herein provided markers of Table 8A specific only for CD8 T-lymphocytes have utility for quantifying CD8 T-lymphocytes, in particular for monitoring the immune system of individuals infected with HIV. The periodically determining of the number of CD8 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection.

### CD4+ Lymphocytes

The herein provided markers of Table 8A specific only for CD4 T-lymphocytes have utility for quantifying CD4 T-lymphocytes, in particular for monitoring the immune system of individuals infected with HIV. The periodically determining of the number of CD4 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection.

According to a preferred embodiment of the invention, the provided markers, in particular the genes, genomic sequences, regulatory regions, and corresponding mRNAs, cDNAs, proteins or peptides are used in the following aspects. Thereby a single marker is used either alone or in combination with other marker or markers herein provided or not.

In a preferred embodiment, at least one of the provided markers is used (i) for the characterization of the marker corresponding tissue or cell, (ii) for the identification of marker corresponding tissue or cell, (iii) for the isolation of marker corresponding tissue or cell, (iv) for the purification of the corresponding tissue or cell, or (v) combinations thereof. Therefore known methods, so far unreported methods, or combinations thereof are useable. Said application is useful in the field of research, diagnostics as well as therapeutics. As an example, but not limited to it, this application is illustrated in more detail for the marker PDGFB SEQ ID NO: 508. All the herein provided markers can be applied and used in the same way like PDGFB SEQ ID NO: 508 correspondingly to their assignment to respective tissues, organs or cells according to Tables 8 A-L or Tables 9 A-J. Of course, corresponding markers can be alternatively used. Thereby a corresponding marker of PDGFB SEQ ID NO: 508 is, for example but not limited to, genomic DNA derived from or associated with PDGFB SEQ ID NO: 508; methylation specifically converted DNA derived from PDGFB SEQ ID NO: 508; mRNA, cDNA, protein, or peptide each of which derived at least in parts from PDGFB SEQ ID NO: 508. If the case may be, a person skilled in the art knows how to adjust the presented procedure. As shown in Table 6, PDGFB SEQ ID NO: 508 is a marker for adult liver because the CpG dinucleotides of PDGFB SEQ ID NO: 508 are methylated within the range of 75-100% in liver and only within the range of 0-25% in T-lymphocytes (CD4, CD8), embryonic tissue (embryonic liver, embryonic skeletal muscle), skin (melanocytes, keratinocytes, fibroblasts), heart muscle, placenta, sperm, or skeletal muscle.

Correspondingly, for example but not limited to it, a method for characterization and/or identification of a cell or tissue type of a sample comprises the following steps:

1. Providing of a sample, the sample being derived from an individual and comprising genomic DNA. Preferably, the genomic DNA is purified by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing a cell or tissue type by determining the methylation state or the methylation level of at least one CpG position within the sequence of PDGFB SEQ ID NO: 508 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Optional, deducing the presence or absence of a cell type or tissue type within the provided sample from the presence or absence of a methylation state or methylation level for said at least one CpG.

For example but not limited to, a method for isolation and/or purification of a cell or group of cells comprises the following steps:

1. Providing of a sample, the sample being derived from an individual and comprising one or more cells.
2. Binding of at least one probe to one or more CpG positions within the sequence of PDGFB SEQ ID NO: 508 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells.
3. Isolating and/or purifying a cell or group of cells from the provided sample by means of the attached probes and their corresponding tags, respectively. A person skilled in the art is aware of suitable methods. Said methods are based on chemical, physical or biological properties of the attached probes or corresponding tags. For example but not limited to, the isolation is performed (i) by means of affinity cromatography, wherein the probe is attached to a tag that is recognized by an antibody immobilized on a column; (ii) by means of magnetic beads, wherein a magnetic bead is directly or indirectly bound to an attached probe and wherein a magnetic field is applied; or (iii) by means of fluorescent activated cell sorting, wherein the used tag is a fluorescent dye.

In a preferred embodiment, at least one of the provided markers is used for the prospective profiling, retrospective profiling, or both of donors and/or recipients in organ transplantation procedures. The correct characterization, identification, or both of the donor and/or the recipient is mandatory during organ transplantation procedures to assure the success of the intervention. The use of the markers of the invention enables the profiling of both, donor and recipient, form which prospective or retrospective observations or conclusions about the feasibility of the procedure are drawn. As an example, but not limited to it, this application is illustrated in more detail for the marker FOXC1 SEQ ID NO: 579. All other markers of Table 8E, Table 8F and Table 9F are applied and used like FOXC1 SEQ ID NO: 579 for heart muscle. The other herein provided markers are applied and used according their assignment to the Tables 8 A-L or Tables 9 A-J. Of course, corresponding markers can also be alternatively used. Thereby corresponding markers of FOXC1 SEQ ID NO: 579 are, for example but not limited to, genomic DNA derived from or associated with FOXC1 SEQ ID NO: 579; methylation specifically converted DNA derived from FOXC1 SEQ ID NO: 579; mRNA, cDNA, protein, or peptide each of which derived at least in parts from FOXC1 SEQ ID NO: 579. If the case may be, a person skilled in the art knows how to adjust the presented procedures. As shown in Table 6, FOXC1 SEQ ID NO: 579 is a marker for heart muscle because the CpG dinucleotides of FOXC1 SEQ ID NO: 579 are methylated within the range of 25-75% in heart muscle and only within the range of 0-25% in T-lymphocytes (CD4, CD8), embryonic tissue (embryonic liver, embryonic skeletal muscle), skin (melanocytes, keratinocytes, fibroblasts), liver, placenta, sperm, or skeletal muscle.

Correspondingly, for example but not limited to it, a method for the prospective profiling, retrospective profiling, or both of a donor and/or recipient in organ or tissue transplantation procedures comprises the following steps:

1. Providing of at least one sample, at least one sample being derived from a donor individual and/or at least one sample being derived from a recipient individual. Genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each of the at least one sample by determining the methylation state or the methylation level of at least one CpG position within the sequence of FOXC1 SEQ ID NO: 579 of the provided samples. Thereby a profile is generated comprising the methylation information of all characterized CpG positions of FOXC1 SEQ ID NO: 579. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine anti-bodies.
3. Comparing the determined profiles, in particular the methylation state or methylation level of the analyzed at least one CpG position of the donor sample(s) and/or the recipient sample(s). Thereby the profiling is prospective or retrospective depending from the point in time the samples were collected i.e. before or after the transplantation procedure. A person skilled in the art knows then how to interpret the profiling to give an estimate on the probability of success of the intervention.

In a preferred embodiment, at least one of the provided markers is detected in studies by histological, chemical and/or immunohistochemical means. Said embodiment is useful in the fields of research as well as diagnostics, in particular for histological or pathological analysis. According to the said embodiment, the detection occurs by one or more probes that specifically bind to an epitop, peptide, protein, cDNA, mRNA, and/or at least one methylation state or level of at least one of the provided markers according to Tables 8 A-L or Table 9 A-J. Thereby a probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative. In addition, the probe is labeled directly or indirectly with a dye, protein, enzyme, metal, bead or chemical compound suitable for detection. As an example, but not limited to it, this embodiment is illustrated in more detail for the marker CMAH SEQ ID NO: 570. All other markers of Table 8G and Table 9F are applied and used like CMAH SEQ ID NO: 570 for keratinocytes. The other herein provided markers are applied and used according their assignment to the Tables 8 A-L or Tables 9 A-J. Of course, corresponding markers can also be alternatively used. Thereby corresponding markers of CMAH SEQ ID NO: 570 are, for example but not limited to, genomic DNA derived from or associated with CMAH SEQ ID NO: 570; methylation specifically converted DNA derived from CMAH SEQ ID NO: 570; mRNA, cDNA, protein, or peptide each of which derived at least in parts from CMAH SEQ ID NO: 570. If the case may be, a person skilled in the art knows how to adjust the presented procedure. As shown in Table 6, CMAH SEQ ID NO: 570 is a marker for keratinocytes and sperm because the CpG dinucleotides of CMAH SEQ ID NO: 570 are methylated only within the range of 0-25% in keratinocytes and sperm and within the range of 75-100% in T-lymphocytes (CD4, CD8), embryonic tissue (embryonic liver, embryonic skeletal muscle), melanocytes, fibroblasts, heart muscle, liver, or skeletal muscle. Keratinocytes and sperm can easily be distinguished by their different morphological appearance and physiological occurrence.

Correspondingly, for example but not limited to it, a method for histological or pathological analysis, comprises

1. Providing of a sample, comprising genomic DNA;
2. Contacting the genomic DNA or a derivative of it with at least one probe which is specific for at least one differentially methylated CpG position of the marker CMAH SEQ ID NO: 570. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art is aware of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen).

In a preferred embodiment, at least one of the provided markers is applied to phylogenetic profiling of species or tissues. The ontogenetic origin or the developmental lineage is then determined by comparison of the determined profiles. As an example, but not limited to it, this application is illustrated in more detail for the marker AIM1 SEQ ID NO: 538. All other markers of Table 8J and Table 9I are applied and used like AIM1 SEQ ID NO: 538 for placenta. The other herein provided markers are applied and used according their assignment to the Tables 8 A-L or Tables 9 A-J. Of course, corresponding markers can also be alternatively used. Thereby corresponding markers of AIM1 SEQ ID NO: 538 are, for example but not limited to, genomic DNA derived from or associated with AIM1 SEQ ID NO: 538; methylation specifically converted DNA derived from AIM1 SEQ ID NO: 538; mRNA, cDNA, protein, or peptide each of which derived at least in parts AIM1 SEQ ID NO: 538. If the case may be, a person skilled in the art knows how to adjust the presented procedures. As shown in Table 6, AIM1 SEQ ID NO: 538 is a marker for placenta because the CpG dinucleotides of AIM1 SEQ ID NO: 538 are methylated within the range of 25-75% in placenta and only within the range of 0-25% in T-lymphocytes (CD4, CD8), embryonic tissue (embryonic liver, embryonic skeletal muscle), skin (melanocytes, keratinocytes, fibroblasts), liver, heart muscle, sperm, or skeletal muscle.

Correspondingly, for example but not limited to it, a method for phylogenetic profiling of species or tissues, comprises

1. Providing of at least one sample, each sample comprising genomic DNA. Genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each of the at least one sample by determining the methylation state or the methylation level of at least one CpG position within the sequence of AIM1 SEQ ID NO: 538 of the provided samples. Thereby a profile is generated comprising the methylation information of all characterized CpG positions of AIM1 SEQ ID NO: 538. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine anti-bodies.

The profiles of the one or more respective samples are then compared with each other or with at least one reference profile. According to methods or algorithms known to those skilled in the art, the ontogenetic origin of a cell, group of cells, tissue or organ or the developmental lineage of a cell, group of cells, tissue or organ is determined.

In a preferred embodiment, at least one of the provided markers is applied for quality control of a genetically modified organism, tissue, group of cells or cell. As an example, but not limited to it, this embodiment is illustrated in more detail for the marker TBC1D10A SEQ ID NO: 700. All other markers of Table 8E, 8F and Table 9E are applied and used like TBC1D10A SEQ ID NO: 700 for heart muscle. The other herein provided markers are applied and used according their assignment to the Tables 8 A-L or Tables 9 A-J. Of course, corresponding markers can also be alternatively used. Thereby corresponding markers of TBC1D10A SEQ ID NO: 700 are, for example but not limited to, genomic DNA derived from or associated with TBC1D10A SEQ ID NO: 700; methylation specifically converted DNA derived from TBC1D10A SEQ ID NO: 700; mRNA, cDNA, protein, or peptide each of which derived at least in parts from TBC1D10A SEQ ID NO: 700. If the case may be, a person skilled in the art knows how to adjust the presented procedures. TBC1D10A SEQ ID NO: 700 is a marker for heart muscle because a) the CpG dinucleotides of TBC1D10A SEQ ID NO: 700 are methylated to a significantly higher extend in the heart muscle and sperm than in other tissues (see Table 6); and b) heart muscle and sperm can be easily distinguished morphologically or by means of other markers. According to Table 6 75-100% of the CpG dinucleotides of the marker TBC1D10A SEQ ID NO: 700 is methylated in heart muscle and sperm; 0-25% is methylated in T-lymphocytes (CD4, CD8), embryonic tissue (embryonic liver, embryonic skeletal muscle), skin (melanocytes, keratinocytes, fibroblasts), and placenta; and 25-75% is methylated in liver and skeletal muscle.

Correspondingly, for example but not limited to it, a method for quality control of a genetically modified organism, tissue, group of cells or cell, comprises

1. Providing of at least one sample of or derived from the genetically modified organism, tissue, group of cells or cell, each sample comprising genomic DNA. The genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each of the at least one sample by determining the methylation state or the methylation level of at least one CpG position within the sequence of TBC1D10A SEQ ID NO: 700 of the provided samples. Thereby a profile is generated comprising the methylation information of all characterized CpG positions of TBC1D10A SEQ ID NO: 700. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine anti-bodies.
3. Comparing the said profiles with each other or with reference profiles. A person skilled in the art knows to interpret correspondingly said comparison and to deduce therefrom the quality of the genetically modified organism, tissue, group of cells or cell. Thereby a person skilled in the art is enabled to draw prospectively or retrospectively conclusions on the presence or absence of side effects if said genetically modified organism, tissue, group of cells or cell is brought into contact with other organisms, tissues, groups of cells or cells.

For example but not limited to, a method for quality control of an genetically modified organism, tissue, group of cells or cell, comprises

1. Providing of at least one first sample of or derived from an organism, tissue, group of cells or cell being not genetically modified and at least one second sample of or derived from a correspondent organism, tissue, group of cells or cell being genetically modified. Thereby each sample comprises genomic DNA. The genomic DNA is purified from said first and second samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each of the at least one first and second samples by determining the methylation state or the methylation level of at least one CpG position within the sequence of TBC1D10A SEQ ID NO: 700 of the provided samples. Thereby a first and second profile is generated comprising the methylation information of all characterized CpG positions of TBC1D10A SEQ ID NO: 700 of the respective samples. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine anti-bodies.
3. Comparing the said first and second profile with each other or with reference profiles.
4. Deducing the presence or absence of side effects, wherein said side effects are characterized in that changes are introduced into regions of the genome which were not target of the genetic modification. This application is of particular interest to exclude unwanted physiological alterations of the cell, group of cells, tissue or organism. The reason for this is unwanted physiological alterations are mainly caused by genetic modification of non-target genomic DNA regions.

According to a particular preferred embodiment, the at least one first sample is derived from an organism, tissue, group of cells or cell before a genetic modification and the at least one second sample is derived thereof after said genetic modification.

In a preferred embodiment, at least one of the provided markers is applied for controlling side effects in in vivo gene therapy procedures wherein genetically modified organism, tissue, group of cells or cell is used. As an example, but not limited to it, this embodiment is illustrated in more detail for the marker GPX5 SEQ ID NO: 574. All other markers of Table 8A and Table 9A are applied and used correspondingly as GPX5 SEQ ID NO: 574 for T-lymphocytes. The other herein provided markers are applied and used according their assignment to the Tables 8 A-L or Tables 9 A-J. Of course, corresponding markers can also be alternatively used. Thereby corresponding markers of GPX5 SEQ ID NO: 574 are, for example but not limited to, genomic DNA derived from or associated with GPX5 SEQ ID NO: 574; methylation specifically converted DNA derived from GPX5 SEQ ID NO: 574; mRNA, cDNA, protein, or peptide each of which derived at least in parts from GPX5 SEQ ID NO: 574. If the case may be, a person skilled in the art knows how to adjust the presented procedures. As shown in Table 6, GPX5 SEQ ID NO: 574 is a marker for T-lymphocytes because the CpG dinucleotides of GPX5 SEQ ID NO: 574 are methylated within the range of 0-25% in CD4 T-lymphocytes as well as in CD 8 T-lymphocytes and within the range of 75-100% in embryonic tissue (embryonic liver, embryonic skeletal muscle), skin (melanocytes, keratinocytes, fibroblasts), placenta, liver, heart muscle, sperm, or skeletal muscle.

For example but not limited to, a method for controlling side effects in in vivo gene therapy procedures, comprises

1. Providing of at least one untreated sample derived from an individual and at least one treated sample of said individual. Thereby the samples are derived from respective body regions and each of the samples comprises genomic DNA. The genomic DNA is purified from said first and second samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each of the at least one first and second samples by determining the methylation state or the methylation level of at least one CpG position within the sequence of GPX5 SEQ ID NO: 574 of the provided samples. Thereby a first and second profile is generated comprising the methylation information of all characterized CpG positions of GPX5 SEQ ID NO: 574 of the respective samples. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine anti-bodies.
3. Comparing the said first and second profile with each other or with reference profiles.
4. Deducing the presence or absence of side effects, wherein said side effects are characterized in that changes are introduced into regions of the genome which were not target of the genetic modification. This application is of particular interest to exclude unwanted physiological alterations for the individual. The reason for this unwanted physiological alterations are mainly caused by genetic modification of non-target genomic DNA regions.

According to a particular preferred embodiment, the at least one untreated sample is derived before the gene therapy and the at least one treated sample is derived after said gene therapy.

In a preferred embodiment, at least one of the provided markers is applied for the characterization, identification, or labelling of corresponding tissue or combinations thereof. Said embodiment is of particular use in the field of tissue bank storage and proliferation. Furthermore it can be used in a prospective as well as in a retrospective manner. The provided markers allow the individualization of samples by a precise molecular method. This is mandatory in storing biological material from patients or healthy individuals. In addition, this also advantageous for isolation or purification of tissues cells. As an example, but not limited to it, this application is illustrated in more detail for the marker TCN2 SEQ ID NO: 470. All other markers of Table 8L are applied and used like TCN2 SEQ ID NO: 470 for sperm. The other herein provided markers are applied and used according their assignment to the Tables 8 A-L or Tables 9 A-J. Of course, corresponding markers can also be alternatively used. Thereby corresponding markers of TCN2 SEQ ID NO: 470 are, for example but not limited to, genomic DNA derived from or associated with TCN2 SEQ ID NO: 470; methylation specifically converted DNA derived from TCN2 SEQ ID NO: 470; mRNA, cDNA, protein, or peptide each of which derived at least in parts from TCN2 SEQ ID NO: 470. If the case may be, a person skilled in the art knows how to adjust the presented procedures. As shown in Table 6, TCN2 SEQ ID NO: 470 is a marker for sperm because the CpG dinucleotides of TCN2 SEQ ID NO: 470 are methylated within the range of 75-100% in sperm and to only an extend of 0-25% in T-lymphocytes (CD4, CD8), embryonic tissue (embryonic liver, embryonic skeletal muscle), skin (melanocytes, keratinocytes, fibroblasts), placenta, liver, heart muscle, or skeletal muscle.

Correspondingly, for example but not limited to it, a method for characterizing a tissue or cell, comprises

1. Providing of at least one sample comprising genomic DNA. The genomic DNA is purified from said sample(s), preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each sample by determining the methylation state or the methylation level of at least one CpG position within the sequence of TCN2 SEQ ID NO: 470 of the provided sample(s). Thereby a profile is generated comprising the methylation information of all characterized CpG positions of TCN2 SEQ ID NO: 470 of the respective sample(s). A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

For example but not limited to it, a method for labelling a tissue or cell, comprises in addition to the said method for characterizing a tissue or cell:

3. Labelling said tissue or cell by means of the methylation status or level of one or more of the analyzed CpG positions of TCN2 SEQ ID NO: 470. According to a preferred embodiment the labelling is achieved by assigning at least one distinct methylation status or level of at least one analyzed CpG dinucleotide of TCN2 SEQ ID NO: 470 to said tissue or cell. Thereby the assigned methylation status or level(s) are specific for said tissue or cell.

For example but not limited to it, a method for identifying a tissue or cell, comprises in addition to the said method for characterizing a tissue or cell:

3. Identifying a tissue or cell or an individual from whom the sample is derived from by comparison of the determined TCN2 profile of said samples with a reference TCN2 profile.

For example but not limited to it, a method for profiling a tissue type or cell type, comprises in addition to the said method for characterizing a tissue or cell:

3. Comparing the determined TCN2 profiles of said samples with each other and/or with at least one reference TCN2 profile and considering the group of same methylation status or levels of correspondent CpG dinucleotides of different samples as a tissue type or cell type profile.

A person skilled in the art knows to combine the said methods for characterizing a tissue or cell; for labelling a tissue or cell; for identifying a tissue or cell; and for profiling a tissue type or cell type with state of the art methods for tissue or cell isolation or purification.

In a preferred embodiment, at least one of the provided markers is applied for controlling cell differentiation in stem-cell research and/or therapeutics. Cells undergo many genetic and/or epigenetic changes throughout differentiation. These changes influence the physiology of the cell and their control is mandatory in any procedure involving stem-cell in research and/or therapeutics. The provided markers enable to control this changes by giving a reference of the adult (completely differentiated) and embryonic (partially differentiated) status of the cells. As an example, but not limited to it, this application is illustrated in more detail for the marker RPL3 SEQ ID NO: 529. All other markers of Table 8H and Table 9G are applied and used like RPL3 SEQ ID NO: 529 for adult liver. The other herein provided markers are applied and used according their assignment to the Tables 8 A-L or Tables 9 A-J. Of course, corresponding markers can also be alternatively used. Thereby corresponding markers of RPL3 SEQ ID NO: 529 are, for example but not limited to, genomic DNA derived from or associated with RPL3 SEQ ID NO: 529; methylation specifically converted DNA derived from RPL3 SEQ ID NO: 529; mRNA, cDNA, protein, or peptide each of which derived at least in parts from RPL3 SEQ ID NO: 529. If the case may be, a person skilled in the art knows how to adjust the presented procedures. As shown in Table 6, RPL3 SEQ ID NO: 529 is a marker for sperm because the CpG dinucleotides of RPL3 SEQ ID NO: 529 are methylated within the range of 25-75% in adult liver and within the range of 75-100% in T-lymphocytes (CD4, CD8), embryonic tissue (embryonic liver, embryonic skeletal muscle), skin (melanocytes, keratinocytes, fibroblasts), placenta, sperm, heart muscle, or skeletal muscle.

Correspondingly, for example but not limited to it, a method for controlling cell differentiation in stem-cell research and/or therapeutics, comprises

1. Providing of a sample comprising genomic DNA. The genomic DNA is purified from said sample(s), preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing said sample by determining the methylation state or the methylation level of at least one CpG position within the sequence of RPL3 SEQ ID NO: 529 of the provided sample. Thereby a profile is generated comprising the methylation information of all characterized CpG positions of RPL3 SEQ ID NO: 529 of the respective sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Comparing said RPL3 profile of said sample with at least one reference RPL3 profile and deducing therefrom if a desired differentiation status of one or more cells of the sample is met or not. Through this a controlling of cell differentiation is achieved.

According to Table 6, the herein provided markers are assigned to different tissues (see Tables 8 A-L or Tables 9 A-J). In the following, applications are described which are only applicable for the named tissue(s).

### CD4+ and CD8+ Lymphocytes:

In a preferred embodiment, the herein provided markers of Table 8A and Table 9A are used for the quantification of lymphocytes, in particular in peripheral blood.

Low number of leucocytes in blood (leucopenia) may indicate:
bone marrow failure (for example, due to infection, tumor, fibrosis),
presence of cytotoxic substance,
collagen-vascular diseases (such as lupus erythematosus),
disease of the liver or spleen,
exposure to radiation,

High number of leucocytes in blood (leucocytosis) may indicate:
infectious diseases,
inflammatory disease (such as rheumatoid arthritis or allergy),
leukemia,
severe emotional or physical stress,
tissue damage (for example, burns),
anemia.

Said markers enable the identification of CD4+ and CD8+ lymphocytes among other cells in blood samples. For example but not limited to it, the differential methylation of FBLN1 SEQ ID NO: 426 is used. According to Table 6, the differential methylation of FBLN1 SEQ ID NO: 426 is marker for CD4 T-lymphocytes as well as for CD8 T-lymphocytes because the CpG dinucleotides of FBLN1 SEQ ID NO: 426 are methylated within the range of 25-75% in CD4 and CD8 T-lymphocytes while other tissues show a different extend of methylation. Of course, corresponding markers can also be alternatively used. Thereby corresponding markers of FBLN1 SEQ ID NO: 426 are, for example but not limited to, genomic DNA derived from or associated with FBLN1 SEQ ID NO: 426; methylation specifically converted DNA derived from FBLN1 SEQ ID NO: 426; mRNA, cDNA, protein, or peptide each of which derived at least in parts from FBLN1 SEQ ID NO: 426. If the case may be, a person skilled in the art knows how to adjust the presented procedures.

Correspondingly, for example but not limited to it, a method for the quantification of CD4 and/or CD8 T-lymphocytes, comprises:

1. Providing of a sample, comprising genomic DNA;
2. Contacting the genomic DNA or a derivative of it with at least one probe which is specific for at least one differentially methylated CpG position of the marker FBLN1 SEQ ID NO: 426. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art is aware of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen). A person skilled in the art knows further suitable methods for detection.
4. Quantifying the detection reaction in a manner so that the detected signal is indicative for the amount of probe or label therewith also for the amount of CD4 and/or CD8 T-lymphocytes. A person knows suitable methods for quantification.

In a particular preferred embodiment, the said method is also a method for isolation of CD4 and CD8 T-lymphocytes. Said method additional comprises the separation of CD4 and CD8 T-lymphocytes from other cells, tissue, or molecules of a sample by means of the said probes and/or labels attached to one or more differentially methylated CpG position of FBLN1 SEQ ID NO: 426 in CD4 and CD8 T-lymphocytes. A person skilled in the art knows suitable methods for separation of labeled cells form unlabeled cells, tissue, or molecules.

Alternatively, for example but not limited to, a method for quantifying the number of CD4 and/or CD8 T-lymphocytes comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of FBLN1 SEQ ID NO: 426 of the provided sample. A person skilled in the art knows how to determine the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of CD4 and/or CD8 T-lymphocytes by comparing the determined one or more methylation levels of the sample with the respective herein provided one or more methylation levels of CD4 and/or CD8 T-lymphocytes and with at least one methylation level representing the correspondent tissue, group of cells, or cell of a healthy individual.

In a preferred embodiment, the markers of Table 8A and Table 9A are used to study the CD4 and/or CD8 T-lymphocyte infiltration in other tissues healthy or diseased. Infiltration of lymphocytes in healthy or diseased tissues is an indication of several diseases such immunological malignances or even in tumor development. The said markers represent a target for the development of molecular probes that coupled to any detection method (e.g. Fluorescent dye) allow the identification of these cells in histological preparations. For example but not limited to it, the differential methylation of HLA-DPB SEQ ID NO: 416 is used. According to Table 6, the differential methylation of HLA-DPB SEQ ID NO: 416 is marker for CD4 T-lymphocytes as well as for CD8 T-lymphocytes because the CpG dinucleotides of HLA-DPB SEQ ID NO: 416 are methylated within the range of 75-100% in CD4 and CD8 T-lymphocytes while other tissues show a different extend of methylation. Of course, corresponding markers can also be alternatively used. Thereby corresponding markers of HLA-DPB SEQ ID NO: 416 are, for example but not limited to, genomic DNA derived from or associated with HLA-DPB SEQ ID NO: 416; methylation specifically converted DNA derived from HLA-DPB SEQ ID NO: 416; mRNA, cDNA, protein, or peptide each of which derived at least in parts from HLA-DPB SEQ ID NO: 416. If the case may be, a person skilled in the art knows how to adjust the presented procedures.

Correspondingly, for example but not limited to it, a method for detection of infiltrated CD4 and/or CD8 T-lymphocytes comprises:
1. Providing of a sample, comprising genomic DNA;
2. Contacting the genomic DNA or a derivative of it with at least one probe which is specific for at least one differentially methylated CpG position of the marker HLA-DPB SEQ ID NO: 416. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art knows of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen).

According to a preferred embodiment, said method is performed in a histological manner. A person skilled in the art knows how to carried such a method. For example, but not limited to, the providing of the sample comprises the making of sample sections suitable for histological analysis.

In a preferred embodiment, the markers of Table 8A and Table 9A are used to identify, isolate and/or purify CD4 T-lymphocytes and/or CD8 T-lymphocytes, in particular from surrounding tissue infiltrated by the T-lymphocytes; from blood; and/or from other body fluids. For example but not limited to it, the differential methylation of APOBEC3B SEQ ID NO: 474 is used. According to Table 6, the differential methylation of APOBEC3B SEQ ID NO: 474 is a marker for CD4 T-lymphocytes as well as for CD8 T-lymphocytes because the CpG dinucleotides of APOBEC3B SEQ ID NO: 474 are methylated within the range of 0-25% in CD4 and CD8 T-lymphocytes while other tissues show a different extend of methylation. Of course, corresponding markers can also be alternatively used. Thereby corresponding markers of APOBEC3B SEQ ID NO: 474 are, for example but not limited to, genomic DNA derived from or associated with APOBEC3B SEQ ID NO: 474; methylation specifically converted DNA derived from APOBEC3B SEQ ID NO: 474; mRNA, cDNA, protein, or peptide each of which derived at least in parts from APOBEC3B SEQ ID NO: 474. If the case may be, a person skilled in the art knows how to adjust the presented procedures.

Correspondingly, for example but not limited to, a method for identifying CD4 T-lymphocytes and/or CD8 T-lymphocytes comprises:

1. Providing of a sample, the sample comprising genomic DNA.
2. Binding of at least one probe to one or more CpG positions within the sequence of APOBEC3B SEQ ID NO: 474 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying CD4 and/or CD8 T-lymphocytes by detecting the bound probes and/or their respective label(s). A person skilled in the art knows suitable methods for detection of said probes or labels.

For example but not limited to, a method for isolating and/or purifying CD4 and/or CD8 T-lymphocytes comprises in addition to the steps of the said method for identifying CD4 T-lymphocytes and/or CD8 T-lymphocytes:

3. Isolating and/or purifying of the identified CD4 and/or CD8 T-lymphocytes from the provided sample by means of the attached probes and their corresponding tags, respectively. A person skilled in the art knows suitable methods. Said methods are based on chemical, physical or biological properties of the attached probes or corresponding tags. For example but not limited to, the isolation is performed (i) by means of affinity cromatography, wherein the probe is attached to a tag that is recognized by an antibody immobilized on a column; (ii) by means of magnetic beads, wherein a magnetic bead is directly or indirectly bound to an attached probe and wherein a magnetic field is applied; or (iii) by means of fluorescent activated cell sorting, wherein the used tag is a fluorescent dye.

According to a preferred embodiment, the isolated or purified CD4 and/or CD8 T-lymphocytes are quantified by means of the attached probes and/or their corresponding tags. A person skilled in the art knows suitable methods. For example, but not limited to by cell counting manually or by automatic means.

In a preferred embodiment, the markers of Table 8A and Table 9A are used for the identification of an individual. Thereby at least two samples are used. One samples is obtained from an individual and another sample is a forensic sample, in particular traces of body cells, tissues or fluids for example but not limited to traces of blood and/or body fluids. This embodiment is of particular use in the field of forensic medicine or of legal medicine. As constituent of blood or body fluids, CD4 T-lymphocytes and CD8 T-lymphocytes are part of the mentioned traces. The said markers have the advantage of being stable over time because they are DNA based. In addition said markers have the advantage that they enable a highly detailed and accurate characterization of samples. Through this an unambiguous identification of an individual is enabled. For example but not limited to it, the differential methylation of GPX5 SEQ ID NO: 574 is used. According to Table 6, the differential methylation of GPX5 SEQ ID NO: 574 is a marker for CD4 T-lymphocytes as well as for CD8 T-lymphocytes because the CpG dinucleotides of GPX5 SEQ ID NO: 574 are methylated within the range of 0-25% in CD4 and CD8 T-lymphocytes while other tissues show a different extend of methylation. Of course, corresponding markers can also be alternatively used. Thereby corresponding markers of GPX5 SEQ ID NO: 574 are, for example but not limited to, genomic DNA derived from or associated with GPX5 SEQ ID NO: 574; methylation specifically converted DNA derived from GPX5 SEQ ID NO: 574; mRNA, cDNA, protein, or peptide each of which derived at least in parts from GPX5 SEQ ID NO: 574. If the case may be, a person skilled in the art knows how to adjust the presented procedures.

Correspondingly, for example but not limited to it, a method for the identification of an individual, comprises

1. Providing at least two samples. One sample is collected from an individual. Another sample is a forensic sample. Each of the provided samples comprises genomic DNA. The genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing each sample by determining the methylation state or the methylation level of at least one CpG position within the sequence of GPX5 SEQ ID NO: 574 of the provided samples. Thereby a profile is generated comprising the methylation information of all characterized CpG positions GPX5 SEQ ID NO: 574 of the respective samples. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3.Comparing the GPX5 profile of the forensic sample with one or more profiles of the samples collected from individual. An individual is identified wherein the GXP5 profile matches the profile of the sample of said individual. In alternative preferred embodiments, the forensic sample and the samples collected from individuals are collected or processed not at the same time.

In a preferred embodiment, the markers of Table 8A and Table 9A are used to diagnose the presence or absence of a disease. Thereby the number of CD4 T-lymphocytes, CD8 T-lymphocytes or both is quantified in normalized samples of healthy individuals. The determined number of CD4 T-lymphocytes, CD8 T-lymphocytes or both are then considered as indicative for healthy condition or a diseased condition with respect to an individual. Preferably, large amount of normalized samples are considered to generate reference values of CD4 T-lymphocytes, CD8 T-lymphocytes or both for a healthy condition and/or for one or more diseased conditions. The diseased condition can be any kind of diseased condition. Preferably, the diseased condition is a disease which causes a immune reaction. For example but not limited to the diseased condition is a cancer disease, a cell proliferation disease, or HIV. Preferably the total number of cells present in a sample is determined. The number of CD4 T-lymphocytes, CD8 T-lymphocytes or both are then normalized to the total number of cells. For example but not limited to it, the differential methylation of SYNE1 SEQ ID NO: 558 is used. According to Table 6, the differential methylation of SYNE1 SEQ ID NO: 558 is a marker for CD4 T-lymphocytes, for CD8 T-lymphocytes as well as sperm because the CpG dinucleotides of SYNE1 SEQ ID NO: 558 are methylated within the range of 75-100% in CD4 T-lymphocytes, CD8 T-lymphocytes and sperm while other tissues show a different extend of methylation. Because sperm can be easily morphologically distinguished from CD4 or CD8 T-lymphocytes, this marker can be used in the following described application. Of course, corresponding markers can also be alternatively used. Thereby corresponding markers of SYNE1 SEQ ID NO: 558 are, for example but not limited to, genomic DNA derived from or associated with SYNE1 SEQ ID NO: 558; methylation specifically converted DNA derived from SYNE1 SEQ ID NO: 558; mRNA, cDNA, protein, or peptide each of which derived at least in parts from SYNE1 SEQ ID NO: 558. If the case may be, a person skilled in the art knows how to adjust the presented procedures.

Correspondingly, for example but not limited to it, a method for diagnosing the absence or presence of a diseased or healthy condition for an individual, comprises
1. Providing a sample, comprising genomic DNA.
2. Determining the number of CD4 T-lymphocytes, CD8 T-lymphocytes or both by detecting the methylation status or level of at least one CpG position within the sequence of SYNE1 SEQ ID NO: 558 of the provided samples. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Normalizing the determined number of CD4 T-lymphocytes, CD8 T-lymphocytes or CD4 T-lymphocytes and CD8 T-lymphocytes. A person skilled in the art knows suitable methods for normalization. For example but not limited to the determined number of CD4 T-lymphocytes, CD8 T-lymphocytes or CD4 T-lymphocyte and CD8 T-lymphocytes is normalized to the number of total cell contained the provided sample.
   1. In a preferred embodiment, step 2 is performed in a histological, immunohistological and/or immunocytological manners. A person skilled in the art knows suitable methods for carrying out step 2 in histological, immunohistological and/or immunocytological manners for example but not limited to in situ hybridization, antibody stainings, or FACS. In another preferred embodiment, step 2 is performed by means of molecular biological methods. A person skilled in the art knows also suitable methods therefor for example but not limited to real time PCR based methods or hybridization based methods (Microarrays).

### Embryonic

In a preferred embodiment, the herein provided markers of Table 8B, Table 8C, Table 9B and Table 9C are used for the study, identification and/or quantification of fetal cells or fetal DNA circulating in maternal blood and/or amniotic fluid. During pregnancy cells and DNA from the fetus are continuously brought to the maternal blood stream as well as the amniotic fluid. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S100). For example but not limited to it, the differential methylation of CTA-384D8.15 SEQ ID NO: 509 is used. According to Table 6, the differential methylation of CTA-384D8.15 SEQ ID NO: 509 is a marker for embryonic liver because the CpG dinucleotides of CTA-384D8.15 SEQ ID NO: 509 are methylated within the range of 25-75% in embryonic liver while other tissues show a different extend of methylation. Of course, corresponding markers can also be alternatively used. Thereby corresponding markers of CTA-384D8.15 SEQ ID NO: 509 are, for example but not limited to, genomic DNA derived from or associated with CTA-384D8.15 SEQ ID NO: 509; methylation specifically converted DNA derived from CTA-384D8.15 SEQ ID NO: 509; mRNA, cDNA, protein, or peptide each of which derived at least in parts from CTA-384D8.15 SEQ ID NO: 509. If the case may be, a person skilled in the art knows how to adjust the presented procedures.

Correspondingly, for example but not limited to, a method for identifying fetal cells or fetal DNA comprises:

1. Providing of a sample, the sample comprising one or more cells or genomic DNA.
2. Binding of at least one probe to one or more CpG positions within the sequence of CTA-384D8.15 SEQ ID NO: 509 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying fetal cells or fetal genomic DNA by detecting the bound probes and/or their respective label(s). A person skilled in the art knows suitable methods for detection of said probes or labels.

In preferred embodiments, a probe of step 2 binds specifically with respect to the methylation status of 1, 2, 3, 4, 5, 6, 7, 8, 9, 10, 11, 12, 13, 14, 15, 16, 17, 18, 19, 20, 22, 25, or 30 CpG dinucleotides.

For example but not limited to, a method for isolating and/or purifying fetal cells or fetal genomic DNA comprises in addition to the steps of the said method for identifying fetal cells or fetal genomic DNA:

3. Isolating and/or purifying of the identified fetal cells or fetal genomic DNA from the provided sample by means of the attached probes and their corresponding tags, respectively. A person skilled in the art knows suitable methods. Said methods are based on chemical, physical or biological properties of the attached probes or corresponding tags. For example but not limited to, the isolation is performed (i) by means of affinity cromatography, wherein the probe (e.g. a nucleic acid) is directly or indirectly bound to a solid surface; (ii) by means of affinity cromatography, wherein the probe is attached to a tag that is recognized by an antibody immobilized on a column; (iii) by means of magnetic beads, wherein a magnetic bead is directly or indirectly bound to an attached probe and wherein a magnetic field is applied; or (iv) by means of fluorescent activated cell sorting, wherein the used tag is a fluorescent dye.

According to a preferred embodiment, the isolated or purified fetal cells or fetal genomic DNA are quantified by means of the attached probes and/or their corresponding tags. A person skilled in the art knows suitable methods. For example, but not limited to by cell counting manually or by automatic means. According to a preferred embodiment, the isolated or purified fetal genomic DNA is quantified by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method.

Alternatively, for example but not limited to, a method for quantifying the number of fetal cells or the amount of fetal genomic DNA comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of CTA-384D8.15 SEQ ID NO: 509 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of fetal cells or the amount of fetal genomic DNA by comparing the determined one or more methylation levels of the sample with the respective herein provided one or more methylation levels specific for embryonic liver and/or embryonic skeletal muscle and with at least one methylation level representing the correspondent tissue, group of cells, or cell comprising no placental DNA.

For example but not limited to it, a method for characterizing one or more fetal cells or fetal genomic DNA comprises

1. Providing of a sample comprising one or more fetal cells or fetal genomic DNA. The fetal cell(s) or the fetal genomic DNA are isolated for example but not limited to the methods described herein.
2. Characterizing said sample by determining the methylation state or the methylation level of at least one CpG position within the sequence of CTA-384D8.15 SEQ ID NO: 509 of the provided sample. Thereby a profile is generated comprising the methylation information of all characterized CpG positions of CTA-384D8.15 SEQ ID NO: 509 of the respective sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

In a preferred embodiment of step 1, the genomic DNA comprising fetal genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega). In a preferred embodiment the determined CTA-384D8.15 profile is compared with one or more CTA-384D8.15 profiles of obtained from other samples and/or with one or more CTA-384D8.15 reference profiles.

In a preferred embodiment, the herein provided markers of Table 8B, Table 8C, Table 9B and Table 9C are used for the study, identification and/or quantification of fetal cells or fetal DNA from amniocentesis and/or chorionic villus sampling. Said embodiment is of particular use in the field of prenatal diagnosis. Prenatal diagnosis procedures involve the study of fetal cells obtained by amniocentesis and chorionic villus biopsies. For example but not limited to it, the differential methylation of CRYBA4 SEQ ID NO: 476 is used. According to Table 6, the differential methylation of CRYBA4 SEQ ID NO: 476 is a marker for embryonic liver and embryonic skeletal muscle because the CpG dinucleotides of CRYBA4 SEQ ID NO: 476 are methylated within the range of 25-75% in embryonic liver or embryonic skeletal muscle while other tissues show a different extend of methylation. Of course, corresponding markers can also be alternatively used. Thereby corresponding markers of CRYBA4 SEQ ID NO: 476 are, for example but not limited to, genomic DNA derived from or associated with CRYBA4 SEQ ID NO: 476; methylation specifically converted DNA derived from CRYBA4 SEQ ID NO: 476; mRNA, cDNA, protein, or peptide each of which derived at least in parts from CRYBA4 SEQ ID NO: 476. If the case may be, a person skilled in the art knows how to adjust the presented procedures.

Correspondingly, for example but not limited to, a method for identifying fetal cells or fetal DNA comprises:

1. Providing of a sample, the sample comprising one or more cells or genomic DNA.
2. Binding of at least one probe to one or more CpG positions within the sequence of CRYBA4 SEQ ID NO: 476 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying fetal cells or fetal genomic DNA by detecting the bound probes and/or their respective label(s). A person skilled in the art knows suitable methods for detection of said probes or labels.

In preferred embodiments, a probe of step 2 binds specifically with respect to the methylation status of 1, 2, 3, 4, 5, 6, 7, 8, 9, 10, 11, 12, 13, 14, 15, 16, 17, 18, 19, 20, 22, 25, or 30 CpG dinucleotides.

For example but not limited to, a method for isolating and/or purifying fetal cells or fetal genomic DNA comprises in addition to the steps of the said method for identifying fetal cells or fetal genomic DNA:

3. Isolating and/or purifying of the identified fetal cells or fetal genomic DNA from the provided sample by means of the attached probes and their corresponding tags, respectively. A person skilled in the art knows suitable methods. Said methods are based on chemical, physical or biological properties of the attached probes or corresponding tags. For example but not limited to, the isolation is performed (i) by means of affinity cromatography, wherein the probe (e.g. a nucleic acid) is directly or indirectly bound to a solid surface; (ii) by means of affinity cromatography, wherein the probe is attached to a tag that is recognized by an antibody immobilized on a column; (iii) by means of magnetic beads, wherein a magnetic bead is directly or indirectly bound to an attached probe and wherein a magnetic field is applied; or (iv) by means of fluorescent activated cell sorting, wherein the used tag is a fluorescent dye.

According to a preferred embodiment, the isolated or purified fetal cells or fetal genomic DNA are quantified by means of the attached probes and/or their corresponding tags. A person skilled in the art knows suitable methods. For example, but not limited to by cell counting manually or by automatic means. According to a preferred embodiment, the isolated or purified fetal genomic DNA is quantified by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method.

Alternatively, for example but not limited to, a method for quantifying the number of fetal cells or the amount of fetal genomic DNA comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of CRYBA4 SEQ ID NO: 476 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of fetal cells or the amount of fetal genomic DNA by comparing the determined methylation levels of the sample with the respective herein provided methylation levels of embryonic liver and/or embryonic skeletal muscle.

For example but not limited to it, a method for characterizing one or more fetal cells or fetal genomic DNA comprises

1. Providing of a sample comprising one or more fetal cells or fetal genomic DNA. The fetal cell(s) or the fetal genomic DNA are isolated for example but not limited to the methods described herein.
2. Characterizing said sample by determining the methylation state or the methylation level of at least one CpG position within the sequence of CRYBA4 SEQ ID NO: 476 of the provided sample. Thereby a profile is generated comprising the methylation information of all characterized CpG positions of CRYBA4 SEQ ID NO: 476 of the respective sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

In a preferred embodiment of step 1, the genomic DNA comprising fetal genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).

In a preferred embodiment the determined CRYBA4 profile is compared with one or more CRYBA4 profiles of obtained from other samples and/or with one or more CRYBA4 reference profiles.

### Skin:

In a preferred embodiment, the herein provided markers of Tables 8D, G, I and Tables 9D, F, H are used for identifying individuals from traces of skin and/or adjacent tissues (such as hair, nail pieces, etc). This embodiment is of particular use in forensic medicine and/or legal medicine. Skin or skin adjacent tissue is usually used as study material in forensic and legal medicine. Preferably the markers provided in Table 8G and 9F are used because of the following reason. Keratinocytes constitute the external layer of the skin and therefore are the first cell type to be de-attached and a high number of these cells is expected in skin traces. Variations of one marker alone or in combination with other markers herein provided or not enable the accurate assessment of identity. For example but not limited to it, the differential methylation of NP_{—}612444.1 SEQ ID NO: 689 is used. According to Table 6, the differential methylation of NP_{—}612444.1 SEQ ID NO: 689 is a marker for keratinocytes as well as for sperm because the CpG dinucleotides of NP_{—}612444.1 SEQ ID NO: 689 are methylated within the range of 0-25% in keratinocytes and sperm while other tissues show a different extend of methylation. Of course, corresponding markers can also be alternatively used. Thereby corresponding markers of NP_{—}612444.1 SEQ ID NO: 689 are, for example but not limited to, genomic DNA derived from or associated with NP_{—}612444.1 SEQ ID NO: 689; methylation specifically converted DNA derived from NP_{—}612444.1 SEQ ID NO: 689; mRNA, cDNA, protein, or peptide each of which derived at least in parts from NP_{—}612444.1 SEQ ID NO: 689. If the case may be, a person skilled in the art knows how to adjust the presented procedures.

Correspondingly, for example but not limited to it, a method for the identification of an individual, comprises

1. Providing a sample, comprising skin, hair, nail pieces and/or adjacent tissue. Genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing the sample by determining the methylation state or the methylation level of at least one CpG position within the sequence of NP_{—}612444.1 SEQ ID NO: 689 of the provided sample. Thereby a profile is generated comprising the methylation information of all characterized CpG positions of NP_{—}612444.1 SEQ ID NO: 689 of the sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3.Comparing the determined NP_{—}612444.1 profile of the sample with one or more NP_{—}612444.1 profiles of individuals, the profiles obtained correspondingly or in a different manner. An individual is identified wherein the NP_{—}612444.1 profile matches the profile of an individual. In preferred embodiments, the forensic sample and the sample collected from an individual are collected and/or processed simultaneously or not.

In a preferred embodiment, the herein provided markers of Tables 8D, G, I and Tables 9D, F, H are used to characterize the skin, hair, nail, or adjacent tissue of an individual. For example but not limited to it, the differential methylation of SEQ ID NO: 640 (no gene associated) is used. According to Table 6, the differential methylation of SEQ ID NO: 640 is a marker for skin, hair, nail, or adjacent tissue because the CpG dinucleotides of SEQ ID NO: 640 are methylated within the range of 25-75% in keratinocytes, melanocytes and fibroblasts while other tissues show a different extend of methylation. Of course, corresponding markers can also be used alternatively. Thereby corresponding markers of SEQ ID NO: 640 are, for example but not limited to, genomic DNA derived from or associated with SEQ ID NO: 640; methylation specifically converted DNA derived from SEQ ID NO: 640; mRNA, cDNA, protein, or peptide each of which derived at least in parts from SEQ ID NO: 640. If the case may be, a person skilled in the art knows how to adjust the presented procedures.

Correspondingly, for example but not limited to, a method for characterizing skin, hair, nail and/or adjacent tissue comprises the characterization of at least keratinocytes, melanocytes, fibroblasts or combinations thereof, in addition comprising:

1. Providing a sample, comprising skin, hair, nail and/or adjacent tissue. Genomic DNA is purified from said samples, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing the sample by determining the methylation state or the methylation level of at least one CpG position within the sequence of SEQ ID NO: 640 of the provided sample. Thereby a profile is generated comprising the methylation information of all characterized CpG positions of SEQ ID NO: 640 of the sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

In a preferred embodiment, the herein provided markers of Tables 8D, G, 1 and Tables 9D, F, H are used to determine the composition of the skin, hair, nail, or adjacent tissue of an individual. Said composition being dependent from the content of at least one of the three major constituting cell types of the skin (fibroblasts, keratinocytes and melanocytes). For example but not limited to it, the differential methylation of SEQ ID NO: 644 (no gene associated), SEQ ID NO: 648 (no gene associated), PTPNS1 SEQ ID NO: 649, or combinations thereof are used. According to Table 6, the differential methylation of SEQ ID NO: 644 is a marker for melanocytes because the CpG dinucleotides of SEQ ID NO: 644 are methylated within the range of 0-25% in melanocytes while other tissues show a different extend of methylation. Of course, corresponding markers can also be used alternatively. Thereby corresponding markers of SEQ ID NO: 644 are, for example but not limited to, genomic DNA derived from or associated with SEQ ID NO: 644; methylation specifically converted DNA derived from SEQ ID NO: 644; mRNA, cDNA, protein, or peptide each of which derived at least in parts from SEQ ID NO: 644. According to Table 6, the differential methylation of SEQ ID NO: 648 is a marker for fibroblasts because the CpG dinucleotides of SEQ ID NO: 648 are methylated within the range of 0-25% in fibroblast while other tissues show a different extend of methylation. Of course, corresponding markers can also be used alternatively. Thereby corresponding markers of SEQ ID NO: 648 are, for example but not limited to, genomic DNA derived from or associated with SEQ ID NO: 648; methylation specifically converted DNA derived from SEQ ID NO: 648; mRNA, cDNA, protein, or peptide each of which derived at least in parts from SEQ ID NO: 648. According to Table 6, the differential methylation of PTPNS1 SEQ ID NO: 649 is a marker for keratinocytes because the CpG dinucleotides of PTPNS1 SEQ ID NO: 649 are methylated within the range of 0-25% in keratinocytes while other tissues show a different extend of methylation. Of course, corresponding markers can also be used alternatively. Thereby corresponding markers of PTPNS1 SEQ ID NO: 649 are, for example but not limited to, genomic DNA derived from or associated with PTPNS1 SEQ ID NO: 649; methylation specifically converted DNA derived from PTPNS1 SEQ ID NO: 649; mRNA, cDNA, protein, or peptide each of which derived at least in parts from PTPNS1 SEQ ID NO: 649. If the case may be, a person skilled in the art knows how to adjust the presented procedures.

Correspondingly, for example but not limited to, a method for quantifying the number of keratinocytes, fibroblast, melanocytes or combinations thereof comprises:

1. Providing of a sample, the sample comprising one or more cells or genomic DNA.
2. Binding of at least one probe to one or more CpG positions within the sequence of SEQ ID NO: 644, SEQ ID NO: 648, PTPNS1 SEQ ID NO: 649 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying keratinocytes, fibroblasts, melanocytes or combinations thereof by detecting the bound probes and/or their respective label(s). A person skilled in the art knows suitable methods for detection of said probes or labels.
4. Quantifying the isolated or purified cells by means of the attached probes and/or their corresponding tags. A person skilled in the art knows suitable methods. For example, but not limited to by cell counting manually or by automatic means. According to a preferred embodiment, the isolated or purified fetal genomic DNA is quantified by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method.

Alternatively, for example but not limited to, a method for quantifying the number of keratinocytes, fibroblast, melanocytes or combinations thereof comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of SEQ ID NO: 644, SEQ ID NO: 648, PTPNS1 SEQ ID NO: 649 or combinations thereof of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of keratinocytes, fibroblasts, melanocytes, or combinations thereof by comparing the determined methylation levels of the sample with the respective herein provided methylation levels of keratinocytes, fibroblasts or melanocytes.

In a preferred embodiment, the herein provided markers of Tables 8D, G, I and Tables 9D, F, H are used in the field of drugs. Said embodiment is of particular use for the development of drugs as well as for the treatment with drugs. The skin, hair, nail or adjacent tissue of an individual can be characterized by means of the provided markers of Tables 8D, 6, I and Tables 9D, F, H. This information can then be used to develop new drugs or to access already existing drugs with regard to skin, hair, nail etc. of an individual or to subgroups of individuals. These subgroups are for example but not limited to be characterized by a disease and/or a defined type of skin or hair, etc. The efficiency of said drugs i.e. the presence or absence of the desired effect is also characterized or monitored by means of the provided markers of Tables 8D, G, I and Tables 9D, F, H. For example but not limited to it, the differential methylation of SEQ ID NO: 773 (no gene associated) is used. According to Table 6, the differential methylation of SEQ ID NO: 773 is a marker for skin, hair, nail, or adjacent tissue because the CpG dinucleotides of SEQ ID NO: 773 are methylated within the range of 0-25% in keratinocytes, melanocytes and fibroblasts while other tissues show a different extend of methylation. Of course, corresponding markers can also be used alternatively. Thereby corresponding markers of SEQ ID NO: 773 are, for example but not limited to, genomic DNA derived from or associated with SEQ ID NO: 773; methylation specifically converted DNA derived from SEQ ID NO: 773; mRNA, cDNA, protein, or peptide each of which derived at least in parts from SEQ ID NO: 773. If the case may be, a person skilled in the art knows how to adjust the presented procedures.

Correspondingly, for example but not limited to, a method for developing a drug and/or for treating an individual with a drug comprises:

1. Providing a sample obtained from an individual comprising genomic DNA of keratinocytes, melanocytes, fibroblasts or combinations thereof. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing said sample by determining the methylation status or level of at least one CpG position within the sequence of SEQ ID NO: 773 of the provided sample. Thereby a SEQ ID NO: 773 profile specific for said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Treating said individual with a drug.
4. Providing a sample of said individual after drug treatment.
5. Characterizing said sample after drug treatment by determining the methylation status or level of at least one CpG position within the sequence of SEQ ID NO: 773 of the provided sample. Thereby a SEQ ID NO: 773 profile specific for said sample after drug treatment is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
6. Comparing the determined SEQ ID NO: 773 profiles with drug treatment and without drug treatment with each other.

According to a preferred embodiment, guidelines are drawn from the comparison for the further drug development. According to another preferred embodiment, guidelines are drawn from the comparison for the treatment of an individual with said drug.

In a preferred embodiment, the herein provided markers of Tables 8D, G, I and Tables 9D, F, H are used as prognostic and/or diagnostic markers for wound healing, in particular in the field of surgery procedures wherein the skin is affected. For example but not limited to it, the differential methylation of SLC35E4 SEQ ID NO: 751 is used. According to Table 6, the differential methylation of SLC35E4 SEQ ID NO: 751 is a marker for wound healing because the CpG dinucleotides of SLC35E4 SEQ ID NO: 751 are methylated within the range of 0-25% in keratinocytes, melanocytes and fibroblasts while other tissues show a different extend of methylation. Of course, corresponding markers can also be used alternatively. Thereby corresponding markers of SLC35E4 SEQ ID NO: 751 are, for example but not limited to, genomic DNA derived from or associated with SLC35E4 SEQ ID NO: 751; methylation specifically converted DNA derived from SLC35E4 SEQ ID NO: 751; mRNA, cDNA, protein, or peptide each of which derived at least in parts from SLC35E4 SEQ ID NO: 751. If the case may be, a person skilled in the art knows how to adjust the presented procedures.

Correspondingly, for example but not limited to, a method for prognosing or diagnosing would healing comprises:

1. Providing a sample obtained from an individual comprising genomic DNA of keratinocytes, melanocytes, fibroblasts or combinations thereof. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing said sample by determining the methylation status or level of at least one CpG position within the sequence of SLC35E4 SEQ ID NO: 751 of the provided sample. Thereby a SLC35E4 profile specific for said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Comparing the determined SLC35E4 profile with at least one reference profile obtained from the same individual and/or from one or more other individuals. Preferably, said reference profiles are obtained according to steps 1 and 2. Preferably, said reference profiles comprises pairs of profiles each pair being obtained from an individual. Thereby said pair consists of a first profile specific for the condition of healthy skin (no lesion of skin) and of a second profile specific for a healing state of the wound or affected skin area after lesion. A person skilled in the art knows to deduce the possible grade of scare building and or the time required for healing therefrom.

In a preferred embodiment, said method is a method for diagnosing wound healing wherein the determined SLC35E4 profile matches a reference profile. In another preferred embodiment, said method is a method for prognosing wound healing wherein the determined SLC35E4 profile matches a first reference profile and wherein a second reference profile exists which was obtained from the same individual as the first reference profile. Thereby the second reference profile was obtained after the first reference profile. Accordingly, the prognosis for the individual for whom the SLC35E4 profile was determined is the condition of wound healing characterized by the second reference profile. In preferred embodiments, at least 1, 2, 4, 6, 10, 15, 25 or 50 reference profiles are considered. The more reference profiles are considered the merrier is the diagnosis or prognosis.

### Liver

In a preferred embodiment, the herein provided markers of Tables 8H and Tables 9G are used for deducing the presence of absence of an event which affects the liver. For example but not limited to it, said event is at least one select from the group comprising liver cirrhosis; liver cancer; hepatitis A; hepatitis B; hepatitis C; healthy condition, recently or longer chemical, physical or biological exposure; recently or longer exposure to a drug, or alcohol; or treatment procedures. In the case the event is adverse, said event affecting the liver leads to the death of liver cells. In the case the event is benign, said event leads to a reduction of liver cell death. The genomic DNA of dead liver cells can then be found in the body fluids in particular in the blood of a affected individual. As an example but not limited to, the differential methylation of VARS SEQ ID NO: 415 is used. According to Table 6, the differential methylation of VARS SEQ ID NO: 415 is a marker for diseases affecting the liver because the CpG dinucleotides of VARS SEQ ID NO: 415 are methylated within the range of 25-75% in liver while other tissues show a different extend of methylation. Of course, corresponding markers can also be used alternatively. Thereby corresponding markers of VARS SEQ ID NO: 415 are, for example but not limited to, genomic DNA derived from or associated with VARS SEQ ID NO: 415; methylation specifically converted DNA derived from VARS SEQ ID NO: 415; mRNA, cDNA, protein, or peptide each of which derived at least in parts from VARS SEQ ID NO: 415. If the case may be, a person skilled in the art knows how to adjust the presented procedures.

Correspondingly, for example but not limited to, a method for detecting a liver affecting event comprises:

1. Providing a sample comprising genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega). Preferably said sample is a blood sample, plasma sample, or urine sample.
2. Determining the methylation level of at least one CpG position within the sequence of VARS SEQ ID NO: 415 of the provided sample. Thereby a VARS profile specific of said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Deducing the presence or absence of a liver affecting event from the comparison of the determined VARS profile with one or more VARS reference profiles. Said reference profiles are specific for a healthy condition or a condition specific for an event. In the case the determined VARS profile matches or is similar to a reference profile specific for an event, said liver affecting event is present. In case the determined VARS profiles is not similar to a reference profile specific for an event, said liver affecting event is absent.

In addition, for example but not limited to it, a method for detecting a liver affecting event comprises the quantification of the amount of free floating genomic DNA of liver cells. Said method comprises:
1. Providing of a sample, comprising genomic DNA;
2. Contacting the genomic DNA or a derivative of it with at least one probe which is specific for at least one differentially methylated CpG position of the marker VARS SEQ ID NO: 415. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art is aware of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen). A person skilled in the art knows further suitable methods for detection.
4. Quantifying the detection reaction in a manner so that the detected signal is indicative for the amount of probe or label and therewith for the number of dead liver cell. A person knows suitable methods for quantification.
5. Deducing the presence or absence of a liver affecting event from the comparison of the determined number of dead liver cells with reference numbers of dead liver cells that are specific for an event. In the case the determined number matches or is similar to a reference number specific for a healthy condition, a liver affecting event is absent. In the case the determined number matches or is similar to a reference number specific for a condition specific for an event, a liver affecting event is present.

Alternatively, for example but not limited to, a method for detecting a liver affecting event comprises the quantification of the amount of free floating genomic DNA of liver cells. Said method comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of VARS SEQ ID NO: 415 of the provided sample. A person skilled in the art knows how to determine the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of dead liver cells by comparing the determined one or more methylation levels of the sample with the respective herein provided one or more methylation levels of liver cells and with at least one methylation level representing the correspondent tissue, group of cells, or cell of a healthy individual.

In a preferred embodiment, the herein provided markers of Tables 8H and Tables 9G are used for deducing the sensitivity of an individual to alcohol. Alcohol consumption changes the DNA methylation status as reviewed by Poschl et al, 2004 (Poschl G, Stickel F, Wang X D, Seitz H K. Alcohol and cancer: genetic and nutritional aspects. Proc Nutr Soc. 2004 February; 63(1):65-71.). As an example but not limited to, the differential methylation of BMP7 SEQ ID NO: 684 is used. According to Table 6, the differential methylation of BMP7 SEQ ID NO: 684 is a marker for diseases affecting the liver because the CpG dinucleotides of BMP7 SEQ ID NO: 684 are methylated within the range of 25-75% in liver while other tissues show a different extend of methylation. Of course, corresponding markers can also be used alternatively. Thereby corresponding markers of BMP7 SEQ ID NO: 684 are, for example but not limited to, genomic DNA derived from or associated with BMP7 SEQ ID NO: 684; methylation specifically converted DNA derived from BMP7 SEQ ID NO: 684; mRNA, cDNA, protein, or peptide each of which derived at least in parts from BMP7 SEQ ID NO: 684. If the case may be, a person skilled in the art knows how to adjust the presented procedures.

Correspondingly, for example but not limited to, a method for deducing the sensitivity of an individual to alcohol comprises:

1. Providing a sample derived from said individual comprising genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega). Preferably said sample is a blood sample, plasma sample, or urine sample.
2. Determining the methylation level of at least one CpG position within the sequence of BMP7 SEQ ID NO: 684 of the provided sample. Thereby a BMP7 profile specific of said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Deducing the extend of a sensitivity of said individual to alcohol from the comparison of the determined BMP7 profile with one or more BMP7 reference profiles. The reference profiles are specific for different conditions of sensitivity. In the case the determined BMP7 profile matches or is similar to a reference profile, the sensitivity of said individual is identical or similar to the sensitivity for which the reference profile is specific for.

In a preferred embodiment, the sensitivity to alcohol is determined for a cell, group of cell, or tissue according to the above described procedure.

### Heart Muscle:

In a preferred embodiment, the herein provided markers of Tables 8E, Table 8F and Tables 9E are used for deducing the presence of absence of an event or condition affecting the heart. For example but not limited to it, said event or condition is at least one select from the group comprising heart failure; heart attack; athletic capacity; healthy condition; recently or longer chemical, physical or biological exposure; recently or longer exposure to a drug; or treatment procedure. In the case the event is adverse, said event or condition affecting the heart leads to death of heart cells. In the case the event is benign, said event leads to a reduction of heart cell death. The genomic DNA of dead heart cells can then be found in the body fluids in particular in the blood of an affected individual. As an example but not limited to, the differential methylation of TBC1D10A SEQ ID NO: 700 is used. According to Table 6, the differential methylation of TBC1D10A SEQ ID NO: 700 is a marker for diseases affecting the heart because the CpG dinucleotides of TBC1D10A SEQ ID NO: 700 are methylated within the range of 75-100% in heart while other tissues show a different extend of methylation. Of course, corresponding markers can also be used alternatively. Thereby corresponding markers of TBC1D10A SEQ ID NO: 700 are, for example but not limited to, genomic DNA derived from or associated with TBC1D10A SEQ ID NO: 700; methylation specifically converted DNA derived from TBC1D10A SEQ ID NO: 700; mRNA, cDNA, protein, or peptide each of which derived at least in parts from TBC1D10A SEQ ID NO: 700. If the case may be, a person skilled in the art knows how to adjust the presented procedures.

Correspondingly, for example but not limited to, a method for detecting a heart affecting event or condition comprises:

1. Providing a sample comprising genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega). Preferably said sample is a blood sample, plasma sample, or urine sample.
2. Determining the methylation level of at least one CpG position within the sequence of TBC1D10A SEQ ID NO: 700 of the provided sample. Thereby a TBC1D10A profile specific of said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Deducing the presence or absence of a heart affecting event or condition from the comparison of the determined TBC1D10A profile with one or more TBC1D10A reference profiles. Said reference profiles are specific for a healthy condition or a condition specific for an event. In case the determined TBC1D10A profile matches or is similar to a reference profile specific for an event or condition, the said heart affecting event or condition is present. In case the determined TBC1D10A profile is not similar to a reference profile specific for an event or condition, the said heart affecting event or condition is absent.

In addition, for example but not limited to it, a method for detecting a heart affecting event comprises the quantification of the amount of free floating genomic DNA of heart cells. Said method comprises:
1. Providing of a sample, comprising genomic DNA;
2. Contacting the genomic DNA or a derivative of it with at least one probe which is specific for at least one differentially methylated CpG position of the marker TBC1D10A SEQ ID NO: 700. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art is aware of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen). A person skilled in the art knows further suitable methods for detection.
4. Quantifying the detection reaction in a manner so that the detected signal is indicative for the amount of probe or label and therewith for the number of dead liver cell. A person knows suitable methods for quantification.
5. Deducing the presence or absence of a heart affecting event from the comparison of the determined number of dead heart cells with reference numbers of dead heart cells that are specific for an event. In the case the determined number matches or is similar to a reference number specific for an event or condition, said heart affecting event is present. In the case the determined number is not similar to a reference number specific for an event or condition, said heart affecting event is absent.

Alternatively, for example but not limited to, a method for detecting a heart affecting event comprises the quantification of the amount of free floating genomic DNA of heart cells. Said method comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of TBC1D10A SEQ ID NO: 700 of the provided sample. A person skilled in the art knows how to determine the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of dead heart cells by comparing the determined one or more methylation levels of the sample with the respective herein provided one or more methylation levels of heart cells and with at least one methylation level representing the correspondent tissue, group of cells, or cell of a healthy individual.

### Placenta:

In a preferred embodiment, the herein provided markers of Table 8J and Table 9I are used for the study, monitoring, identification and/or quantification of placental cells or placental DNA circulating in maternal blood and/or amniotic fluid. Placenta constitute an extra-embryonic fetal tissue and as such, it shares many genetic characteristics with the fetal tissue. Therefore, cells from the placenta as well as DNA from placental cells can surrogate fetal cells and fetal DNA for diagnostic means. Fetal cells and fetal DNA have a diagnostic potential in monitoring the health status of the fetus as reviewed by Bianchi D, 2004 (Bianchi D W. Circulating fetal DNA: its origin and diagnostic potential-a review. Placenta. 2004 April; 25 Suppl A:S93-S100). During pregnancy placenta cells are de-attached and brought to the maternal blood stream as well as the amniotic fluid. For example but not limited to it, the differential methylation of PRAME SEQ ID NO: 419 is used. According to Table 6, the differential methylation of PRAME SEQ ID NO: 419 is a marker for placenta because the CpG dinucleotides of PRAME SEQ ID NO: 419 are methylated within the range of 0-25% in placenta while other tissues show a different extend of methylation. Of course, corresponding markers can also be alternatively used. Thereby corresponding markers of PRAME SEQ ID NO: 419 are, for example but not limited to, genomic DNA derived from or associated with PRAME SEQ ID NO: 419; methylation specifically converted DNA derived from PRAME SEQ ID NO: 419; mRNA, cDNA, protein, or peptide each of which derived at least in parts from PRAME SEQ ID NO: 419. If the case may be, a person skilled in the art knows how to adjust the presented procedures.

Correspondingly, for example but not limited to, a method for identifying one or more placental cells or placental DNA comprises:

1. Providing of a sample, the sample comprising one or more cells or genomic DNA.
2. Binding of at least one probe to one or more CpG positions within the sequence of PRAME SEQ ID NO: 419 of the provided sample. Thereby a probe binds specifically with respect to the methylation status of said one or more CpG positions. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying placental cells or placental genomic DNA by detecting the bound probes and/or their respective label(s). A person skilled in the art knows suitable methods for detection of said probes or labels.

In preferred embodiments, a probe of step 2 binds specifically with respect to the methylation status of 1, 2, 3, 4, 5, 6, 7, 8, 9, 10, 11, 12, 13, 14, 15, 16, 17, 18, 19, 20, 22, 25, or 30 CpG dinucleotides.

For particular applications it is necessary to isolate or purify placental cell or placental genomic DNA. Accordingly, as an example but not limited to, a method for isolating and/or purifying placental cells or placental genomic DNA comprises in addition to the steps of the said method for identifying placental cells or placental genomic DNA:

3. Isolating and/or purifying of the identified placental cells or placental genomic DNA from the provided sample by means of the attached probes and their corresponding tags, respectively. A person skilled in the art knows suitable methods. Said methods are based on chemical, physical or biological properties of the attached probes or corresponding tags. For example but not limited to, the isolation is performed (i) by means of affinity cromatography, wherein the probe (e.g. a nucleic acid) is directly or indirectly bound to a solid surface; (ii) by means of affinity cromatography, wherein the probe is attached to a tag that is recognized by an antibody immobilized on a column; (iii) by means of magnetic beads, wherein a magnetic bead is directly or indirectly bound to an attached probe and wherein a magnetic field is applied; or (iv) by means of fluorescent activated cell sorting, wherein the used tag is a fluorescent dye.

According to a preferred embodiment, the isolated or purified placental cells or placental genomic DNA are quantified by means of the attached probes and/or their corresponding tags. A person skilled in the art knows suitable methods. For example, but not limited to by cell counting manually or by automatic means. According to a preferred embodiment, the isolated or purified placental genomic DNA is quantified by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method.

Alternatively, for example but not limited to, a method for quantifying the number of placental cells or the amount of placental genomic DNA comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of PRAME SEQ ID NO: 419 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of placental cells or the amount of placental genomic DNA by comparing the determined one or more methylation levels of the sample with the respective herein provided one or more methylation levels specific for placenta and with at least one methylation level representing the correspondent tissue, group of cells, or cell comprising no placental DNA.

For example but not limited to it, a method for characterizing one or more placental cells or placental genomic DNA comprises

1. Providing of a sample comprising one or more placental cells or placental genomic DNA. The placental cell(s) or the placental genomic DNA are isolated for example but not limited to the methods described herein.
2. Characterizing said sample by determining the methylation state or the methylation level of at least one CpG position within the sequence of PRAME SEQ ID NO: 419 of the provided sample. Thereby a profile is generated comprising the methylation information of all characterized CpG positions of PRAME SEQ ID NO: 419 of the respective sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

In a preferred embodiment of step 1, the genomic DNA comprising placental genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).

In a preferred embodiment, the determined PRAME profile is compared with one or more PRAME profiles obtained from other samples of the same individual and/or with one or more PRAME reference profiles of a normal pregnancy.

In a preferred embodiment, the herein provided markers of Table 8J and Table 9I are used for to the monitoring of embryonic development or the monitoring of placental development, in particular of extra-embryonic tissue or of interaction of extra-embryonic tissue with maternal placental tissue. Said embodiment comprises one or more of said methods for identifying one or more placental cells or placental DNA; one or more of said methods for isolating and/or purifying one or more placental cells or placental genomic DNA; one or more of said methods for quantifying the number of placental cells or the amount of placental genomic DNA; one or more of said methods for characterizing one or more placental cells or placental genomic DNA; or combinations thereof. application is for example but not limited

In a preferred embodiment, the herein provided markers of Table 8J and Table 9I are used for the study, monitoring, identification and/or quantification of placental cells in regenerative medicine, in particular in the field of tissue engineering. Therefore the above described methods (a method for identifying one or more placental cells or placental DNA; a method for isolating and/or purifying one or more placental cells or placental genomic DNA; a method for quantifying the number of placental cells or the amount of placental genomic DNA; a method for characterizing one or more placental cells or placental genomic DNA; a method for monitoring one or more placental cells) or combinations thereof are used. Thereby the said methods are applied to placental cells or to cells derived from placental cells. Furthermore the methods are applied in particular before and after storage, before and after cell differentiation, before and after cell proliferation, before and after cell culture expansion, and before and after tissue expansion as well as before and after transplantation. For example but not limited to, the differential methylation of GPR24 SEQ ID NO: 436 is used. According to Table 6, the differential methylation of GPR24 SEQ ID NO: 436 is a marker for placenta because the CpG dinucleotides of GPR24 SEQ ID NO: 436 are methylated within the range of 25-75% in placenta while other tissues show a different extend of methylation. Said methods (a method for identifying one or more placental cells or placental DNA; a method for isolating and/or purifying one or more placental cells or placental genomic DNA; a method for quantifying the number of placental cells or the amount of placental genomic DNA; a method for characterizing one or more placental cells or placental genomic DNA; a method for monitoring one or more placental cells) are carried out as described above, wherein PRAME SEQ ID NO: 419 is substituted by GPR24 SEQ ID NO: 436. Of course, corresponding markers can also be alternatively used. Thereby corresponding markers of GPR24 SEQ ID NO: 436 are, for example but not limited to, genomic DNA derived from or associated with GPR24 SEQ ID NO: 436; methylation specifically converted DNA derived from GPR24 SEQ ID NO: 436; mRNA, cDNA, protein, or peptide each of which derived at least in parts from GPR24 SEQ ID NO: 436. If the case may be, a person skilled in the art knows how to adjust the said methods.

### Sperm:

The herein provided markers of Table 8L are used for diagnosing a male infertility related disease. A major cause of male infertility is either a low amount of sperm cells (spermatozoa) in the ejaculate (oligospermia) or a complete lack of sperm cells (spermatozoa) in the ejaculate (azoospermia). Thus, methods for the quantification of sperm cells are widely used in diagnosis of male infertility. In addition methylation analysis of sperm cells can be used as a tool to access the viability of the said. For example but not limited to, the differential methylation of GAL3ST1 SEQ ID NO: 437 is used. According to Table 6, the differential methylation of GAL3ST1 SEQ ID NO: 437 is a marker for sperm because the CpG dinucleotides of GAL3ST1 SEQ ID NO: 437 are methylated within the range of 0-25% in sperm while other tissues show a different extend of methylation. Of course, corresponding markers can also be alternatively used. Thereby corresponding markers of GAL3ST1 SEQ ID NO: 437 are, for example but not limited to, genomic DNA derived from or associated with GAL3ST1 SEQ ID NO: 437; methylation specifically converted DNA derived from GAL3ST1 SEQ ID NO: 437; mRNA, cDNA, protein, or peptide each of which derived at least in parts from GAL3ST1 SEQ ID NO: 437. If the case may be, a person skilled in the art knows how to adjust the presented procedures.

Correspondingly, for example but not limited to, a method for diagnosing a male infertility related disease comprises the quantification and/or characterization of sperm cells in the ejaculate.

For example, but not limited to, a method for characterization of sperm cells in the ejaculate comprises:

1. Providing of a sample comprising ejaculate or genomic DNA derived from an ejaculate. The genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing said sample by determining the methylation state or the methylation level of at least one CpG position within the sequence of GAL3ST1 SEQ ID NO: 437 of the provided sample. Thereby a profile is generated comprising the methylation information of all characterized CpG positions of GAL3ST1 SEQ ID NO: 437 of the respective sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.

According to a preferred embodiment, the determined GAL3ST1 profile of a provided sample is compared with at least one GAL3ST1 reference profile. Said reference profile is either obtained from a different individual or from the same individual. The reference profile is characterized in that it is specific for a defined state of viability of sperm cells. In a preferred embodiment several reference profiles are used each of which is specific for a defined state of viability of sperm cells in the ejaculate and therefore for a defined value of infertility or fertility. In case a determined GAL3ST1 profile matches or is similar to a reference profile, it is deduced that the viability of sperm cells of the correspondent individual is characterized by the said defined state of viability of the reference.

Alternatively, for example, but not limited to, a method for characterizing sperm cells in an ejaculate comprises:
1. Providing of a sample comprising ejaculate or genomic DNA derived from an ejaculate.
2. Binding of at least one probe to one or more CpG positions within the sequence of GAL3ST1 SEQ ID NO: 437 of the provided sample. Thereby a probe binds specifically with respect to the methylation status of said one or more CpG positions creating a characteristic binding pattern. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof, or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.

According to a preferred embodiment, the determined GAL3ST1 binding pattern is compared with at least one GAL3ST1 reference binding pattern. Said reference binding pattern is either obtained from a different individual or from the same individual. The reference binding pattern is characterized in that it is specific for a defined state of viability of sperm cells. In a preferred embodiment several reference binding patterns are used each of which is specific for a defined state of viability of sperm cells in the ejaculate and therefore for a defined value of infertility or fertility. In case a determined GAL3ST1 binding pattern matches or is similar to a reference binding pattern, it is deduced that the viability of sperm cells of the correspondent individual is characterized by the said defined state of viability of the reference.

For example, but not limited to, a method for quantification of sperm cells in the ejaculate comprises the identification of sperm cells. For example, but not limited to, a method for identification of sperm cells comprises:

1. Providing of a sample comprising ejaculate or genomic DNA derived from an ejaculate.
2. Binding of at least one probe to one or more CpG positions within the sequence of GAL3ST1 SEQ ID NO: 437 of the provided sample. Thereby a probe binds specifically with respect to the methylation status of said one or more CpG positions creating a characteristic binding pattern. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof; or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying sperm cells or sperm genomic DNA by detecting the bound probes and/or their respective label(s). A person skilled in the art knows suitable methods for detection of said probes or labels.

In preferred embodiments, a probe of step 2 binds specifically with respect to the methylation status of 1, 2, 3, 4, 5, 6, 7, 8, 9, 10, 11, 12, 13, 14, 15, 16, 17, 18, 19, 20, 22, 25, or 30 CpG dinucleotides.

The said method for quantification of sperm cells in the ejaculate comprising the identification of sperm cells additional comprises:

Quantifying the identified sperm cells by counting or quantifying the labeled sperm genomic DNA by quantifying the bound probes and/or their respective label(s). A person skilled in the art knows suitable methods for quantification and/or counting.

Alternatively, for example but not limited to, a method for quantification of sperm cells in the ejaculate comprises:

1. Providing of a sample comprising ejaculate or genomic DNA derived from an ejaculate. The genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of GAL3ST1 SEQ ID NO: 437 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of sperm cells or the amount of sperm genomic DNA by comparing the determined one or more methylation levels of the sample with the respective herein provided one or more methylation levels specific for sperm and with at least one methylation level representing an ejaculate comprising no sperm cell or a defined amount of sperm cells.

For example but not limited to, a method for diagnosing fertility or infertility for a male individual comprises:
1. Quantifying the amount of sperm cells in the ejaculate of said individual.
2. Comparing said amount with at least one reference value. The reference value is specific for a defined fertility or infertility state. In case the quantified amount of sperm cells matches or is similar to the reference value, it is deduced that the fertility or infertility of said individual has the same fertility or infertility which is specific for the reference value.

In a preferred embodiment, the herein provided markers of Table 8L are used for increasing the fertility of a male individual. As said above male fertility is often limited by the amount of sperm cells in the ejaculate. Thus, male fertility can be enhanced by enriching, isolating or purifying sperm cells. For example but not limited to, the differential methylation of APOL4 SEQ ID NO: 486 is used. According to Table 6, the differential methylation of APOL4 SEQ ID NO: 486 is a marker for sperm because the CpG dinucleotides of APOL4 SEQ ID NO: 486 are methylated within the range of 75-100% in sperm while other tissues show a different extend of methylation. Of course, corresponding markers can also be alternatively used. Thereby corresponding markers of APOL4 SEQ ID NO: 486 are, for example but not limited to, genomic DNA derived from or associated with APOL4 SEQ ID NO: 486; methylation specifically converted DNA derived from APOL4 SEQ ID NO: 486; mRNA, cDNA, protein, or peptide each of which derived at least in parts from APOL4 SEQ ID NO: 486. If the case may be, a person skilled in the art knows how to adjust the presented procedures.

Correspondingly, for example but not limited to, a method for increasing the fertility of a male individual comprises:

1. Identification of sperm cells in an ejaculate by means of the above described method for identification of sperm cells, wherein GAL3ST1 SEQ ID NO: 437 is substitutes by APOL4 SEQ ID NO: 486.
2. Enriching, isolating or purifying sperm cells by means of the attached probes and their corresponding tags, respectively. A person skilled in the art knows suitable methods. Said methods are based on chemical, physical or biological properties of the attached probes or corresponding tags. For example but not limited to, the isolation is performed (i) by means of affinity cromatography, wherein the probe (e.g. a nucleic acid) is directly or indirectly bound to a solid surface; (ii) by means of affinity cromatography, wherein the probe is attached to a tag that is recognized by an antibody immobilized on a column; (iii) by means of magnetic beads, wherein a magnetic bead is directly or indirectly bound to an attached probe and wherein a magnetic field is applied; or (iv) by means of fluorescent activated cell sorting, wherein the used tag is a fluorescent dye.

In a preferred embodiment, the herein provided markers of Table 8L are used for assisted fertilization procedures. Assisted fertilization procedures are for example but not limited to intracytoplasmic sperm injection (ICSI) or in vitro fertilization (IVF). All assisted fertilization procedures require the management of sperm cells prior to the procedure. Such management comprises at least the characterization, identification, quantification, enrichment, isolation, purification of sperm cells or combinations thereof. Therefore the above described methods for characterizing, identifying, quantifying, enriching, isolating and purifying sperm cells are applied.

In a preferred embodiment, the herein provided markers of Table 8L are used in the fields of forensic and/or legal medicine. By use of the said markers it is possible to determine the presence or absence of sperm in a sample. Furthermore, it is possible to identify an individual by use of said markers. For example but not limited to, the differential methylation of TCN2 SEQ ID NO: 470 is used. According to Table 6, the differential methylation of TCN2 SEQ ID NO: 470 is a marker for sperm because the CpG dinucleotides of TCN2 SEQ ID NO: 470 are methylated within the range of 75-100% in sperm while other tissues show a different extend of methylation. Of course, corresponding markers can also be alternatively used. Thereby corresponding markers of TCN2 SEQ ID NO: 470 are, for example but not limited to, genomic DNA derived from or associated with TCN2 SEQ ID NO: 470; methylation specifically converted DNA derived from TCN2 SEQ ID NO: 470; mRNA, cDNA, protein, or peptide each of which derived at least in parts from TCN2 SEQ ID NO: 470. If the case may be, a person skilled in the art knows how to adjust the presented procedures.

Correspondingly, for example but not limited to, a method for identifying sperm cells in a sample and/or for identifying an individual comprises:
1. Providing a sample comprising genomic DNA.
2. Binding of at least one probe to one or more CpG positions within the sequence of TCN2 SEQ ID NO: 470 of the provided sample. Thereby a probe binds specifically with respect to the methylation status of said one or more CpG positions and/or with respect to the methylation status of said one or more CpG positions and an individual. A probe is either a protein, peptide, nucleic acid, RNA or DNA for example but not limited to, an antibody specific for 5-methylcytosine (e.g. AbCAM Cat. No. ab1884); a methyl-binding protein such as the proteins MeCP2, MBD1, MBD2, MBD4, Kaiso or any domain thereof, or a nucleic acid probe that is specific for the methylated sequence. According to some preferred embodiments, the said probe(s) are labeled with a tag suitable for detection of the probe, isolation of one or more cells, and/or purification of one or more cells. A person skilled in the art knows suitable methods to carry out this step.
3. Identifying sperm cells or sperm genomic DNA or identifying sperm cells or sperm genomic DNA of an individual by detecting the bound probes and/or their respective label(s). A person skilled in the art knows suitable methods for detection of said probes or labels.

In addition, for example but not limited to, an alternative method for identifying an individual comprises:

1. Providing of a sample comprising genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of TCN2 SEQ ID NO: 470 of the provided sample. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Comparing the determined methylation level(s) of said sample with respective TCN2 SEQ ID NO: 470 methylation level(s) of at least one sample obtained from at least one candidate individual. Wherein the determined methylation level(s) matches or is similar to the respective methylation level(s) of an individual, it its deduced that the provided sample comprises sperm genomic DNA or sperm cells of said individual. Thereby an individual is identified.

### Skeletal Muscle

In a preferred embodiment, the herein provided markers of Table 8F, 8K and Table 9J are used for characterizing the efficiency of skeletal muscle cells. This embodiment is of particular value in the field of sports medicine. For example but not limited to, the differential methylation of CARD10 SEQ ID NO: 498 is used. According to Table 6, the differential methylation of CARD10 SEQ ID NO: 498 is a marker for skeletal muscle because the CpG dinucleotides of CARD10 SEQ ID NO: 498 are methylated within the range of 0-25% in skeletal muscle while other tissues show a different extend of methylation. Of course, corresponding markers can also be alternatively used. Thereby corresponding markers of CARD10 SEQ ID NO: 498 are, for example but not limited to, genomic DNA derived from or associated with CARD10 SEQ ID NO: 498; methylation specifically converted DNA derived from CARD10 SEQ ID NO: 498; mRNA, cDNA, protein, or peptide each of which derived at least in parts from CARD10 SEQ ID NO: 498. If the case may be, a person skilled in the art knows how to adjust the presented procedures.

Correspondingly, for example but not limited to, a method for characterizing the efficiency of a skeletal muscle comprises:

1. Providing of a sample comprising genomic DNA of a skeletal muscle. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing the provided sample by Determining the methylation level of at least one CpG position within the sequence of CARD10 SEQ ID NO: 498 of the provided sample. Thereby a CARD 10 profile is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Comparing the generated CARD10 profile with at least one CARD10 reference profile. Thereby each reference profile is characteristic for a defined efficiency. Wherein the determined CARD10 profile matches or is similar to a CARD10 reference profile, the skeletal muscle from whom the analyzed sample is provided has the same efficiency as the said reference.

In a preferred embodiment, the herein provided markers of Table 8F, 8K and Table 9J are used for identifying fully differentiated muscle cells in cell culture. This is of particular value in the field of tissue engineering. Muscle cells are generate in cell culture by cultivation and differentiation of muscle cell progenitor cells. Fully differentiated skeletal muscle cells can be identified by means of the provided markers of Table 8F, 8K and Table 9J. For example but not limited to, the differential methylation of HTF9C_HUMAN SEQ ID NO: 500 is used. According to Table 6, the differential methylation of HTF9C_HUMAN SEQ ID NO: 500 is a marker for skeletal muscle because the CpG dinucleotides of HTF9C_HUMAN SEQ ID NO: 500 are methylated within the range of 25-75% in skeletal muscle while other tissues show a different extend of methylation. Of course, corresponding markers can also be alternatively used. Thereby corresponding markers of HTF9C_HUMAN SEQ ID NO: 500 are, for example but not limited to, genomic DNA derived from or associated with HTF9C_HUMAN SEQ ID NO: 500; methylation specifically converted DNA derived from HTF9C_HUMAN SEQ ID NO: 500; mRNA, cDNA, protein, or peptide each of which derived at least in parts from HTF9C_HUMAN SEQ ID NO: 500. If the case may be, a person skilled in the art knows how to adjust the presented procedures.

Correspondingly, for example but not limited to, a method for identifying fully in vitro differentiated muscle cell comprises:

1. Providing of a sample comprising genomic DNA of a skeletal muscle. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing the provided sample by Determining the methylation level of at least one CpG position within the sequence of HTF9C_HUMAN SEQ ID NO: 500 of the provided sample. Thereby a HTF9C_HUMAN profile is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Comparing the generated HTF9C_HUMAN profile with a HTF9C_HUMAN reference profile which is characteristic for a fully differentiated muscle cell. Wherein the determined HTF9C_HUMAN profile matches or is similar to the reference profile, the analyzed skeletal muscle cell is considered as fully differentiated.

In a preferred embodiment, the herein provided markers of Table 8F, 8K and Table 9J are used for diagnosing muscle cell associated diseases, in particular disease which are characterized by a death of muscle cells like muscular distrophy. The DNA of dead muscle cells is found in body fluids such as blood or urine. This DNA is identified by means of the herein provided markers of Table 8F, 8K and Table 9J. For example but not limited to, the differential methylation of EYA2 SEQ ID NO: 678 is used. According to Table 6, the differential methylation of EYA2 SEQ ID NO: 678 is a marker for skeletal muscle because the CpG dinucleotides of EYA2 SEQ ID NO: 678 are methylated within the range of 25-75% in skeletal muscle while other tissues show a different extend of methylation. Of course, corresponding markers can also be alternatively used. Thereby corresponding markers of EYA2 SEQ ID NO: 678 are, for example but not limited to, genomic DNA derived from or associated with EYA2 SEQ ID NO: 678; methylation specifically converted DNA derived from EYA2 SEQ ID NO: 678; mRNA, cDNA, protein, or peptide each of which derived at least in parts from EYA2 SEQ ID NO: 678. If the case may be, a person skilled in the art knows how to adjust the presented procedures.

Correspondingly, for example but not limited to, a method for diagnosing muscle cell associated diseases comprises:

1. Providing of a sample derived from blood or urine, the sample comprising genomic DNA. The genomic DNA is isolated/purified from the provided sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Characterizing the sample by Determining the methylation level of at least one CpG position within the sequence of EYA2 SEQ ID NO: 678 of the provided sample. Thereby a EAY2 profile is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Deducing the presence or absence of a muscle associated disease from comparison of the determined EYA2 profile with at least one EYA2 reference profile. A EYA2 reference profile comprises the same position(s) as the determined profile. In addition a EYA2 profile is specific for either skeletal muscle cells (as herein provided by Table 8F, 8K and Table 9J), blood or urine comprising no skeletal muscle cell DNA, or blood or urine derived from a healthy individual.

### CD8 T-Lymphocytes

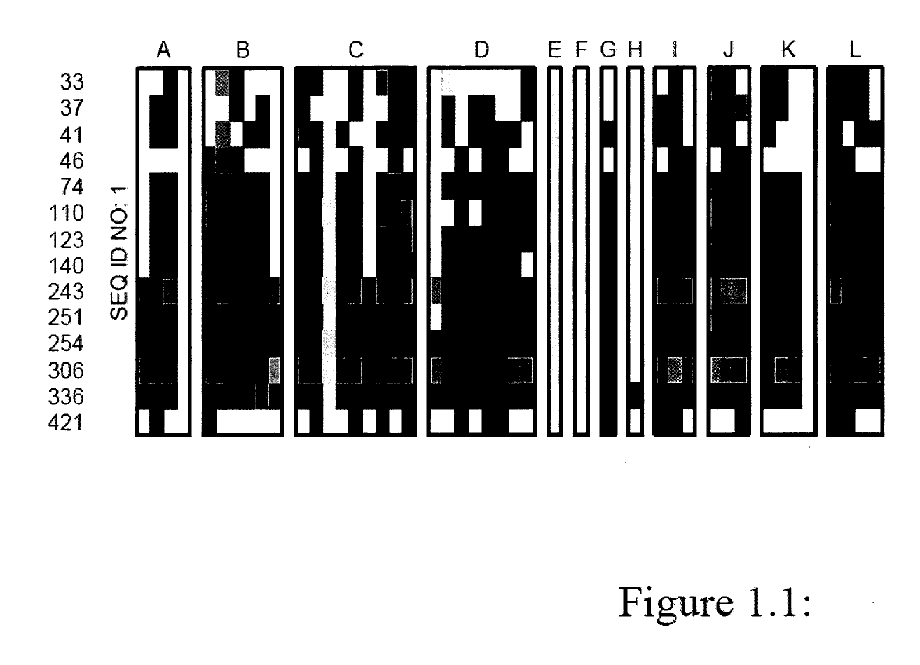

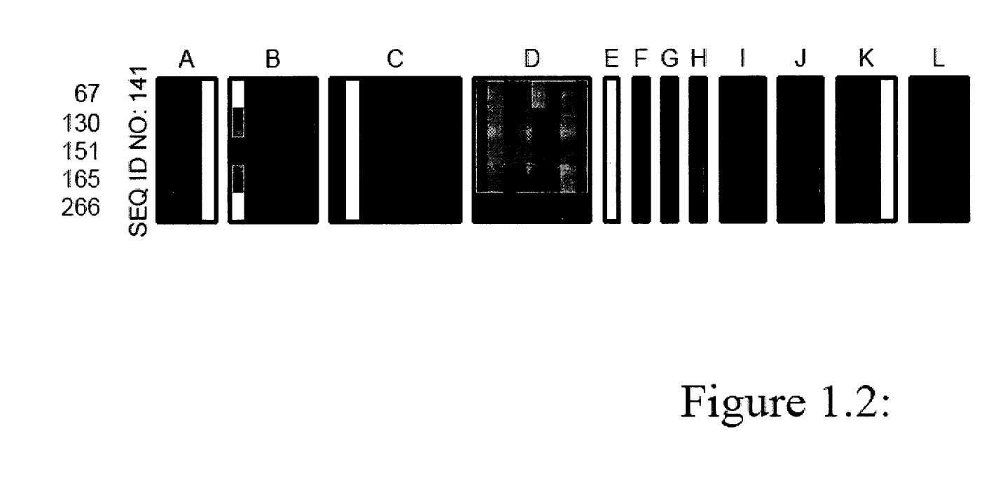

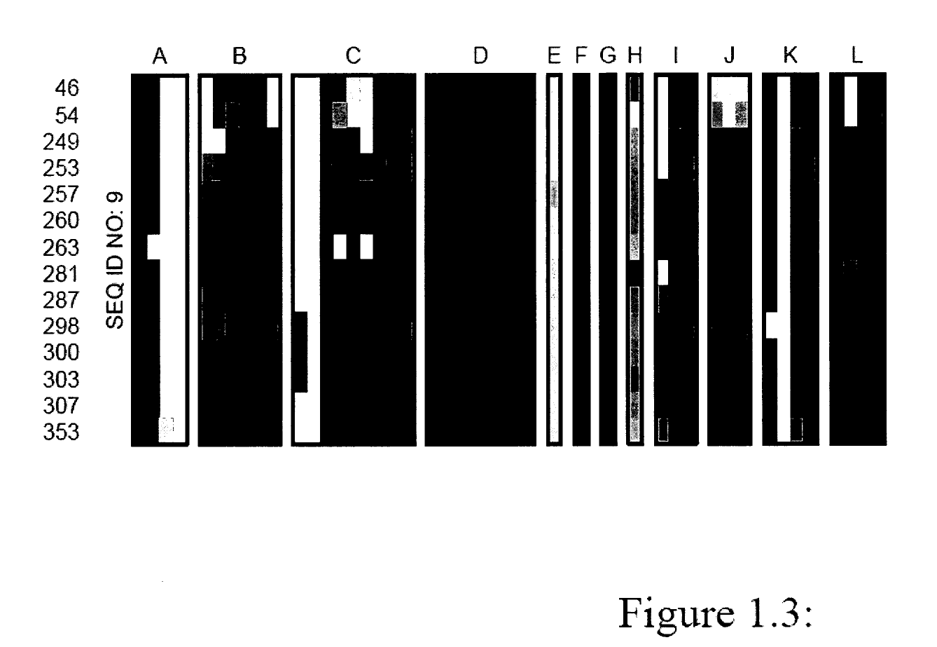

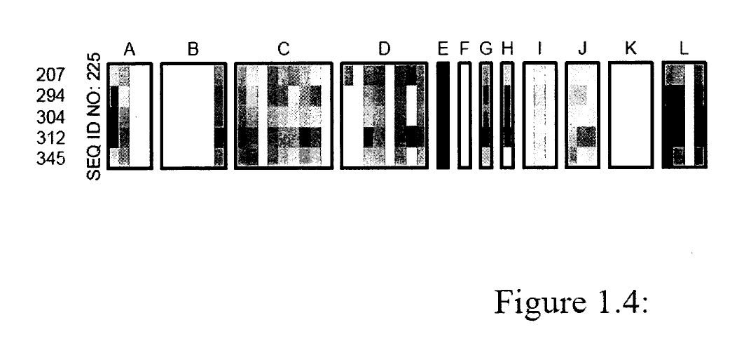

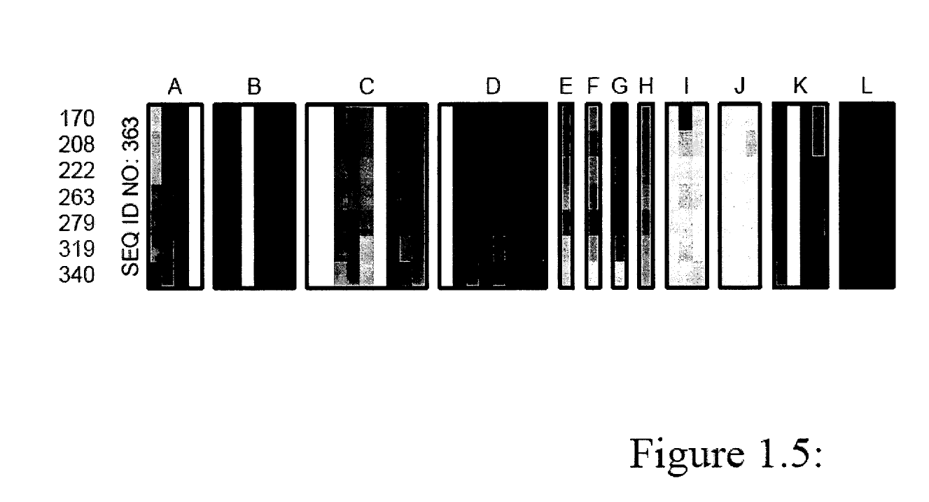

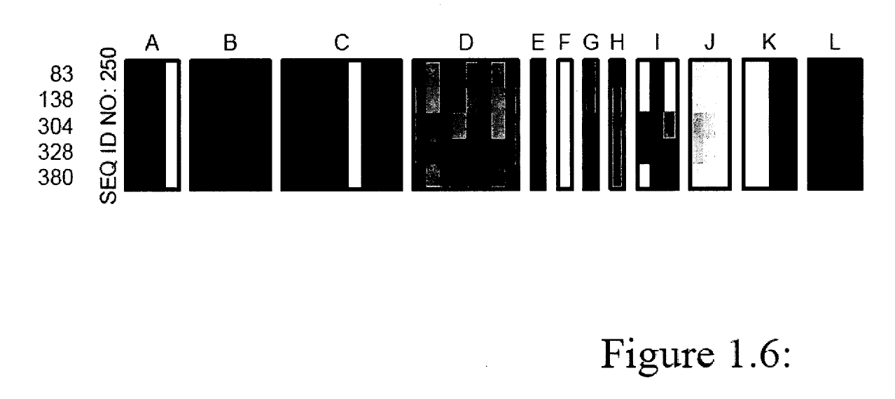

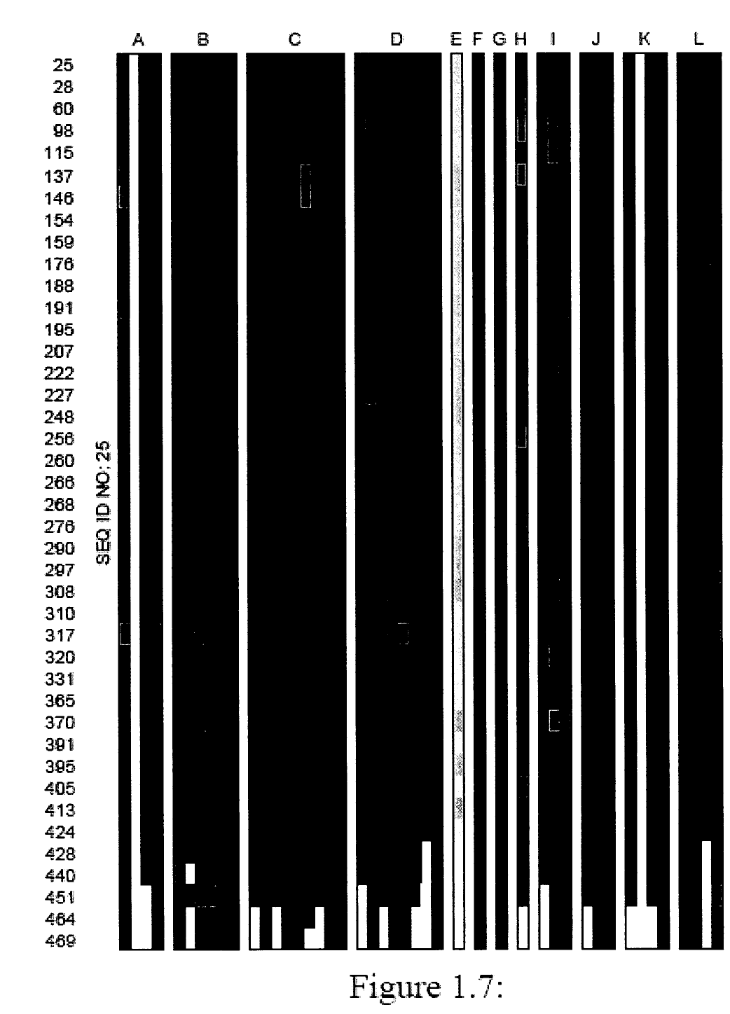

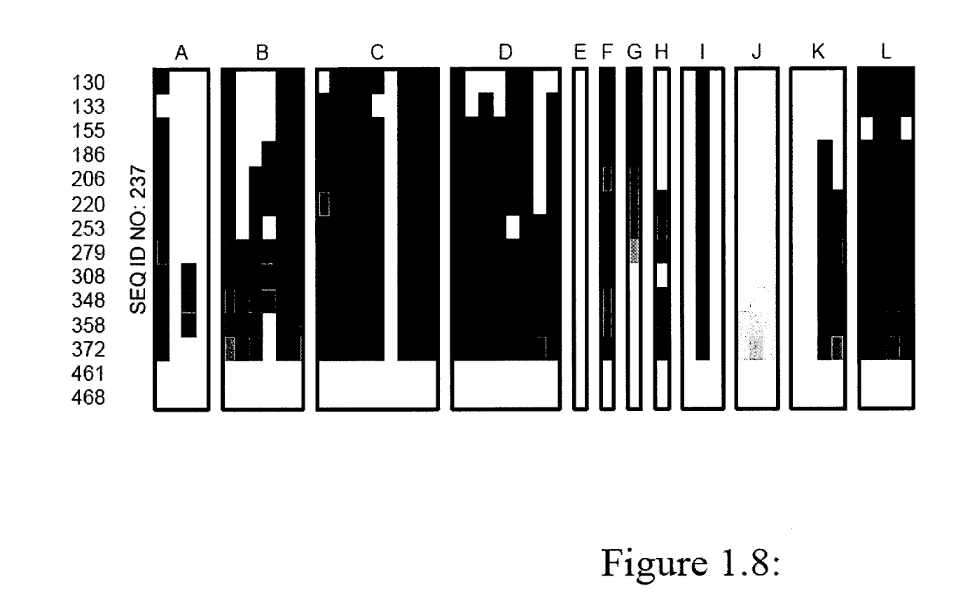

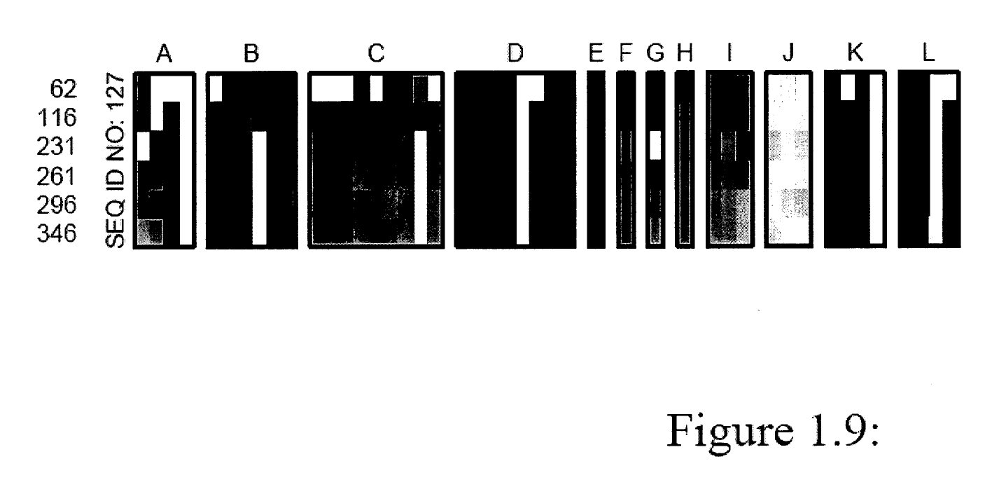

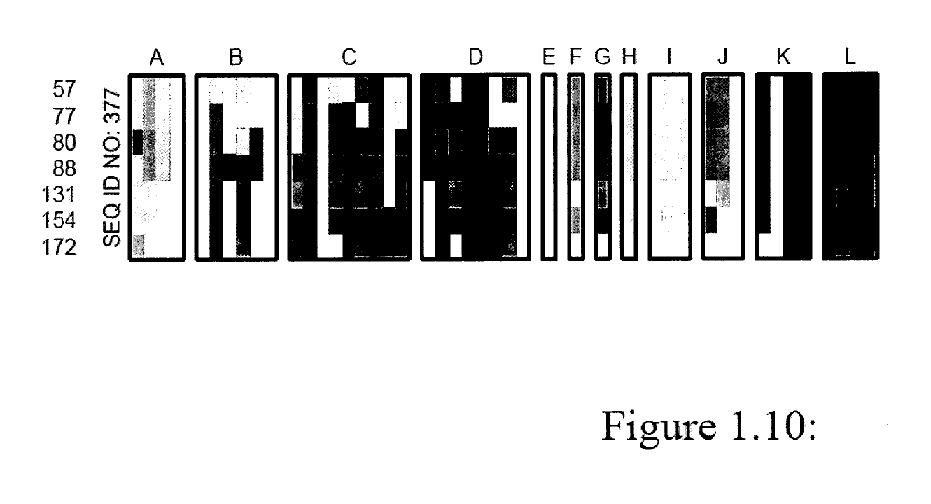

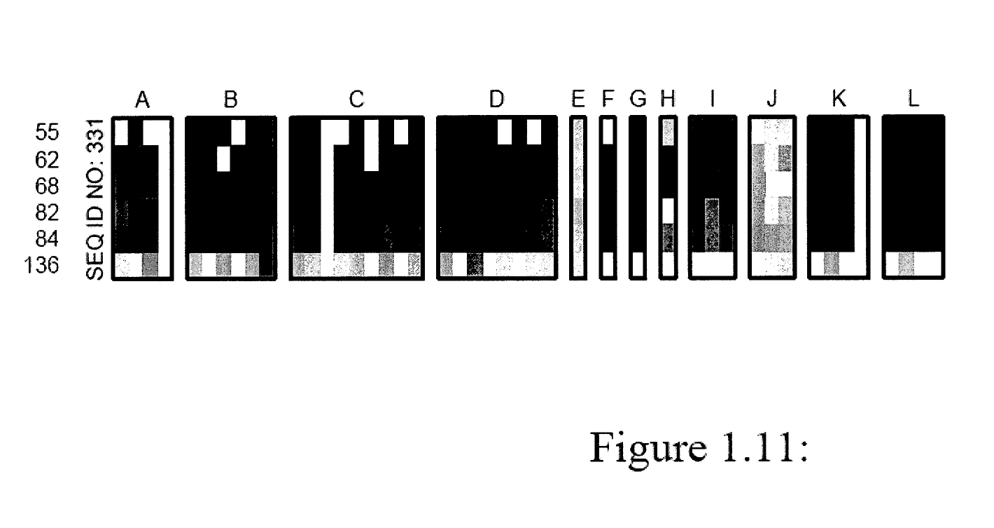

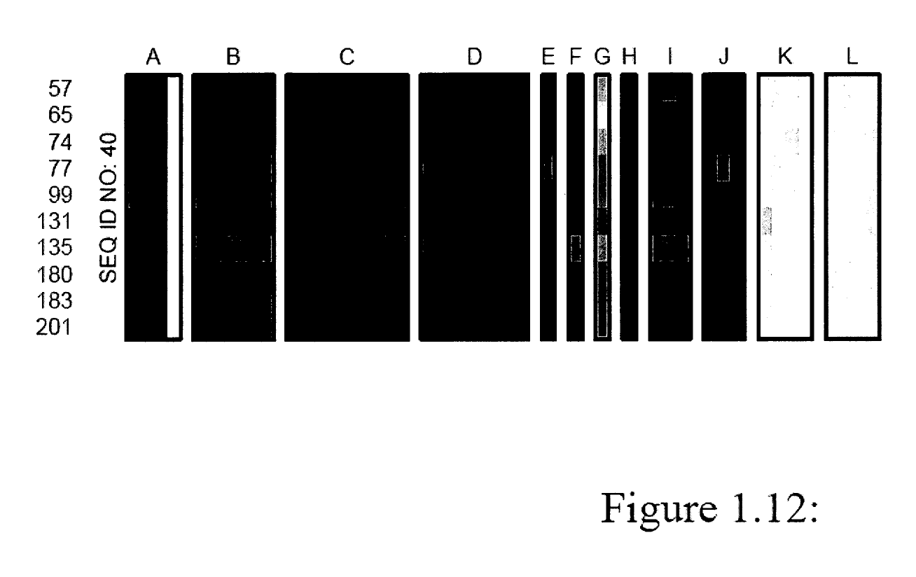

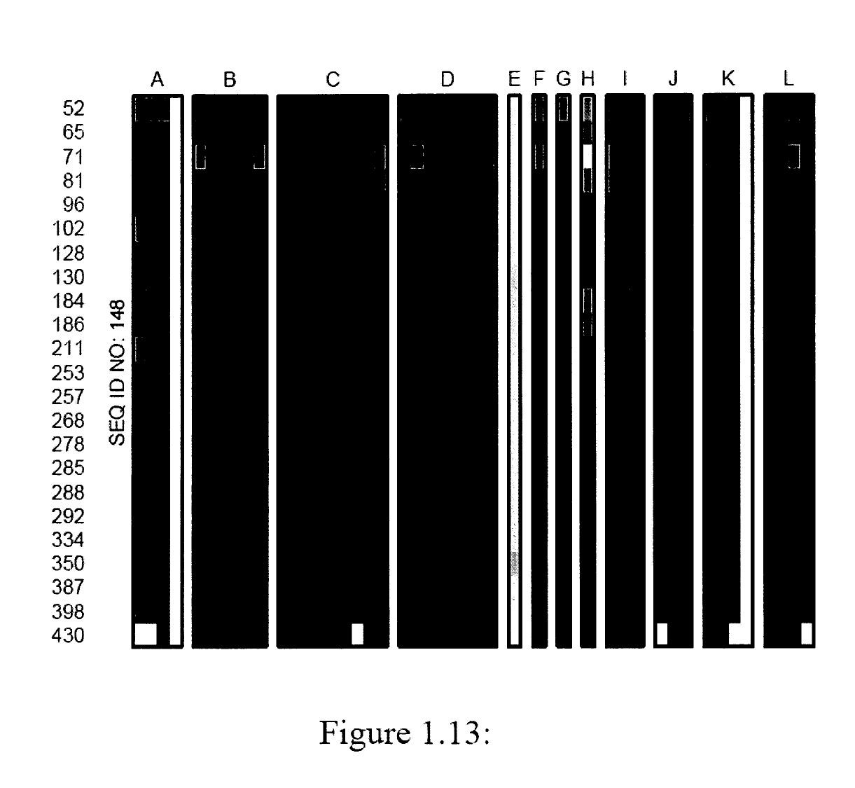

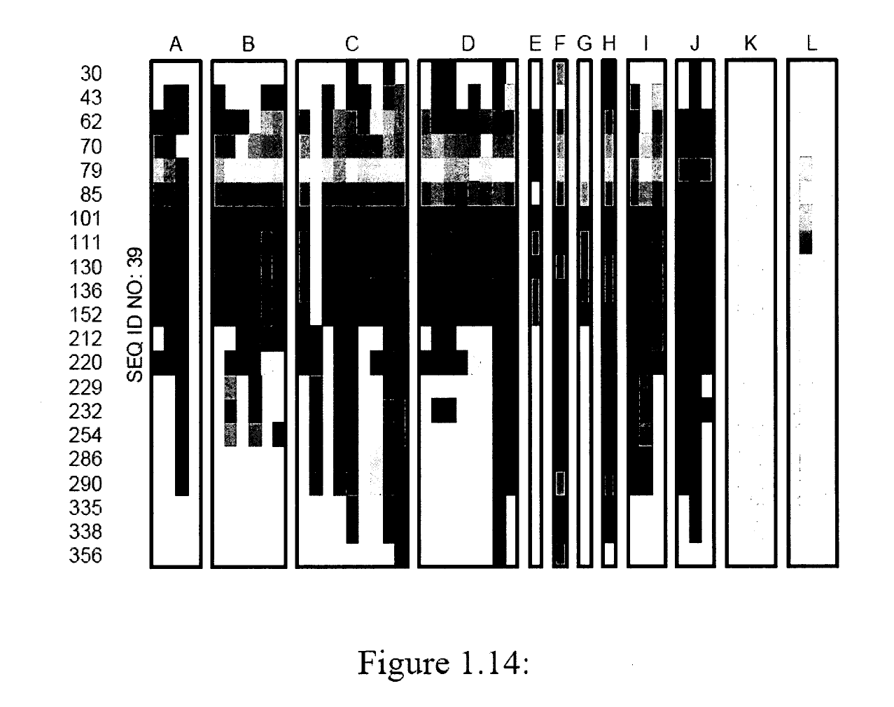

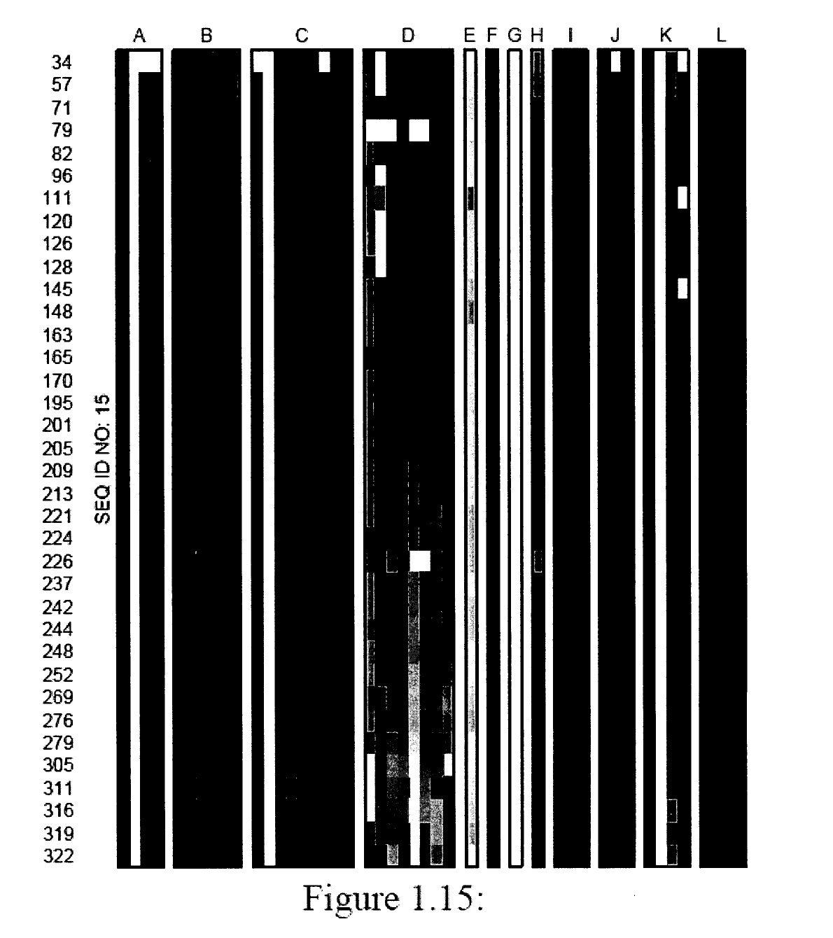

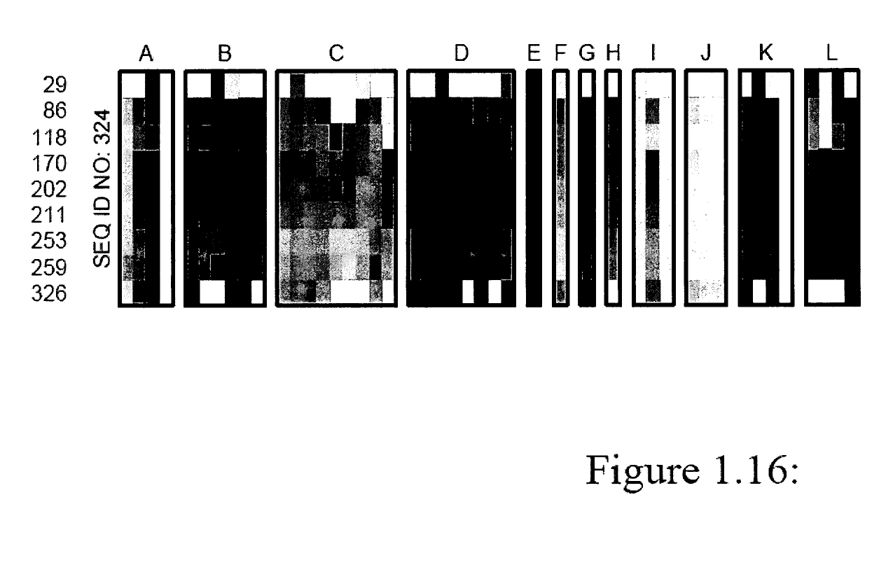

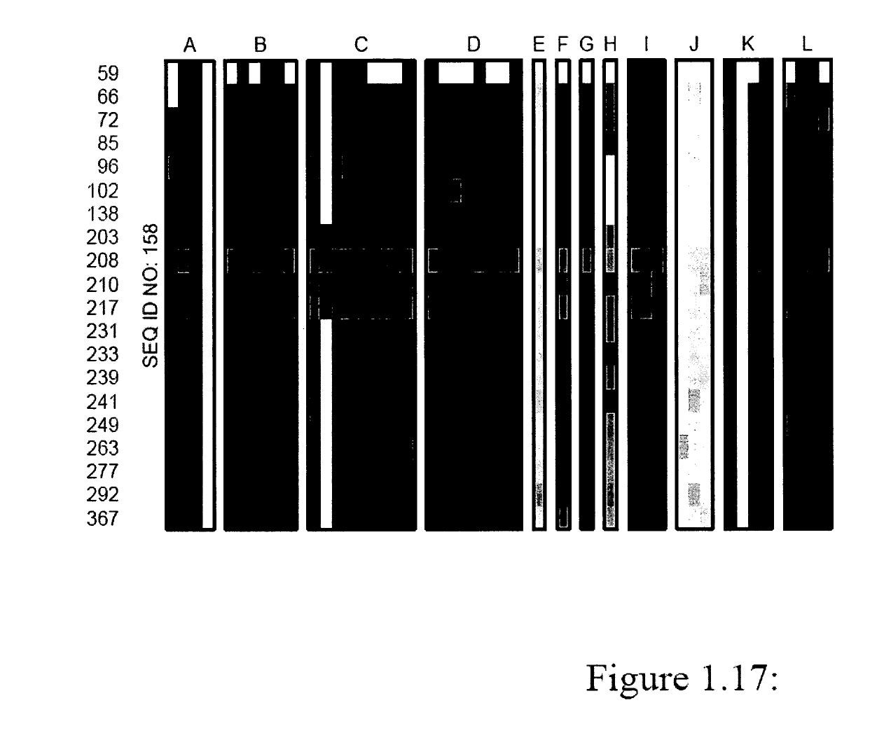

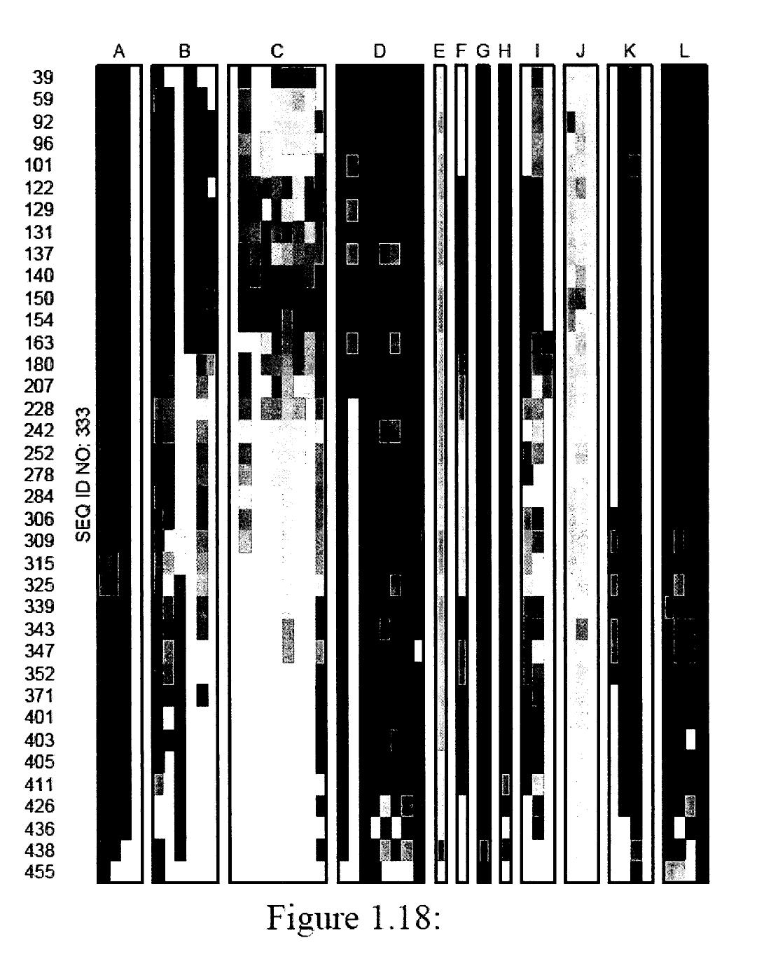

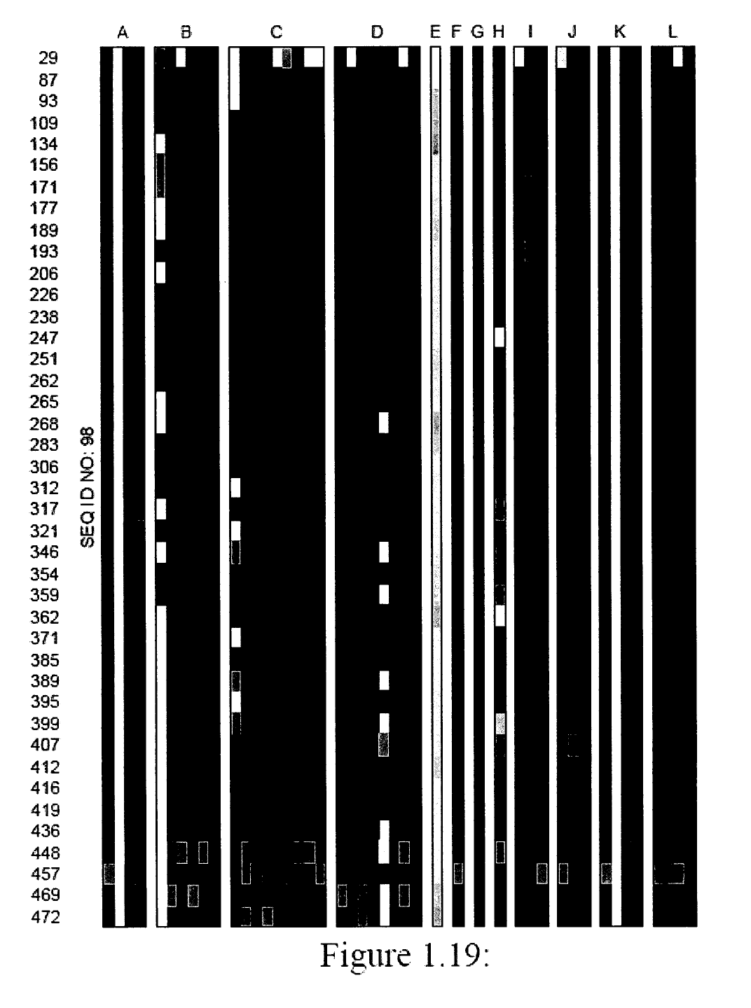

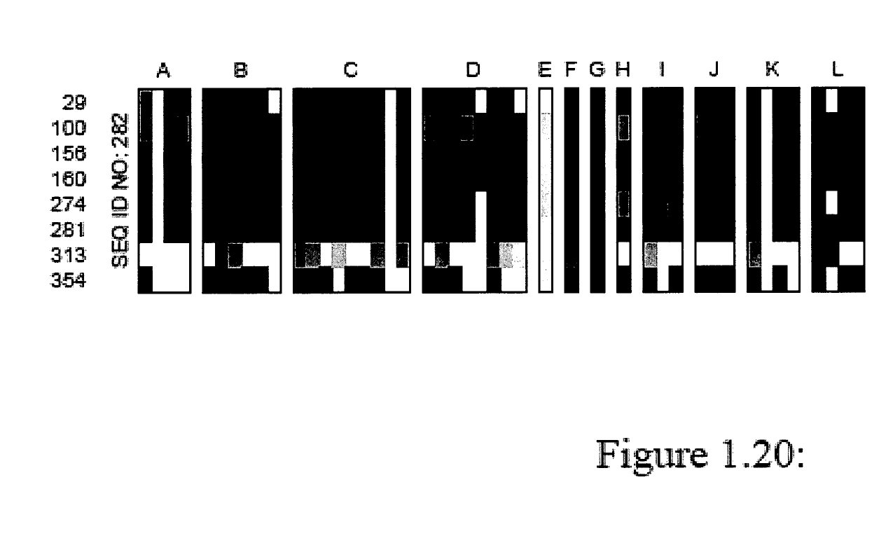

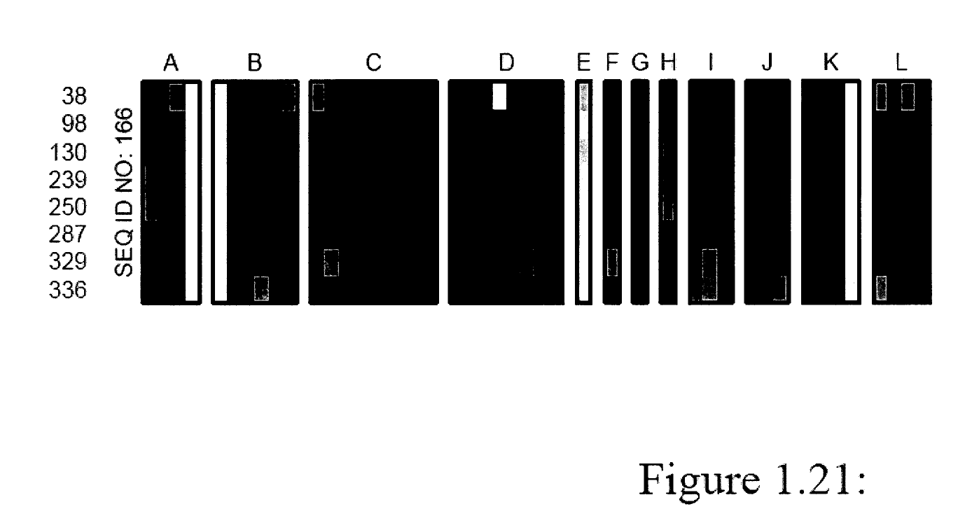

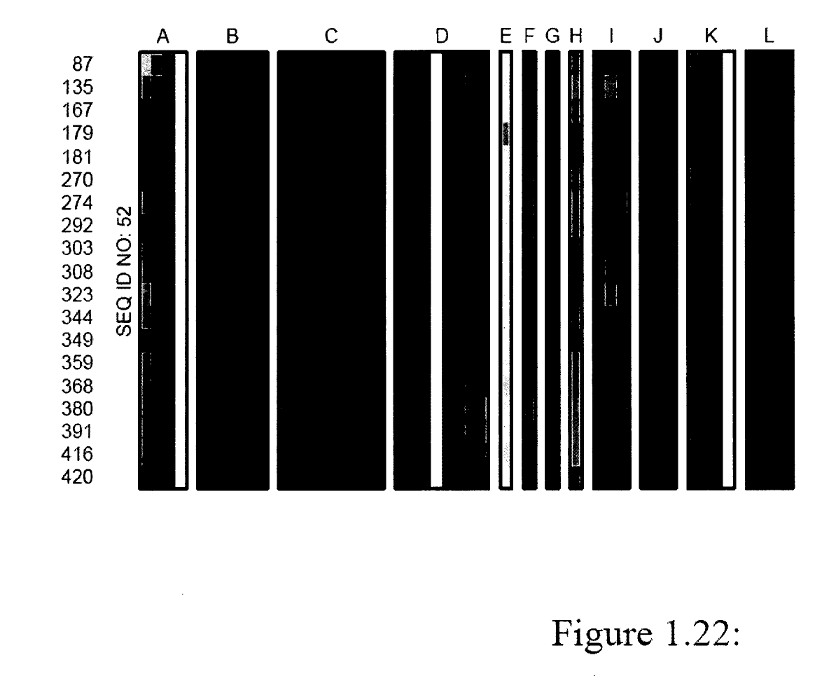

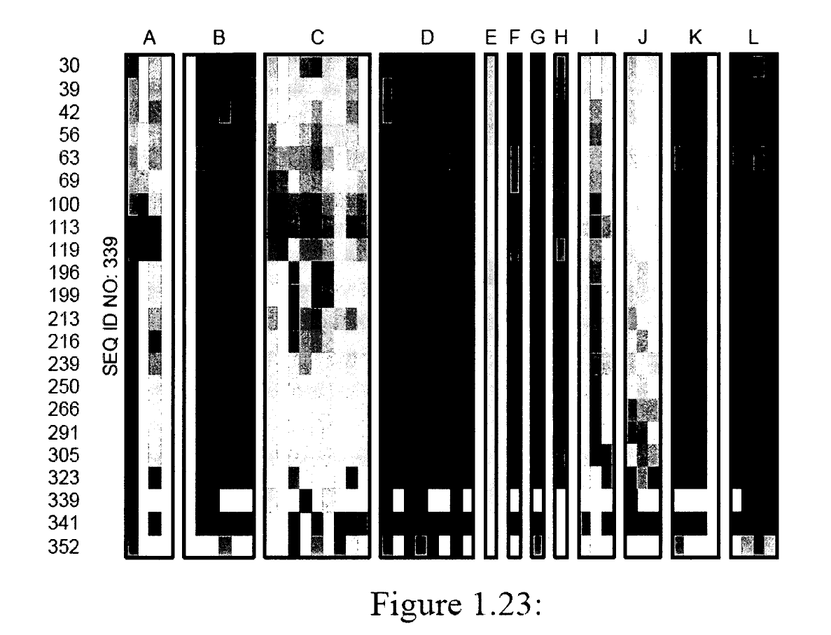

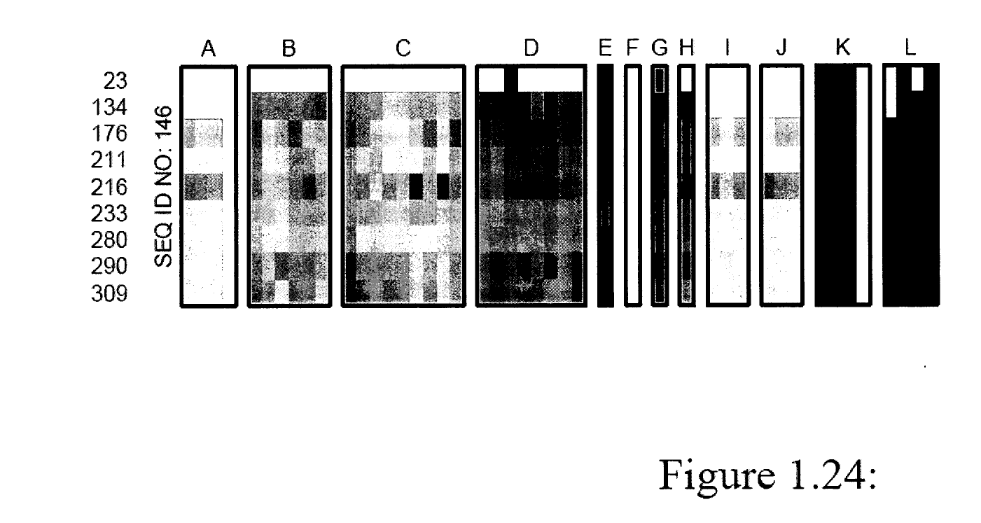

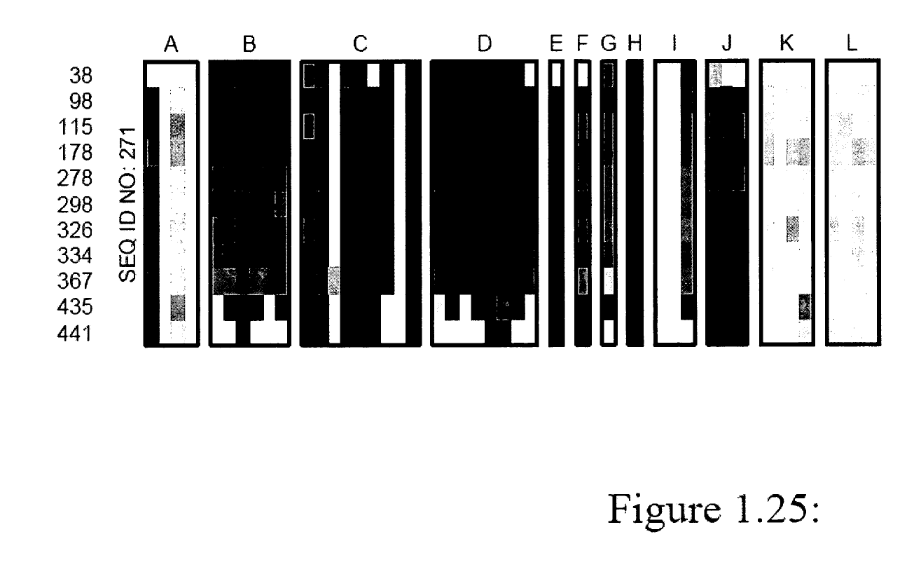

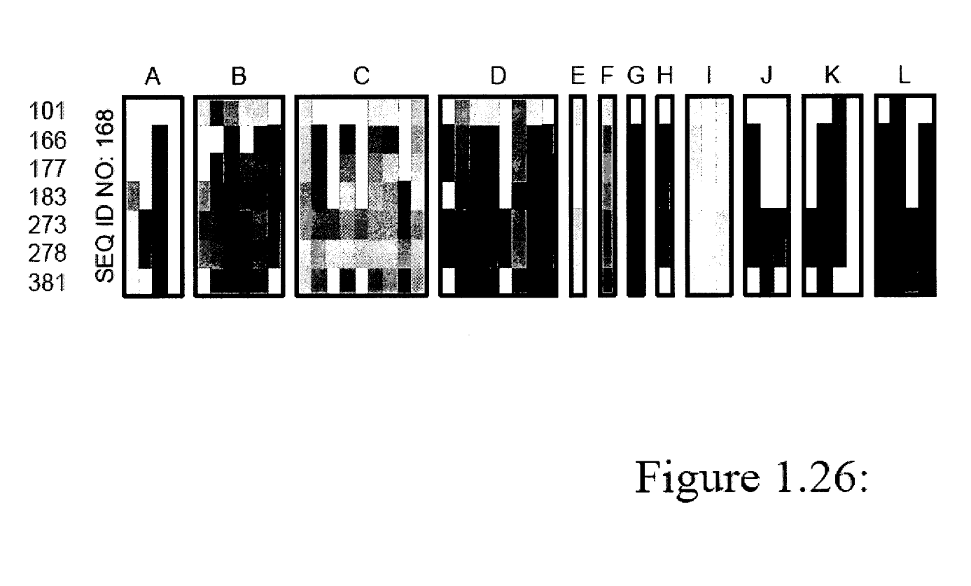

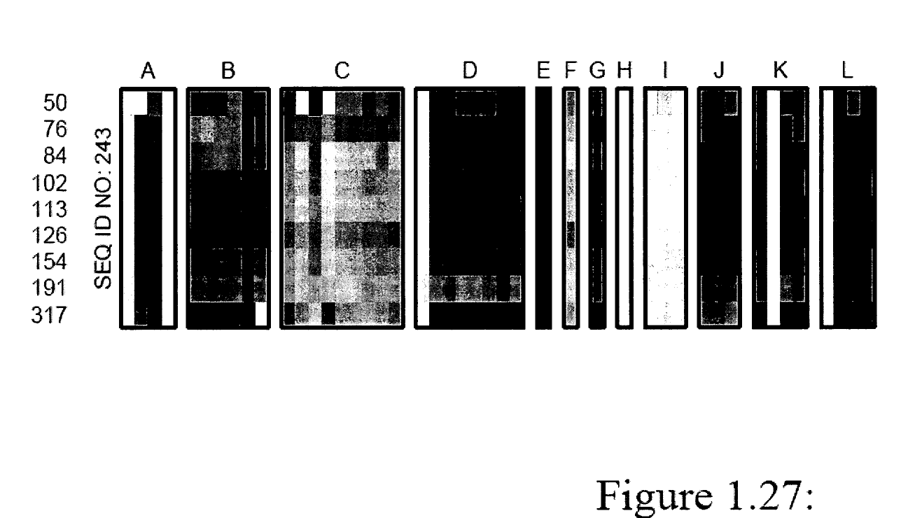

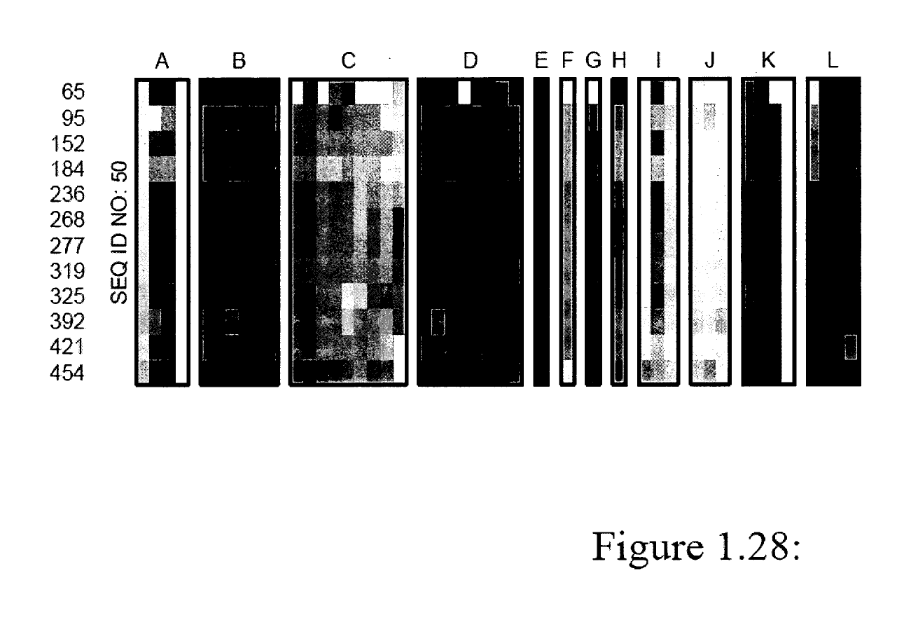

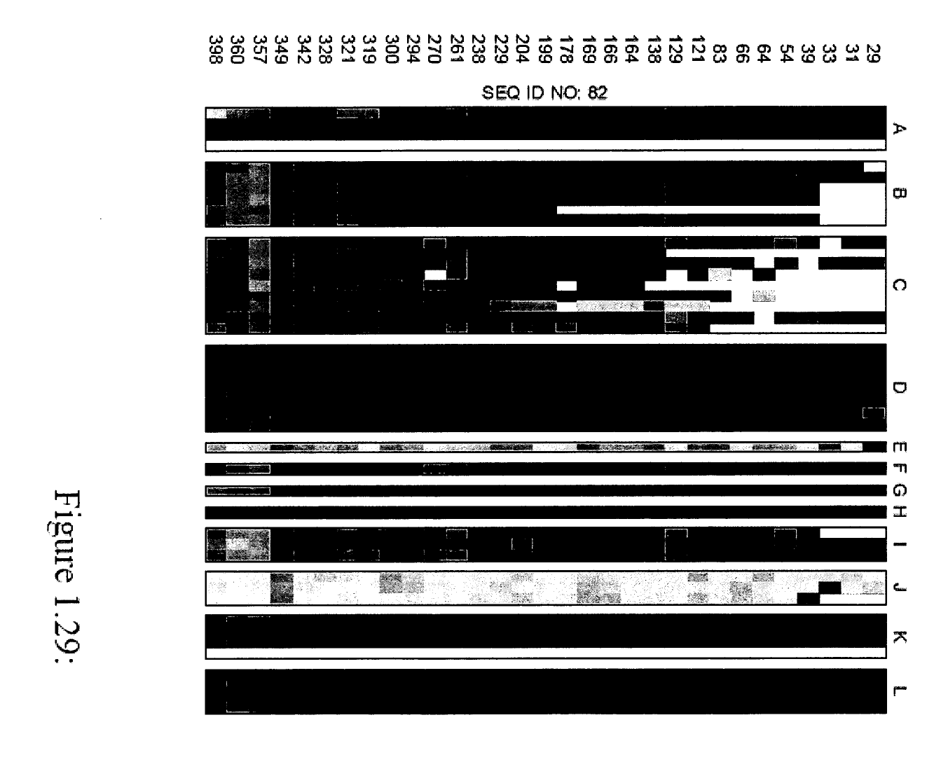

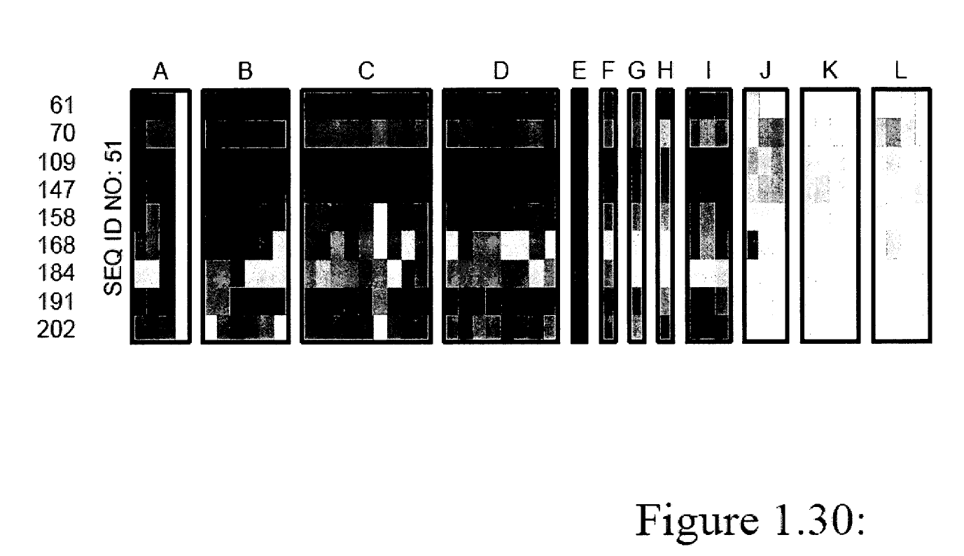

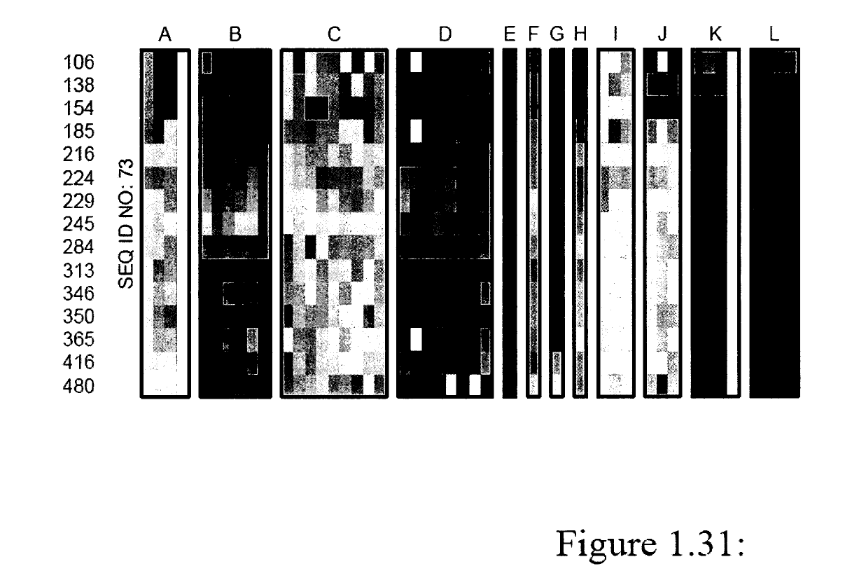

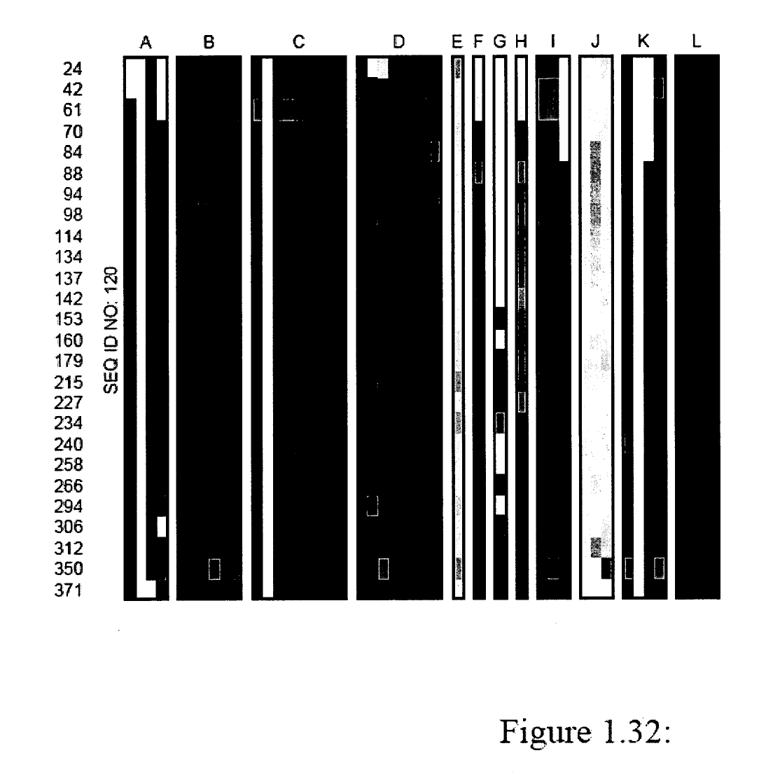

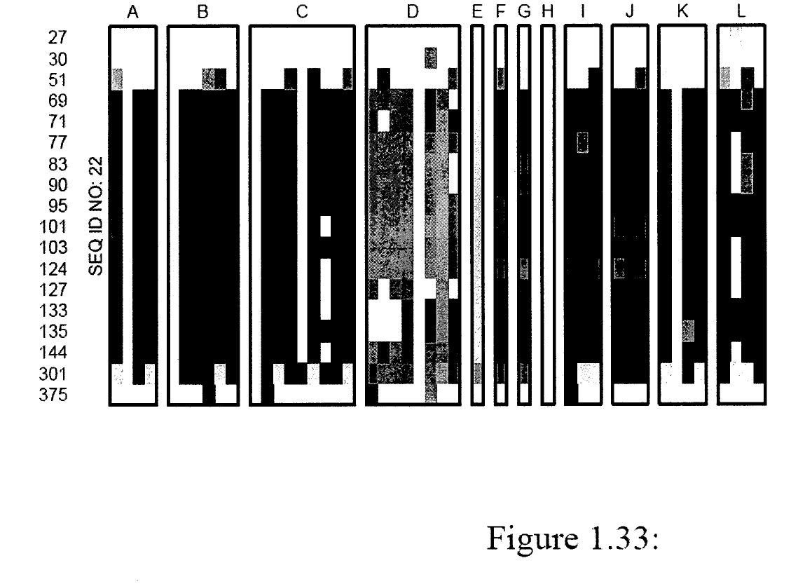

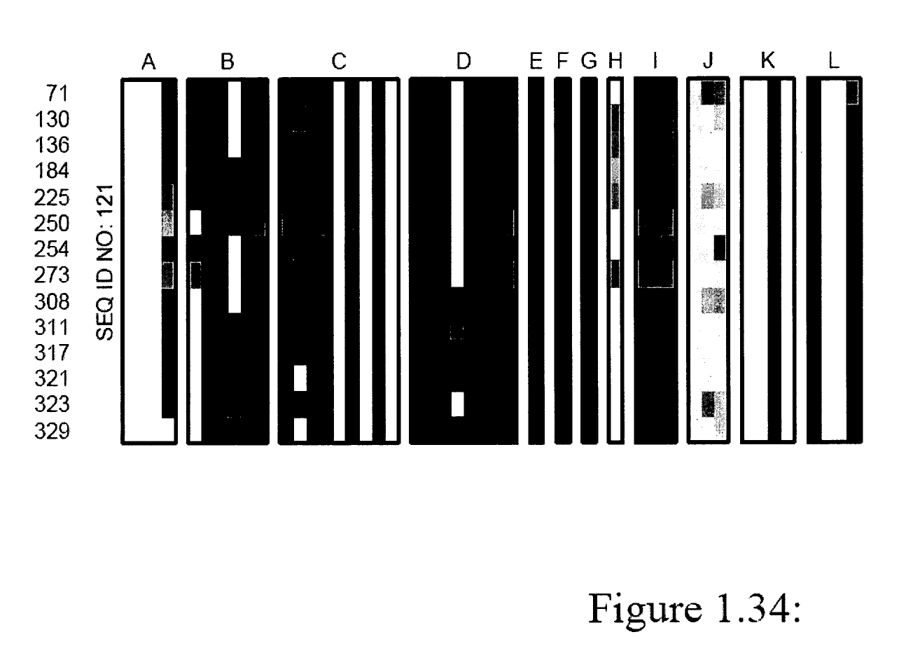

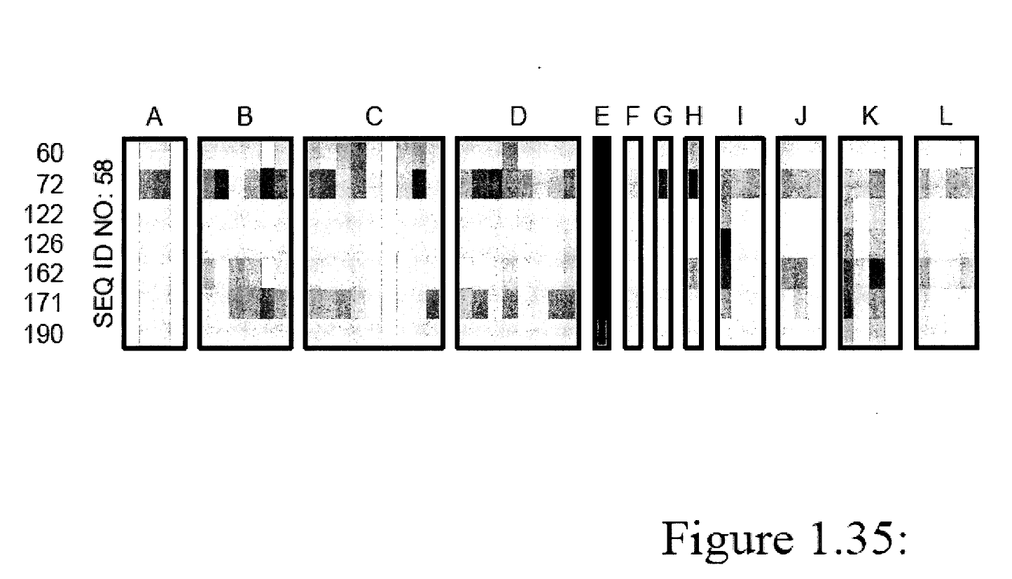

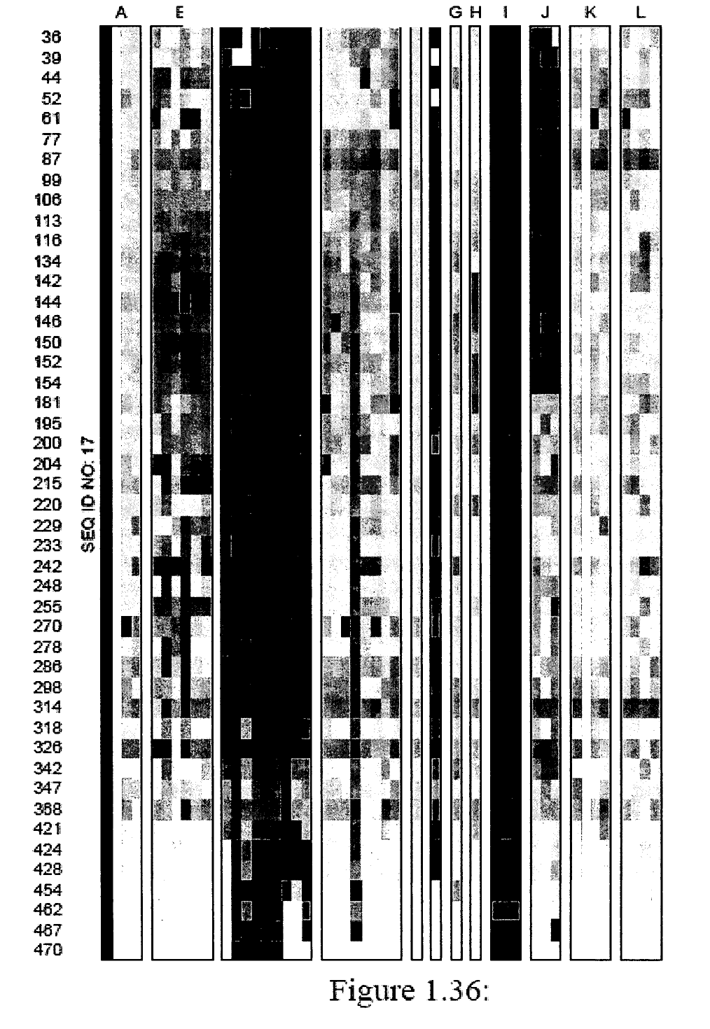

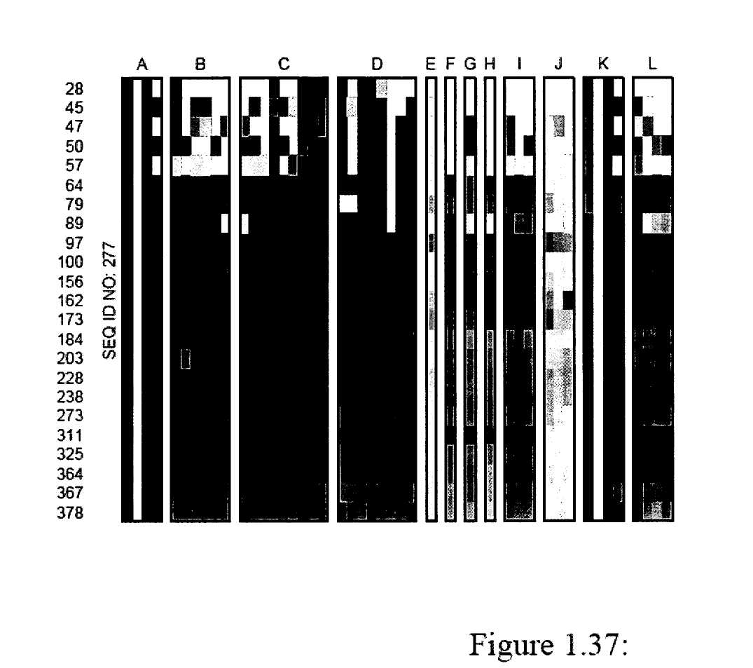

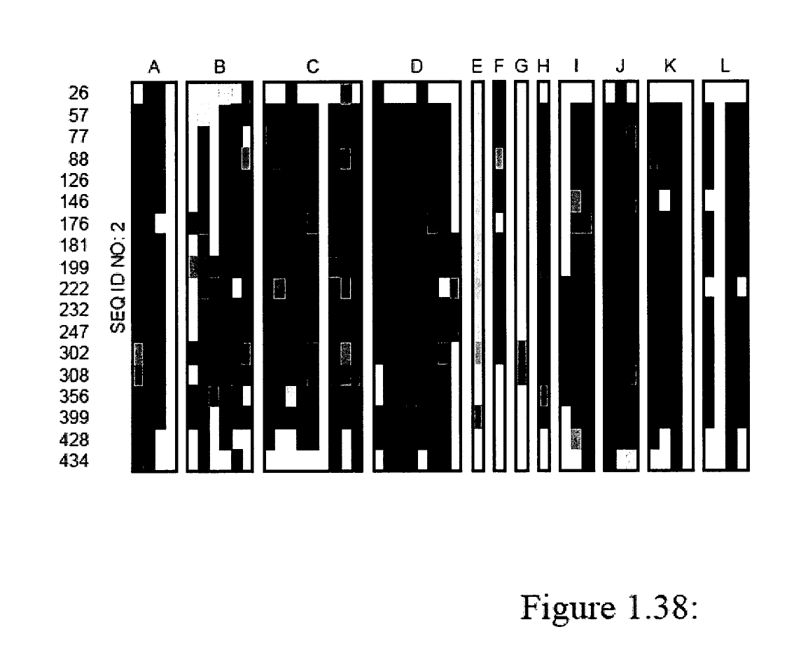

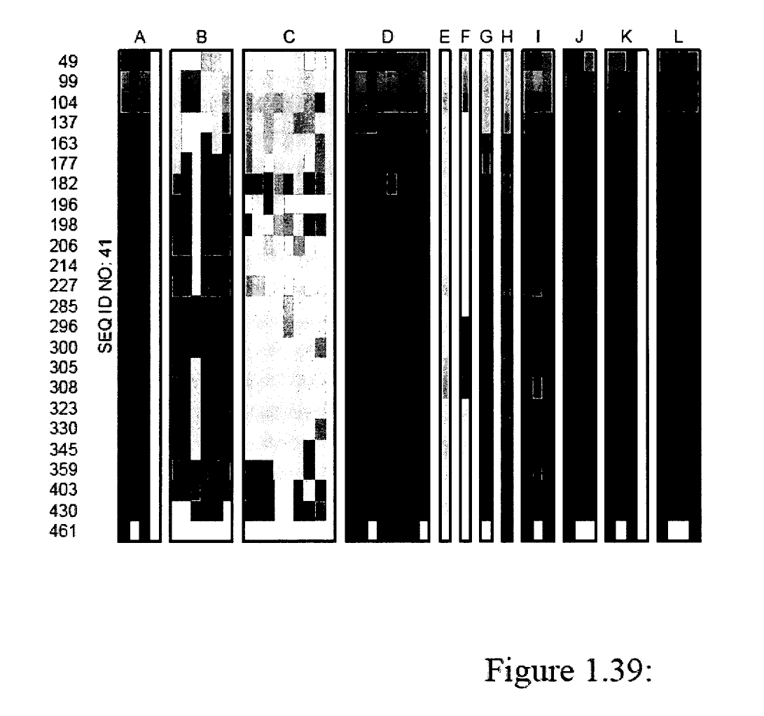

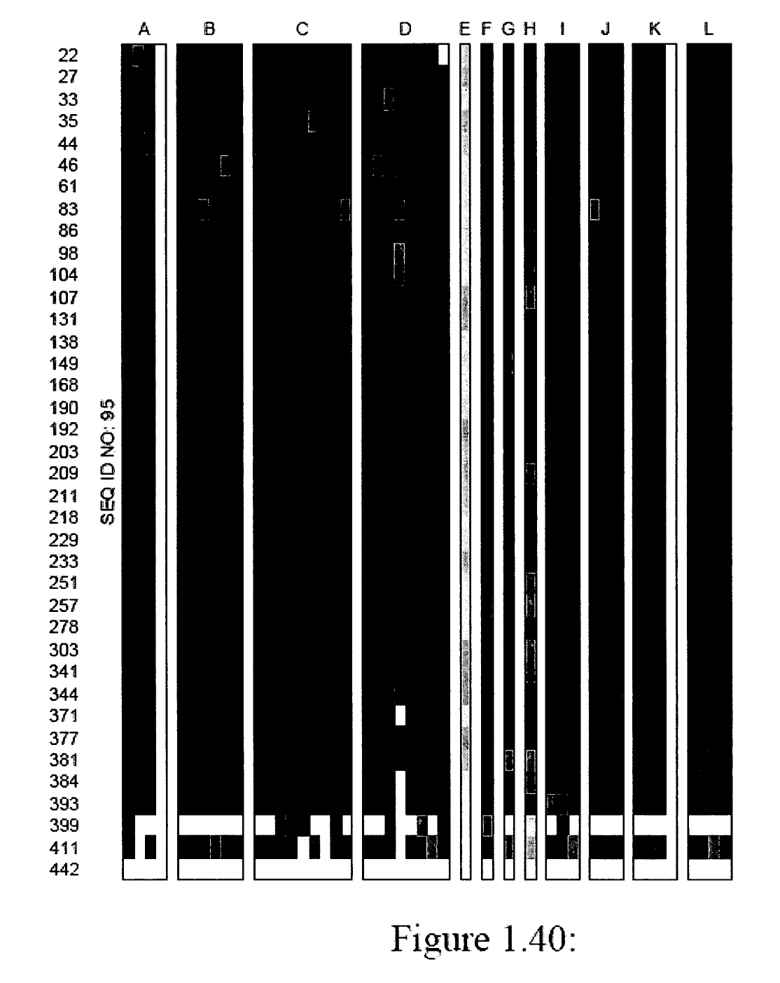

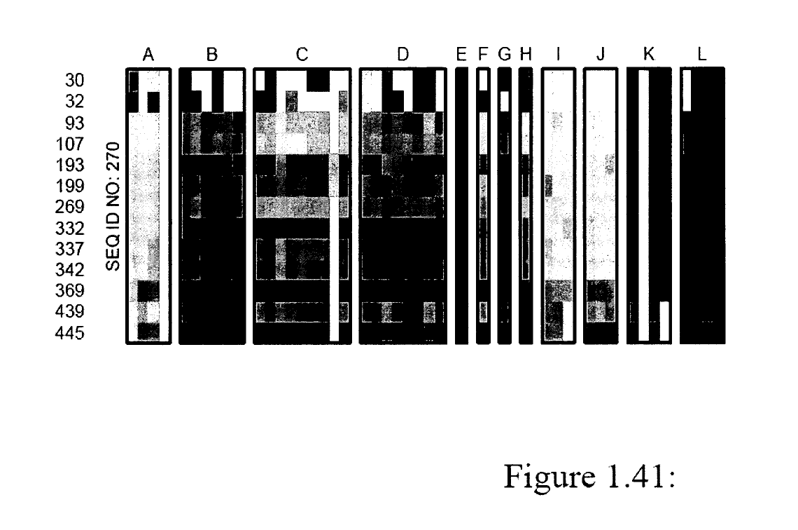

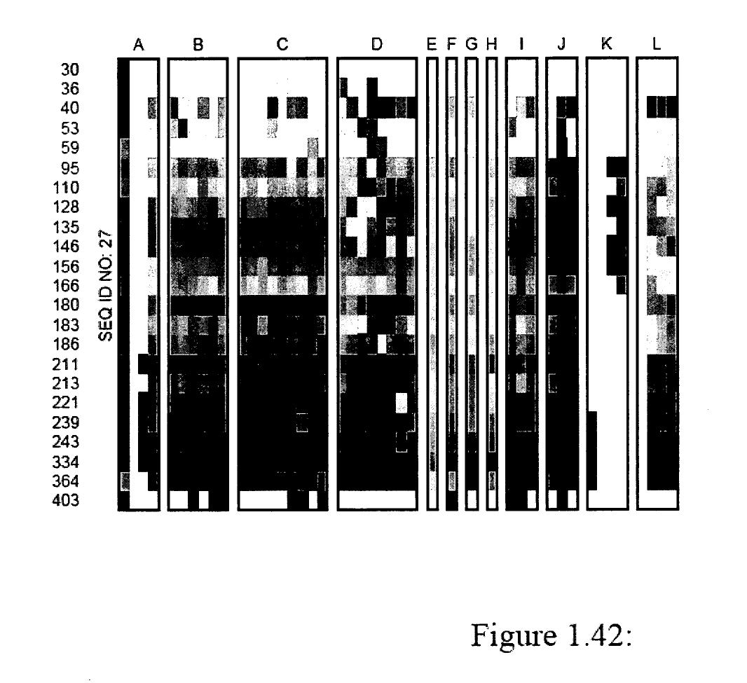

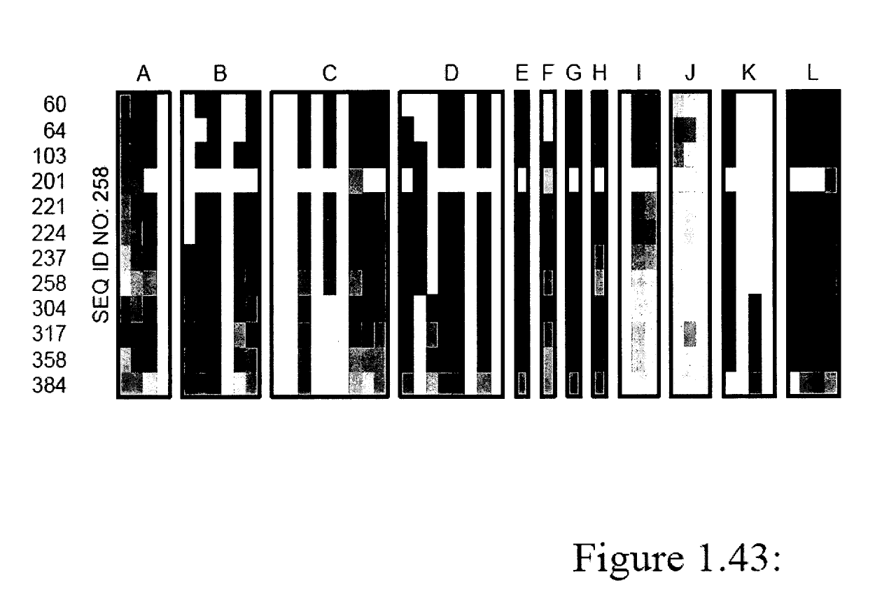

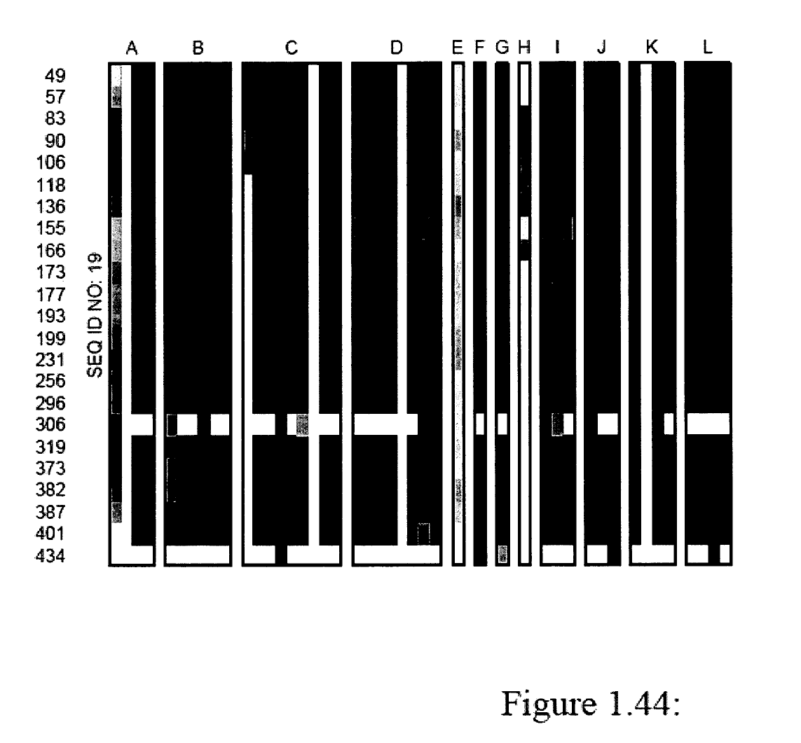

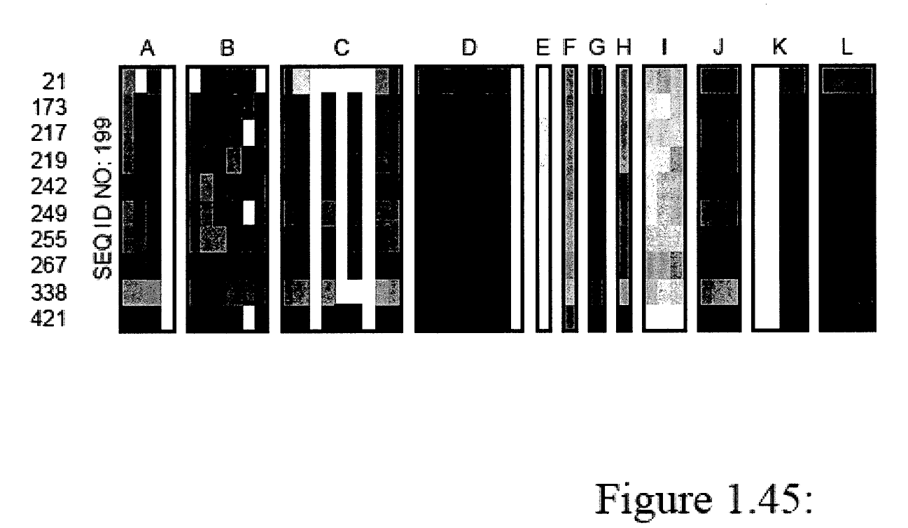

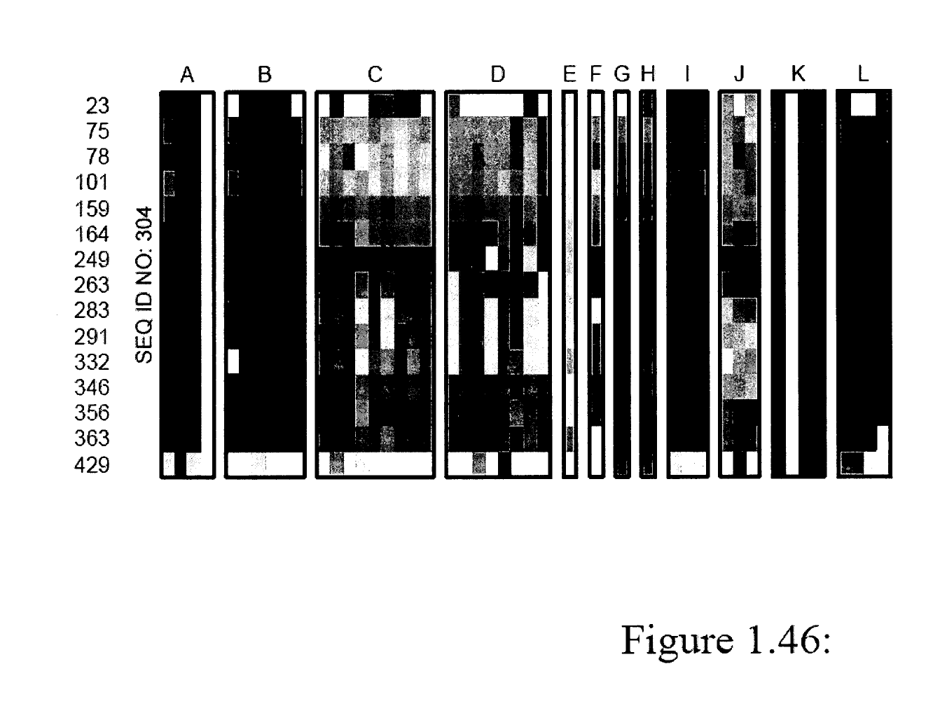

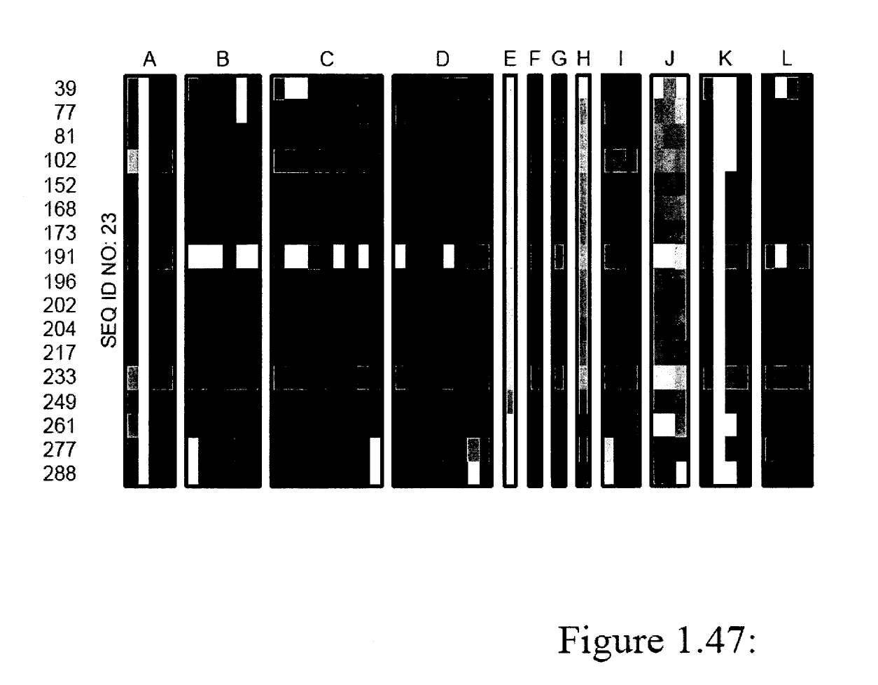

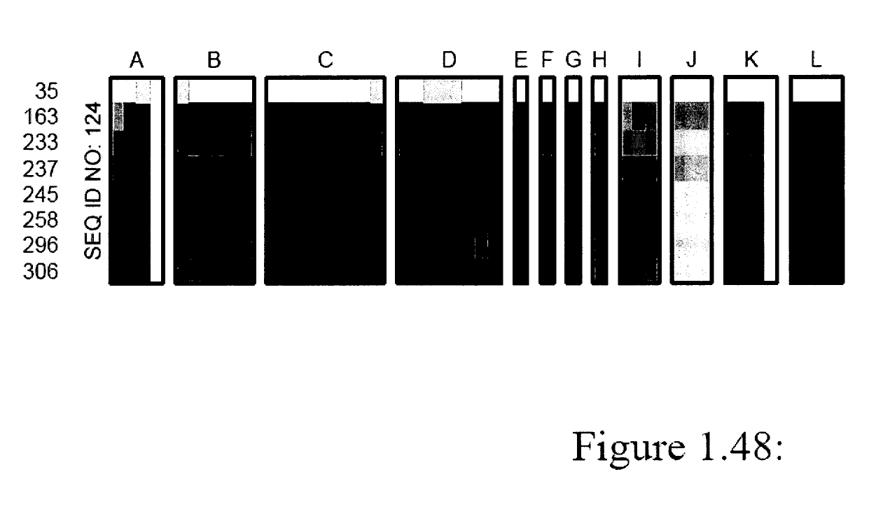

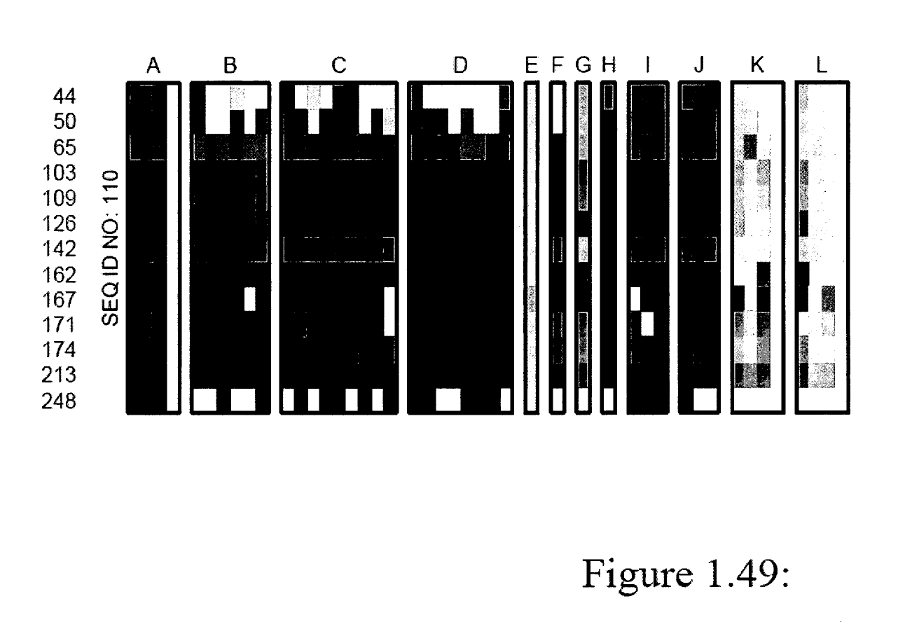

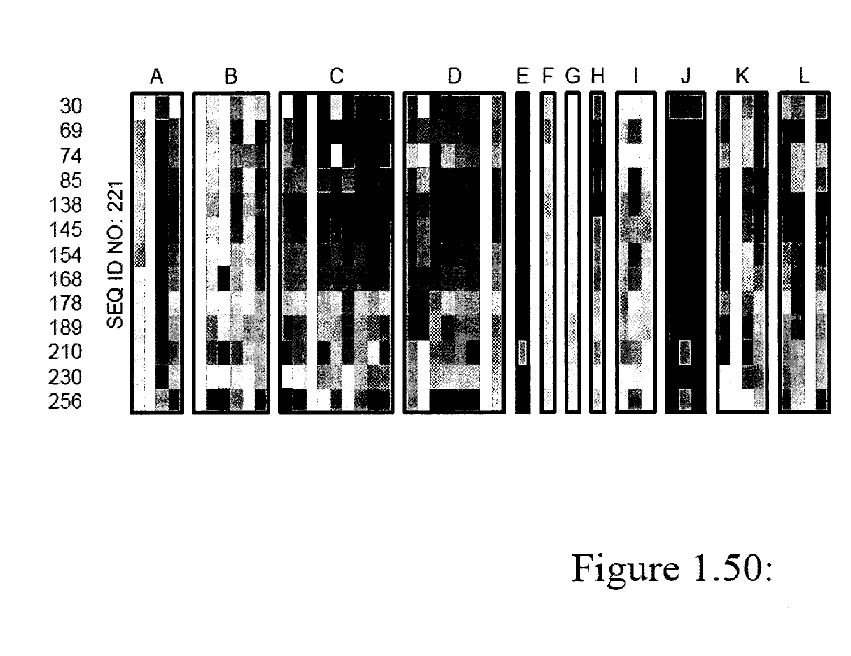

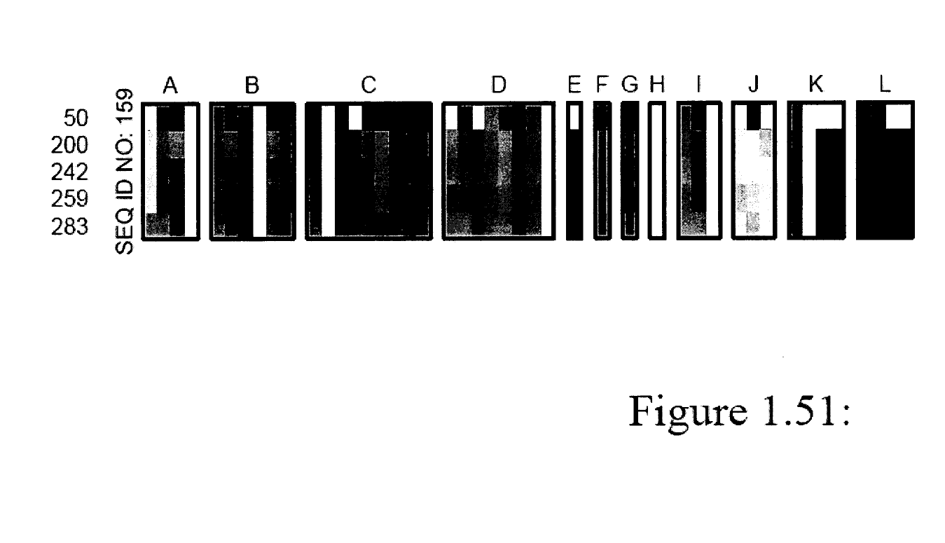

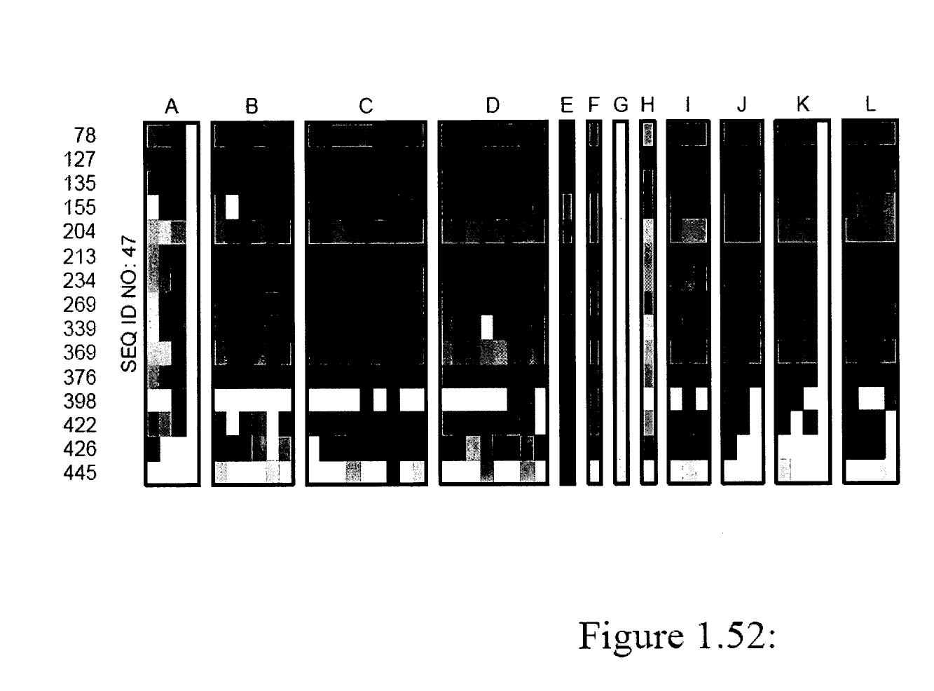

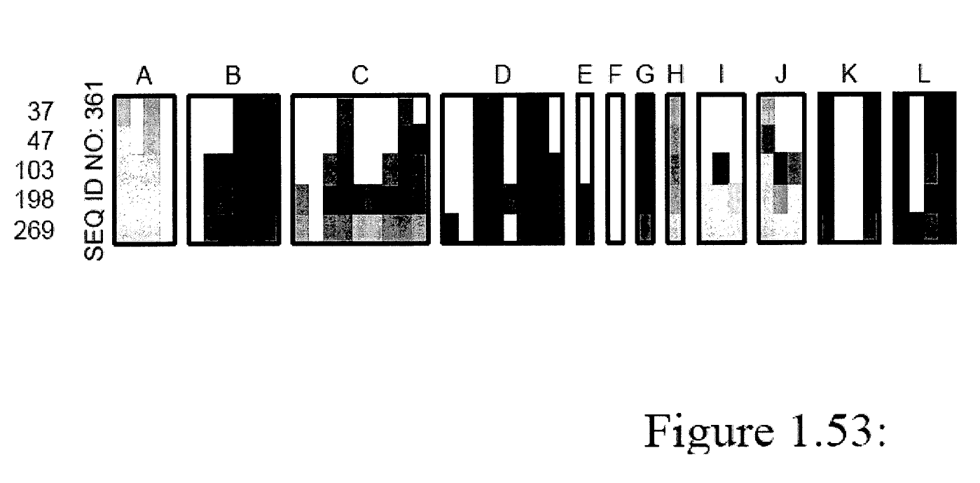

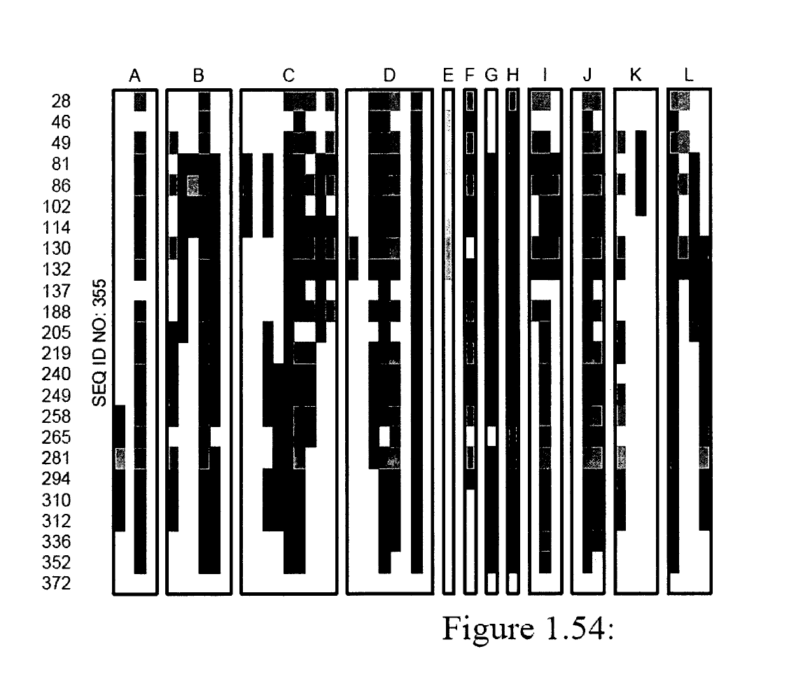

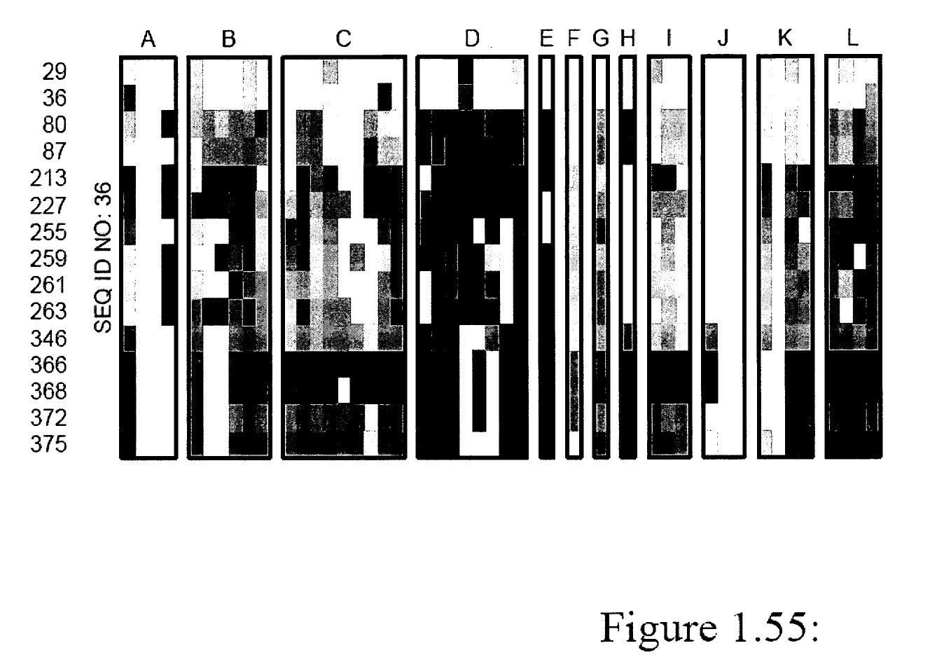

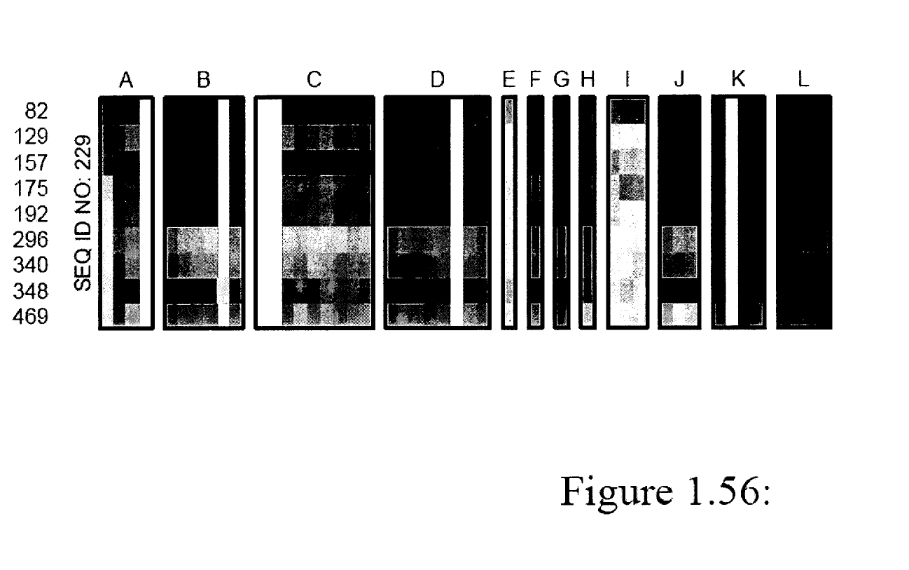

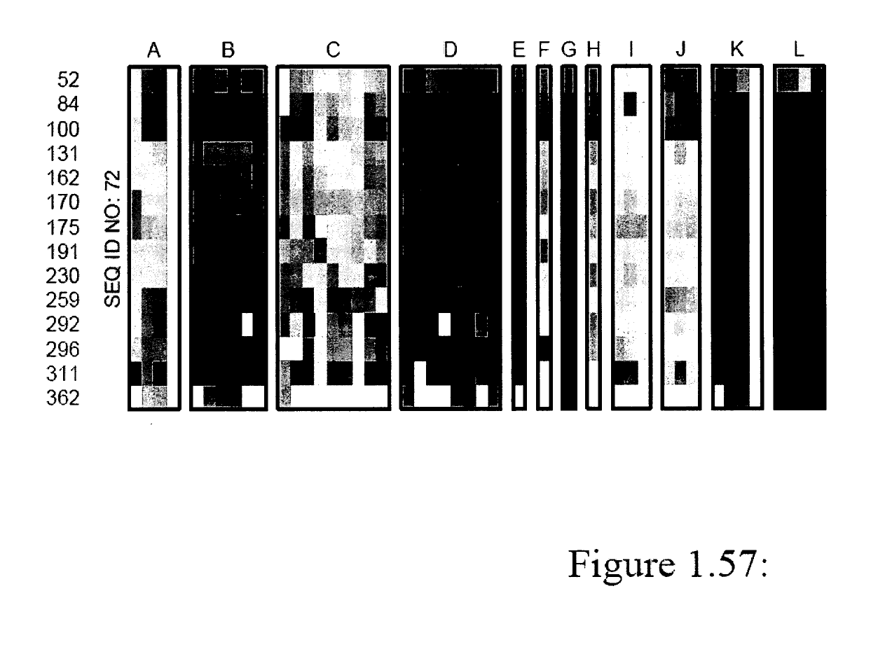

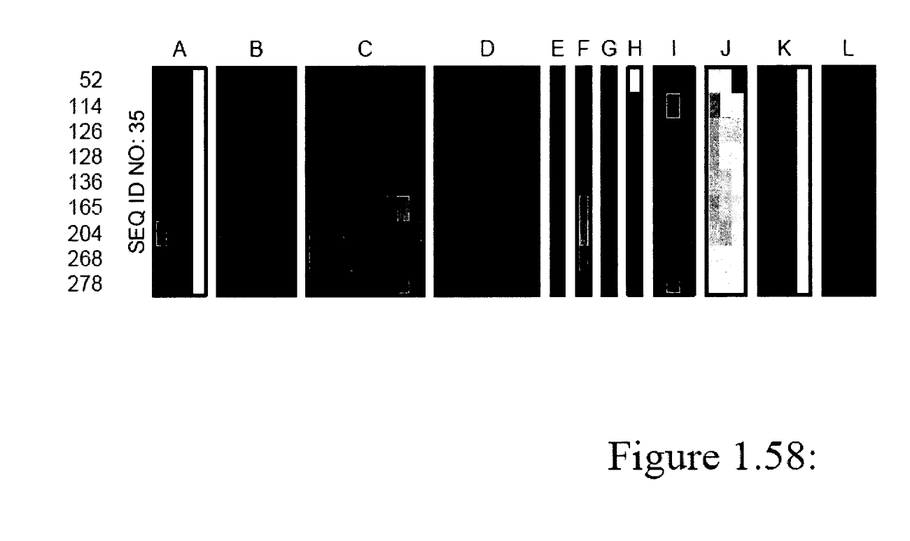

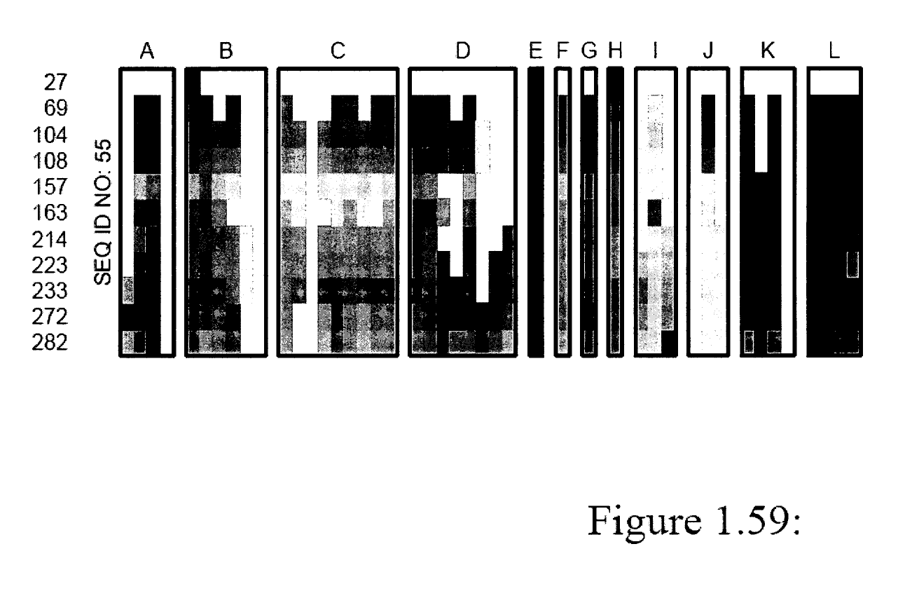

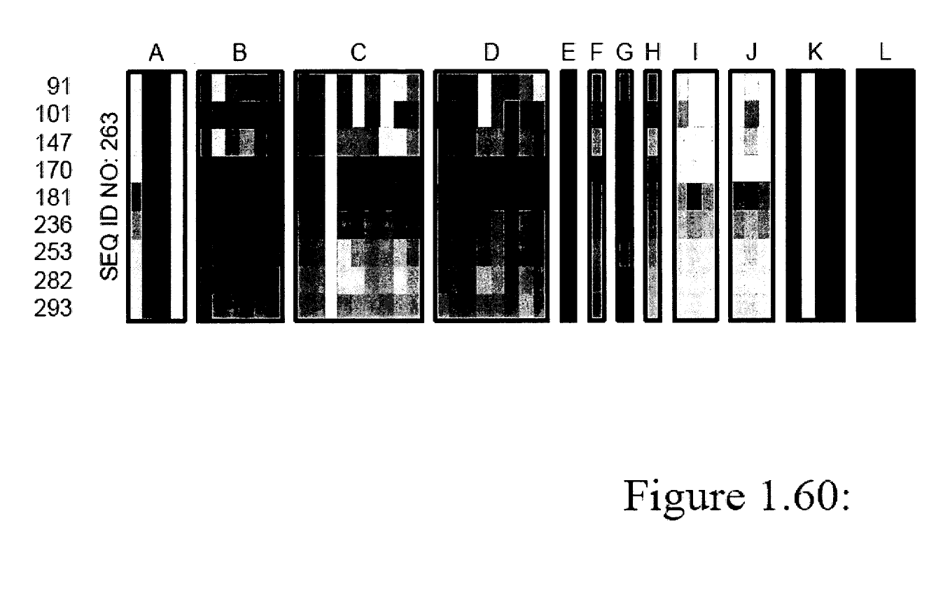

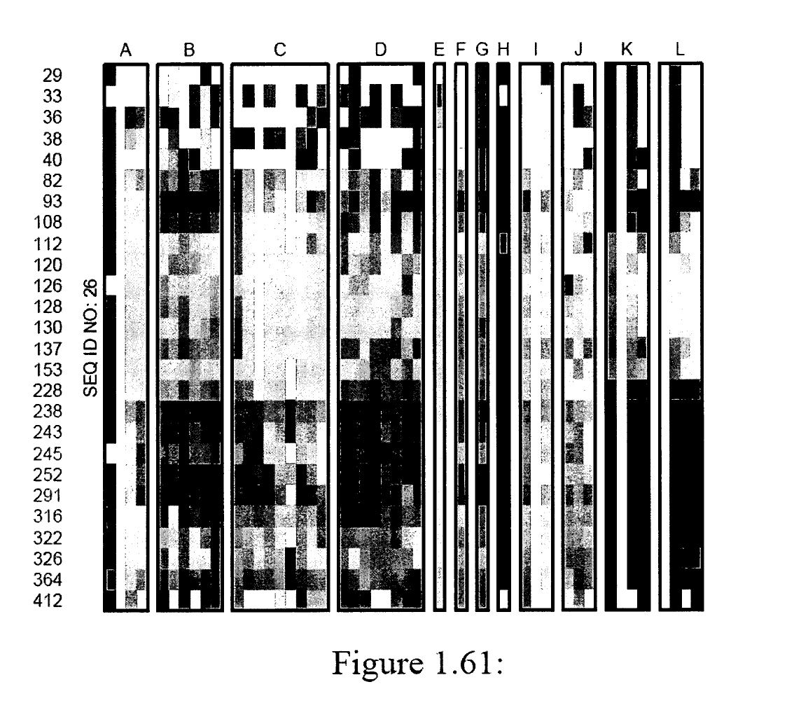

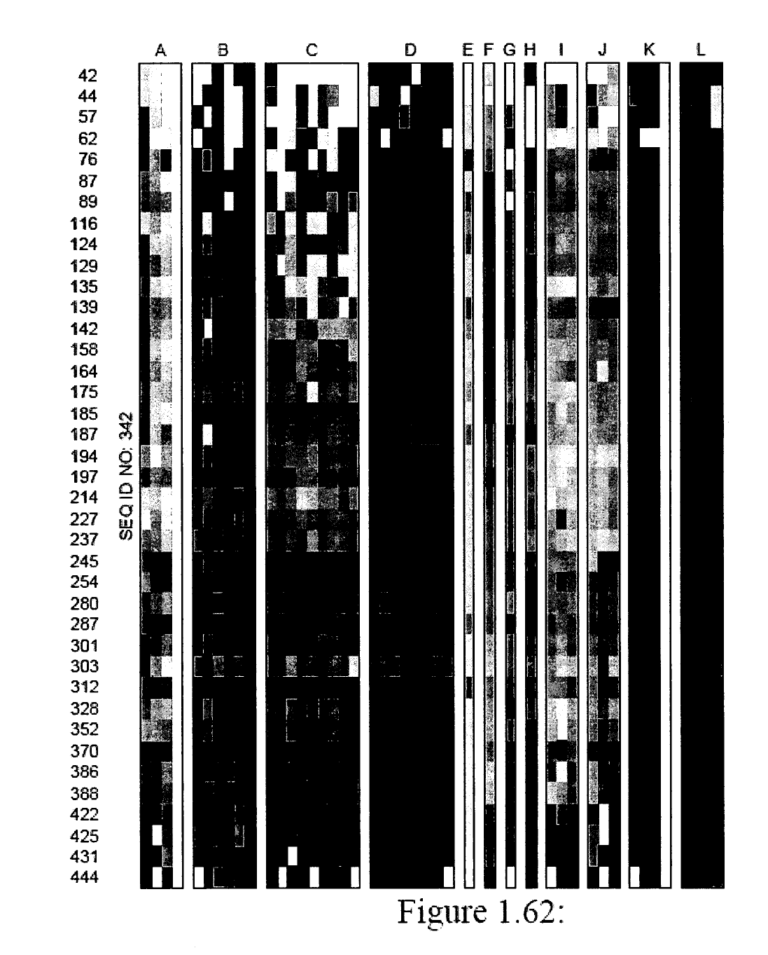

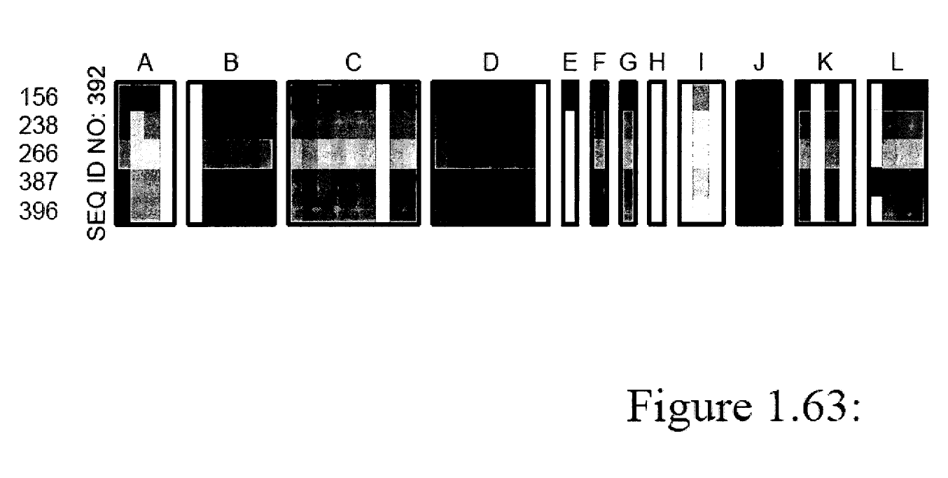

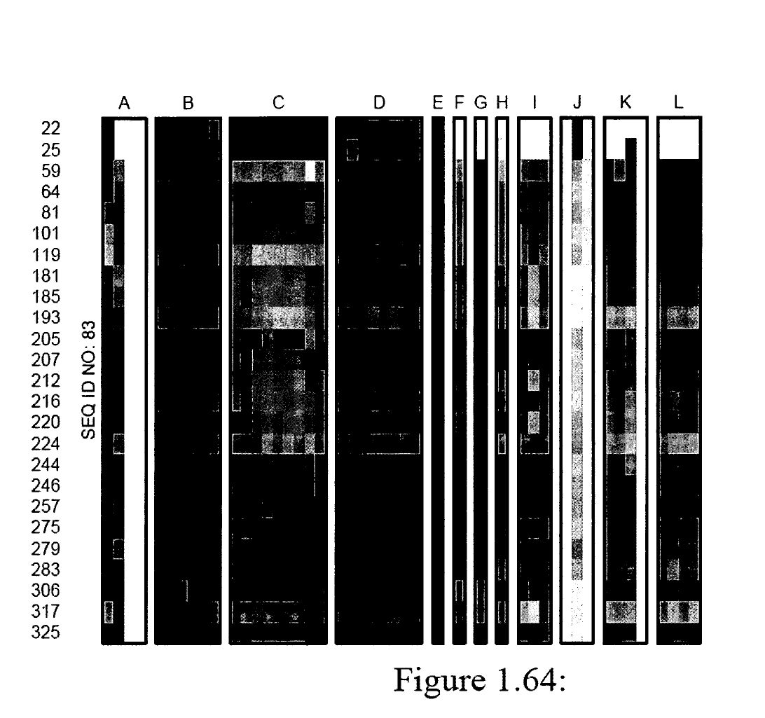

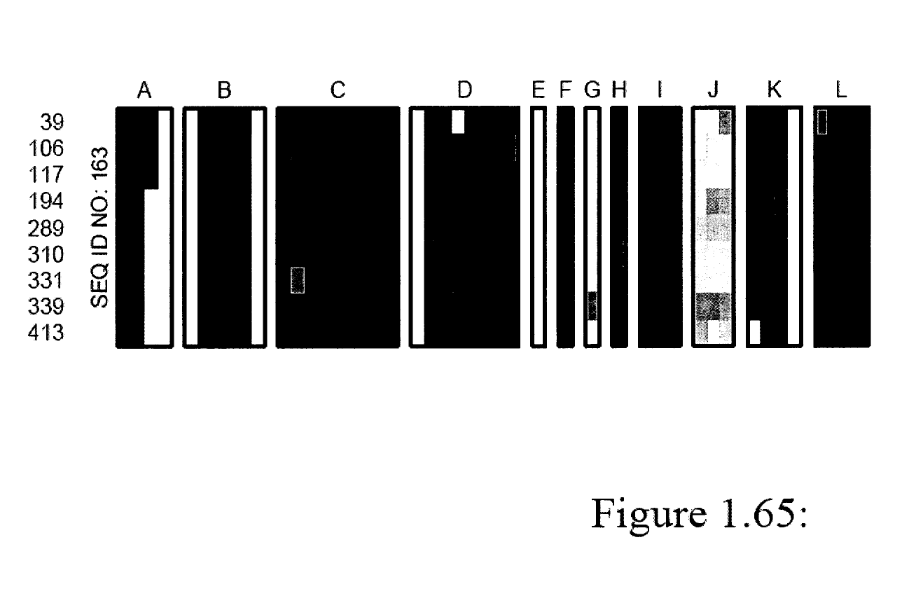

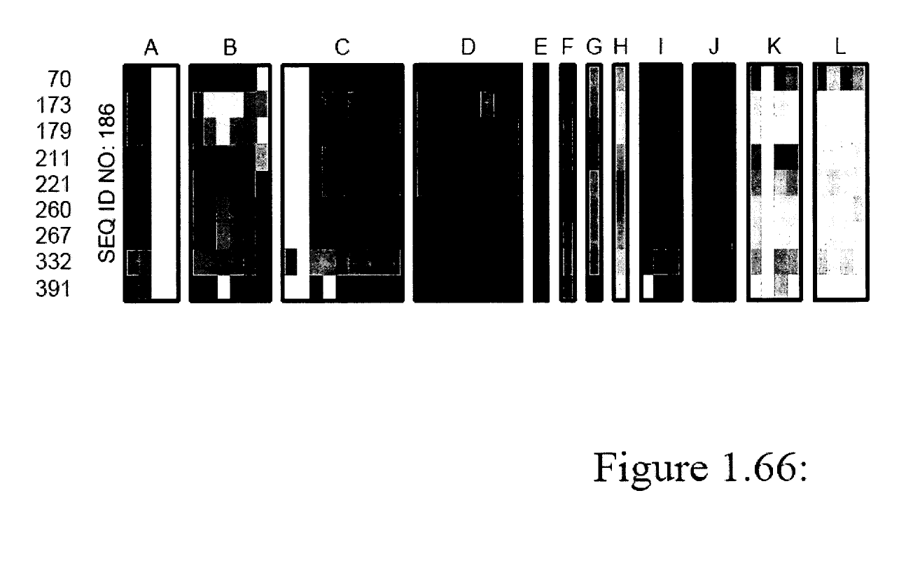

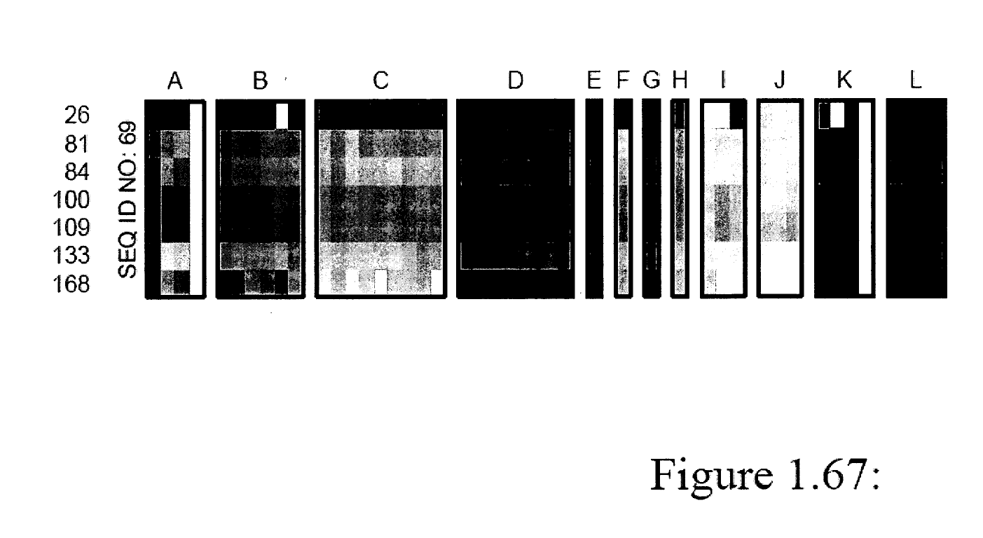

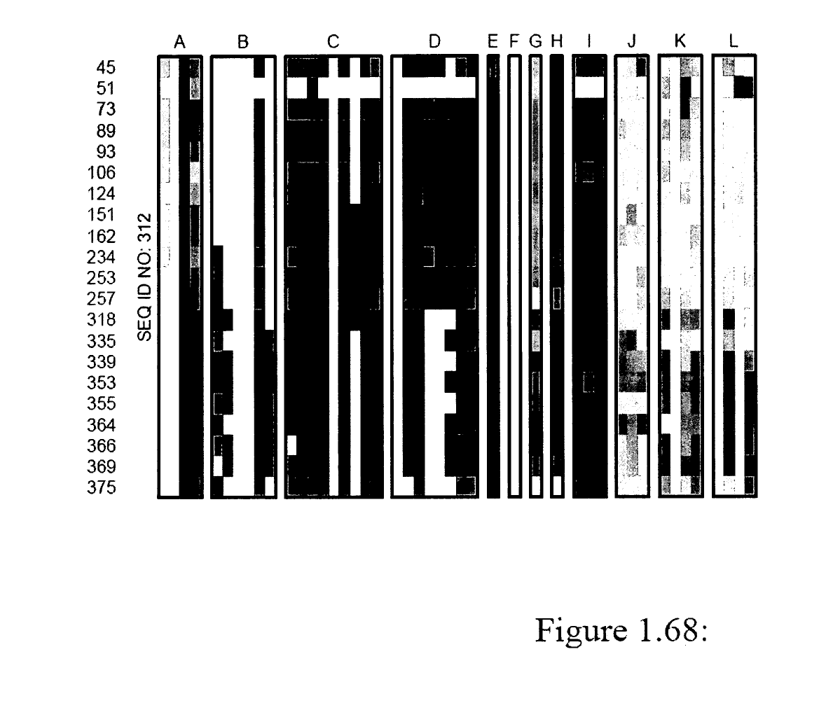

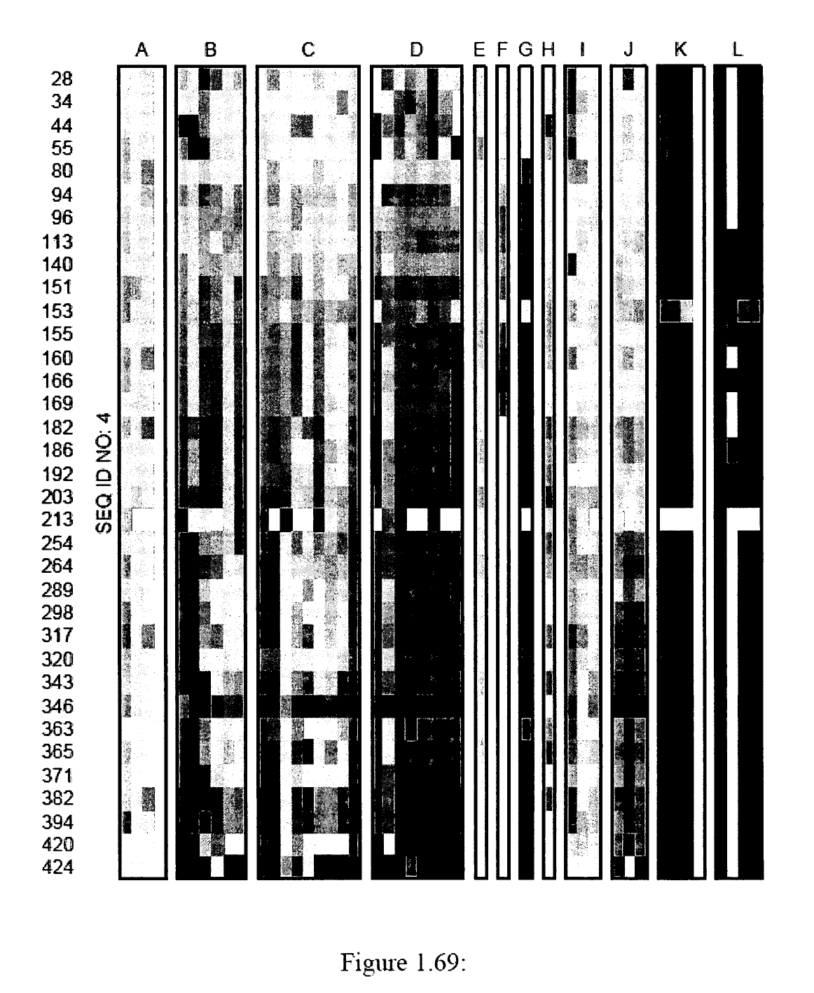

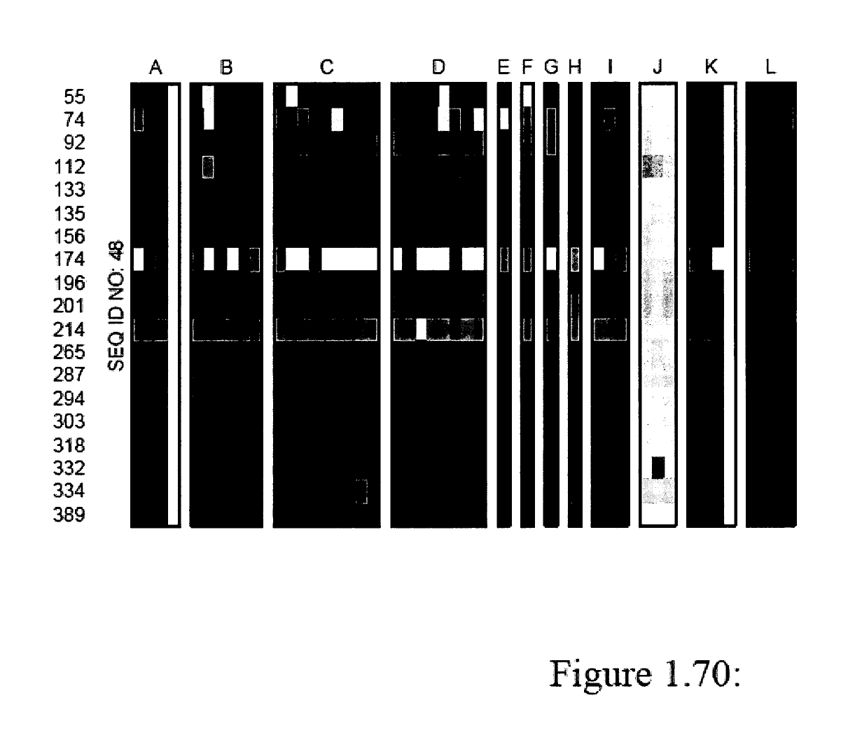

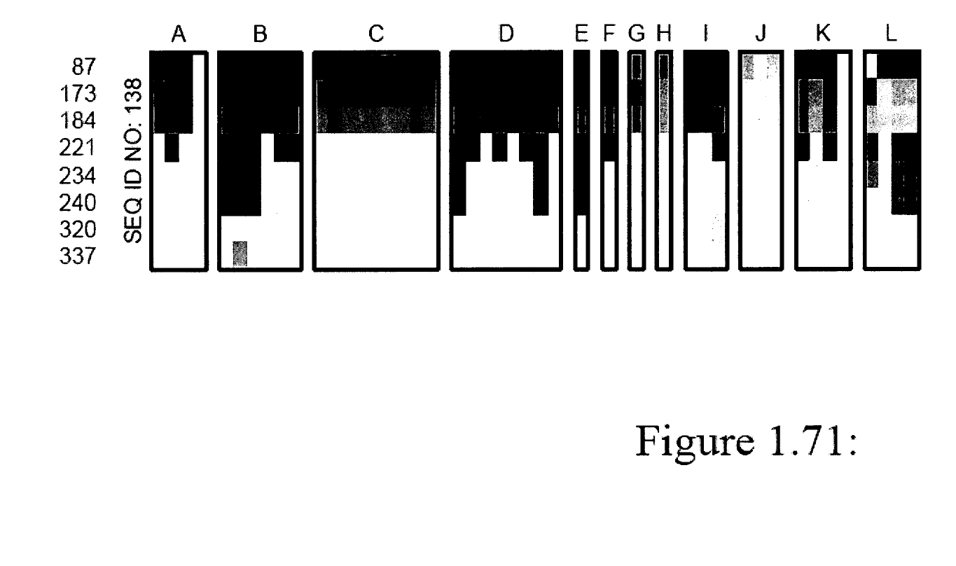

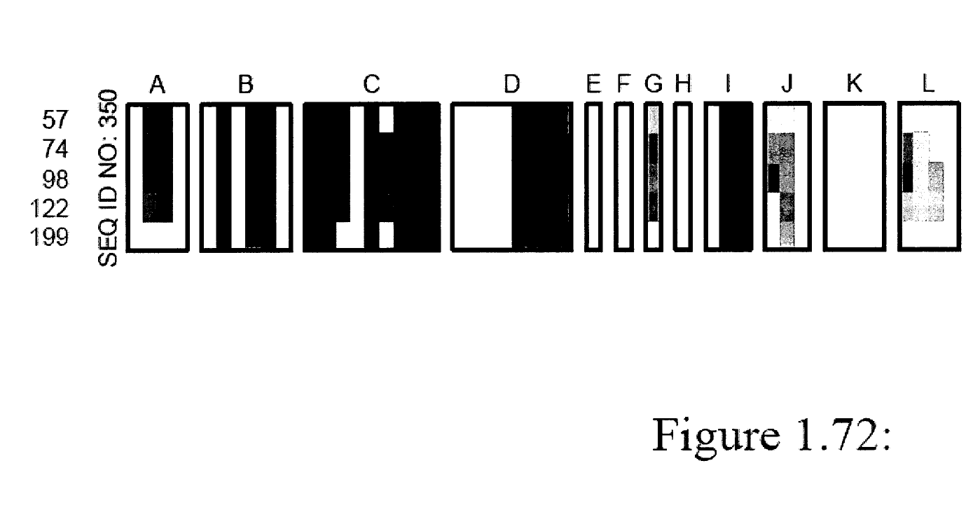

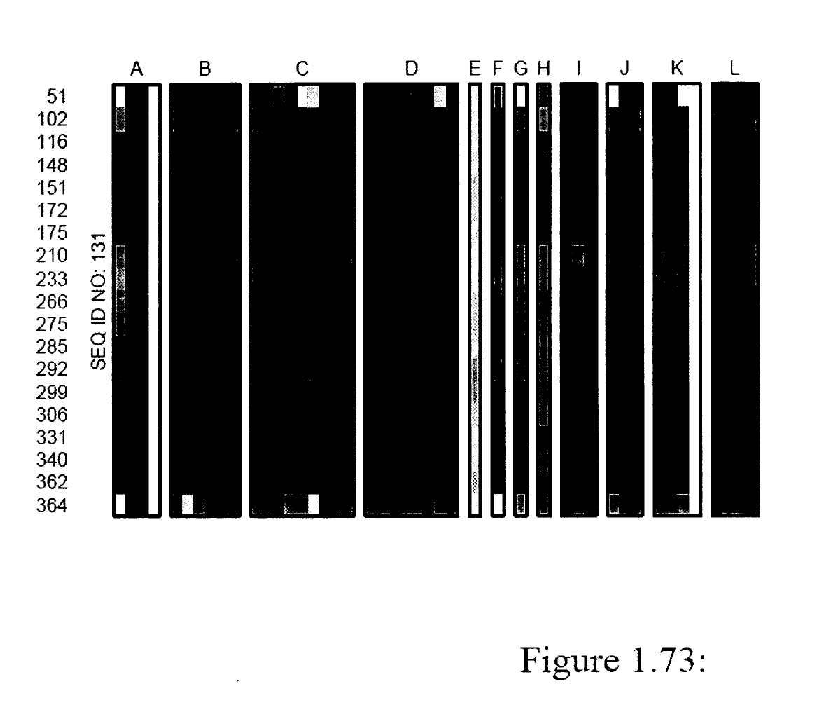

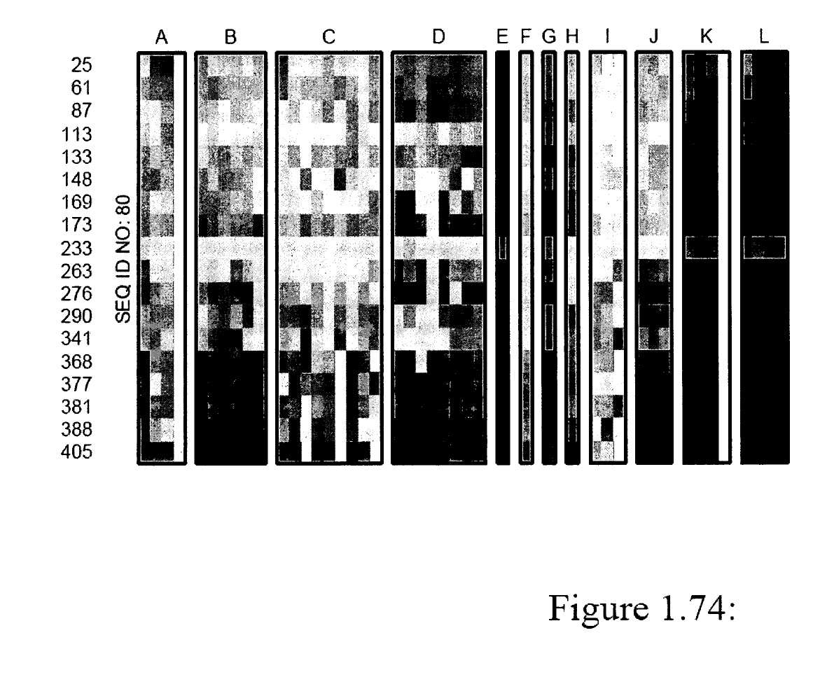

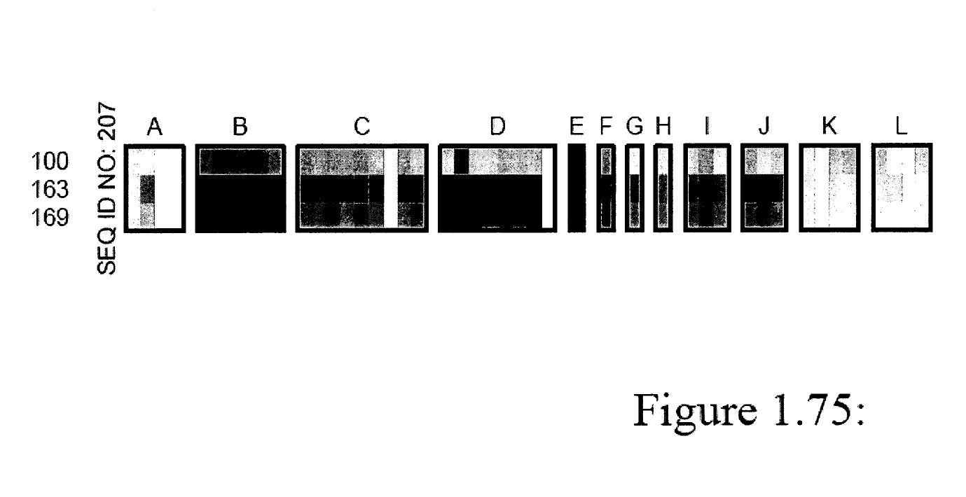

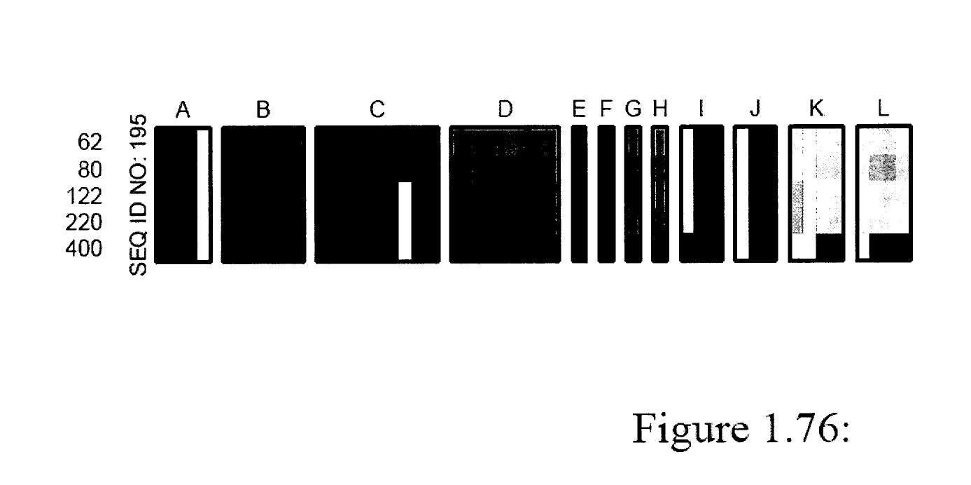

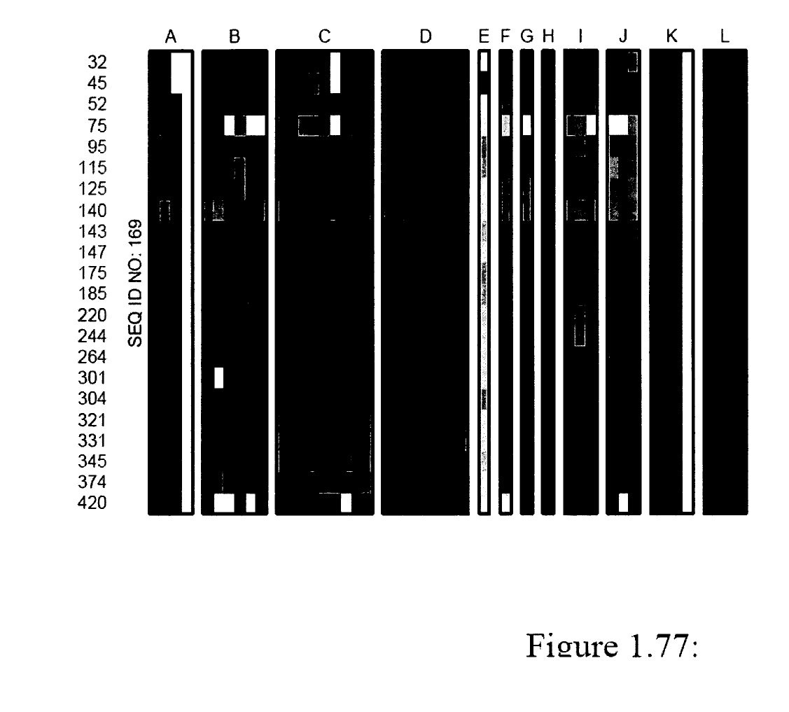

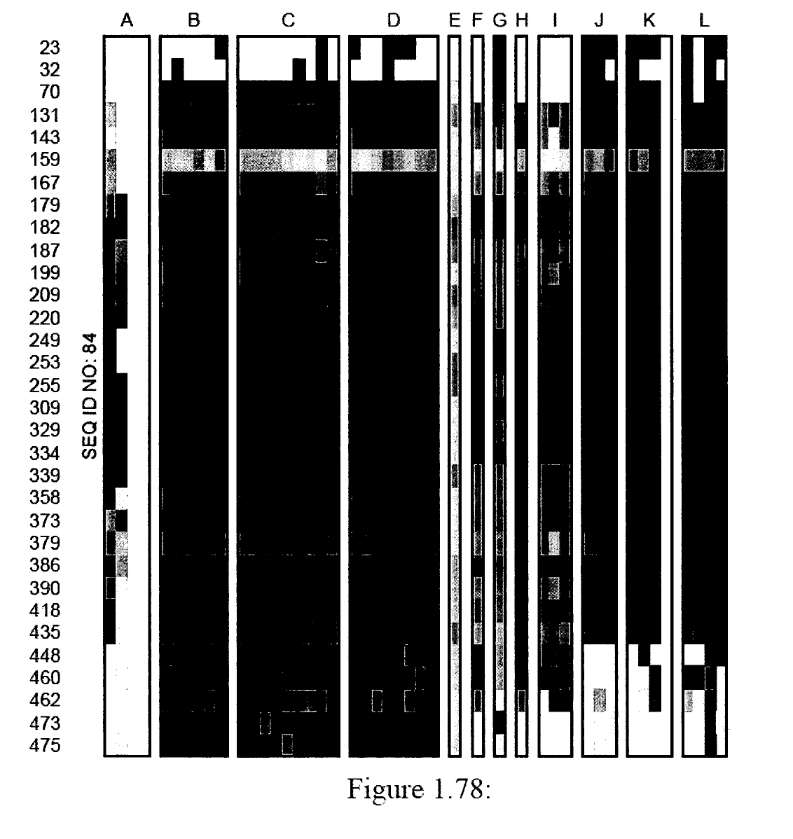

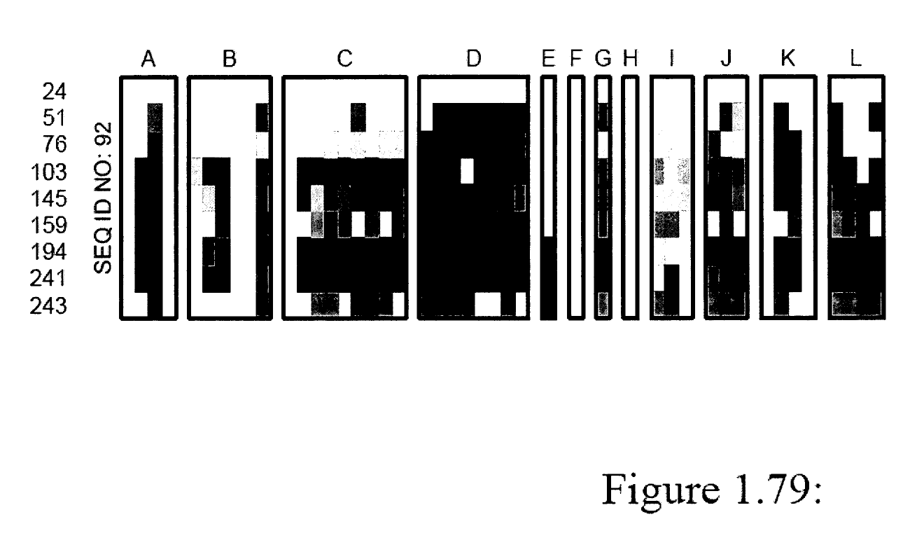

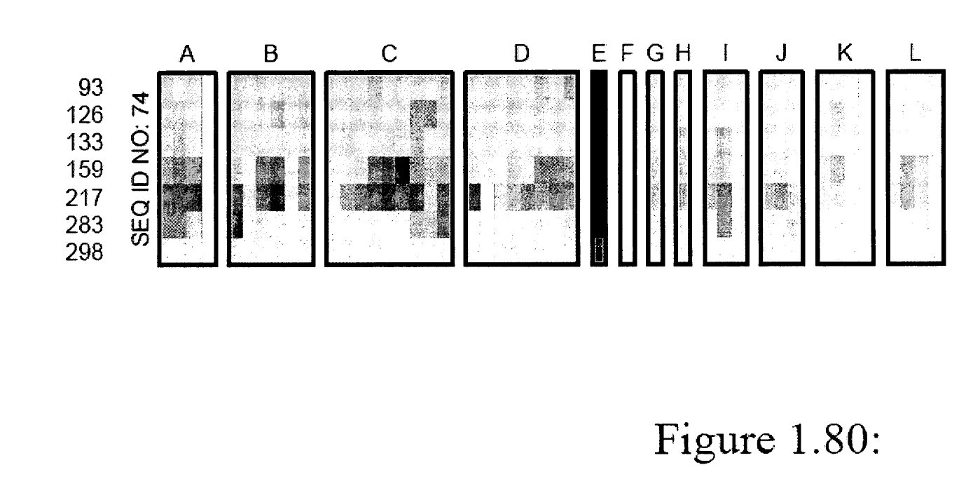

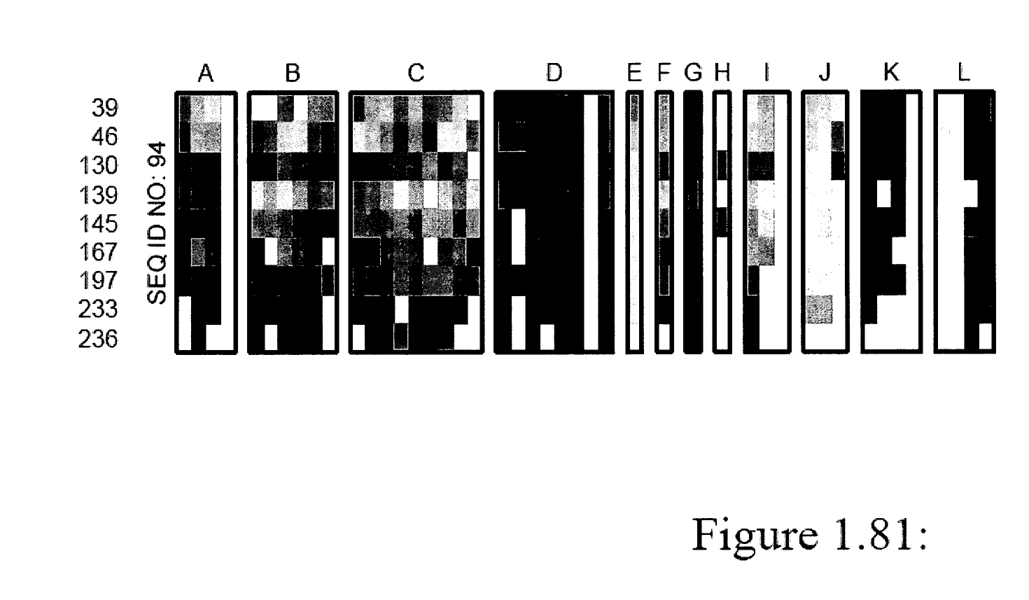

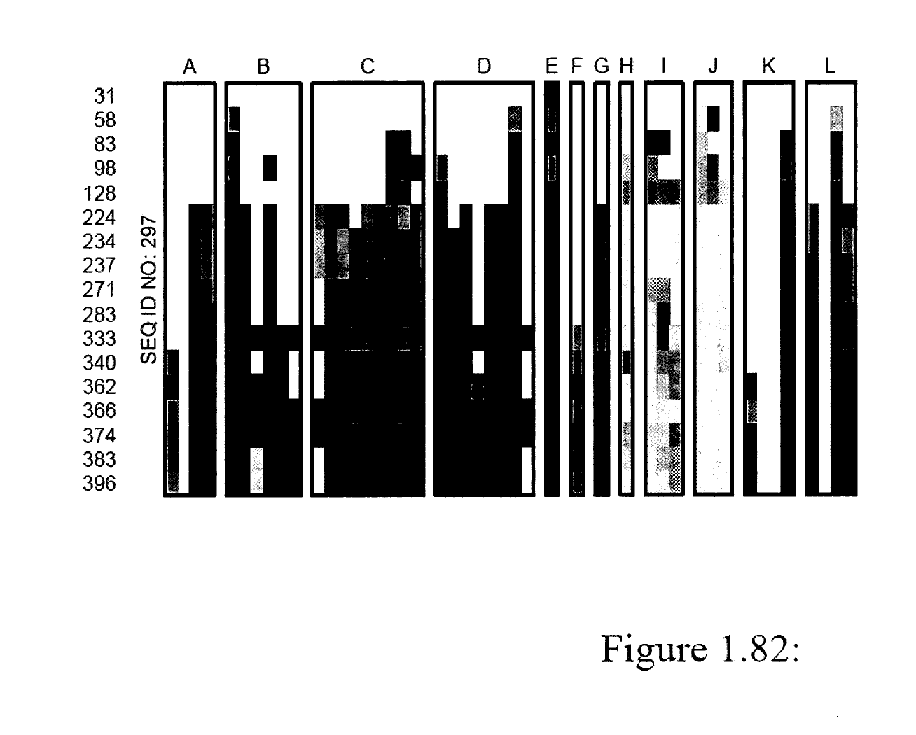

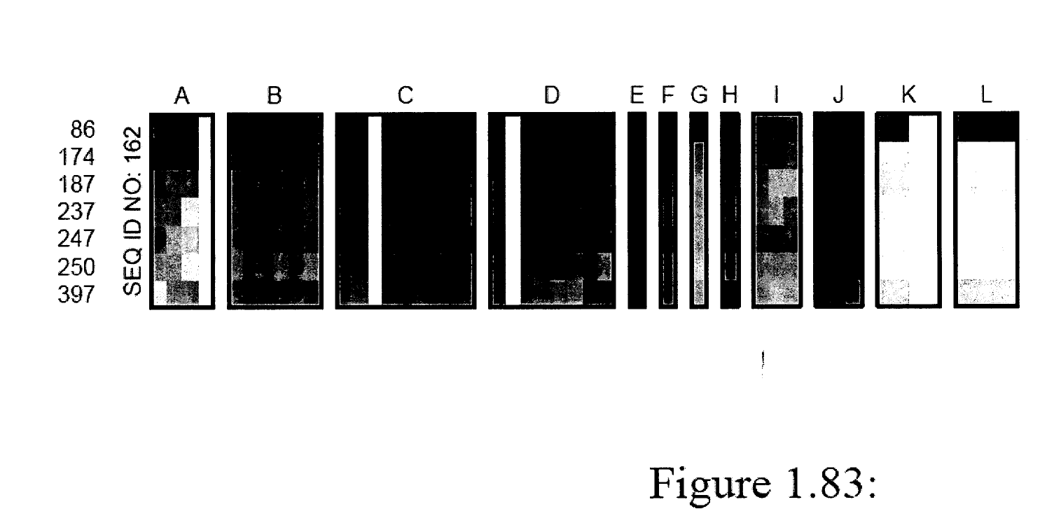

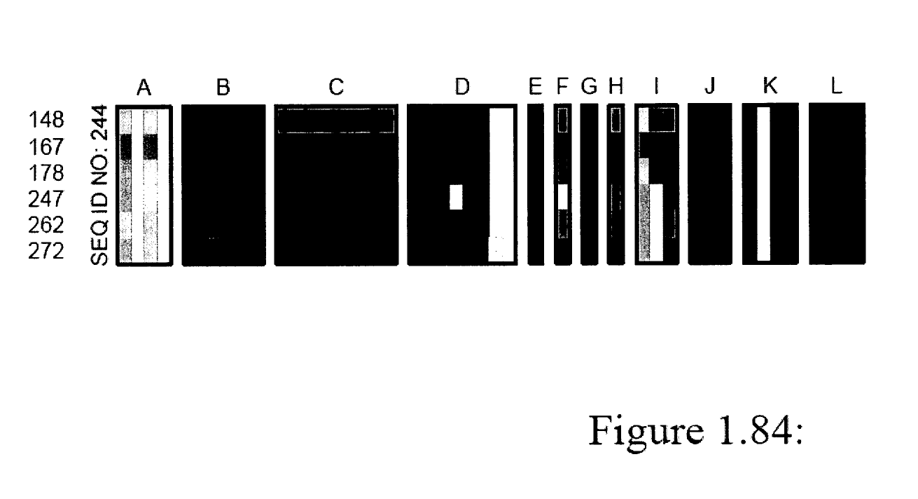

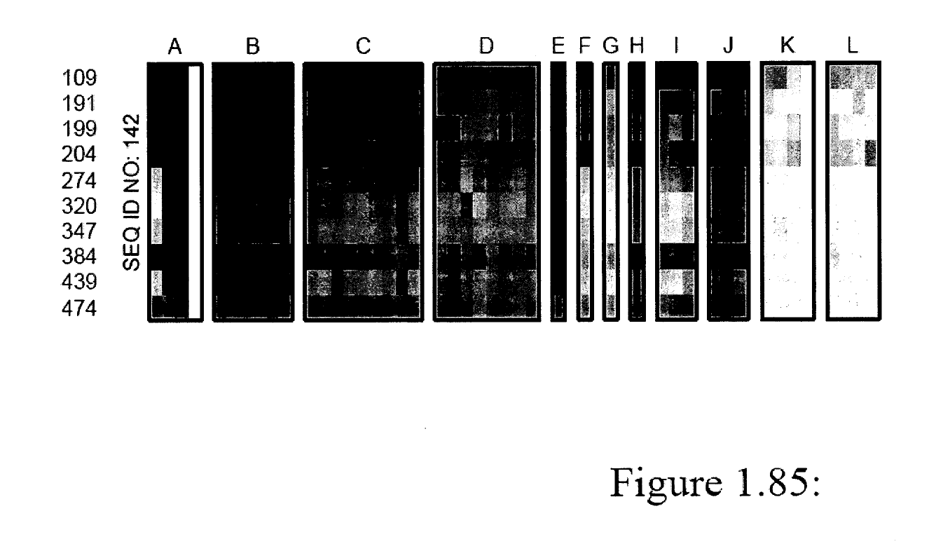

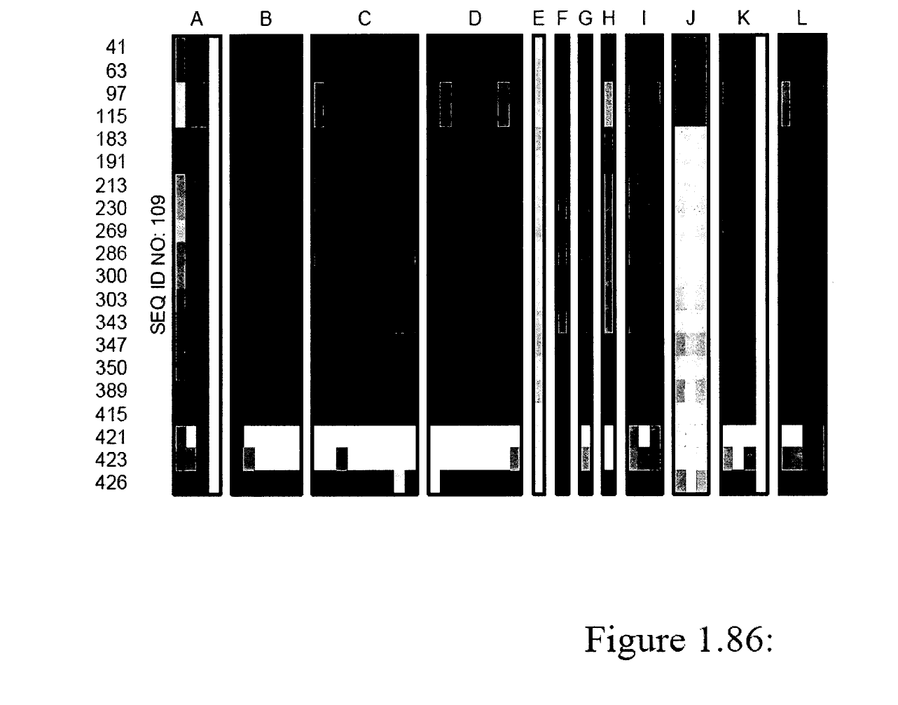

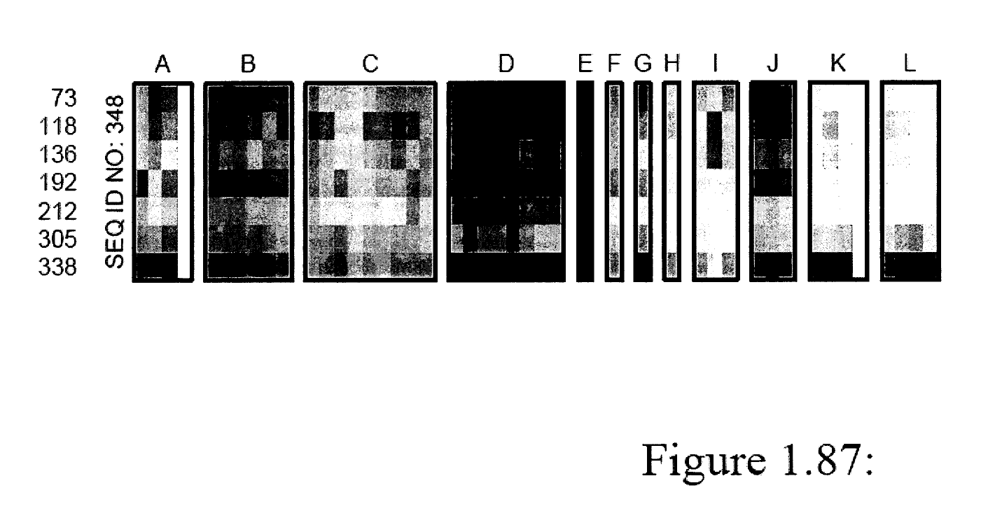

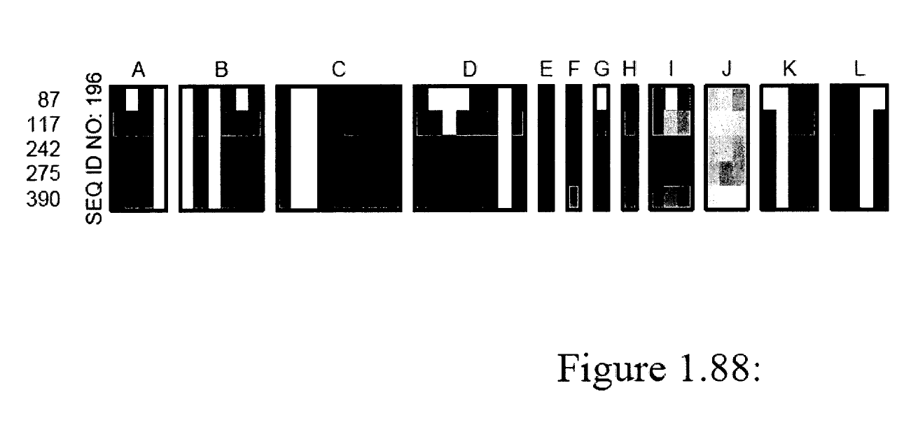

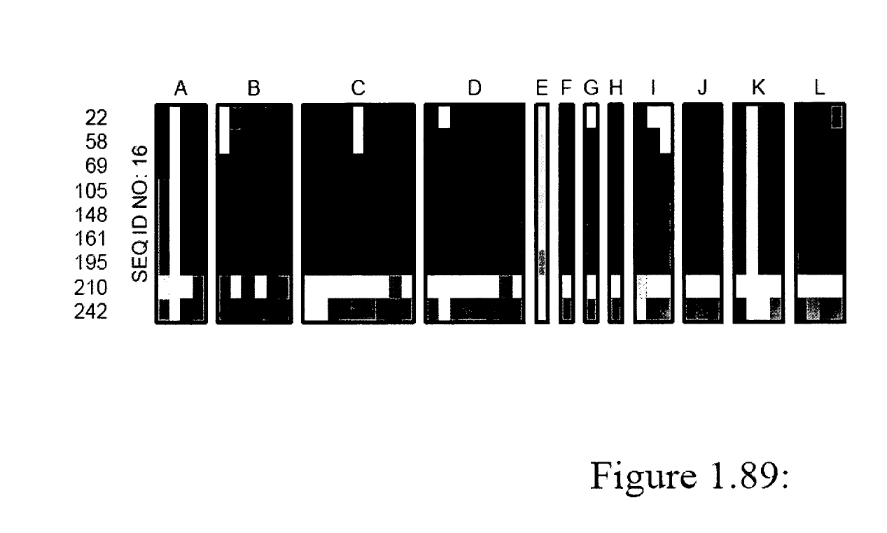

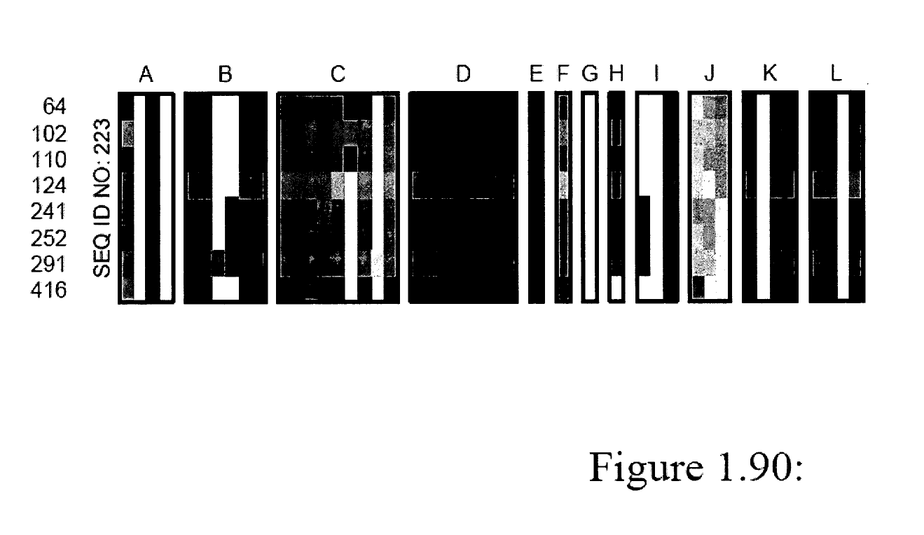

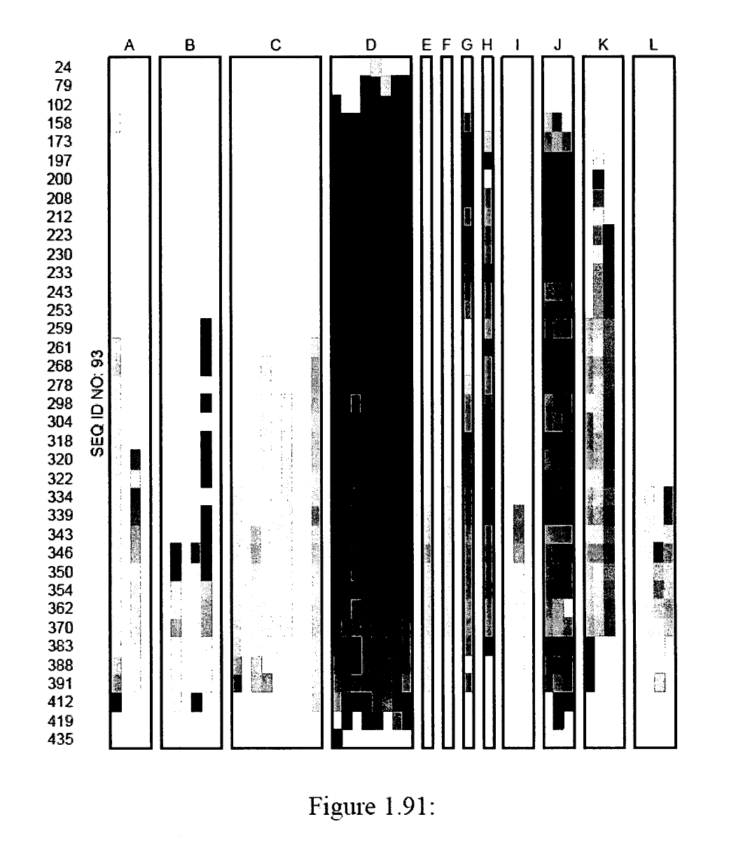

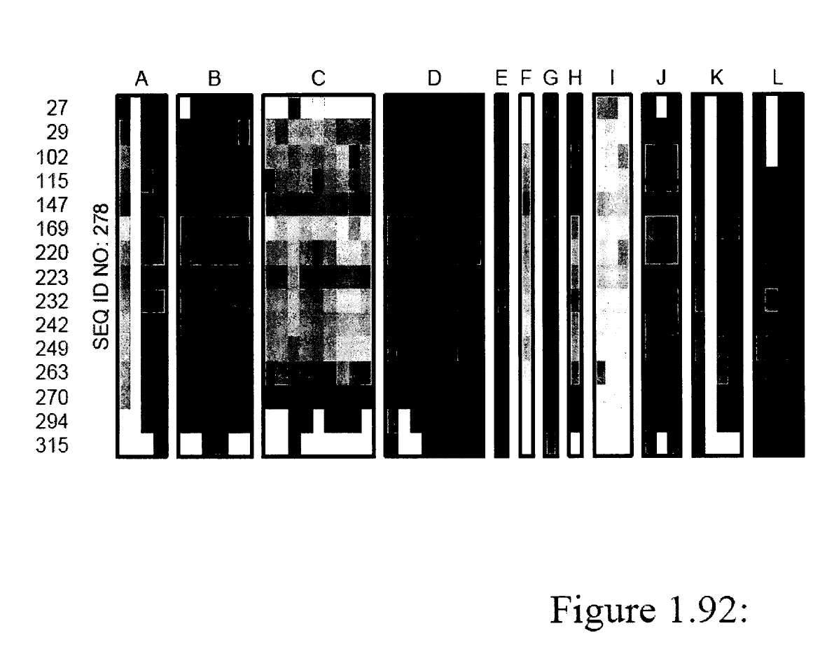

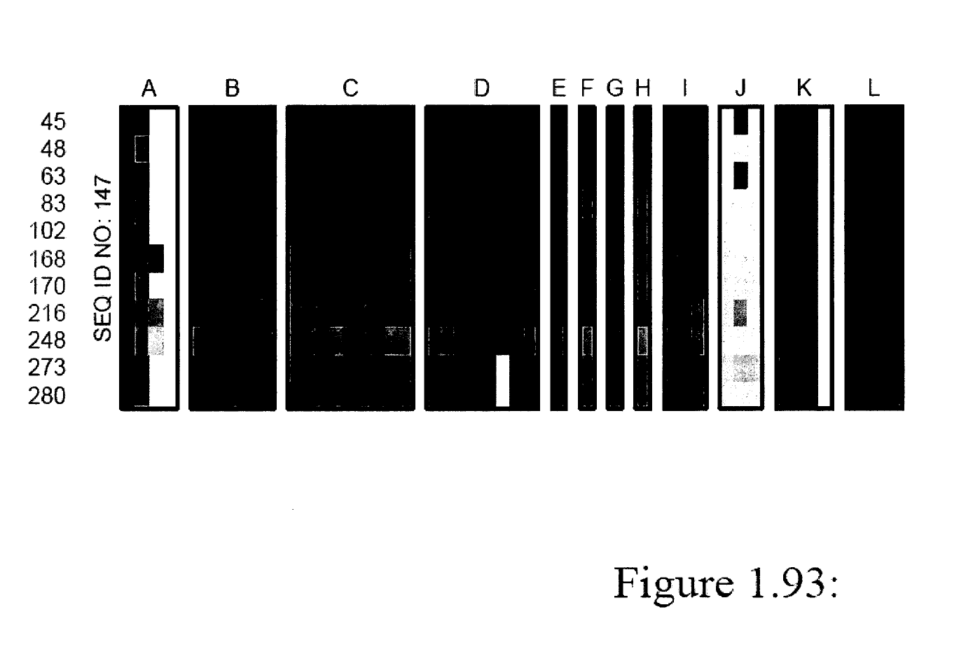

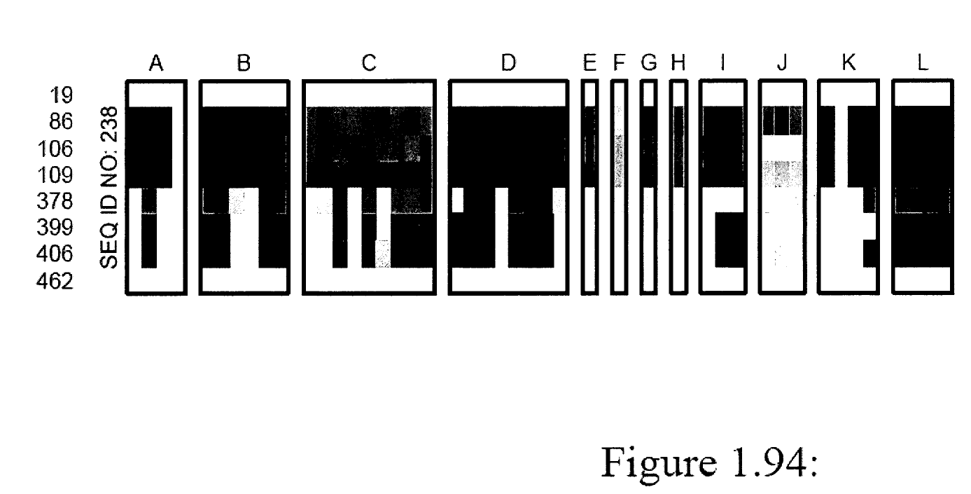

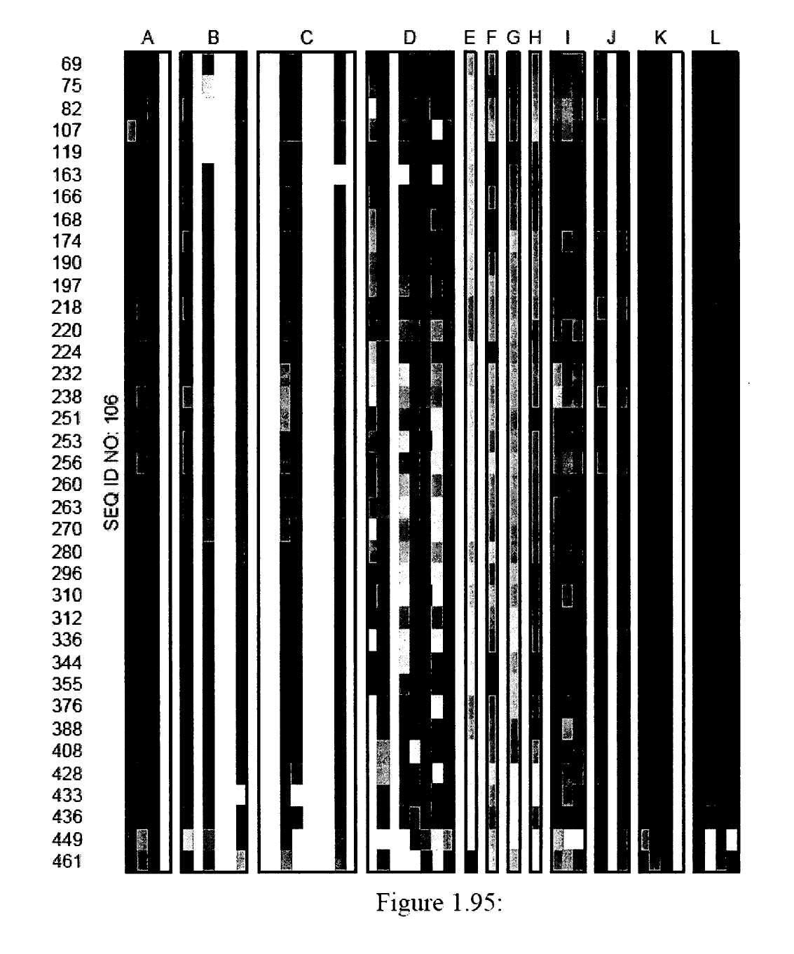

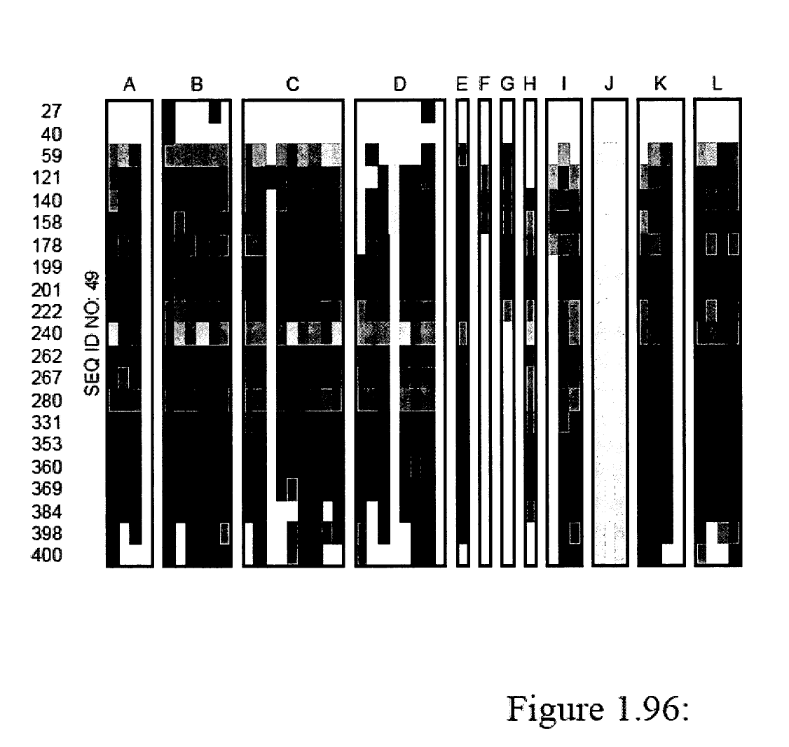

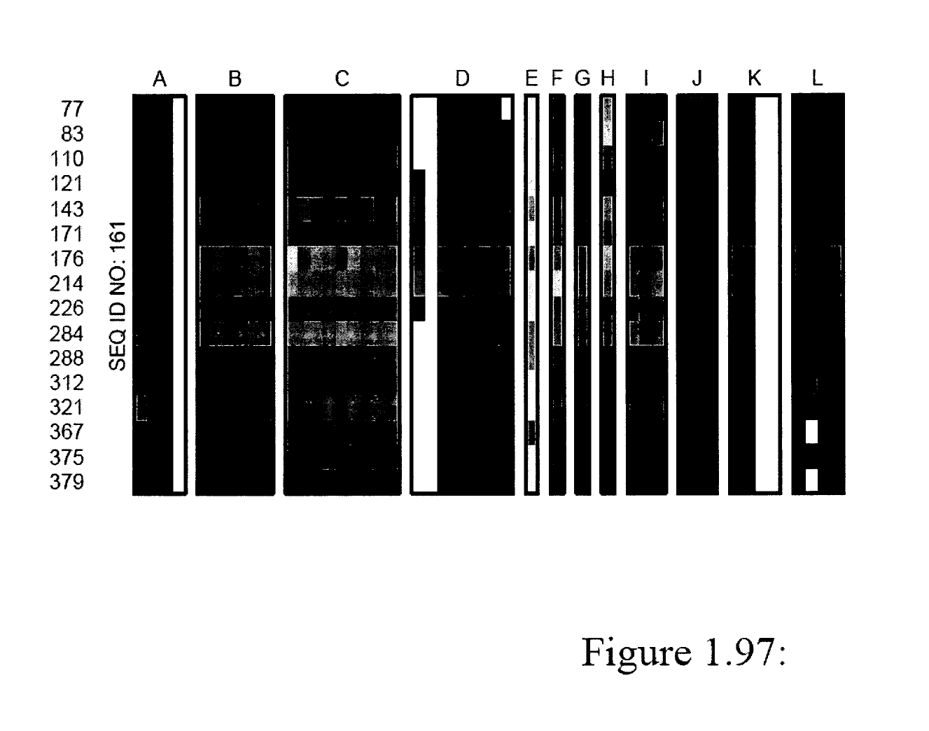

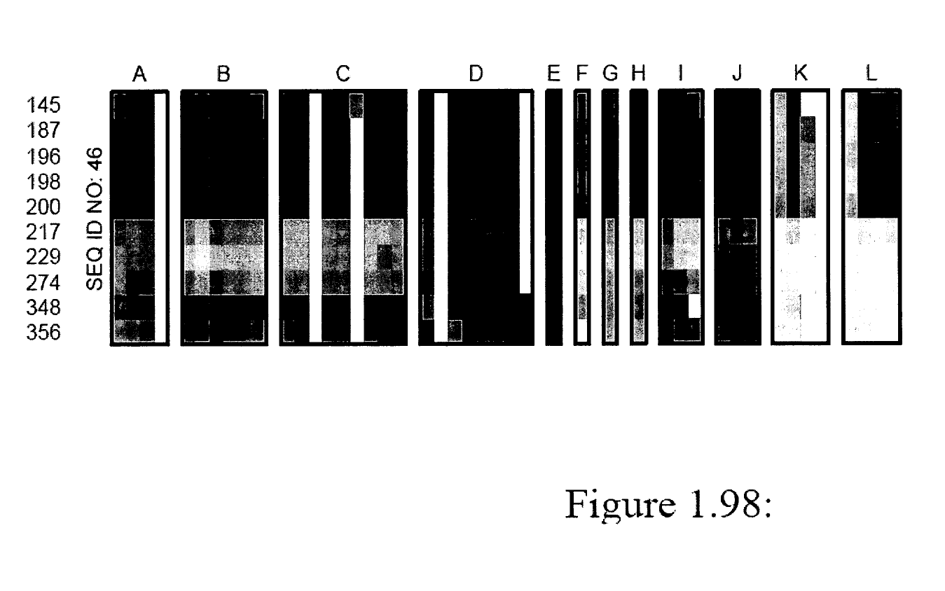

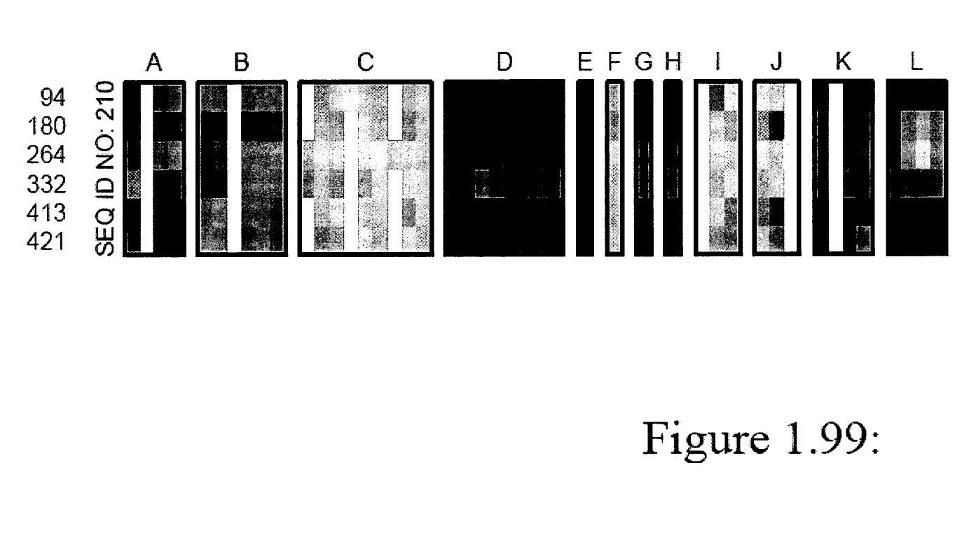

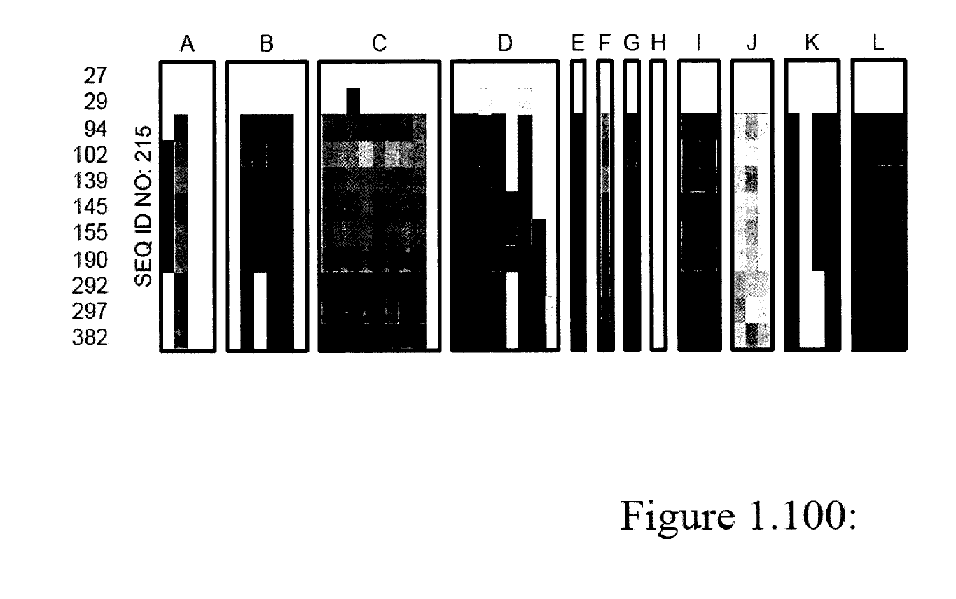

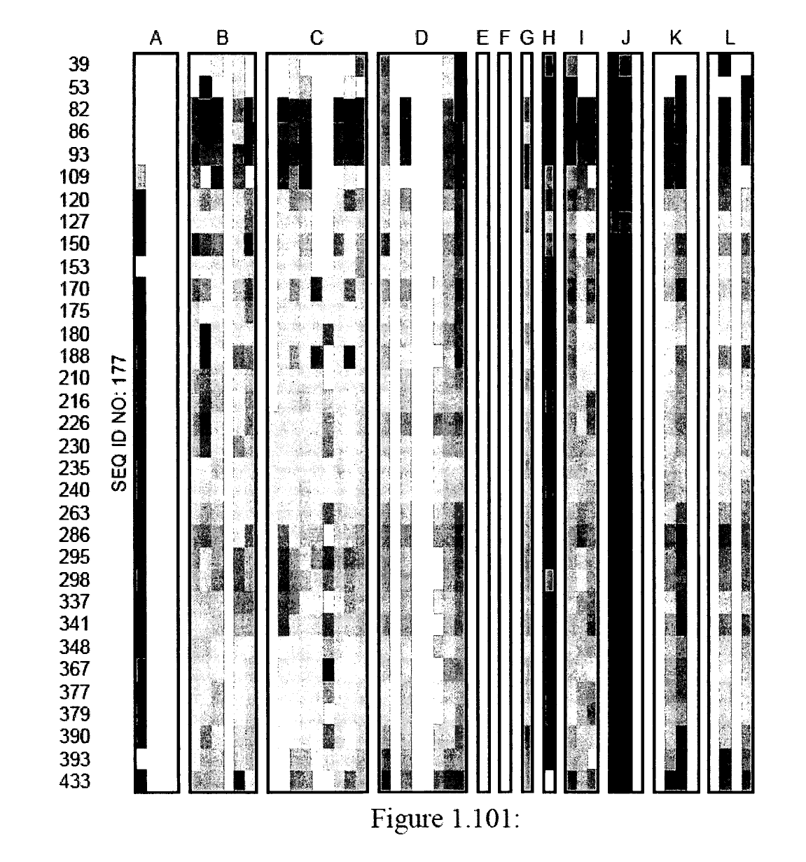

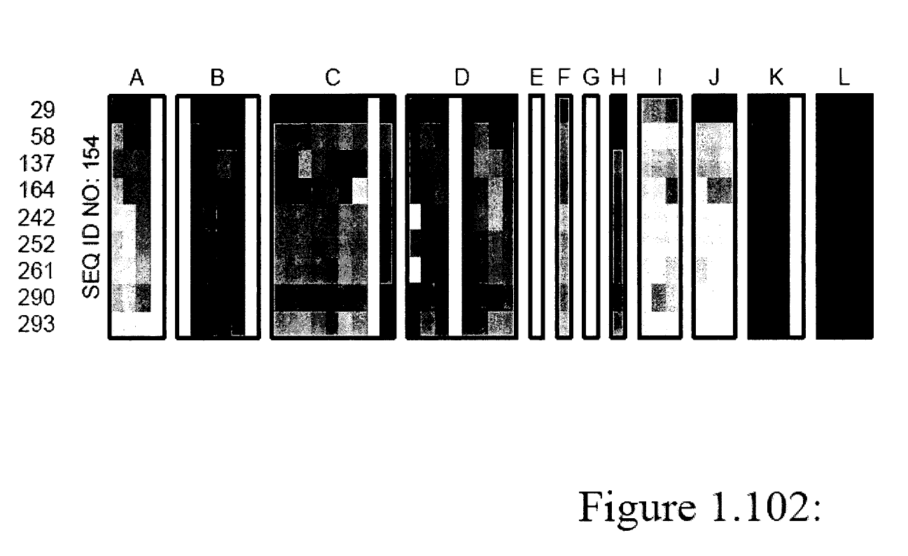

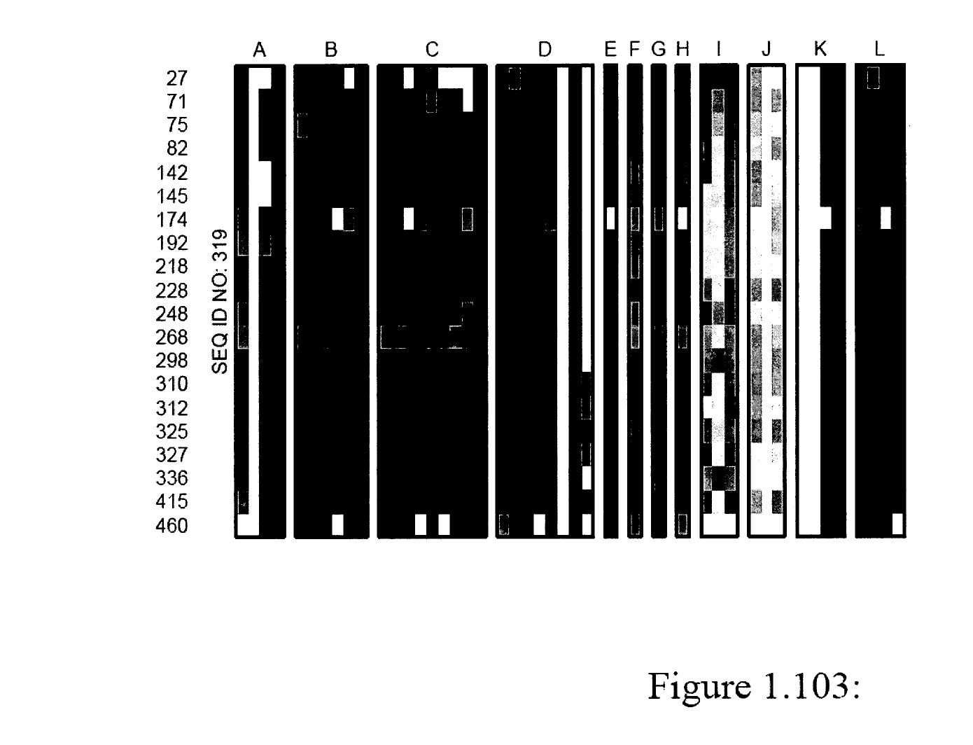

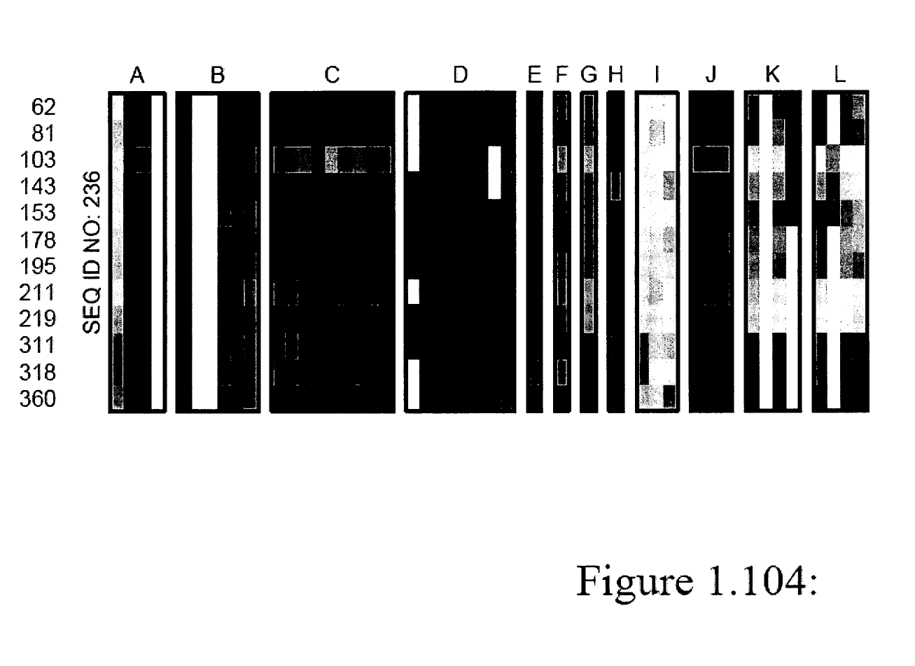

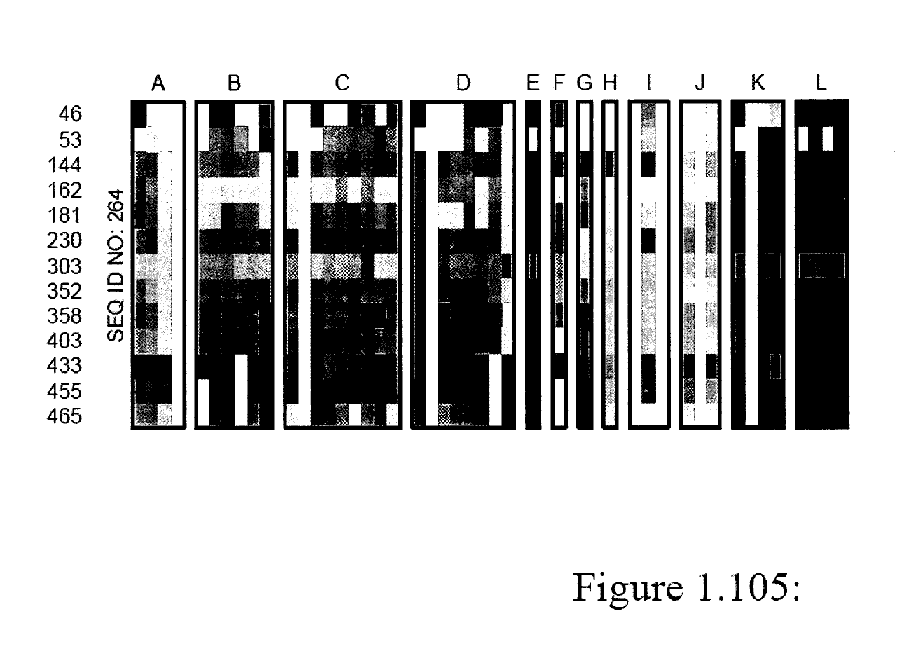

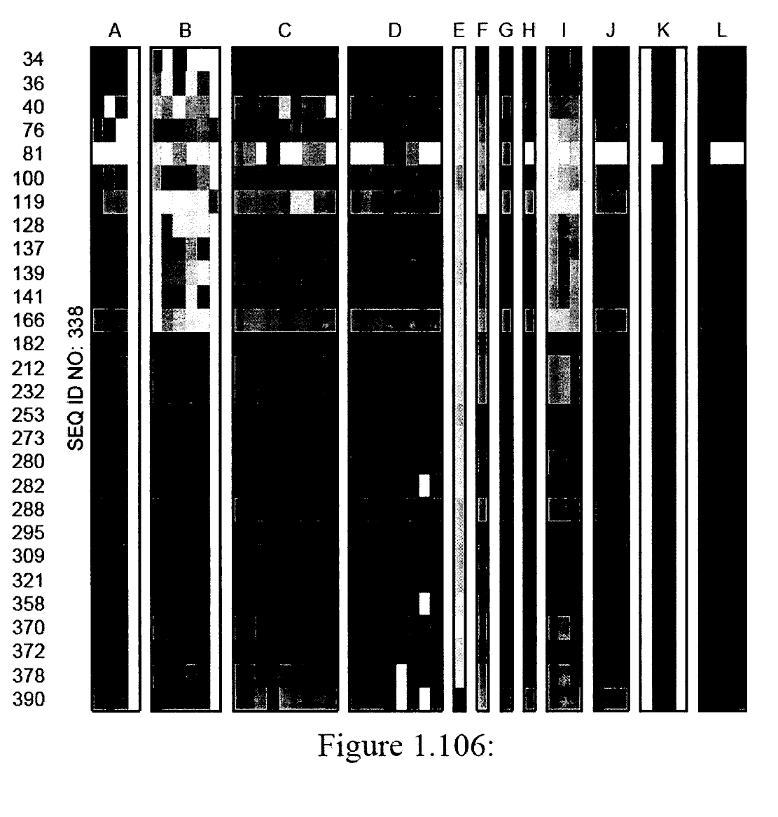

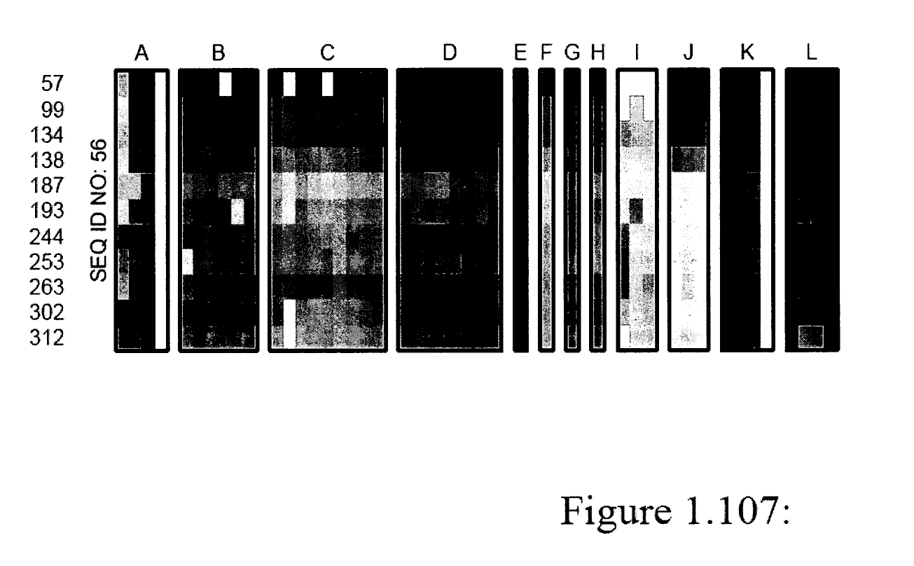

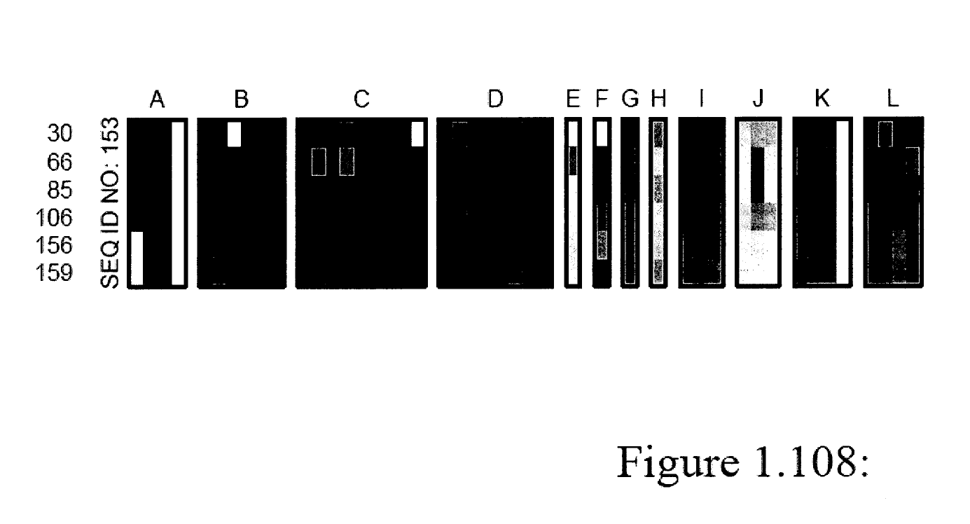

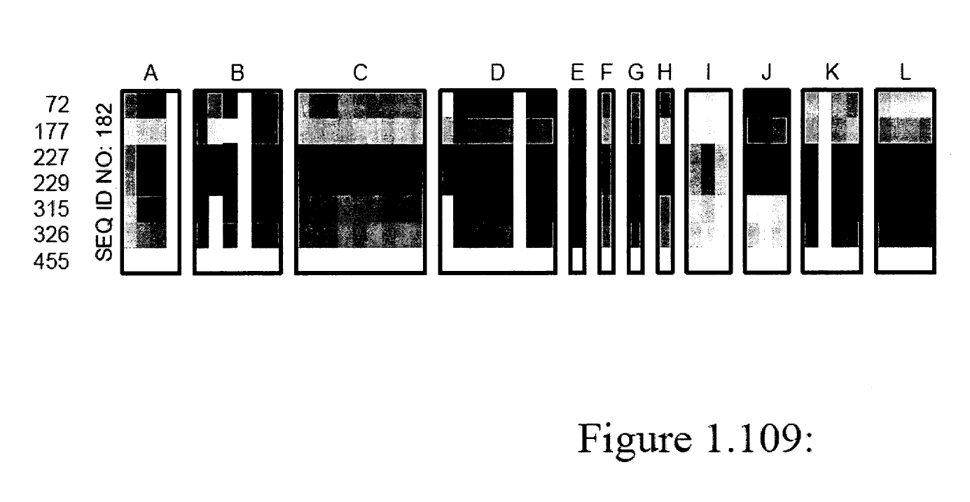

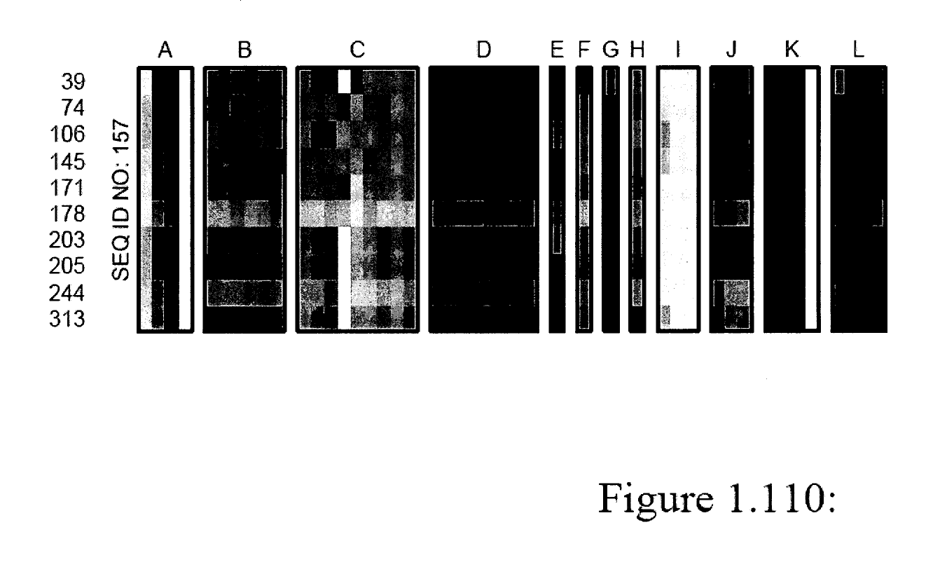

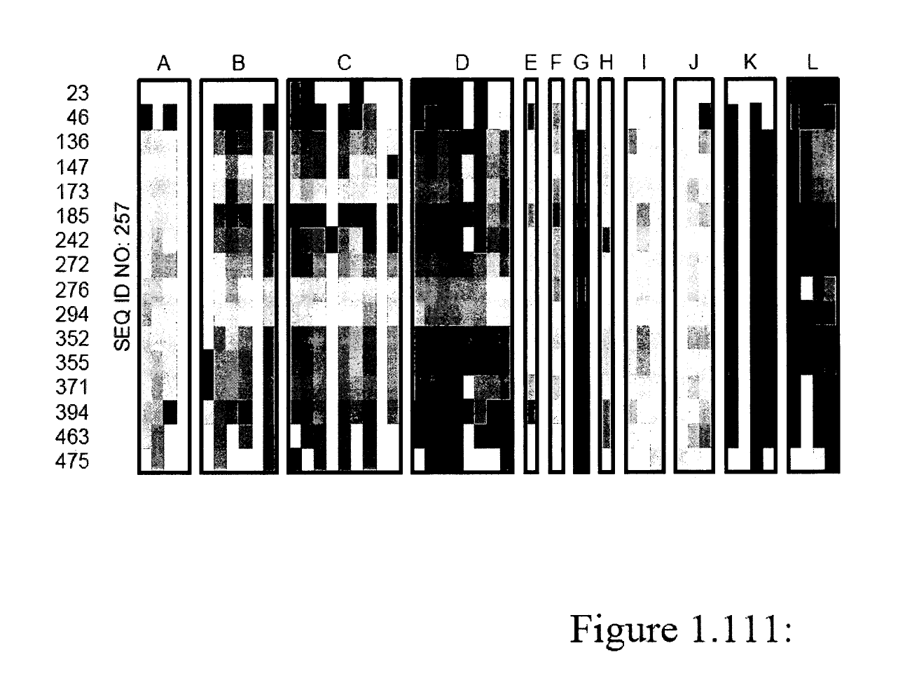

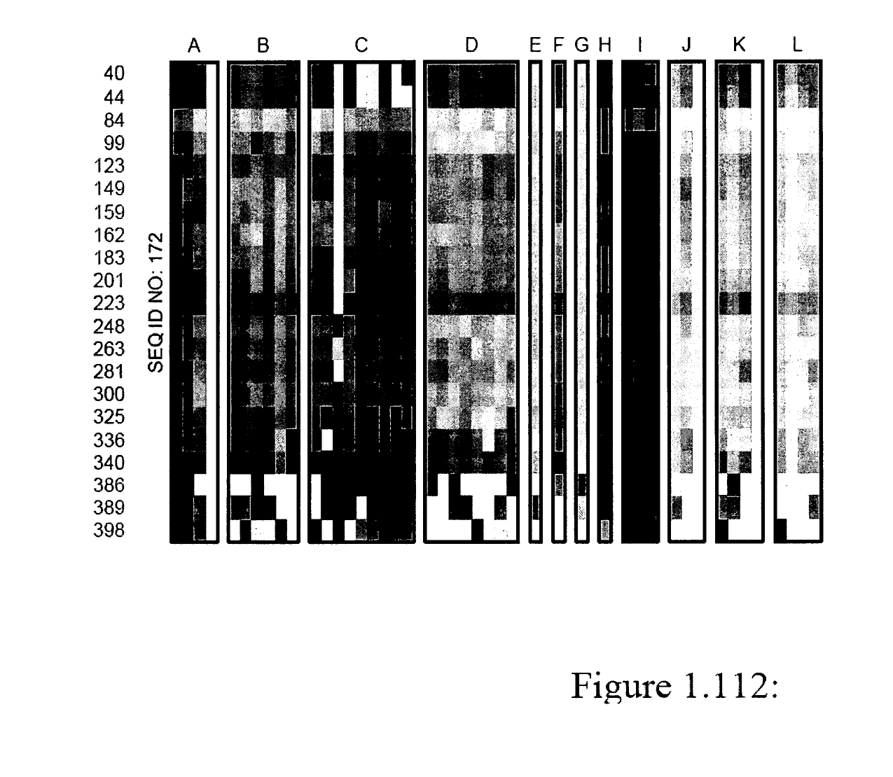

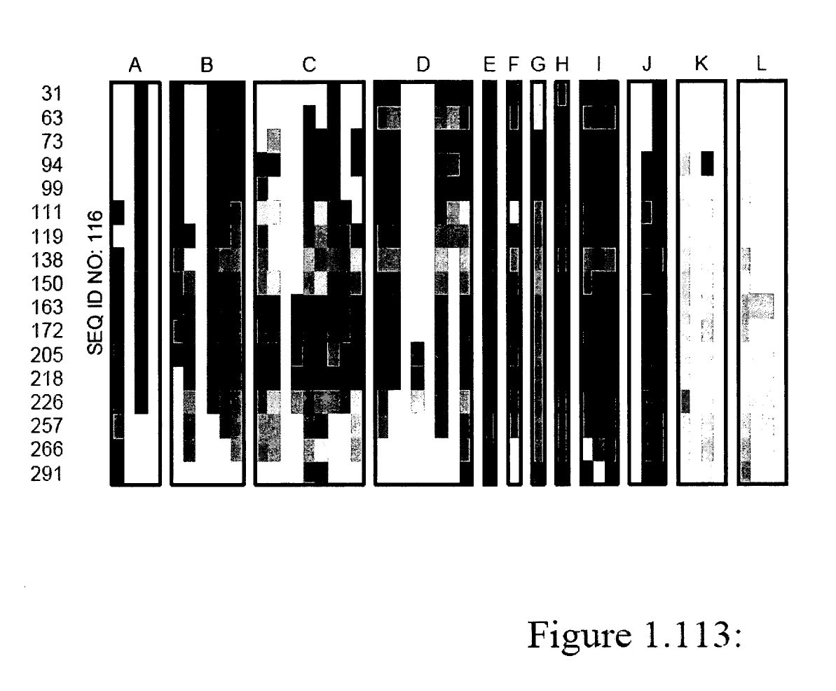

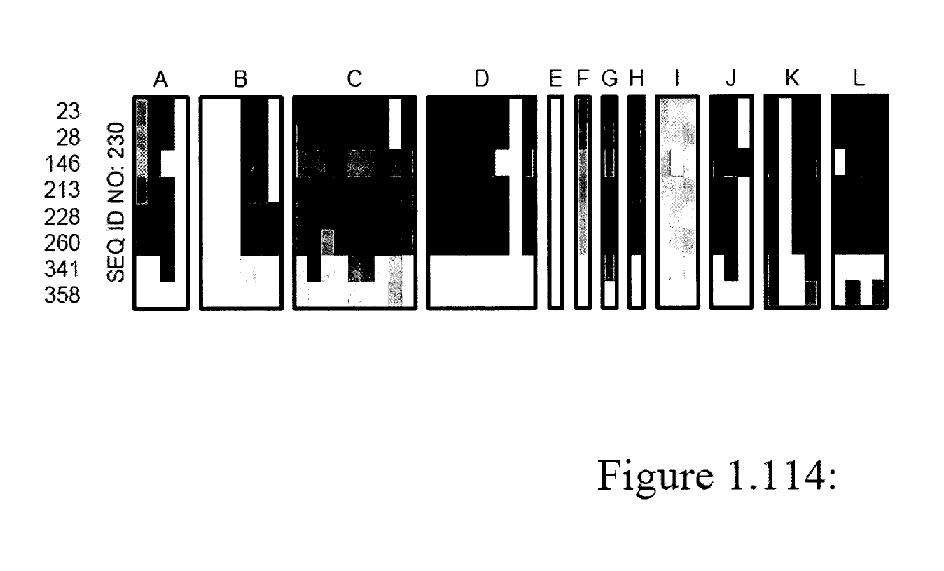

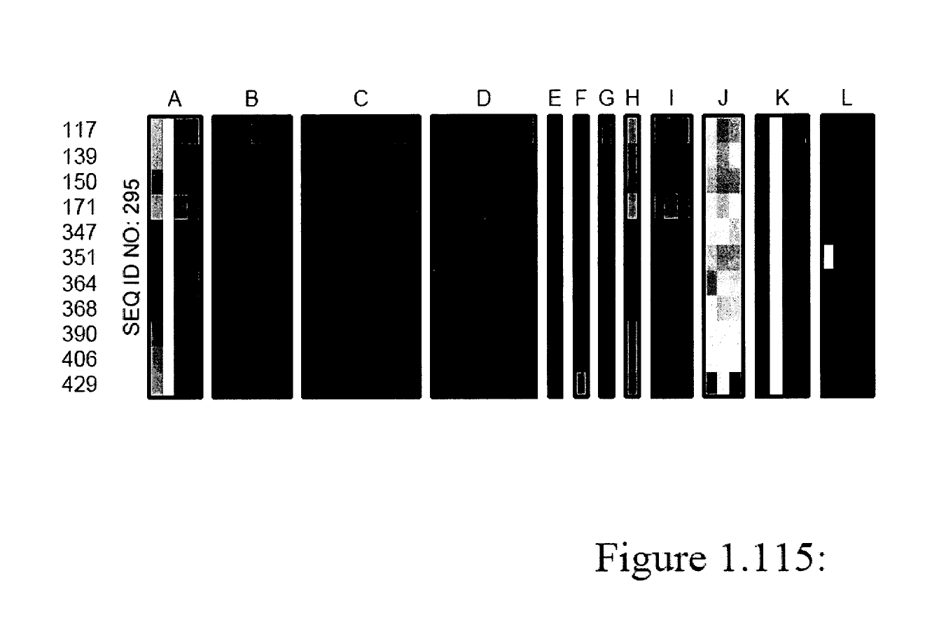

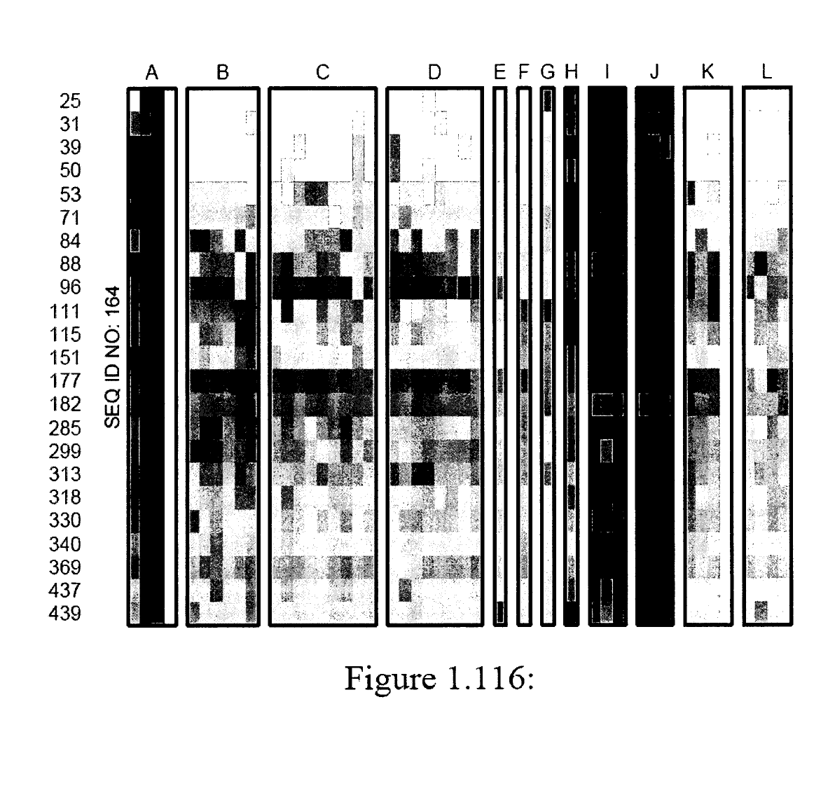

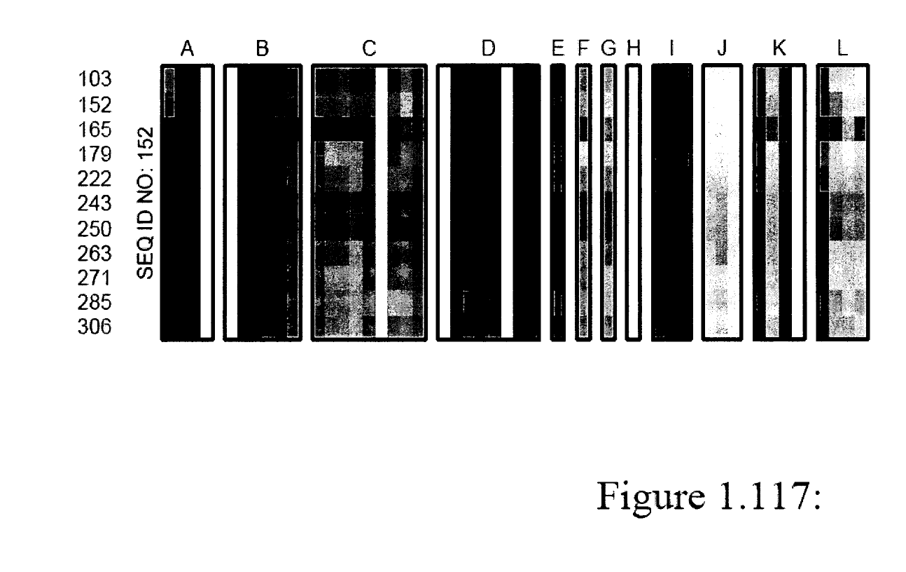

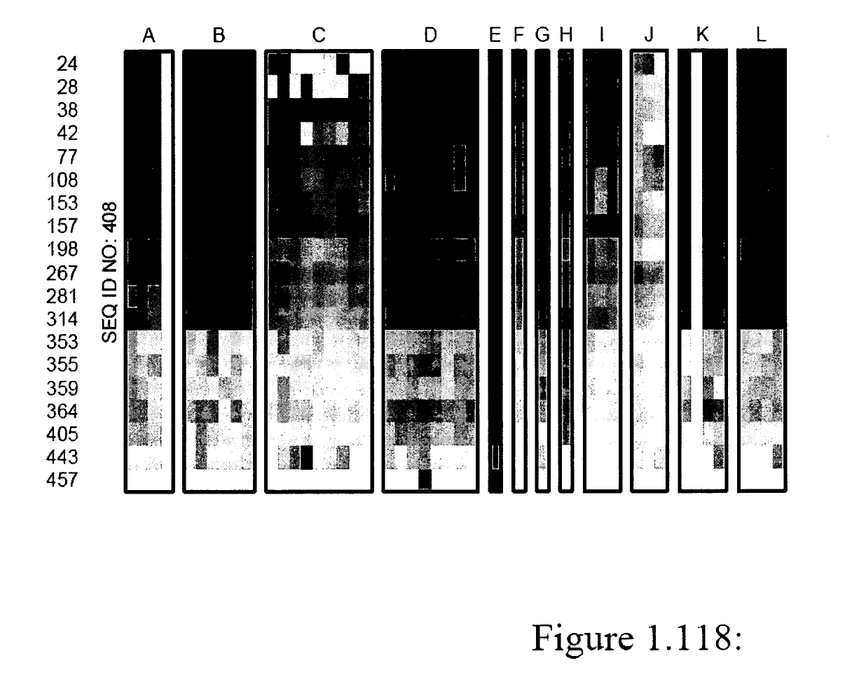

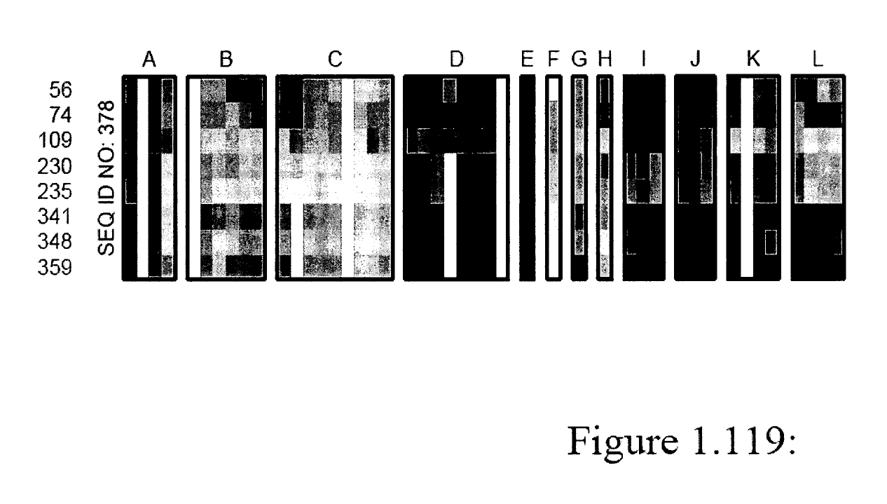

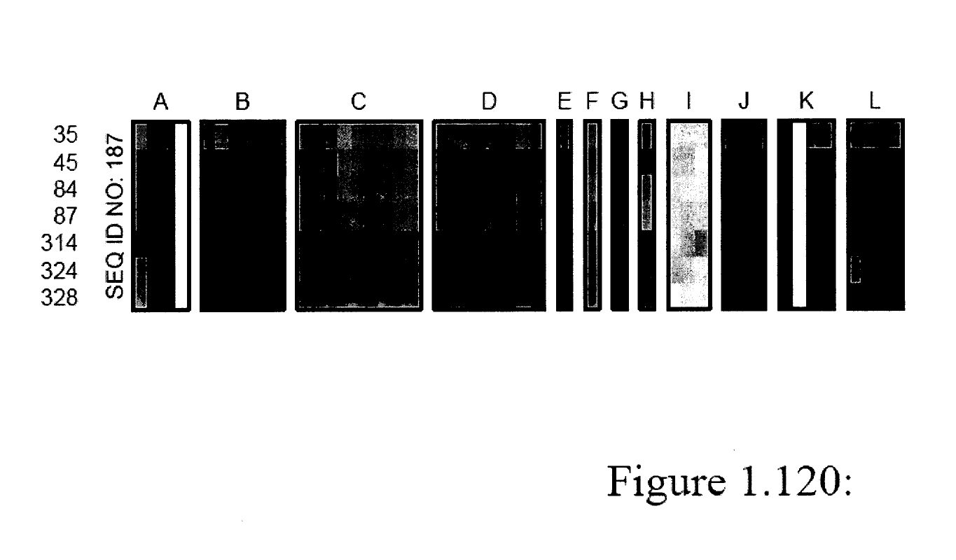

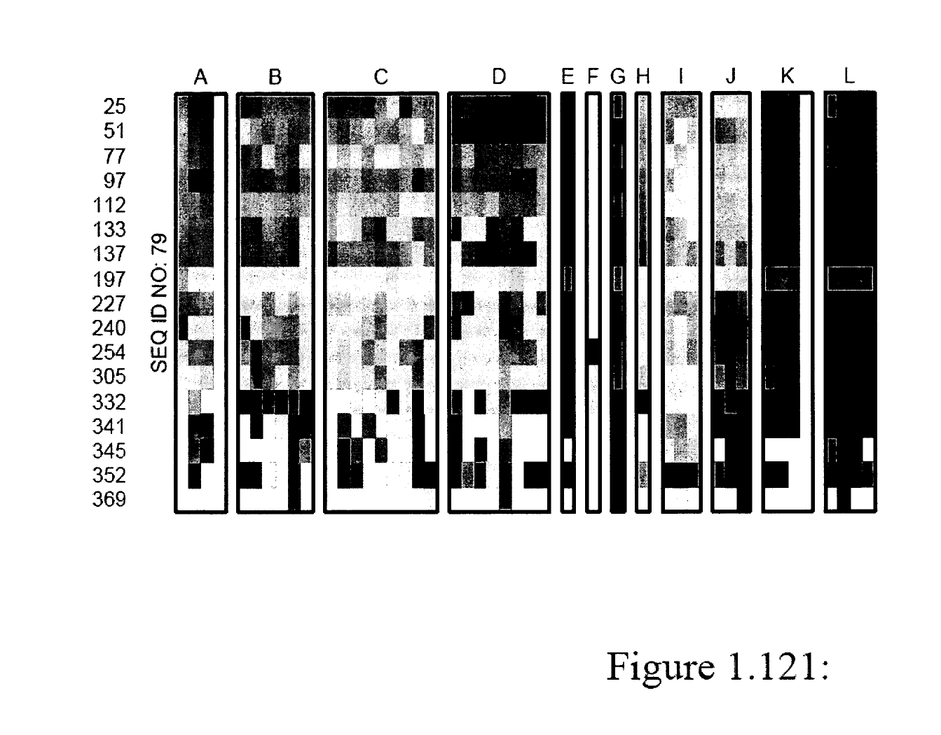

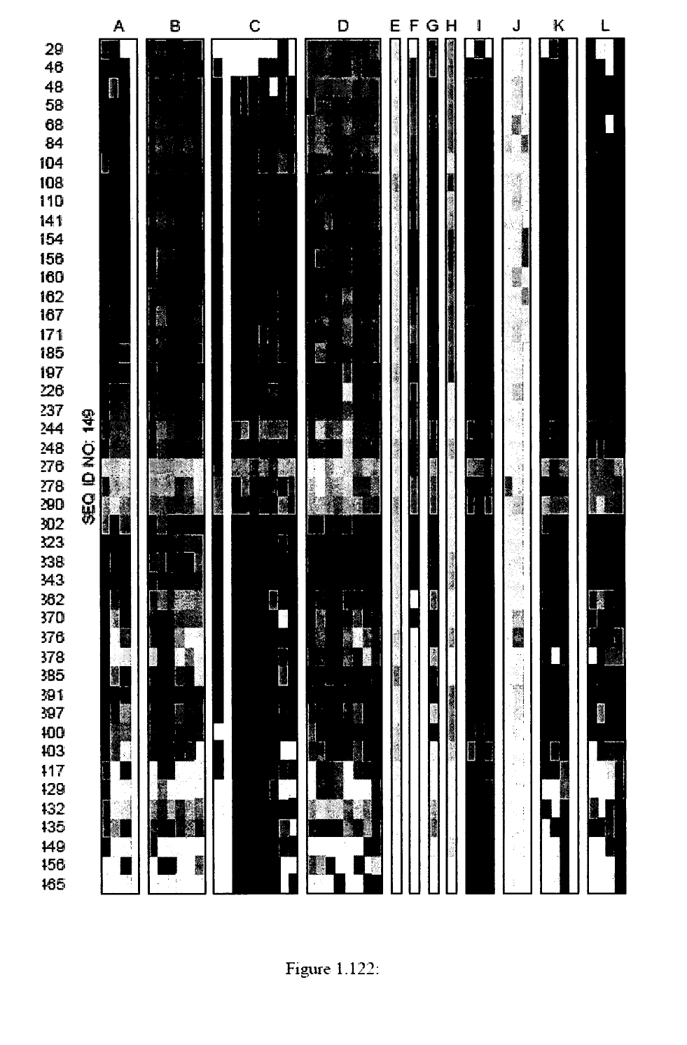

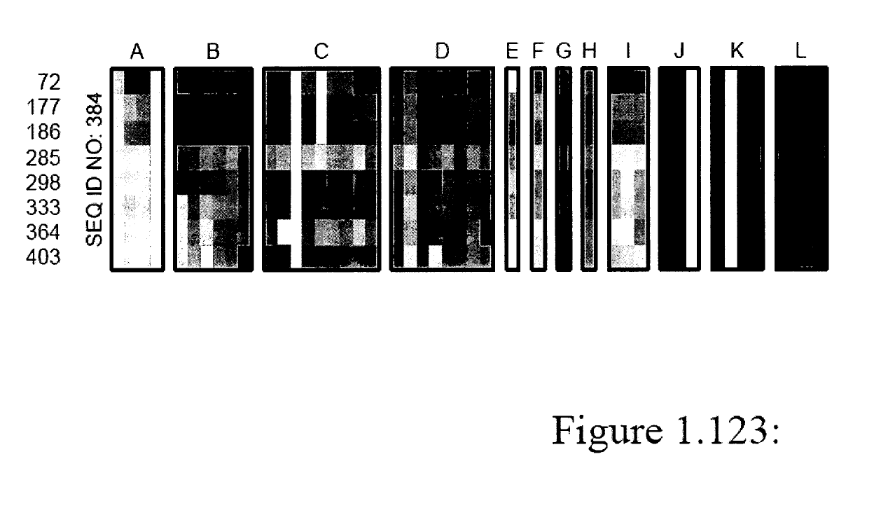

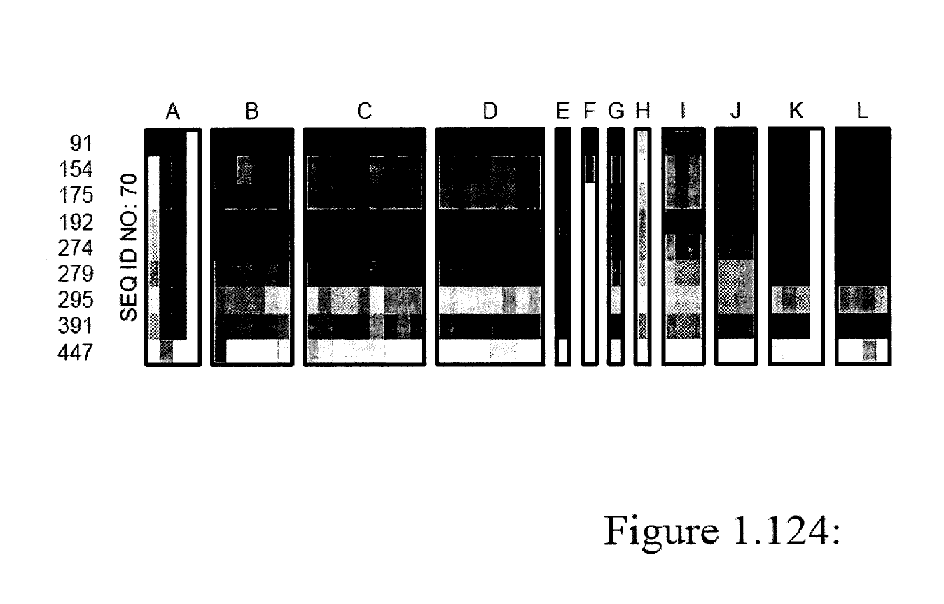

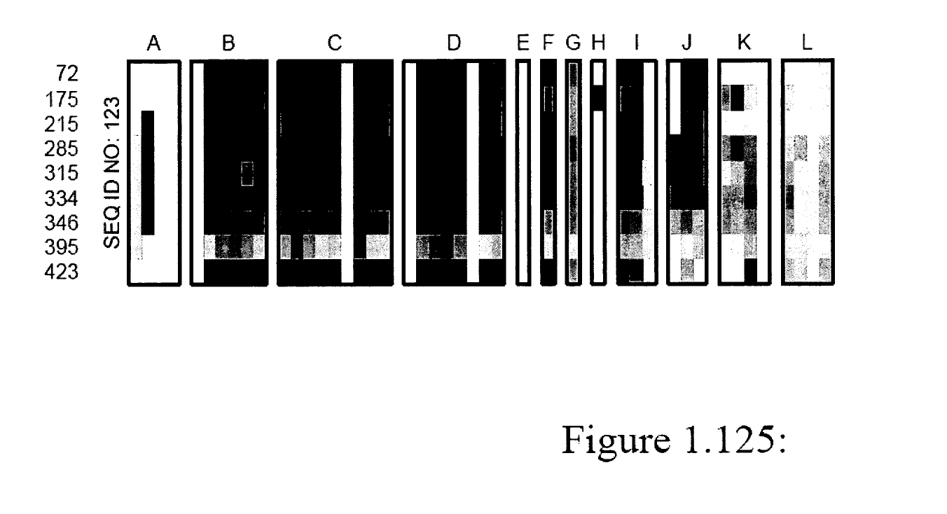

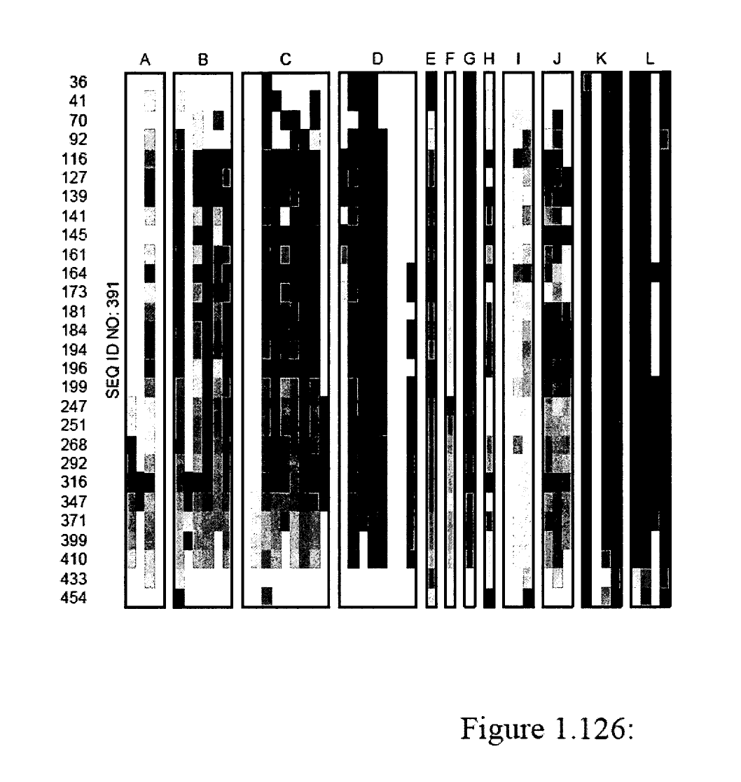

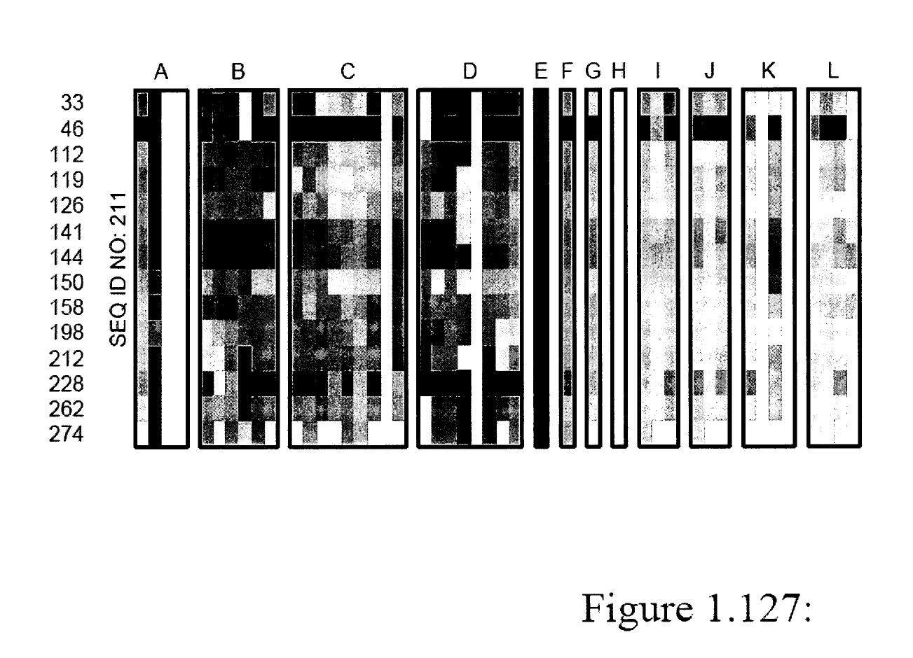

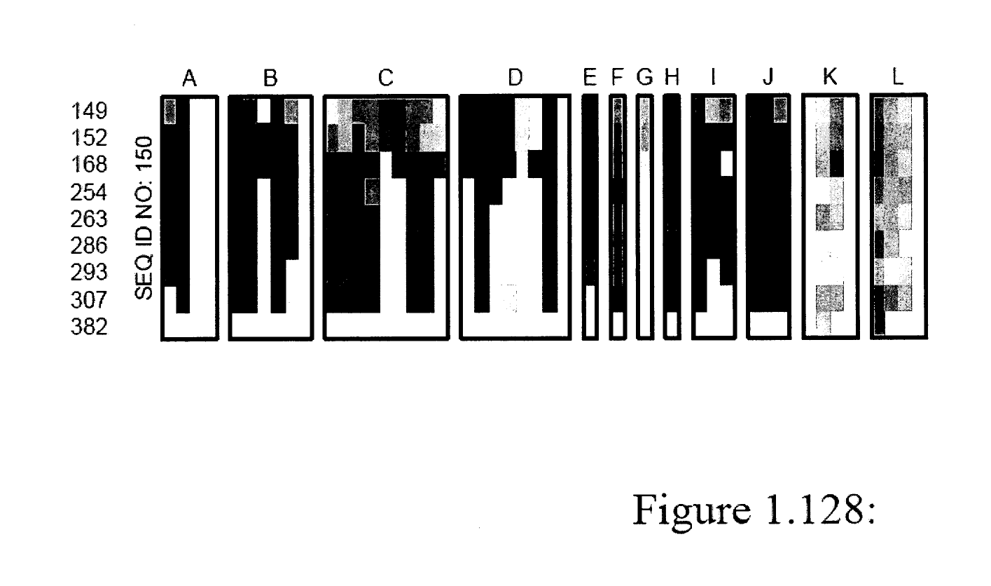

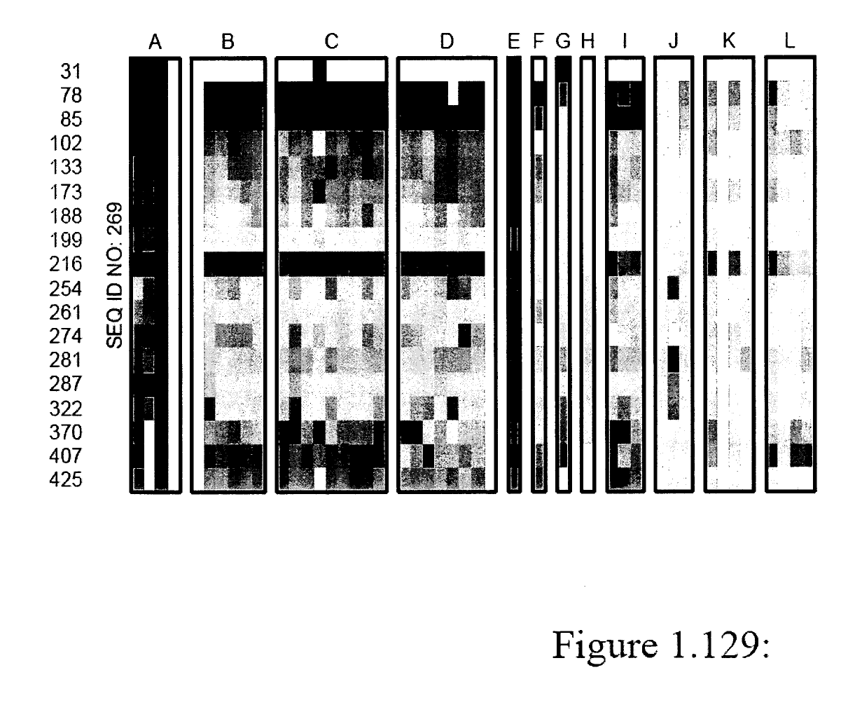

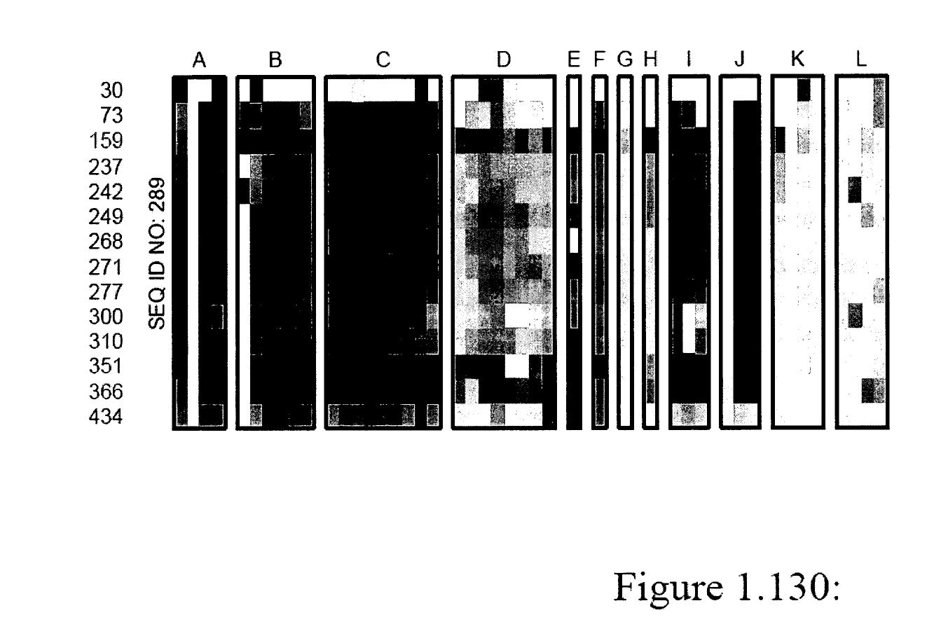

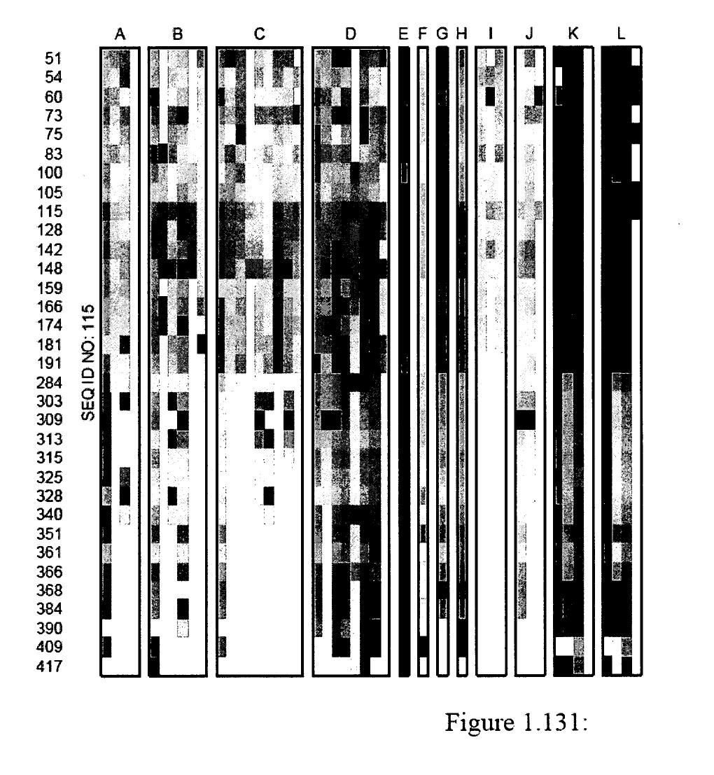

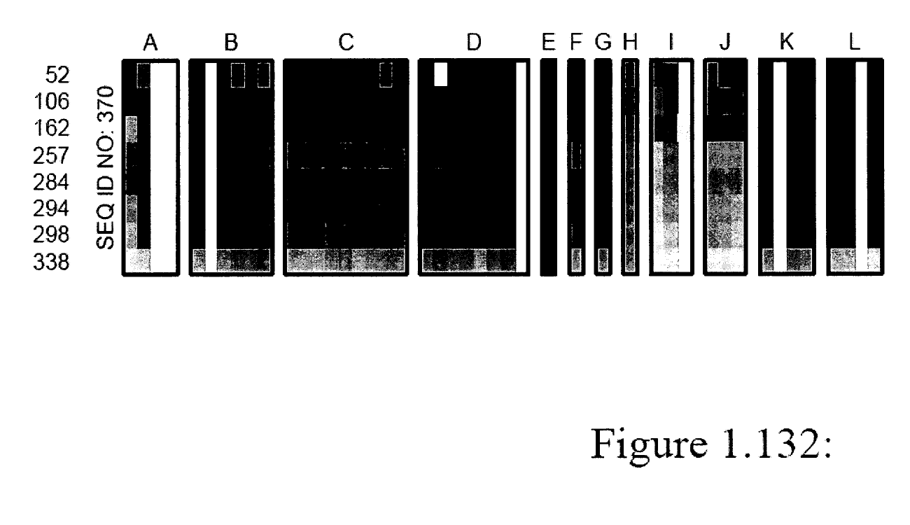

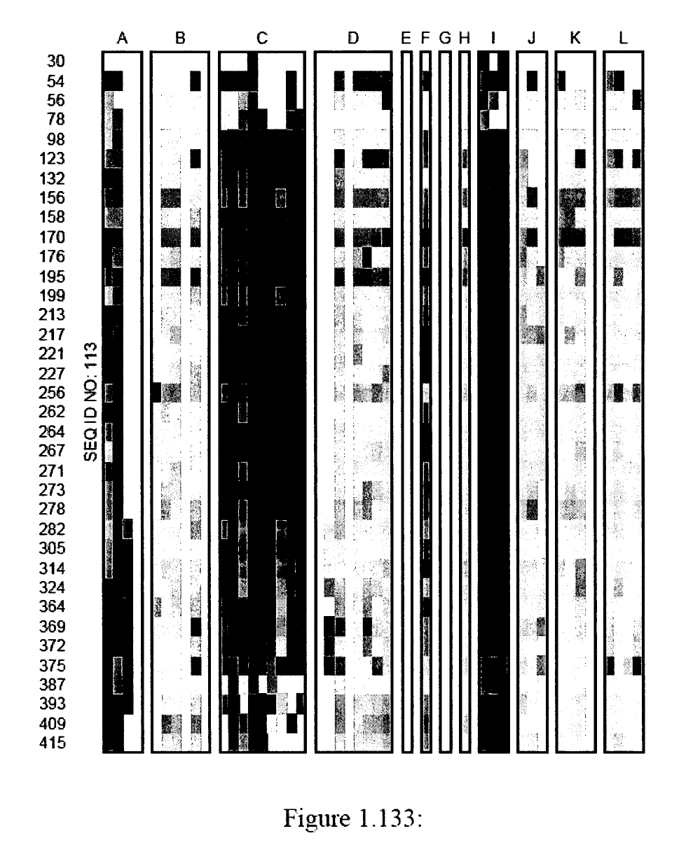

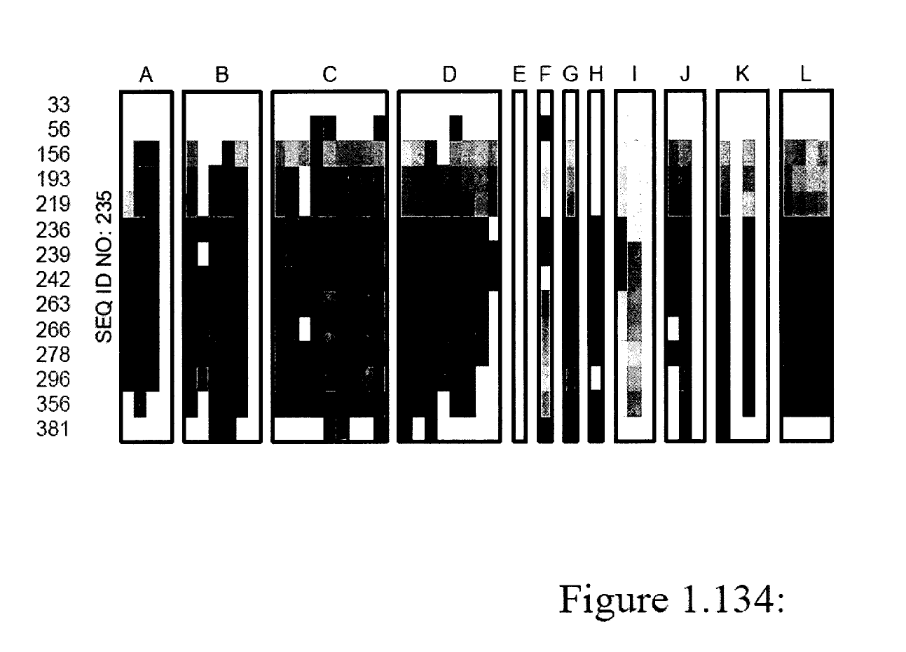

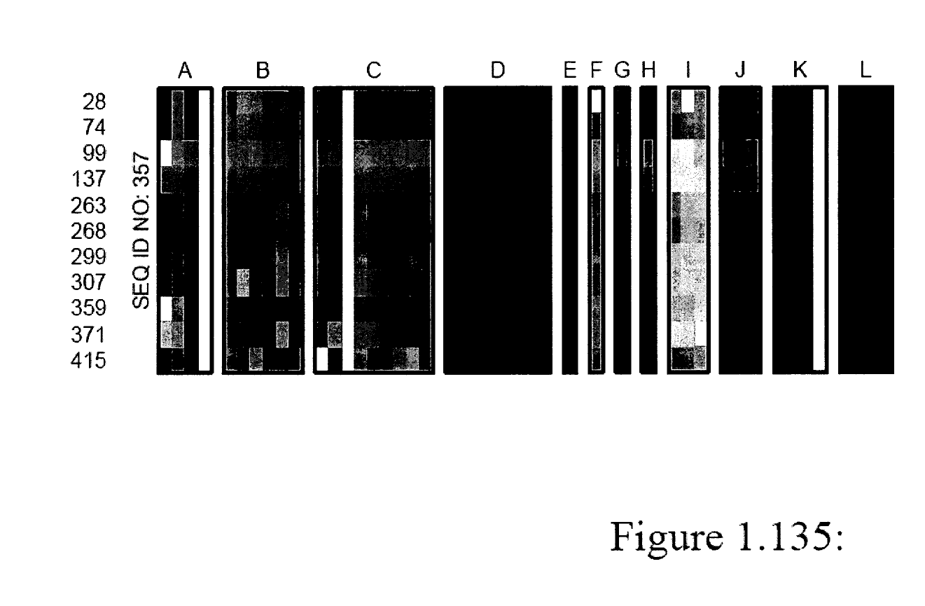

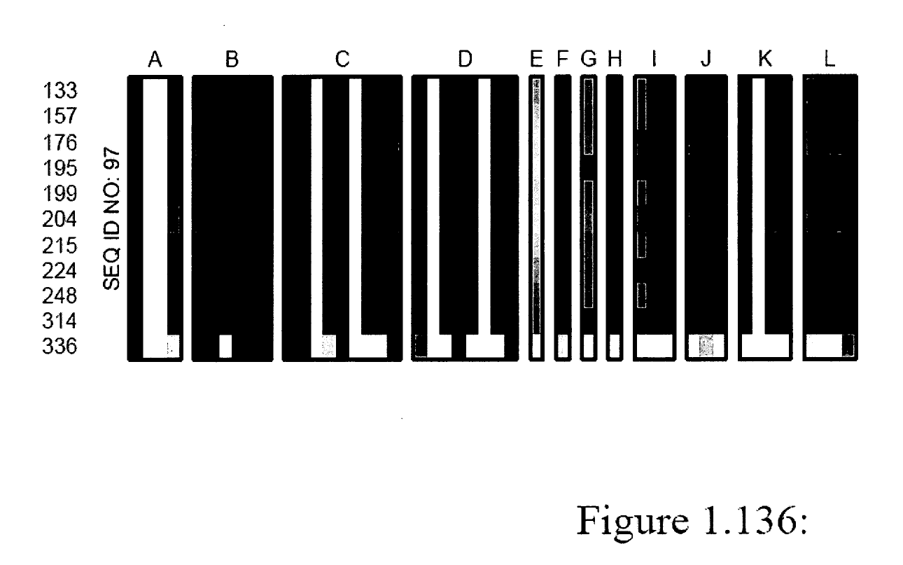

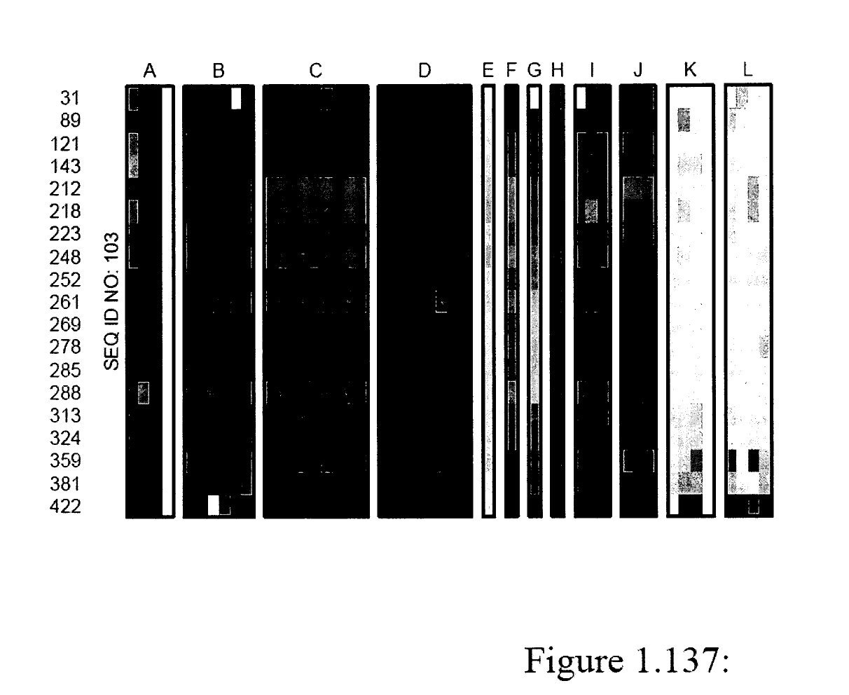

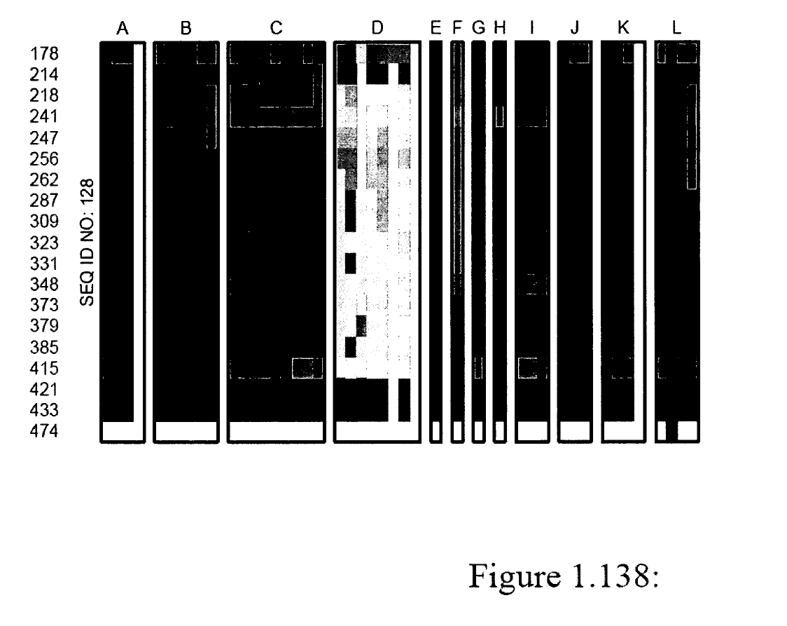

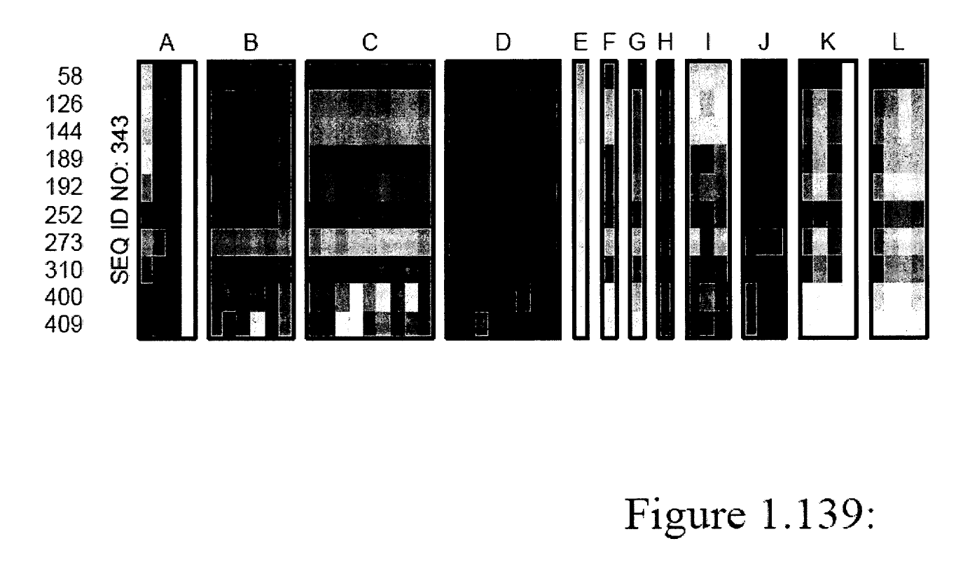

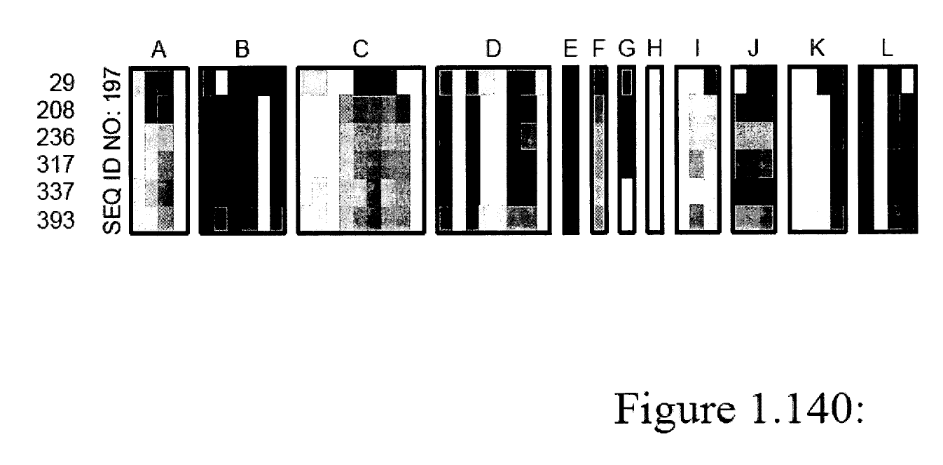

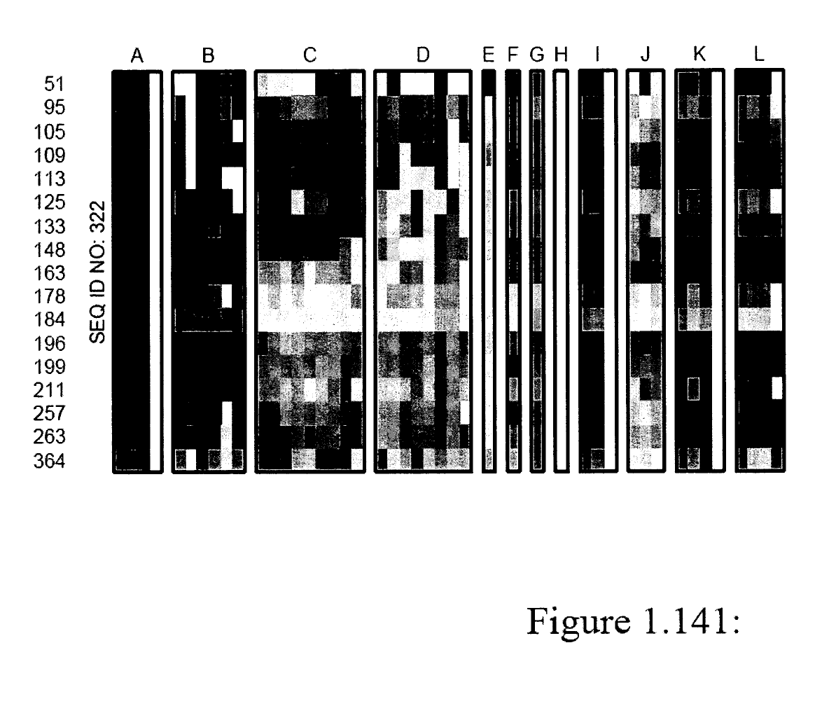

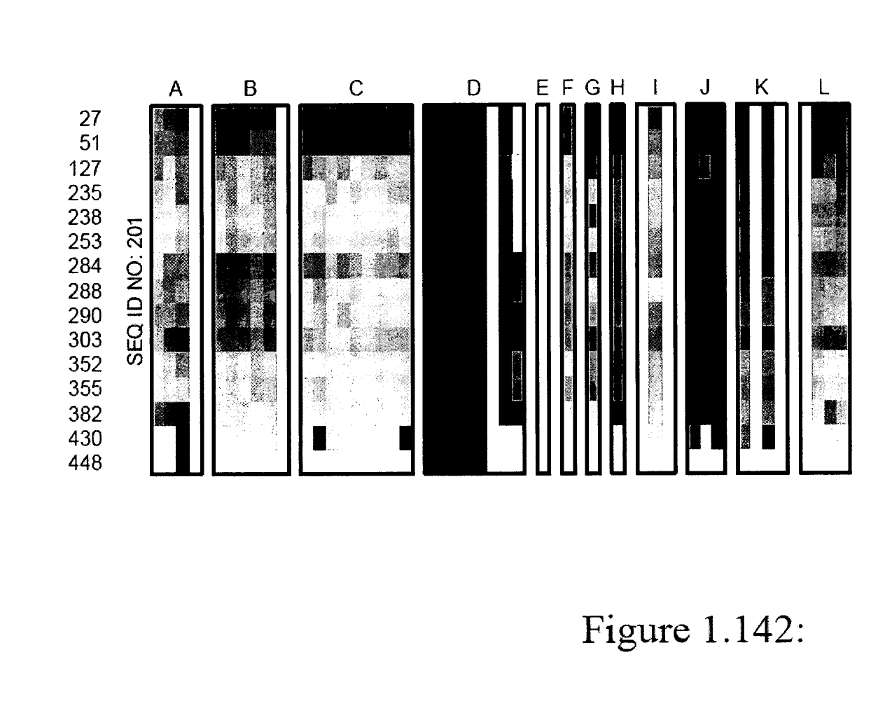

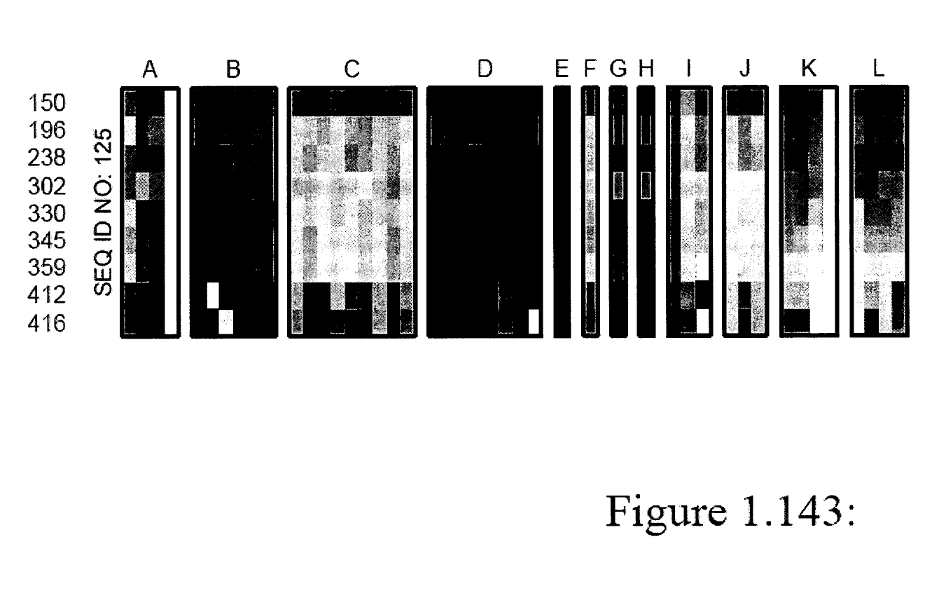

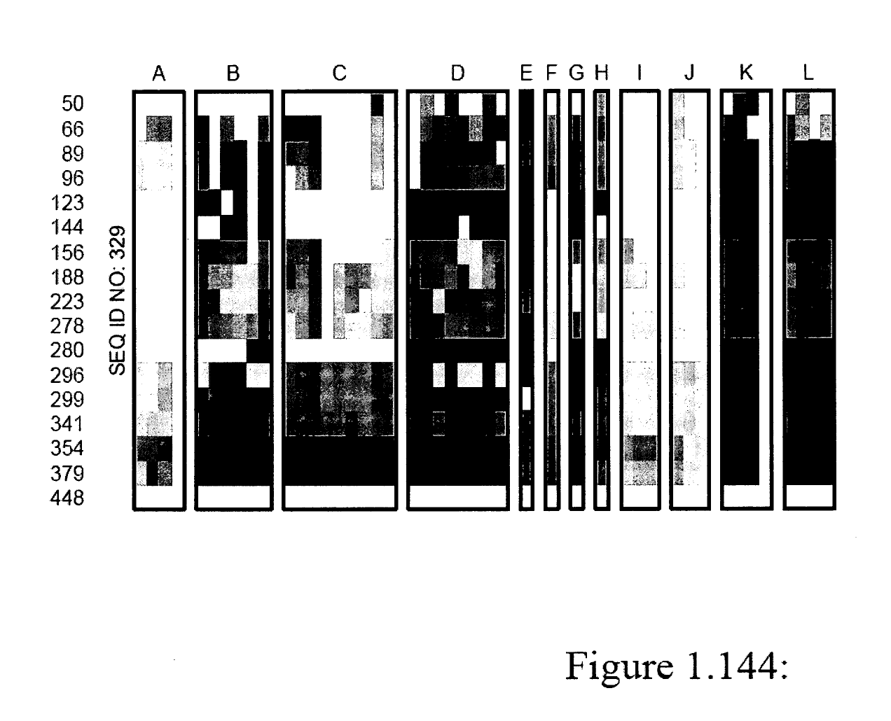

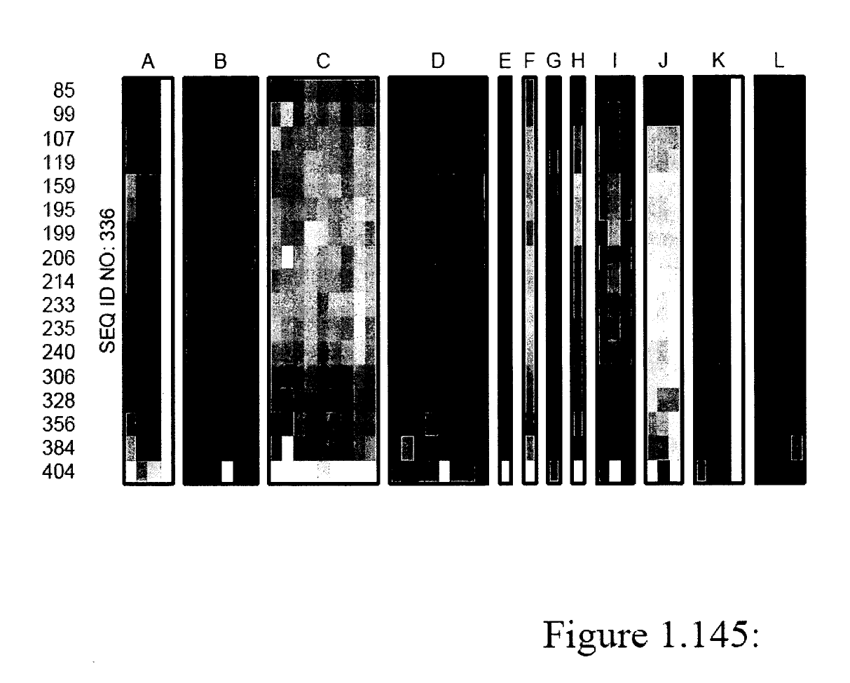

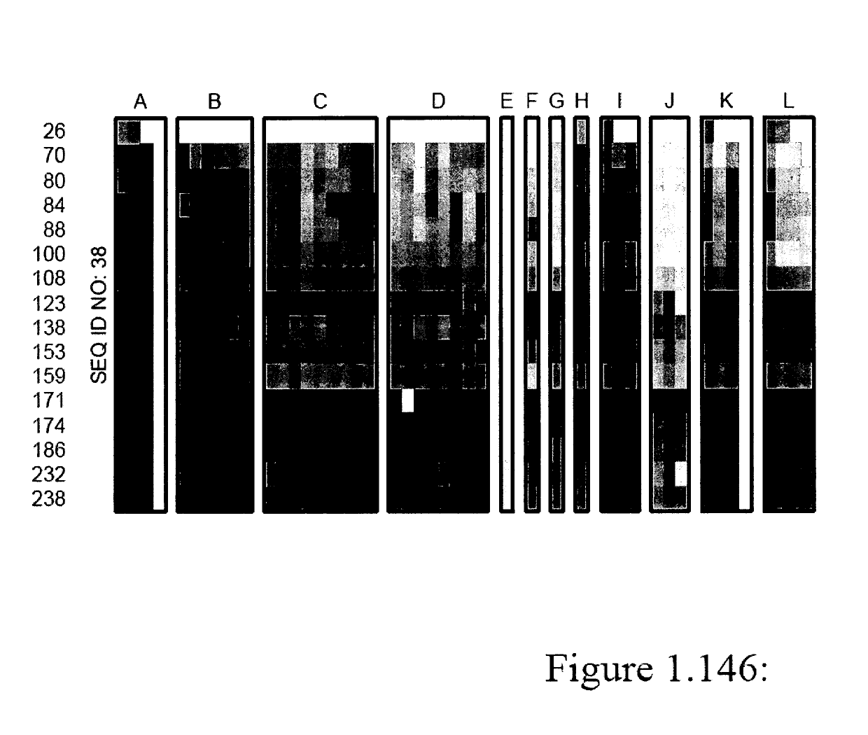

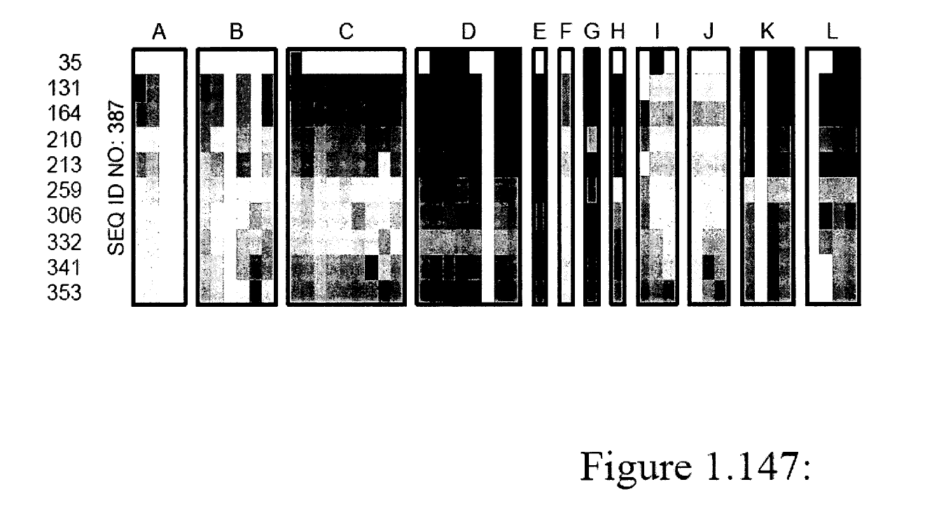

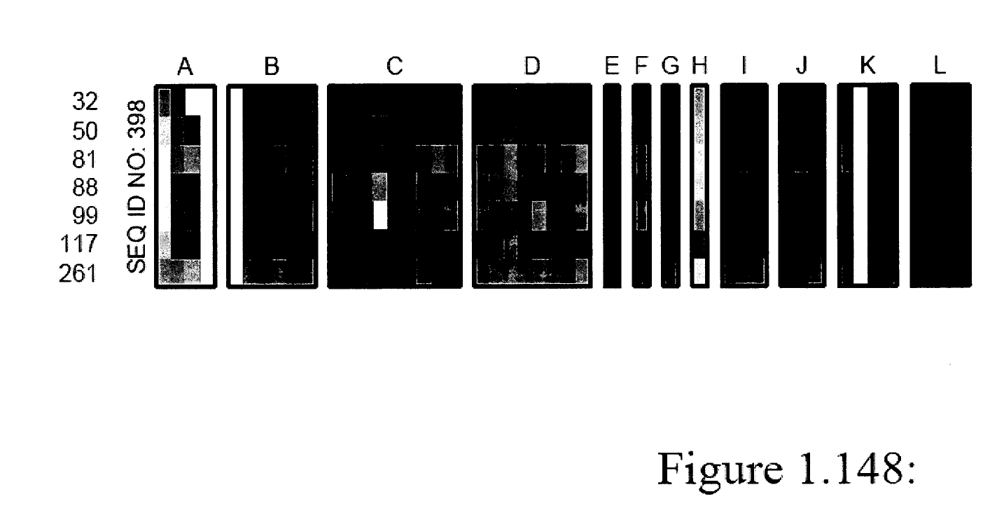

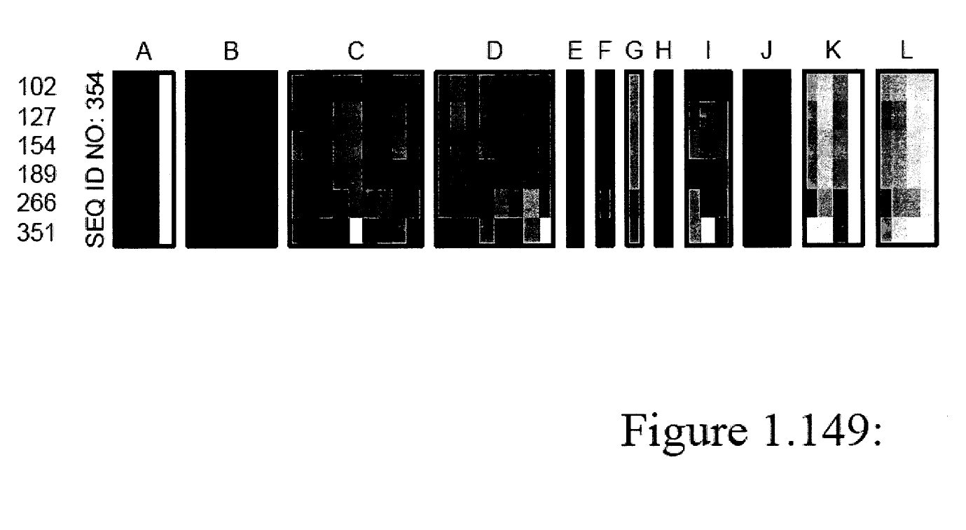

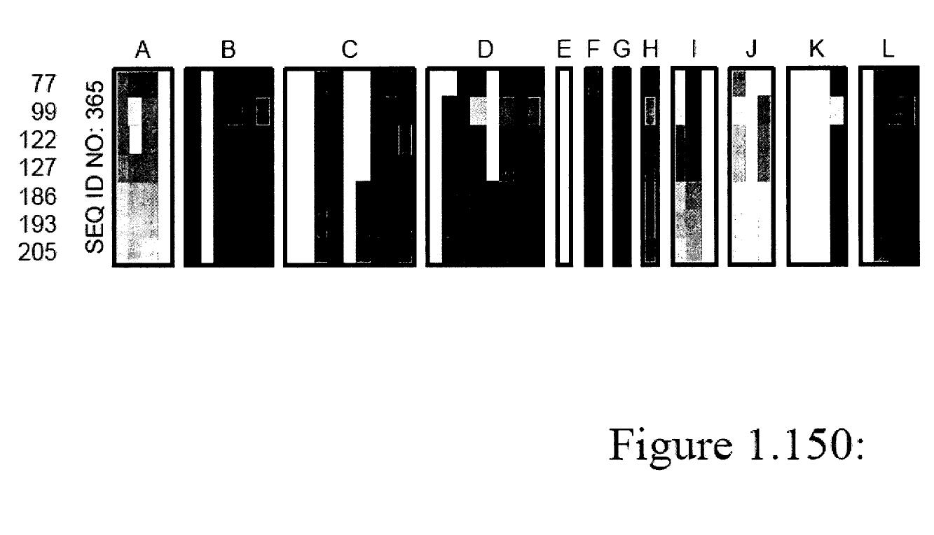

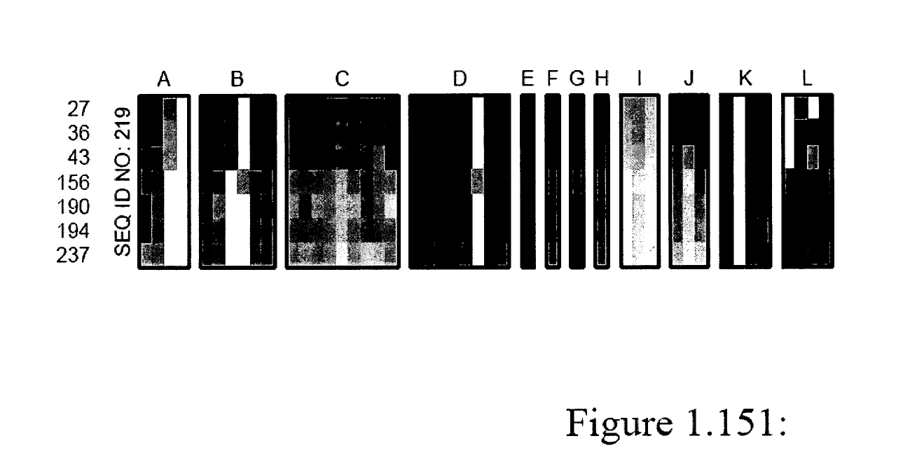

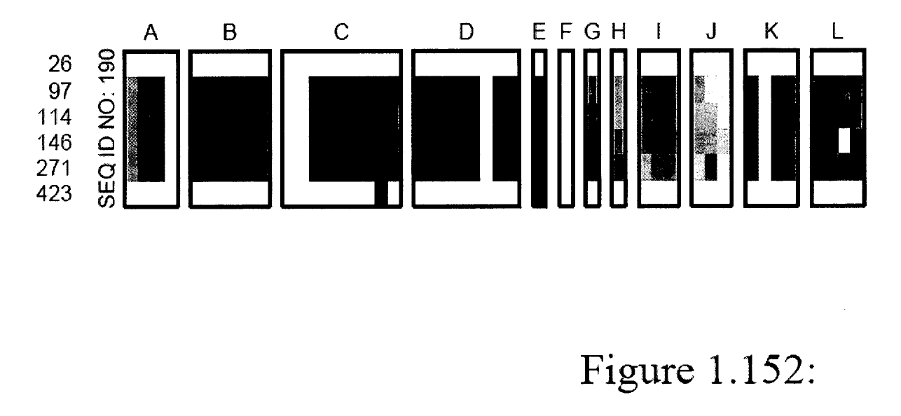

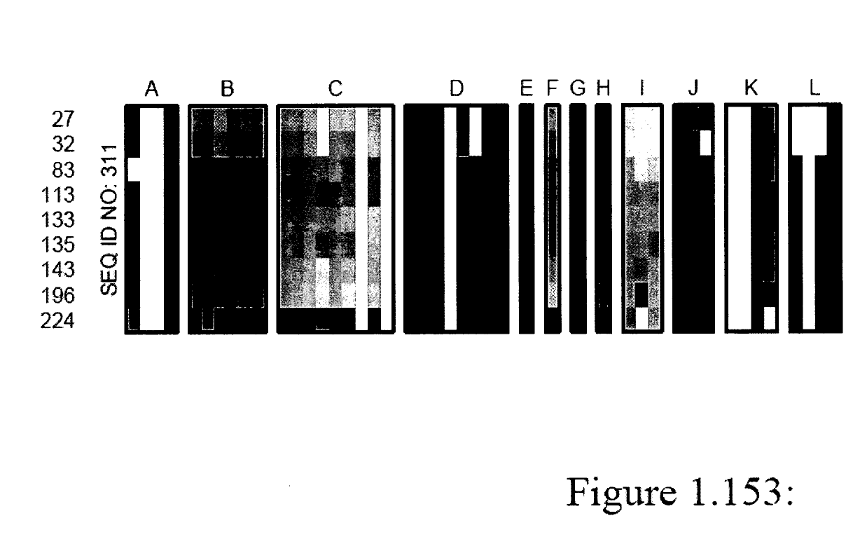

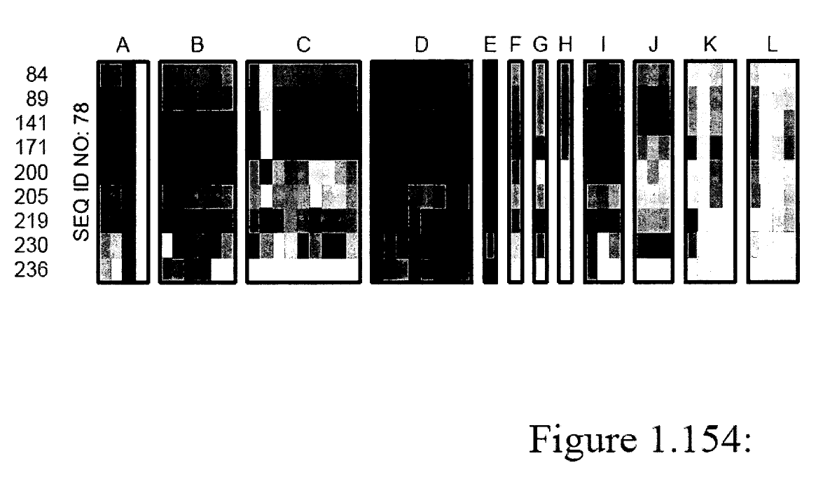

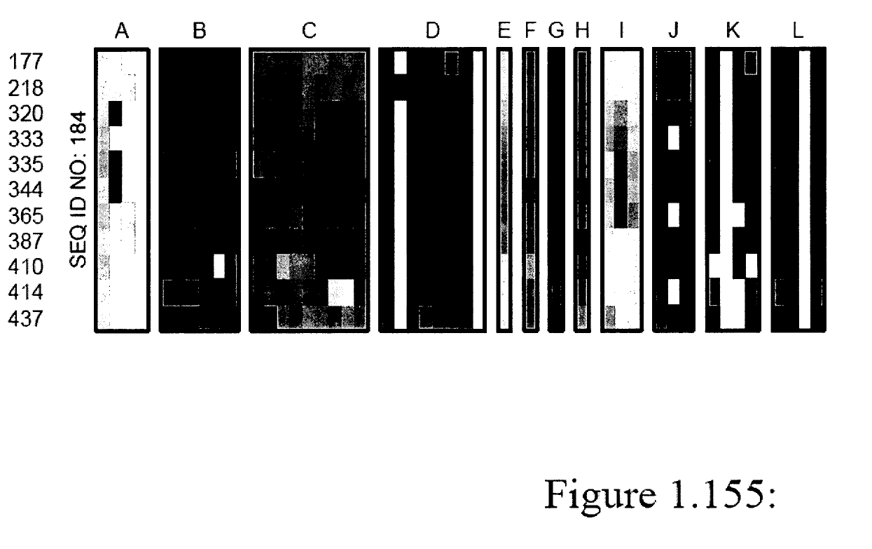

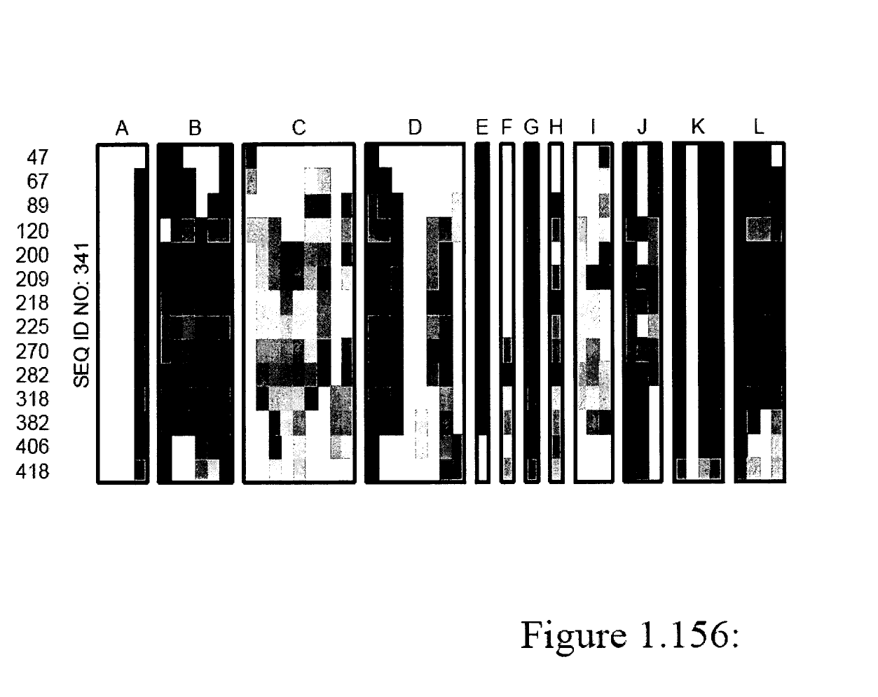

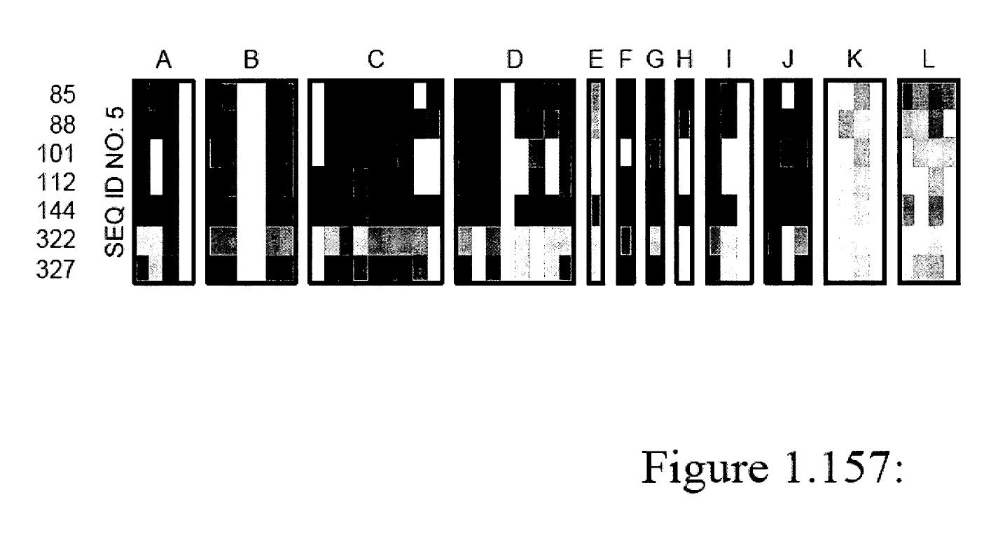

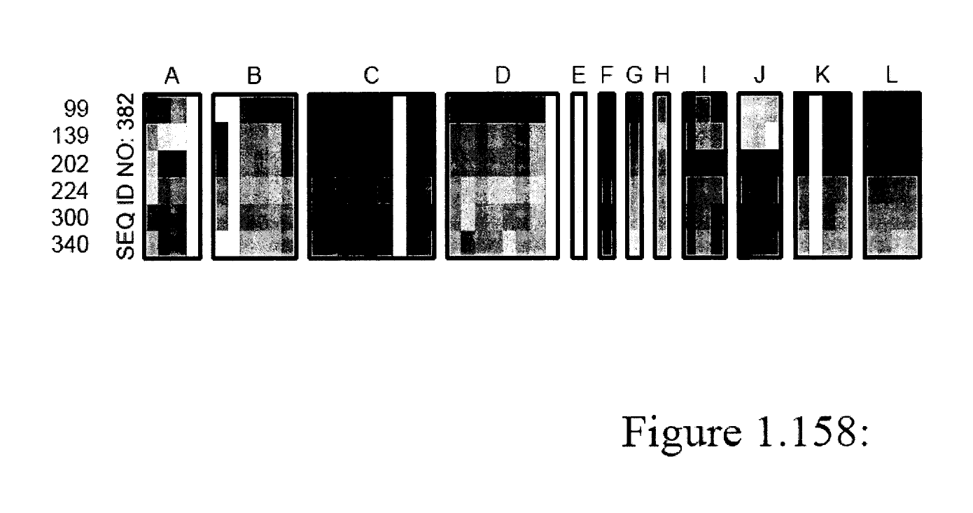

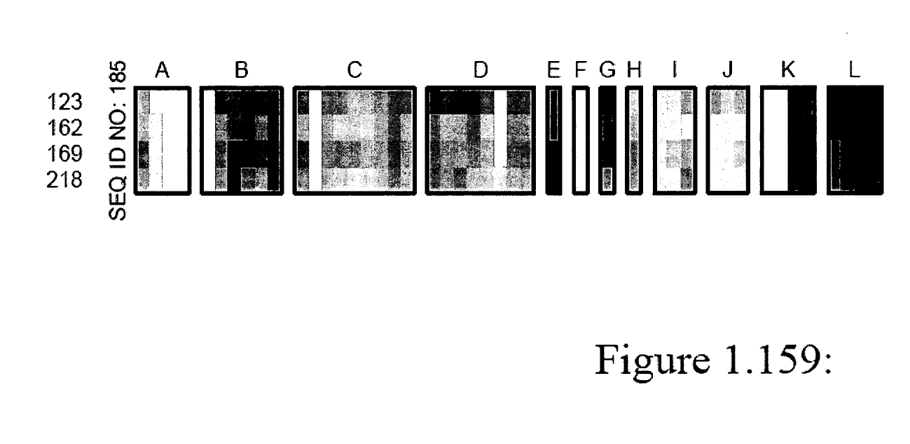

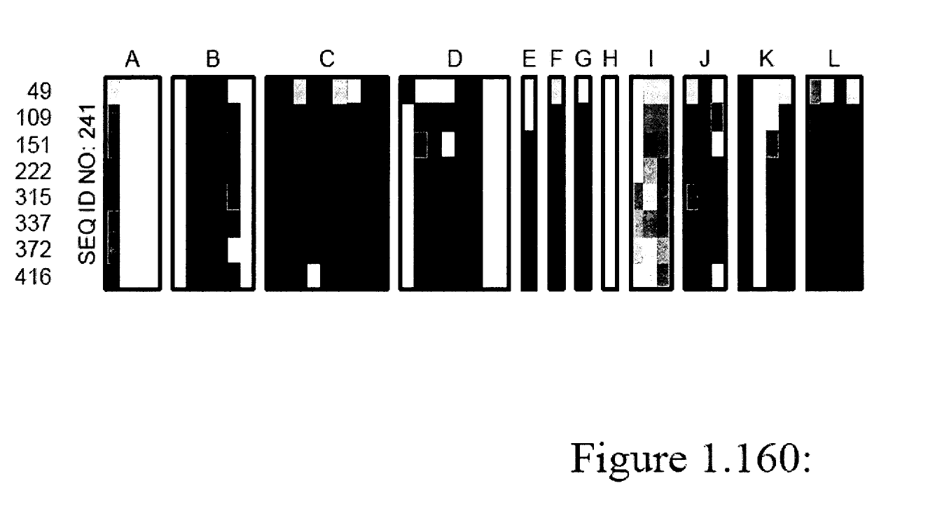

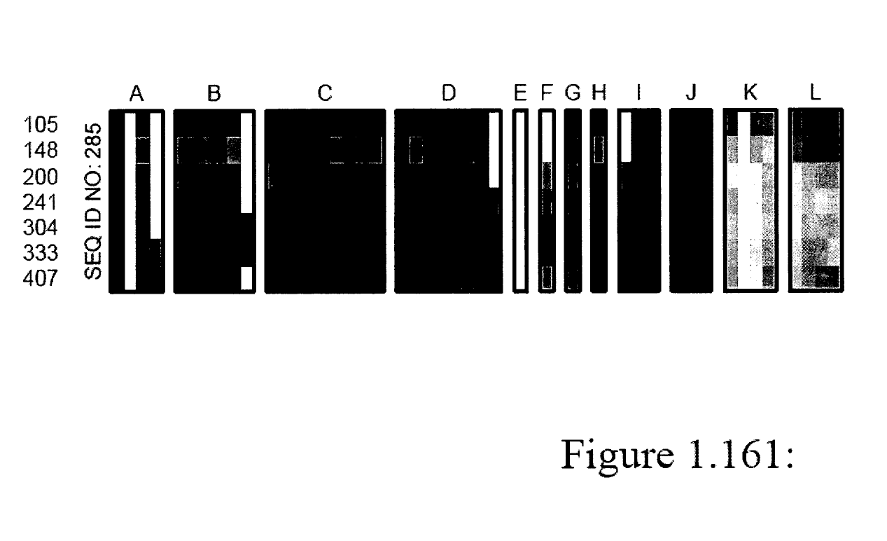

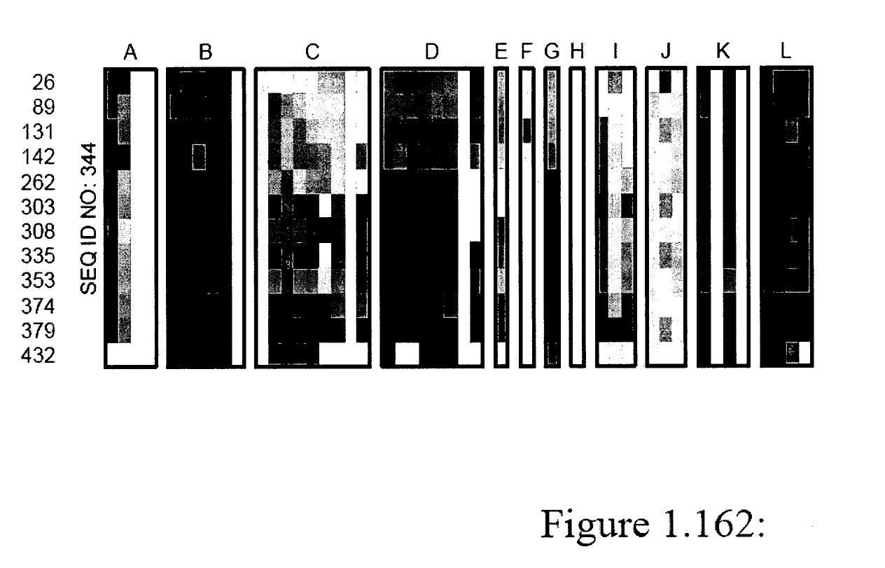

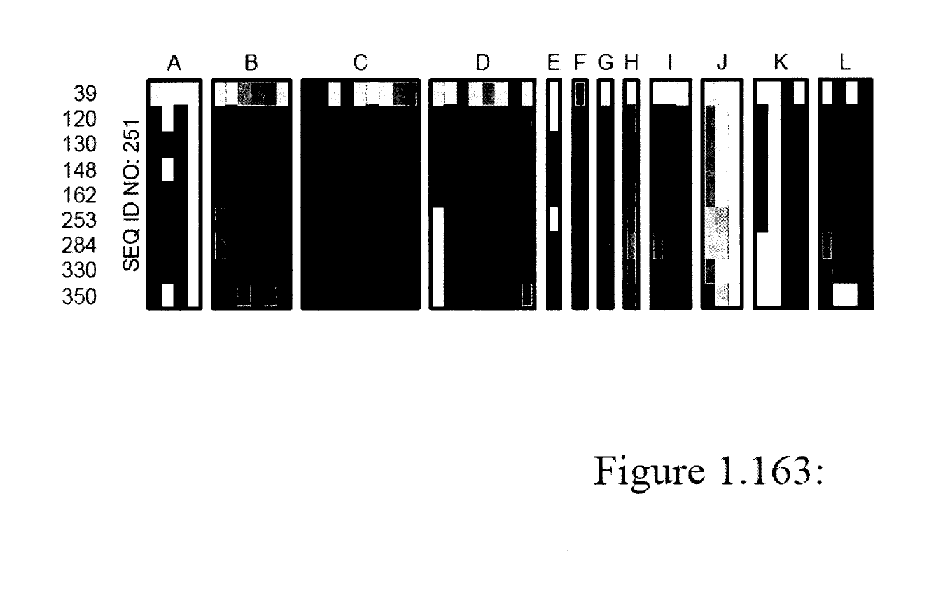

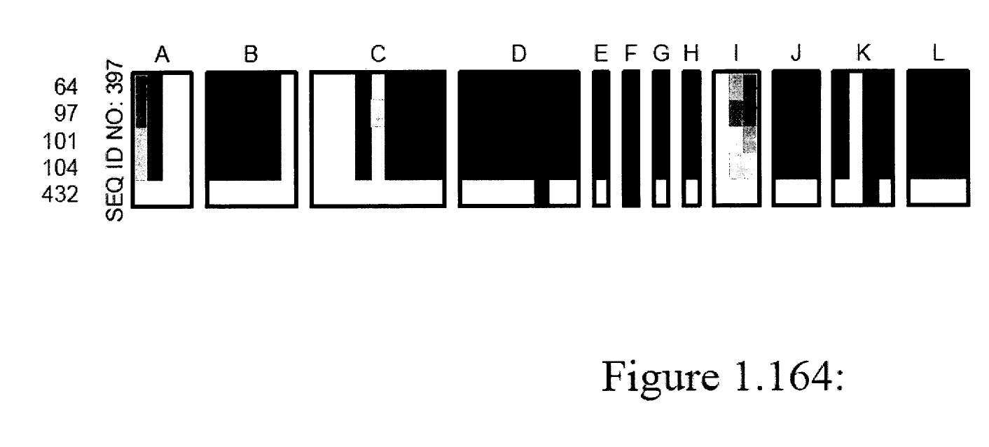

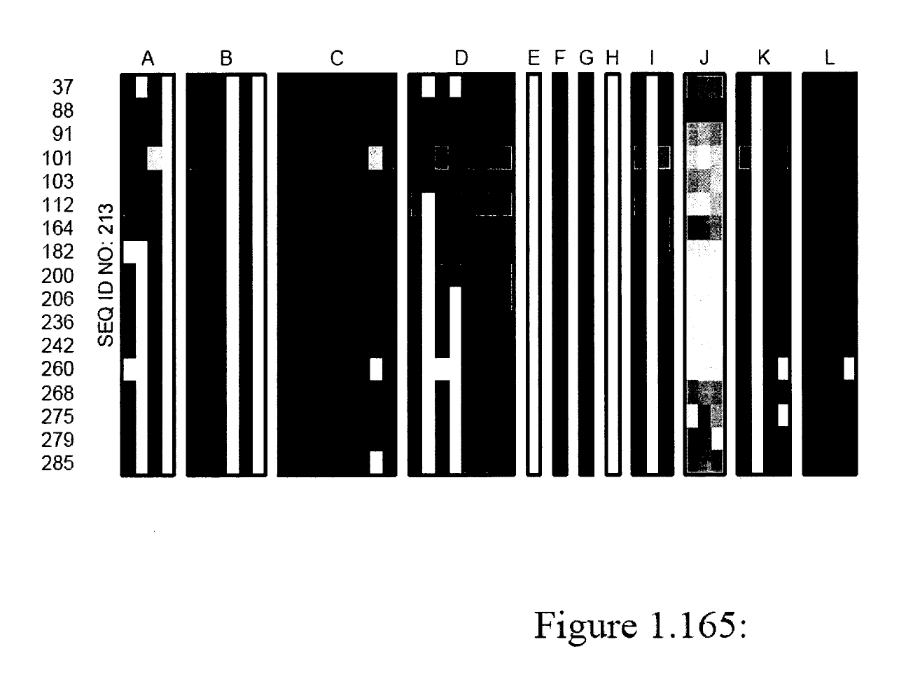

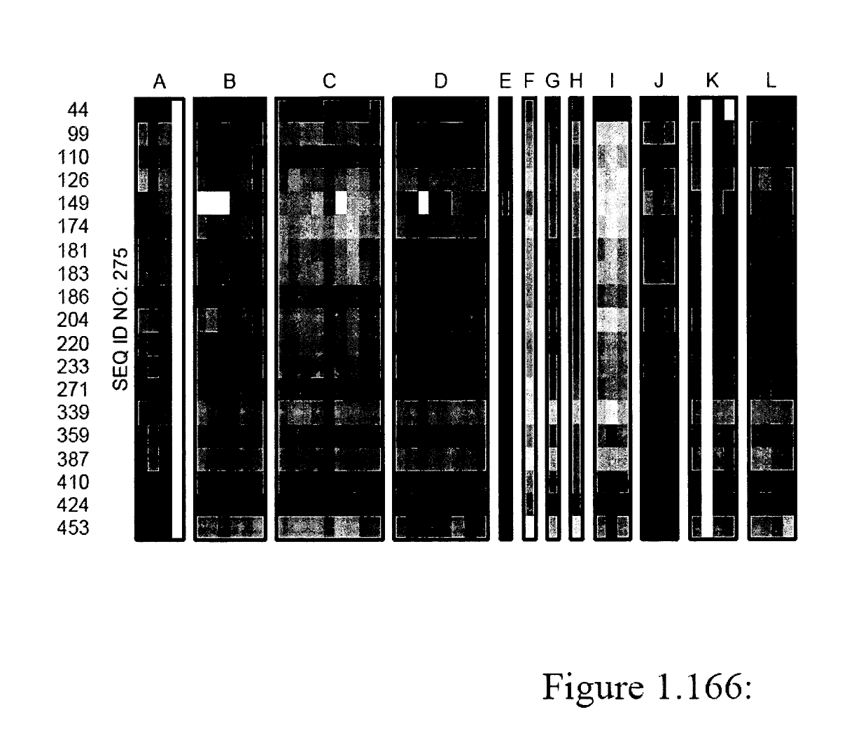

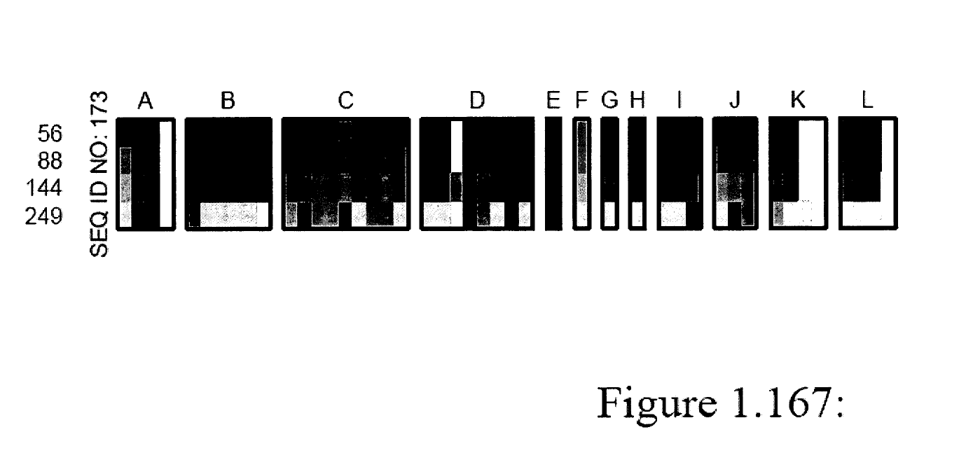

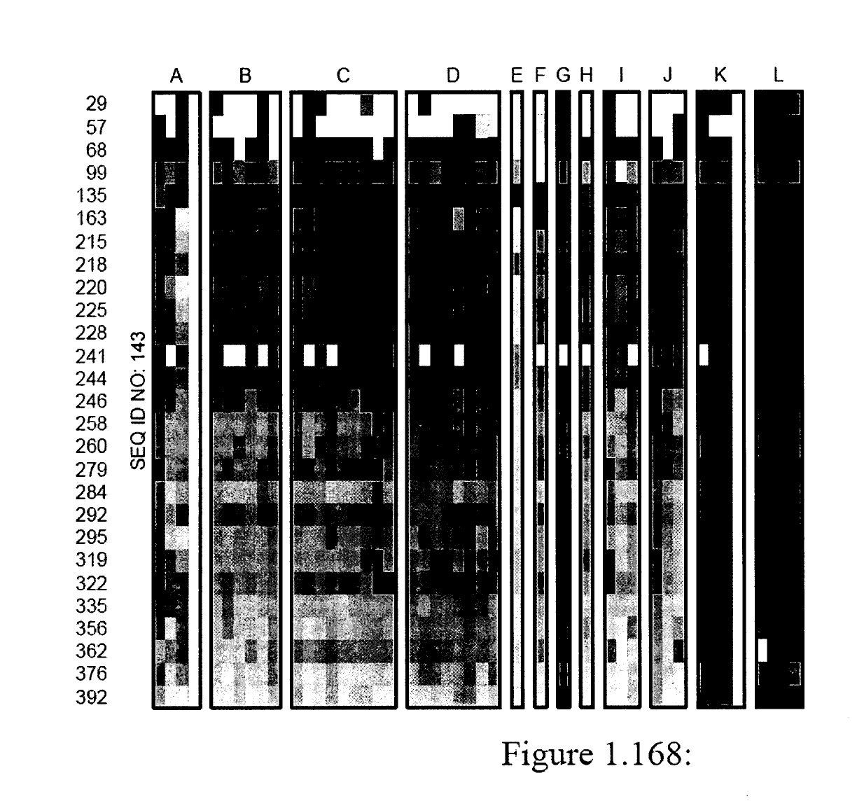

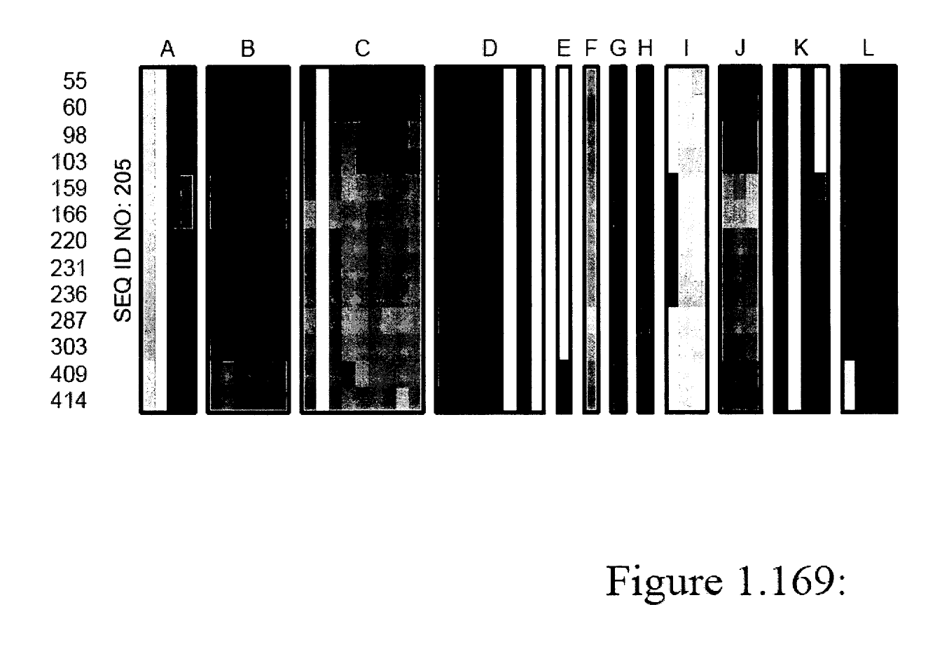

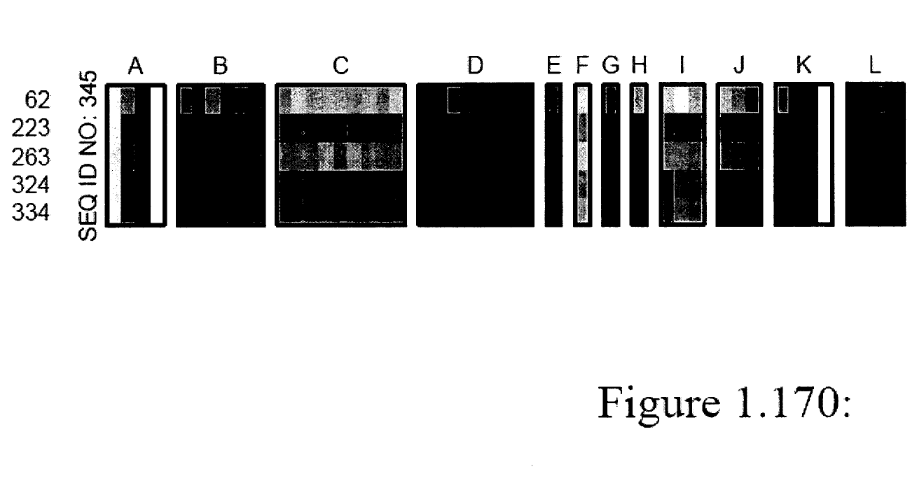

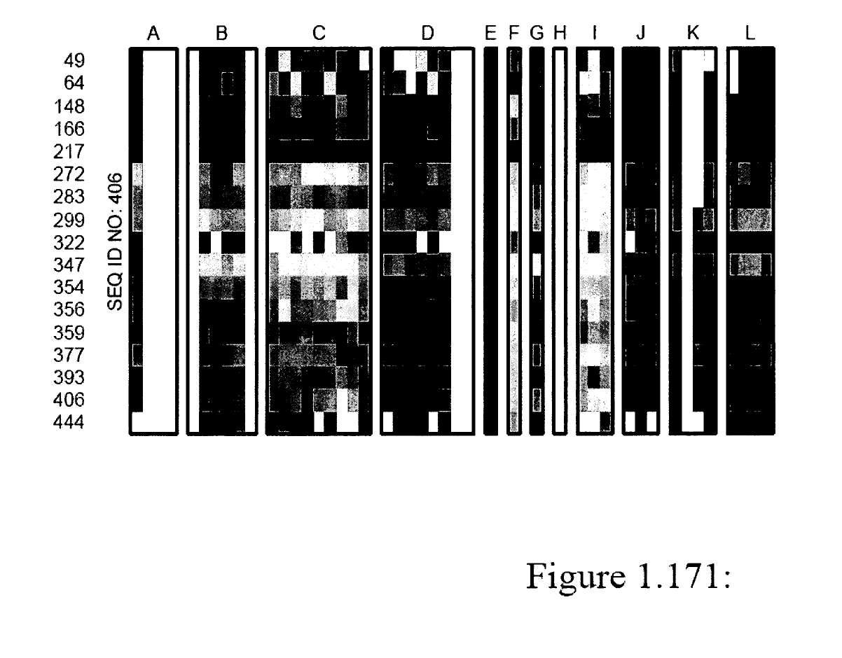

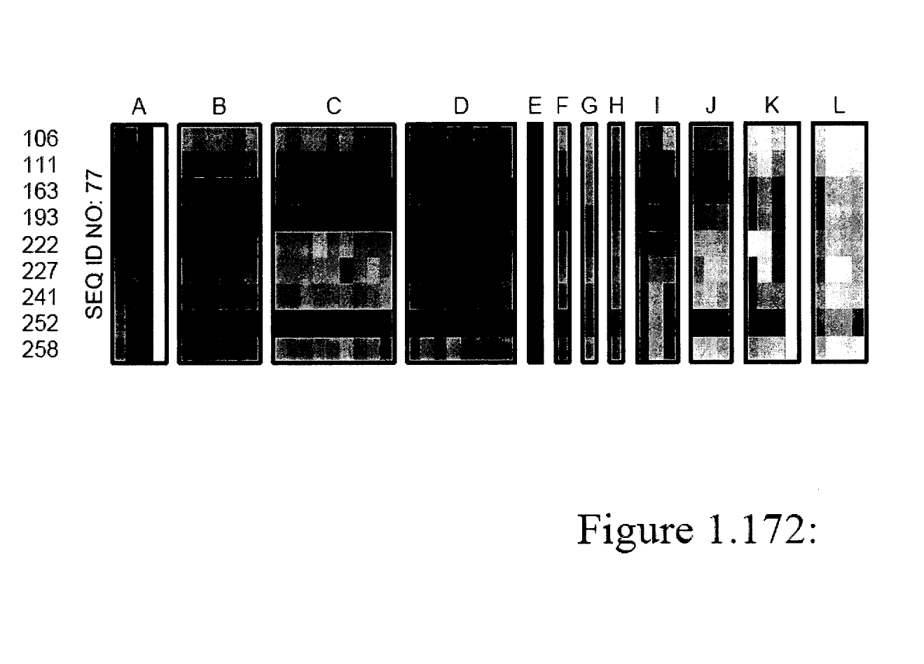

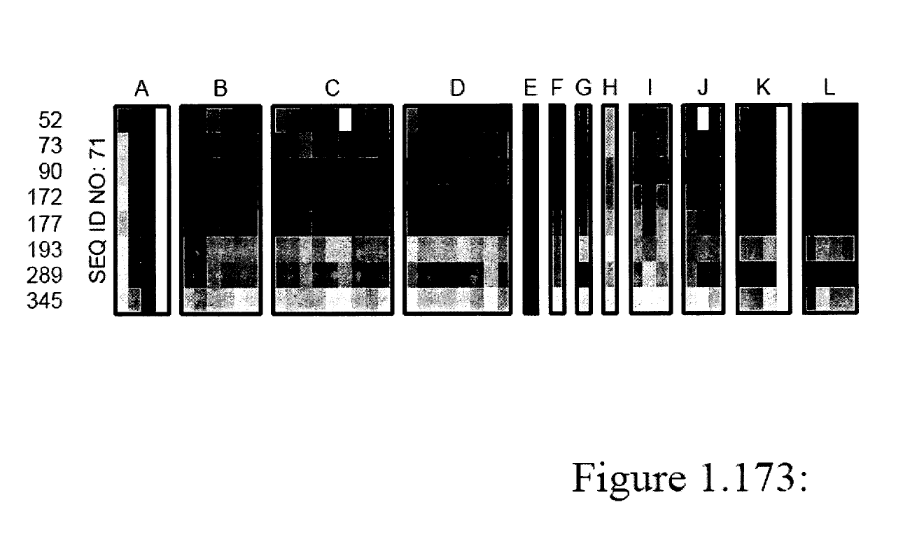

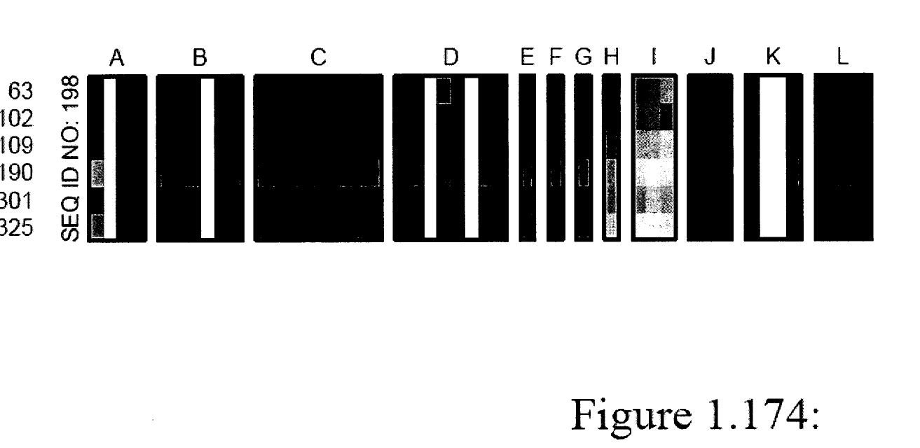

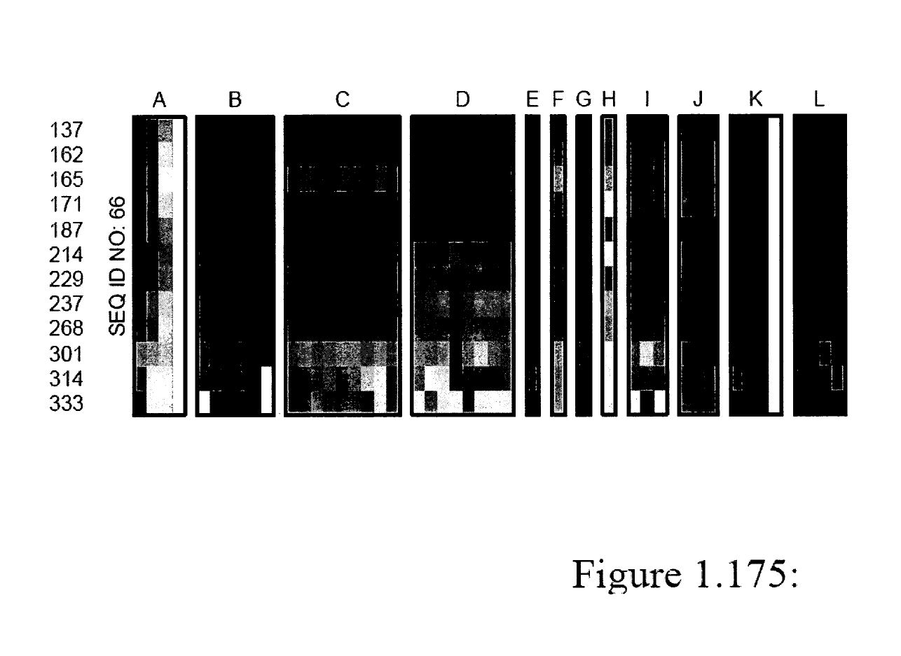

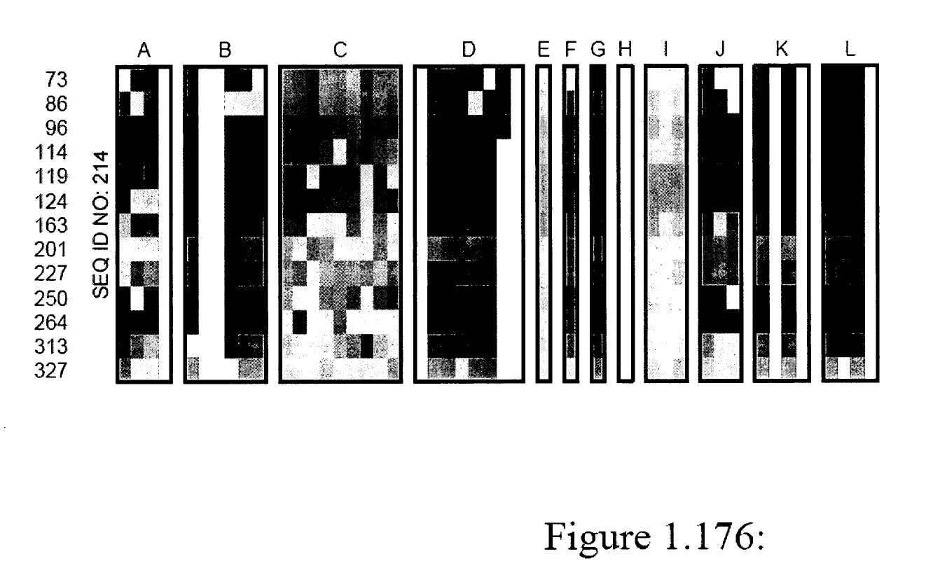

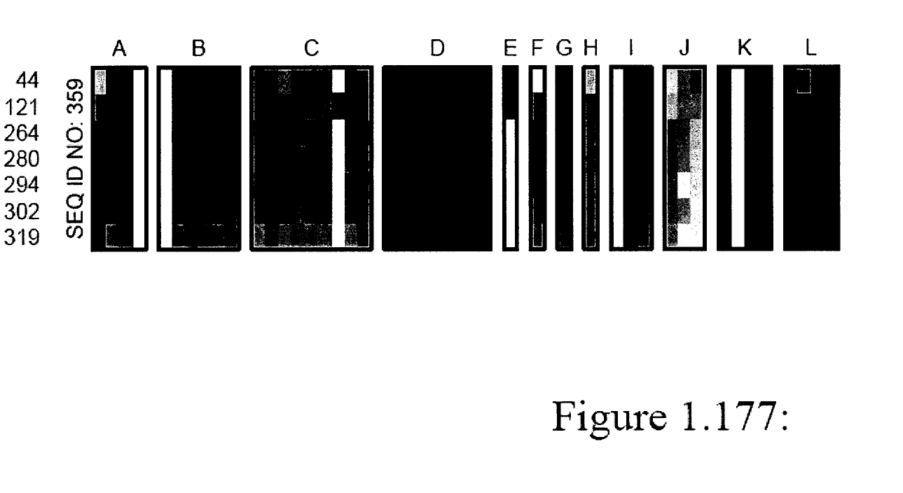

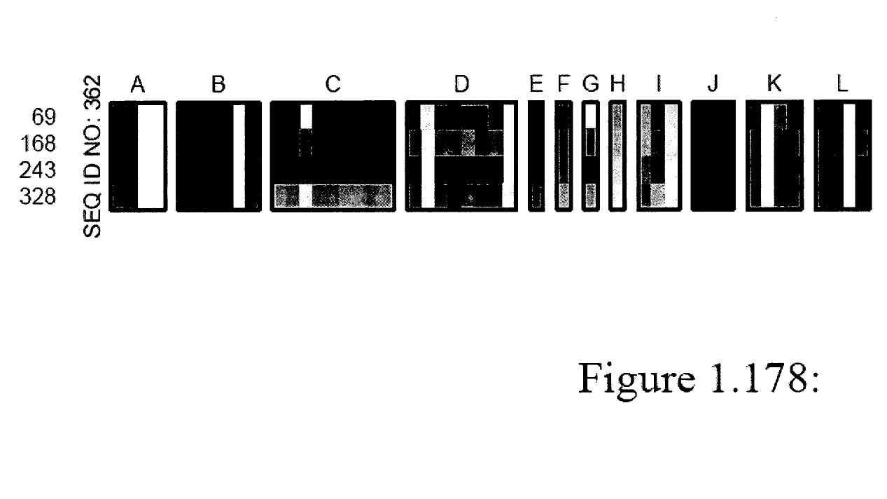

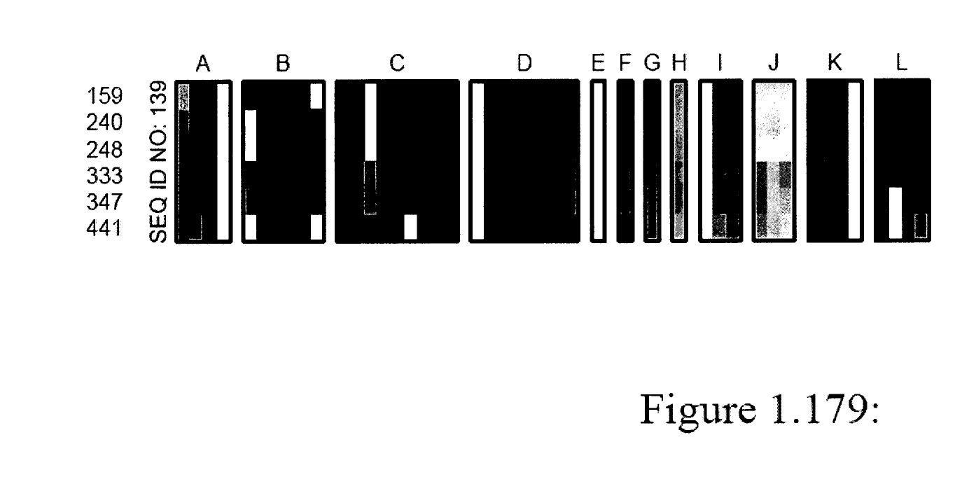

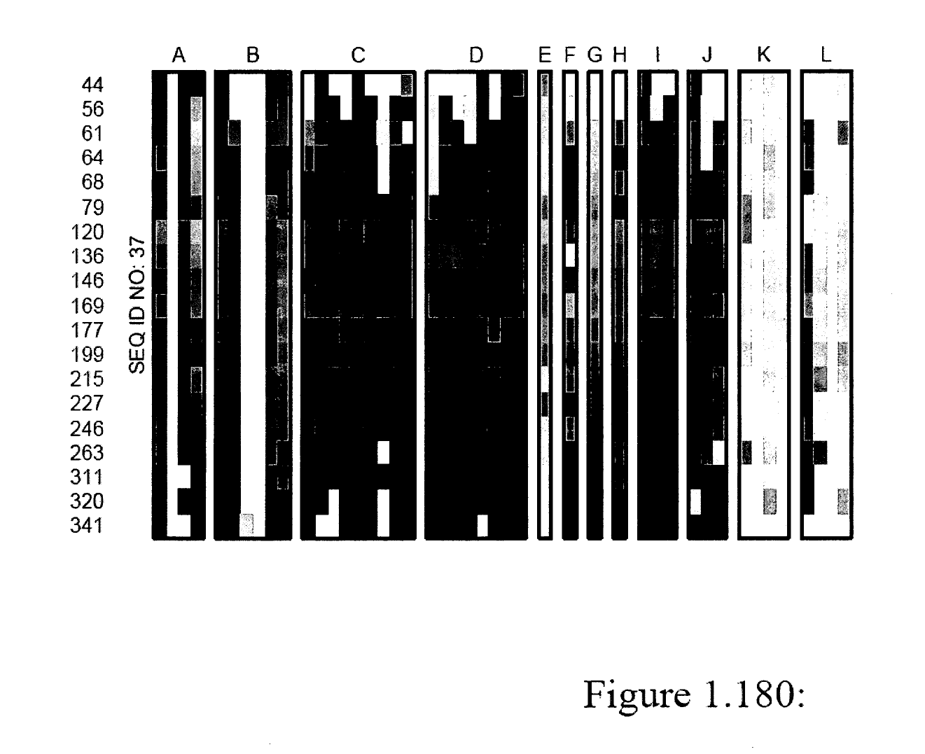

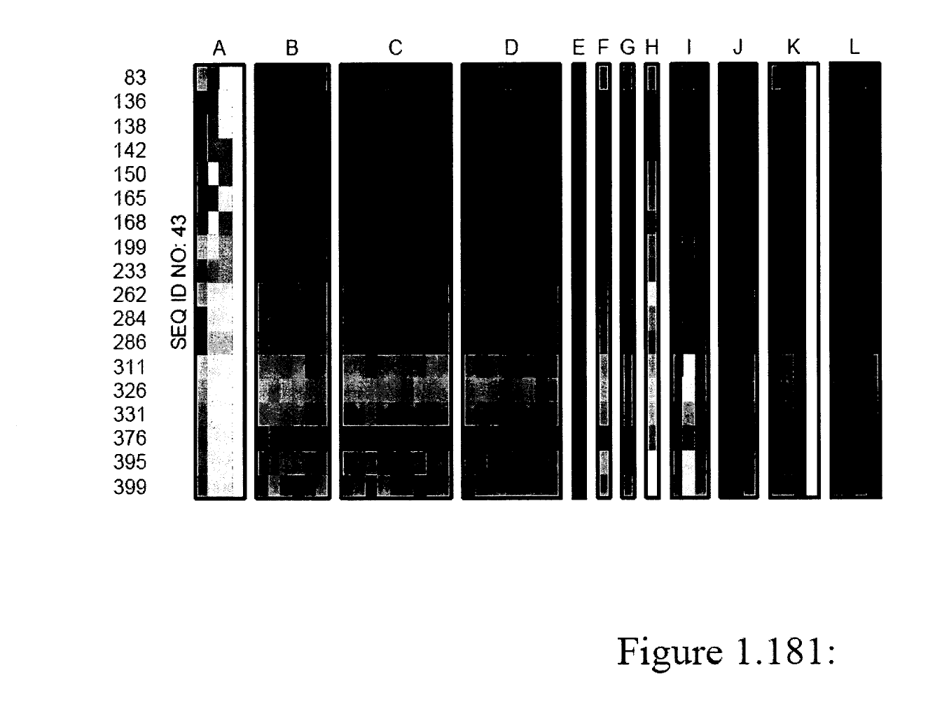

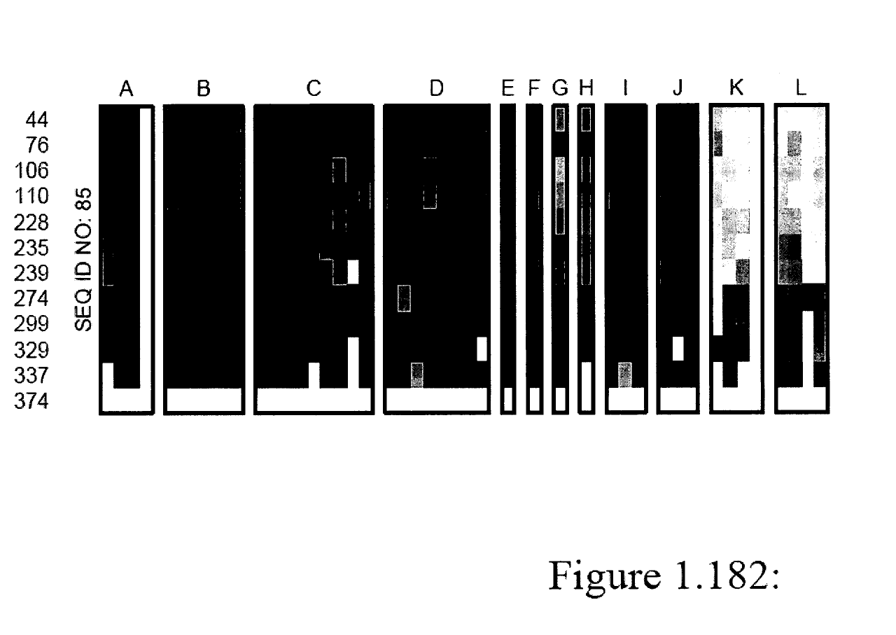

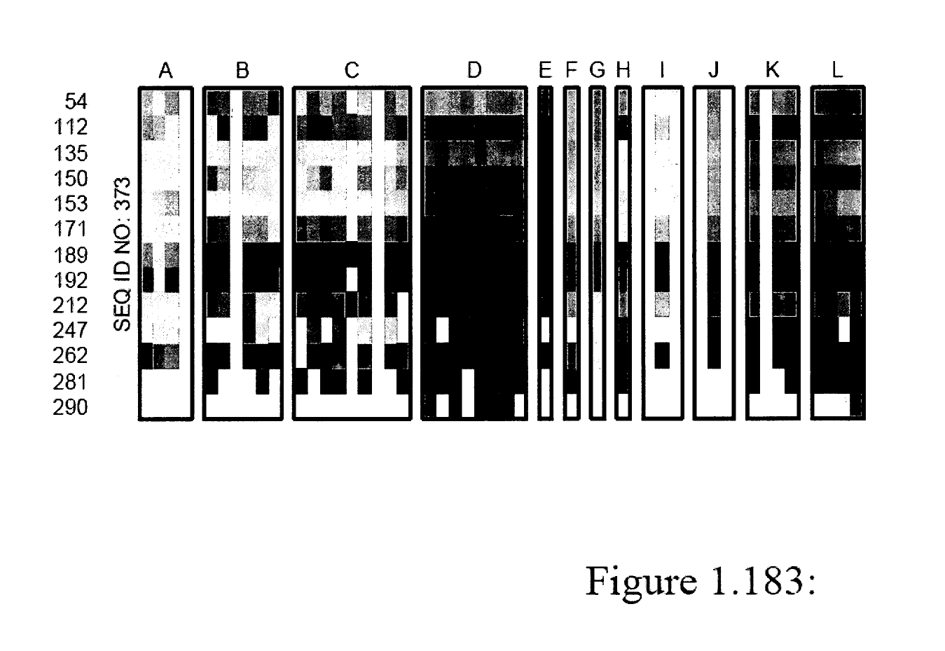

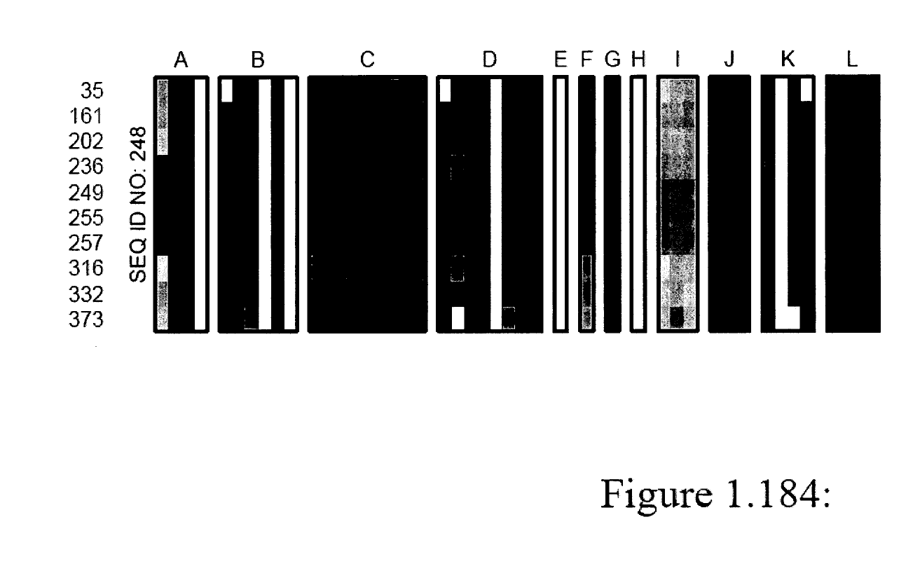

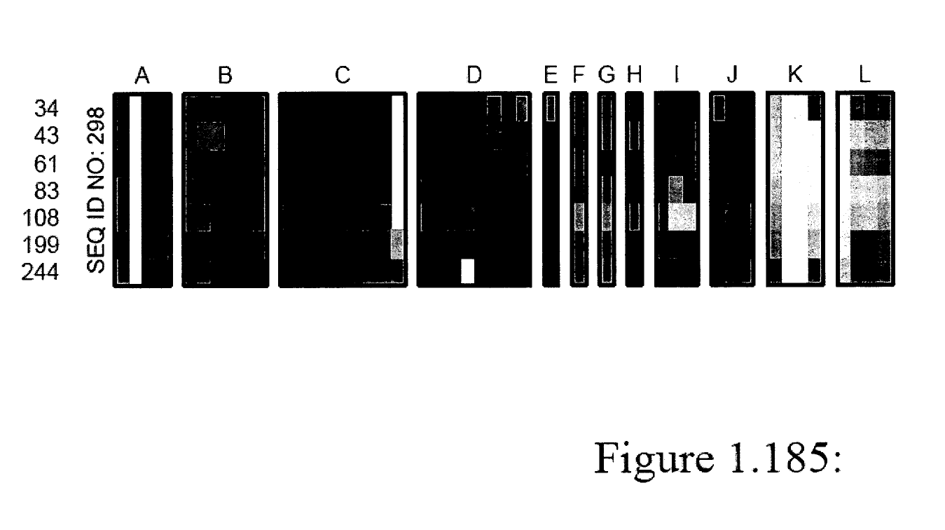

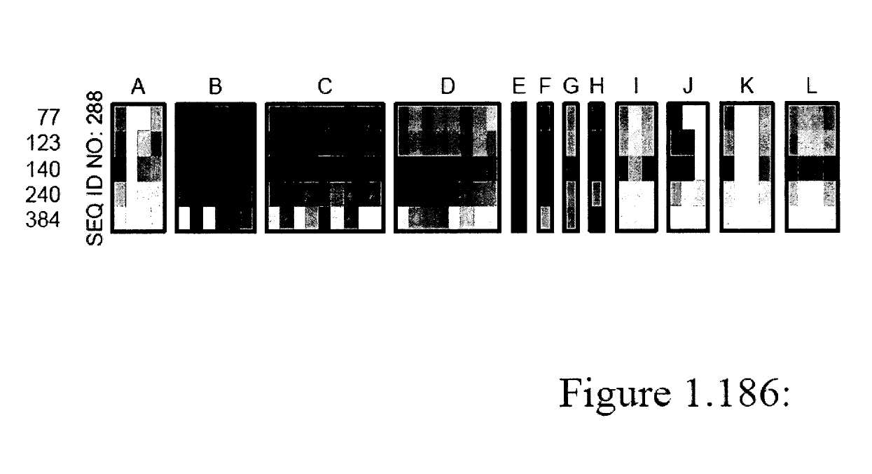

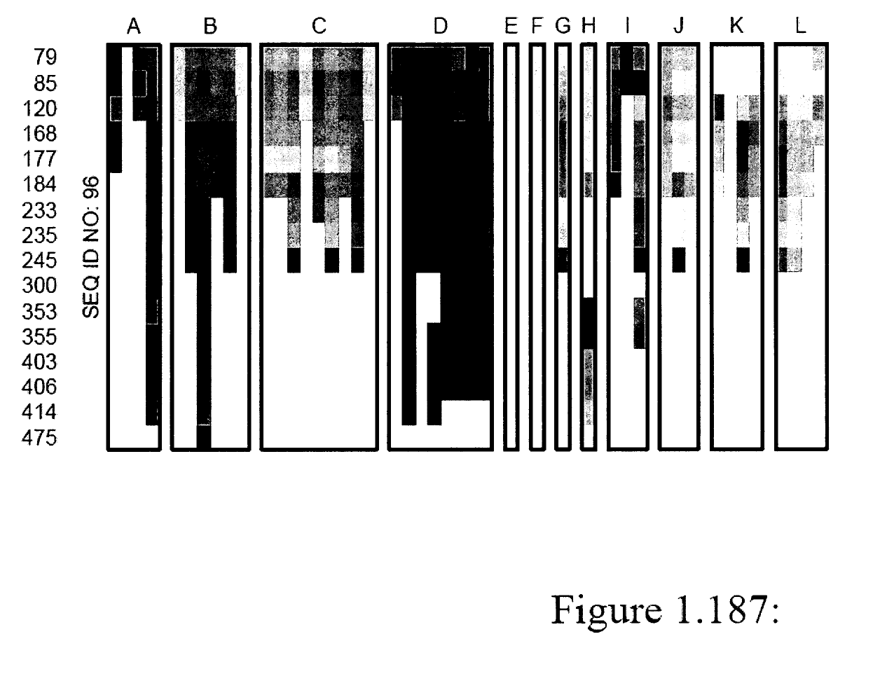

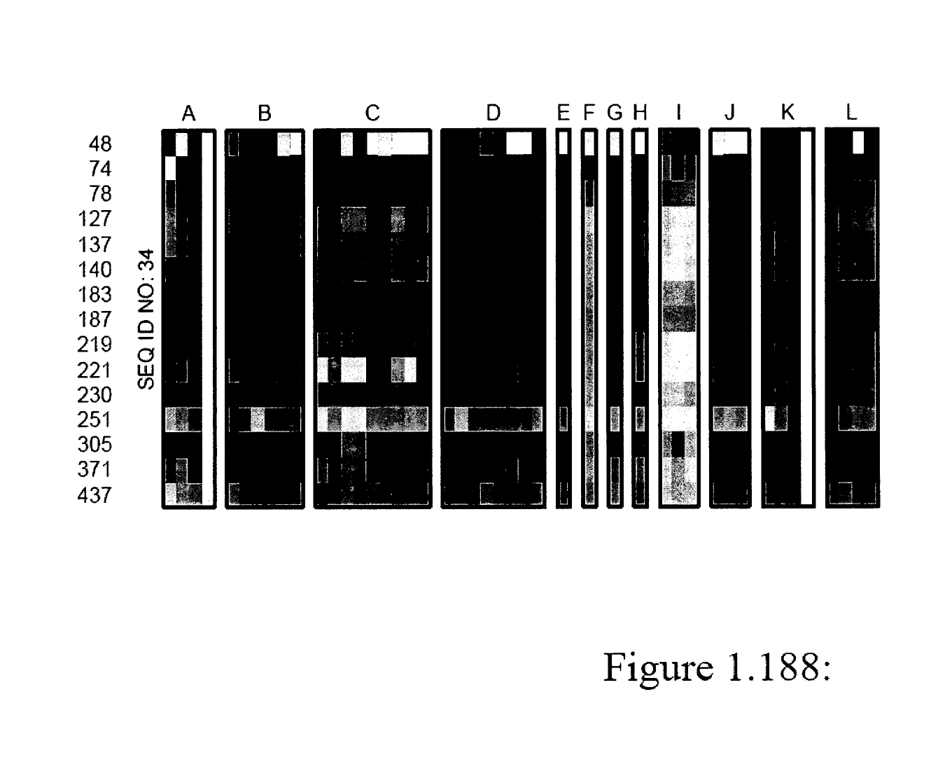

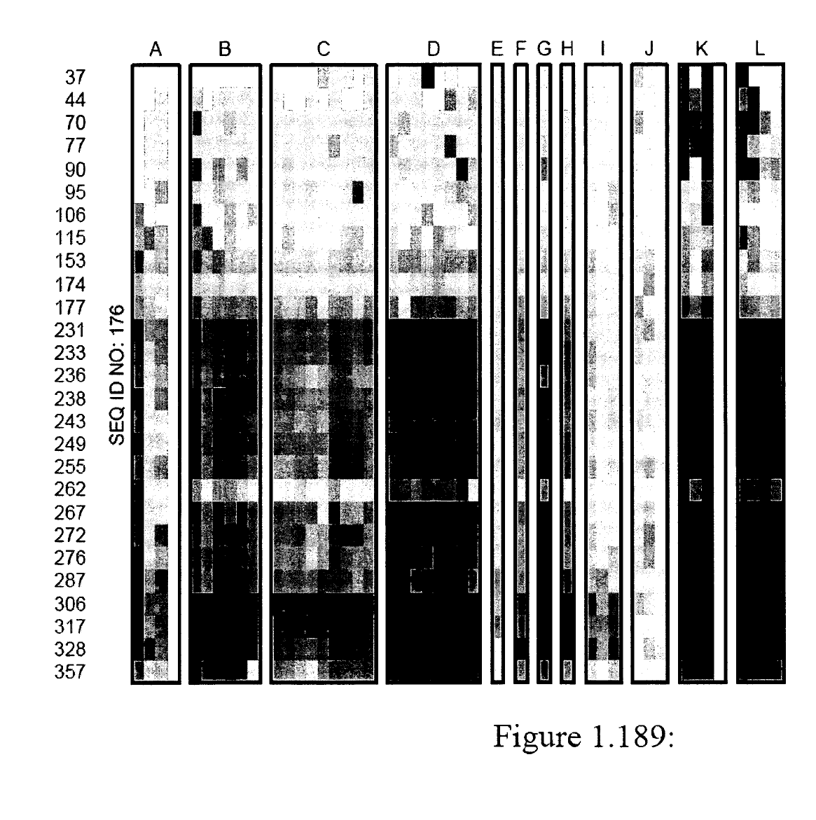

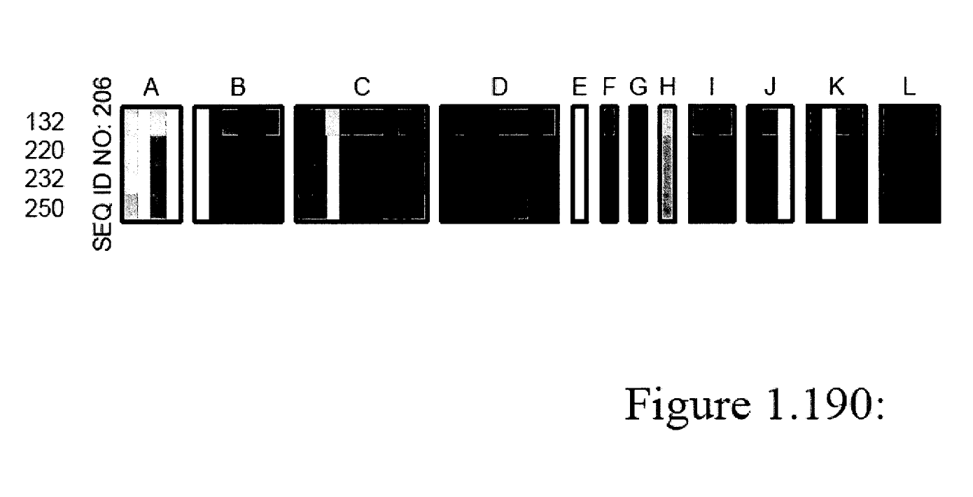

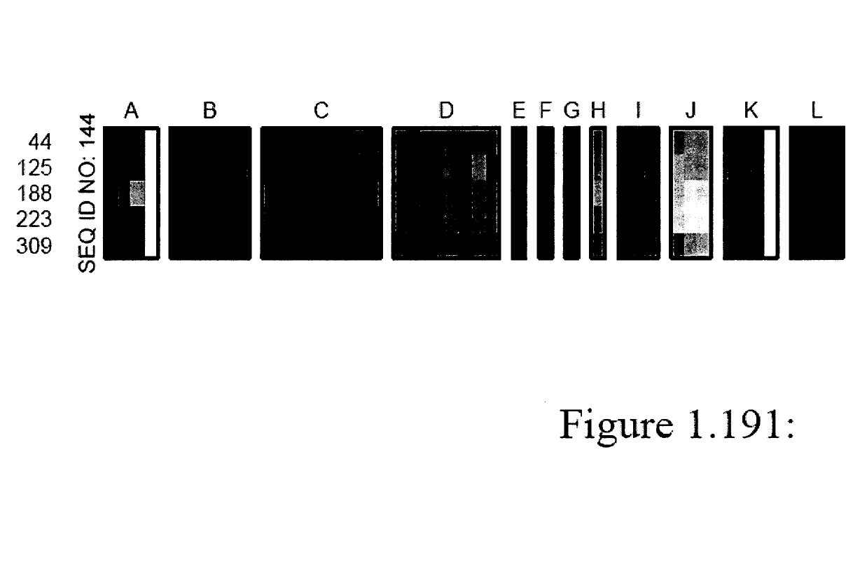

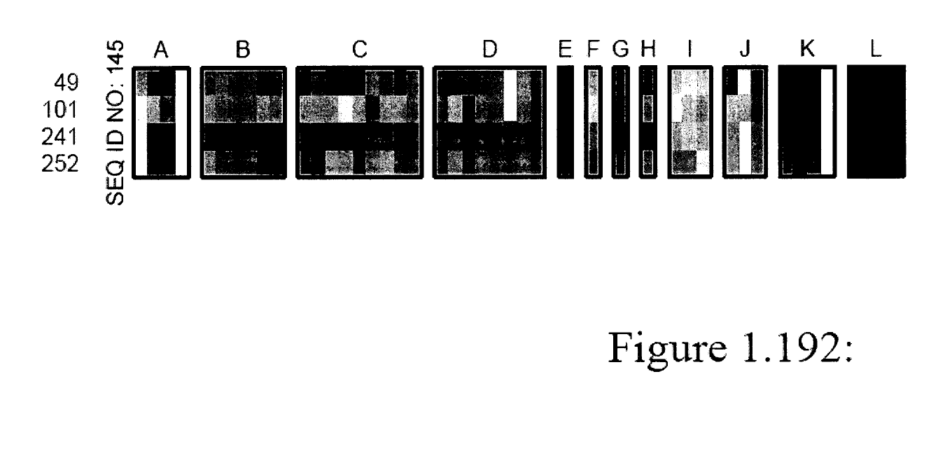

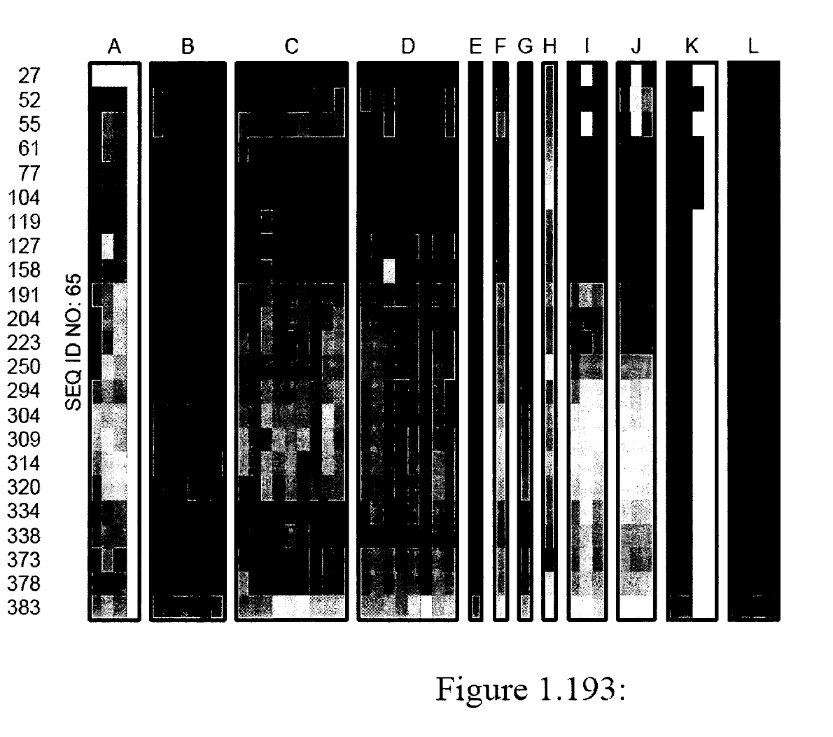

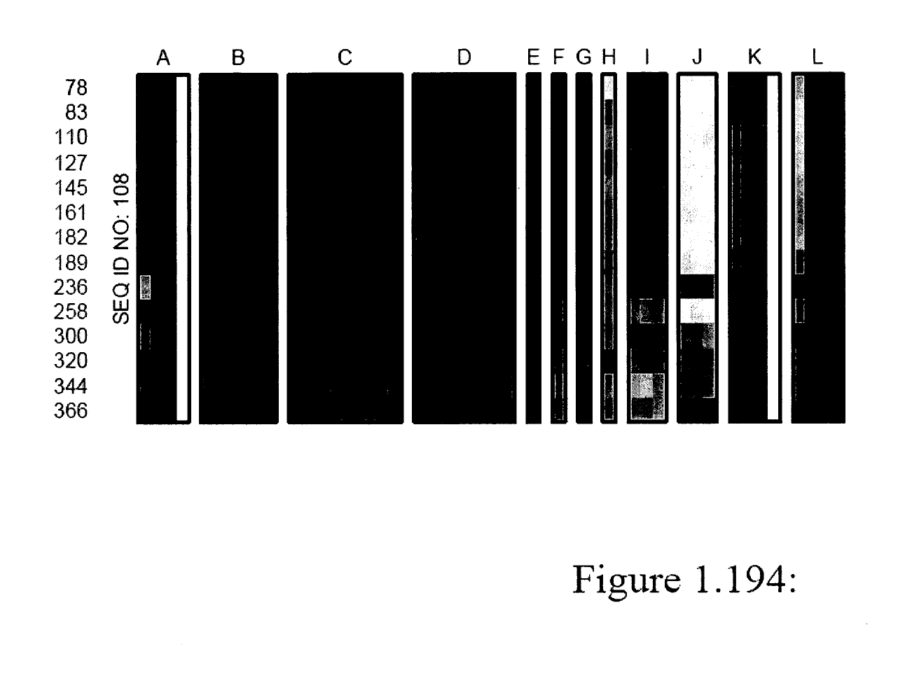

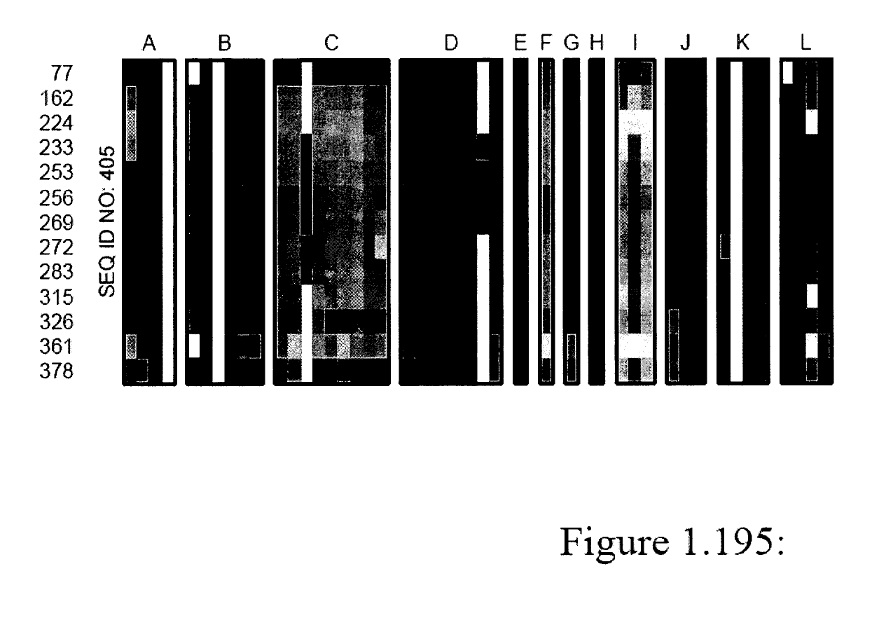

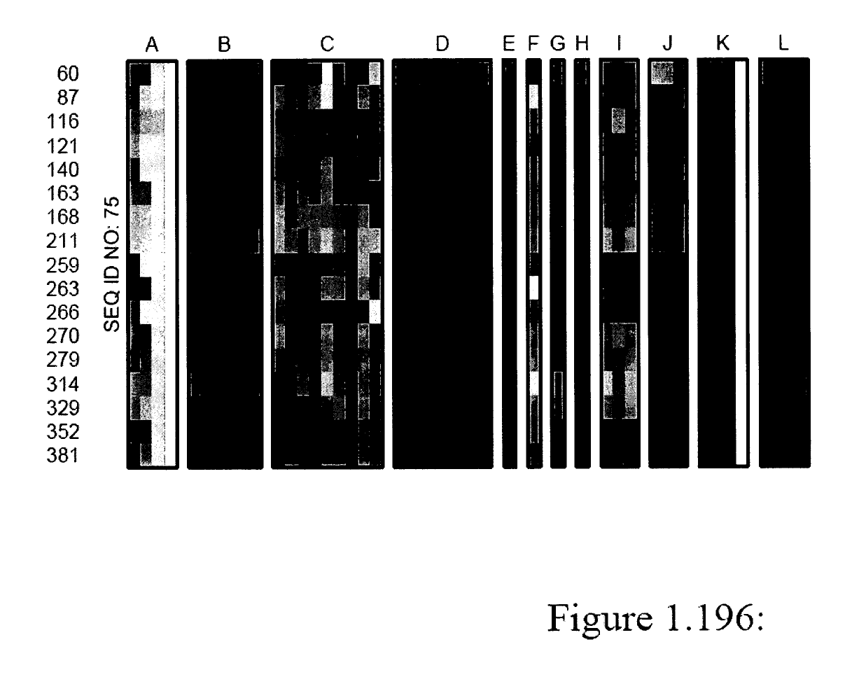

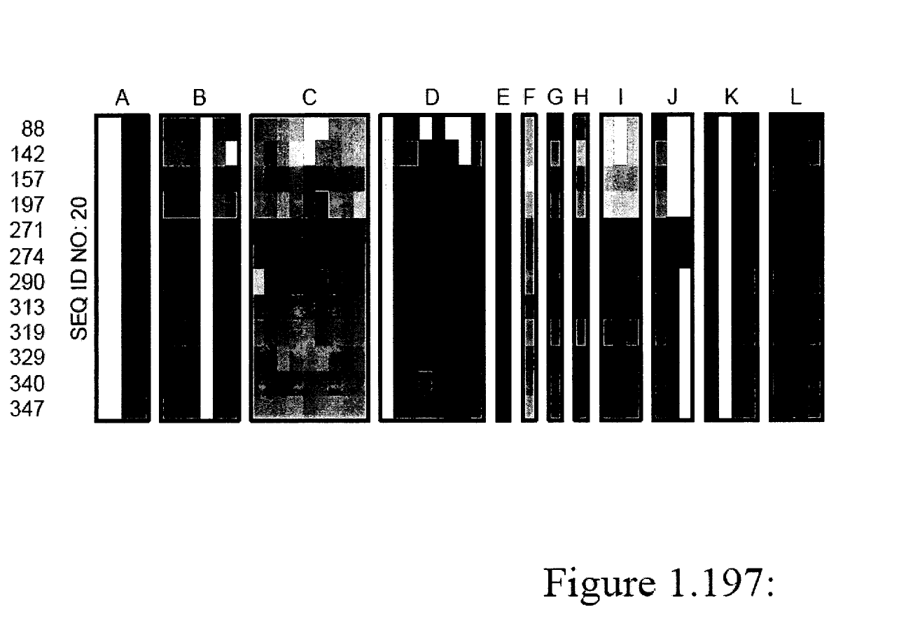

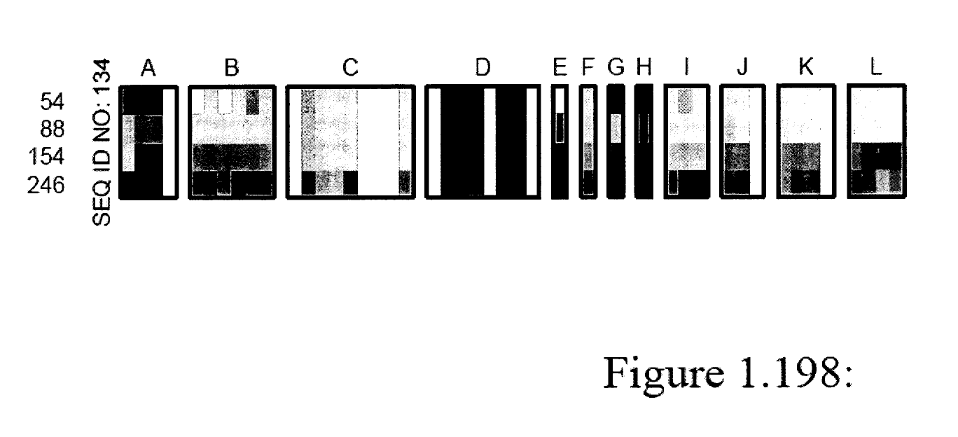

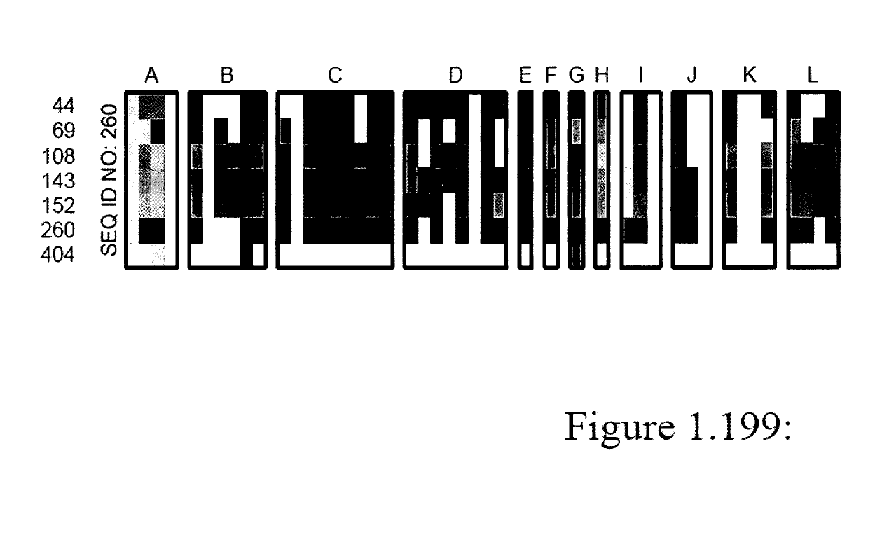

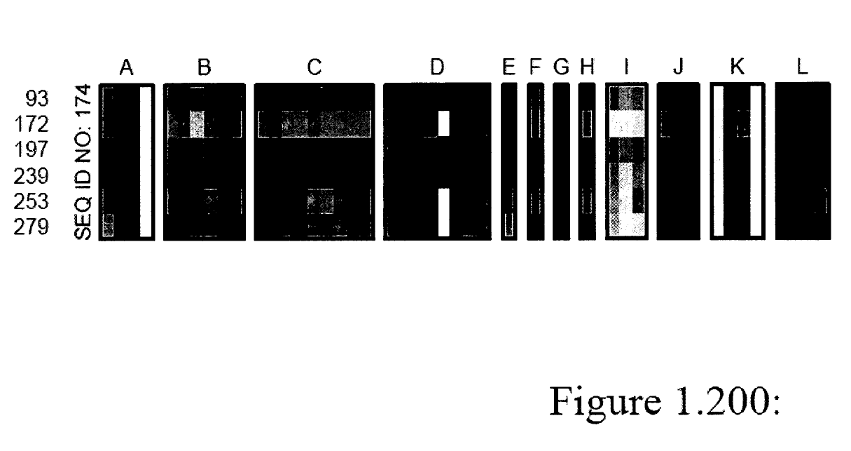

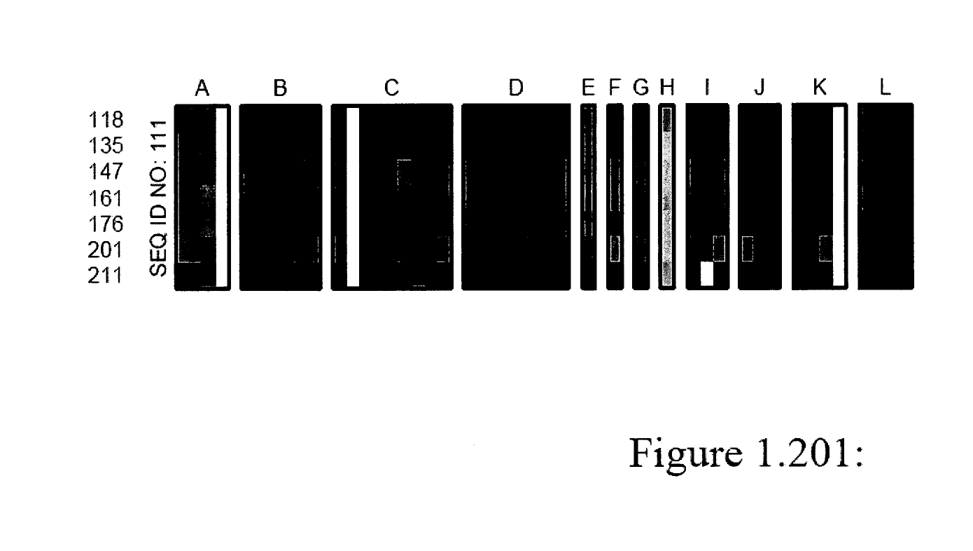

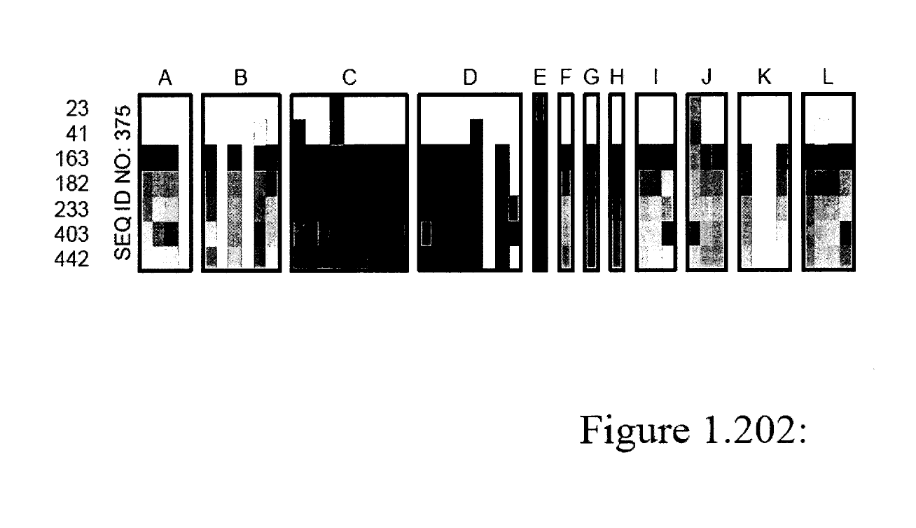

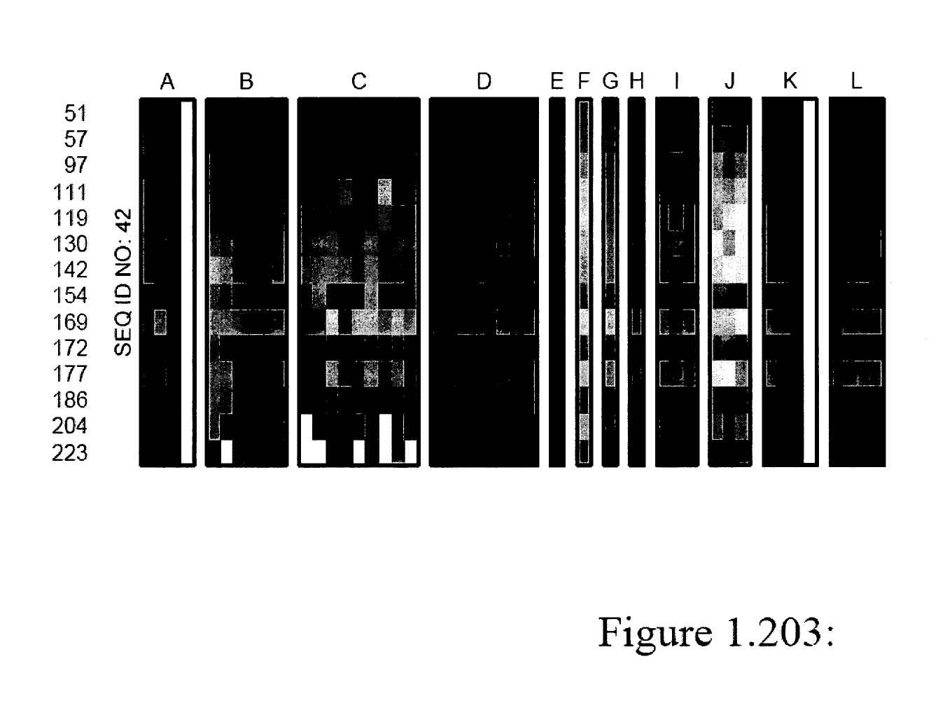

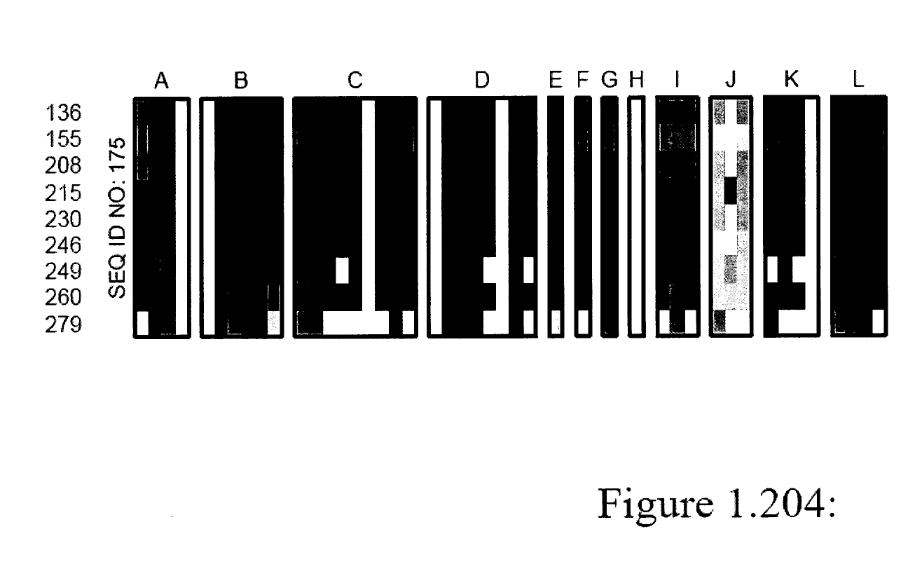

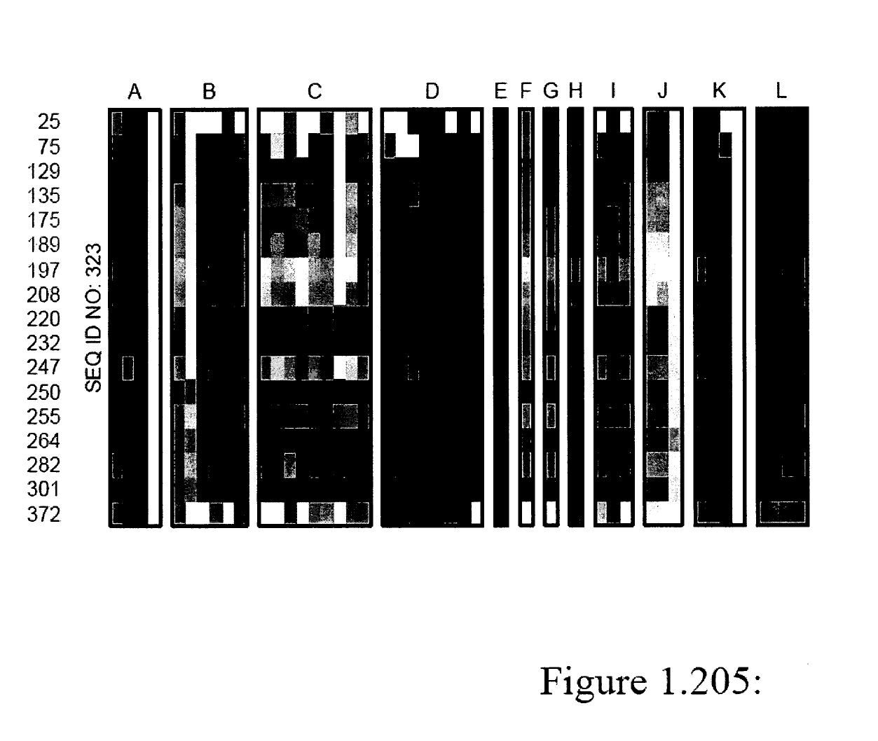

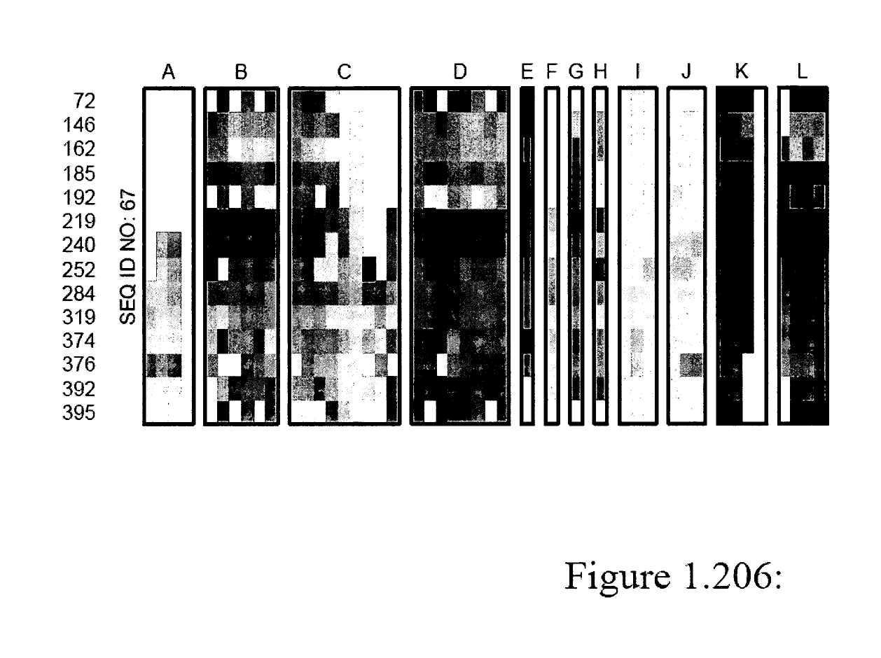

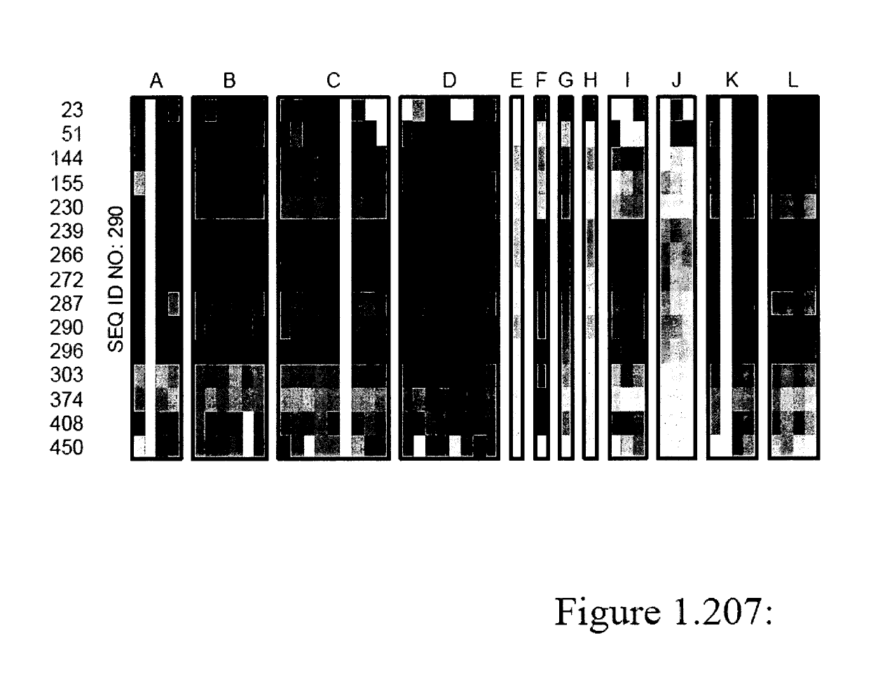

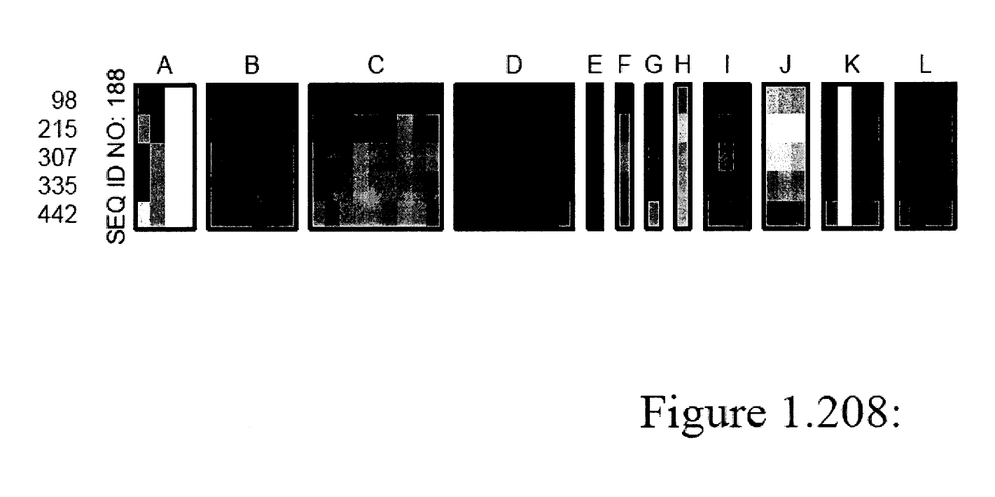

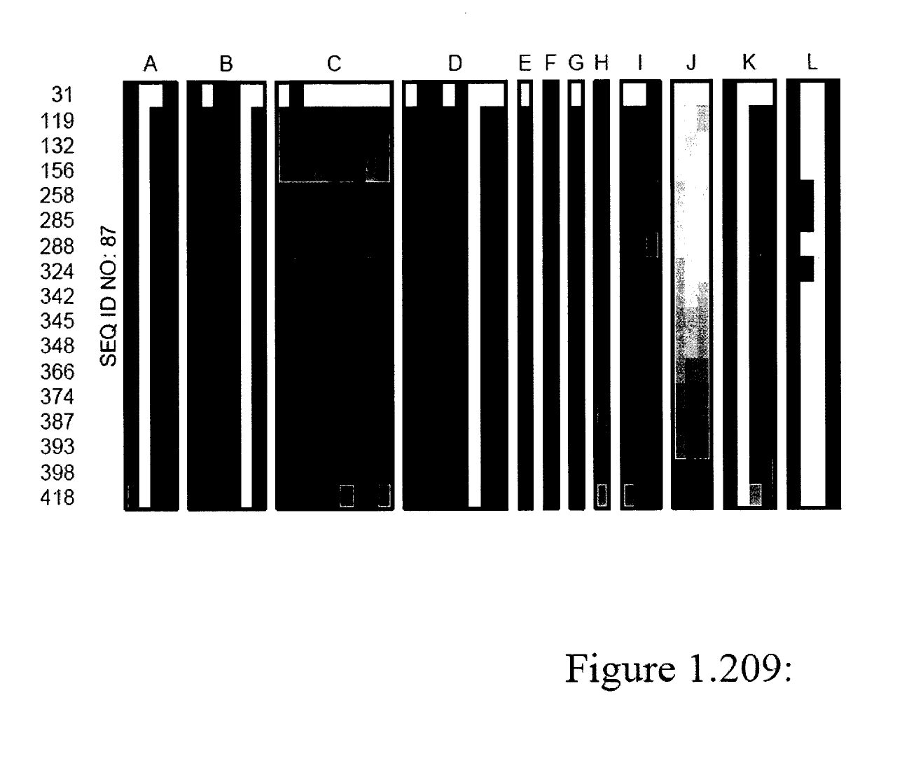

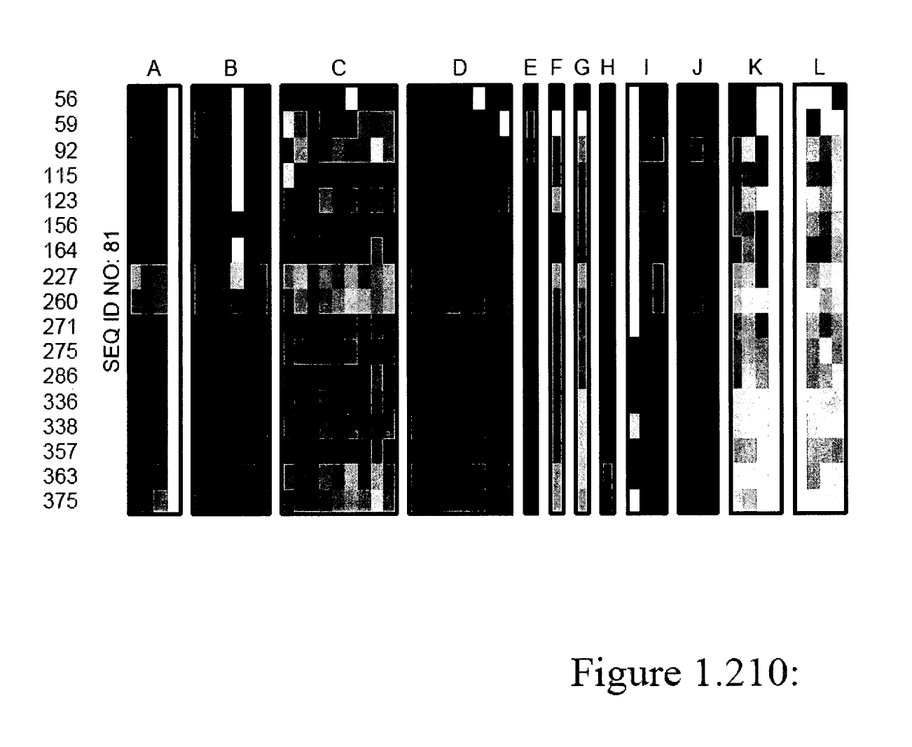

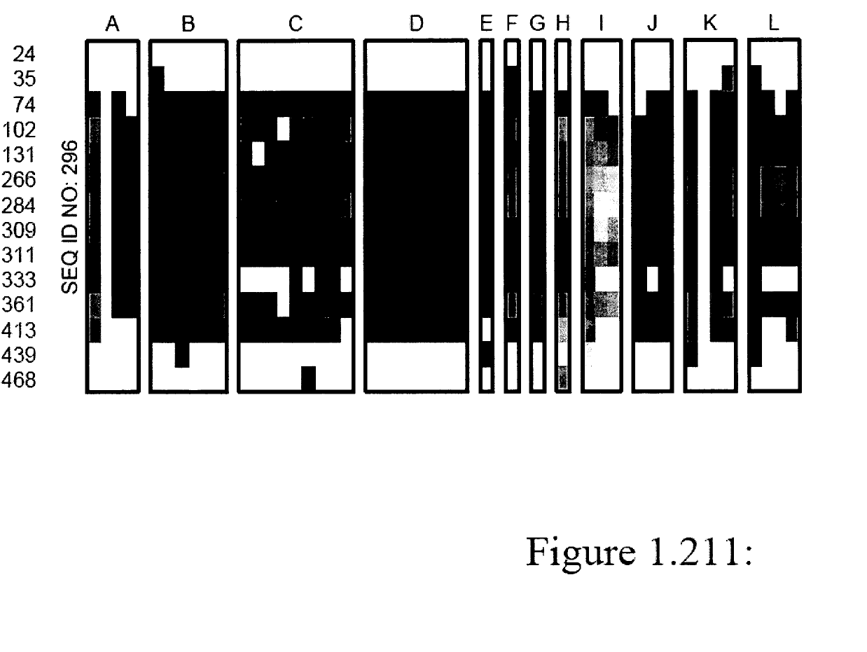

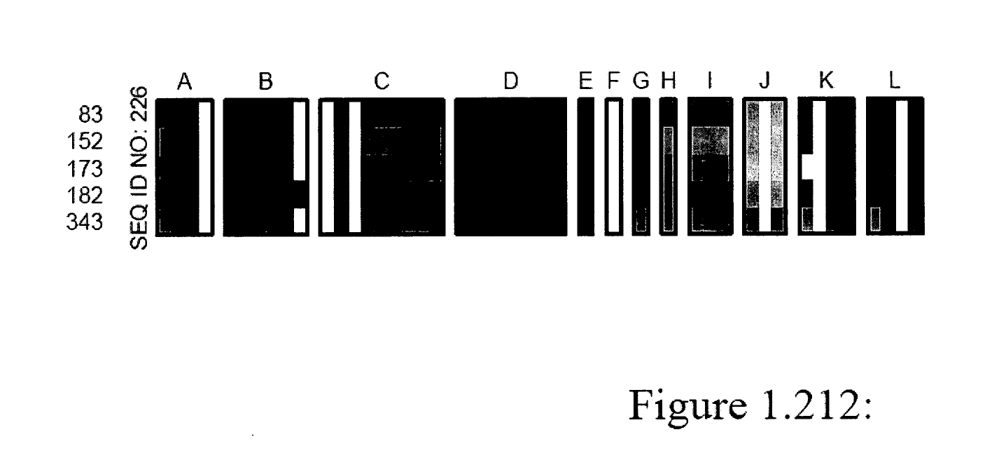

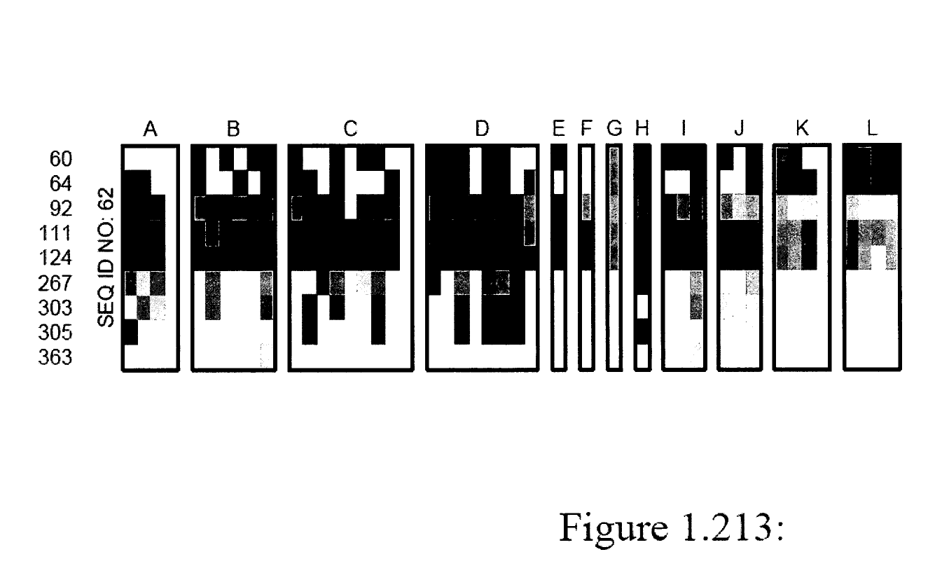

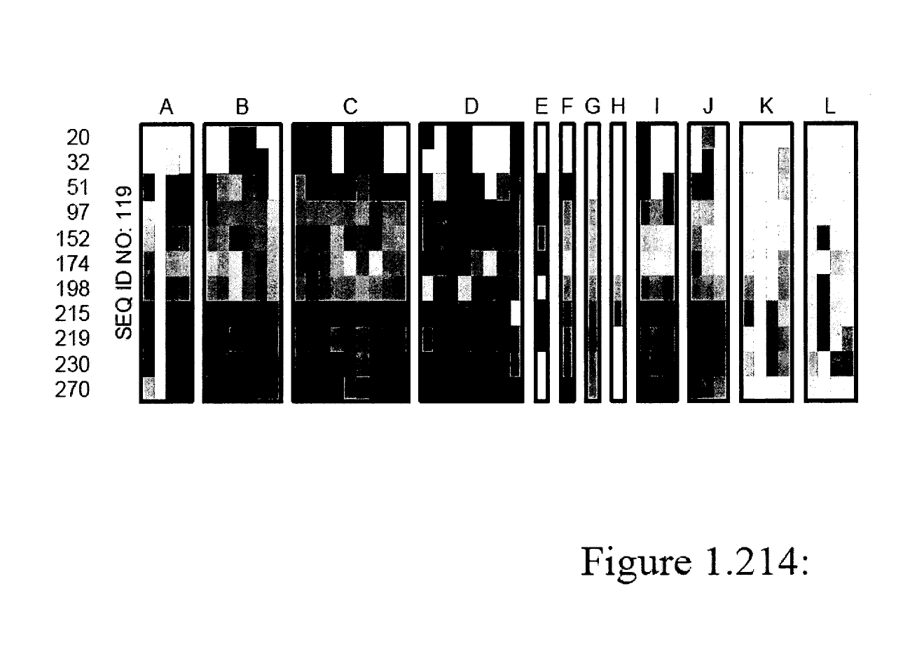

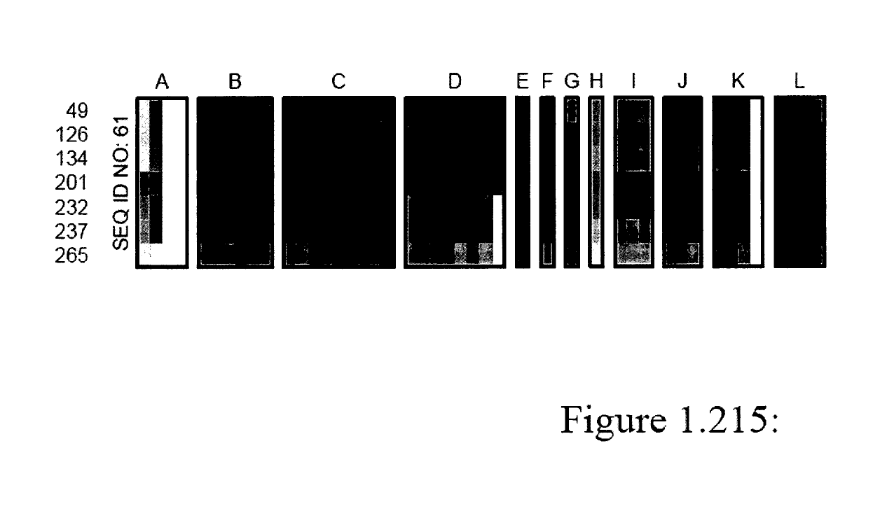

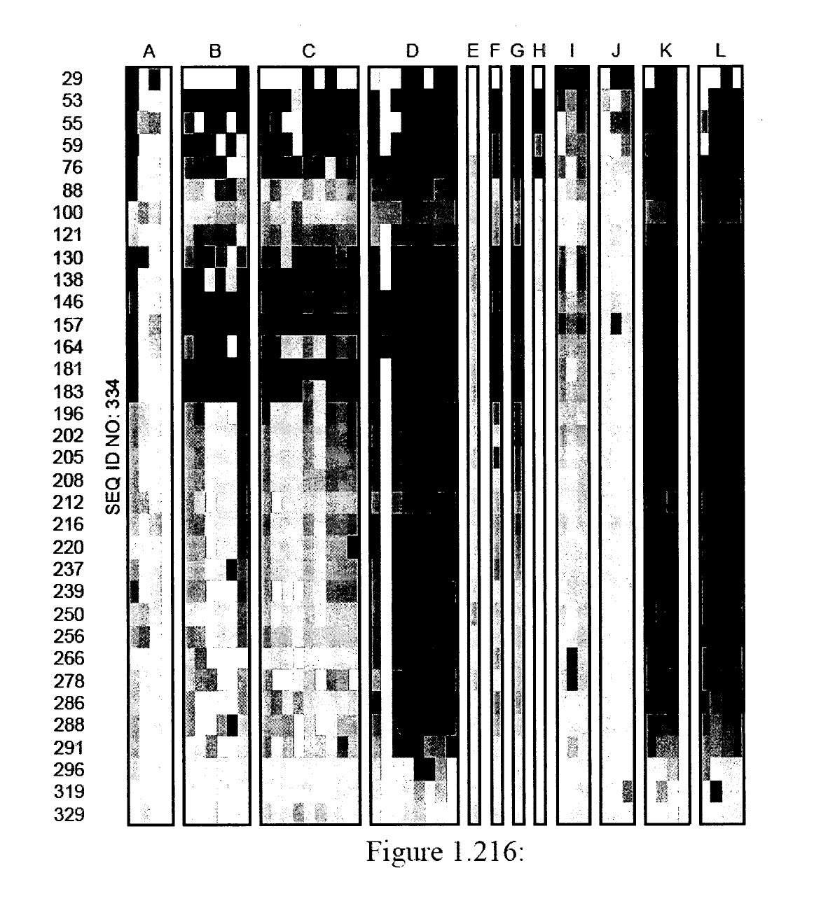

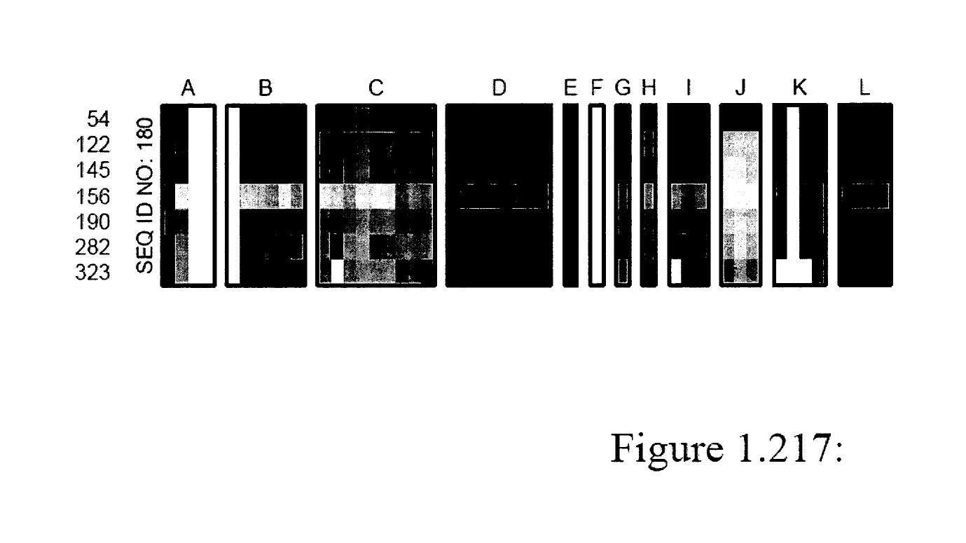

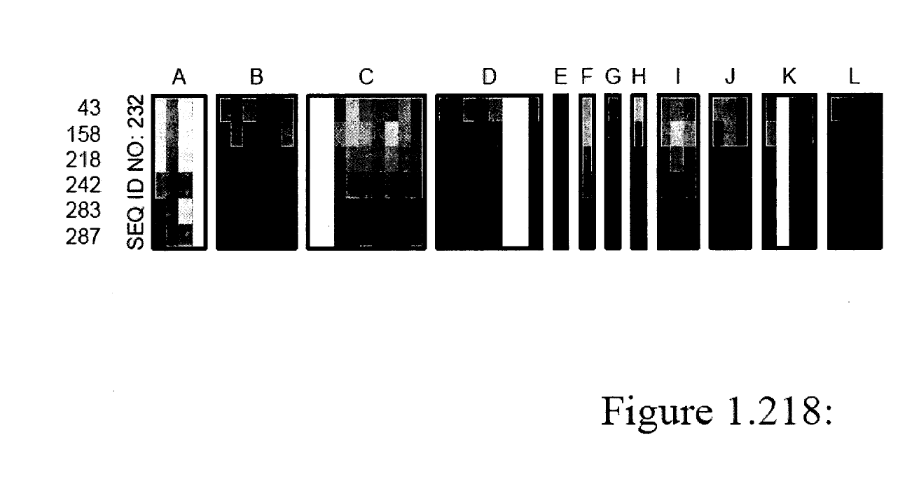

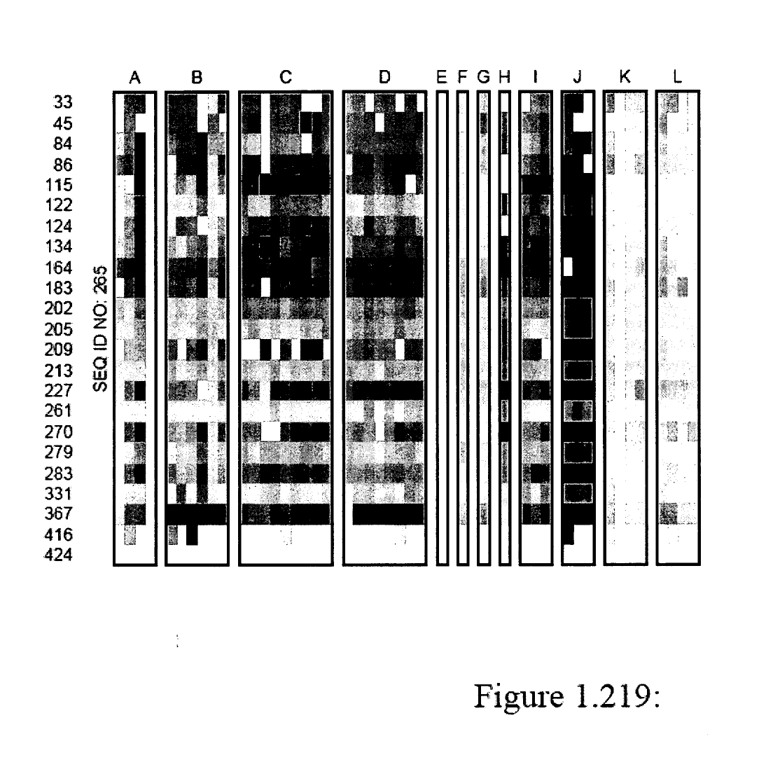

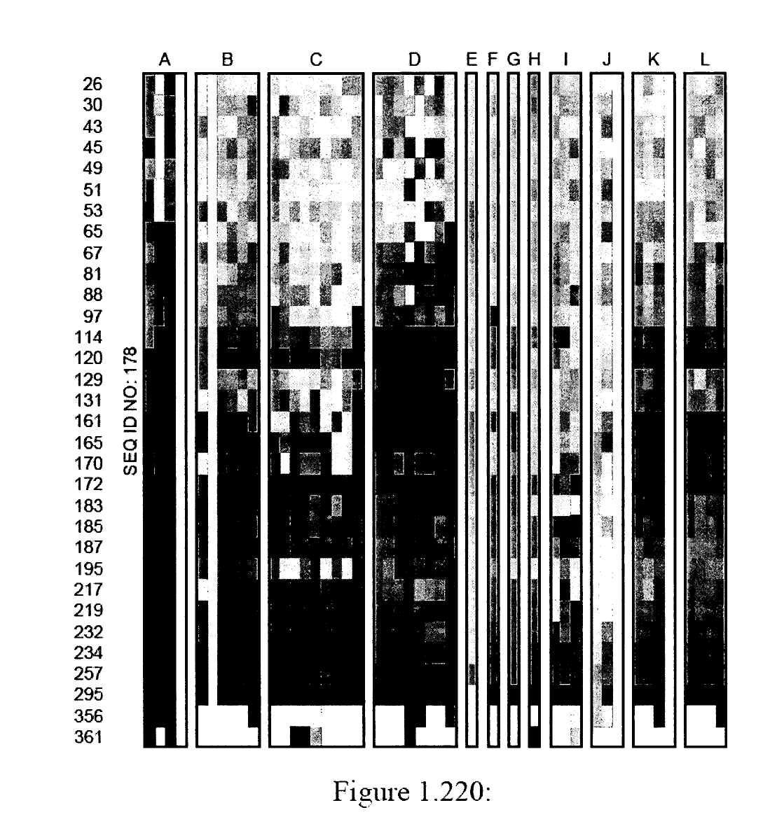

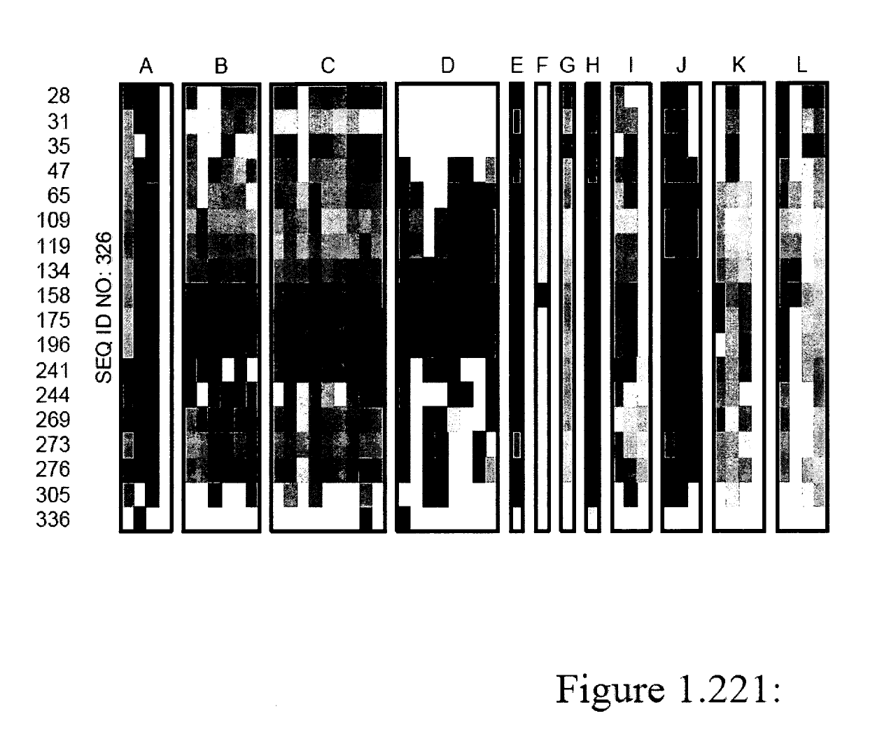

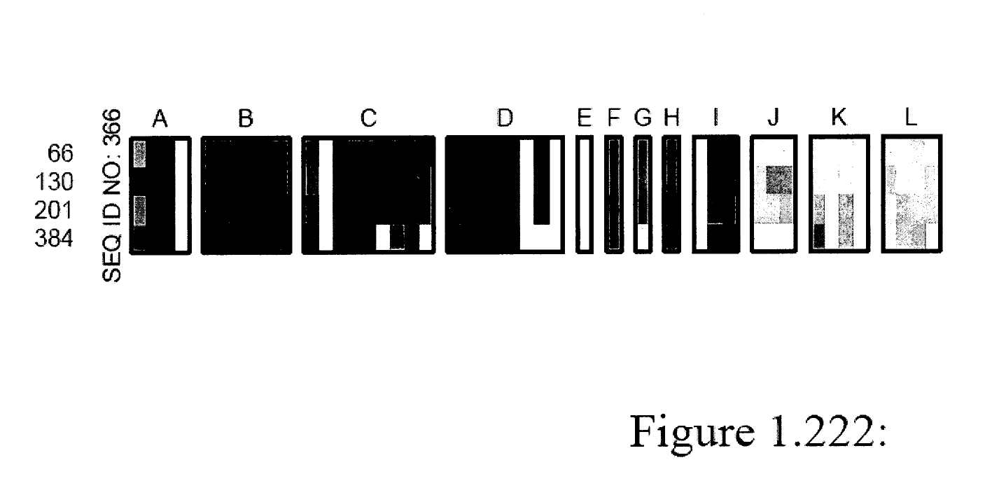

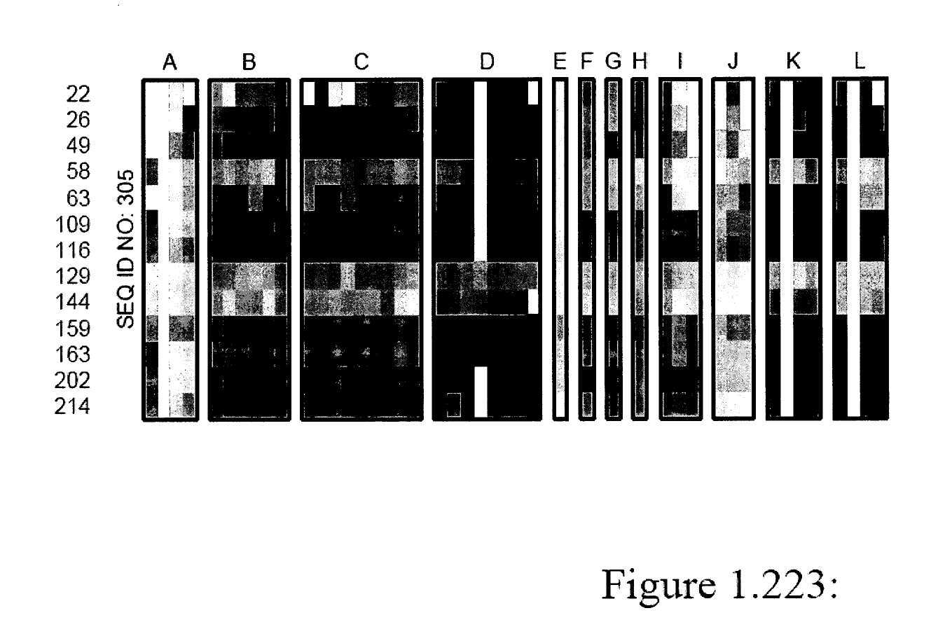

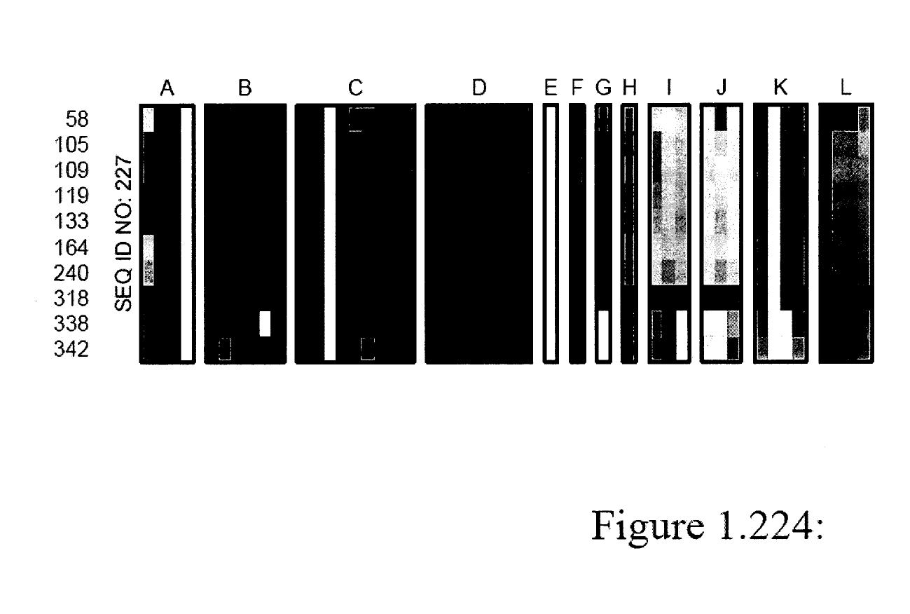

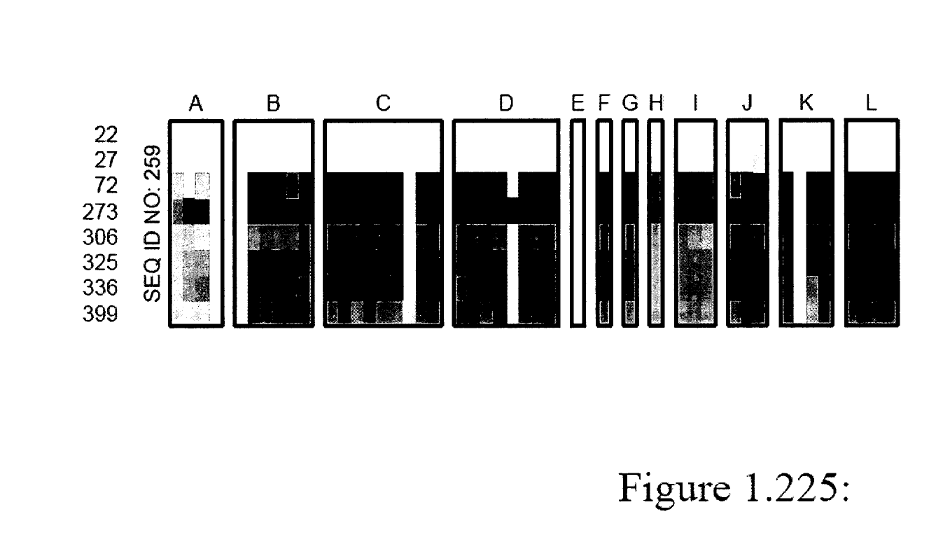

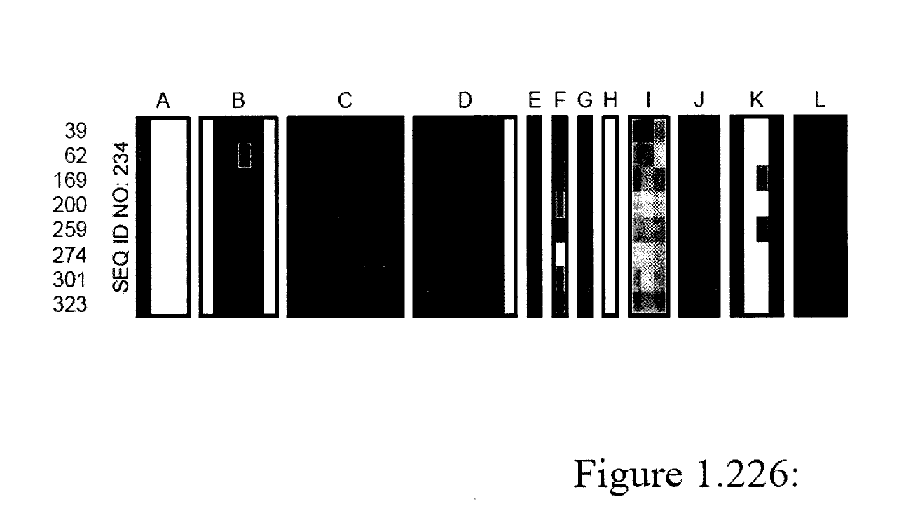

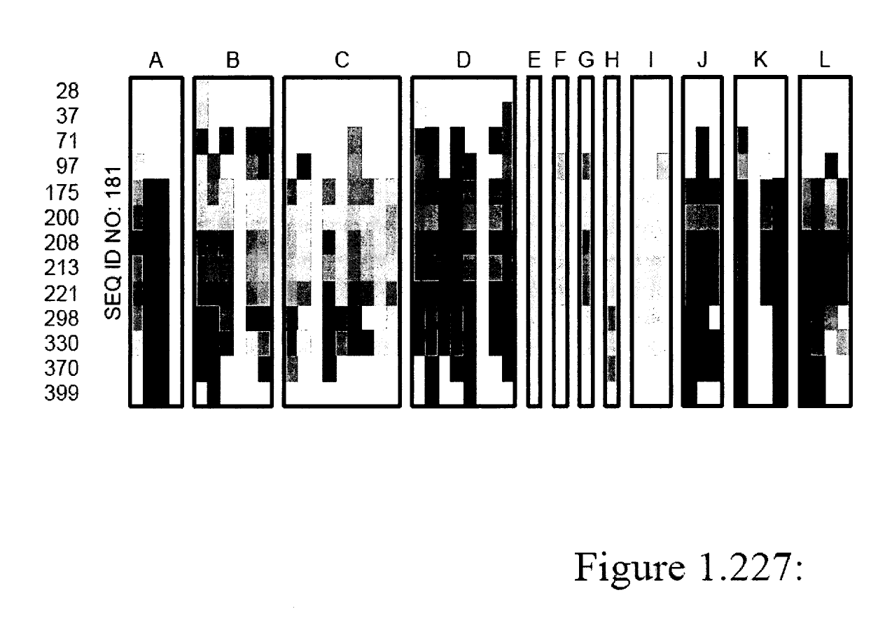

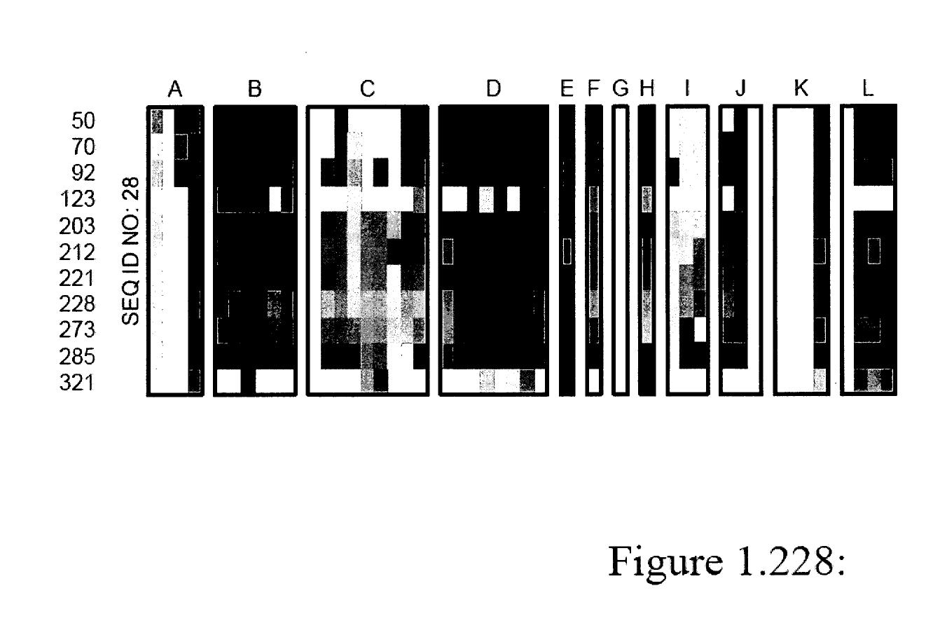

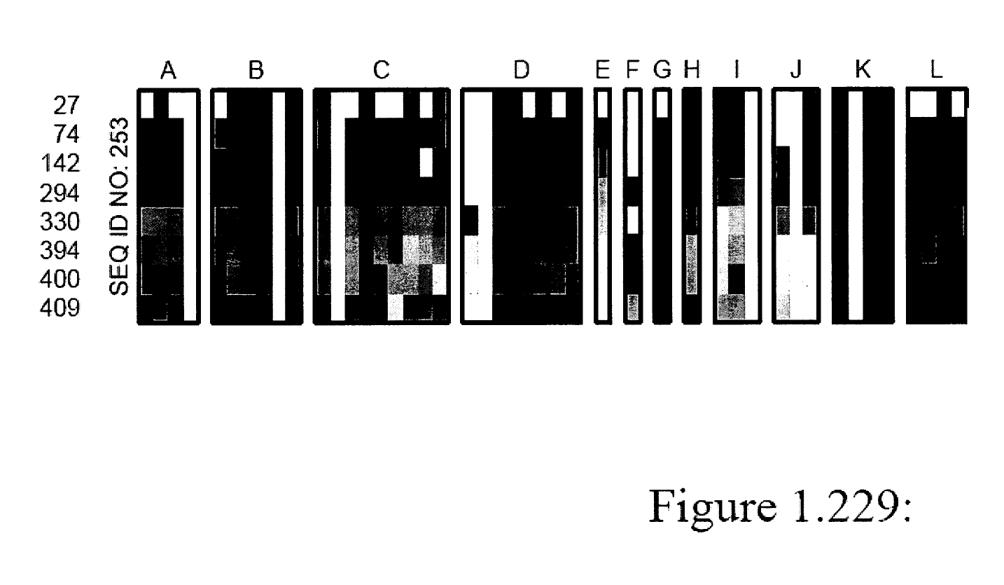

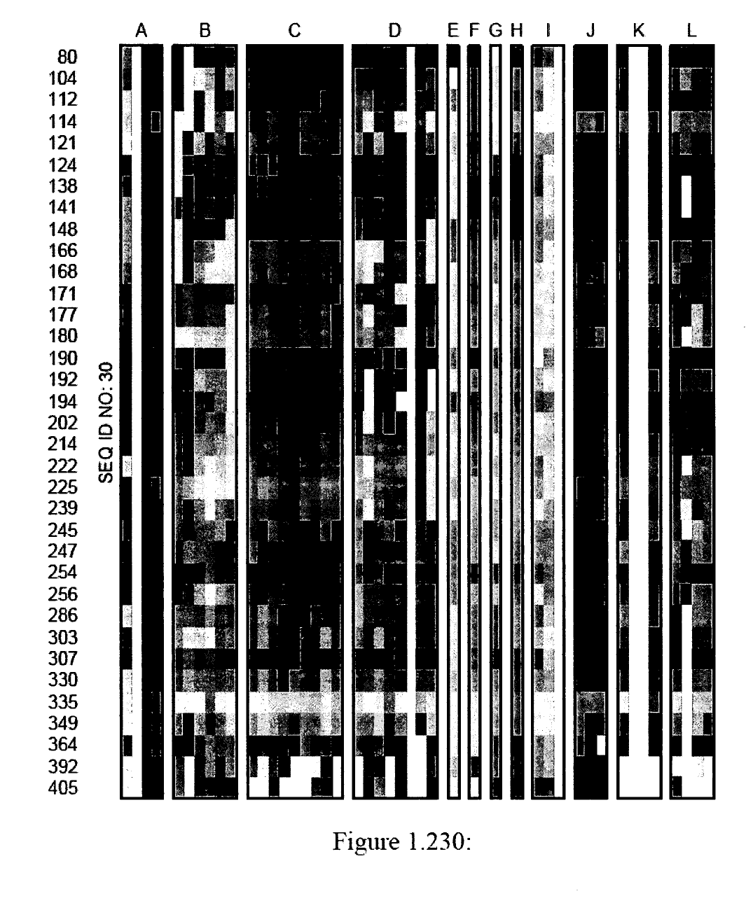

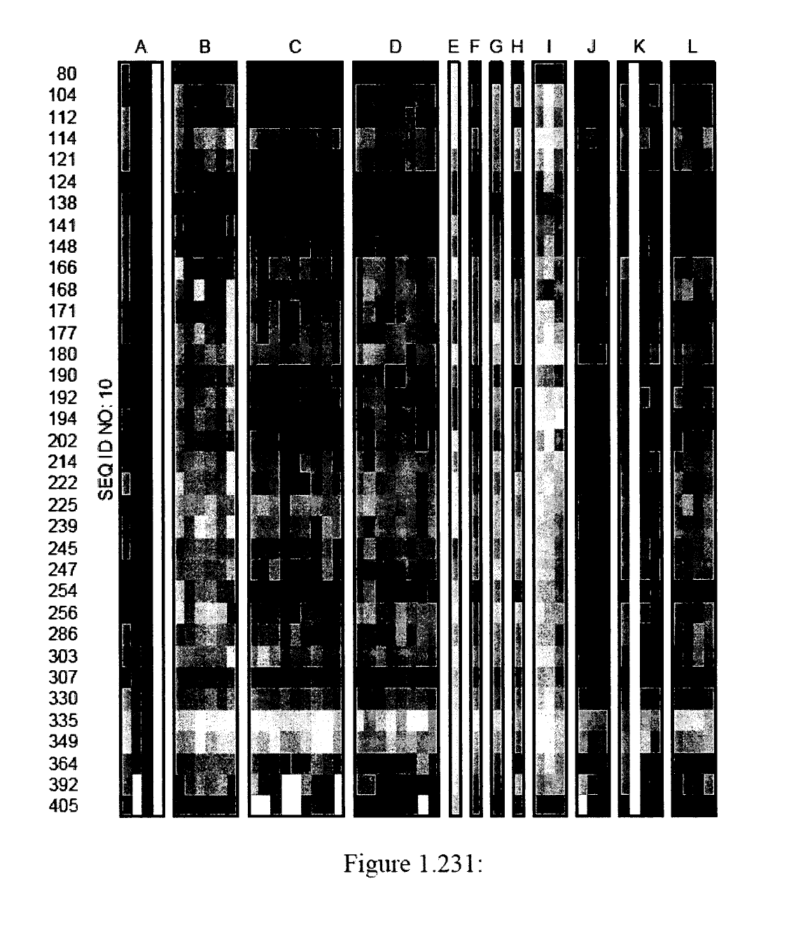

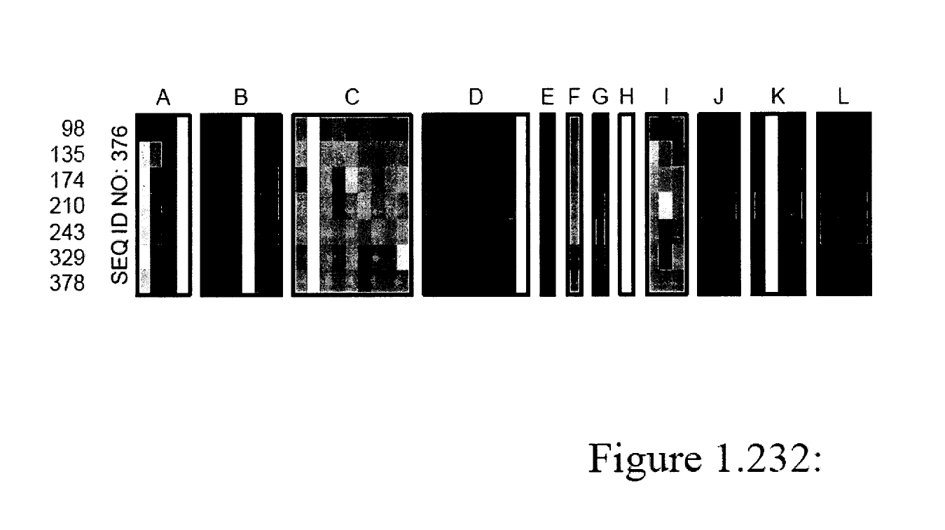

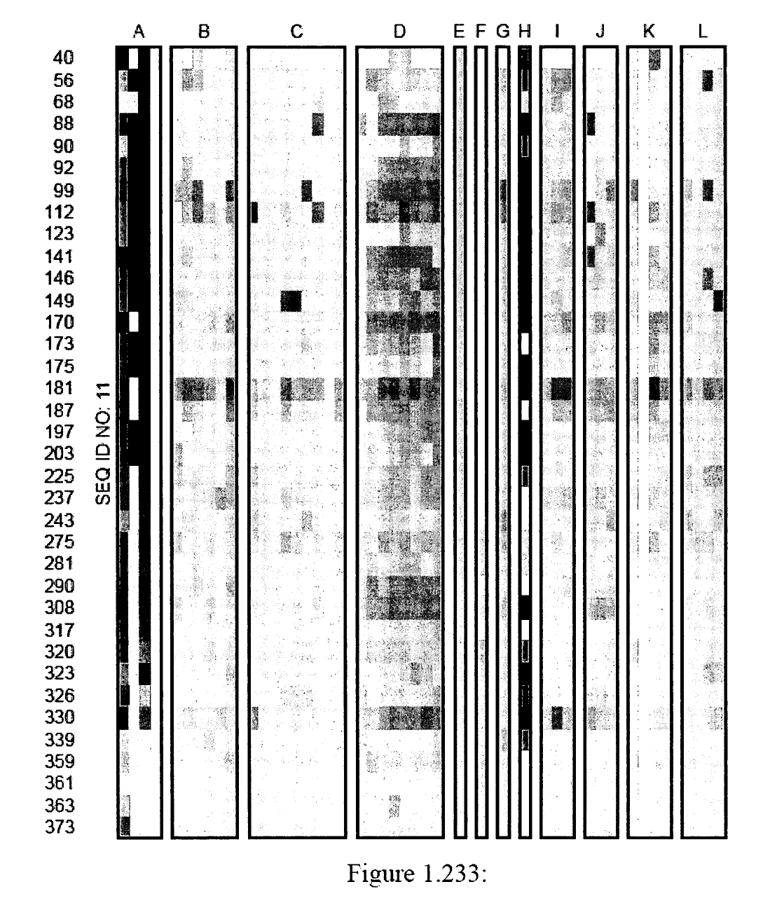

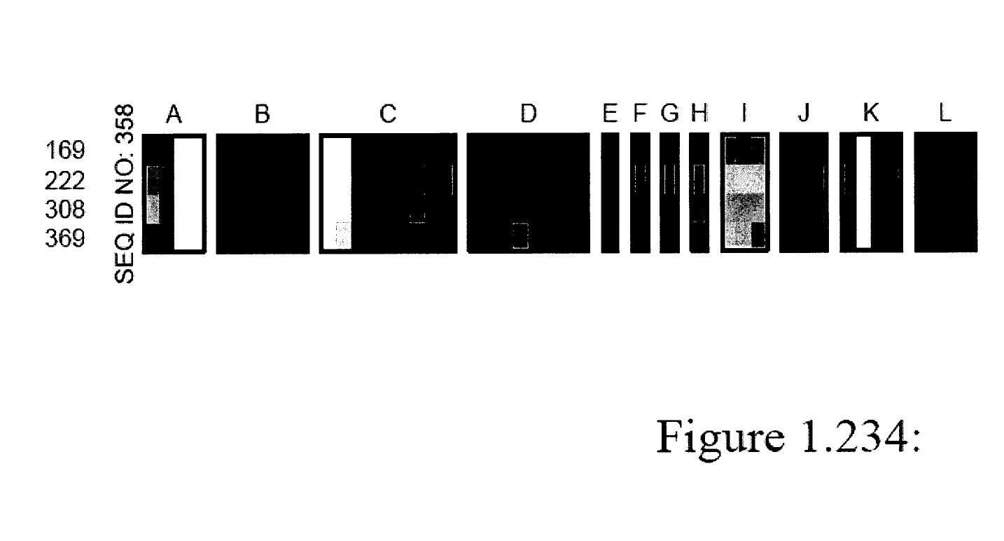

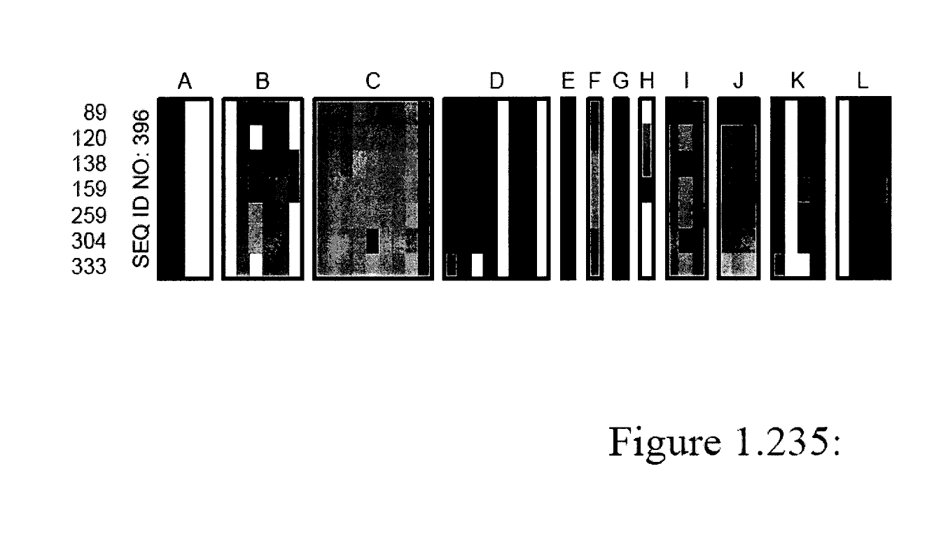

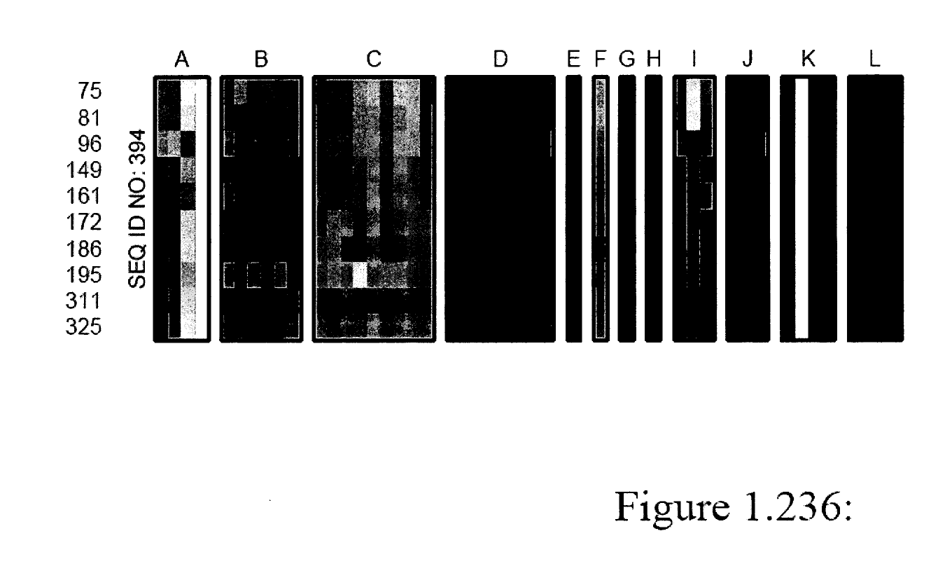

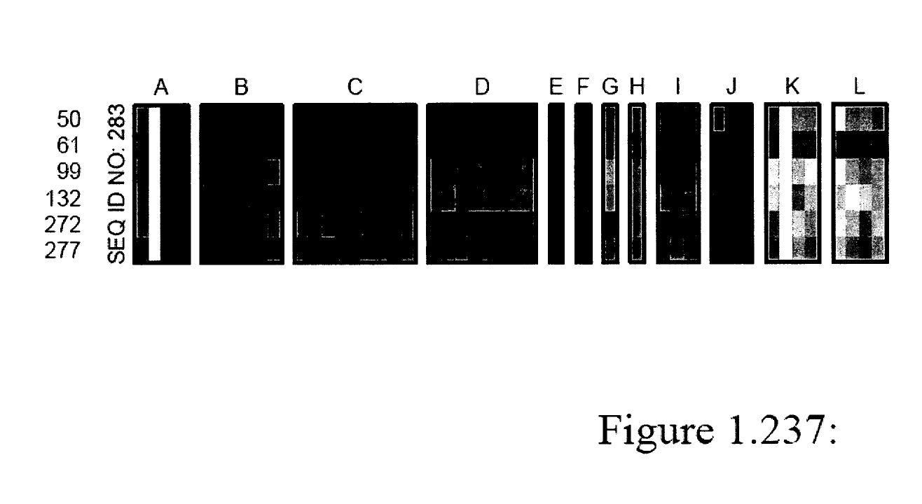

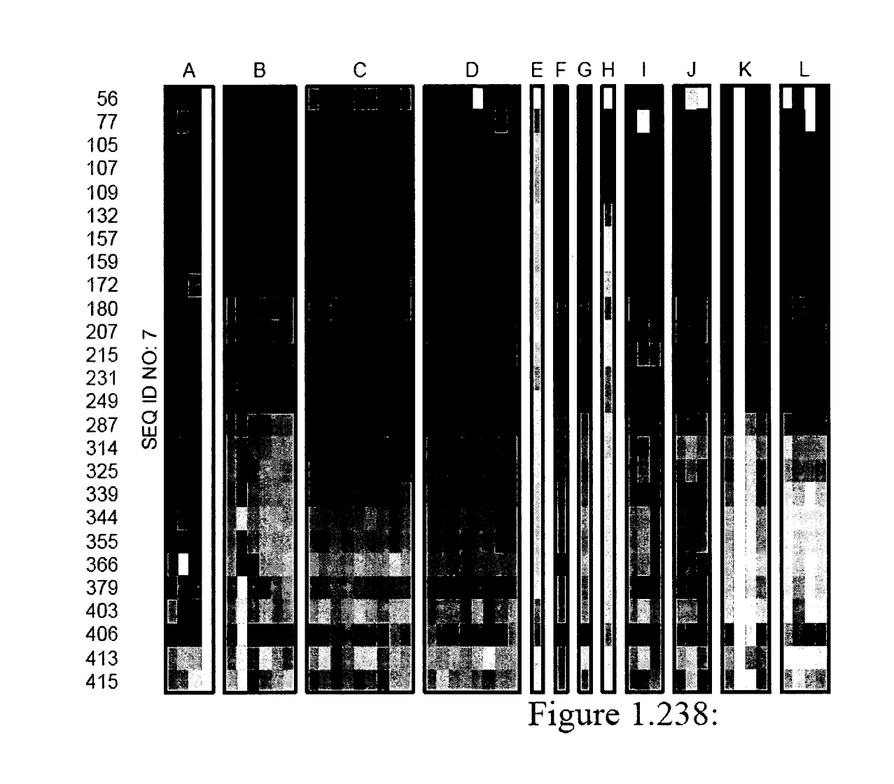

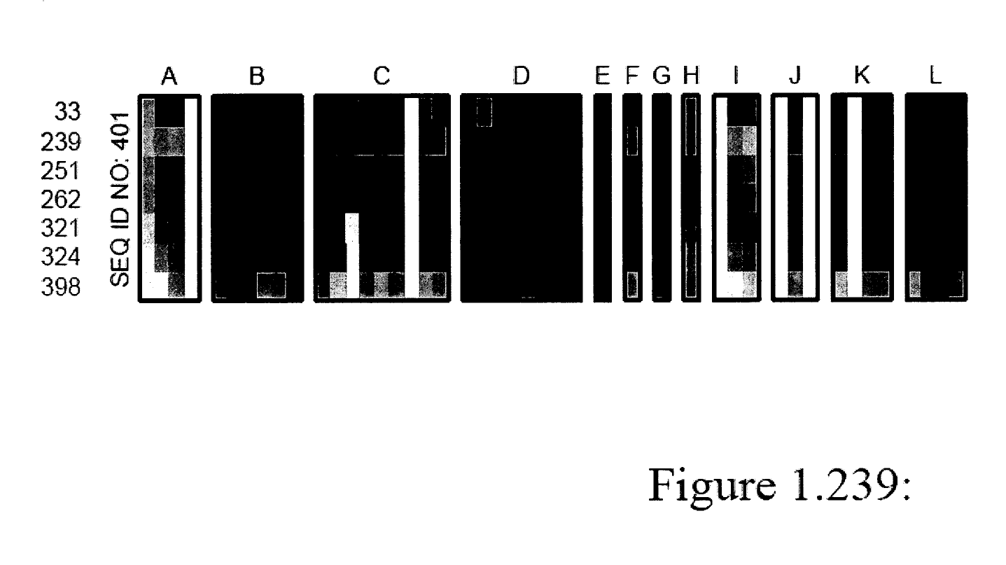

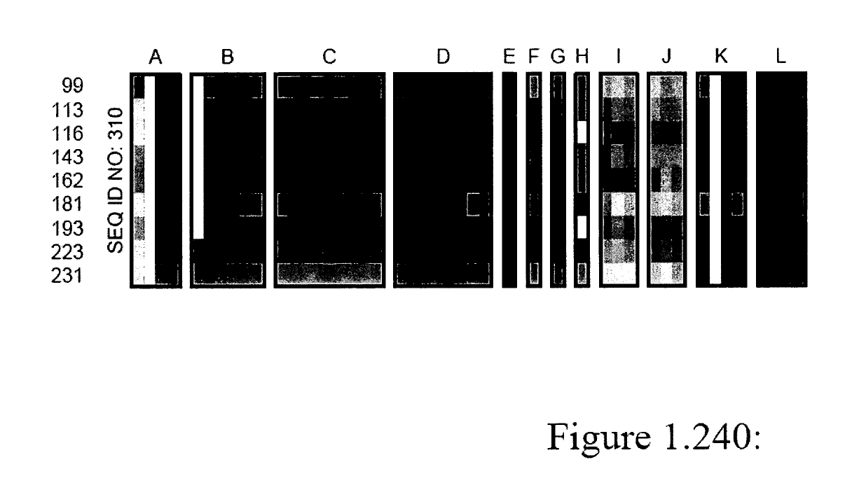

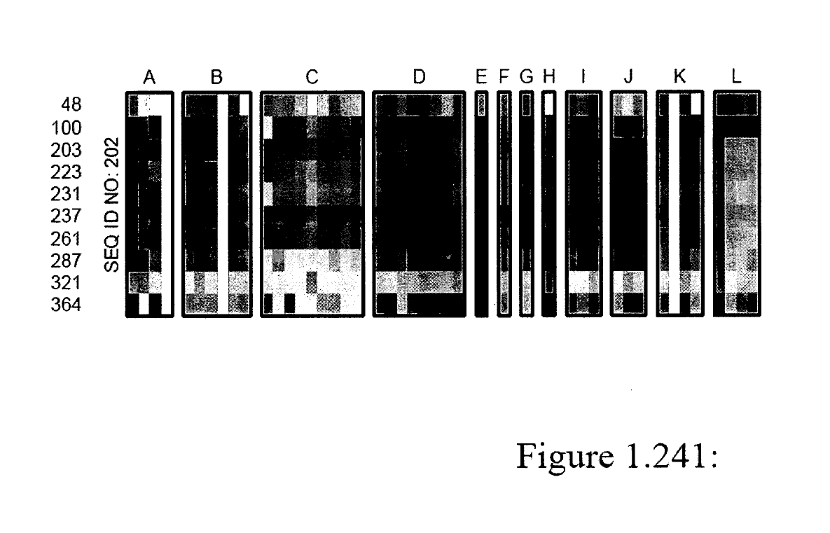

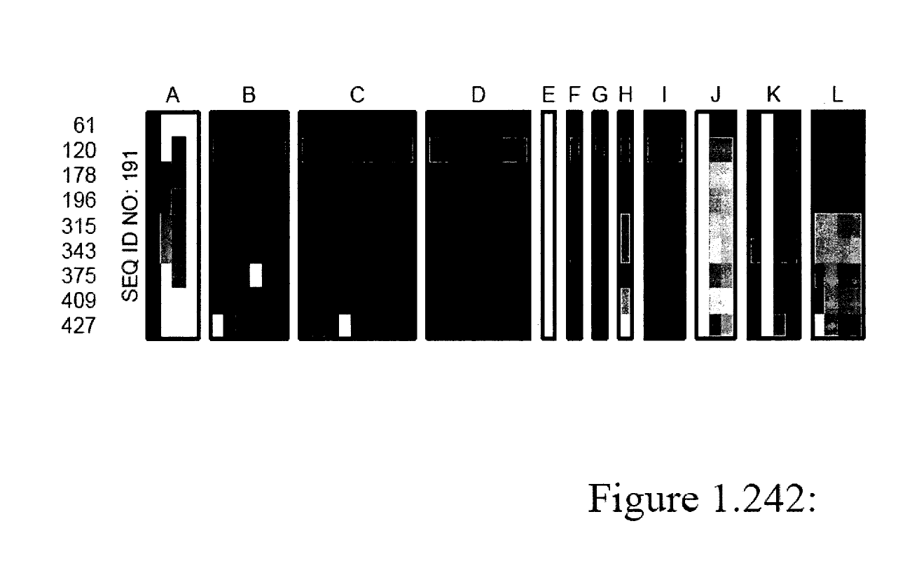

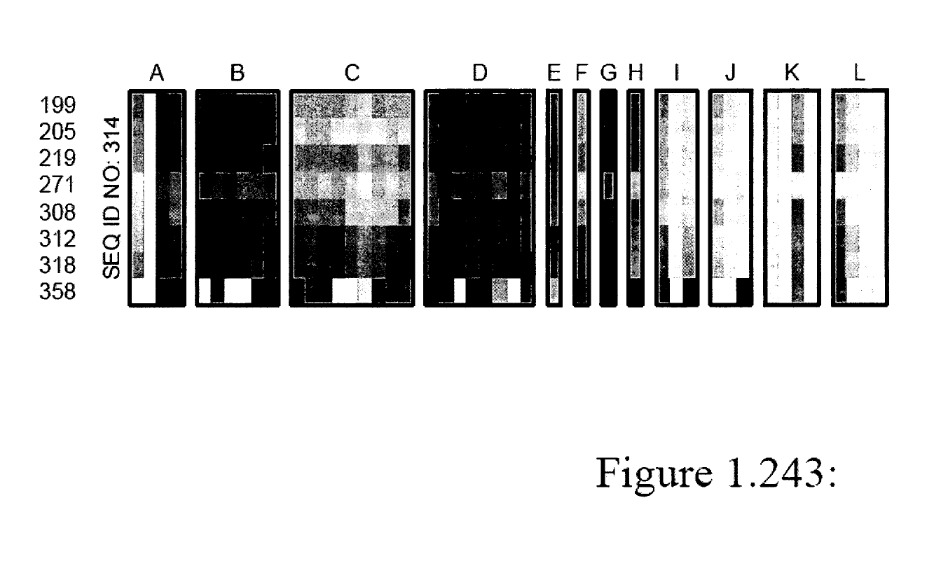

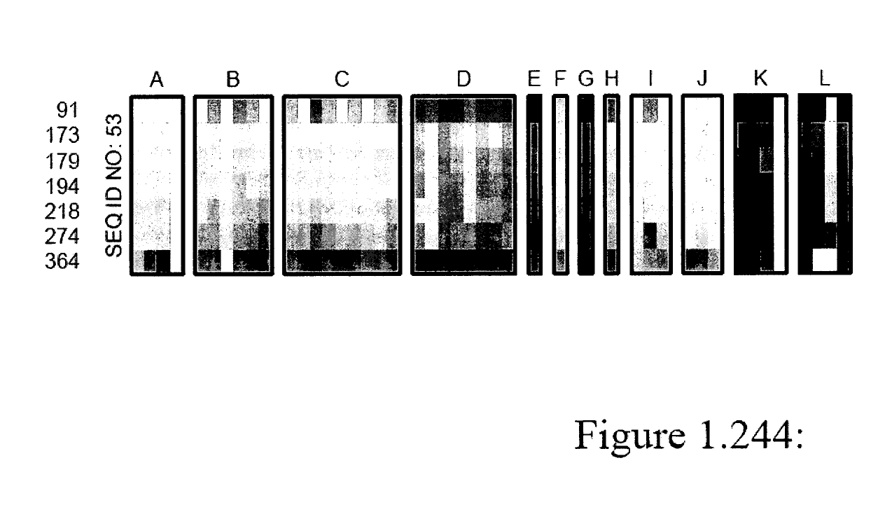

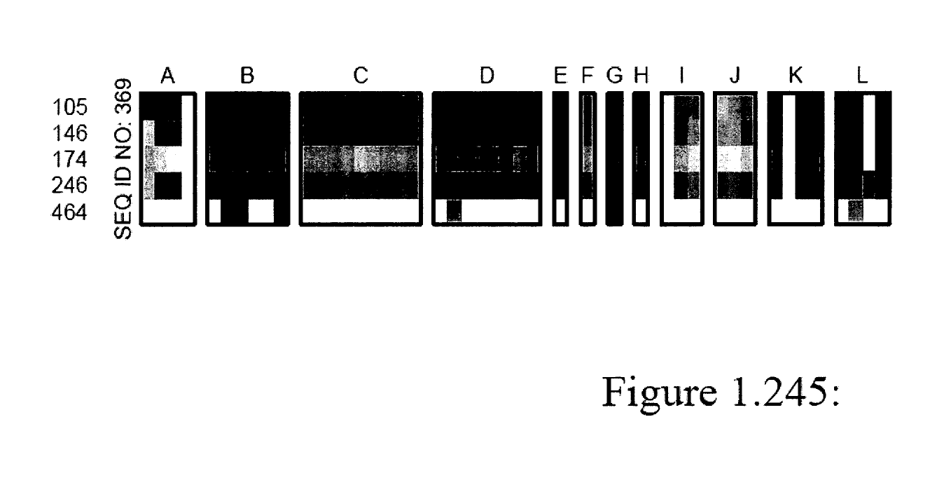

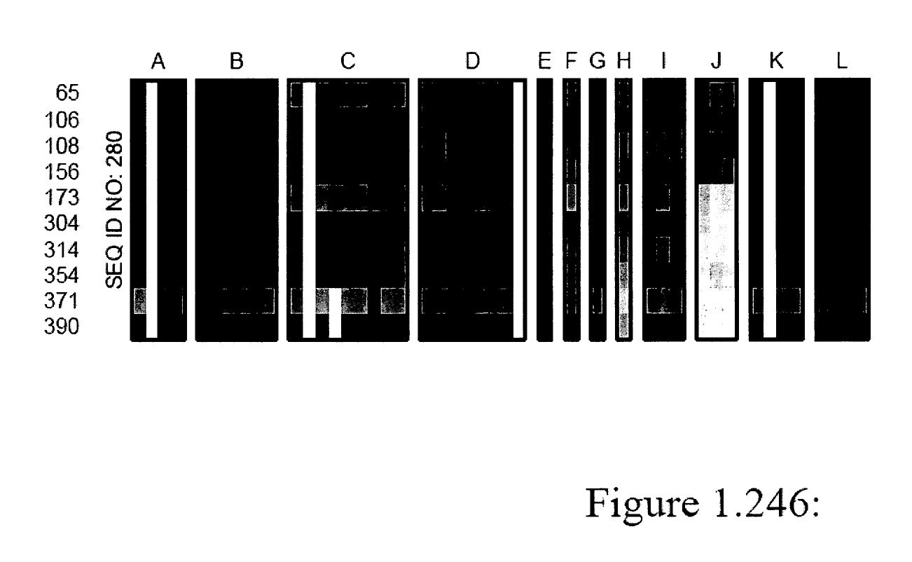

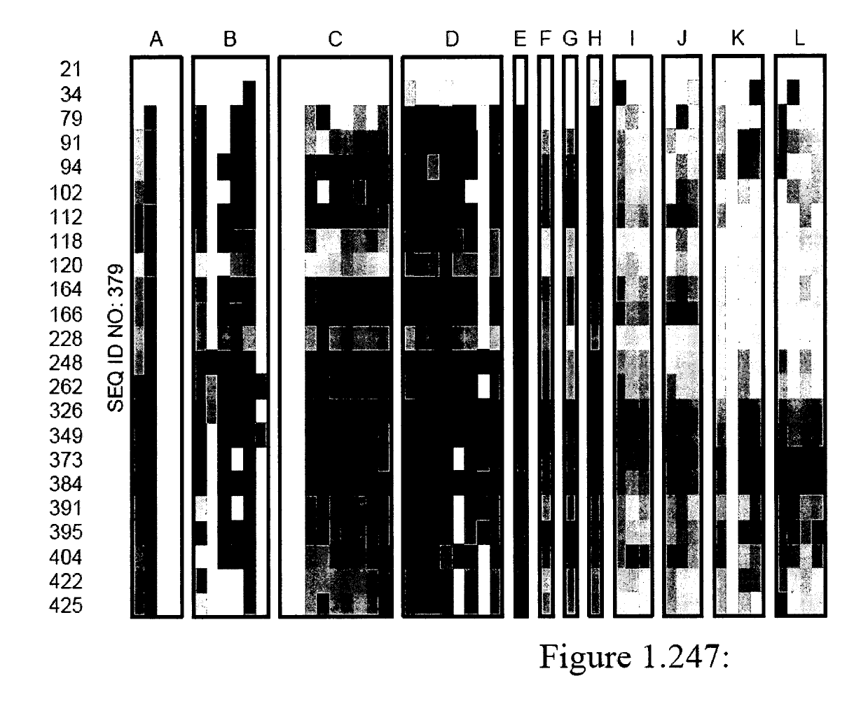

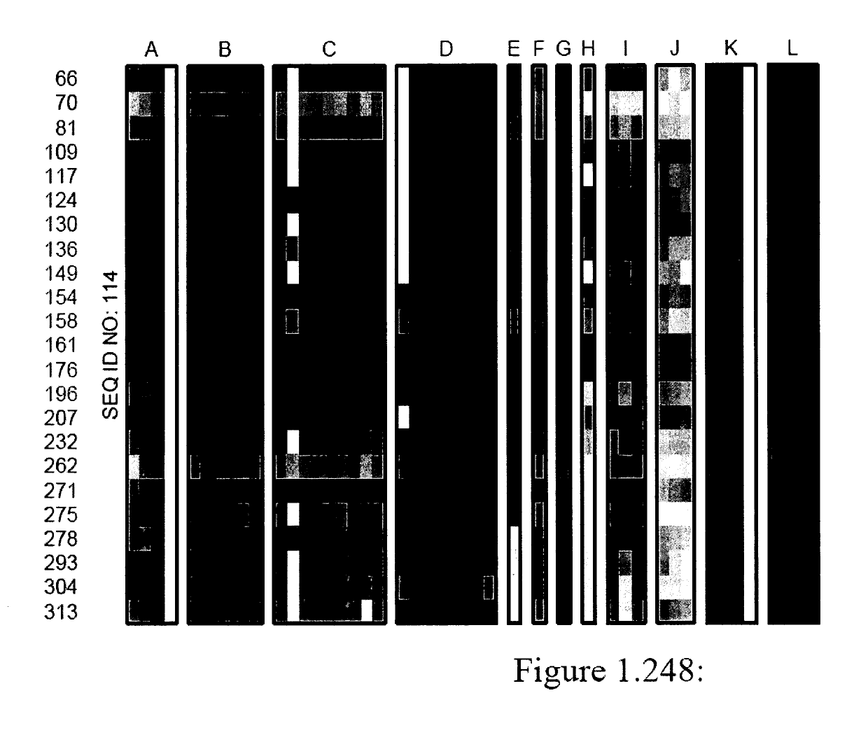

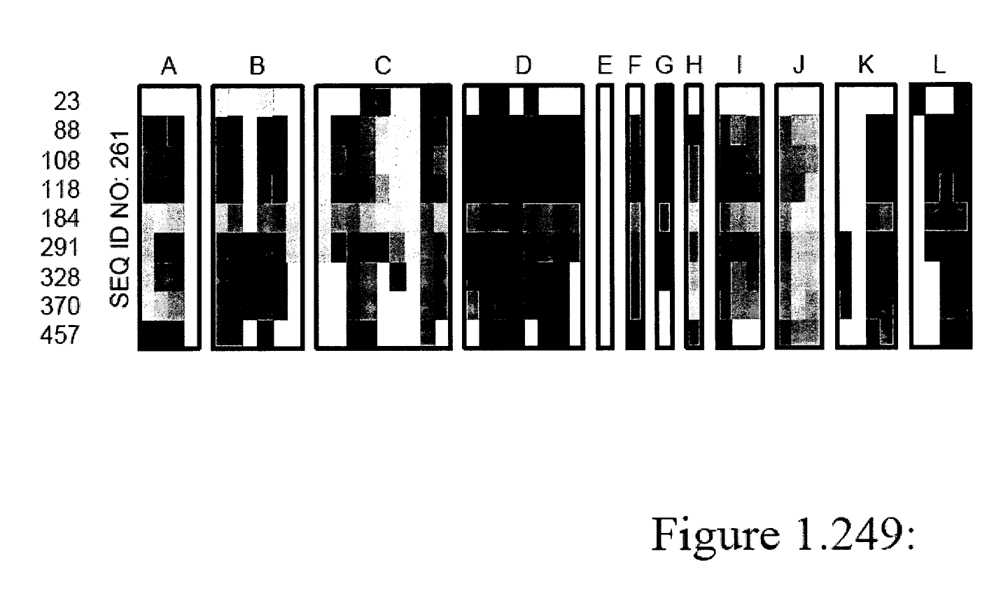

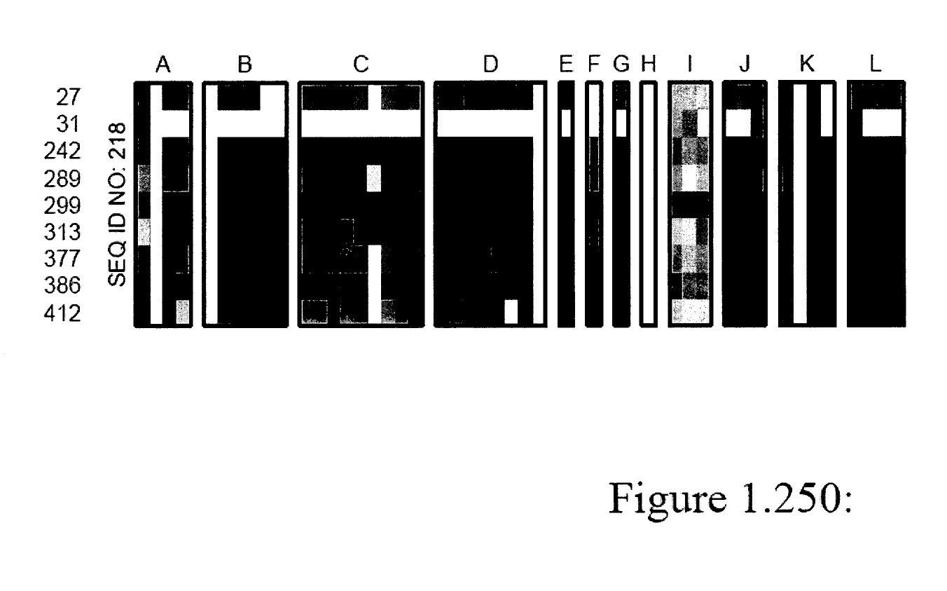

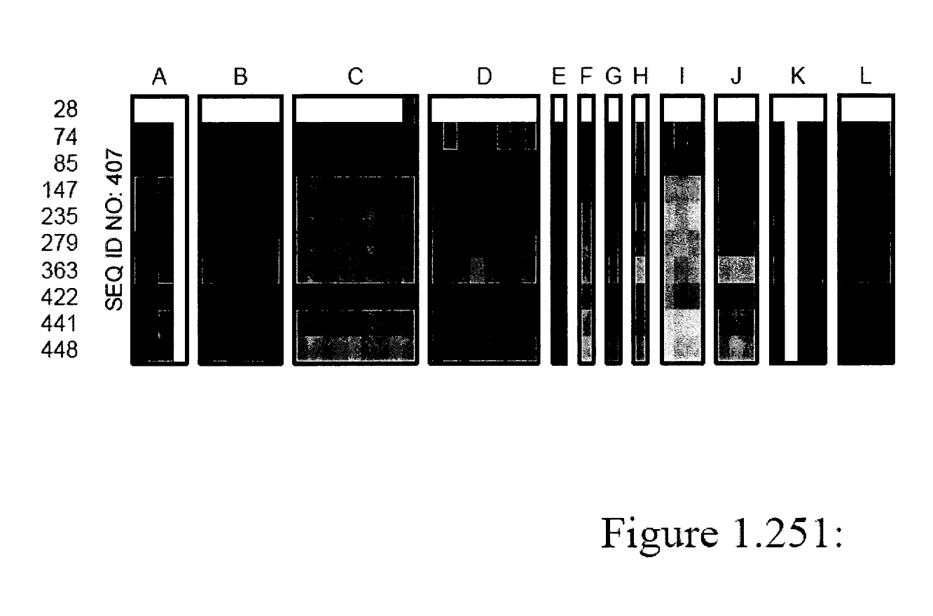

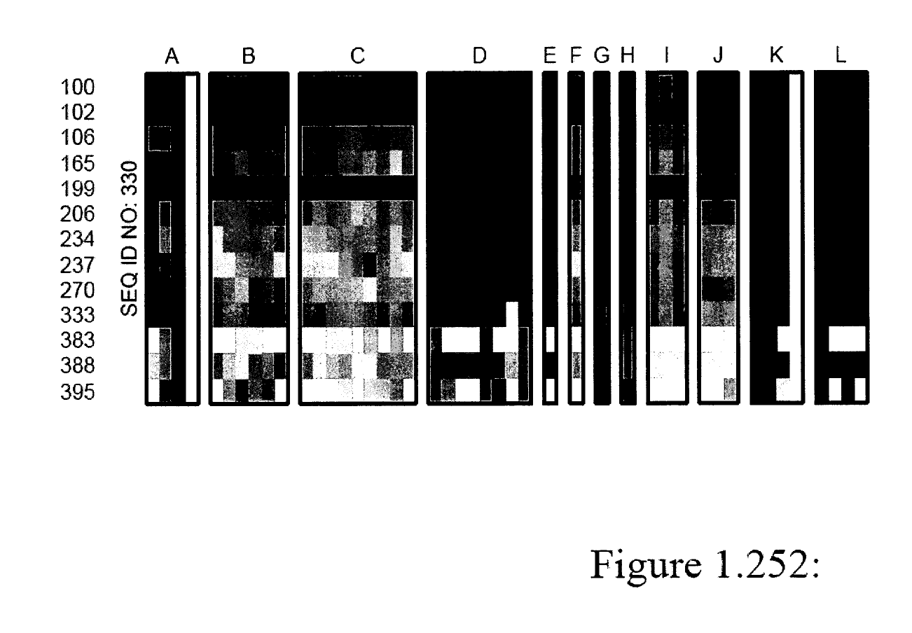

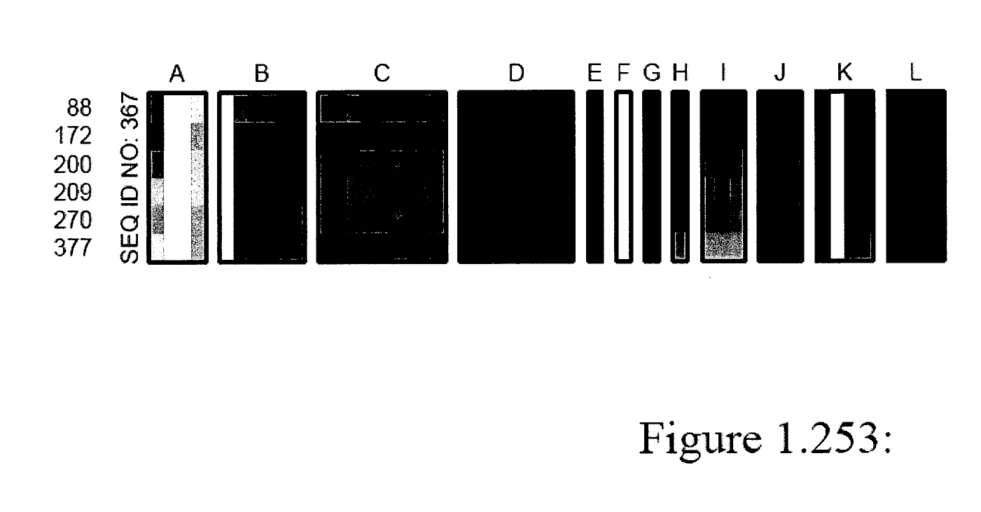

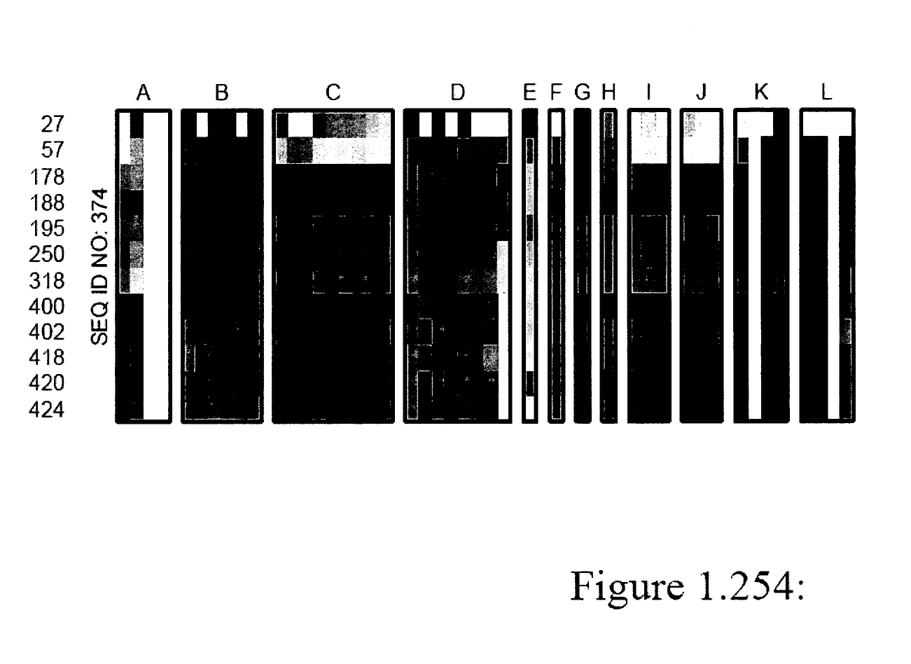

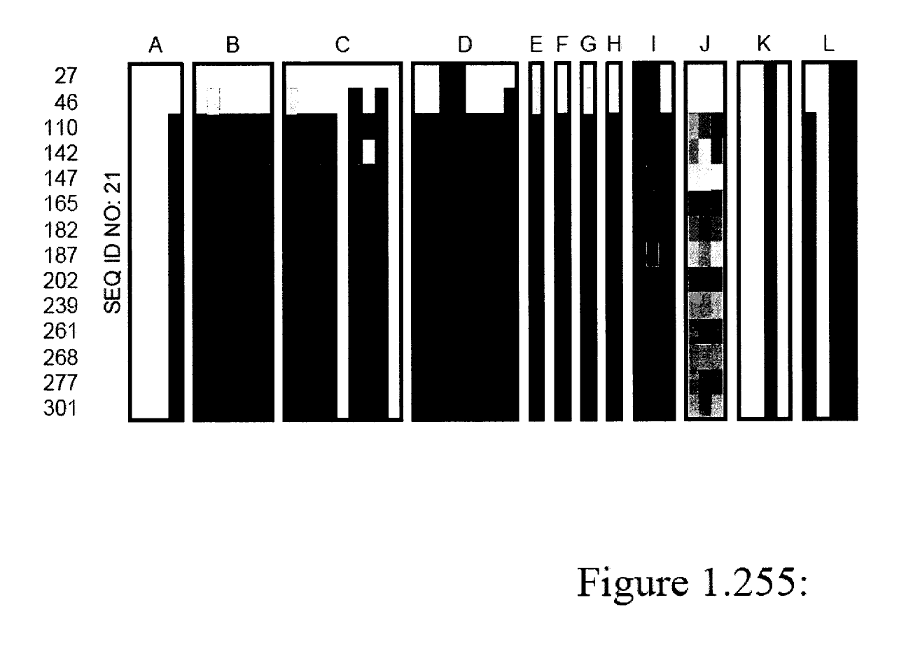

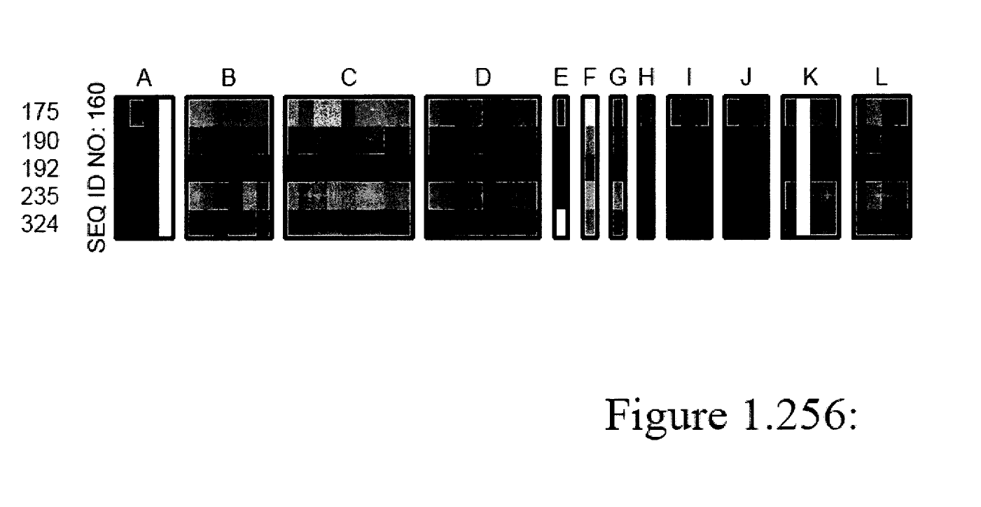

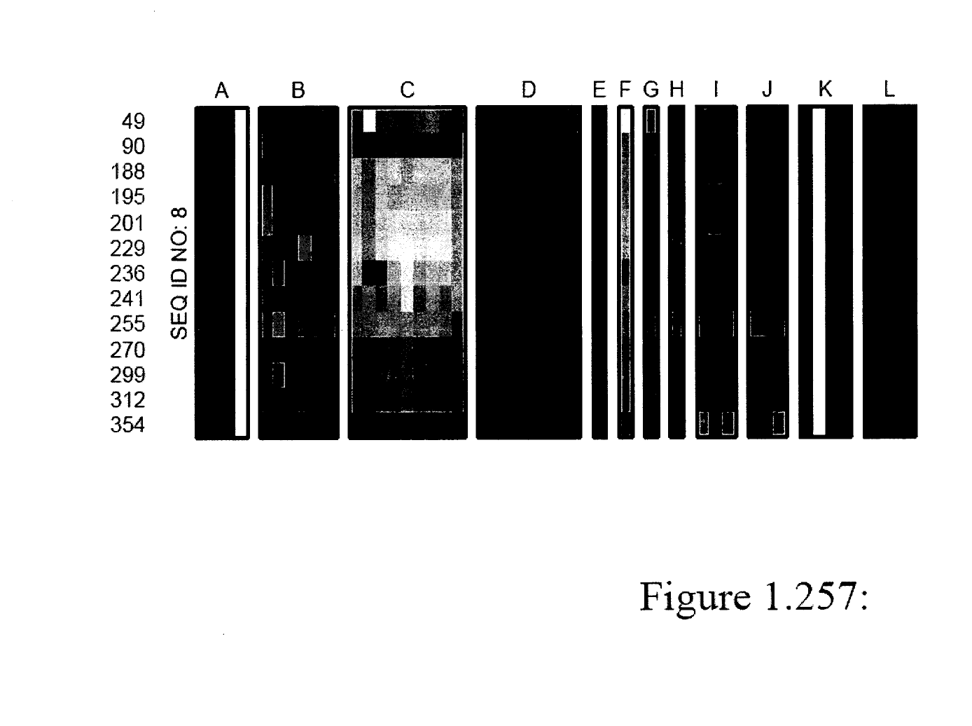

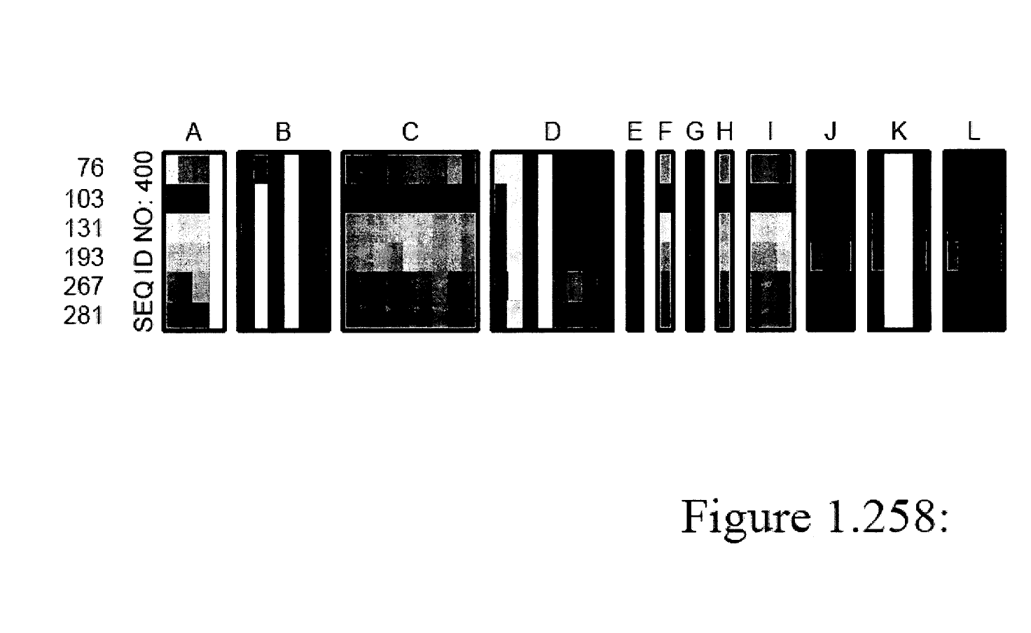

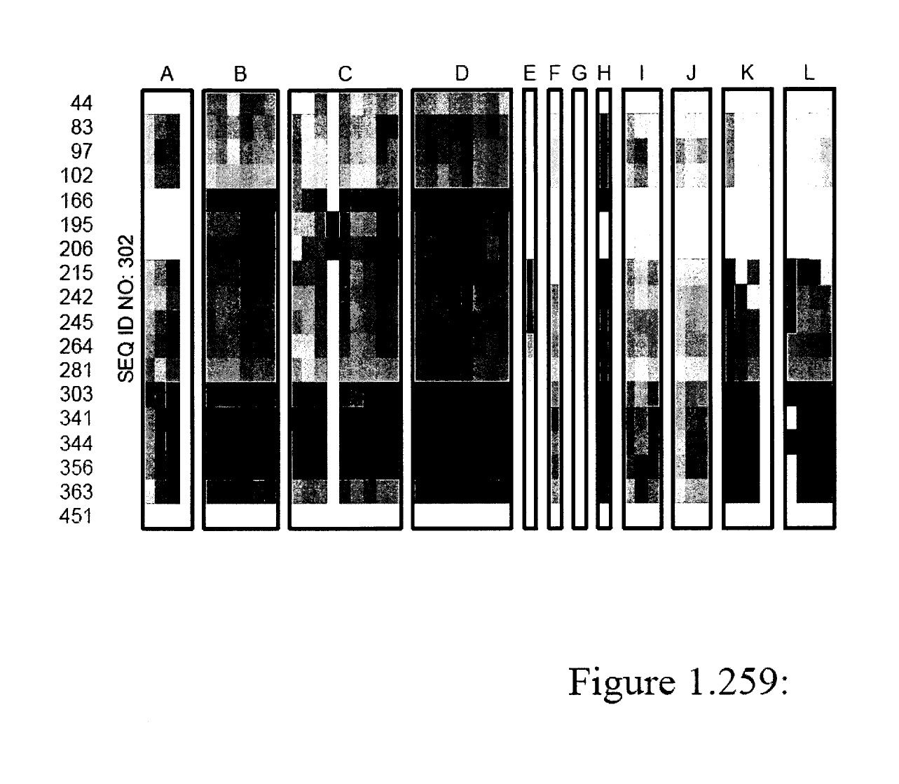

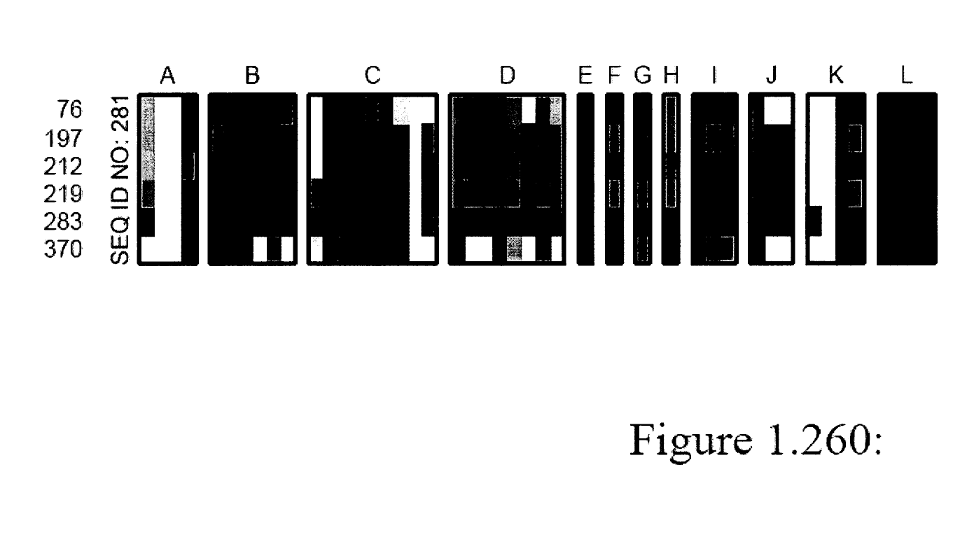

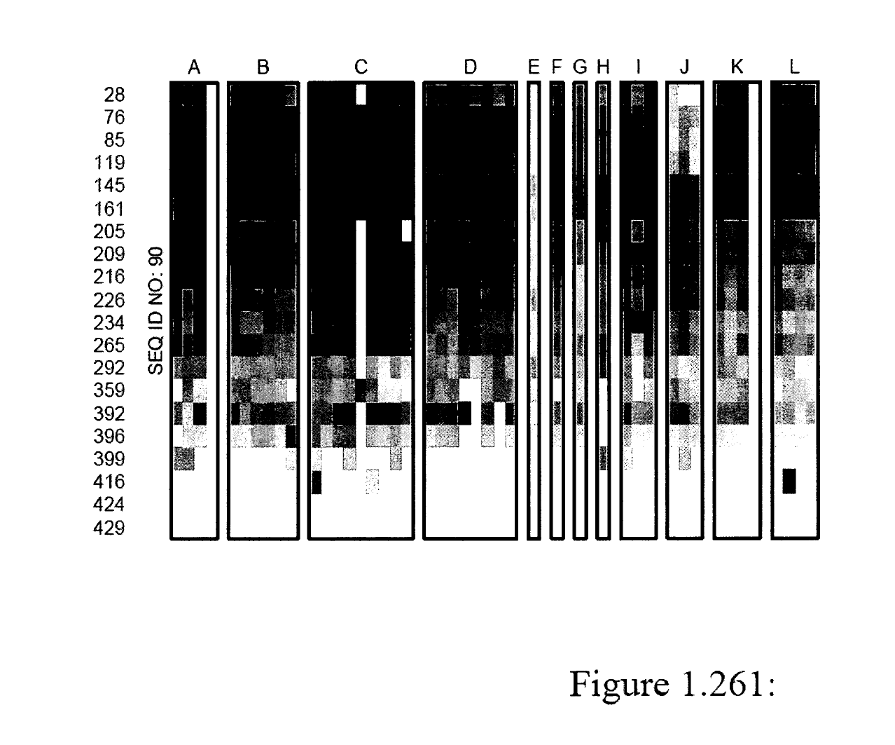

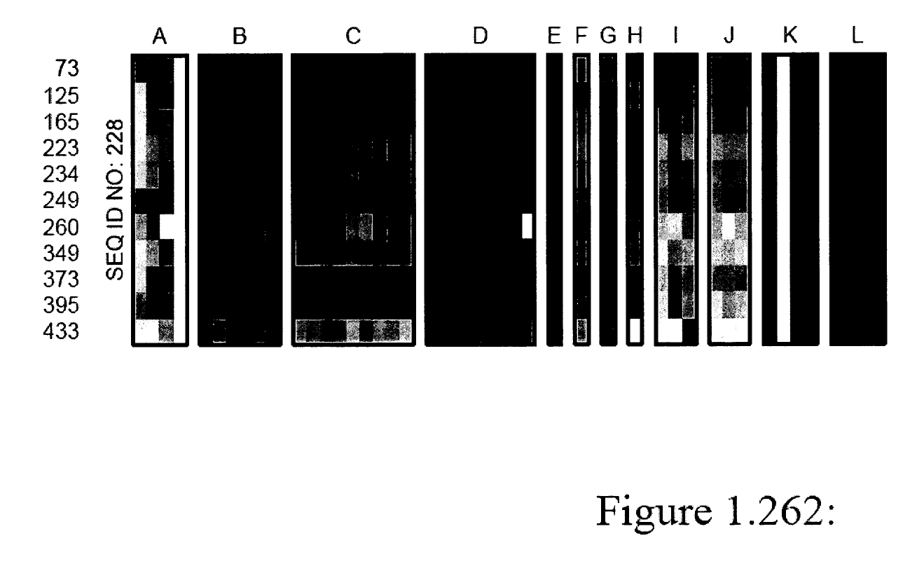

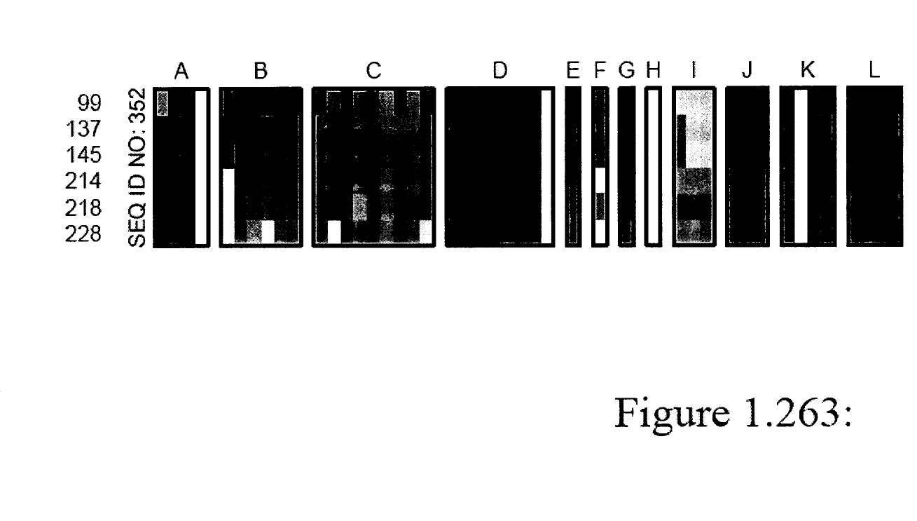

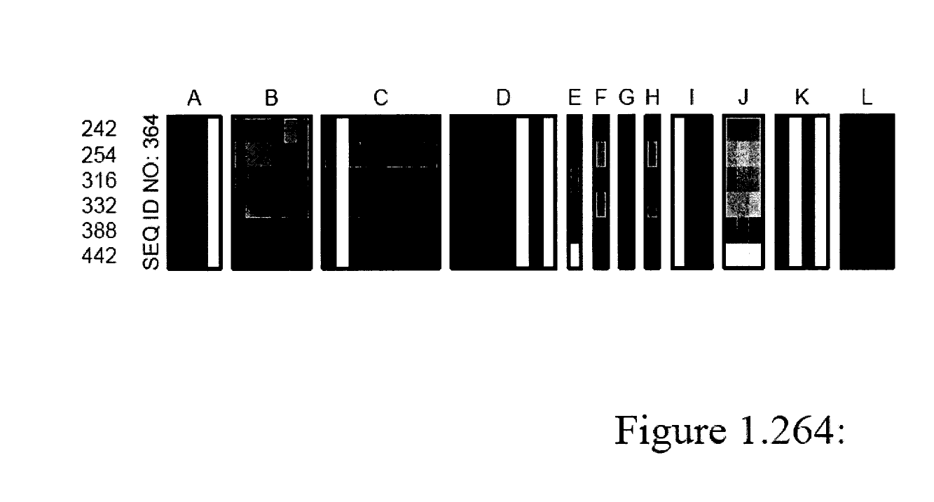

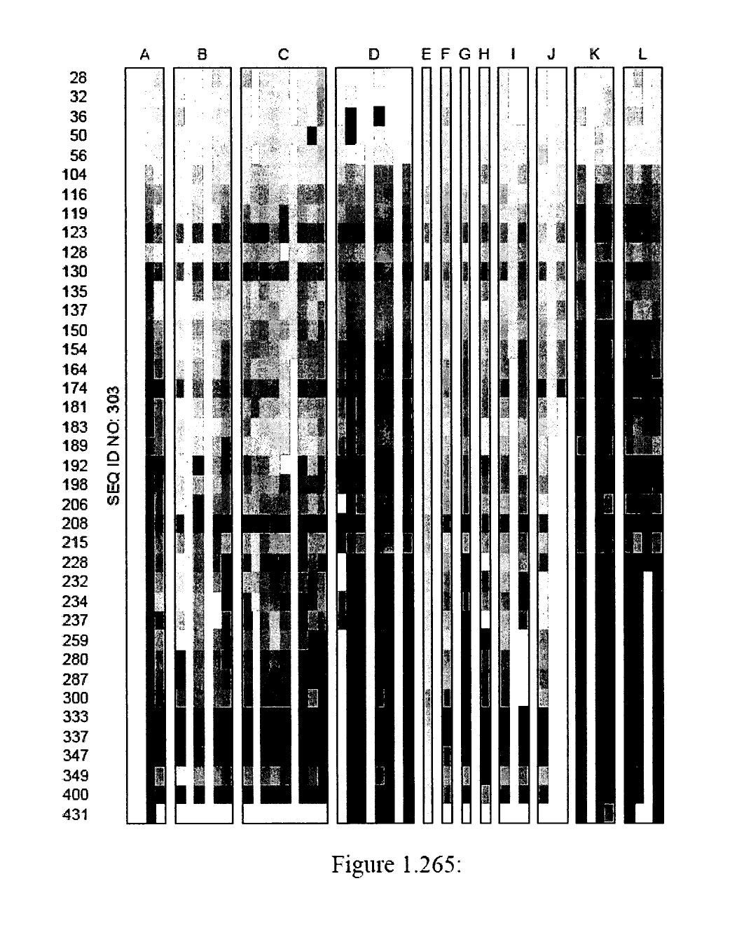

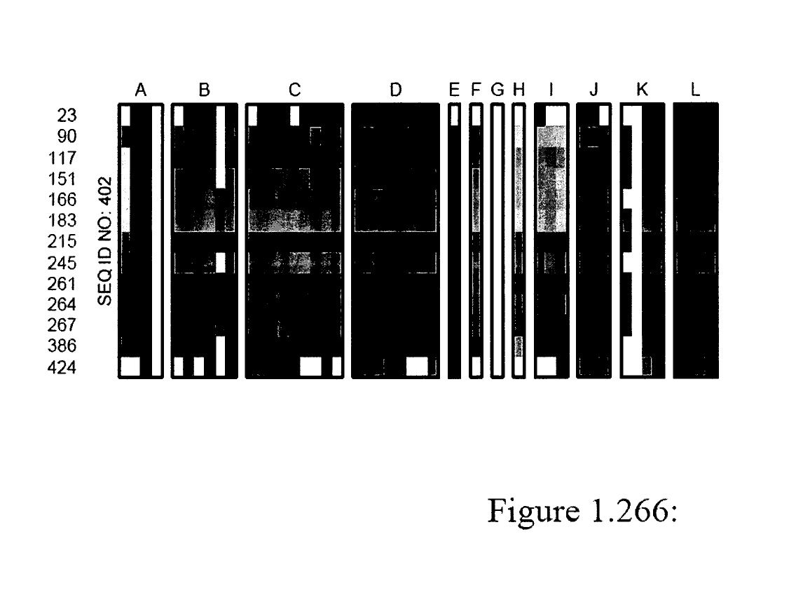

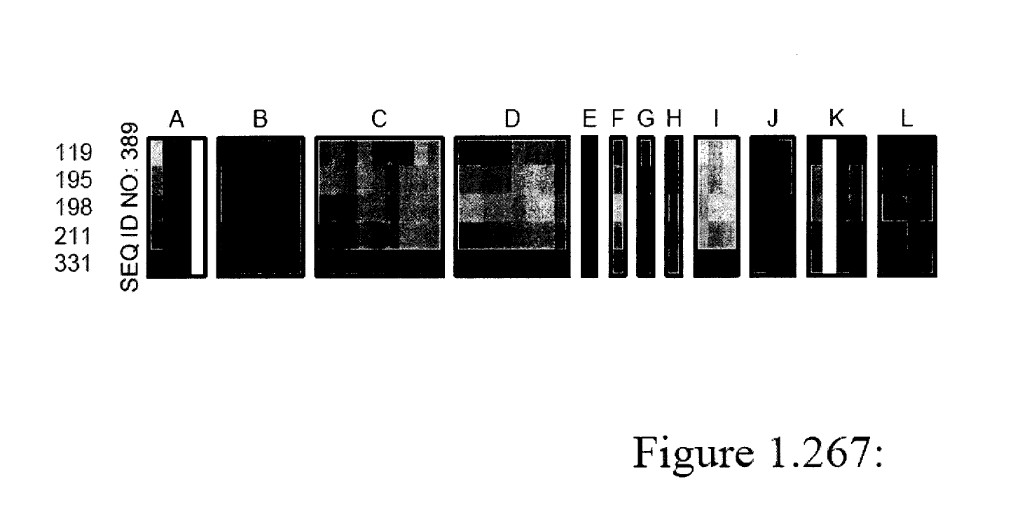

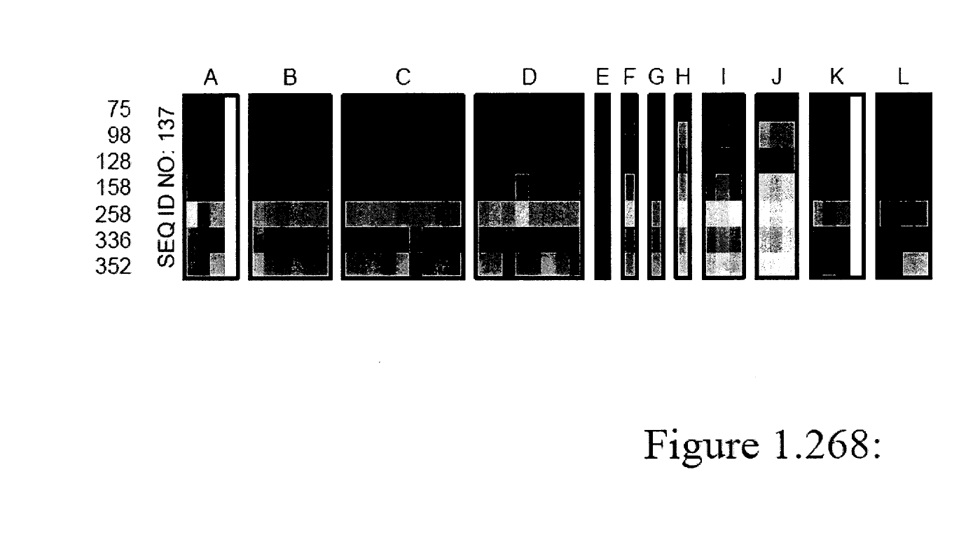

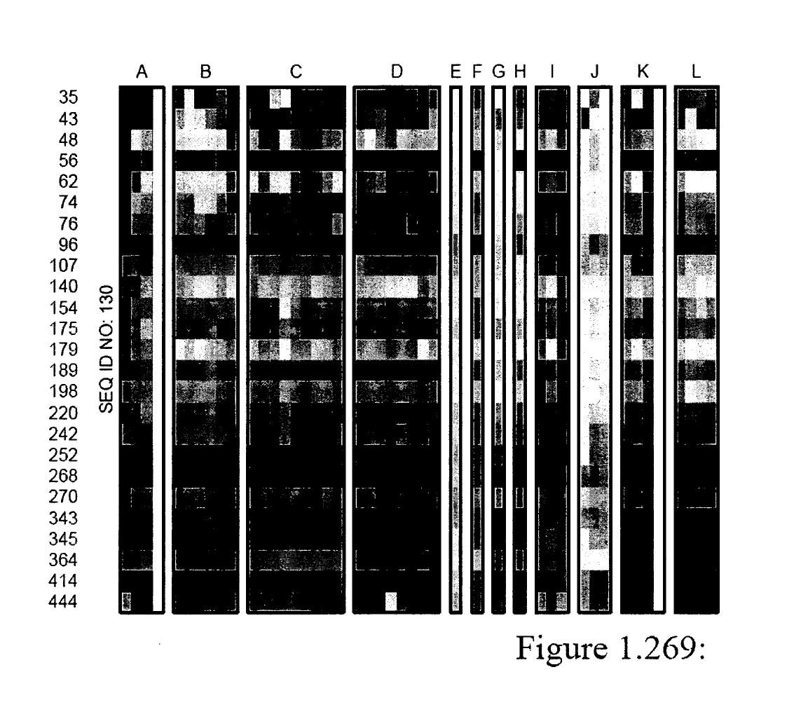

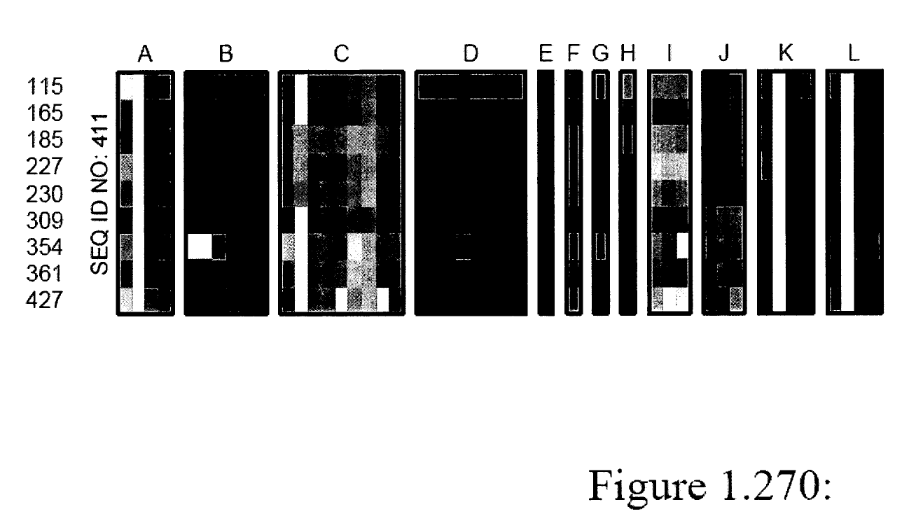

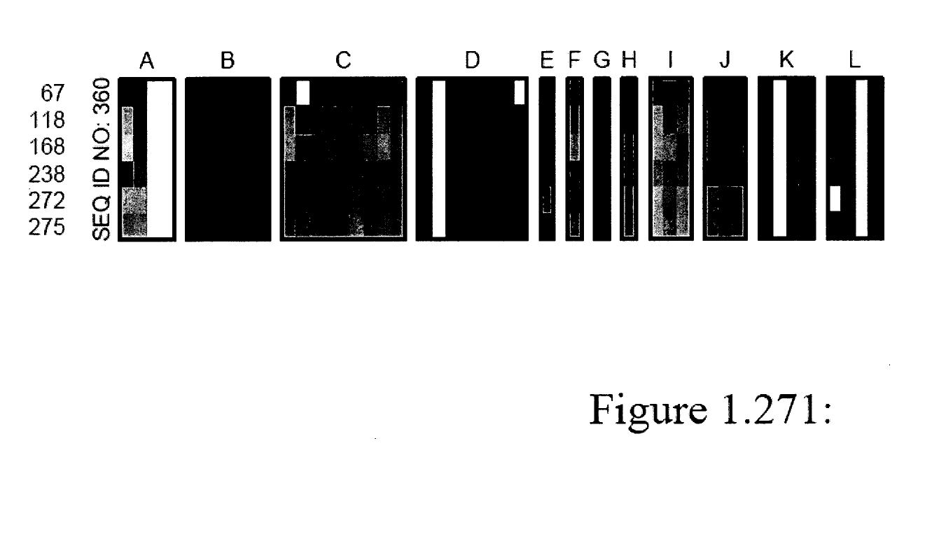

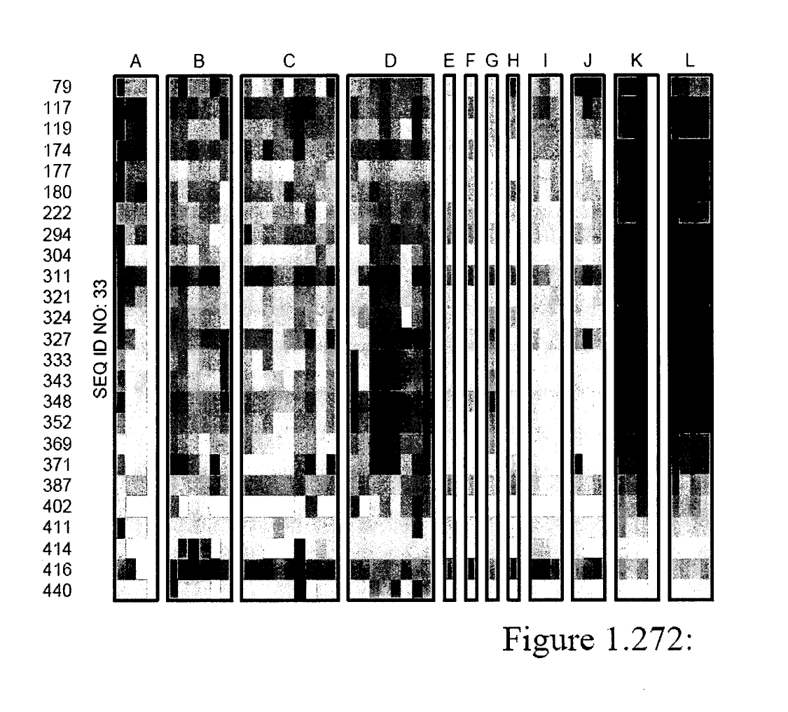

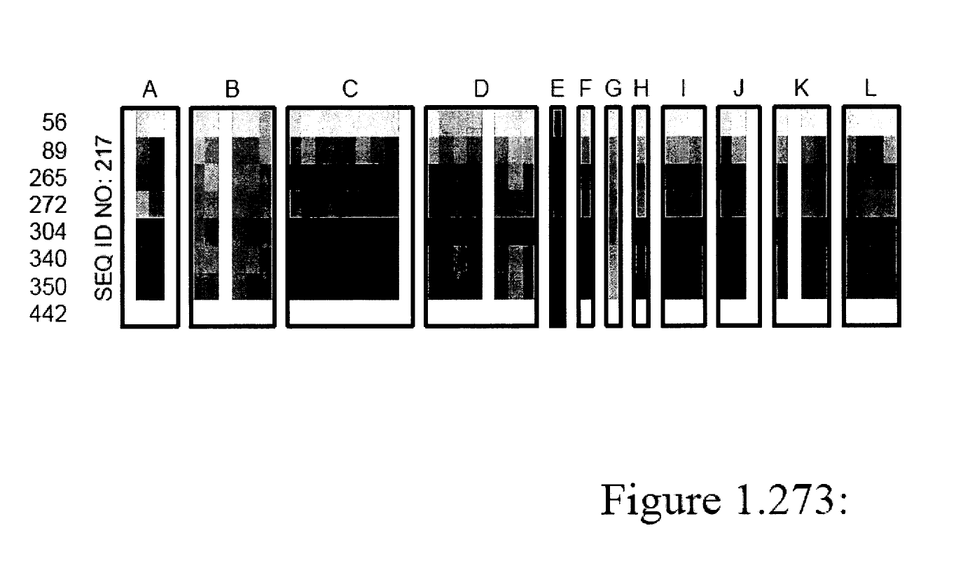

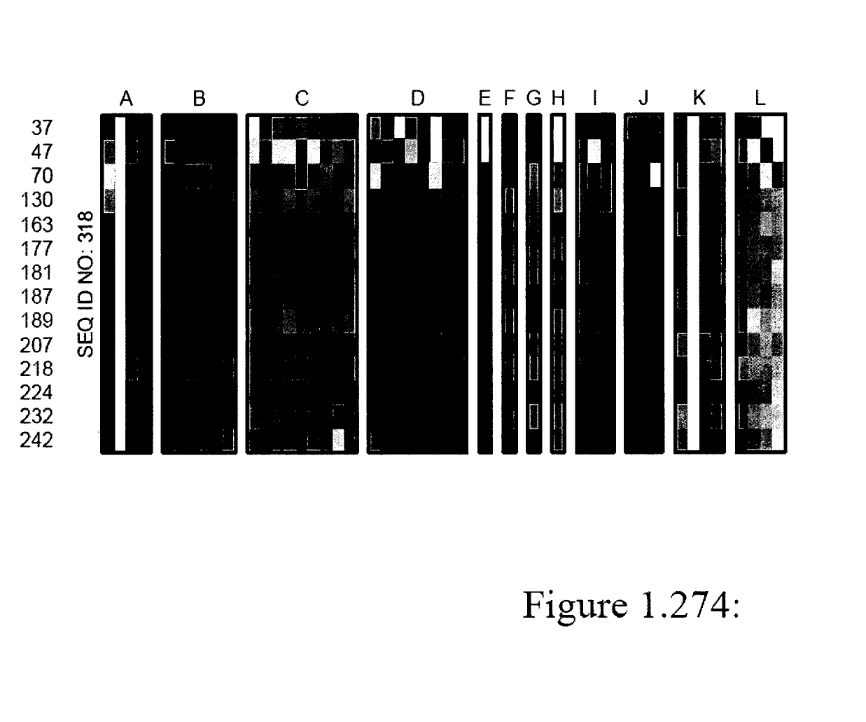

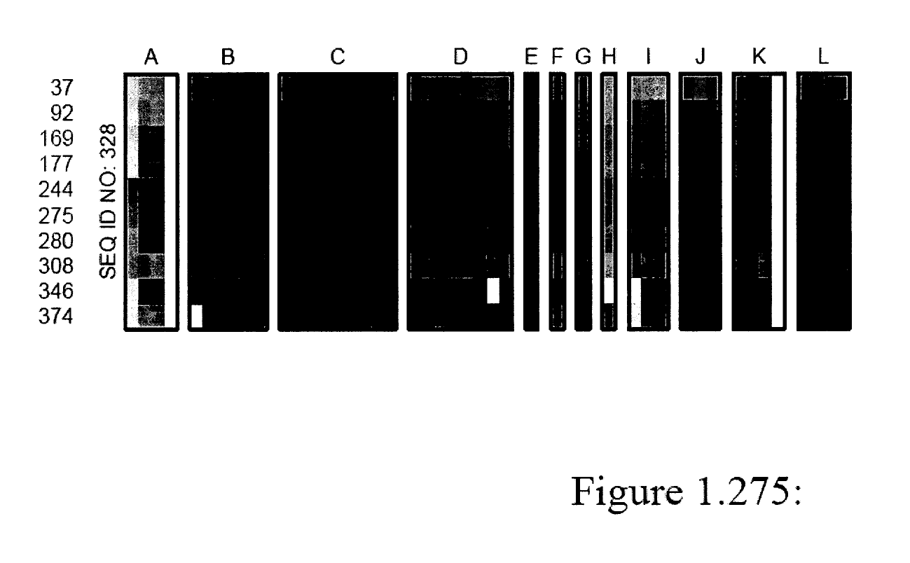

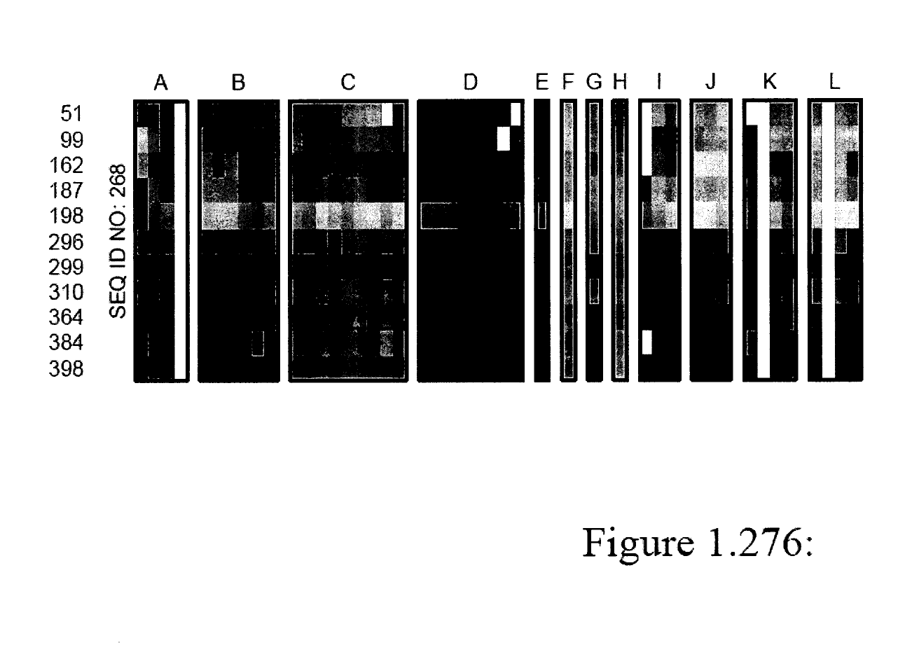

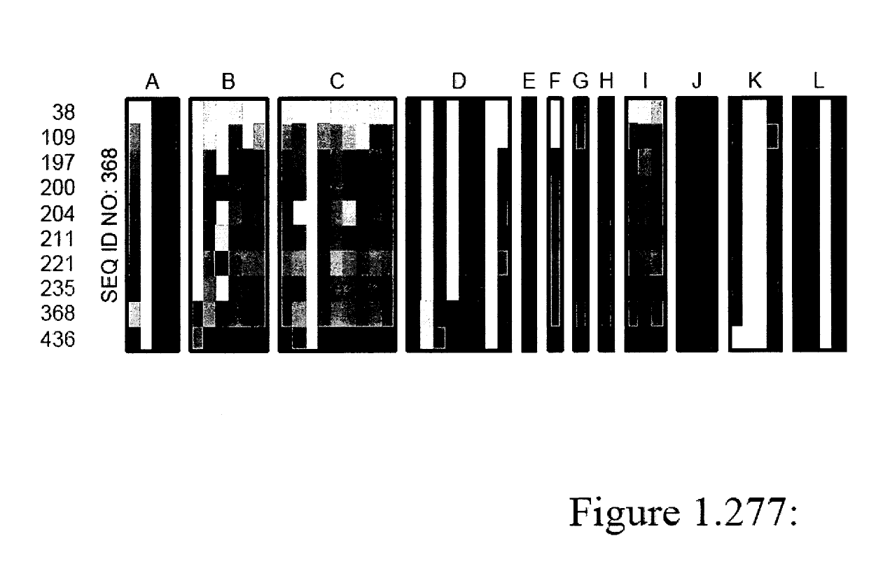

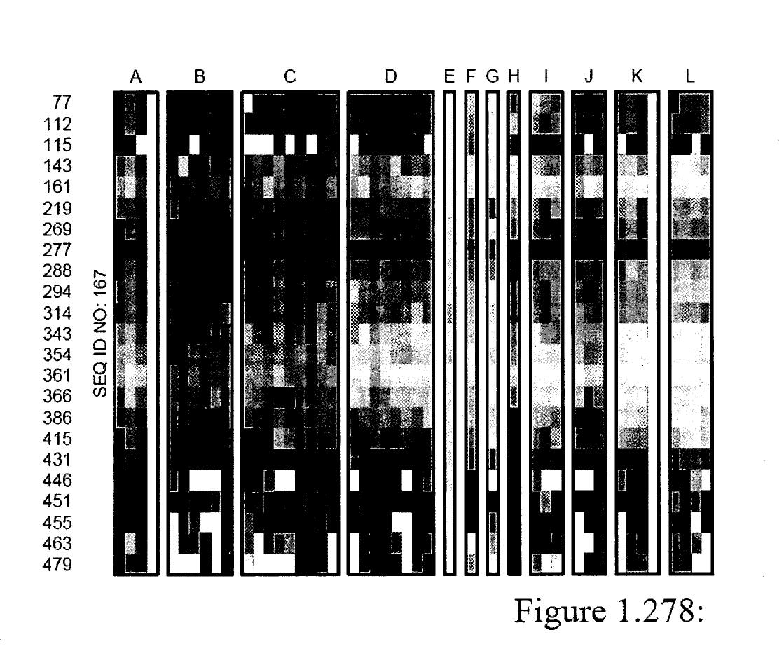

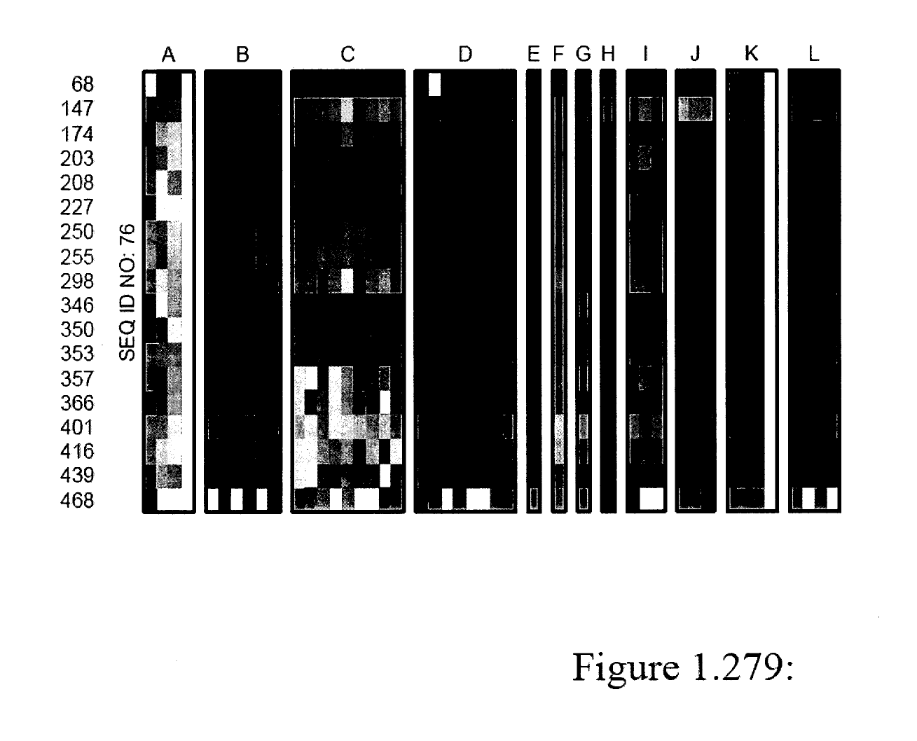

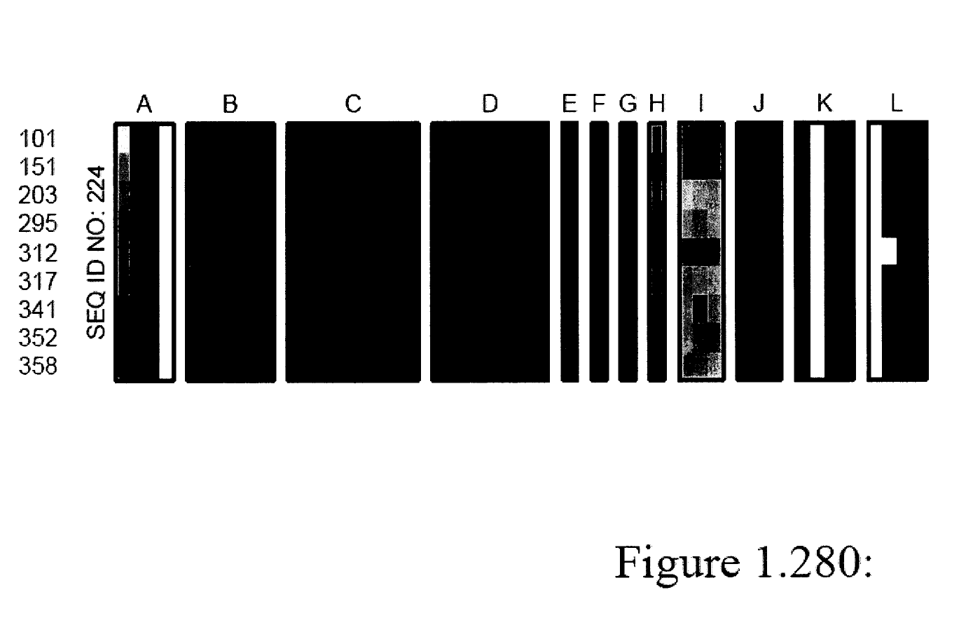

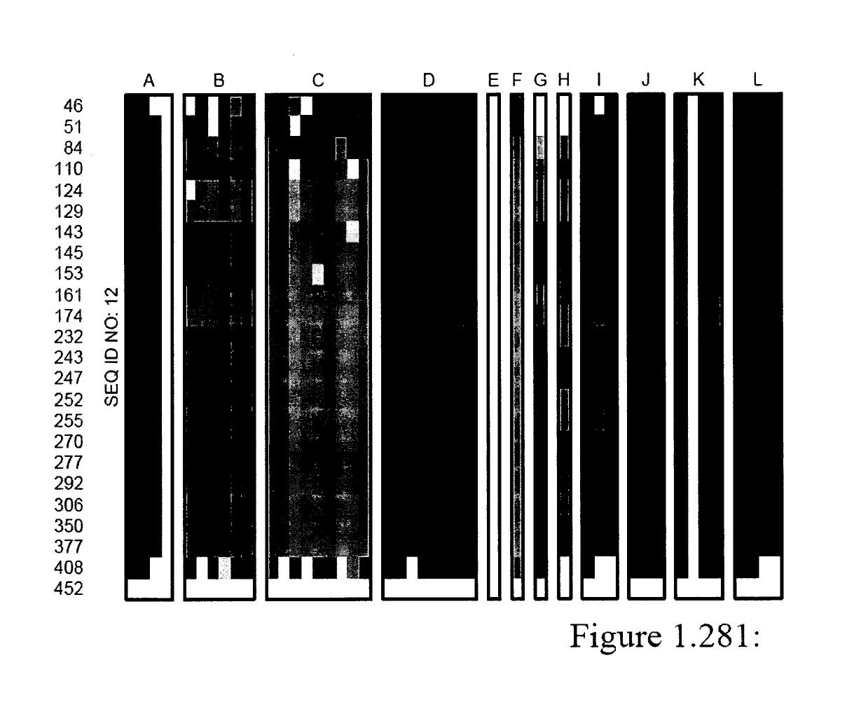

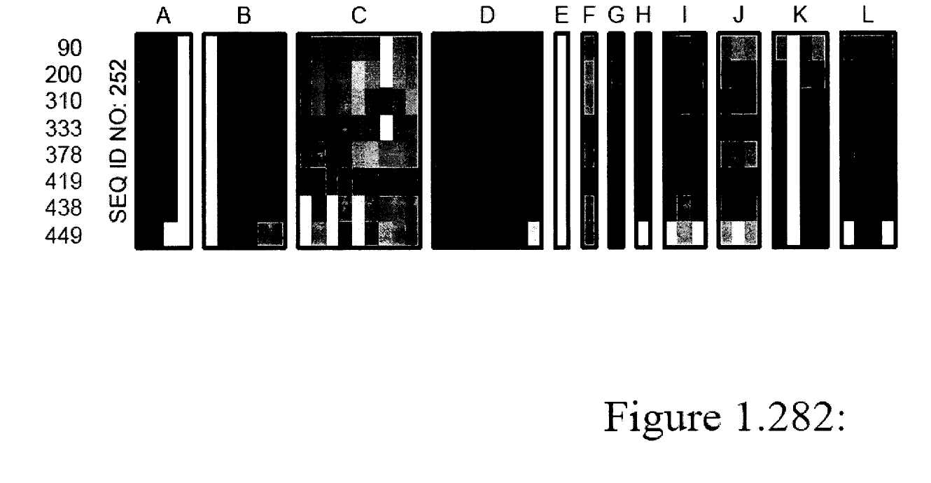

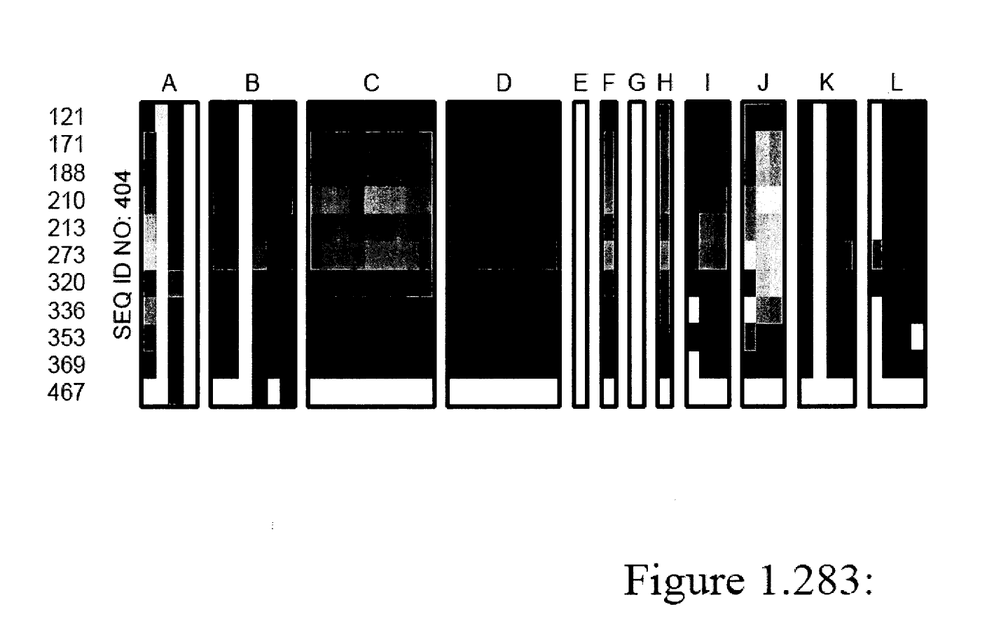

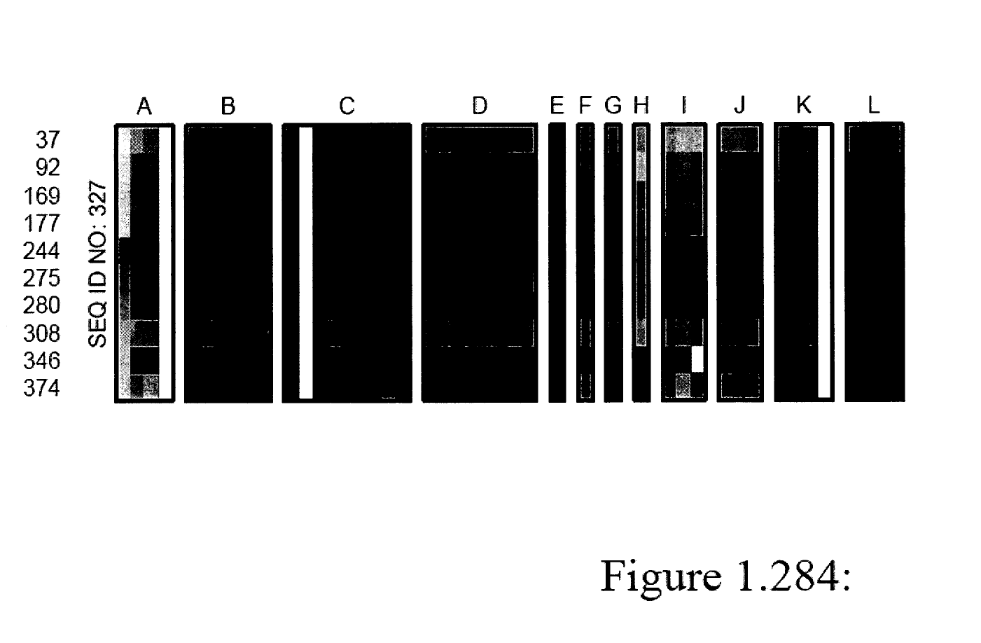

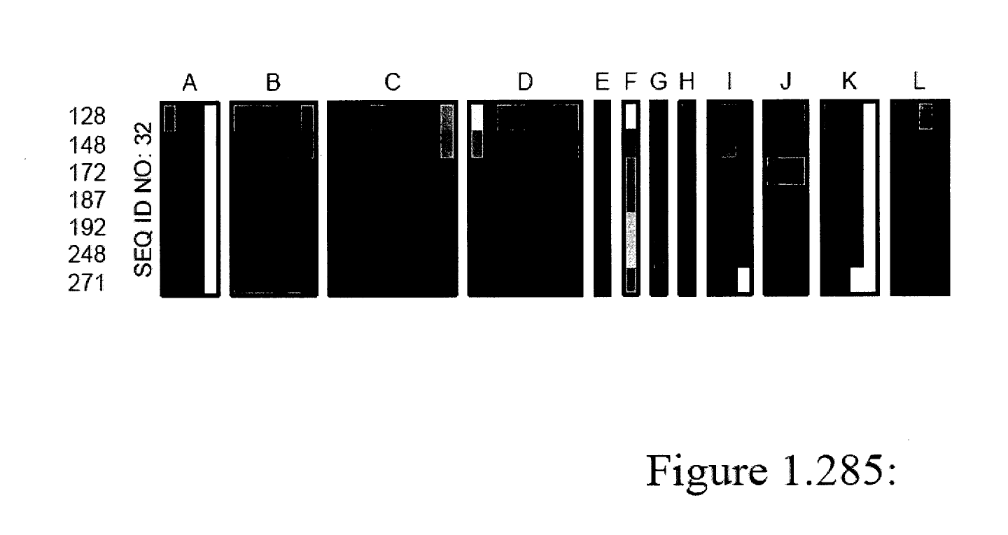

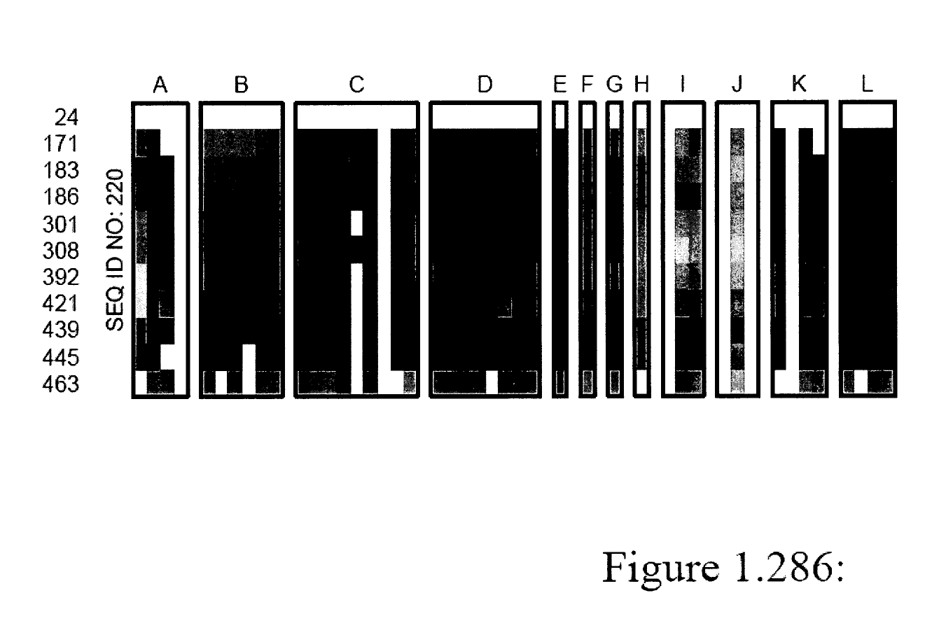

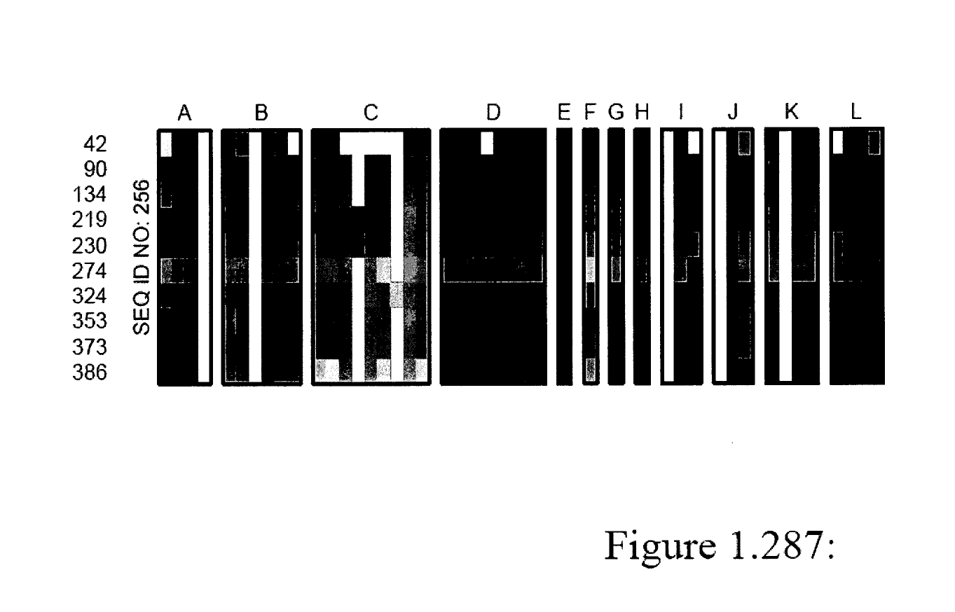

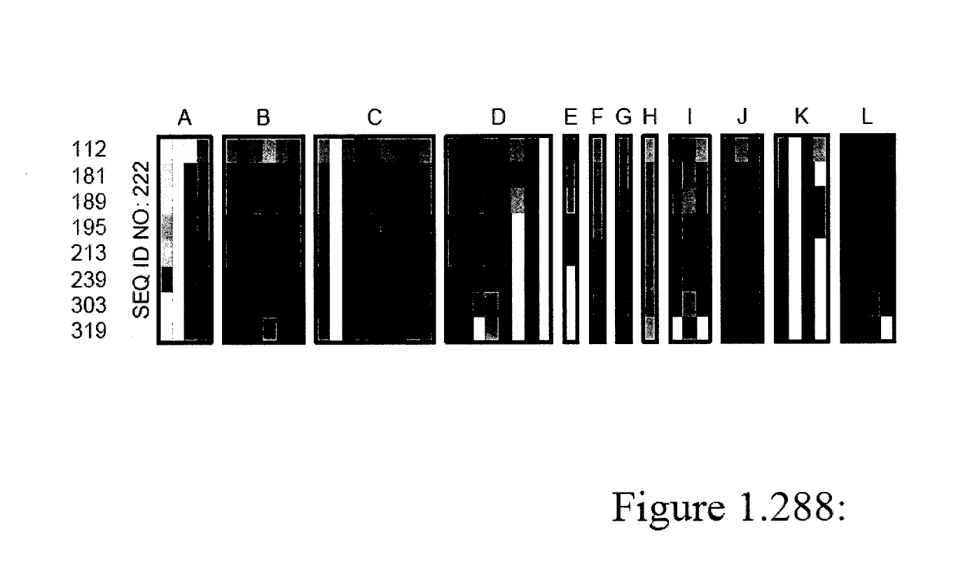

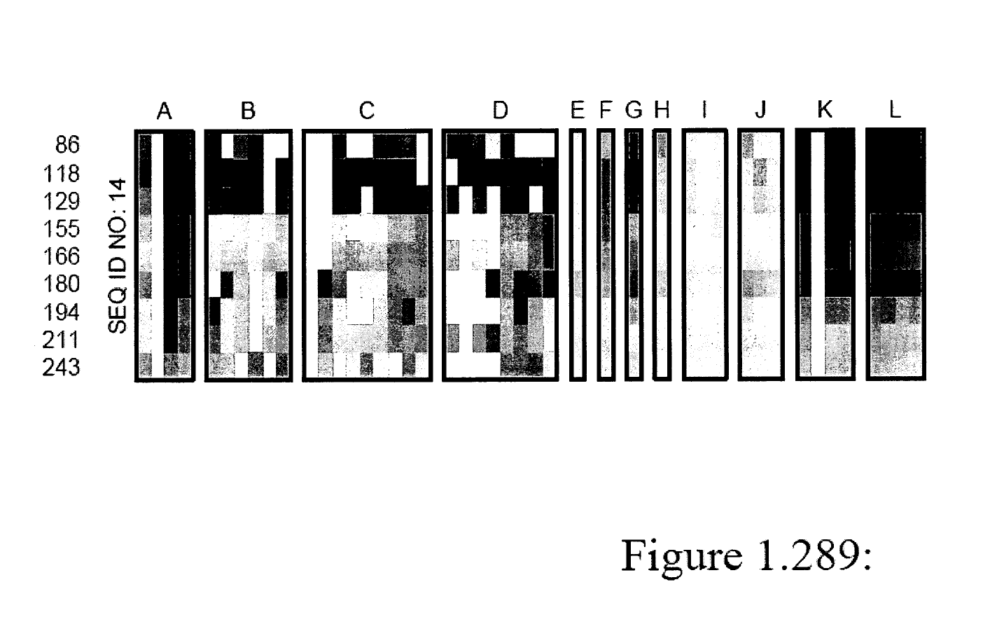

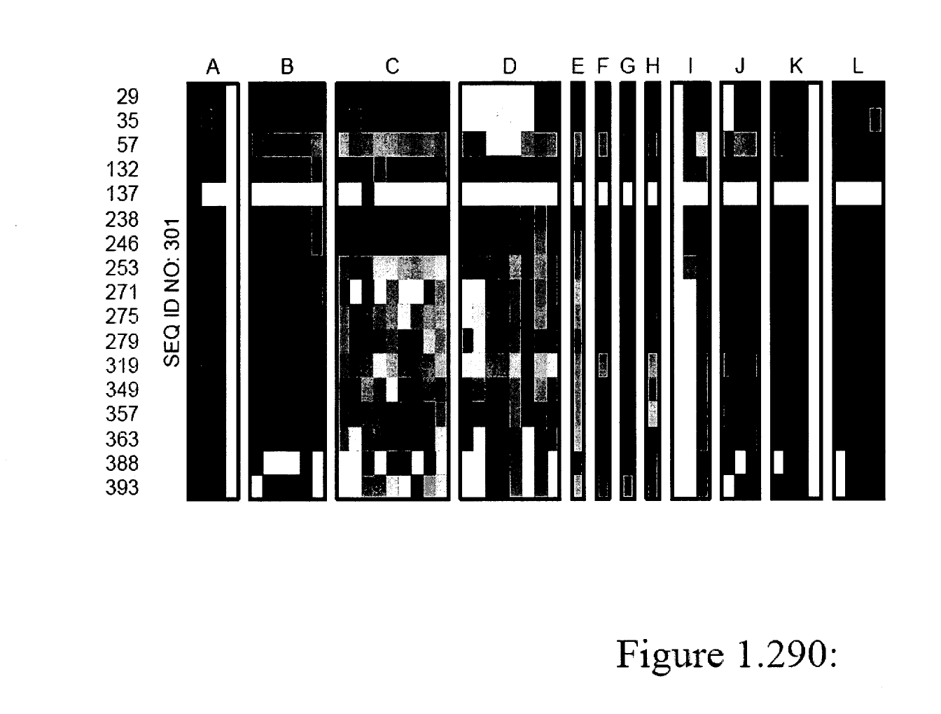

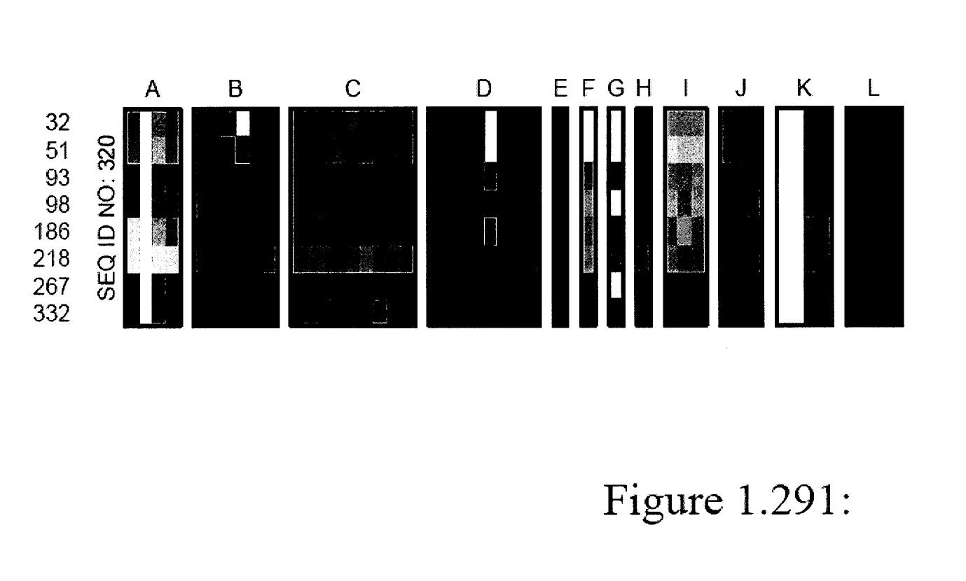

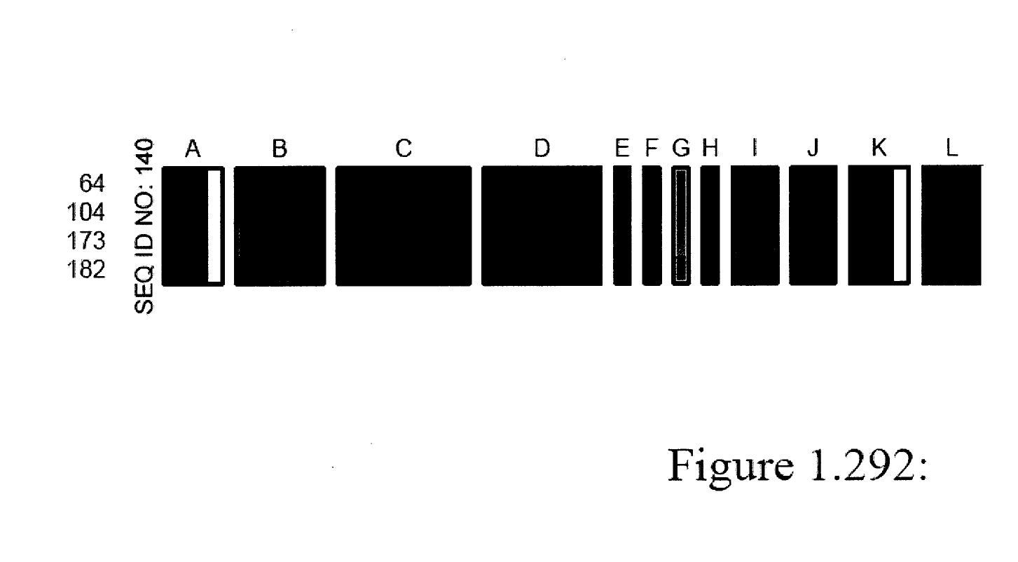

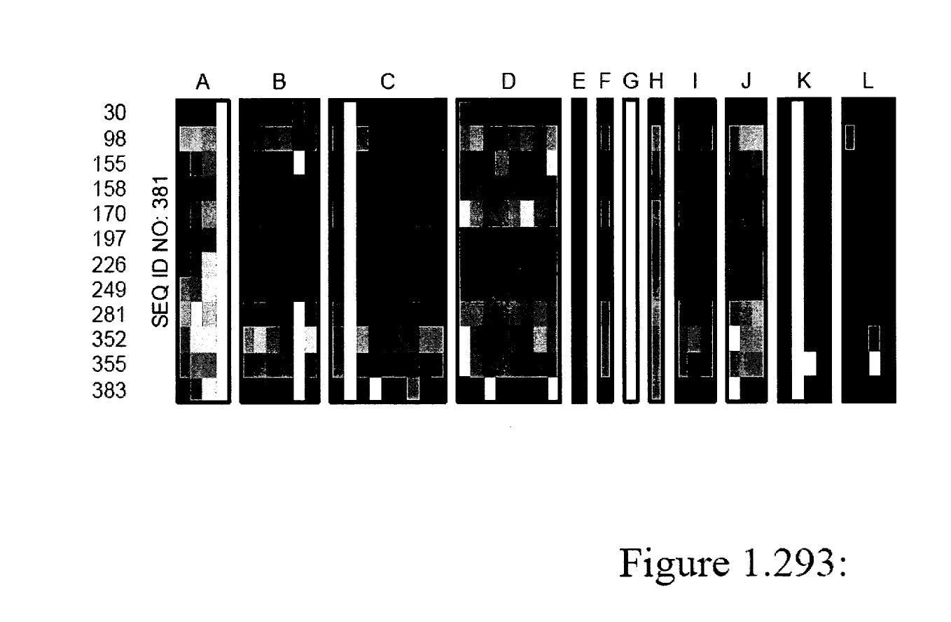

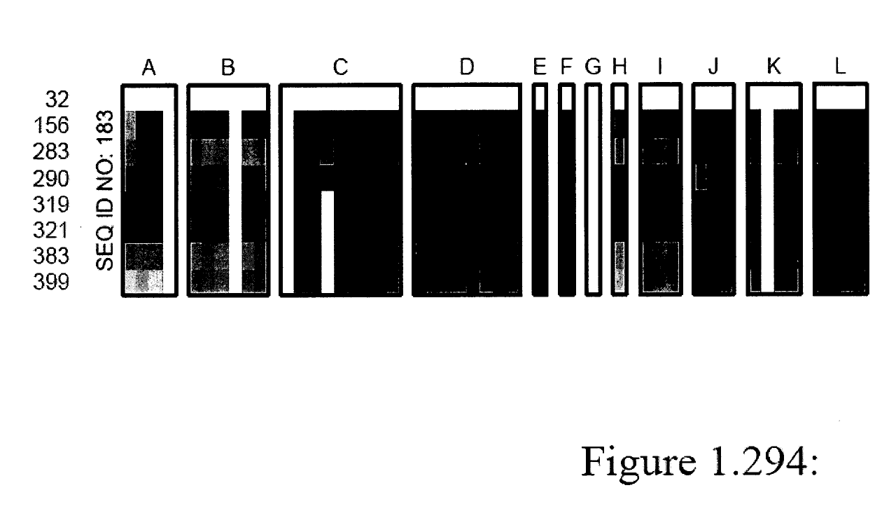

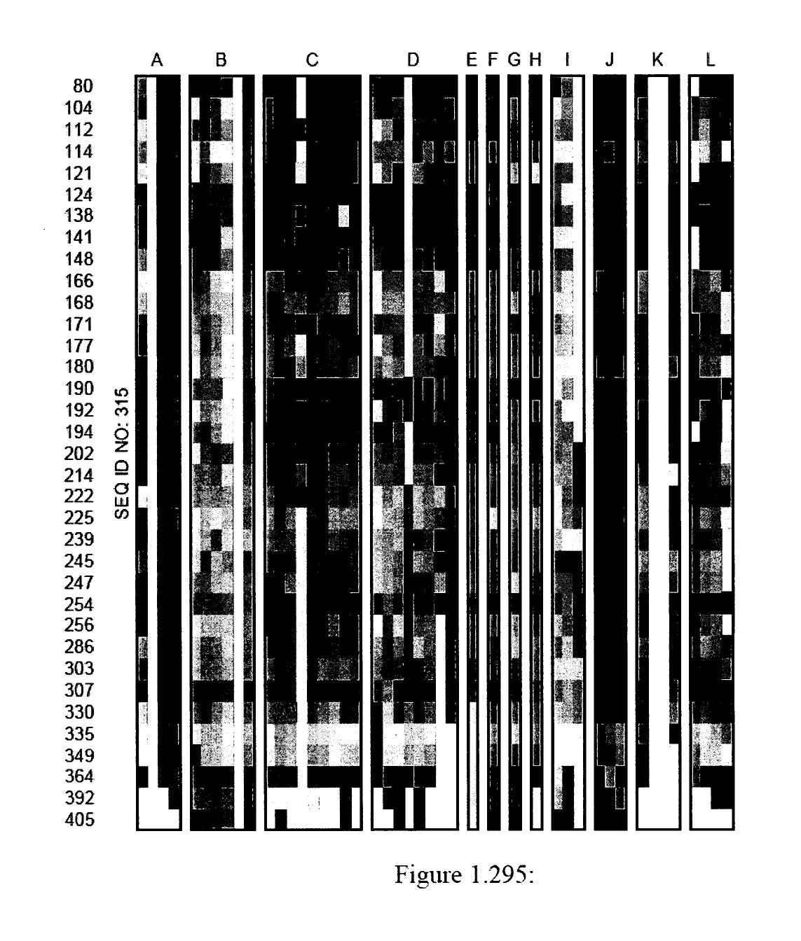

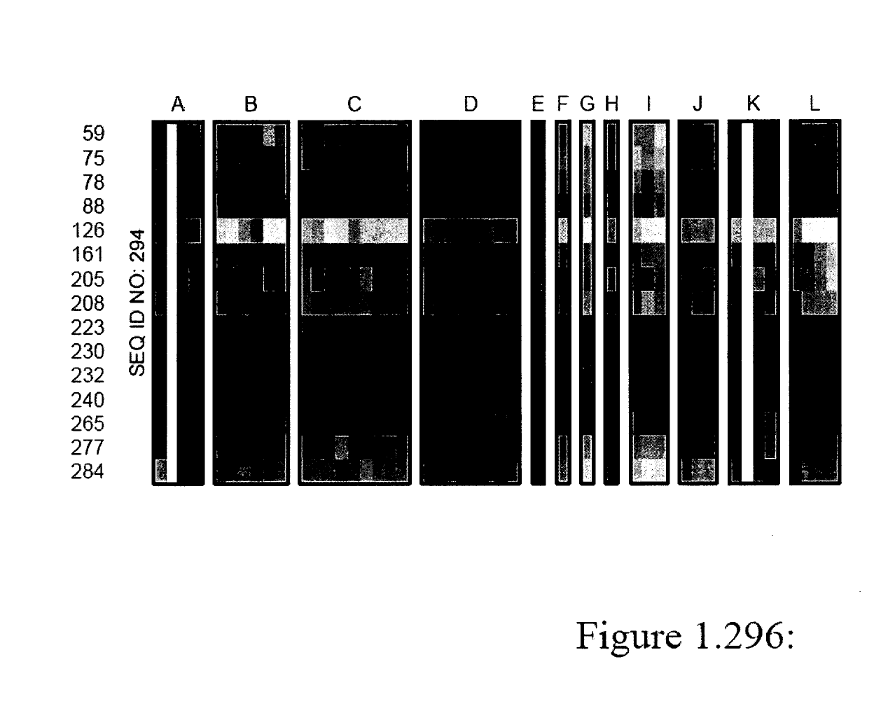

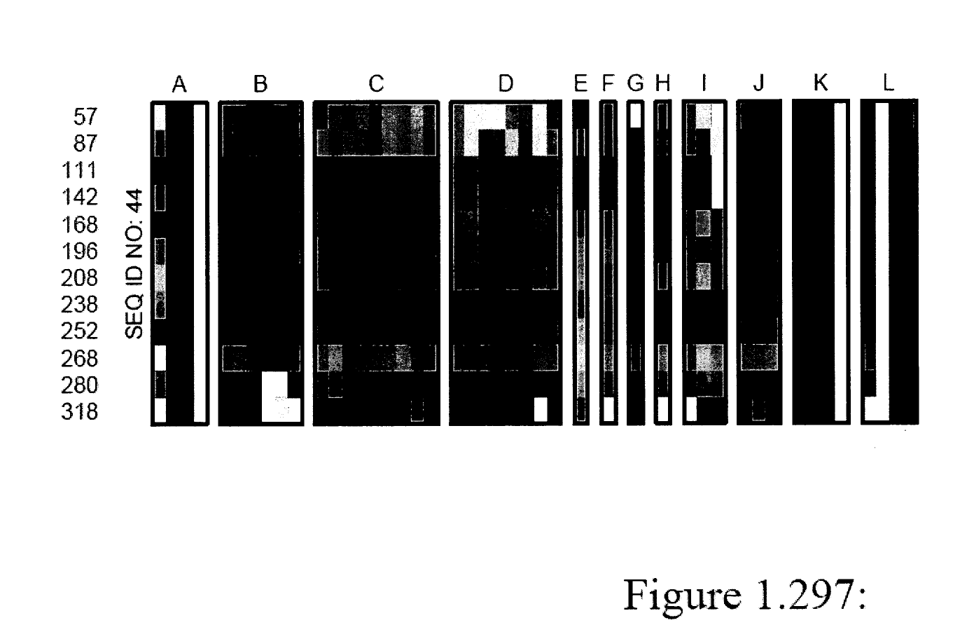

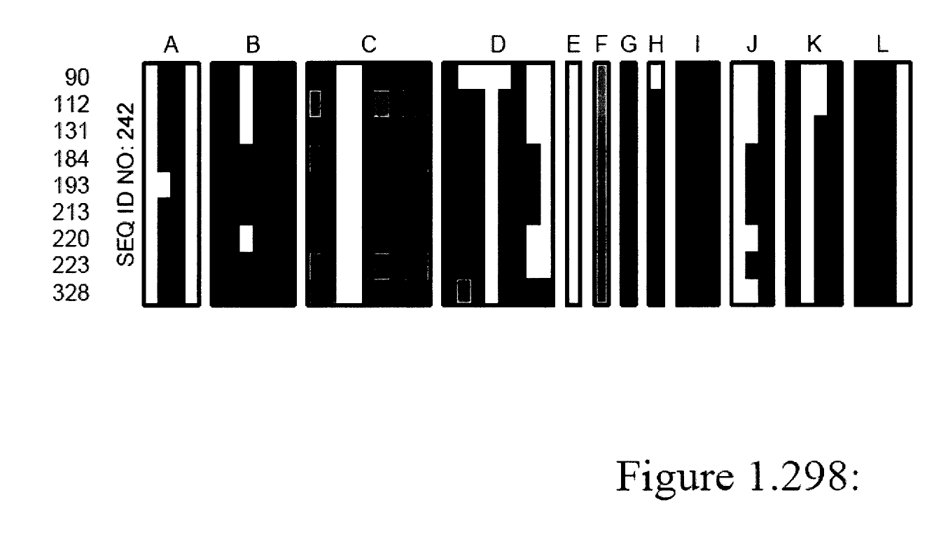

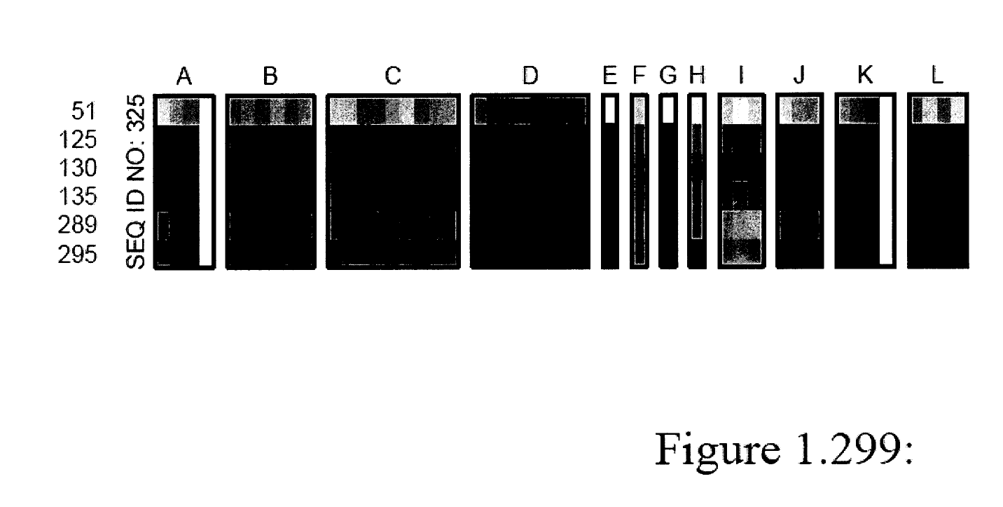

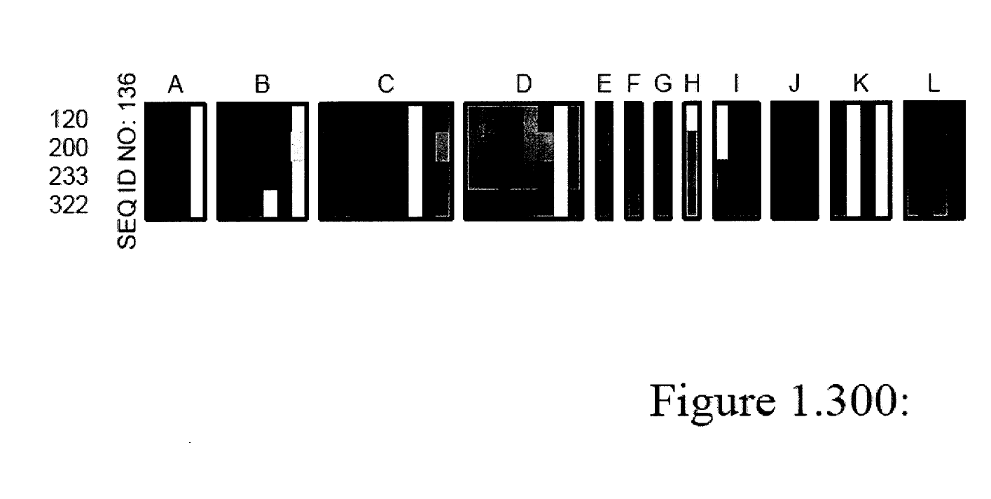

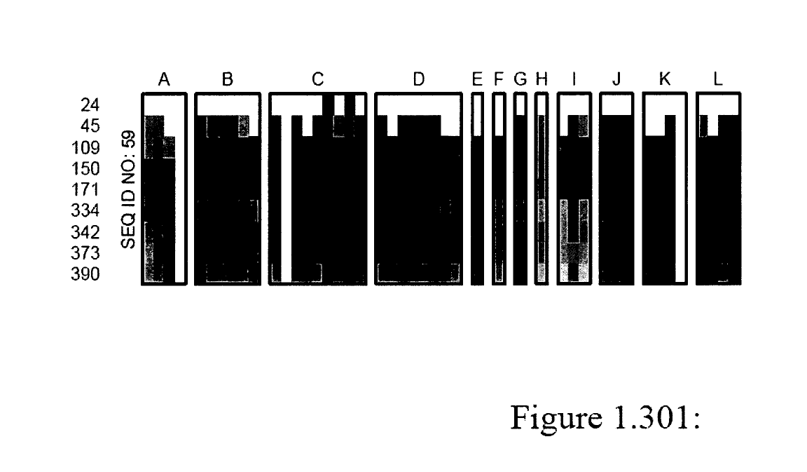

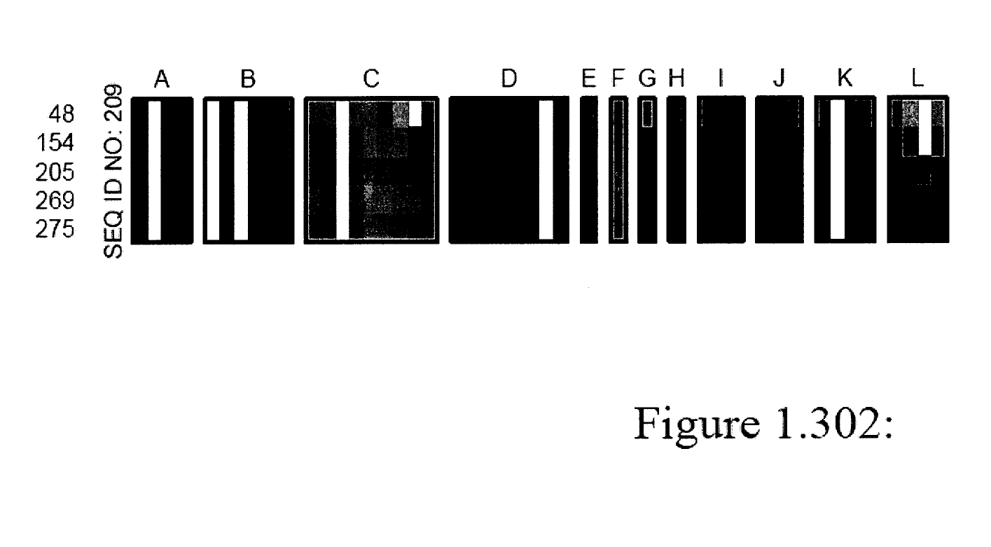

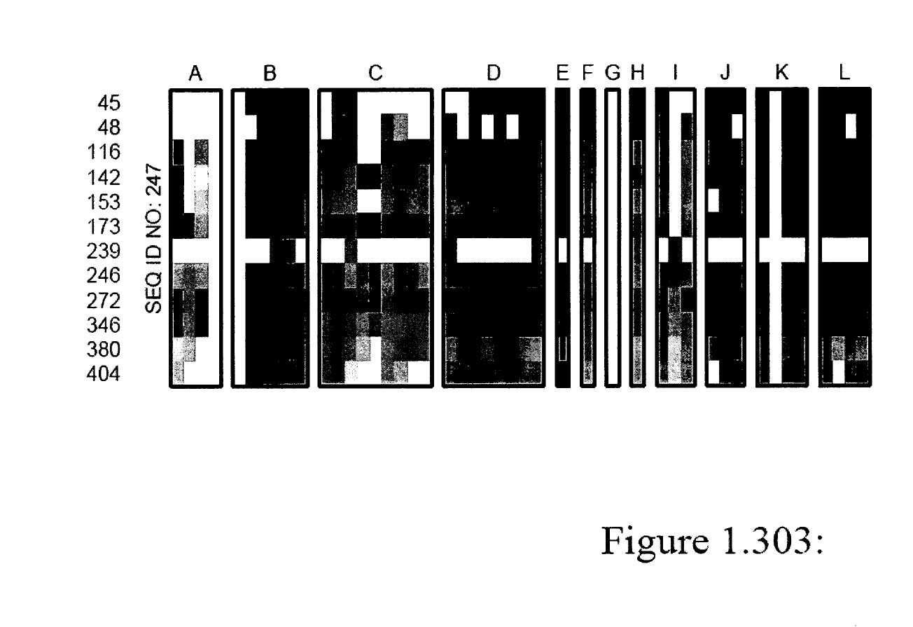

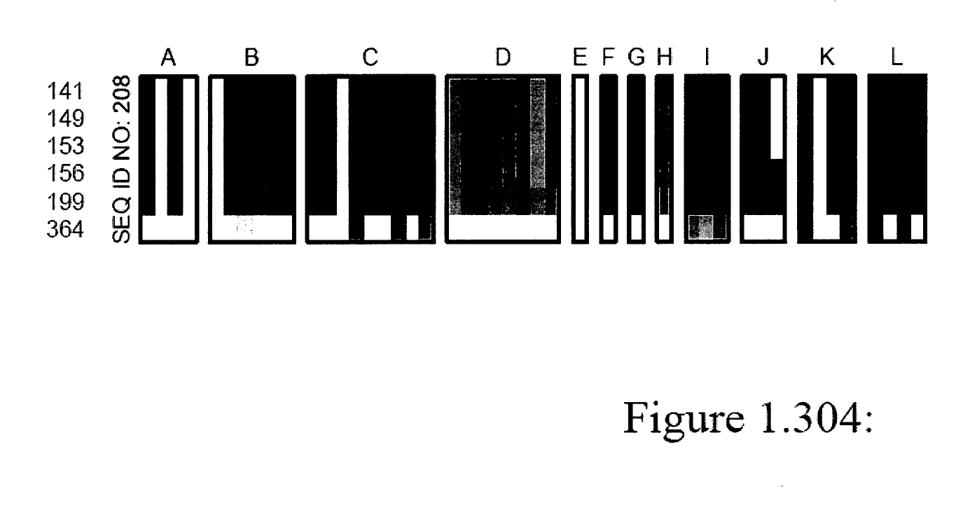

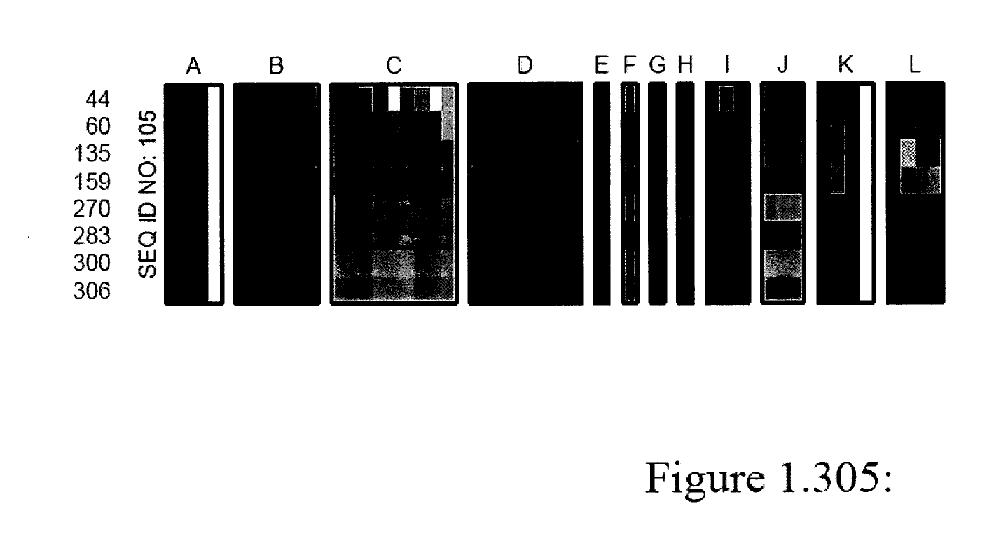

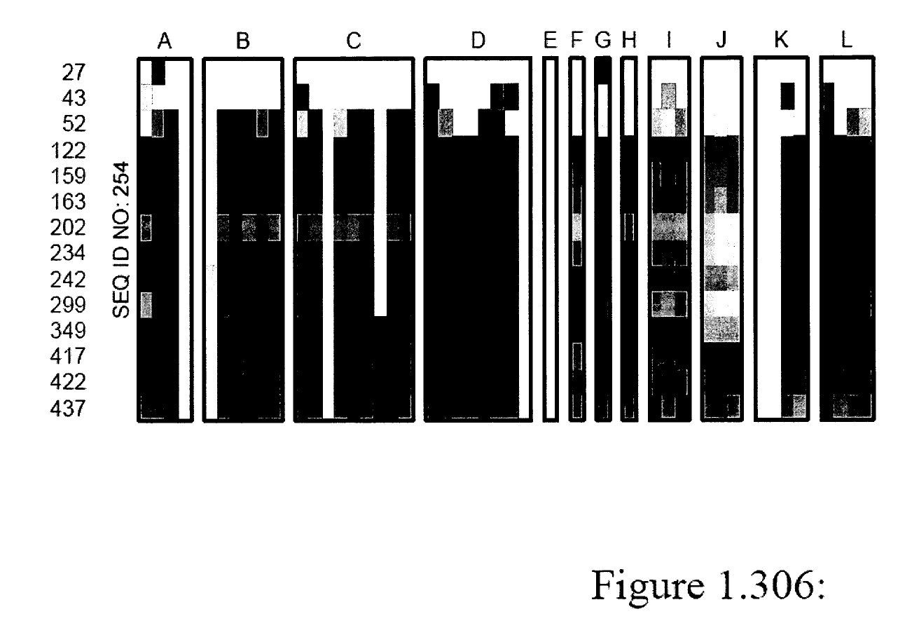

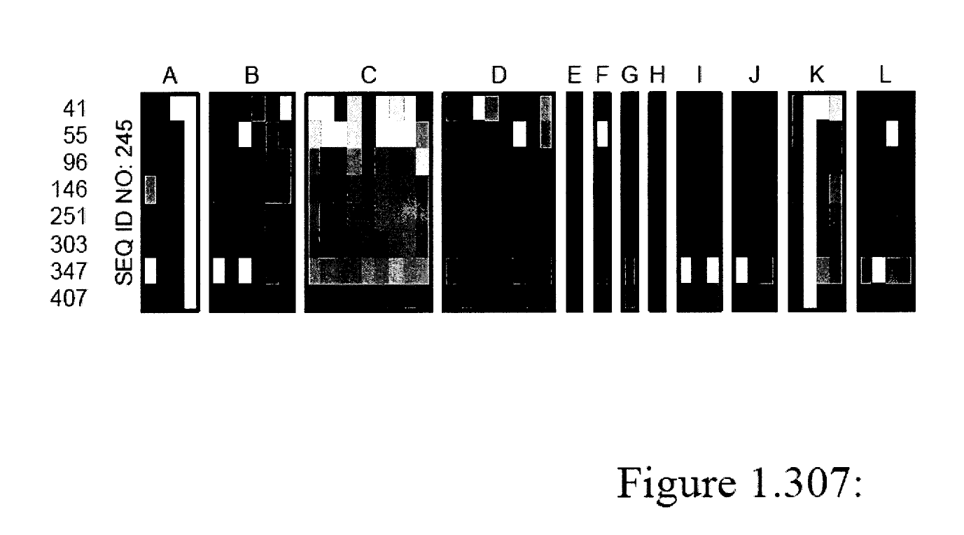

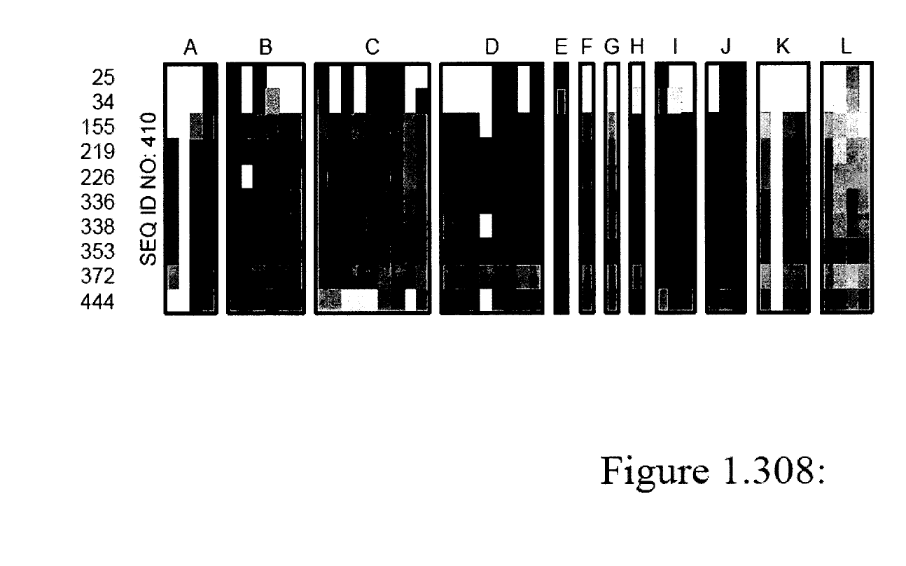

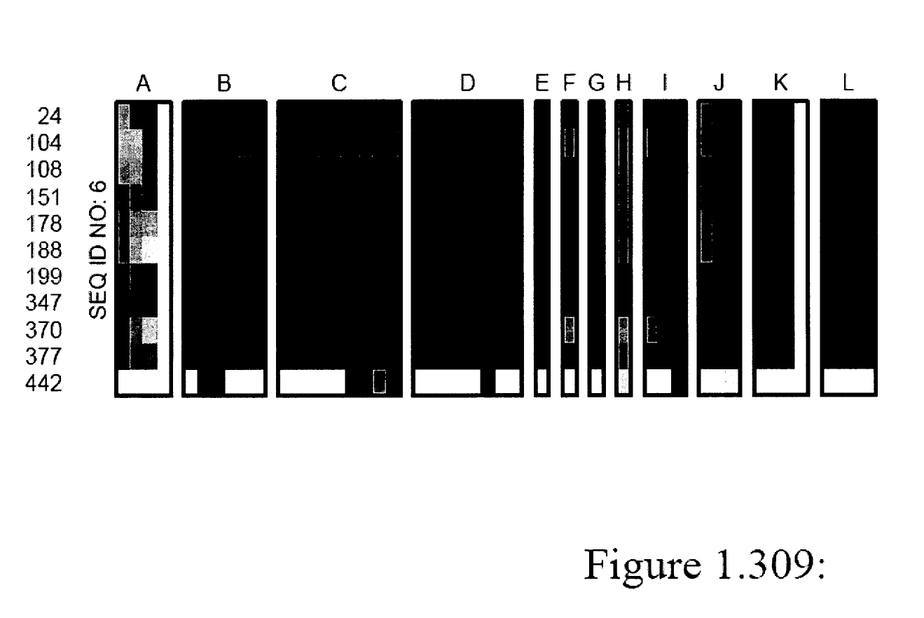

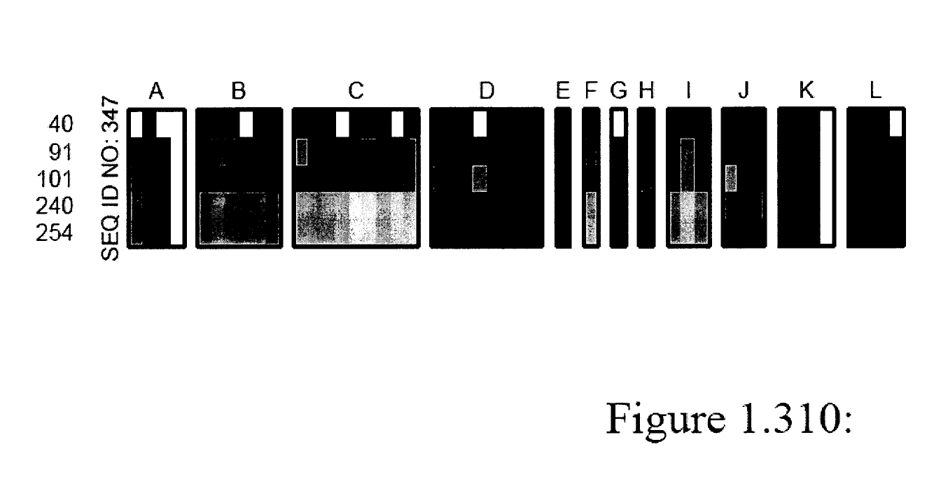

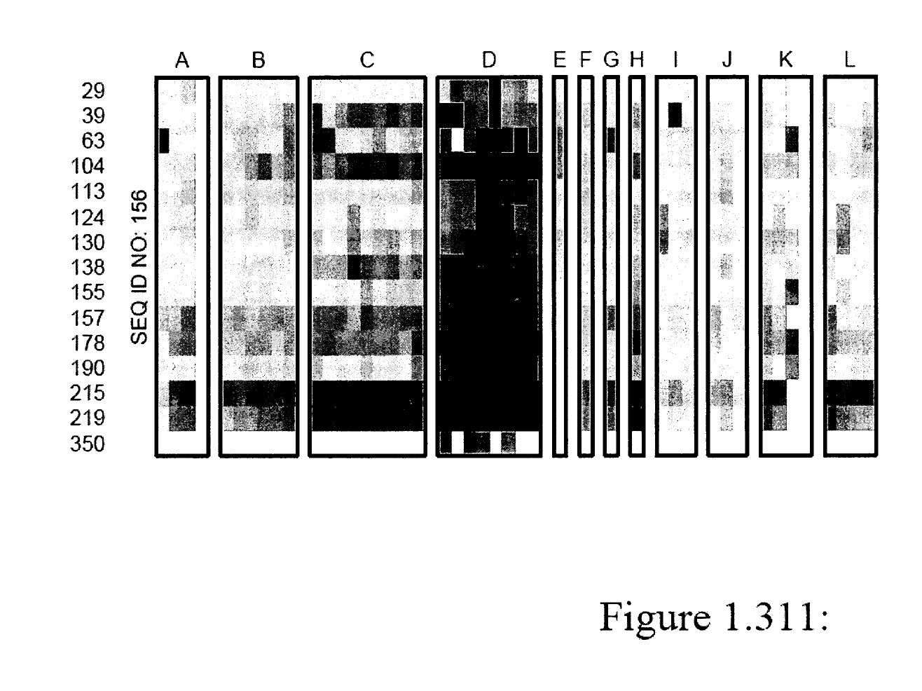

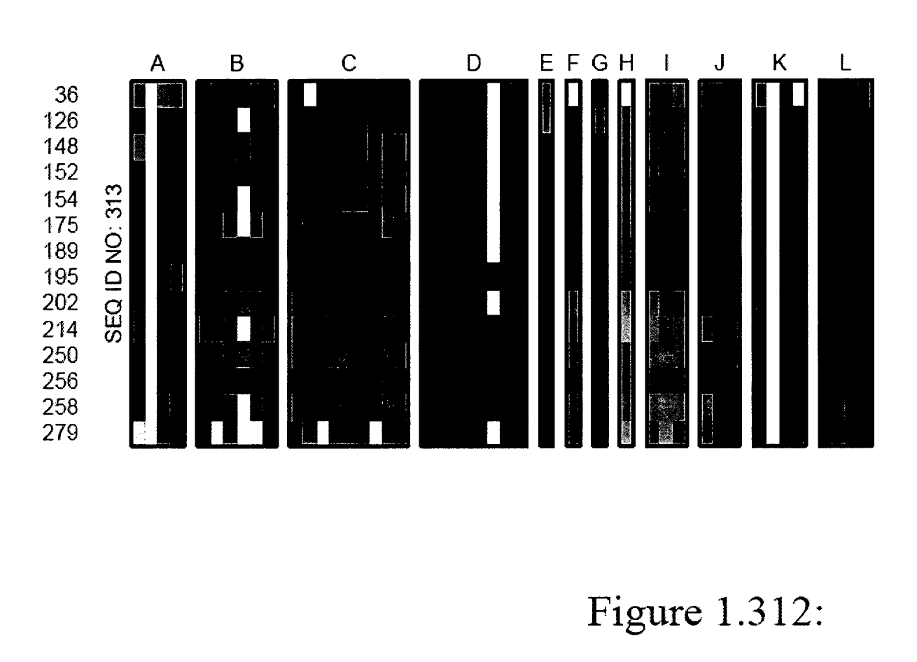

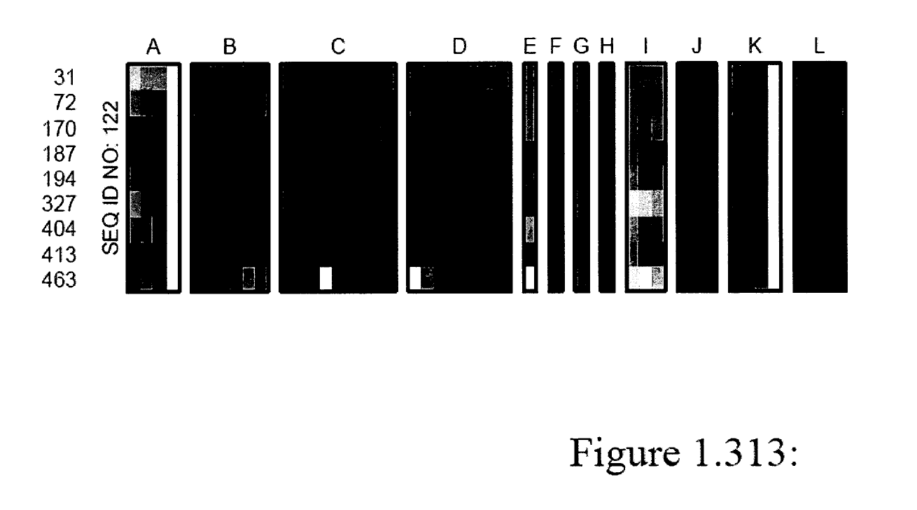

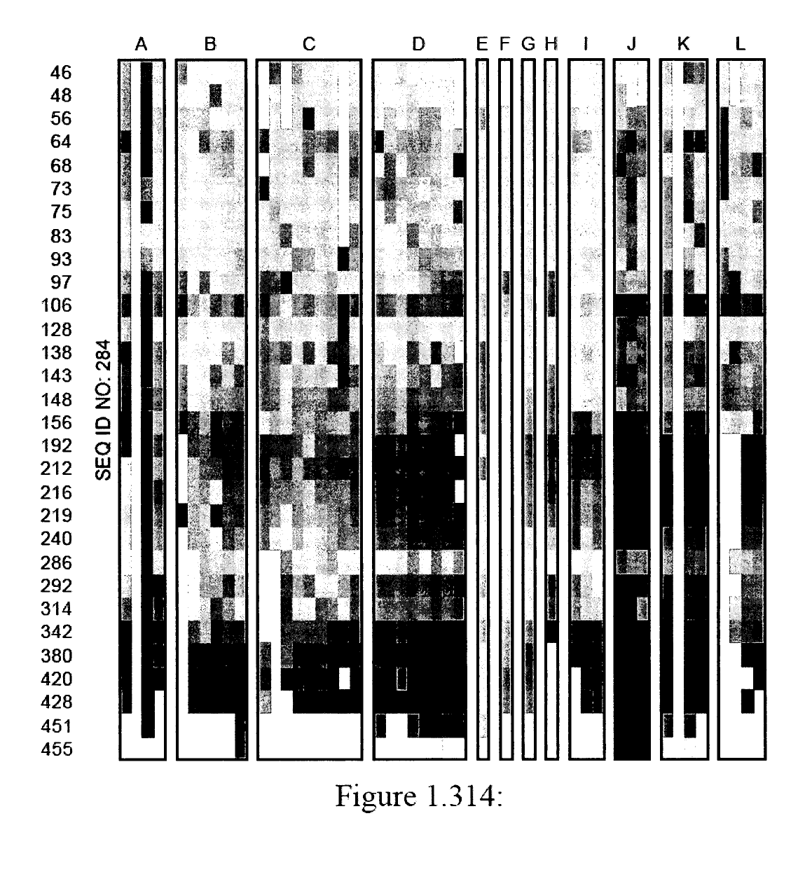

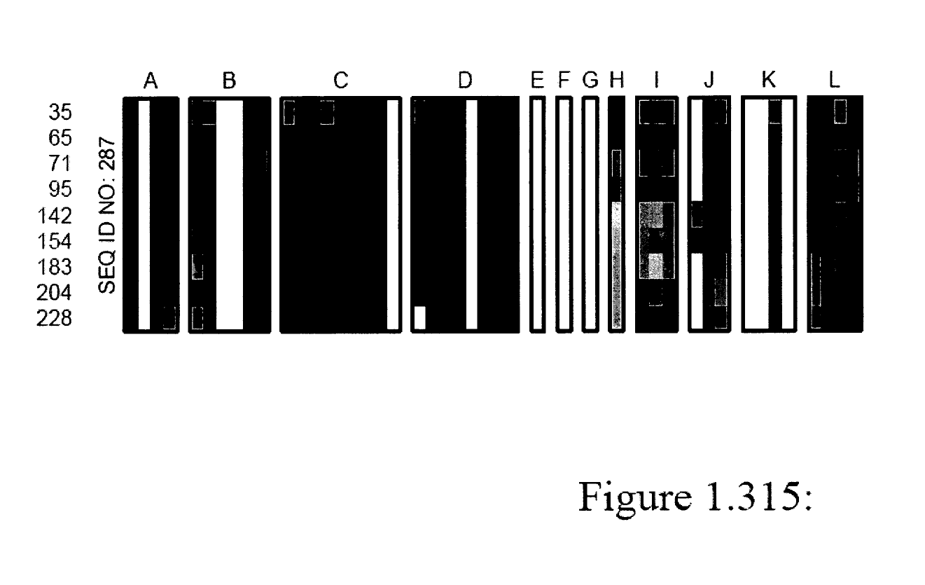

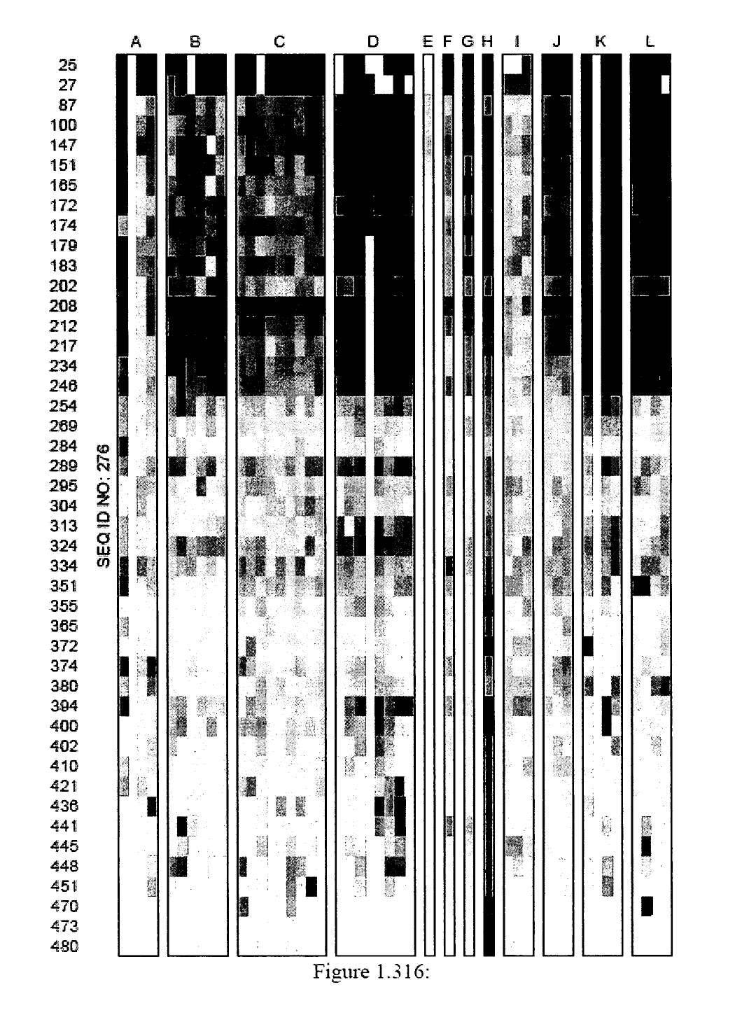

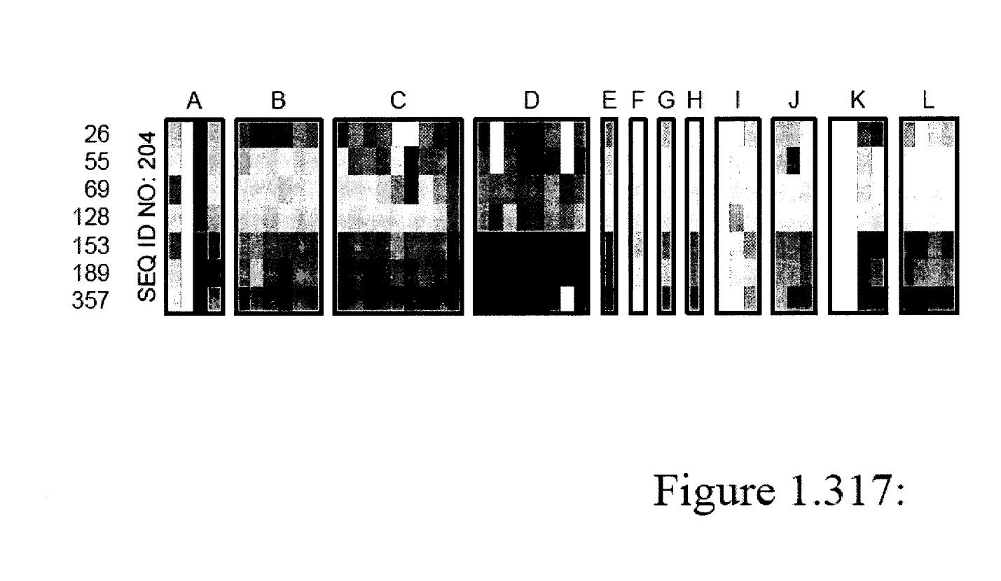

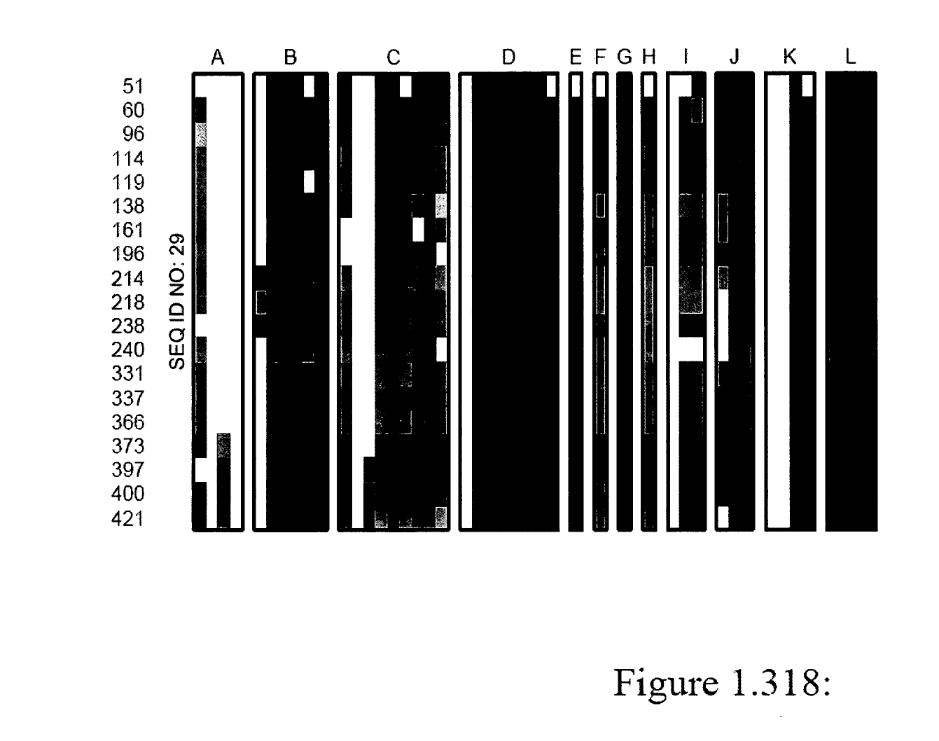

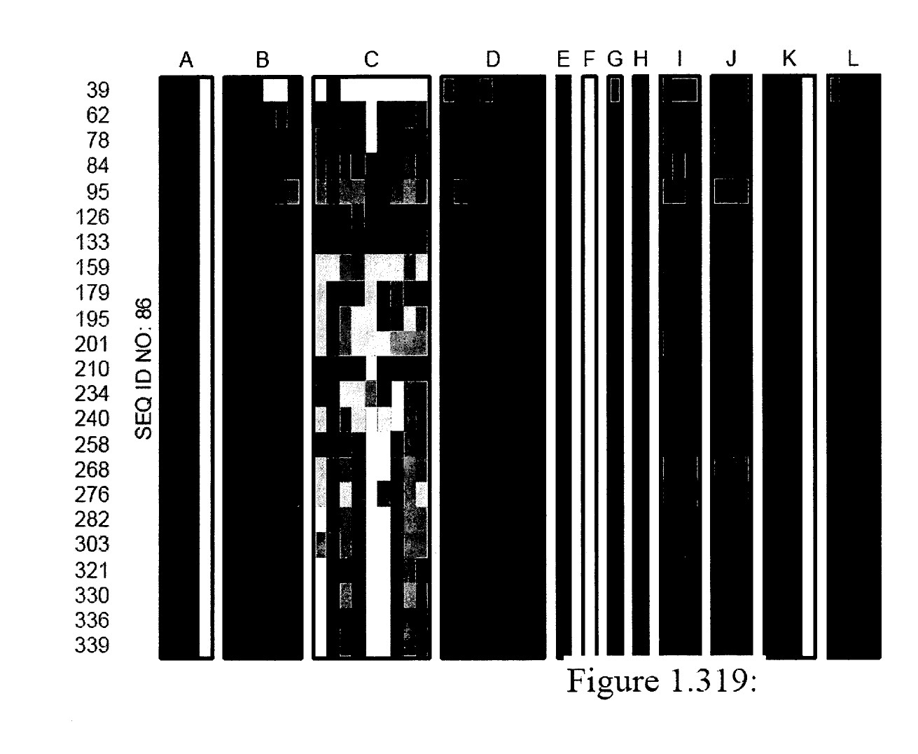

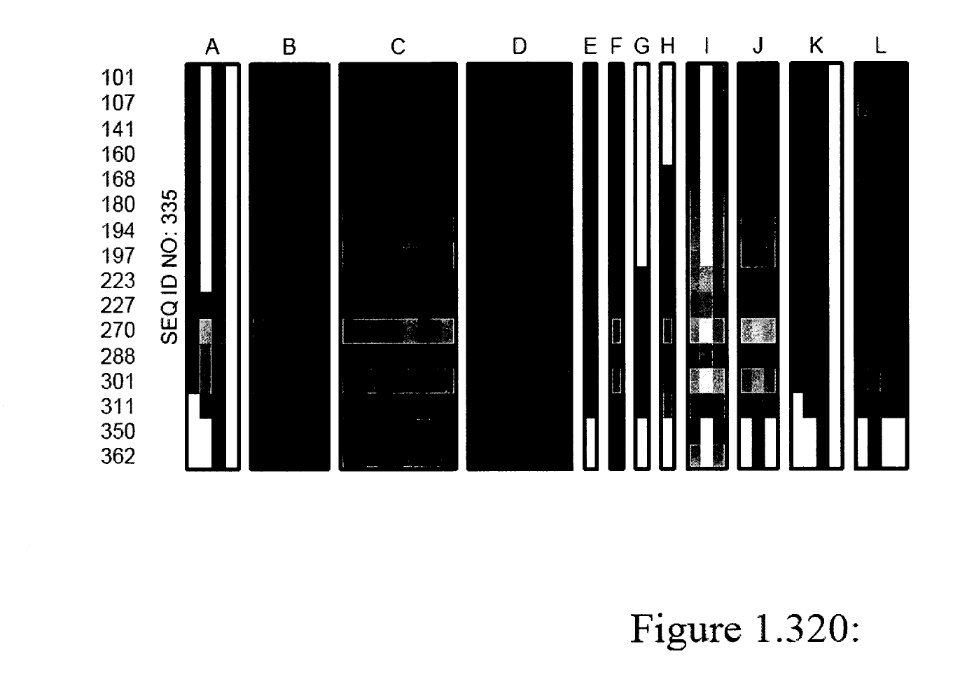

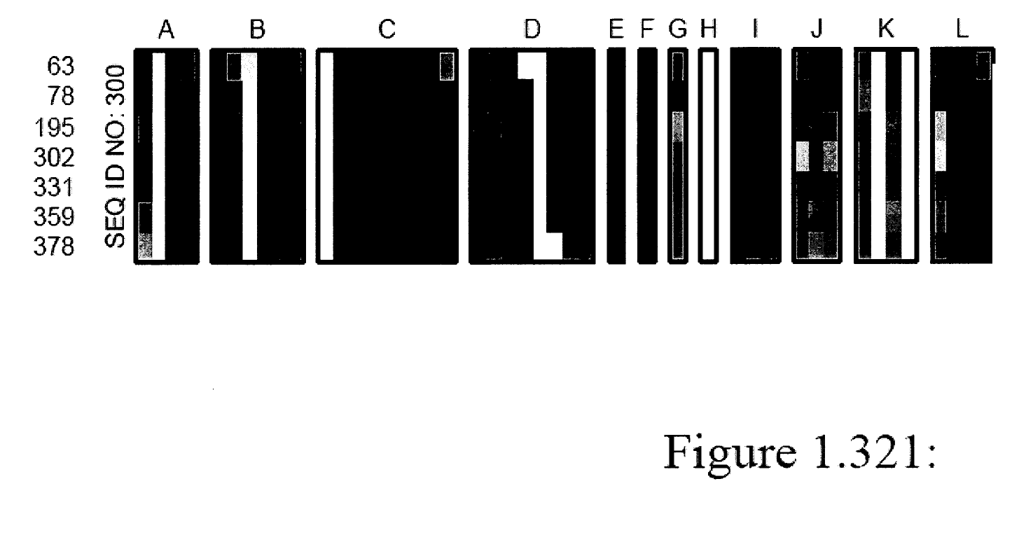

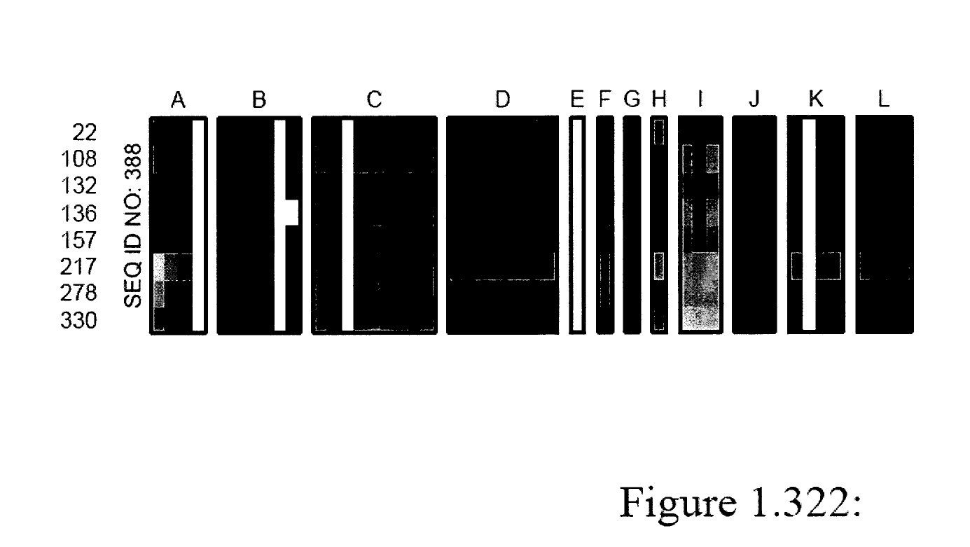

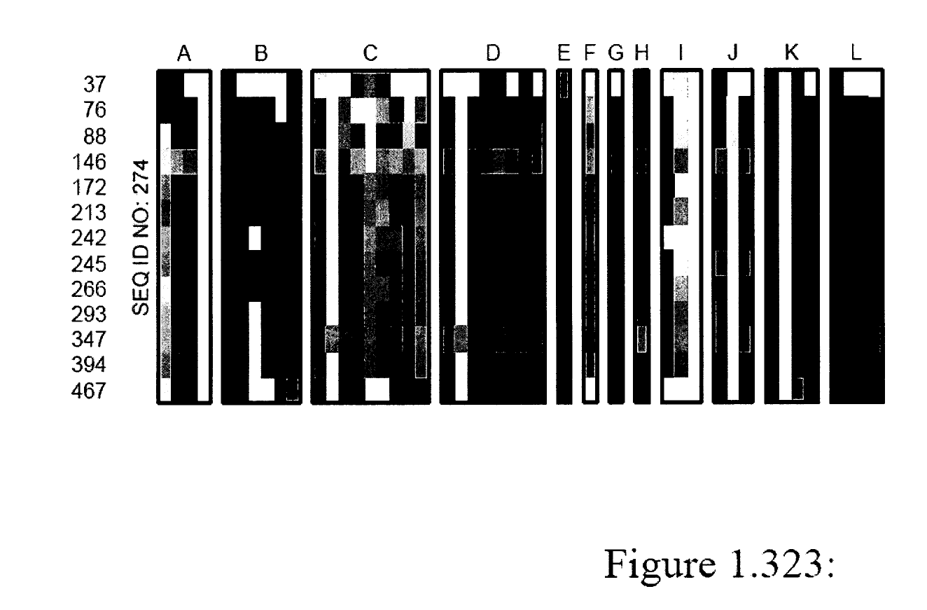

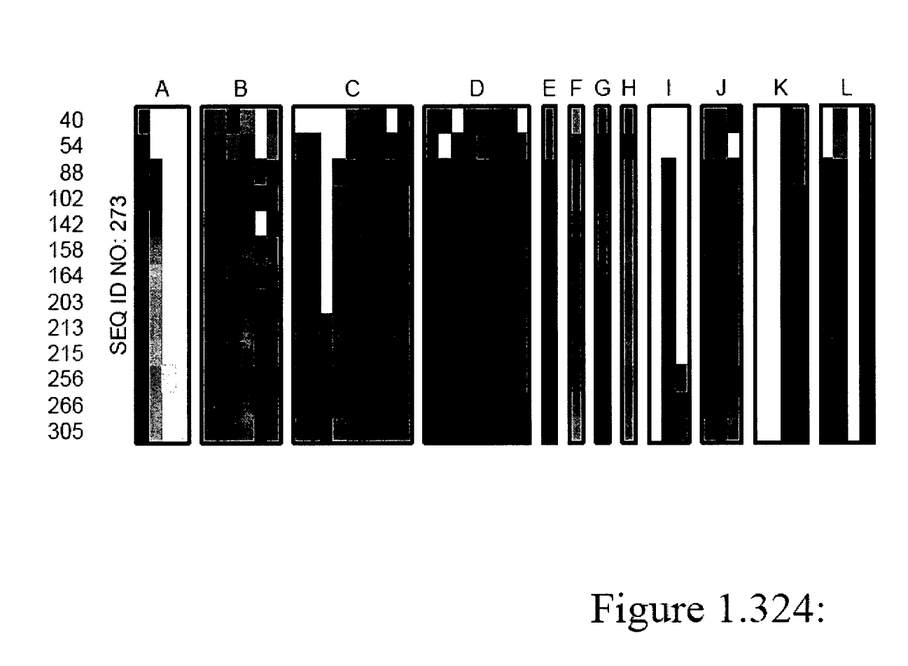

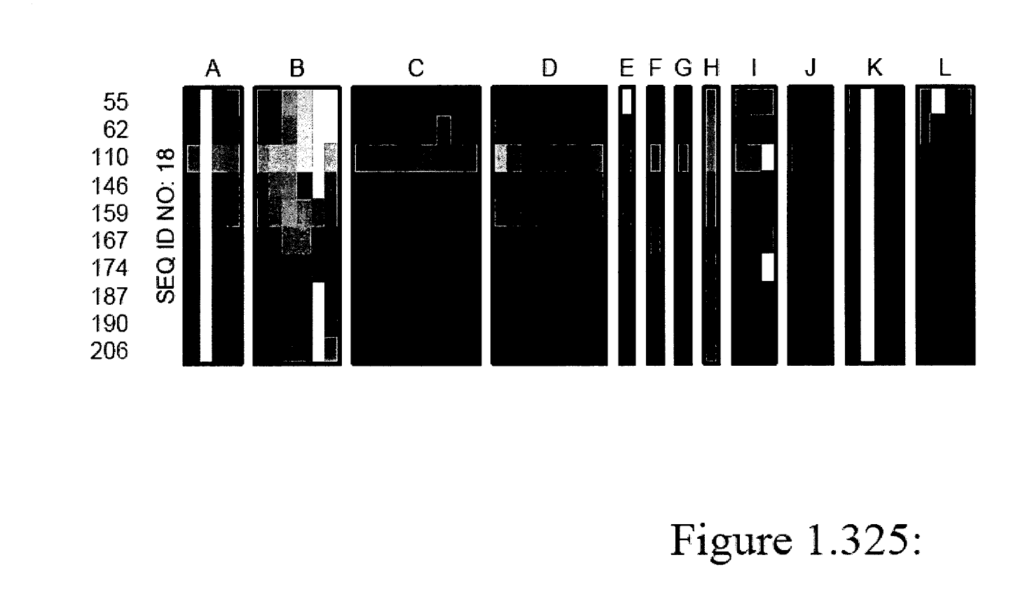

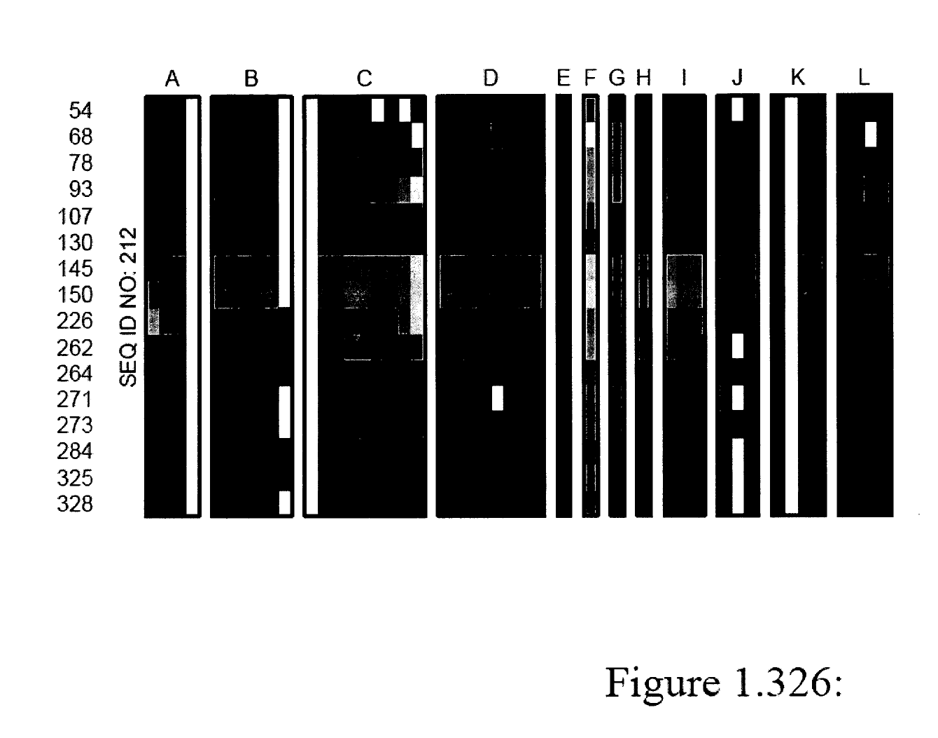

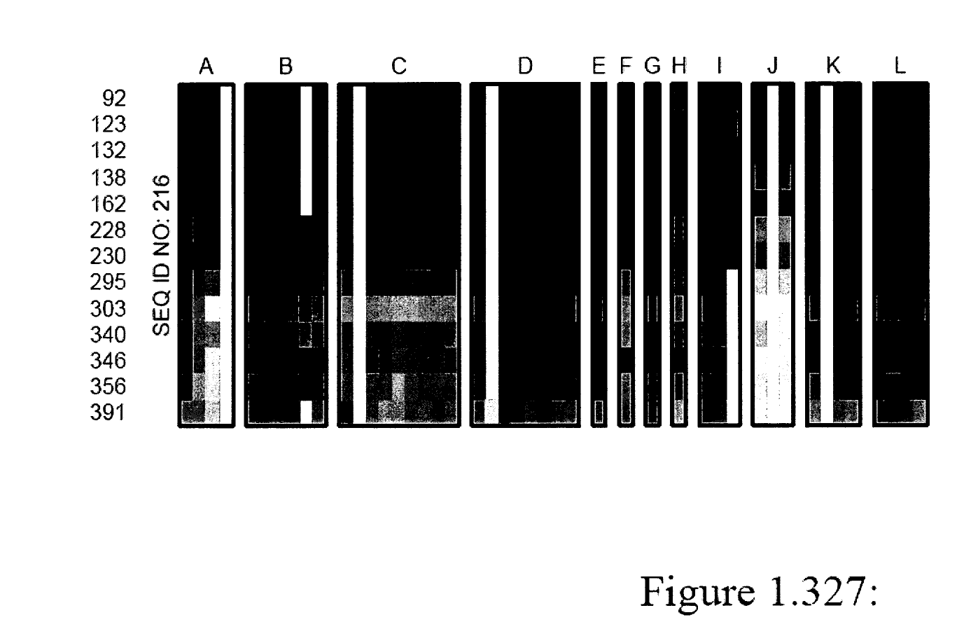

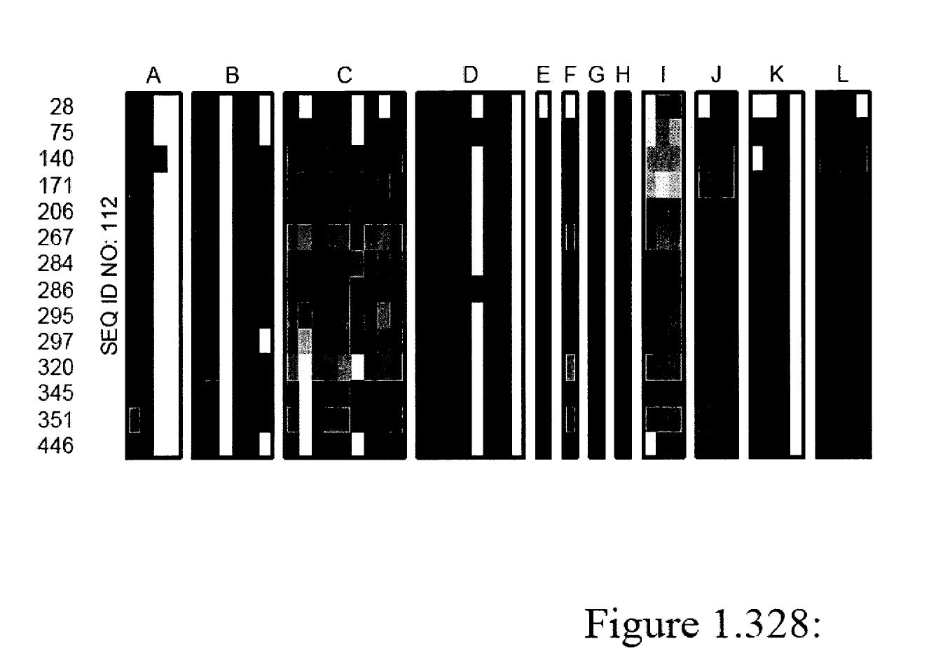

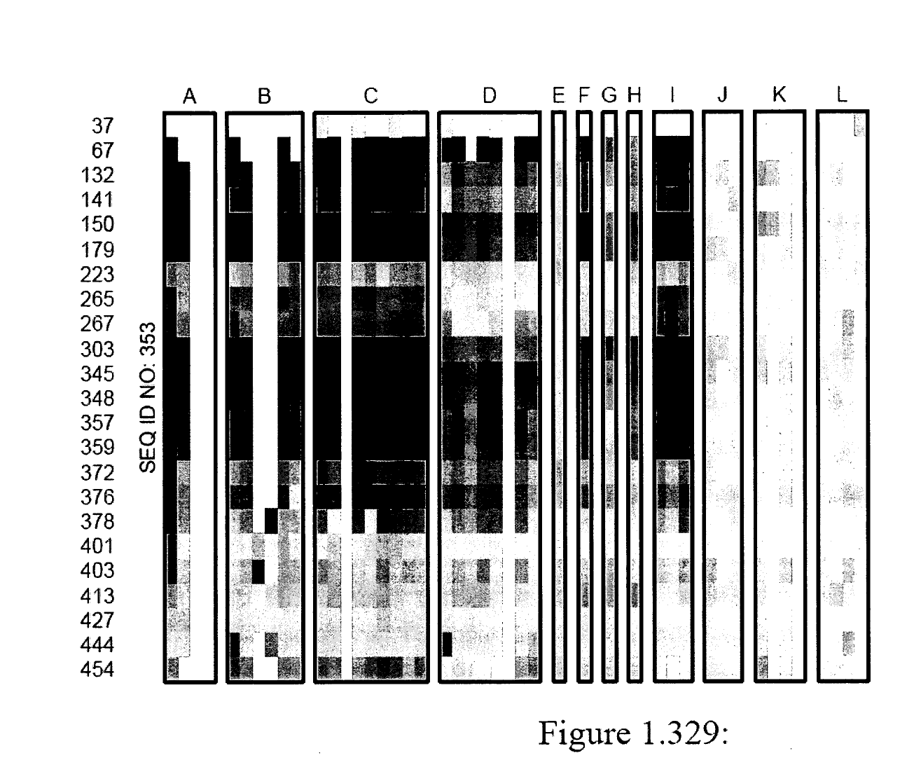

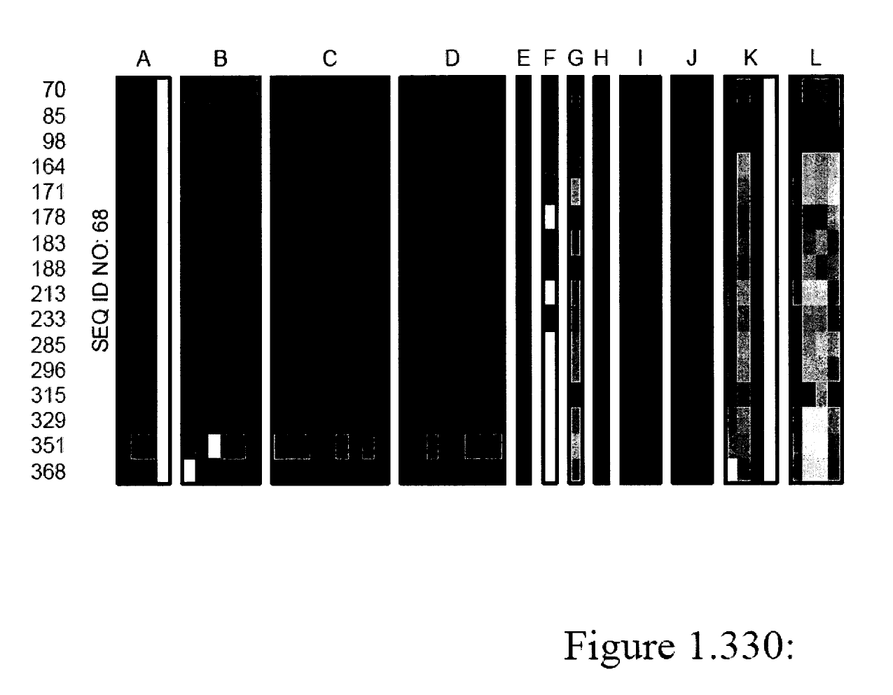

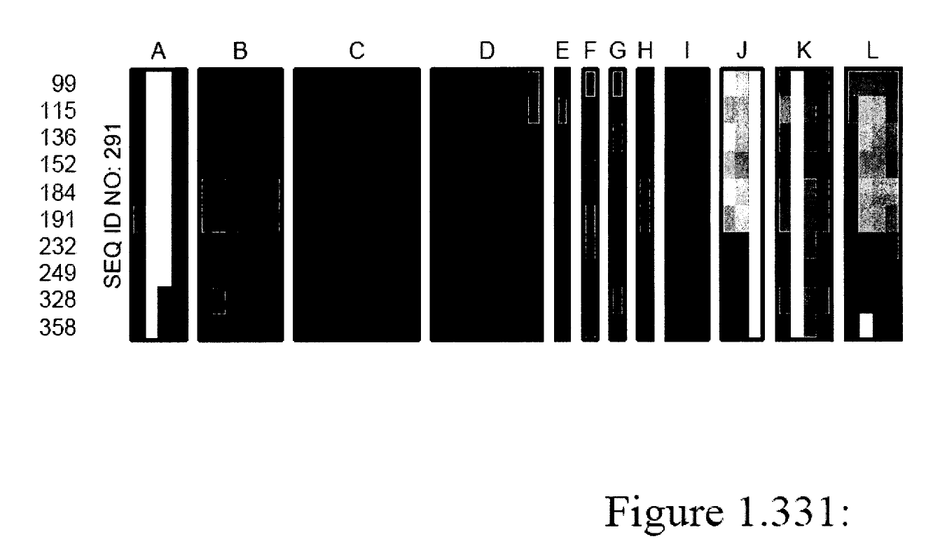

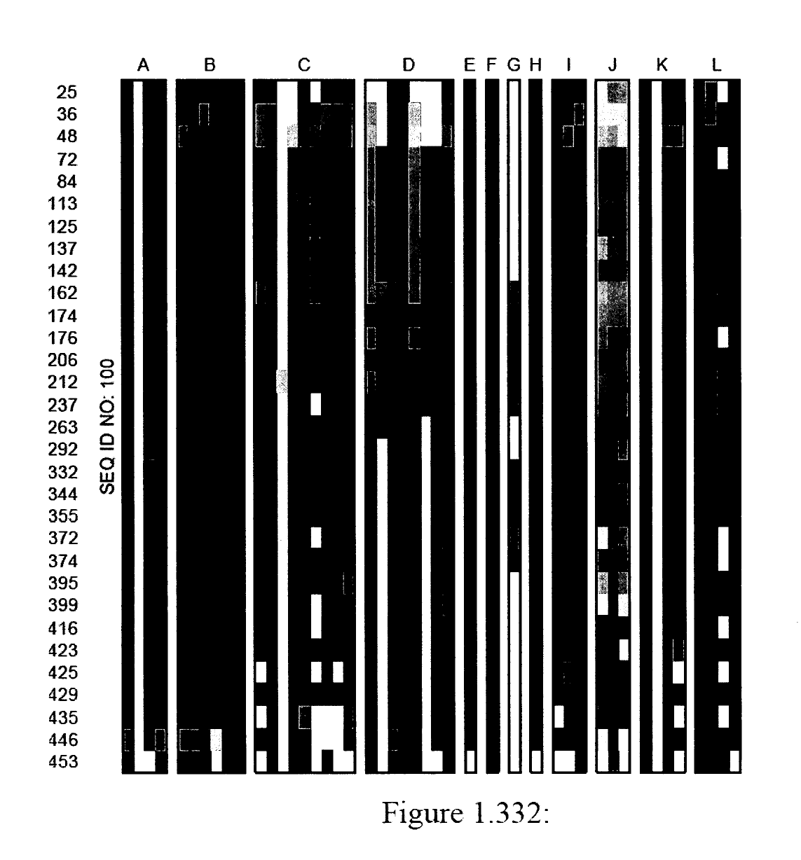

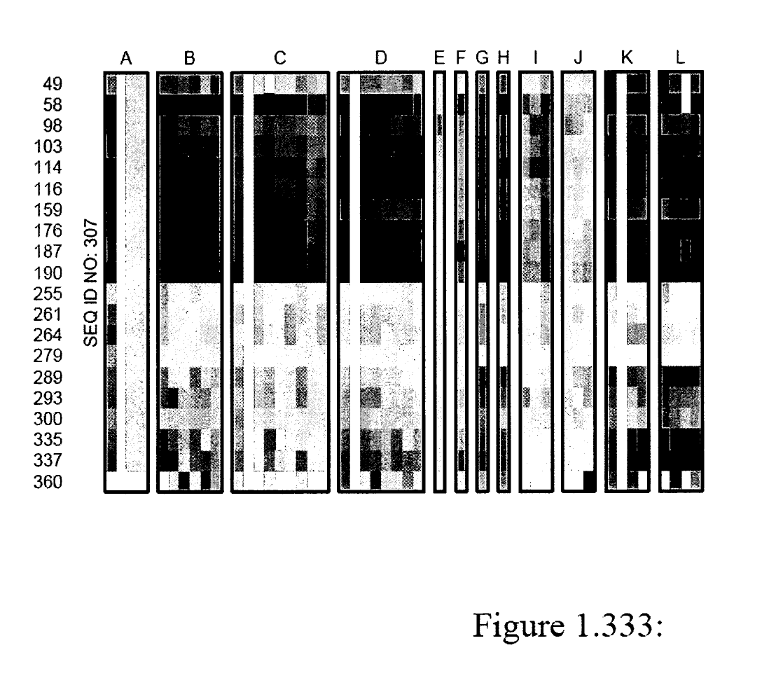

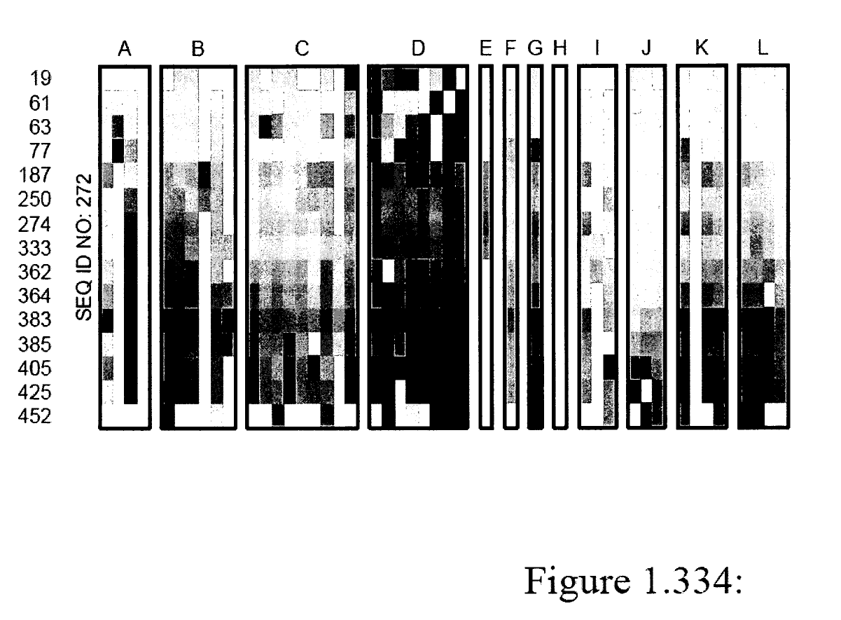

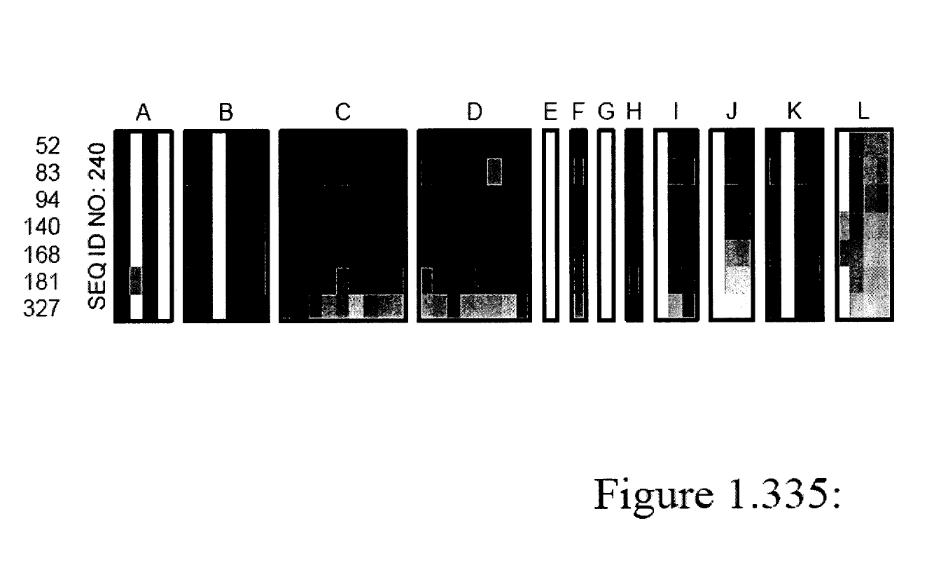

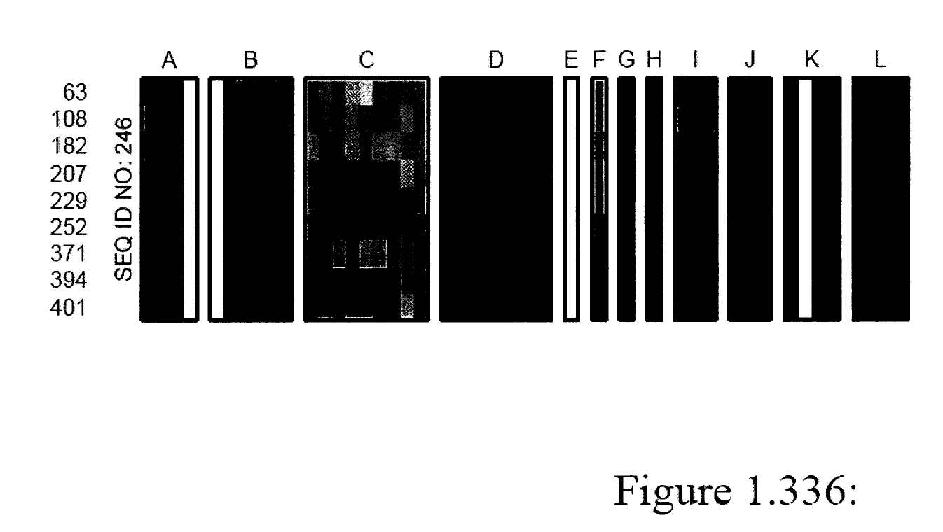

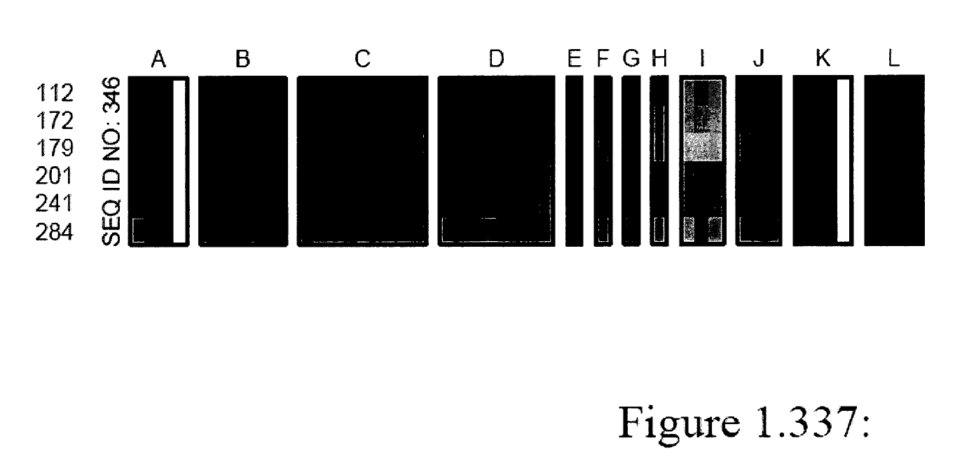

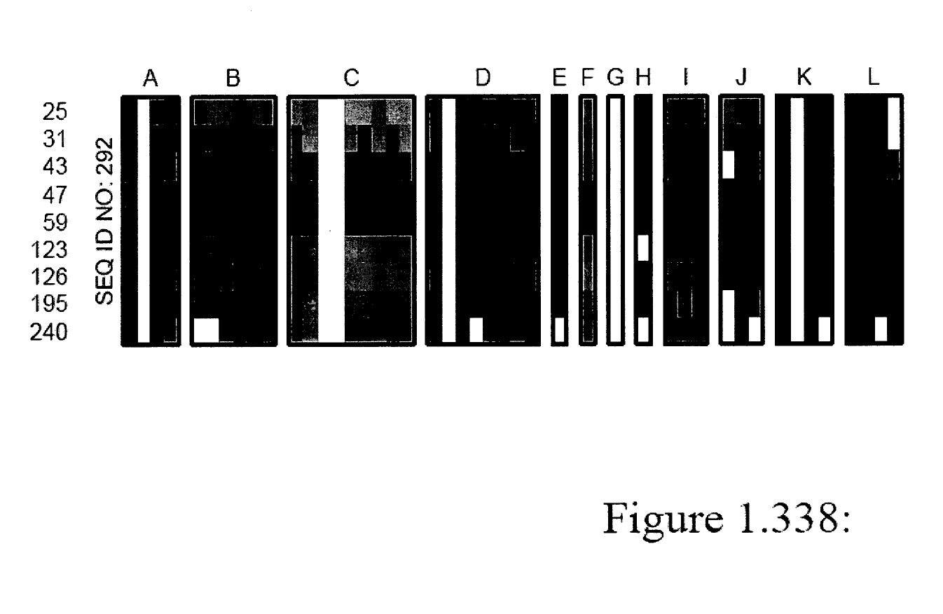

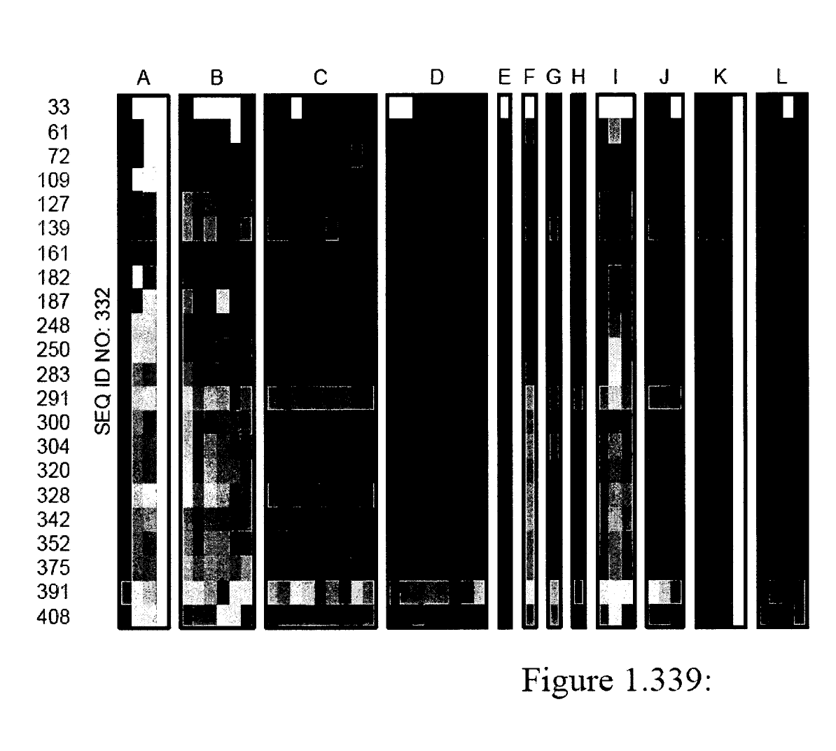

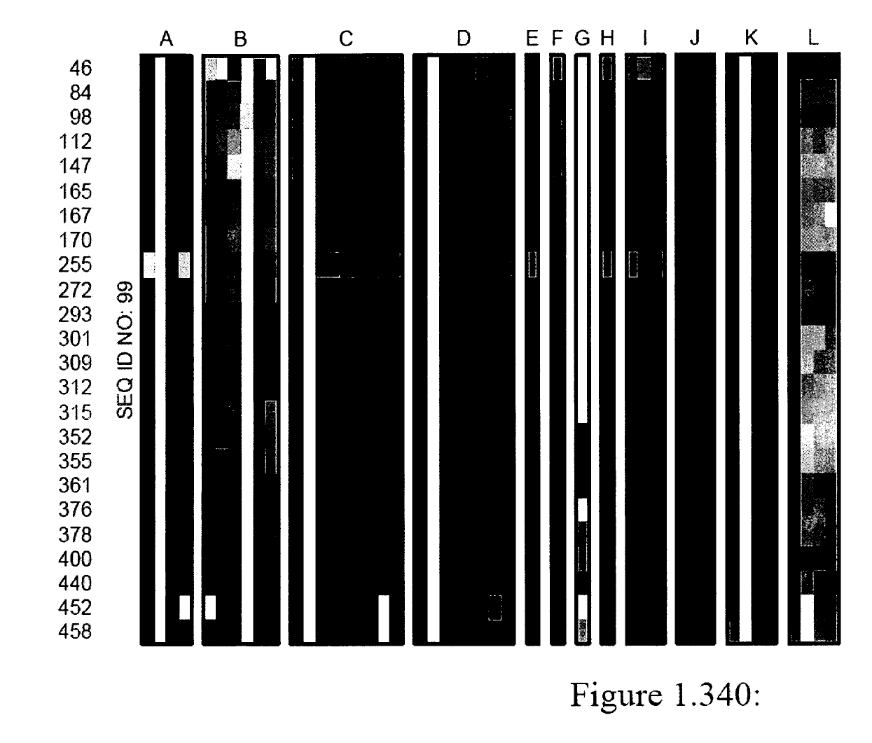

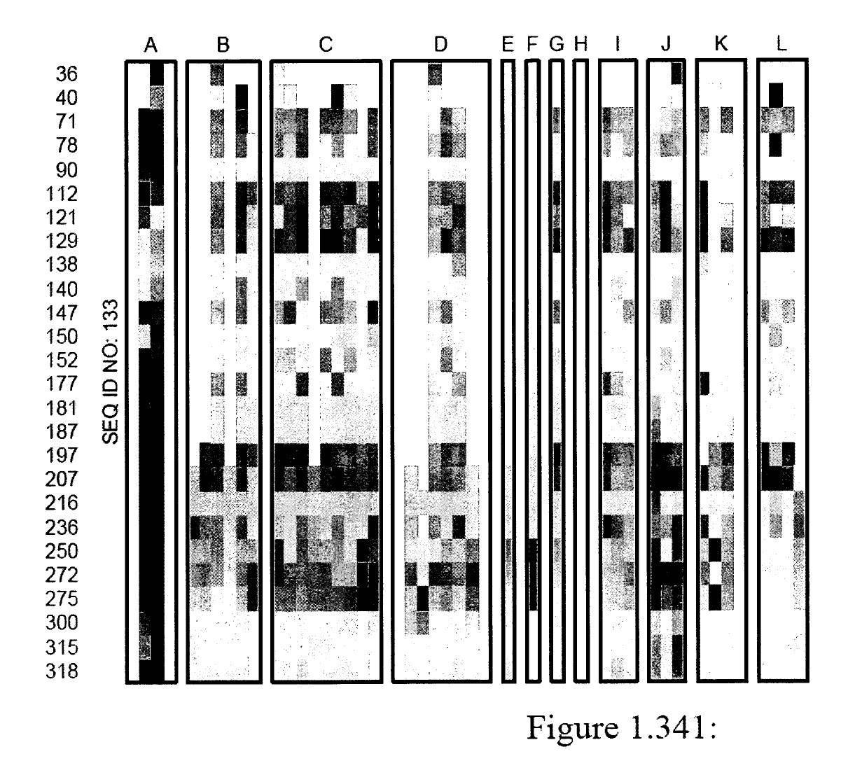

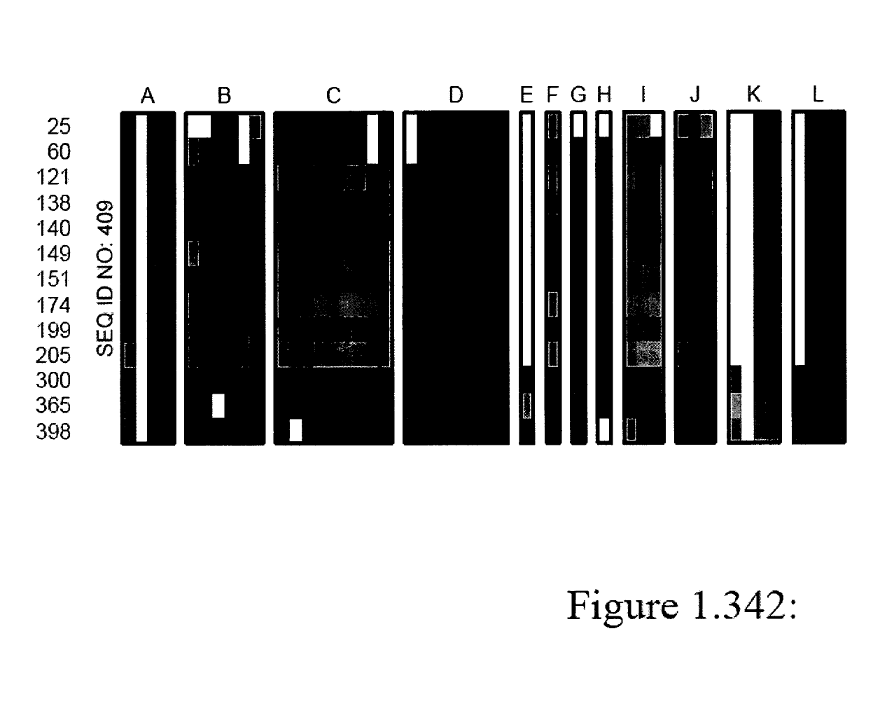

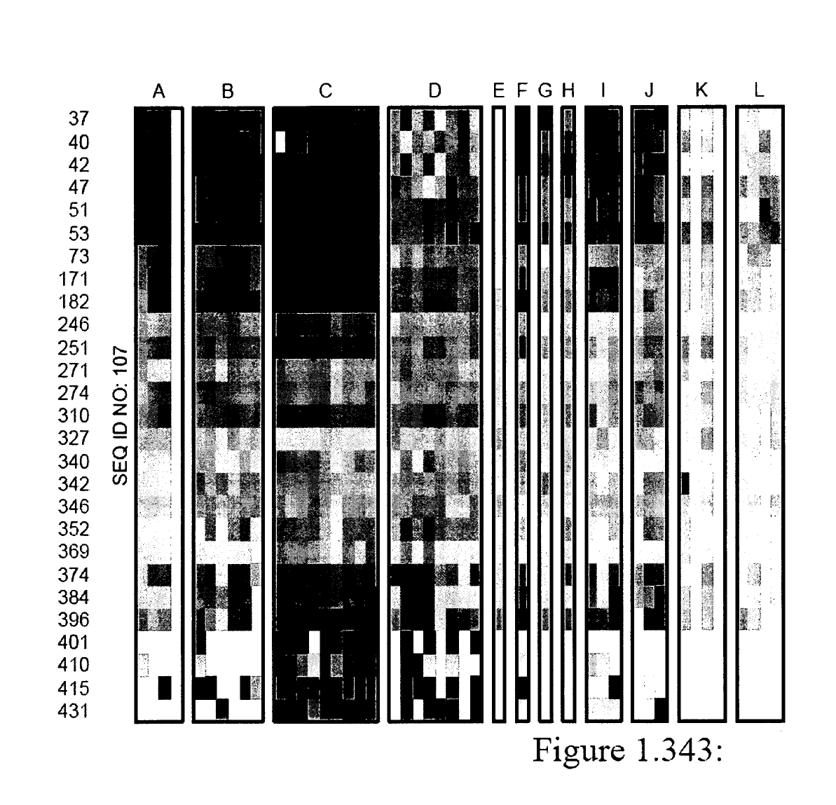

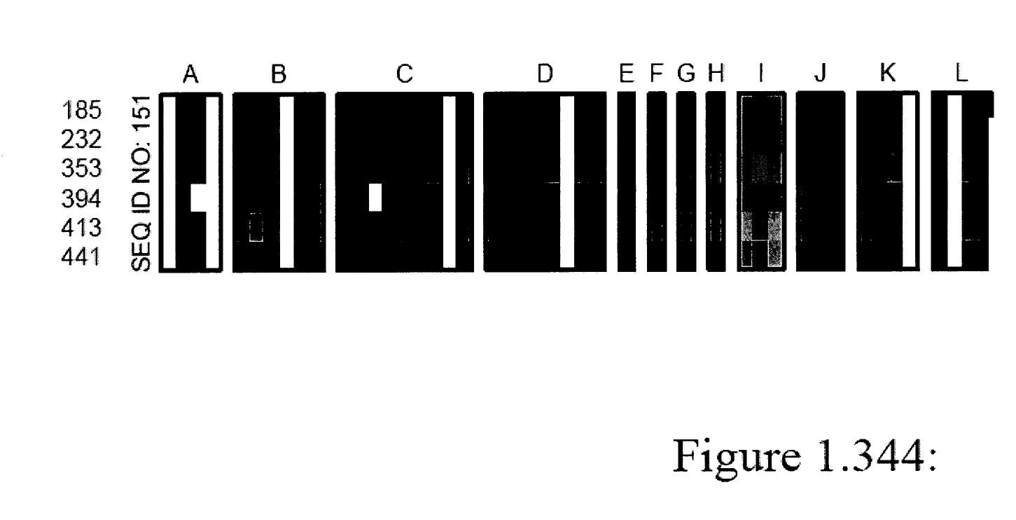

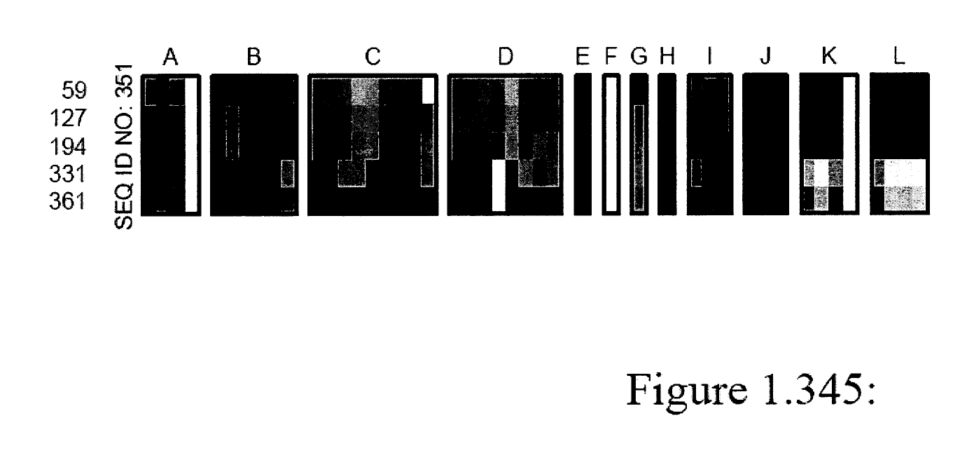

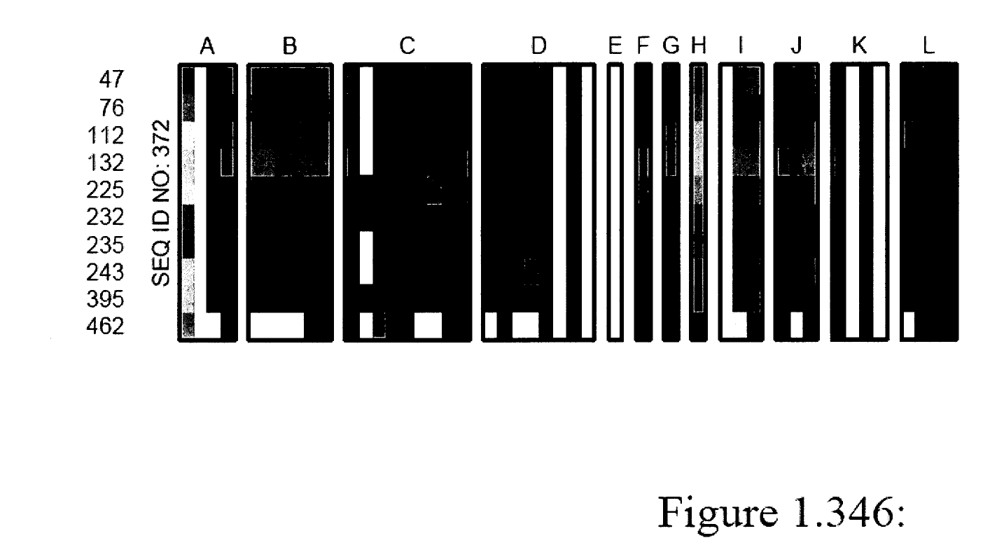

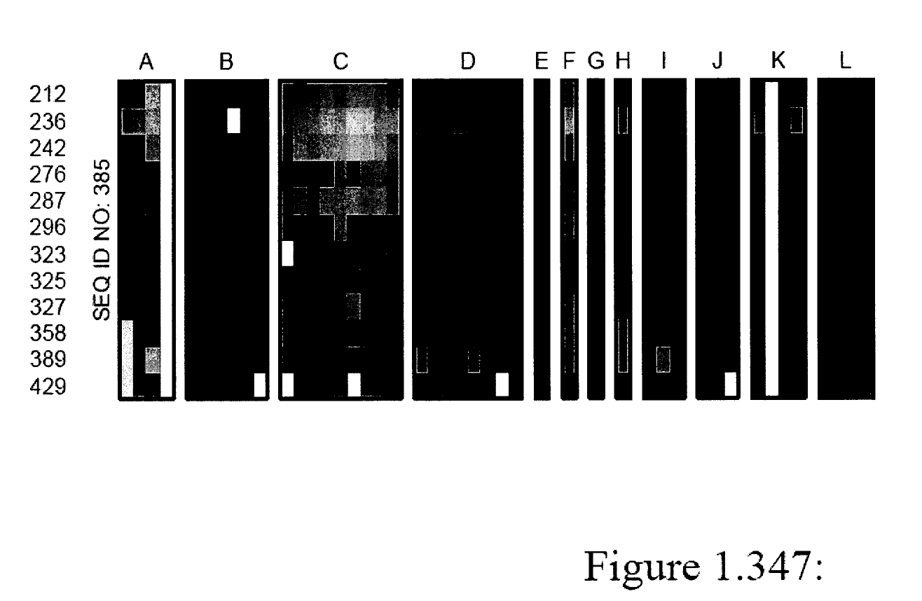

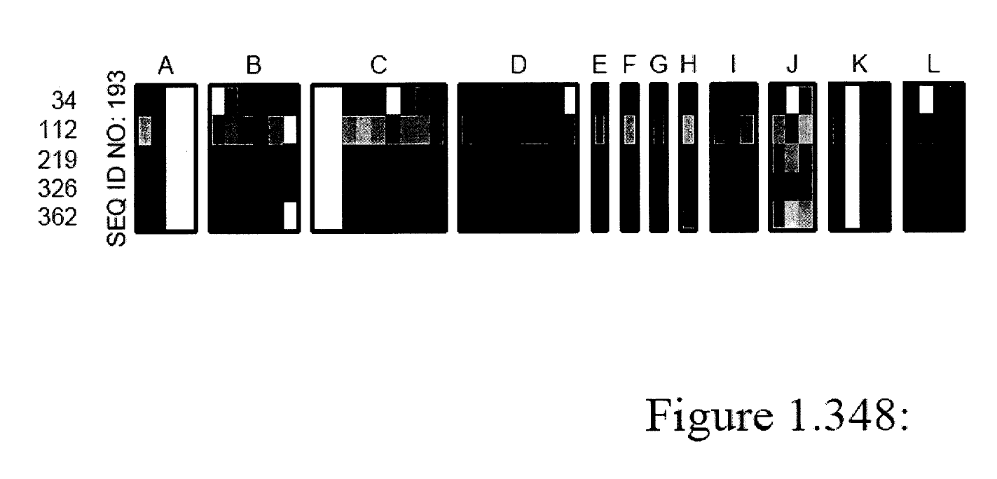

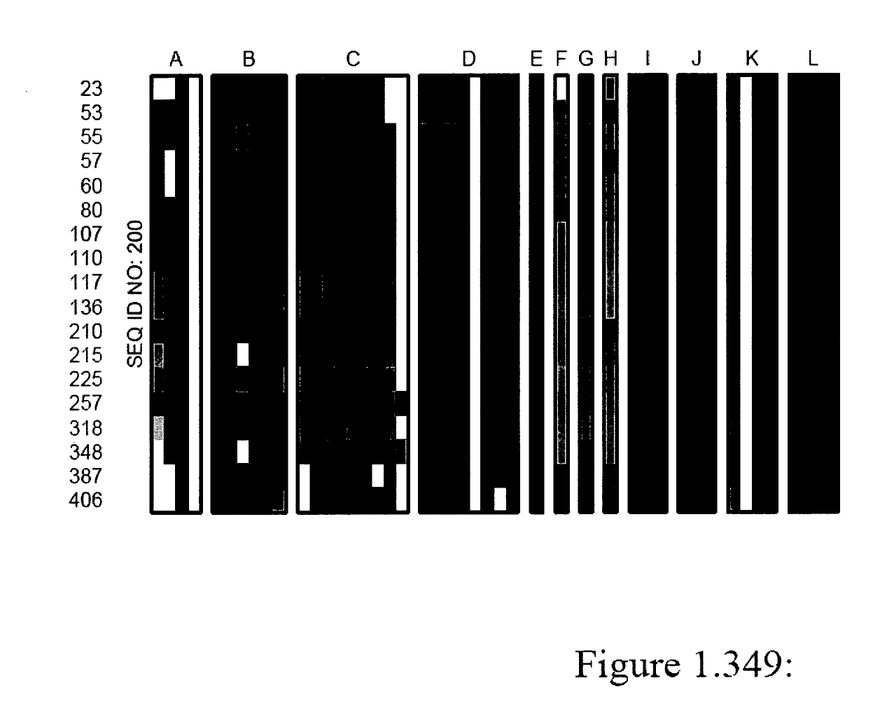

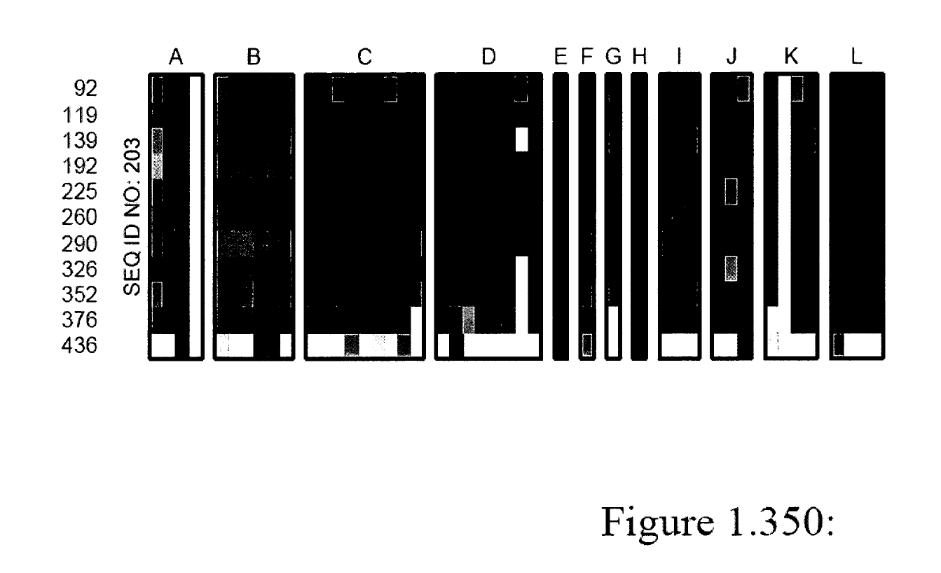

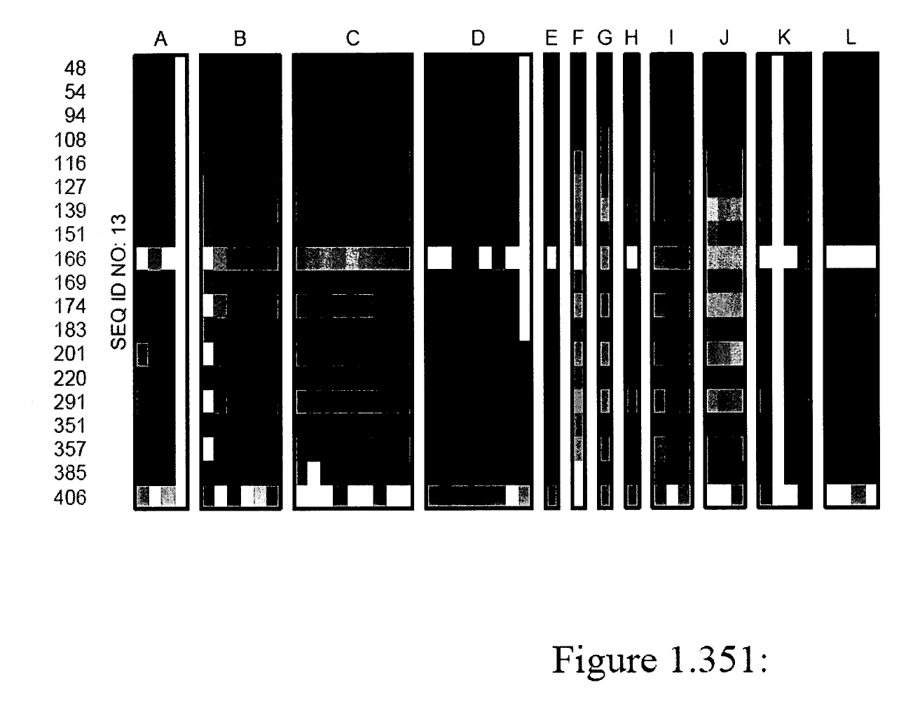

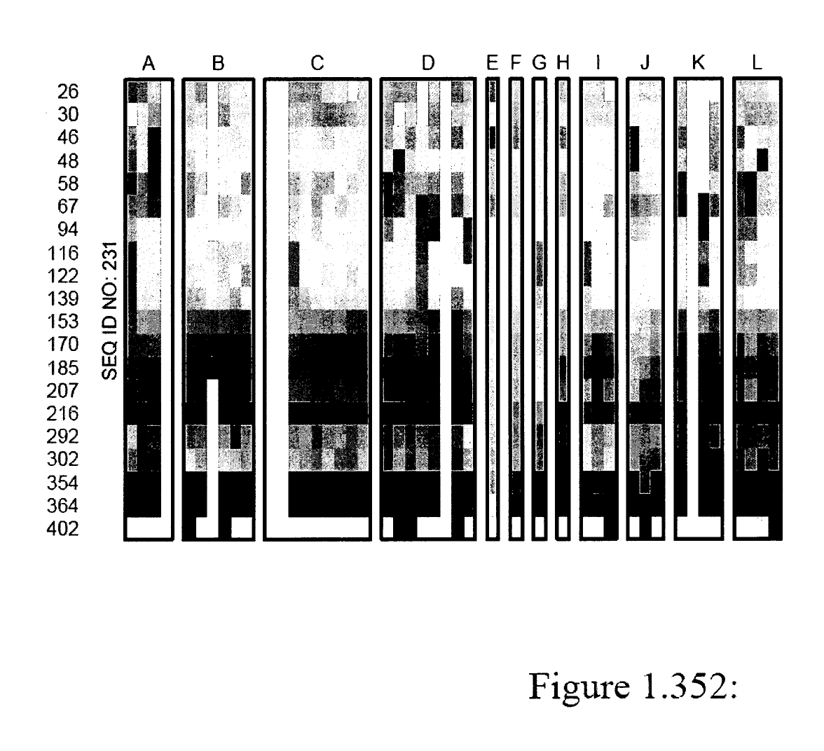

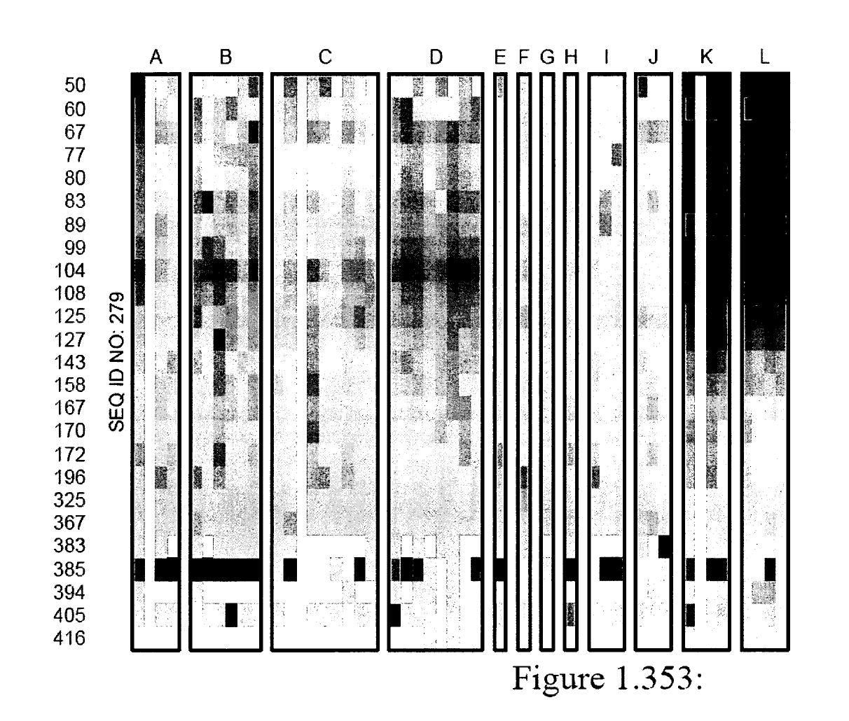

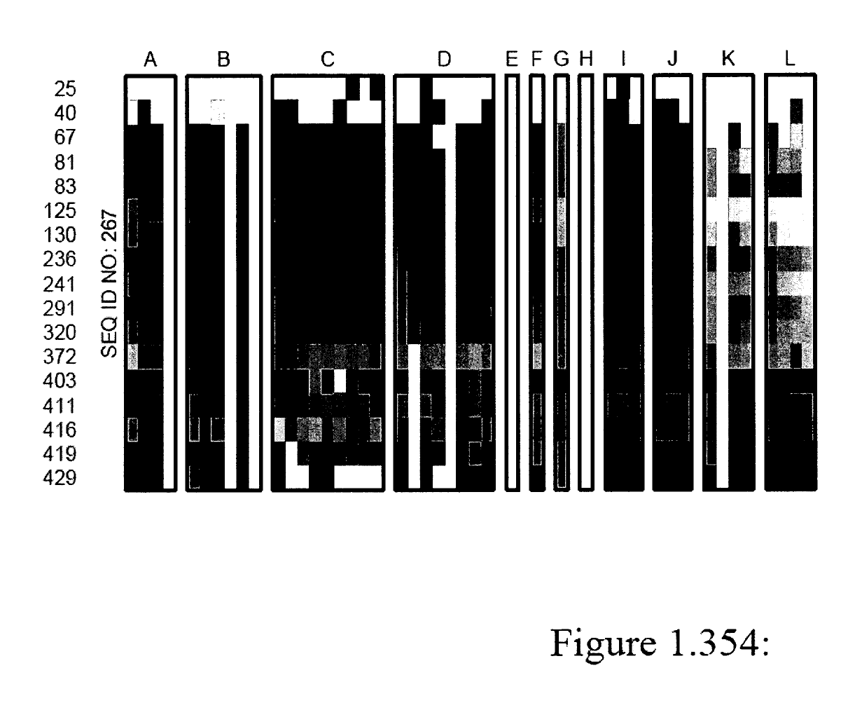

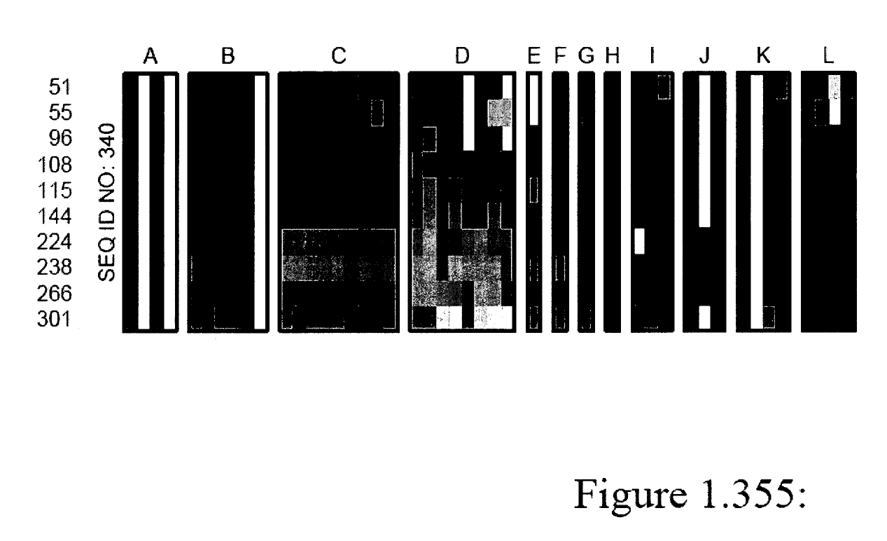

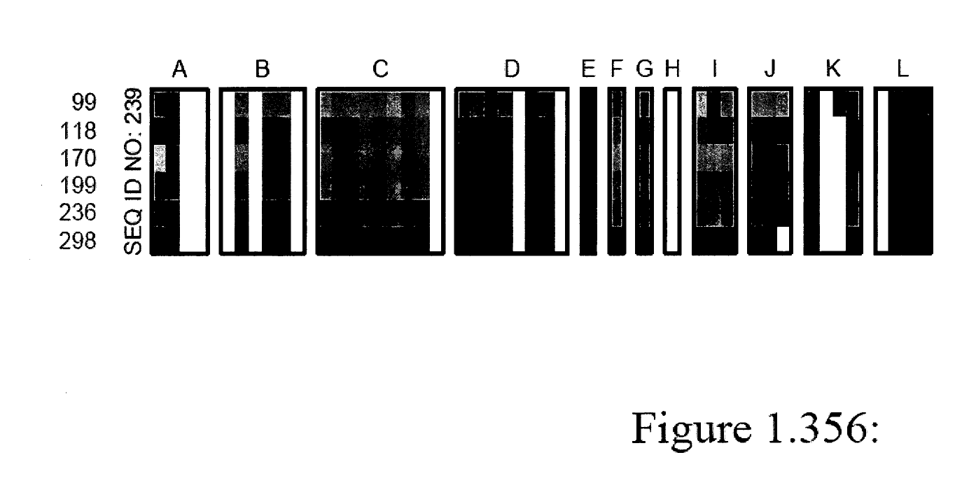

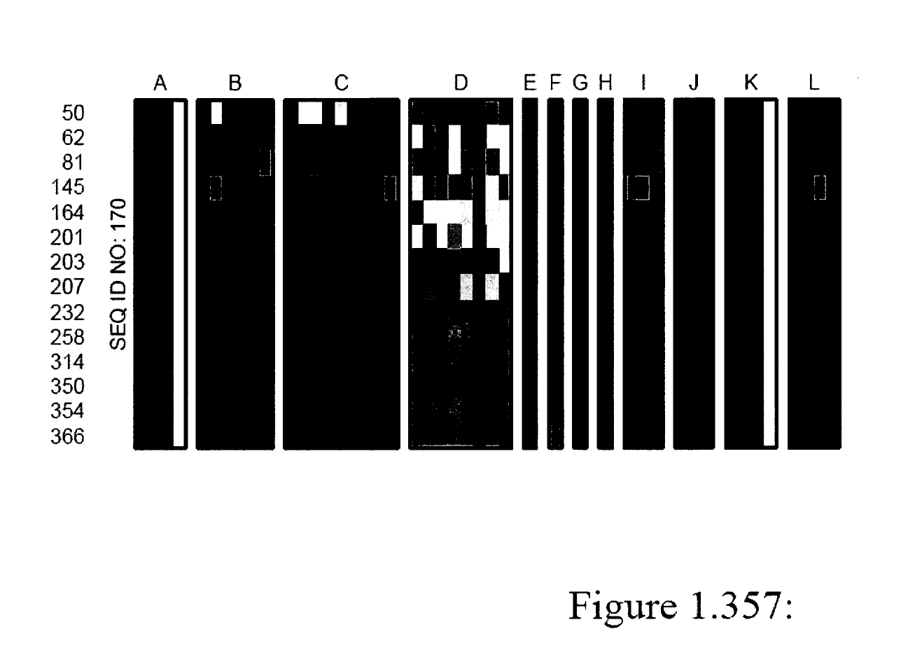

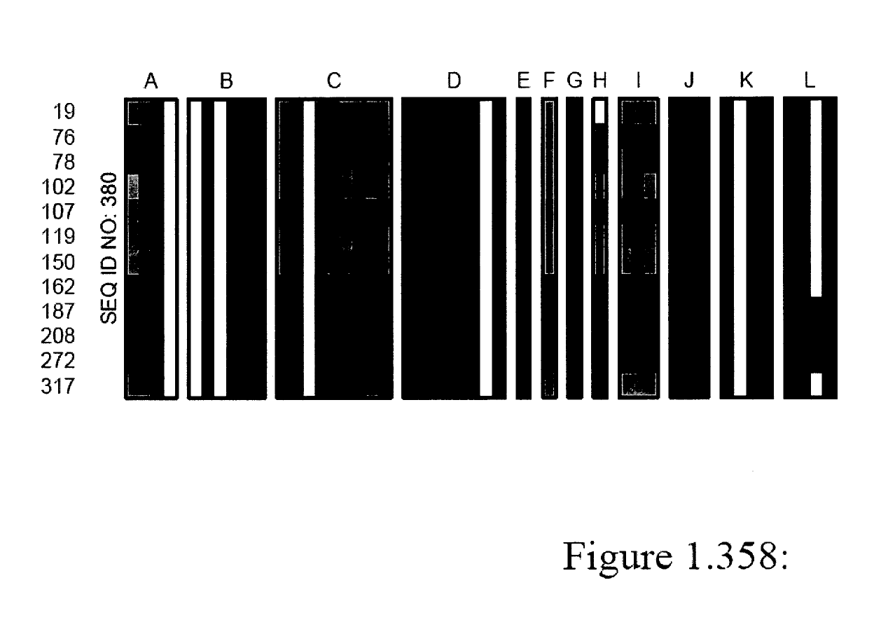

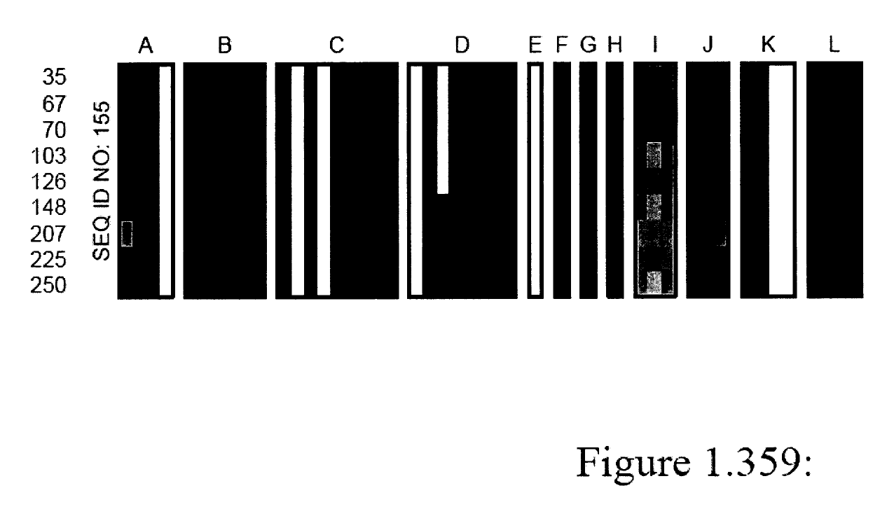

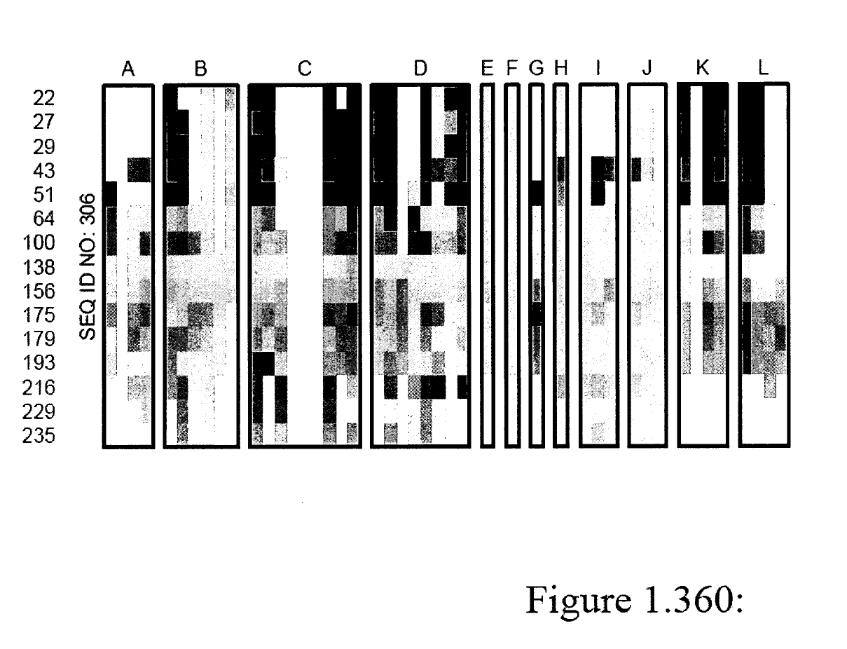

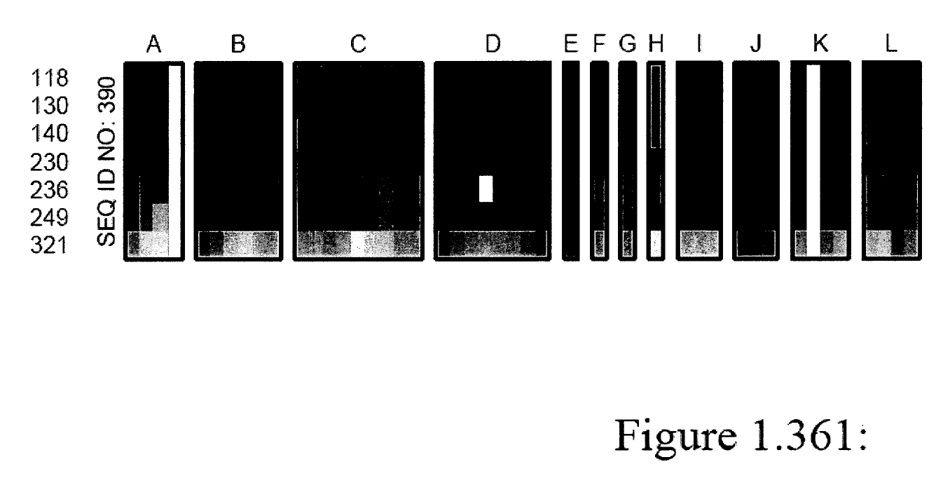

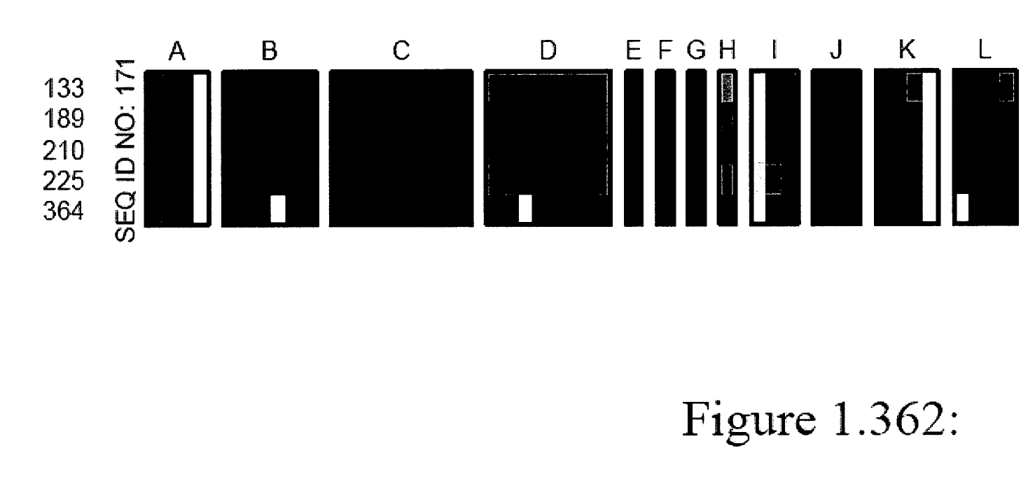

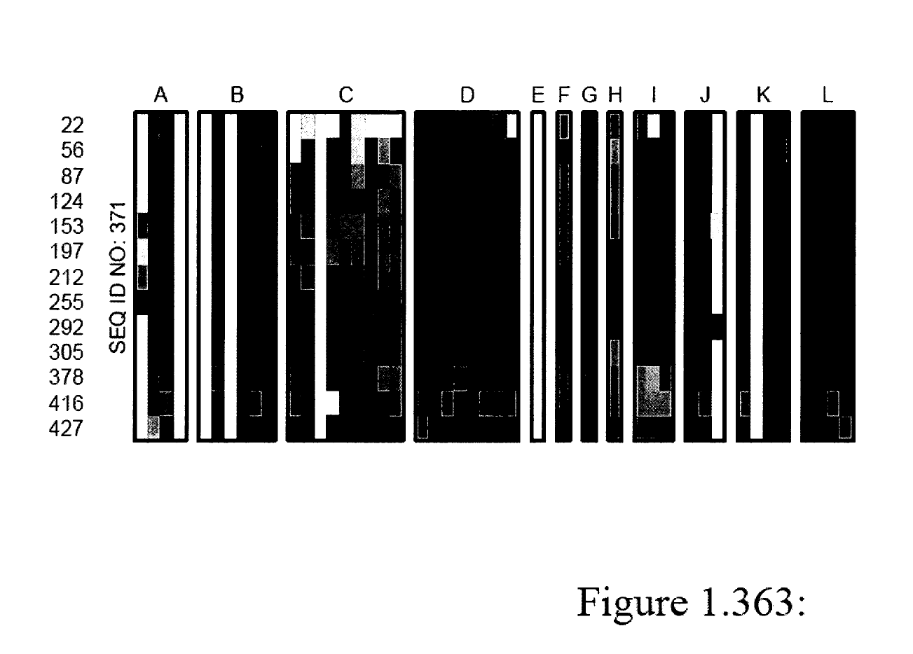

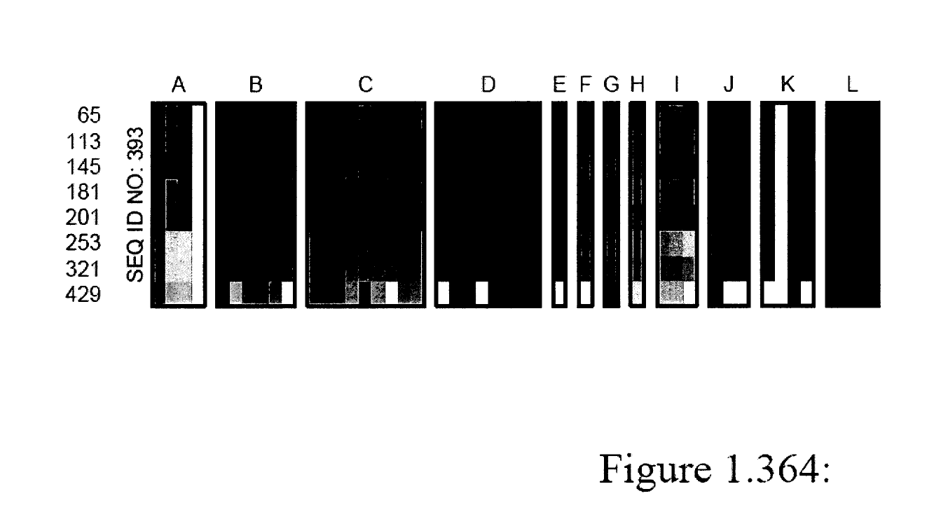

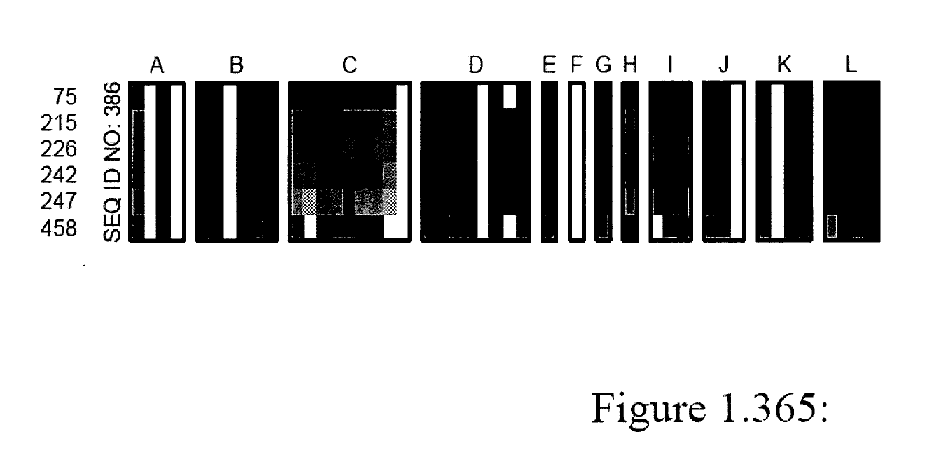

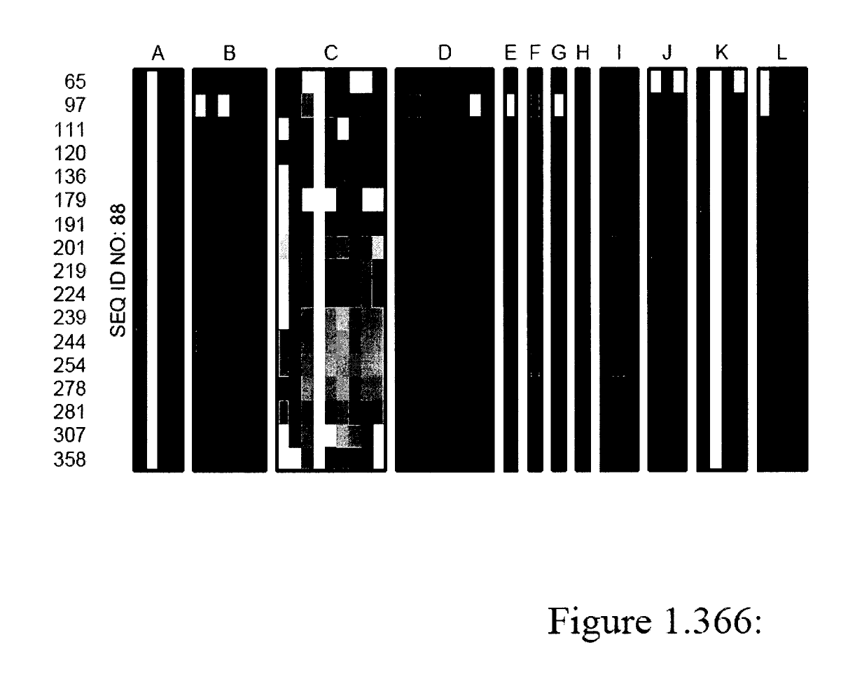

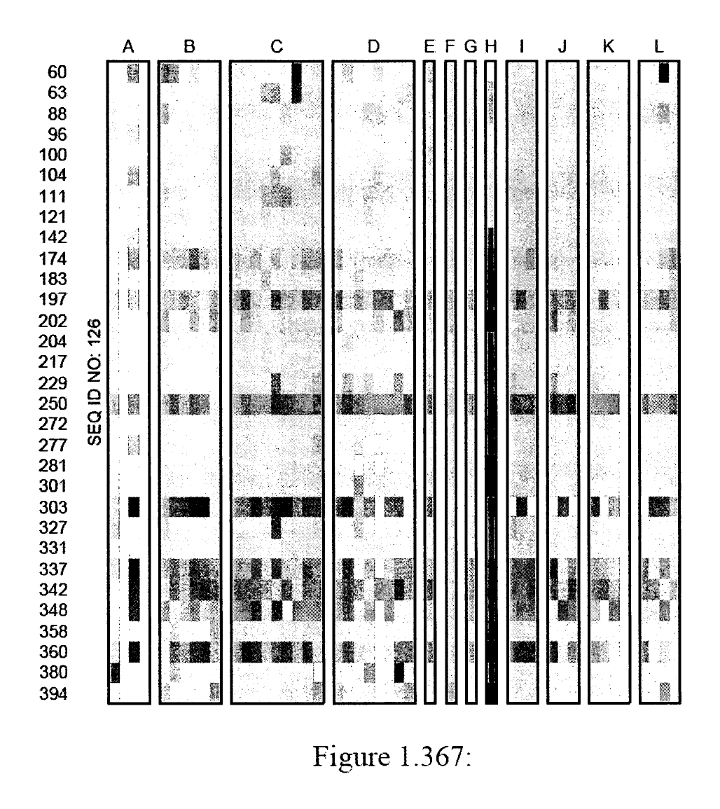

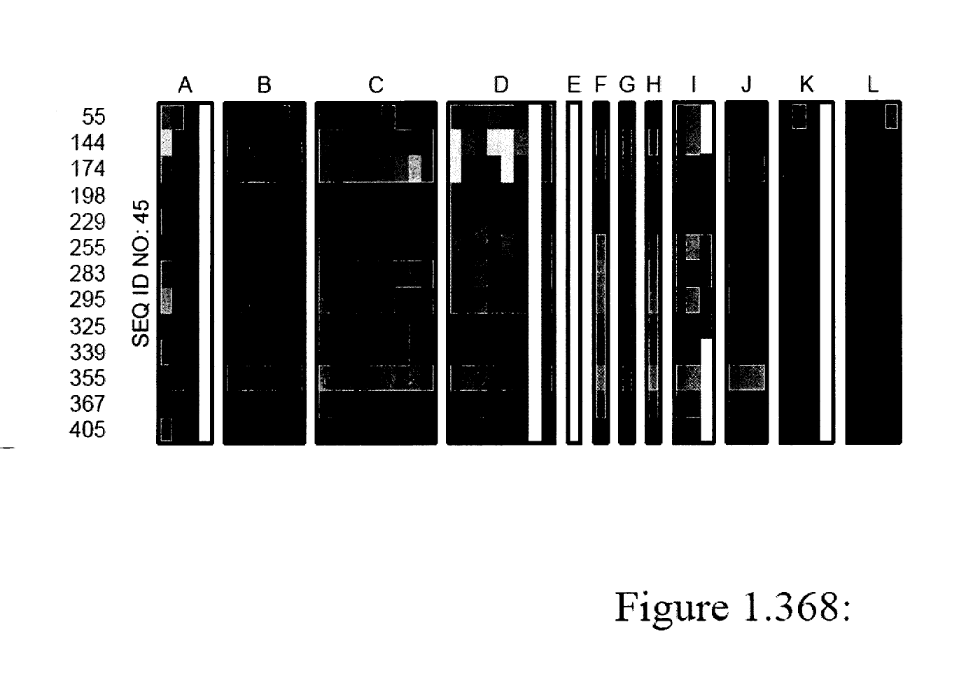

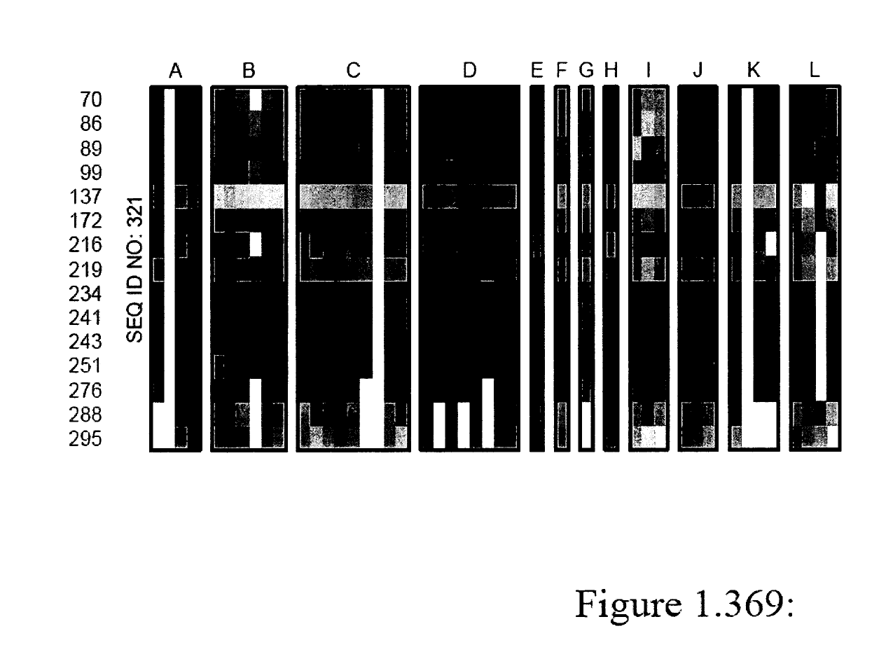

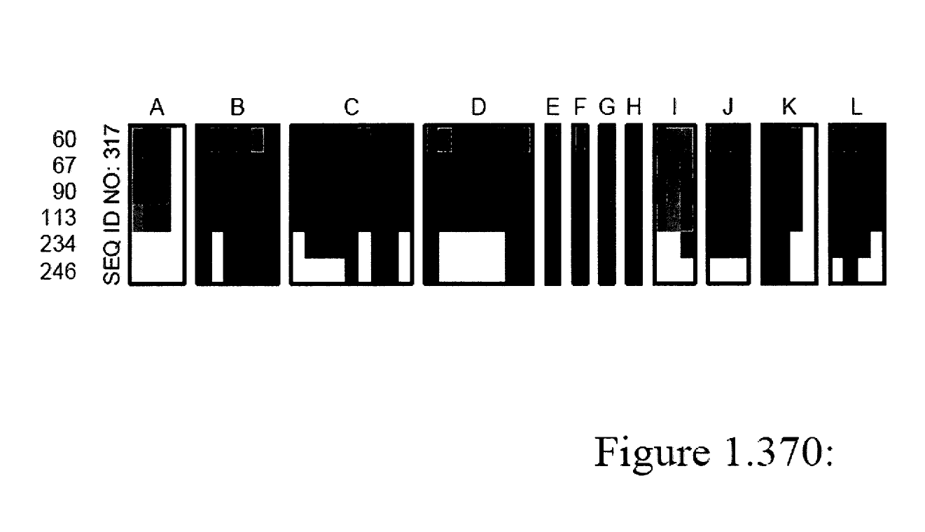

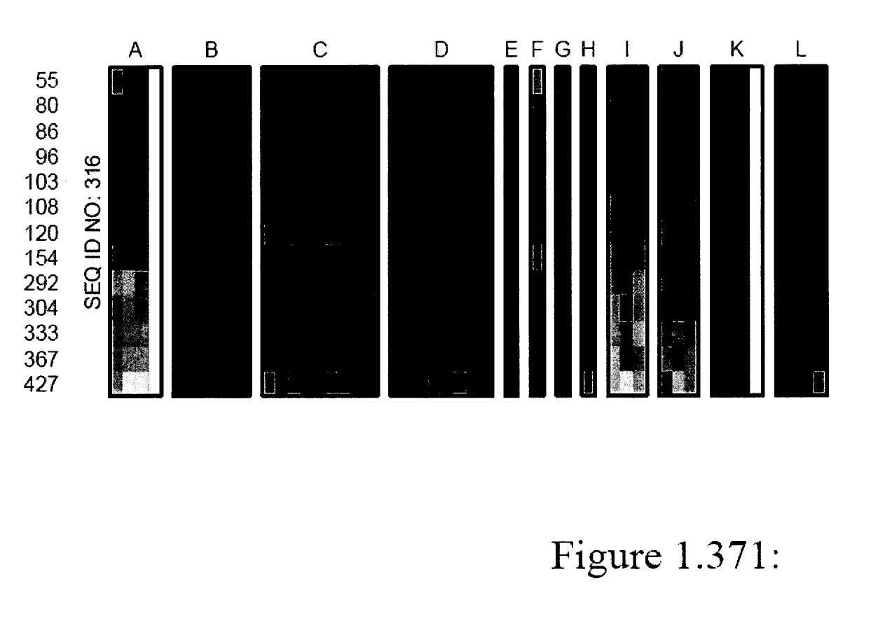

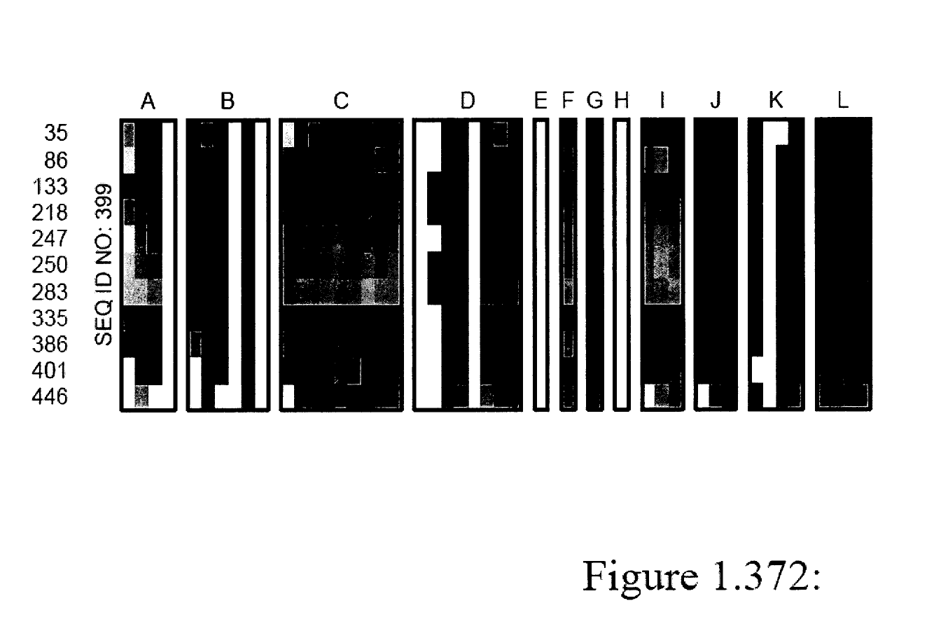

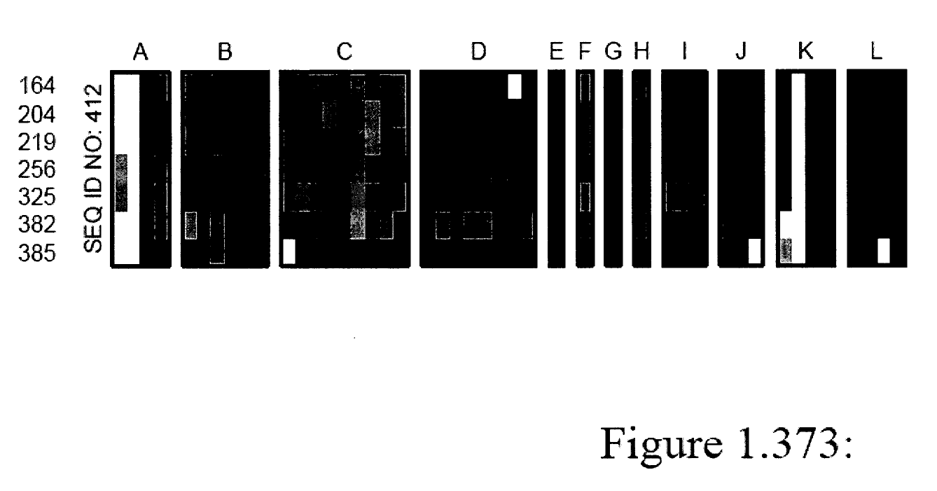

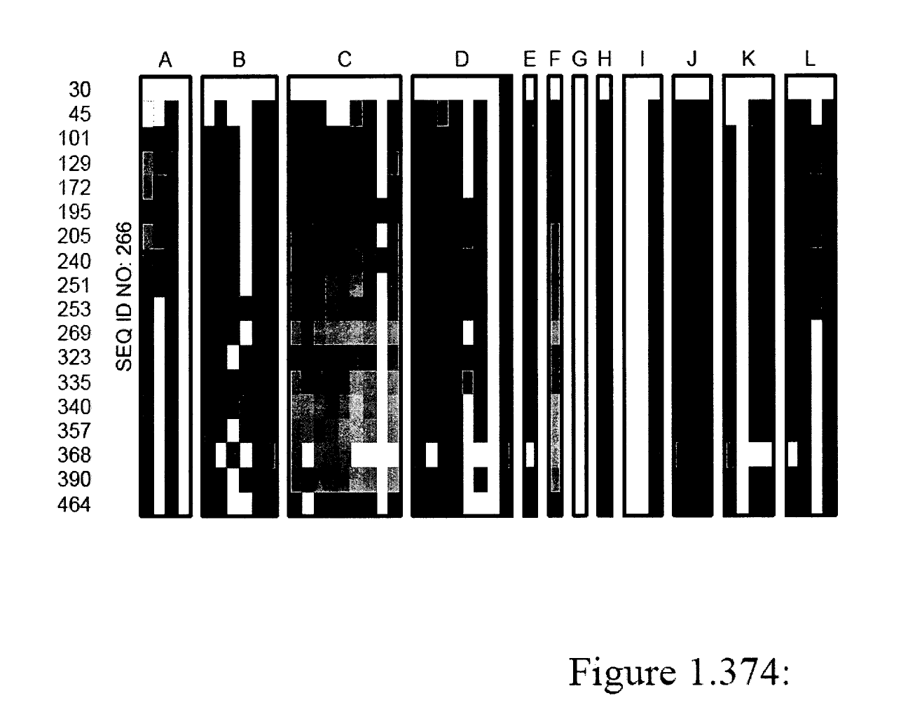

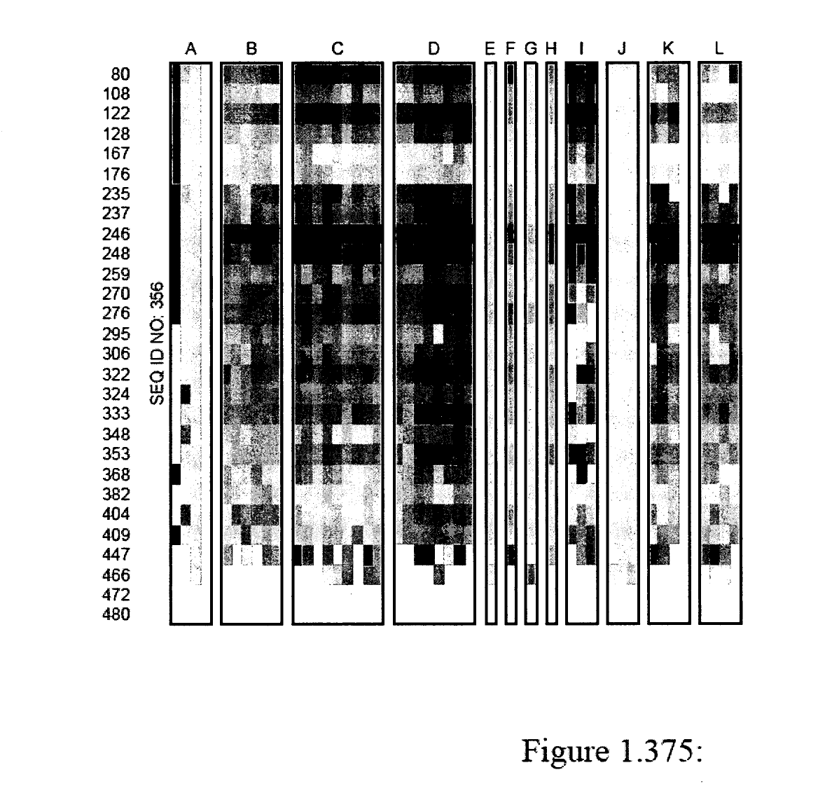

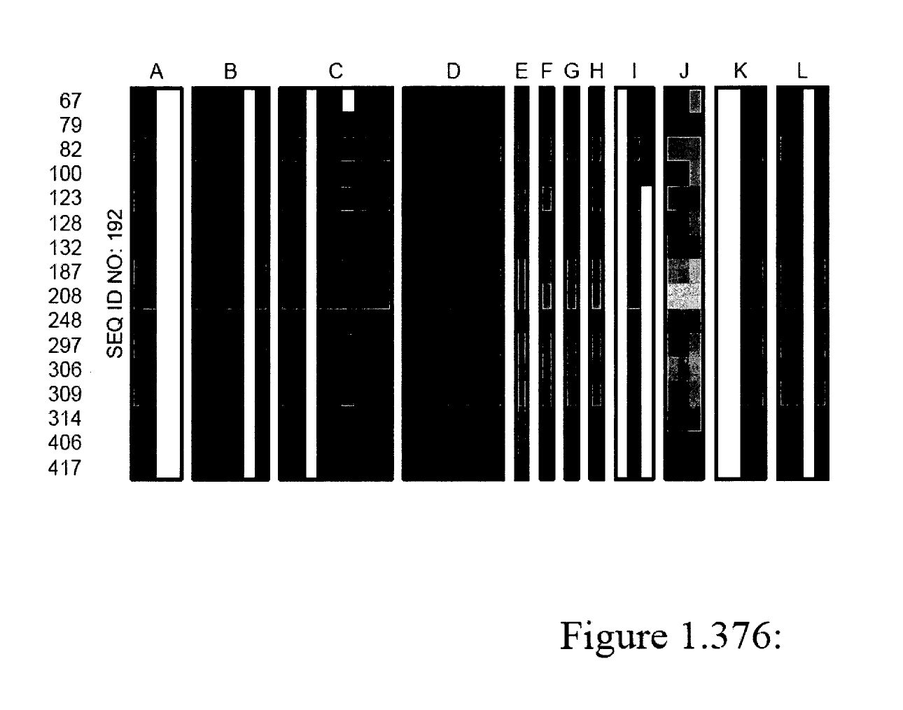

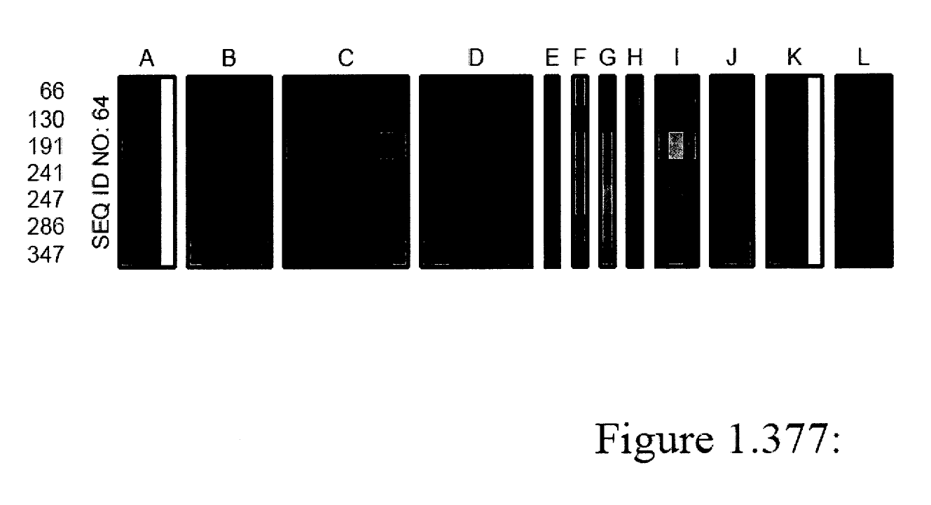

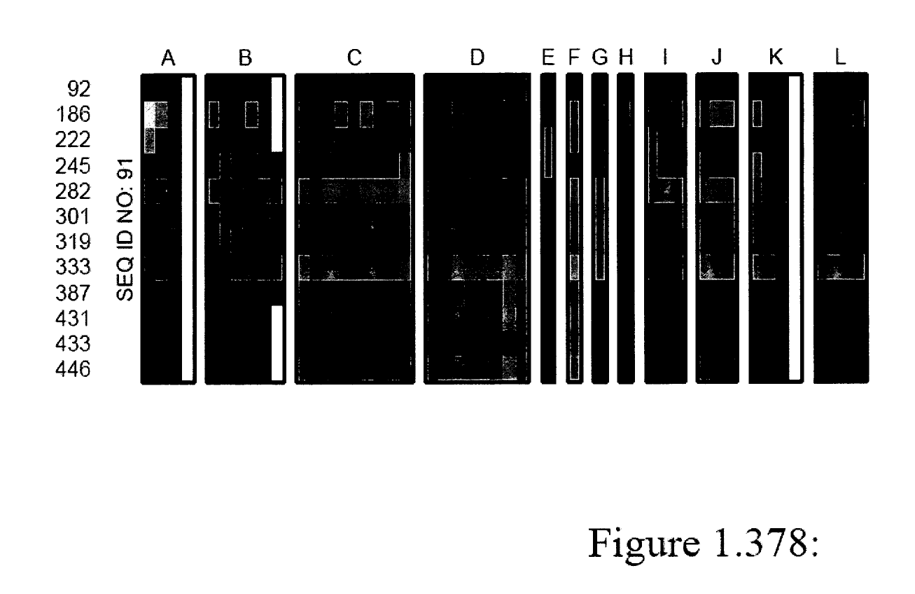

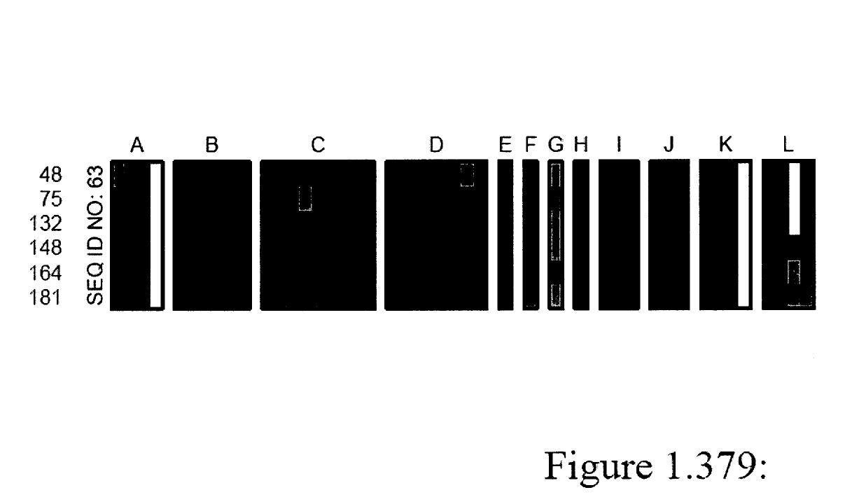

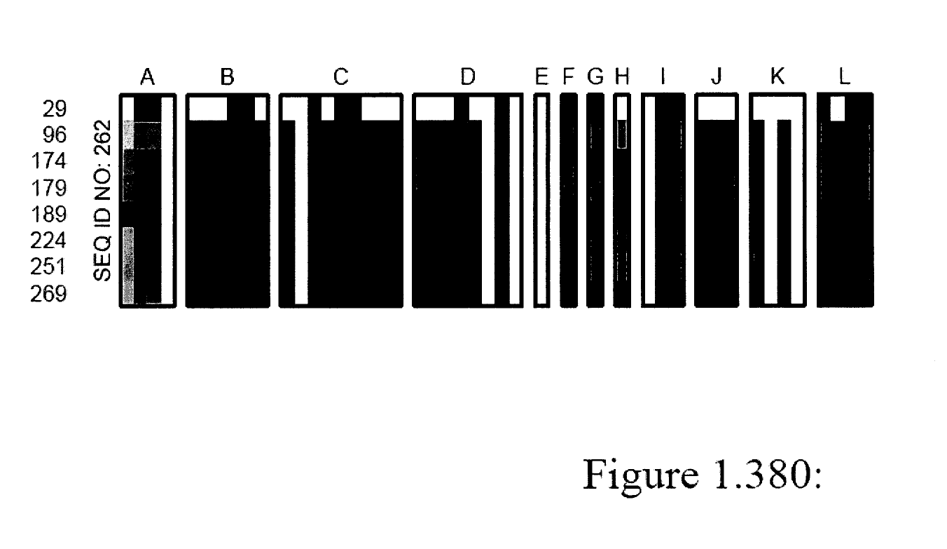

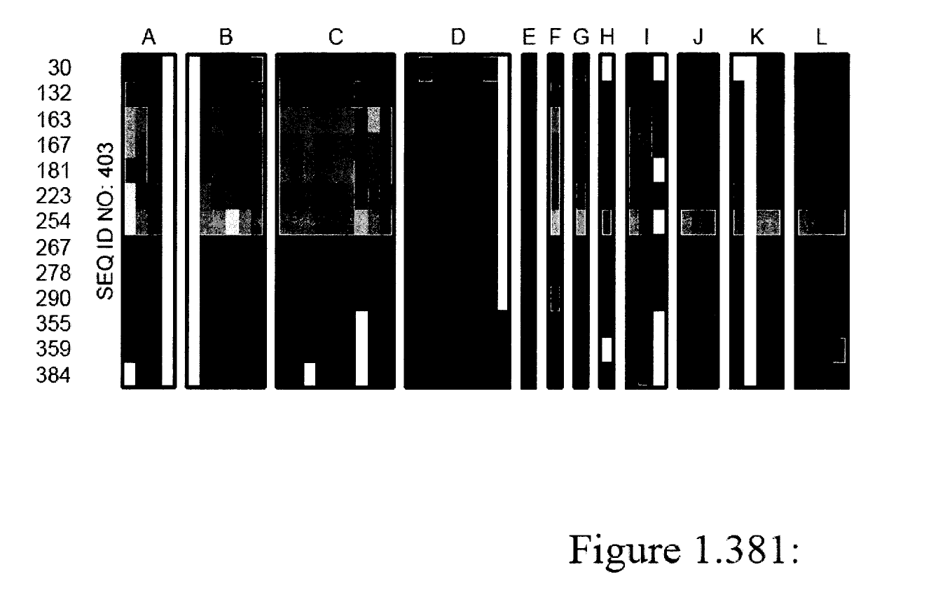

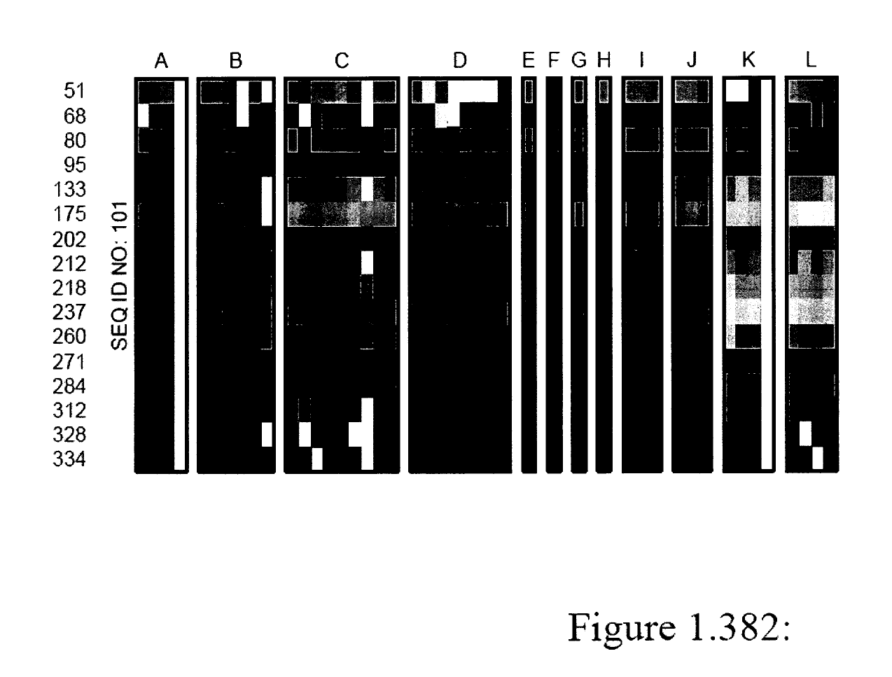

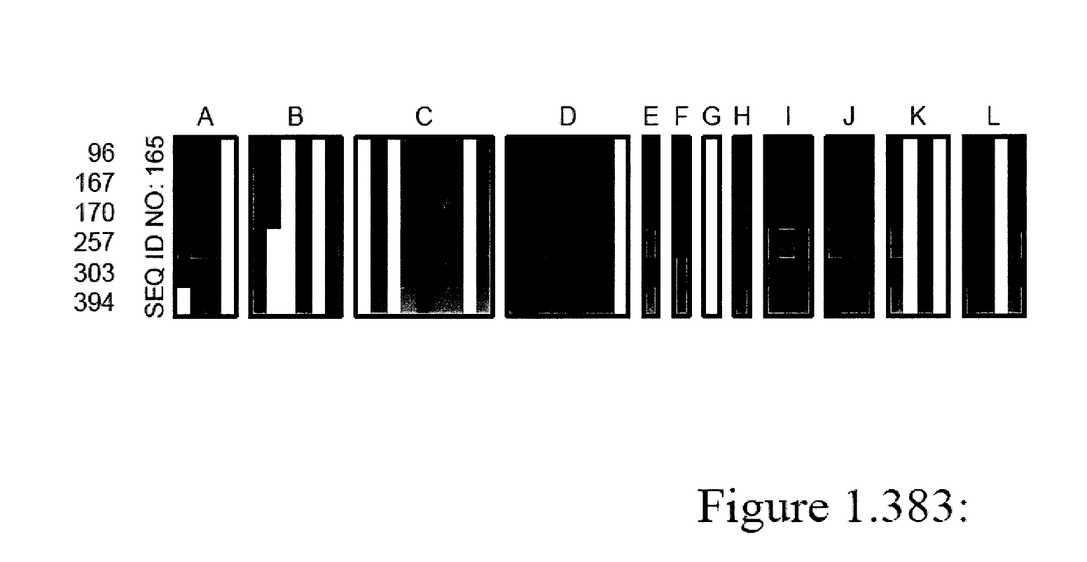

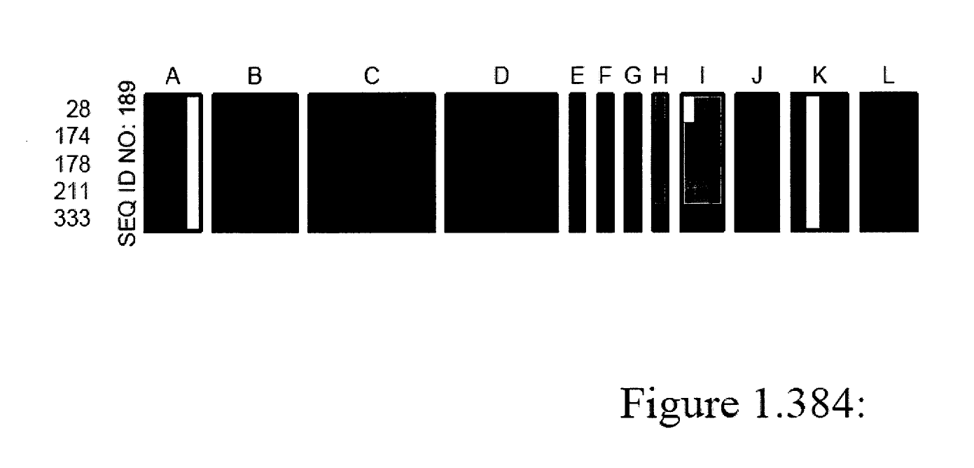

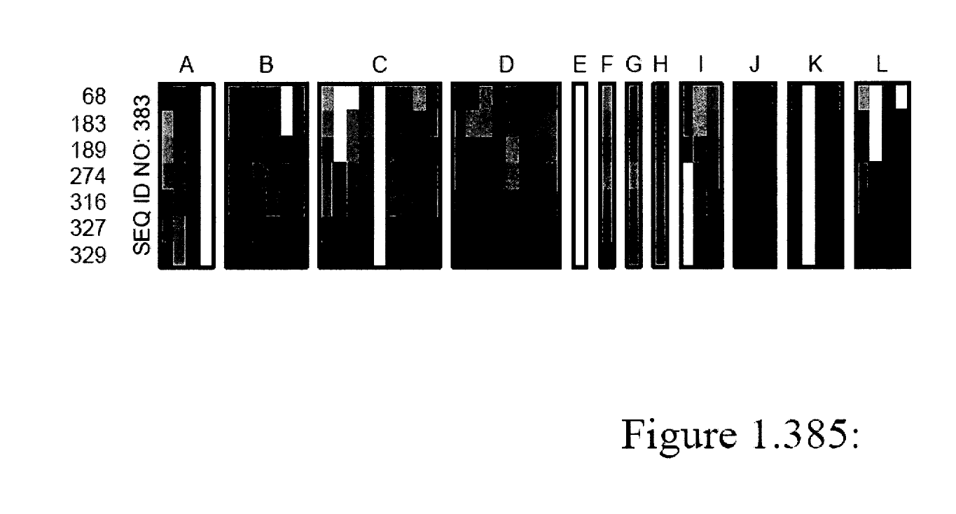

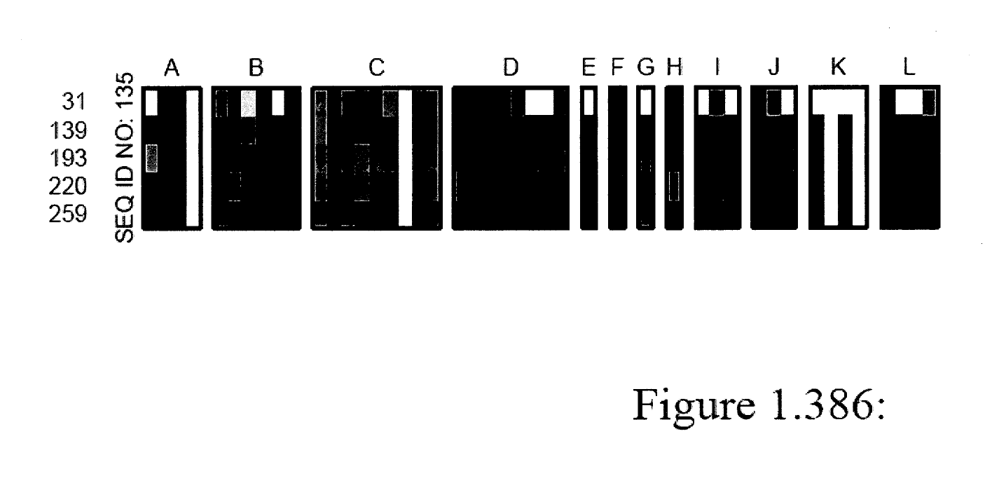

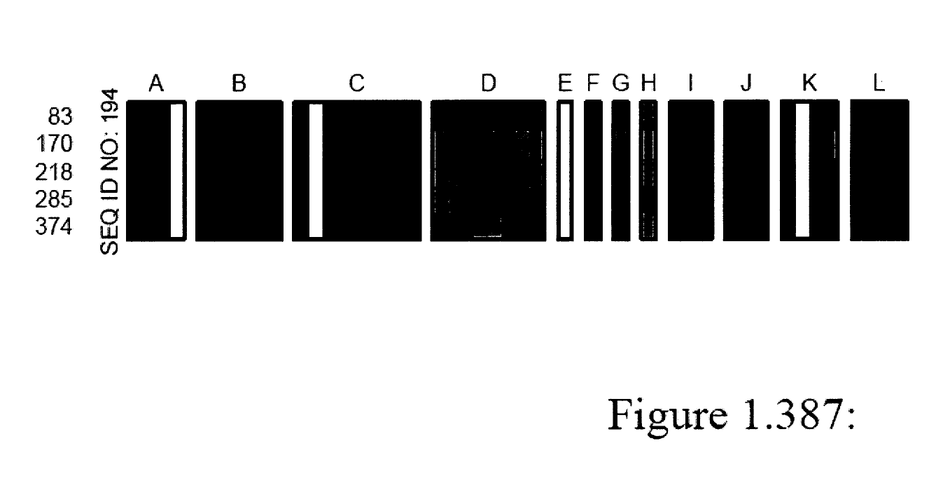

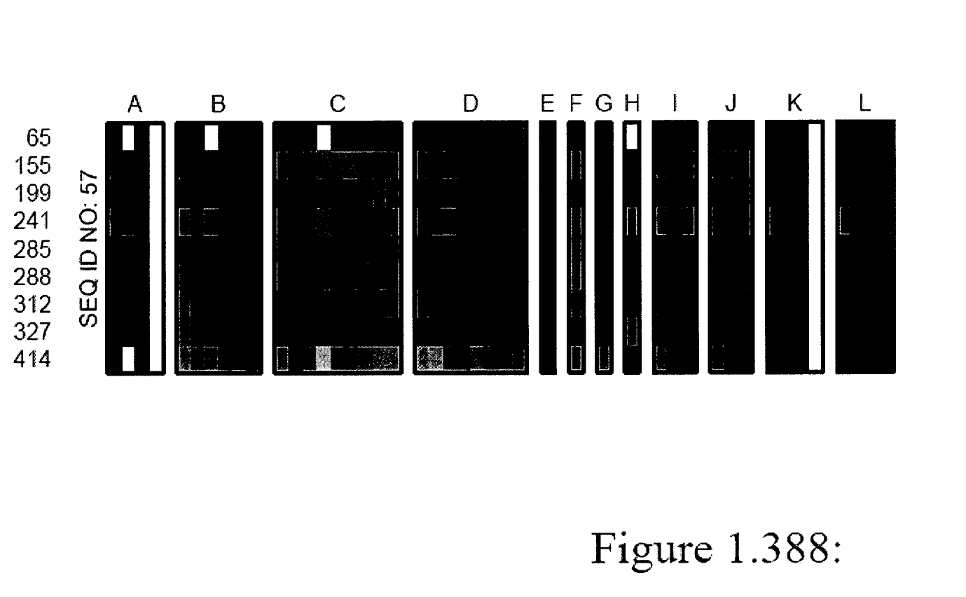

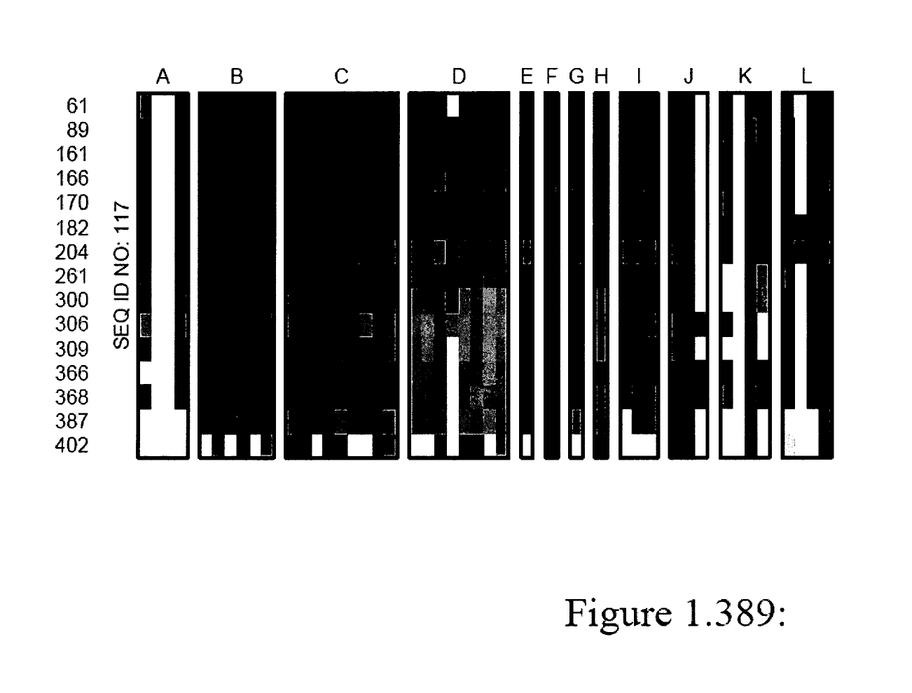

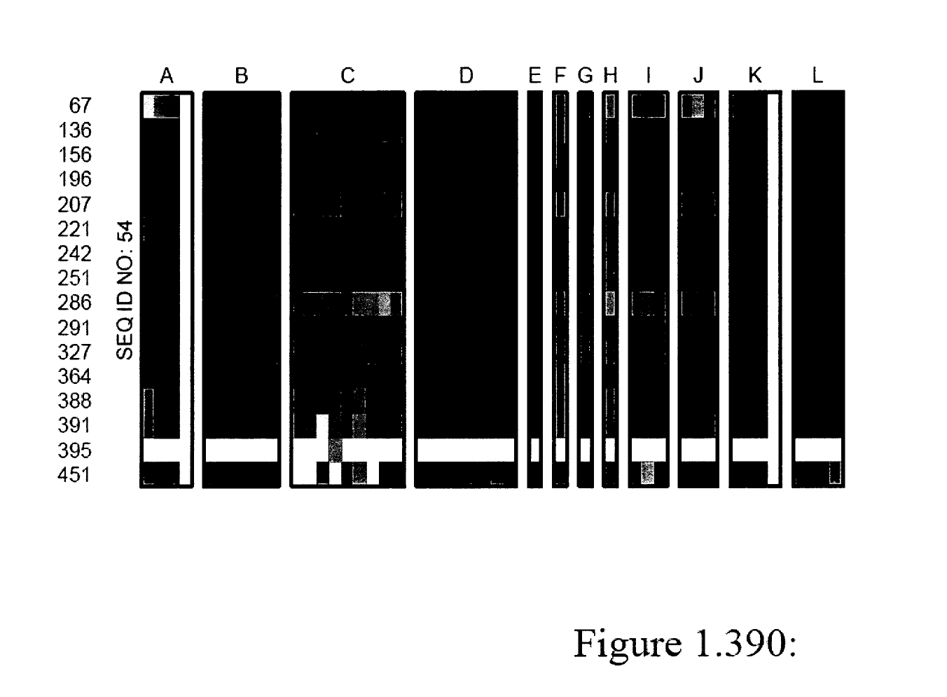

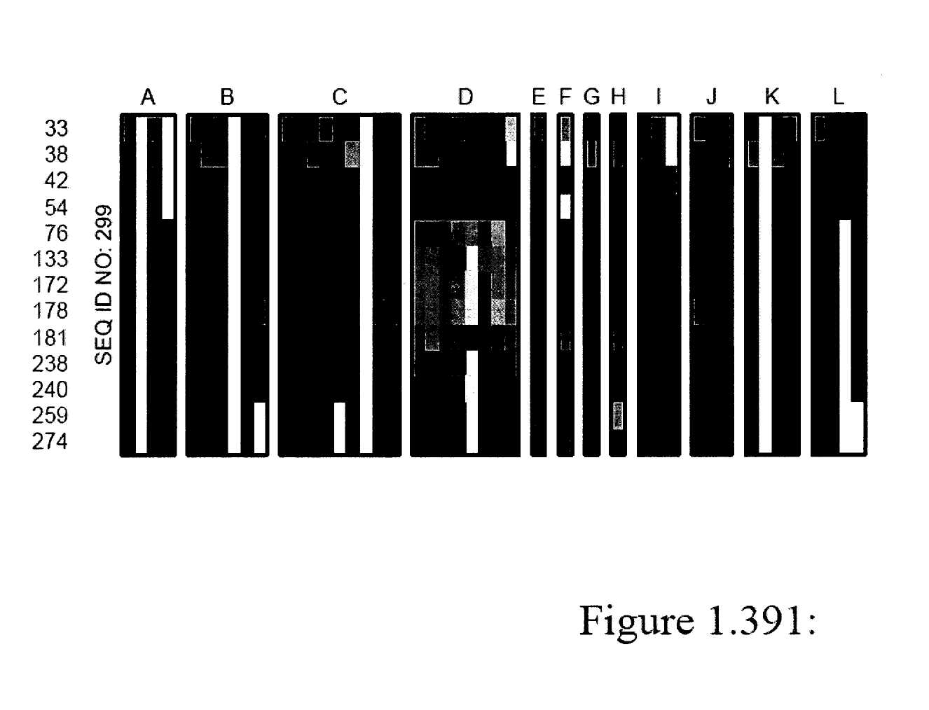

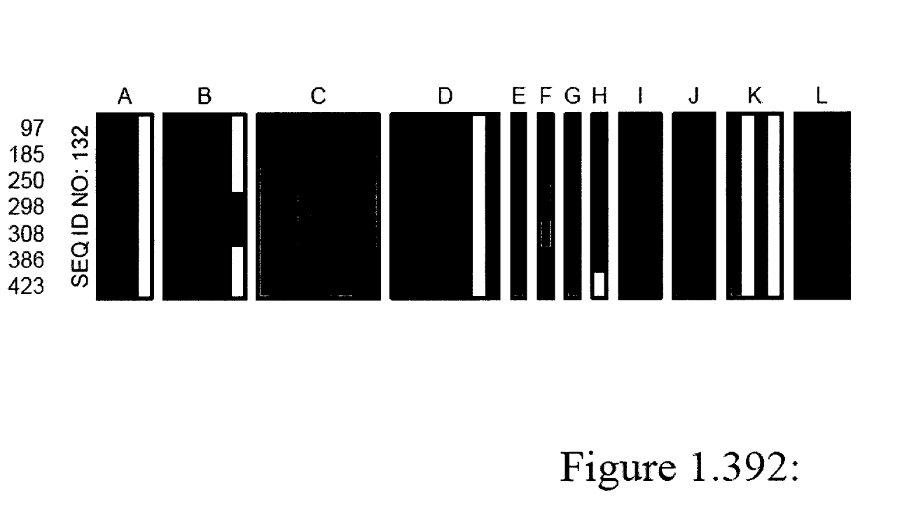

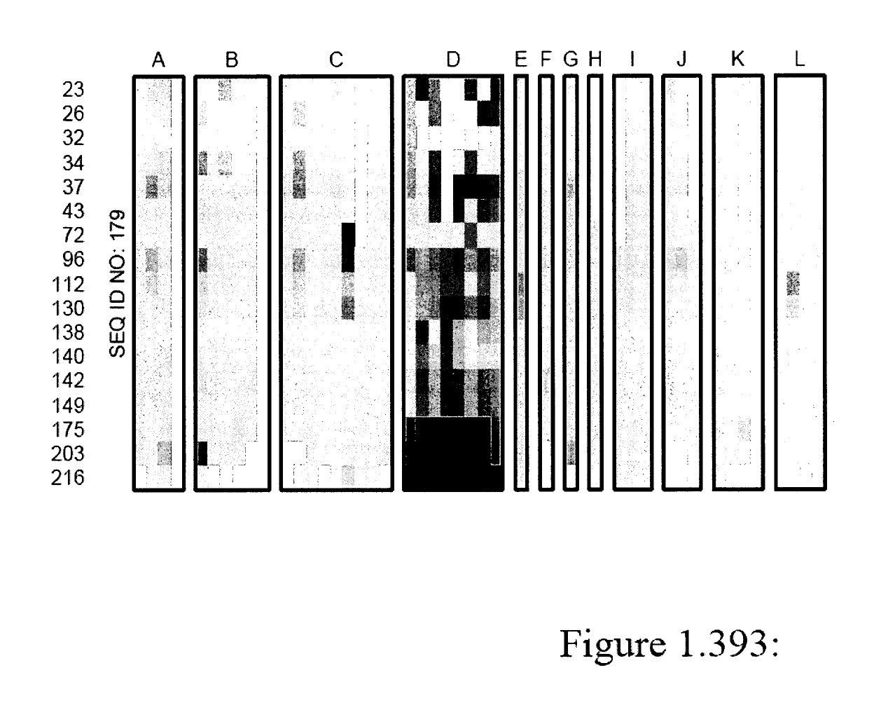

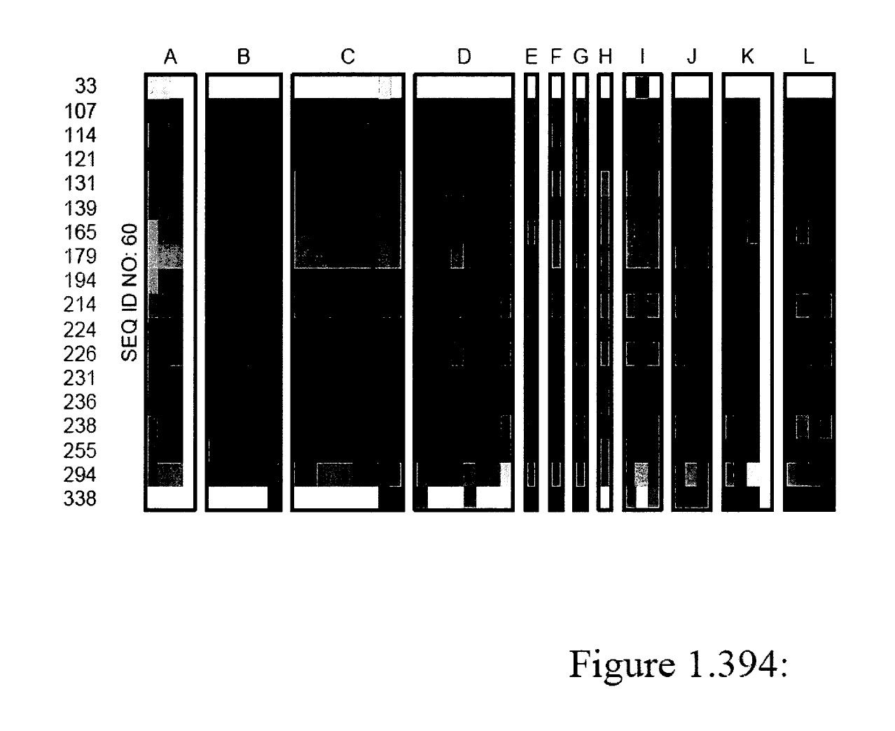

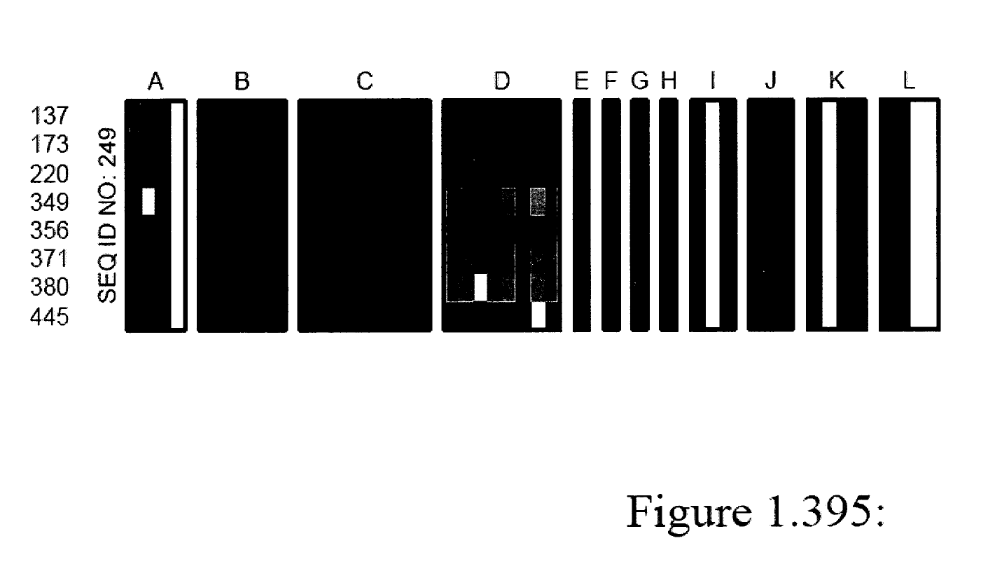

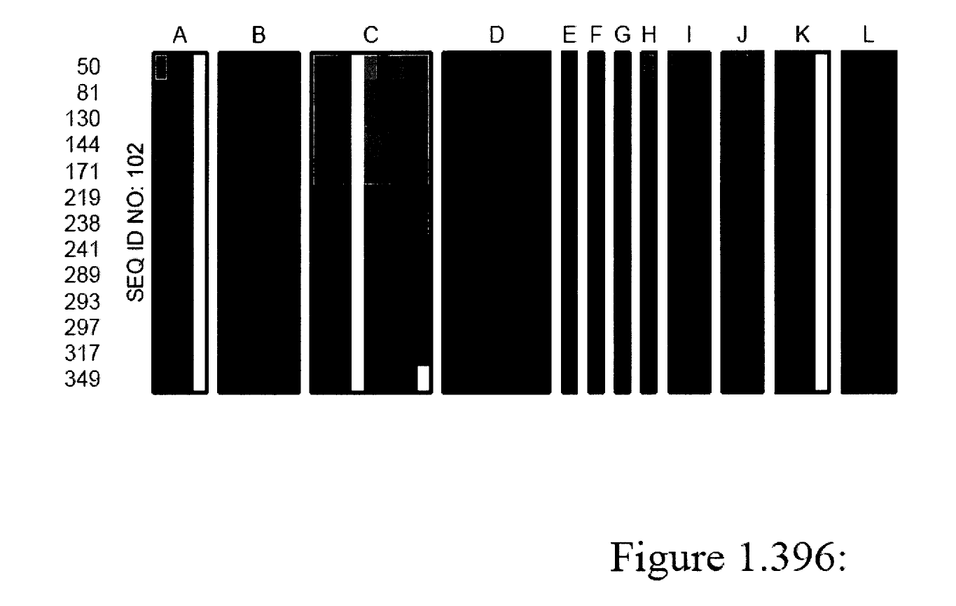

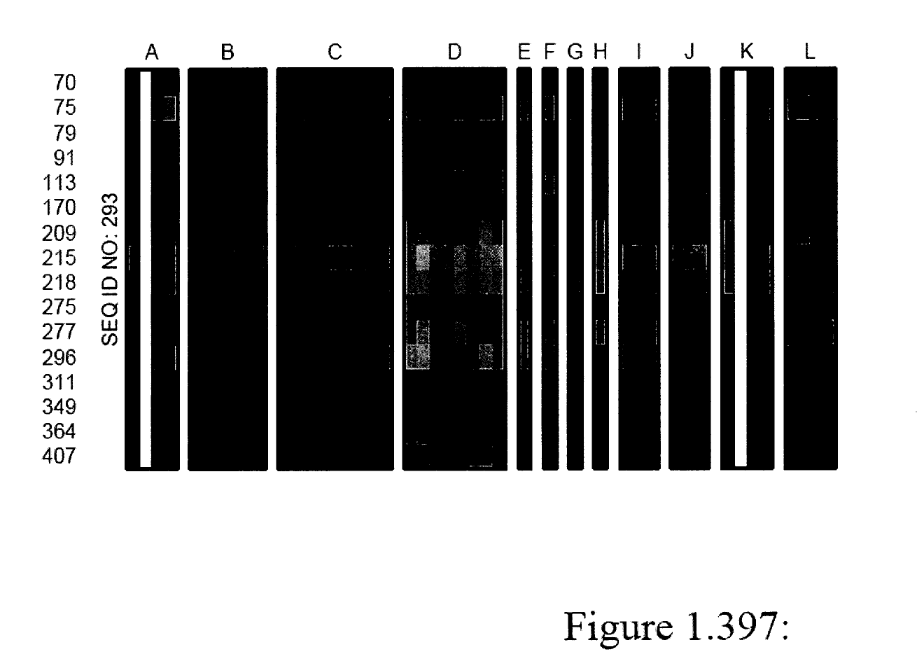

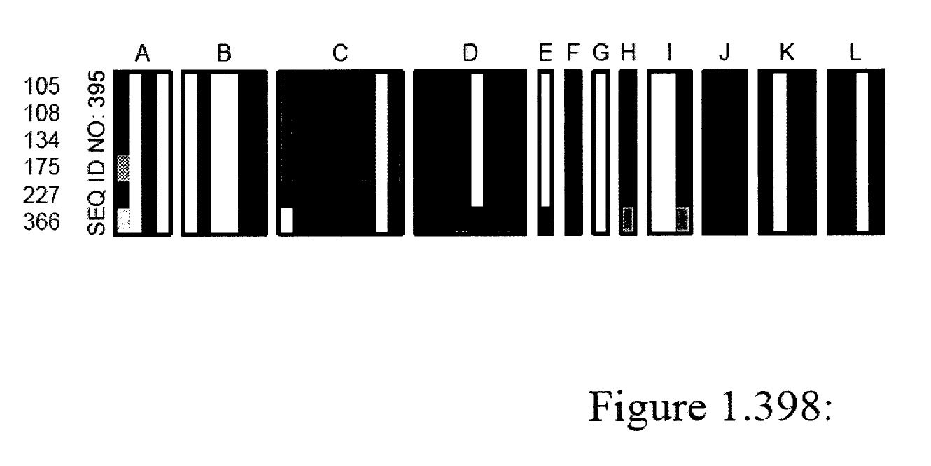

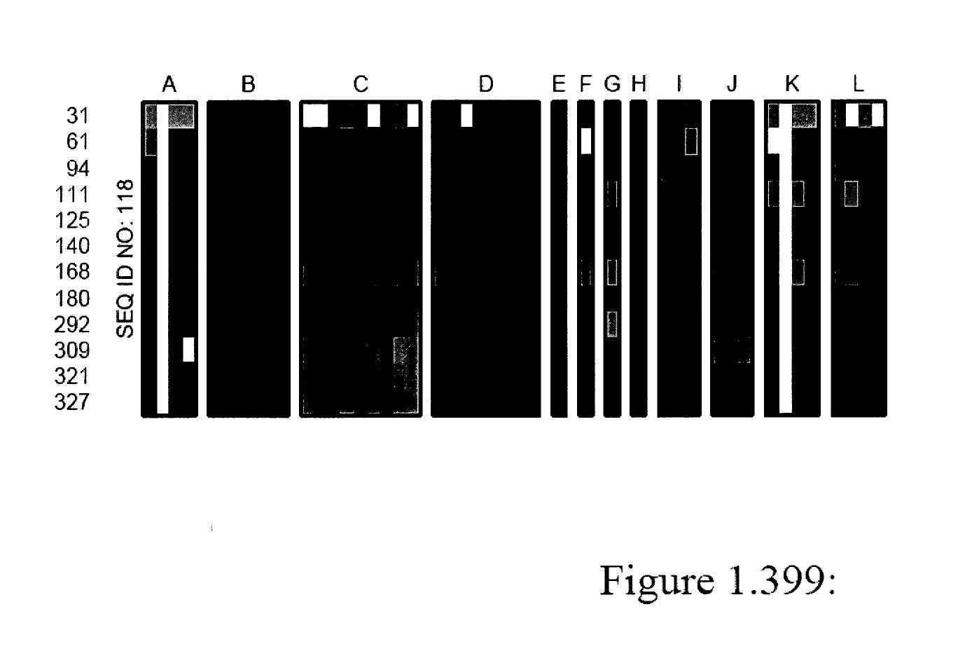

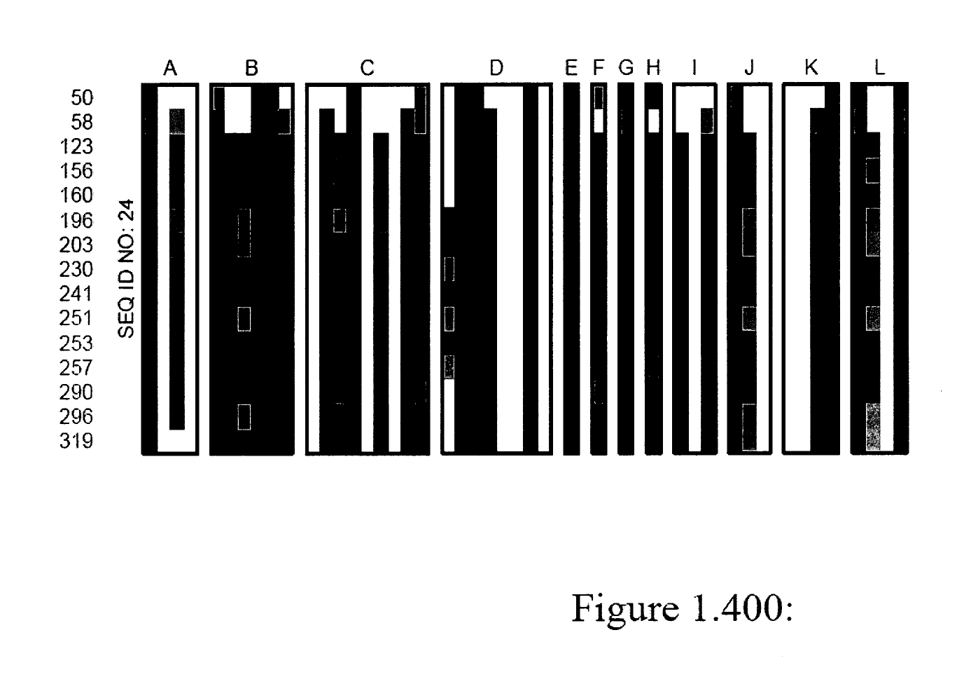

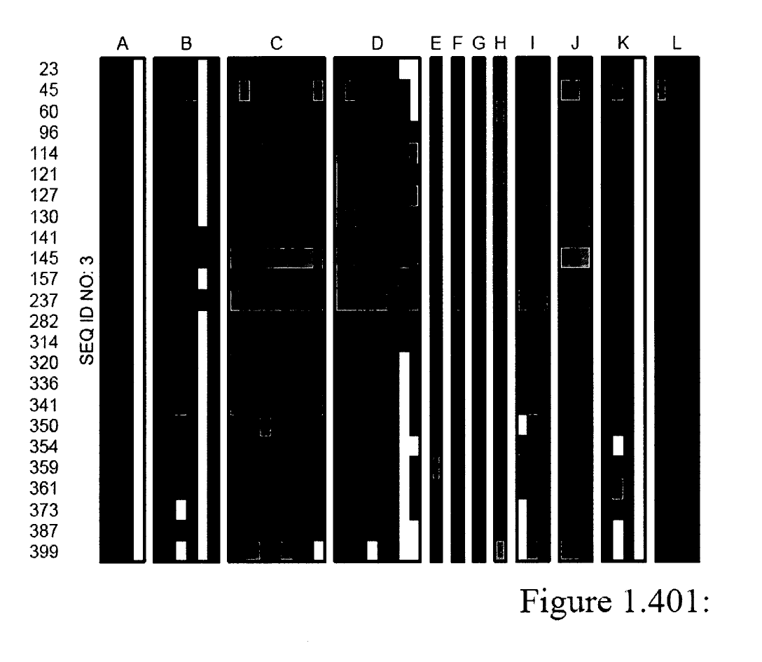

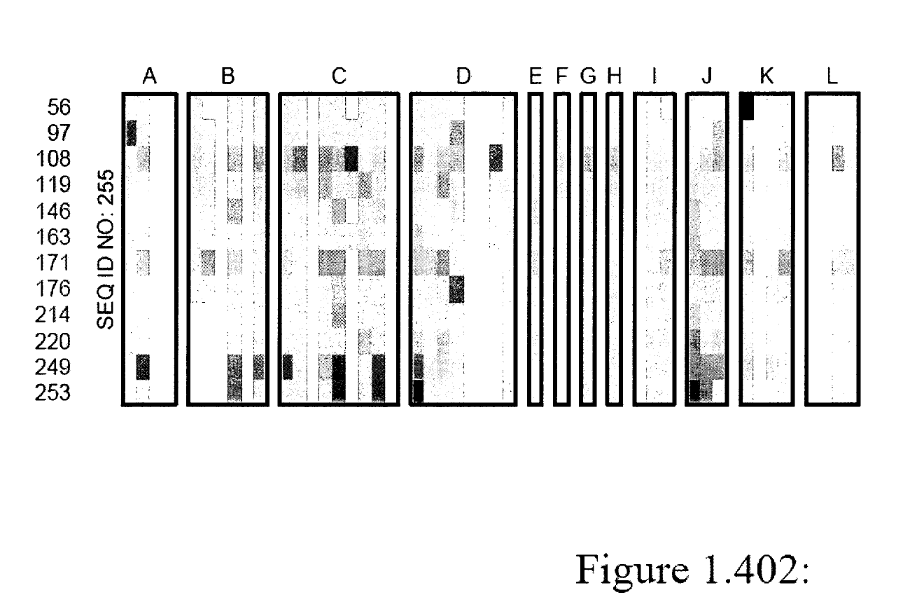

In a preferred embodiment, the herein provided markers of Table 8A specific only for CD8 T-lymphocytes are used for quantifying CD8 T-lymphocytes, in particular for monitoring the immune system of individuals infected with HIV. The periodically determining of the number of CD8 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection. For example but not limited to, the differential methylation of RP4-695O20_B.9 SEQ ID NO: 706 is used. According to FIG. 1.296 and to Table 6, the differential methylation of RP4-695O20_B.9 SEQ ID NO: 706 is a marker for CD8 T-lymphocytes because the CpG dinucleotides of RP4-695O20_B.9 SEQ ID NO: 706 are methylated within the range of 75-100% in CD8 T-lymphocytes, sperm and liver while other tissues show a different extend of methylation. Furthermore CD8 T-lymphocytes can easily be distinguished from sperm or liver morphologically or by means of other markers. Of course, corresponding markers can also be alternatively used. Thereby corresponding markers of RP4-695O20_B.9 SEQ ID NO: 706 are, for example but not limited to, genomic DNA derived from or associated with RP4-695O20_B.9 SEQ ID NO: 706; methylation specifically converted DNA derived from RP4-695O20_B.9 SEQ ID NO: 706; mRNA, cDNA, protein, or peptide each of which derived at least in parts from RP4-695O20_B.9 SEQ ID NO: 706. If the case may be, a person skilled in the art knows how to adjust the presented procedures.

Correspondingly, for example but not limited to, a method for quantifying CD8 T-lymphocytes comprises:

1. Providing a sample comprising genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega). Preferably said sample is a blood sample, plasma sample, or urine sample.
2. Determining the methylation level of at least one CpG position within the sequence of RP4-695O20_B.9 SEQ ID NO: 706 of the provided sample. Thereby a RP4-695O20_B.9 profile specific of said sample is generated. A person skilled in the art knows how to determine the methylation state or the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Deducing the number of CD8 T-lymphocytes from the comparison of the determined RP4-695O20_B.9 profile with one or more RP4-695O20_B.9 reference profiles. Said reference profiles are specific for a defined number of CD8 T-lymphocytes. In addition the reference profiles are obtained from the same sample type as the provided sample. For example but not limited to, blood is the sample type for the provided sample and the reference profiles. In a preferred embodiment, wherein the determined RP4-695O20_B.9 profile matches or is similar to a reference profile specific for a defined number of CD8 T-lymphocytes, the said number of CD8 T-lymphocytes is present in the analyzed sample. In a preferred embodiment, a calibration curve is prepared form the reference profiles. The number of CD8 T-lymphocytes is then deduced by comparing the determined RP4-695O20_B.9 profile with the calibration curve. Of course, other algorithms as the are known in the art are also preferred.

In addition, for example but not limited to it, a method for quantifying CD8 T-lymphocytes comprises:
1. Providing of a sample, comprising genomic DNA;
2. Contacting the genomic DNA or a derivative of it with at least one probe which is specific for at least one differentially methylated CpG position of the marker RP4-695O20_B.9 SEQ ID NO: 706. Said probe is selected from the group comprising antibody; 5-methylcytosine specific antibody (e.g. AbCam Cat. No. ab1884); affinity binding protein; protein binding specifically methylated or unmethylated DNA like MeCP2, MBD1, MBD2, MBD4, Kaiso or any domain thereof; nucleic acid; DNA, RNA, PNA or nucleic acid derivative specific for the methylated sequence. In addition, the probe is labeled directly or indirectly with a dye, protein, enzyme, metal, bead or chemical compound suitable for detection.
3. Performing a detection reaction by means of the probe and/or the label. A person skilled in the art is aware of suitable detection reactions. For example, but not limited to, the detection reaction comprises Rabbit Peroxidase Anti-Peroxidase (PAP) Soluble Complex (Rockland Catalog#: P300-002); radioactive labeled probes; or probes fluorescently labeled like DNA probes coupled with Cy5 (Invitrogen). A person skilled in the art knows further suitable methods for detection.
4. Quantifying the detection reaction in a manner so that the detected signal is indicative for the amount of probe or label and therewith for the number of dead liver cell. A person knows suitable methods for quantification.

Alternatively, for example but not limited to, a method for quantifying CD8 T-lymphocytes comprises:

1. Providing of a sample, the sample comprising one or more cells or genomic DNA. The genomic DNA is purified from said sample, preferably by means of a kit. A person skilled in the art is aware of suitable kits. For example, but not limited to it, the kit for purification of genomic DNA is the DNeasy Tissue Kit (Qiagen) or the Wizard Genomic DNA Purification kit (Promega).
2. Determining the methylation level of at least one CpG position within the sequence of RP4-695O20_B.9 SEQ ID NO: 706 of the provided sample. A person skilled in the art knows how to determine the methylation level of one or more CpG positions. For example, but not limited to, the methylation state or level is determined by means of at least one selected from the group comprising amplification method, PCR method, isothermal amplification method, NASBA method, LCR method, methylation specific amplification method, MSP (Methylation Specific PCR) method, nested MSP method, HeavyMethyl™ method, detection method, methylation specific detection method, bisulfite sequencing method, detection by means of DNA-arrays, detection by means of oligonucleotide microarrays, detection by means of CpG-island-microarrays, detection by means of restriction enzymes, detection by means of methylation sensitive restriction enzymes simultaneous methylation specific amplification and detection method, COBRA method, real-time PCR, HeavyMethyl™ real time PCR method, MSP MethyLight™ method, MethyLight™ method, MethyLight™ Algo™ method, QM method, Headloop MethyLight™ method, HeavyMethyl™ MethyLight™ method, HeavyMethyl™ Scorpion™ method, MSP Scorpion™ method, Headloop Scorpion™ method, methylation sensitive primer extension, Ms-SNuPE (Methylation-sensitive Single Nucleotide Primer Extension) method, and proteins binding specifically methylated or unmethylated DNA like the proteins MeCP2, MBD1, MBD2, MBD4, Kaiso or any domain thereof like but not limited to the CXXC-3 domain of the MBD1 protein or methylation-specific antibodies, e.g. anti-5-methylcytosine antibodies.
3. Quantifying the number of CD8 T-lymphocytes by comparing the determined one or more methylation levels of the sample with the respective herein provided one or more methylation levels of CD8 T-lymphocytes and with at least one methylation level representing the correspondent tissue, group of cells, or cell. Thereby said correspondent tissue, group of cells, or cell is derived from a healthy individual or is completely free of CD8 T-lymphocytes.

### CD4+ Lymphocytes:

In a preferred embodiment, the herein provided markers of Table 8A specific only for CD4 T-lymphocytes are used for quantifying CD4 T-lymphocytes, in particular for monitoring the immune system of individuals infected with HIV. The periodically determining of the number of CD4 T-lymphocytes for patients infected with HIV is a standard procedure in the art. It is necessary to decide whether and when a drug or treatment is necessary, whether a drug or treatment is still effective, and which drug or treatment can be selected. The said is necessary with respect to the HIV infection itself but also with respect to secondary infection. For example but not limited to, the differential methylation of SEQ ID NO: 652 (no gene associated) is used. According to Table 6, the differential methylation of SEQ ID NO: 652 is a marker for CD4 T-lymphocytes because the CpG dinucleotides of SEQ ID NO: 652 are methylated within the range of 25-75% in CD4 T-lymphocytes while other tissues show a different extend of methylation. For example but not limited to, the quantification of CD4 T-lymphocytes is carried out as the above described methods for quantifying CD8 T-lymphocytes wherein the marker RP4-695O20_B.9 SEQ ID NO: 706 is substituted by SEQ ID NO: 652. Of course, corresponding markers can also be alternatively used. Thereby corresponding markers of SEQ ID NO: 652 are, for example but not limited to, genomic DNA derived from or associated with SEQ ID NO: 652; methylation specifically converted DNA derived from SEQ ID NO: 652; mRNA, cDNA, protein, or peptide each of which derived at least in parts from SEQ ID NO: 652. If the case may be, a person skilled in the art knows how to adjust the presented procedures.

All methods named in the above embodiments are known in the art. They are described for example in EP06075376.1 or in PCT/US2006/014667 both of which is hereby incorporated by reference.

While the present invention has been described with specificity in accordance with certain of its preferred embodiments, the following EXAMPLES and TABLES serve only to illustrate the invention and are not intended to limit the invention within the principles and scope of the broadest interpretations and equivalent configurations thereof.

### Tables

TABLE 1
Overview of the genes or genomic regions according ot the present invention, with HUGO
ID and the SEQ ID NO.

| | | SEQ ID NO: |
|---|---|---|
| Gene | HUGO ID | Genomic |
| FLOT1, flotillin 1, ENSG00000137312 | FLOT1 | 413 |
| C6orf25, chromosome 6 open reading frame 25, | C6orf25 | 414 |
| ENSG00000096148 | | |
| VARS, valyl-tRNA synthetase, ENSG00000096171 | VARS | 415 |
| major histocompatibility complex, class II, DP beta 1, | HLA-DPB1 | 416 |
| OTTHUMG00000031076, HLA-DPB1 | | |
| HLA-DRB5, major histocompatibility complex, class II, DR | HLA-DRB5 | 417 |
| beta 5, OTTHUMG00000031027 | | |
| COL11A2, collagen, type XI, alpha 2, | COL11A2 | 418 |
| OTTHUMG00000031036 | | |
| PRAME, Melanoma antigen preferentially expressed in | PRAME | 419 |
| tumors (Preferentially expressed antigen of melanoma) (OPA- | | |
| interacting protein 4) (OIP4), ENSG00000185686 | | |
| ZNRF3 protein (Fragment), ENSG00000183579, ZNRF3 zinc | ZNRF3 | 420 |
| and ring finger 3 (ZNRF3) | | |
| AP000357.2 (Vega gene ID), Pseudogene | AP000357.2 (Vega | 421 |
| | gene ID) | |
| AP000357.3 (Vega gene ID), Pseudogene | AP000357.3 (Vega | 422 |
| | gene ID) | |
| solute carrier family 7 (cationic amino acid transporter, y+ | SLC7A4 | 423 |
| system), member 4, OTTHUMG00000030129, SLC7A4 | | |
| Myosin-18B (Myosin XVIIIb), ENSG00000133454, | MYO18B | 424 |
| MYO18B | | |
| Q6ICL0_HUMAN (Predicted UniProt/TrEMBL ID), | Q6ICL0_HUMAN | 425 |
| hypothetical protein FLJ3257; ENSG00000184004 | (Predicted | |
| | UniProt/TrEMBL | |
| | ID) | |
| FBLN1; fibulin 1; ENSG00000077942 | FBLN1 | 426 |
| CYP2D6; cytochrome P450, family 2, subfamily D, | CYP2D6 | 427 |
| polypeptide 6; ENSG00000100197 | | |
| AC008132.9 (Vega gene ID); Pseudogene; | AC008132.9 (Vega | 428 |
| OTTHUMG00000030688 | gene ID) | |
| glycoprotein Ib (platelet), beta polypeptide, | GP1BB | 429 |
| OTTHUMT00000075045, GP1BB-001 | | |
| no gene associated | | 430 |
| AC006548.8 (Vega gene ID) | AC006548.8 (Vega | 431 |
| | gene ID) | |
| OTTHUMG00000030650, AC005399.2, putative processed | AC005399.2 (Vega | 432 |
| transcribed | gene ID) | |
| topoisomerase (DNA) III beta, OTTHUMG00000030764, | TOP3B | 433 |
| TOP3B | | |
| no gene associated | | 434 |
| KB-1269D1.3 (Vega gene ID); Pseudogene; | KB-1269D1.3 (Vega | 435 |
| OTTHUMG00000030694 | gene ID) | |
| GPR24; G protein-coupled receptor 24; ENSG00000128285 | GPR24 | 436 |
| GAL3ST1; galactose-3-O-sulfotransferase 1; | GAL3ST1 | 437 |
| ENSG00000128242 | | |
| Cat eye syndrome critical region protein 5 precursor, | CECR5 | 438 |
| ENSG00000069998, CECR5 and (head to head), | | |
| OTTHUMG00000030478, AC006946.7 | | |
| HORMAD2; HORMA domain containing 2; | HORMAD2 | 439 |
| ENSG00000176635 | | |
| OTTHUMG00000030922, RP3-438O4.2 | RP3-438O4.2 | 440 |
| | (Vega_gene ID) | |
| NP_997357.1 (RefSeq peptide ID); ENSG00000169668 | NP_997357.1 | 441 |
| | (RefSeq peptide ID) | |
| OTTHUMG00000030574, AP000357.3, novel pseudogene | AP000357.2 (Vega | 442 |
| | gene ID) | |
| LA16c-4G1.2 (Vega gene ID); Pseudogene; | LA16c-4G1.2 (Vega | 443 |
| OTTHUMG00000030832 | gene ID) | |
| KB-226F1.11 (Vega gene ID), embryonic marker, | KB-226F1.11 (Vega | 444 |
| OTTHUMG00000030123 | gene ID) | |
| OTTHUMG00000030780, CTA-373H7.4, novel pseudogene | CTA-373H7.4 (Vega | 445 |
| | gene ID) | |
| RP1-47A17.8 (Vega gene ID); OTTHUMG00000030878 | RP1-47A17.8 (Vega | 446 |
| | gene ID); | |
| RP4-539M6.7 (Vega gene ID); Pseudogene; | RP4-539M6.7 (Vega | 447 |
| OTTHUMG00000030918 | gene ID) | |
| CSDC2; cold shock domain containing C2, RNA binding; | CSDC2 | 448 |
| ENSG00000172346 | | |
| Gamma-parvin, ENSG00000138964, PARVG | PARVG | 449 |
| OTTHUMG00000030167, CTA-243E7.3 | CTA-243E7.3 (Vega | 450 |
| | gene ID) | |
| Oncostatin M precursor (OSM), ENSG00000099985, OSM | OSM | 451 |
| Oncostatin M precursor (OSM), ENSG00000099985, OSM | OSM | 452 |
| Myosin-18B (Myosin XVIIIb), ENSG00000133454, | MYO18B | 453 |
| MYO18B | | |
| Q6ICL0_HUMAN (Predicted UniProt/TrEMBL ID), | Q6ICL0_HUMAN | 454 |
| hypothetical protein FLJ3257; ENSG00000184004 | (Predicted | |
| | UniProt/TrEMBL | |
| | ID) | |
| OTTHUMG00000030140, CTA-299D3.6 | CTA-299D3.6 (Vega | 455 |
| | gene ID) | |
| GALR3; galanin receptor 3; ENSG00000128310 | GALR3 | 456 |
| GALR3; galanin receptor 3; ENSG00000128310 | GALR3 | 457 |
| IL2RB; interleukin 2 receptor, beta; ENSG00000100385 | IL2RB | 458 |
| CTA-343C1.3 (Vega gene ID); Putative Processed transcript; | CTA-343C1.3 (Vega | 459 |
| OTTHUMG00000030151 | gene ID) | |
| CTA-941F9.6 (Vega_gene ID) | CTA-941F9.6 | 460 |
| | (Vega_gene ID) | |
| CTA-941F9.6 (Vega_gene ID) | CTA-941F9.6 | 461 |
| | (Vega_gene ID) | |
| LL22NC03-121E8.1 (Vega gene ID); Novel Protein coding; | LL22NC03-121E8.1 | 462 |
| OTTHUMG00000030676 | (Vega gene ID) | |
| Cytohesin-4, ENSG00000100055, PSCD4 | PSCD4 | 463 |
| RP4-754E20_A.4 (Vega gene ID); Putative Processed | RP4-754E20_A.4 | 464 |
| transcript; OTTHUMG00000030716 | (Vega gene ID) | |
| PIB5PA; phosphatidylinositol (4,5) bisphosphate 5- | PIB5PA | 465 |
| phosphatase, A; ENSG00000185133; embryonic marker | | |
| no gene associated | | 466 |
| PLA2G3; ENSG00000100078; phospholipase A2, group III | PLA2G3 | 467 |
| PLA2G3; ENSG00000100078; phospholipase A2, group III | PLA2G3 | 468 |
| DGCR2; DiGeorge syndrome critical region gene 2; | DGCR2 | 469 |
| ENSG00000070413 | | |
| TCN2; transcobalamin II; macrocytic anemia; | TCN2 | 470 |
| ENSG00000185339 | | |
| IGLL1; immunoglobulin lambda-like polypeptide 1; | IGLL1 | 471 |
| ENSG00000128322 | | |
| RP1-29C18.7 (Vega gene ID); Novel Processed transcript; | RP1-29C18.7 (Vega | 472 |
| OTTHUMG00000030424 | gene ID) | |
| IGLC1; immunoglobulin lambda constant 1 (Mcg marker); | IGLC1 | 473 |
| ENSG00000100208 | | |
| APOBEC3B; apolipoprotein B mRNA editing enzyme, | APOBEC3B | 474 |
| catalytic polypeptide-like 3B; ENSG00000179750 | | |
| CRYBB1; crystallin, beta B1; ENSG00000100122 | CRYBB1 | 475 |
| CRYBA4; crystallin, beta A4; ENSG00000196431 | CRYBA4 | 476 |
| sushi domain containing 2, ENSG00000099994, SUSD2 | SUSD2 | 477 |
| sushi domain containing 2, ENSG00000099994, SUSD2 | SUSD2 | 478 |
| OTTHUMG00000030870, Putative Processed transcript, | CTA-503F6.1 (Vega | 479 |
| CTA-503F6.1 | gene ID) | |
| embryonic marker, OTTHUMG00000030800, KB-1323B2.3 | KB-1323B2.3 (Vega | 480 |
| | gene ID) | |
| no gene associated | | 481 |
| IGLV1-44; immunoglobulin lambda variable 1-44; | IGLV1-44 | 482 |
| ENSG00000186751 | | |
| IGLV1-44; immunoglobulin lambda variable 1-44; | IGLV1-44 | 483 |
| ENSG00000186751 | | |
| OTTHUMG00000030922, RP3-438O4.2 | RP3-438O4.2 | 484 |
| | (Vega_gene ID) | |
| OTTHUMG00000030922, RP3-438O4.2 | RP3-438O4.2 | 485 |
| | (Vega_gene ID) | |
| APOL4; apolipoprotein L, 4; ENSG00000100336 | APOL4 | 486 |
| OTTHUMG00000030852, RP4-756G23.1, novel processed | RP4-756G23.1 | 487 |
| transcript | (Vega gene ID) | |
| ENSG00000100399, Q96E60_HUMAN | Q96E60_HUMAN | 488 |
| | (Predicted | |
| | UniProt/TrEMBL | |
| | ID) | |
| Neutrophil cytosol factor 4 (NCF-4) (Neutrophil NADPH | NCF4 | 489 |
| oxidase factor 4) (p40-phox) (p40phox)., ENSG00000100365, | | |
| NCF4 | | |
| Neutrophil cytosol factor 4 (NCF-4) (Neutrophil NADPH | NCF4 | 490 |
| oxidase factor 4) (p40-phox) (p40phox)., ENSG00000100365, | | |
| NCF4 | | |
| Somatostatin receptor type 3 (SS3R) (SSR-28), D | SSTR3 | 491 |
| ENSG00000183473, SSTR3 | | |
| Somatostatin receptor type 3 (SS3R) (SSR-28), D | SSTR3 | 492 |
| ENSG00000183473, SSTR3 | | |
| Bcl-2 interacting killer (Apoptosis inducer NBK) (BP4) | BIK | 493 |
| (BIP1)., ENSG00000100290, BIK | | |
| GAS2-like protein 1 (Growth arrest-specific 2-like 1) (GAS2- | GAS2L1 | 494 |
| related protein on chromosome 22) (GAR22 protein), | | |
| ENSG00000185340, GAS2L1 | | |
| RP3-355C18.2 (Vega gene ID) | RP3-355C18.2 | 495 |
| | (Vega gene ID) | |
| SOX10; SRY (sex determining region Y)-box 10; | SOX10 | 496 |
| ENSG00000100146 | | |
| Gamma-parvin ENSG00000138964 | PARVG | 497 |
| Caspase recruitment domain protein 10 (CARD-containing | CARD10 | 498 |
| MAGUK protein 3) (Carma 3). ENSG00000100065, CARD10 | | |
| ENSG00000100101, NP_077289.1 | NP_077289.1 | 499 |
| HTF9C; HpaII tiny fragments locus 9C; ENSG00000099899 | HTF9C_HUMAN | 500 |
| | (UniProt/Swiss-Prot | |
| | ID) | |
| Oncostatin M precursor (OSM), ENSG00000099985, OSM | OSM | 501 |
| CTA-407F11.4 (Vega gene ID); Novel Processed transcript; | CTA-407F11.4 | 502 |
| OTTHUMG00000030804 | (Vega gene ID) | |
| Q6ICL0_HUMAN (Predicted UniProt/TrEMBL ID), | Q6ICL0_HUMAN | 503 |
| hypothetical protein FLJ3257; ENSG00000184004 | (Predicted | |
| | UniProt/TrEMBL | |
| | ID) | |
| CTA-989H11.2 (Vega gene ID); Putative Processed transcript; | CTA-989H11.2 | 504 |
| OTTHUMG00000030141 | (Vega gene ID) | |
| transmembrane protease, serine 6 | TMPRSS6 | 505 |
| HMG2L1; high-mobility group protein 2-like 1; | HMG2L1 | 506 |
| ENSG00000100281 | | |
| NP_001017964.1 (RefSeq peptide ID); hypothetical protein | NP_001017964.1 | 507 |
| LOC150223; ENSG00000161179 | (RefSeq peptide ID) | |
| Platelet-derived growth factor B chain precursor (PDGF B- | PDGFB | 508 |
| chain), ENSG00000100311, PDGFB | | |
| OTTHUMG00000030815, CTA-384D8.15 | CTA-384D8.15 | 509 |
| | (Vega gene ID) | |
| MGAT3; mannosyl (beta-1,4-)-glycoprotein beta-1,4-N- | MGAT3 | 510 |
| acetylglucosaminyltransferase; ENSG00000128268 | | |
| Ceramide kinase (EC 2.7.1.138), Acylsphingosine kinase, | CERK | 511 |
| CERK, Lipid kinase 4, LK4, ENSG00000100422, CERK | | |
| Reticulon 4 receptor precursor (Nogo receptor) (NgR) (Nogo- | RTN4R | 512 |
| 66 receptor), ENSG00000040608, RTN4R | | |
| UNC84B; unc-84 homolog B (C. Elegans); | UNC84B | 513 |
| ENSG00000100242 | | |
| RABL4; RAB, member of RAS oncogene family-like 4; | RABL4 | 514 |
| ENSG00000100360 | | |
| Cadherin EGF LAG seven-pass G-type receptor 1 precursor | CELSR1 | 515 |
| (Flamingo homolog 2) (hFmi2), ENSG00000075275, | | |
| CELSR1 | | |
| OTTHUMG00000030326, LL22NC03-5H6.1 | LL22NC03-5H6.1 | 516 |
| | (Vega gene ID) | |
| OTTHUMG00000030656, RP3-515N1.6 | RP3-515N1.6 (Vega | 517 |
| | gene ID) | |
| SMTN; smoothelin; ENSG00000183963 | SMTN | 518 |
| ZNRF3 protein (Fragment), ENSG00000183579, ZNRF3 zinc | ZNRF3 | 519 |
| and ring finger 3 (ZNRF3) | | |
| OTTHUMG00000030700, GRB2-related adaptor protein 2, | GRAP2 | 520 |
| GRAP2 | | |
| CAP-binding protein complex interacting protein 1 isoform a | NP_073622.2 | 521 |
| Source: RefSeq_peptide NP_073622 | (RefSeq peptide ID) | |
| SAM50_HUMAN (UniProt/Swiss-Prot ID), | SAMM50 | 522 |
| ENSG00000100347, SAM50-like protein CGI-51; sorting | | |
| and assembly machinery component 50 homolog (S. Cerevisiae) | | |
| SULT4A1; sulfotransferase family 4A, member 1; | SULT4A1 | 523 |
| ENSG00000130540 | | |
| TIMP3; TIMP metallopeptidase inhibitor 3 (Sorsby fundus | TIMP3 | 524 |
| dystrophy, pseudoinflammatory); ENSG00000100234 | | |
| T-box transcription factor TBX1 (T-box protein 1) (Testis- | TBX1 | 525 |
| specific T-box protein), ENSG00000184058, TBX1, TBX1 | | |
| is involved in heart development-Great | | |
| ENSG00000186732, metallophosphoesterase domain | MPPED1 | 526 |
| containing 1, NM_001585.2 | | |
| ENSG00000188511, NP_942148.1 novel Gene hypothetical | NP_942148.1 | 527 |
| protein LOC348645 | (RefSeq peptide ID) | |
| Cdc42 effector protein 1, ENSG00000128283, CDC42EP1 | CDC42EP1 | 528 |
| RPL3; ribosomal protein L3; ENSG00000100316 | RPL3 | 529 |
| APOL2; apolipoprotein L, 2; ENSG00000128335 | APOL2 | 530 |
| RAC2; ras-related C3 botulinum toxin substrate 2 (rho family, | RAC2 | 531 |
| small GTP binding protein Rac2); ENSG00000128340 | | |
| OTTHUMP00000028917, ENSG00000100399, | Q96E60_HUMAN | 532 |
| Q96E60_HUMAN | (Predicted | |
| | UniProt/TrEMBL | |
| | ID) | |
| Neutrophil cytosol factor 4 (NCF-4) (Neutrophil NADPH | NCF4 | 533 |
| oxidase factor 4) (p40-phox) (p40phox)., ENSG00000100365, | | |
| NCF4 | | |
| XP_371837.1 (RefSeq peptide predicted ID); PREDICTED: | XP_371837.1 | 534 |
| similar to oxidoreductase UCPA Source: | (RefSeq peptide | |
| RefSeq_peptide_predicted XP_371837; ENSG00000168768 | predicted ID) | |
| triggering receptor expressed on myeloid cells-like 2, | TREML2 | 535 |
| ENSG00000112195, TREML2 | | |
| TREML1; triggering receptor expressed on myeloid cells-like | TREML1 | 536 |
| 1; ENSG00000161911 | | |
| ENSG00000178199, Q6ZRW2_HUMAN; zinc finger | ZC3H12D | 537 |
| CCCH-type containing 12D | | |
| AIM1; absent in melanoma1; ENSG00000112297 | AIM1 | 538 |
| NKG2D ligand 4 precursor (NKG2D ligand 4) (NKG2DL4) | RAET1E | 539 |
| (N2DL-4) (Retinoic acid early transcript 1E) (Lymphocyte | | |
| effector toxicity activation ligand) (RAE-1-like transcript 4) | | |
| (RL-4), ENSG00000164520, RAET1E | | |
| Disheveled associated activator of morphogenesis 2, | DAAM2 | 540 |
| ENSG00000146122, DAAM2 | | |
| RP11-535K1.1 (Vega gene ID); Putative Processed transcript; | RP11-535K1.1 | 541 |
| OTTHUMG00000014660 | (Vega gene ID) | |
| OTTHUMG00000015679; Novel Protein coding; RP3- | RP3-509I19.3 (Vega | 542 |
| 509I19.3 | gene ID) | |
| RP11-503C24.1 (Vega gene ID); Putative Processed | RP11-503C24.1 | 543 |
| transcript; OTTHUMG00000016040 | (Vega gene ID) | |
| GABRR2; gamma-aminobutyric acid (GABA) receptor, rho | GABRR2 | 544 |
| 2; ENSG00000111886 | | |
| ANKRD6; ankyrin repeat domain 6; ENSG00000135299 | ANKRD6 | 545 |
| TXLNB; taxilin beta; ENSG00000164440 | TXLNB | 546 |
| TXLNB; taxilin beta; ENSG00000164440 | TXLNB | 547 |
| RP5-899B16.2 (Vega gene ID); Putative Processed transcript; | RP5-899B16.2 | 548 |
| OTTHUMG00000015698 | (Vega gene ID) | |
| Probable G-protein coupled receptor 116 precursor, | GPR116 | 549 |
| ENSG00000069122, GPR116 | | |
| RP11-146I2.1 (Vega gene ID); Novel Processed transcript; | RP11-146I2.1 (Vega | 550 |
| OTTHUMG00000014290 | gene ID) | |
| GPR115; G protein-coupled receptor 115; | GPR115 | 551 |
| ENSG00000153294 | | |
| GPR126; G protein-coupled receptor 126; | GPR126 | 552 |
| ENSG00000112414 embryonic marker | | |
| RP1-60O19.1 (Vega gene ID); Known Processed transcript; | RP1-60O19.1 (Vega | 553 |
| OTTHUMG00000015305 | gene ID) | |
| OTTHUMG00000015313, RP1-47M23.1 SCML4 sex comb | SCML4 | 554 |
| on midleg-like 4 (Drosophila) [Homo sapiens] | | |
| OTTHUMG00006004170, TPX1testis specific protein 1 | CRISP2 | 555 |
| (probe H4-1 p3-1) | | |
| OTTHUMG00000014829, RP11-397G17.1, novel processed | RP11-397G17.1 | 556 |
| transcript. | (Vega gene ID) | |
| OTTHUMG00000015337RP11-487F23.3 hypothetical | RP11-487F23.3 | 557 |
| LOC389422 | (Vega gene ID) | |
| Nesprin-1 (Nuclear envelope spectrin repeat protein 1) | SYNE1 | 558 |
| (Synaptic nuclear envelope protein 1) (Syne-1) (Myocyte | | |
| nuclear envelope protein 1) (Myne-1) (Enaptin), | | |
| ENSG00000131018, SYNE1 | | |
| Nesprin-1 (Nuclear envelope spectrin repeat protein 1) | SYNE1 | 559 |
| (Synaptic nuclear envelope protein 1) (Syne-1) (Myocyte | | |
| nuclear envelope protein 1) (Myne-1) (Enaptin), | | |
| ENSG00000131018, SYNE1 | | |
| RP11-398K22.4 (Vega gene ID); Putative Processed | RP11-398K22.4 | 560 |
| transcript; OTTHUMG00000015024 | (Vega gene ID) | |
| MyoD family inhibitor (Myogenic repressor I-mf), | MDFI | 561 |
| ENSG00000112559, MDFI | | |
| OTTHUMG00000014691, putative processed transcript, | RP11-533O20.2 | 562 |
| RP11-533O20.2 | (Vega gene ID) | |
| RP3-398D13.4 (Vega gene ID); OTTHUMG00000014188 | RP3-398D13.4 | 563 |
| | (Vega gene ID); | |
| RP3-429O6.1 (Vega gene ID); Putative Processed transcript; | RP3-429O6.1 (Vega | 564 |
| OTTHUMG00000014195 | gene ID) | |
| MOG; myelin oligodendrocyte glycoprotein; | MOG | 565 |
| ENSG00000137345 | | |
| RP3-495K2.2 (Vega gene ID); Putative Processed transcript; | RP3-495K2.2 (Vega | 566 |
| OTTHUMG00000016052 | gene ID) | |
| RP11-417E7.1 (Vega gene ID); Putative Processed transcript; | RP11-417E7.1 (Vega | 567 |
| OTTHUMG00000016054 | gene ID) | |
| yrosine-protein kinase-like 7 precursor (Colon carcinoma | PTK7 | 568 |
| kinase 4) (CCK-4)., ENSG00000112655, PTK7 | | |
| RP11-174C7.4 (Vega gene ID) | RP11-174C7.4 | 569 |
| | (Vega gene ID) | |
| cytidine monophosphate-N-acetylneuraminic acid | CMAH | 570 |
| hydroxylase (CMP-N-acetylneuraminate monooxygenase); | | |
| CMAH | | |
| PKHD1; polycystic kidney and hepatic disease 1 (autosomal | PKHD1 | 571 |
| recessive); ENSG00000170927 | | |
| RP3-471C18.2 (Vega gene ID); Novel Processed transcript; | RP3-471C18.2 | 572 |
| OTTHUMG00000014332 | (Vega gene ID) | |
| RP11-204E9.1 (Vega gene ID); Putative Processed transcript; | RP11-204E9.1 (Vega | 573 |
| OTTHUMG00000014342 | gene ID) | |
| glutathione peroxidase 5, OTTHUMG00000016307, GPX5 | GPX5 | 574 |
| RP11-411K7.1 (Vega gene ID); Putative Processed transcript; | RP11-411K7.1 | 575 |
| OTTHUMG00000014887 | (Vega gene ID) | |
| skin marker, Glutamate receptor, ionotropic kainate 2 | GRIK2 | 576 |
| precursor (Glutamate receptor 6) (GluR-6) (GluR6) | | |
| (Excitatory amino acid receptor 4) (EAA4) | | |
| C6orf142; chromosome 6 open reading frame 142; | C6orf142 | 577 |
| ENSG00000146147 | | |
| HDGFL1; hepatoma derived growth factor-like 1; | HDGFL1 | 578 |
| ENSG00000112273 | | |
| forkhead box C1, OTTHUMG00000016182, FOXC1 | FOXC1 | 579 |
| C6orf188; chromosome 6 open reading frame 188; | C6orf188 | 580 |
| ENSG00000178033 | | |
| ME1; malic enzyme 1, NADP(+)-dependent, cytosolic; | ME1 | 581 |
| ENSG00000065833 | | |
| SLC22A1; solute carrier family 22 (organic cation | SLC22A1 | 582 |
| transporter), member 1 | | |
| RP11-235G24.1 (Vega gene ID) | RP11-235G24.1 | 583 |
| | (Vega gene ID) | |
| T-box 18; TBX18 | TBX18 | 584 |
| CTA-31J9.2, putative processed transcript, | CTA-31J9.2 (Vega | 585 |
| OTTHUMG00000015619 | gene ID) | |
| RP1-32B1.4 (Vega gene ID); Putative Processed transcript | RP1-32B1.4 (Vega | 586 |
| OTTHUMG00000015628 | gene ID) | |
| OTTHUMG00000014223, RP11-203H2.2, novel processed | RP11-203H2.2 | 587 |
| treanscript | (Vega gene ID) | |
| OTTHUMG00000014737, C6orf154 and Name: chromosome | C6orf154 | 588 |
| 6 open reading frame 154; RP3-337H4.2 | | |
| transcription factor AP-2 alpha, OTTHUMG00000014235, | TFAP2A | 589 |
| TFAP2A | | |
| IL20RA; interleukin 20 receptor, alpha; ENSG00000016402 | IL20RA | 590 |
| KAAG1; kidney associated antigen 1; ENSG00000146049 | KAAG1 | 591 |
| TGM3; transglutaminase 3 (E polypeptide, protein-glutamine- | TGM3 | 592 |
| gamma-glutamyltransferase); ENSG00000125780 | | |
| RASSF2; Ras association (RalGDS/AF-6) domain family 2; | RASSF2 | 593 |
| ENSG00000101265 | | |
| no gene associated | | 594 |
| no gene associated | | 595 |
| no gene associated | | 596 |
| no gene associated | | 597 |
| no gene associated | | 598 |
| no gene associated | | 599 |
| no gene associated | | 600 |
| no gene associated | | 601 |
| no gene associated | | 602 |
| no gene associated | | 603 |
| no gene associated | | 604 |
| RP4-697P8.2 (Vega gene ID); Putative Processed transcript; | RP4-697P8.2 (Vega | 605 |
| OTTHUMG00000031879 | gene ID) | |
| no gene associated | | 606 |
| OTTHUMG00000031883, | RP4-734C18.1 | 607 |
| RP4-734C18.1, putative processed transcript | (Vega gene ID) | |
| no gene associated | | 608 |
| no gene associated | | 609 |
| no gene associated | | 610 |
| no gene associated | | 611 |
| no gene associated | | 612 |
| Ras and Rab interactor 2, | RIN2 | 613 |
| OTTHUMG00000031996, RIN2 | | |
| no gene associated | | 614 |
| no gene associated | | 615 |
| no gene associated | | 616 |
| no gene associated | | 617 |
| no gene associated | | 618 |
| no gene associated | | 619 |
| no gene associated | | 620 |
| no gene associated | | 621 |
| no gene associated | | 622 |
| no gene associated | | 623 |
| C20orf112; chromosome 20 open reading frame 112; | C20orf112 | 624 |
| OTTHUMG00000032219 | | |
| FER1L4; fer-1-like 4 (C. Elegans); OTTHUMG00000032354 | FER1L4 | 625 |
| no gene associated | | 626 |
| no gene associated | | 627 |
| Protein C20orf102 precursor, ENSG00000132821, | | 628 |
| CT102_HUMAN | | |
| no gene associated | | 629 |
| no gene associated | | 630 |
| no gene associated | | 631 |
| no gene associated | | 632 |
| no gene associated - Nearest transcript CDH22 (~18 kb | | 633 |
| upstream) | | |
| no gene associated | | 634 |
| no gene associated | | 635 |
| no gene associated | | 636 |
| no gene associated | | 637 |
| no gene associated | | 638 |
| no gene associated | | 639 |
| no gene associated | | 640 |
| ZHX3; zinc fingers and homeoboxes 3; | ZHX3 | 641 |
| OTTHUMG00000032481 | | |
| no gene associated | | 642 |
| CHD6; chromodomain helicase DNA binding protein 6; | CHD6 | 643 |
| ENSG00000124177 | | |
| no gene associated | | 644 |
| PTPRG; protein tyrosine phosphatase, receptor type G; | PTPRG | 645 |
| ENSG00000144724 | | |
| no gene associated | | 646 |
| no gene associated | | 647 |
| no gene associated | | 648 |
| PTPNS1; protein tyrosine phosphatase, non-receptor type | PTPNS1 | 649 |
| substrate 1; ENSG00000198053 | | |
| Q7Z5T1_HUMAN (Predicted UniProt/TrEMBL ID); | Q7Z5T1_HUMAN | 650 |
| KIAA1442 protein; ENSG00000088881 | (Predicted | |
| | UniProt/TrEMBL | |
| | ID) | |
| NP_689717.2 (RefSeq peptide ID); ENSG00000171984 | NP_689717.2 | 651 |
| | (RefSeq peptide ID) | |
| ENSG00000149346, NP_001009608.1, hypothetical protein | C20orf94 | 652 |
| LOC128710, chromosome 20 open reading frame 94 | | |
| C20orf82; chromosome 20 open reading frame 82; | C20orf82 | 653 |
| ENSG00000101230 | | |
| C20orf23; chromosome 20 open reading frame 23; | C20orf23 | 654 |
| ENSG00000089177; embryonic marker | | |
| PCSK2; proprotein convertase subtilisin/kexin type 2; | PCSK2 | 655 |
| ENSG00000125851 | | |
| PCSK2; proprotein convertase subtilisin/kexin type 2; | PCSK2 | 656 |
| ENSG00000125851 | | |
| solute carrier family 24 (sodiumVpotassiumVcalcium | SLC24A3 | 657 |
| exchanger), member 3, OTTHUMG00000031993, | | |
| SLC24A3 | | |
| solute carrier family 24 (sodiumVpotassiumVcalcium | SLC24A3 | 658 |
| exchanger), member 3, OTTHUMG00000031993, | | |
| SLC24A3 | | |
| ENSG00000089101, CT026_HUMAN | C20orf26 | 659 |
| ENSG00000089101, CT026_HUMAN | C20orf26 | 660 |
| C20orf74 protein, ENSG00000188559, Q9ULE8_HUMAN | Q9ULE8_HUMAN | 661 |
| | (Predicted | |
| | UniProt/TrEMBL | |
| | ID) | |
| C20orf74 protein, ENSG00000188559, Q9ULE8_HUMAN | Q9ULE8_HUMAN | 662 |
| | (Predicted | |
| | UniProt/TrEMBL | |
| | ID) | |
| C20orf74 protein, ENSG00000188559, Q9ULE8_HUMAN | Q9ULE8_HUMAN | 663 |
| | (Predicted | |
| | UniProt/TrEMBL | |
| | ID) | |
| PLAGL2; pleiomorphic adenoma gene-like 2; | PLAGL2 | 664 |
| ENSG00000126003 | | |
| GGTL3; gamma-glutamyltransferase-like 3; | GGTL3 | 665 |
| ENSG00000131067 | | |
| MYH7B; myosin, heavy polypeptide 7B, cardiac muscle, | MYH7B | 666 |
| beta; ENSG00000078814 | | |
| TRPC4AP; transient receptor potential cation channel, | TRPC4AP | 667 |
| subfamily C, member 4 associated protein; | | |
| ENSG00000100991 | | |
| EPB41L1; erythrocyte membrane protein band 4.1-like 1; | EPB41L1 | 668 |
| ENSG00000088367 | | |
| C20orf117; chromosome 20 open reading frame 117; | C20orf117 | 669 |
| OTTHUMG00000032395 | | |
| PTPRT; protein tyrosine phosphatase, receptor type, T; | PTPRT | 670 |
| ENSG00000196090 | | |
| PTPRT; protein tyrosine phosphatase, receptor type, T; | PTPRT | 671 |
| ENSG00000196090 | | |
| PTPRT; protein tyrosine phosphatase, receptor type, T; | PTPRT | 672 |
| ENSG00000196090 | | |
| PTPRT; protein tyrosine phosphatase, receptor type, T; | PTPRT | 673 |
| ENSG00000196090 | | |
| PTPRT; protein tyrosine phosphatase, receptor type, T; | PTPRT | 674 |
| ENSG00000196090 | | |
| SDC4; syndecan 4 (amphiglycan, ryudocan); | SDC4 | 675 |
| ENSG00000124145 | | |
| SDC4; syndecan 4 (amphiglycan, ryudocan); | SDC4 | 676 |
| ENSG00000124145 | | |
| cadherin-like 22, OTTHUMG00000033073, CDH22 | CDH22 | 677 |
| EYA2; eyes absent homolog 2 (Drosophila); | EYA2 | 678 |
| ENSG00000064655 | | |
| SULF2; sulfatase 2; ENSG00000196562 | SULF2 | 679 |
| KCNB1; potassium voltage-gated channel, Shab-related | KCNB1 | 680 |
| subfamily, member 1; ENSG00000158445 | | |
| Breast carcinoma amplified sequence 4, | BCAS4 | 681 |
| ENSG00000124243, | | |
| BCAS4 | | |
| nuclear factor of activated T-cells, cytoplasmic, calcineurin- | NFATC2 | 682 |
| dependent 2, OTTHUMG00000032747, NFATC2 | | |
| Nuclear factor of activated T-cells, cytoplasmic 2 (T cell | NFATC2 | 683 |
| transcription factor NFAT1) (NFAT pre-existing subunit) | | |
| (NF-ATp), ENSG00000101096, NFATC2 | | |
| Bone morphogenetic protein 7 precursor (BMP-7) | BMP7 | 684 |
| (Osteogenic protein 1) (OP-1) (Eptotermin alfa), | | |
| ENSG00000101144, BMP7 | | |
| transmembrane, prostate androgen induced RNA, | TMEPAI | 685 |
| OTTHUMG00000032831, TMEPAI | | |
| ENSG00000176659, NP_775915.1 | NP_775915.1 | 686 |
| | (RefSeq peptide ID) | |
| CDH4; cadherin 4, type 1, R-cadherin (retinal); | CDH4 | 687 |
| ENSG00000179242 | | |
| NP_001002034.1 (RefSeq peptide ID); ENSG00000177096 | NP_001002034.1 | 688 |
| | (RefSeq peptide ID) | |
| NP_612444.1 (RefSeq peptide ID); ENSG00000133477 | NP_612444.1 | 689 |
| | (RefSeq peptide ID) | |
| no gene associated | | 690 |
| OTTHUMG00000030780, CTA-373H7.4, novel pseudogene | CTA-373H7.4 (Vega | 691 |
| | gene ID) | |
| no gene associated | | 692 |
| Cat eye syndrome critical region protein 1 precursor, | CECR1 | 693 |
| ENSG00000093072, CECR1 | | |
| IGLC1; immunoglobulin lambda constant 1 (Mcg marker); | IGLC1 | 694 |
| ENSG00000100208 | | |
| OTTHUMG00000030521, AC000095.4 putative processed | AC000095.4 (Vega | 695 |
| transcript; | gene ID) | |
| Uroplakin-3A precursor (Uroplakin III) (UPIII)., | UPK3A | 696 |
| ENSG00000100373, UPK3A | | |
| Sp1 site_no gene associated | | 697 |
| USP18; ubiquitin specific peptidase 18; | USP18 | 698 |
| OTTHUMG00000030949 | | |
| BCR; breakpoint cluster region; ENSG00000186716 | BCR | 699 |
| TBC1D10A; TBC1 domain family, member 10A; | TBC1D10A | 700 |
| ENSG00000099992 | | |
| signal peptide-CUB domian-EGF-related 1, | SCUBE1 | 701 |
| ENSG00000159307, SCUBE1 | | |
| MAPK8IP2; mitogen-activated protein kinase 8 interacting | MAPK8IP2 | 702 |
| protein 2; ENSG00000008735 | | |
| ENSG00000192797, miRNA | Not Available | 703 |
| RPL3; ribosomal protein L3; ENSG00000100316 | RPL3 | 704 |
| RPL3; ribosomal protein L3; ENSG00000100316 | RPL3 | 705 |
| RP4-695O20_B.9 (Vega gene ID); Putative Processed | RP4-695O20_B.9 | 706 |
| transcript; OTTHUMG00000030111 | (Vega gene ID) | |
| No associated gene | | 707 |
| MN1; meningioma (disrupted in balanced translocation) 1; | MN1 | 708 |
| ENSG00000169184 | | |
| no gene associated | | 709 |
| RTDR1; rhabdoid tumor deletion region gene 1; | RTDR1 | 710 |
| ENSG00000100218 | | |
| RPL3; ribosomal protein L3; ENSG00000100316 | RPL3 | 711 |
| embryonic marker, GRB2-related adaptor protein 2, | GRAP2 | 712 |
| OTTHUMG00000030700, GRAP2 | | |
| Serine/threonine-protein kinase 19 (EC 2.7.1.37) (RP1 | STK19 | 713 |
| protein) (G11 protein). | | |
| Transcription factor 19 (Transcription factor SC1). | TCF19_HUMAN | 714 |
| Pannexin-2 | PANX2 | 715 |
| | CTA-243E7.3 | 716 |
| signal peptide-CUB domian-EGF-related 1 | SCUBE1 | 717 |
| Reticulon 4 receptor precursor (Nogo receptor) (NgR) (Nogo- | RTN4R | 718 |
| 66 receptor) | | |
| Arylsulfatase A precursor (EC 3.1.6.8) (ASA) (Cerebroside- | ARSA | 719 |
| sulfatase) [Contains: Arylsulfatase A component B; | | |
| Arylsulfatase A component C] | | |
| glycoprotein Ib (platelet), beta polypeptide | GP1BB | 720 |
| | No gene associated | 721 |
| | No gene associated | 722 |
| Mitochondrial glutamate carrier 2 (Glutamate/H(+) symporter | SLC25A18 | 723 |
| 2) (Solute carrier family 25 member 18, ENSG00000182902, | | |
| SLC25A18 | | |
| Thioredoxin reductase 2, mitochondrial precursor (EC | TXNRD2 | 724 |
| 1.8.1.9) (TR3) (TR-beta) (Selenoprotein Z) (SelZ) | | |
| Somatostatin receptor type 3 (SS3R) (SSR-28) | SSTR3 | 725 |
| | RP11-191L9.1 | 726 |
| No description-pseudogene | AP000357.3 | 727 |
| Cat eye syndrome critical region protein 1 precursor | CECR1 | 728 |
| | No gene associated | 729 |
| Membrane protein MLC1 | MLC1 | 730 |
| BAI1-associated protein 2-like 2 | BAIAP2L2 | 731 |
| No description | NP_056185.1 | 732 |
| No description | RP4-695O20_B.9 | 733 |
| OTTHUMG00000030167, CTA-243E7.3 | CTA-243E7.3 (Vega | 734 |
| | gene ID) | |
| novel transcript | XXbac-B444P24.7 | 735 |
| | LL22NC03-121E8.1- | 736 |
| | 001 | |
| No description | Q6ZN90_HUMAN | 737 |
| NFAT activation molecule 1 precursor (Calcineurin/NFAT- | NFAM1 | 738 |
| activating ITAM-containing protein) (NFAT activating | | |
| protein with ITAM motif 1). | | |
| immunoglobulin lambda constant 2 | IGLC2 | 739 |
| immunoglobulin lambda constant 2 | IGLC2 | 740 |
| OTTHUMG00000030870, CTA-503F6.1 | CTA-503F6.1 | 741 |
| | (Vega_gene ID) | |
| Lactosylceramide 4-alpha-galactosyltransferase (EC | A4GALT | 742 |
| 2.4.1.228) | | |
| | RP11-191L9.3 | 743 |
| Cold shock domain protein C2 (RNA-binding protein PIPPin) | CSDC2_HUMAN | 744 |
| GAS2-like protein 1 (Growth arrest-specific 2-like 1) (GAS2- | GAS2L1 | 745 |
| related protein on chromosome 22) (GAR22 protein), | | |
| ENSG00000185340, GAS2L1 | | |
| BAI1-associated protein 2-like 2 | BAIAP2L2 | 746 |
| | NP_997360.1 | 747 |
| OTTHUMG00000030991, LL22NC03-75B3.6 | LL22NC03-75B3.6 | 748 |
| | (Vega gene ID) | |
| Reticulon 4 receptor precursor (Nogo receptor) (NgR) (Nogo- | RTN4R | 749 |
| 66 receptor) | | |
| Smoothelin | SMTN | 750 |
| solute carrier family 35, member E4 | SLC35E4 | 751 |
| Protein C22orf13 (Protein LLN4) | CV013_HUMAN | 752 |
| | No gene associated | 753 |
| Histone | HIST1H3A | 754 |
| Gamma-aminobutyric-acid receptor rho-1 subunit precursor | GABRR1 | 755 |
| (GABA(A) receptor). | | |
| OTTHUMG00000015693, RP11-12A2.3 | RP11-12A2.3 | 756 |
| | (Vega_gene ID) | |
| | RP5-899B16.1 | 757 |
| | RP11-146I2.2 | 758 |
| | NP_060483.2 | 759 |
| Forkhead box protein O3A, ENSG00000118689, FOXO3A | FOXO3A | 760 |
| nuclear receptor coactivator 7 | NCOA7 | 761 |
| | RP11-554D15.1 | 762 |
| chromosome 6 open reading frame 190 | C6orf190 | 763 |
| phosphatase and actin regulator 2 | PHACTR2 | 764 |
| High mobility group protein HMG-I/HMG-Y, HMG-I(Y), | HMGA1 | 765 |
| High mobility group AT-hook 1, High mobility group protein | | |
| A1, ENSG00000137309, HMGA1 | | |
| Pantetheinase precursor (EC 3.5.1.—), ENSG00000112299, | VNN1 | 766 |
| VNN1 | | |
| histone H2A | HIST1H2AA | 767 |
| transcription factor AP-2 alpha (activating enhancer binding | TFAP2A | 768 |
| protein 2 alpha) | | |
| N-acetyllactosaminide beta-1,6-N-acetylglucosaminyl- | GCNT2 | 769 |
| transferase (EC 2.4.1.150), ENSG00000111846, GCNT2 | | |
| | No gene associated | 770 |
| | No gene associated | 771 |
| | No gene associated | 772 |
| | No gene associated | 773 |
| | No gene associated | 774 |
| | No gene associated | 775 |
| | No gene associated | 776 |
| | No gene associated | 777 |
| | No gene associated | 778 |
| | No gene associated | 779 |
| | RP11-318C17.1 | 780 |
| | No gene associated | 781 |
| | No gene associated | 782 |
| | No gene associated | 783 |
| | No gene associated | 784 |
| | No gene associated | 785 |
| | No gene associated | 786 |
| novel transcript | RP11-216C10.1 | 787 |
| | No gene associated | 788 |
| | No gene associated | 789 |
| | No gene associated | 790 |
| | RP11-410N8.3 | 791 |
| TIMP3; TIMP metallopeptidase inhibitor 3 (Sorsby fundus | TIMP3 | 792 |
| dystrophy, pseudoinflammatory); ENSG00000100234 | | |
| | No gene associated | 793 |
| | No gene associated | 794 |
| | No gene associated | 795 |
| | No gene associated | 796 |
| | No gene associated | 797 |
| | No gene associated | 798 |
| | No gene associated | 799 |
| | No gene associated | 800 |
| | No gene associated | 801 |
| | No gene associated | 802 |
| | No gene associated | 803 |
| sorting nexin 5 | SNX5 | 804 |
| Probable D-tyrosyl-tRNA(Tyr) deacylase (EC 3.1.—.—) | HARS2 | 805 |
| solute carrier family 24 (sodium/potassium/calcium | SLC24A3 | 806 |
| exchanger) member 3, OTTHUMG00000031993, SLC24A3 | | |
| ENSG00000089101, CT026_HUMAN, C20orf26 | CT026_HUMAN | 807 |
| RNA-binding protein Raly (hnRNP associated with lethal | RALY | 808 |
| yellow homolog) D; ENSG00000125970, RALY | | |
| Protein phosphatase 1 regulatory inhibitor subunit 16B (TGF- | PPP1R16B | 809 |
| beta-inhibited membrane-associated protein) (hTIMAP) | | |
| (CAAX box protein TIMAP) (Ankyrin repeat domain protein | | |
| 4) | | |
| protein tyrosine phosphatase, receptor type, T | PTPRT | 810 |
| protein tyrosine phosphatase, receptor type, T | PTPRT | 811 |
| protein tyrosine phosphatase, receptor type, T | PTPRT | 812 |
| Receptor-type tyrosine-protein phosphatase T precursor (EC | PTPRT | 813 |
| 3.1.3.48) (R-PTP-T) (RPTP-rho) | | |
| cadherin-like 22 | CDH22 | 814 |
| potassium voltage-gated channel, Shab-related subfamily, | KCNB1 | 815 |
| member 1 | | |
| potassium voltage-gated channel, Shab-related subfamily, | KCNB1 | 816 |
| member 1 | | |
| Zinc finger protein SNAI1 (Snail protein homolog) (Sna | SNAI1 | 817 |
| protein) | | |
| Cadherin-4 precursor (Retinal-cadherin) (R-cadherin) (R- | CDH4 | 818 |
| CAD) | | |
| cadherin 4, type 1, R-cadherin (retinal) | CDH4 | 819 |
| Cadherin-4 precursor (Retinal-cadherin) (R-cadherin) (R- | CDH4 | 820 |
| CAD) | | |
| Metalloproteinase inhibitor 3 precursor (TIMP-3) (Tissue | TIMP3 | 821 |
| inhibitor of metalloproteinases-3) (MIG-5 protein). | | |
| Tubulin alpha-8 chain (Alpha-tubulin 8) | TUBA8 | 822 |
| | No gene associated | 823 |
| | No gene associated | 824 |

TABLE 2
Overview of SEQ ID NO of the genes or genomic regions and the
SEQ ID NOs of corresponding bisulfite treated nucleic acids (DNA
upmethylated sense strand, DNA upmethylated antisense strand,
DNA downmethylated sense strand, DNA downmethylated antisense
strand).

| | | | SEQ ID NO: | SEQ ID NO: |
|---|---|---|---|---|
| SEQ ID | SEQ ID NO | SEQ ID NO: | Down- | Down- |
| NO: | Upmethylated | Upmethylated | methylated | methylated |
| Genomic | Sense | Antisense | Sense | Antisense |
| 413 | 1649 | 1650 | 3297 | 3298 |
| 414 | 1651 | 1652 | 3299 | 3300 |
| 415 | 1653 | 1654 | 3301 | 3302 |
| 416 | 1655 | 1656 | 3303 | 3304 |
| 417 | 1657 | 1658 | 3305 | 3306 |
| 418 | 1659 | 1660 | 3307 | 3308 |
| 419 | 1661 | 1662 | 3309 | 3310 |
| 420 | 1663 | 1664 | 3311 | 3312 |
| 421 | 1665 | 1666 | 3313 | 3314 |
| 422 | 1667 | 1668 | 3315 | 3316 |
| 423 | 1669 | 1670 | 3317 | 3318 |
| 424 | 1671 | 1672 | 3319 | 3320 |
| 425 | 1673 | 1674 | 3321 | 3322 |
| 426 | 1675 | 1676 | 3323 | 3324 |
| 427 | 1677 | 1678 | 3325 | 3326 |
| 428 | 1679 | 1680 | 3327 | 3328 |
| 429 | 1681 | 1682 | 3329 | 3330 |
| 430 | 1683 | 1684 | 3331 | 3332 |
| 431 | 1685 | 1686 | 3333 | 3334 |
| 432 | 1687 | 1688 | 3335 | 3336 |
| 433 | 1689 | 1690 | 3337 | 3338 |
| 434 | 1691 | 1692 | 3339 | 3340 |
| 435 | 1693 | 1694 | 3341 | 3342 |
| 436 | 1695 | 1696 | 3343 | 3344 |
| 437 | 1697 | 1698 | 3345 | 3346 |
| 438 | 1699 | 1700 | 3347 | 3348 |
| 439 | 1701 | 1702 | 3349 | 3350 |
| 440 | 1703 | 1704 | 3351 | 3352 |
| 441 | 1705 | 1706 | 3353 | 3354 |
| 442 | 1707 | 1708 | 3355 | 3356 |
| 443 | 1709 | 1710 | 3357 | 3358 |
| 444 | 1711 | 1712 | 3359 | 3360 |
| 445 | 1713 | 1714 | 3361 | 3362 |
| 446 | 1715 | 1716 | 3363 | 3364 |
| 447 | 1717 | 1718 | 3365 | 3366 |
| 448 | 1719 | 1720 | 3367 | 3368 |
| 449 | 1721 | 1722 | 3369 | 3370 |
| 450 | 1723 | 1724 | 3371 | 3372 |
| 451 | 1725 | 1726 | 3373 | 3374 |
| 452 | 1727 | 1728 | 3375 | 3376 |
| 453 | 1729 | 1730 | 3377 | 3378 |
| 454 | 1731 | 1732 | 3379 | 3380 |
| 455 | 1733 | 1734 | 3381 | 3382 |
| 456 | 1735 | 1736 | 3383 | 3384 |
| 457 | 1737 | 1738 | 3385 | 3386 |
| 458 | 1739 | 1740 | 3387 | 3388 |
| 459 | 1741 | 1742 | 3389 | 3390 |
| 460 | 1743 | 1744 | 3391 | 3392 |
| 461 | 1745 | 1746 | 3393 | 3394 |
| 462 | 1747 | 1748 | 3395 | 3396 |
| 463 | 1749 | 1750 | 3397 | 3398 |
| 464 | 1751 | 1752 | 3399 | 3400 |
| 465 | 1753 | 1754 | 3401 | 3402 |
| 466 | 1755 | 1756 | 3403 | 3404 |
| 467 | 1757 | 1758 | 3405 | 3406 |
| 468 | 1759 | 1760 | 3407 | 3408 |
| 469 | 1761 | 1762 | 3409 | 3410 |
| 470 | 1763 | 1764 | 3411 | 3412 |
| 471 | 1765 | 1766 | 3413 | 3414 |
| 472 | 1767 | 1768 | 3415 | 3416 |
| 473 | 1769 | 1770 | 3417 | 3418 |
| 474 | 1771 | 1772 | 3419 | 3420 |
| 475 | 1773 | 1774 | 3421 | 3422 |
| 476 | 1775 | 1776 | 3423 | 3424 |
| 477 | 1777 | 1778 | 3425 | 3426 |
| 478 | 1779 | 1780 | 3427 | 3428 |
| 479 | 1781 | 1782 | 3429 | 3430 |
| 480 | 1783 | 1784 | 3431 | 3432 |
| 481 | 1785 | 1786 | 3433 | 3434 |
| 482 | 1787 | 1788 | 3435 | 3436 |
| 483 | 1789 | 1790 | 3437 | 3438 |
| 484 | 1791 | 1792 | 3439 | 3440 |
| 485 | 1793 | 1794 | 3441 | 3442 |
| 486 | 1795 | 1796 | 3443 | 3444 |
| 487 | 1797 | 1798 | 3445 | 3446 |
| 488 | 1799 | 1800 | 3447 | 3448 |
| 489 | 1801 | 1802 | 3449 | 3450 |
| 490 | 1803 | 1804 | 3451 | 3452 |
| 491 | 1805 | 1806 | 3453 | 3454 |
| 492 | 1807 | 1808 | 3455 | 3456 |
| 493 | 1809 | 1810 | 3457 | 3458 |
| 494 | 1811 | 1812 | 3459 | 3460 |
| 495 | 1813 | 1814 | 3461 | 3462 |
| 496 | 1815 | 1816 | 3463 | 3464 |
| 497 | 1817 | 1818 | 3465 | 3466 |
| 498 | 1819 | 1820 | 3467 | 3468 |
| 499 | 1821 | 1822 | 3469 | 3470 |
| 500 | 1823 | 1824 | 3471 | 3472 |
| 501 | 1825 | 1826 | 3473 | 3474 |
| 502 | 1827 | 1828 | 3475 | 3476 |
| 503 | 1829 | 1830 | 3477 | 3478 |
| 504 | 1831 | 1832 | 3479 | 3480 |
| 505 | 1833 | 1834 | 3481 | 3482 |
| 506 | 1835 | 1836 | 3483 | 3484 |
| 507 | 1837 | 1838 | 3485 | 3486 |
| 508 | 1839 | 1840 | 3487 | 3488 |
| 509 | 1841 | 1842 | 3489 | 3490 |
| 510 | 1843 | 1844 | 3491 | 3492 |
| 511 | 1845 | 1846 | 3493 | 3494 |
| 512 | 1847 | 1848 | 3495 | 3496 |
| 513 | 1849 | 1850 | 3497 | 3498 |
| 514 | 1851 | 1852 | 3499 | 3500 |
| 515 | 1853 | 1854 | 3501 | 3502 |
| 516 | 1855 | 1856 | 3503 | 3504 |
| 517 | 1857 | 1858 | 3505 | 3506 |
| 518 | 1859 | 1860 | 3507 | 3508 |
| 519 | 1861 | 1862 | 3509 | 3510 |
| 520 | 1863 | 1864 | 3511 | 3512 |
| 521 | 1865 | 1866 | 3513 | 3514 |
| 522 | 1867 | 1868 | 3515 | 3516 |
| 523 | 1869 | 1870 | 3517 | 3518 |
| 524 | 1871 | 1872 | 3519 | 3520 |
| 525 | 1873 | 1874 | 3521 | 3522 |
| 526 | 1875 | 1876 | 3523 | 3524 |
| 527 | 1877 | 1878 | 3525 | 3526 |
| 528 | 1879 | 1880 | 3527 | 3528 |
| 529 | 1881 | 1882 | 3529 | 3530 |
| 530 | 1883 | 1884 | 3531 | 3532 |
| 531 | 1885 | 1886 | 3533 | 3534 |
| 532 | 1887 | 1888 | 3535 | 3536 |
| 533 | 1889 | 1890 | 3537 | 3538 |
| 534 | 1891 | 1892 | 3539 | 3540 |
| 535 | 1893 | 1894 | 3541 | 3542 |
| 536 | 1895 | 1896 | 3543 | 3544 |
| 537 | 1897 | 1898 | 3545 | 3546 |
| 538 | 1899 | 1900 | 3547 | 3548 |
| 539 | 1901 | 1902 | 3549 | 3550 |
| 540 | 1903 | 1904 | 3551 | 3552 |
| 541 | 1905 | 1906 | 3553 | 3554 |
| 542 | 1907 | 1908 | 3555 | 3556 |
| 543 | 1909 | 1910 | 3557 | 3558 |
| 544 | 1911 | 1912 | 3559 | 3560 |
| 545 | 1913 | 1914 | 3561 | 3562 |
| 546 | 1915 | 1916 | 3563 | 3564 |
| 547 | 1917 | 1918 | 3565 | 3566 |
| 548 | 1919 | 1920 | 3567 | 3568 |
| 549 | 1921 | 1922 | 3569 | 3570 |
| 550 | 1923 | 1924 | 3571 | 3572 |
| 551 | 1925 | 1926 | 3573 | 3574 |
| 552 | 1927 | 1928 | 3575 | 3576 |
| 553 | 1929 | 1930 | 3577 | 3578 |
| 554 | 1931 | 1932 | 3579 | 3580 |
| 555 | 1933 | 1934 | 3581 | 3582 |
| 556 | 1935 | 1936 | 3583 | 3584 |
| 557 | 1937 | 1938 | 3585 | 3586 |
| 558 | 1939 | 1940 | 3587 | 3588 |
| 559 | 1941 | 1942 | 3589 | 3590 |
| 560 | 1943 | 1944 | 3591 | 3592 |
| 561 | 1945 | 1946 | 3593 | 3594 |
| 562 | 1947 | 1948 | 3595 | 3596 |
| 563 | 1949 | 1950 | 3597 | 3598 |
| 564 | 1951 | 1952 | 3599 | 3600 |
| 565 | 1953 | 1954 | 3601 | 3602 |
| 566 | 1955 | 1956 | 3603 | 3604 |
| 567 | 1957 | 1958 | 3605 | 3606 |
| 568 | 1959 | 1960 | 3607 | 3608 |
| 569 | 1961 | 1962 | 3609 | 3610 |
| 570 | 1963 | 1964 | 3611 | 3612 |
| 571 | 1965 | 1966 | 3613 | 3614 |
| 572 | 1967 | 1968 | 3615 | 3616 |
| 573 | 1969 | 1970 | 3617 | 3618 |
| 574 | 1971 | 1972 | 3619 | 3620 |
| 575 | 1973 | 1974 | 3621 | 3622 |
| 576 | 1975 | 1976 | 3623 | 3624 |
| 577 | 1977 | 1978 | 3625 | 3626 |
| 578 | 1979 | 1980 | 3627 | 3628 |
| 579 | 1981 | 1982 | 3629 | 3630 |
| 580 | 1983 | 1984 | 3631 | 3632 |
| 581 | 1985 | 1986 | 3633 | 3634 |
| 582 | 1987 | 1988 | 3635 | 3636 |
| 583 | 1989 | 1990 | 3637 | 3638 |
| 584 | 1991 | 1992 | 3639 | 3640 |
| 585 | 1993 | 1994 | 3641 | 3642 |
| 586 | 1995 | 1996 | 3643 | 3644 |
| 587 | 1997 | 1998 | 3645 | 3646 |
| 588 | 1999 | 2000 | 3647 | 3648 |
| 589 | 2001 | 2002 | 3649 | 3650 |
| 590 | 2003 | 2004 | 3651 | 3652 |
| 591 | 2005 | 2006 | 3653 | 3654 |
| 592 | 2007 | 2008 | 3655 | 3656 |
| 593 | 2009 | 2010 | 3657 | 3658 |
| 594 | 2011 | 2012 | 3659 | 3660 |
| 595 | 2013 | 2014 | 3661 | 3662 |
| 596 | 2015 | 2016 | 3663 | 3664 |
| 597 | 2017 | 2018 | 3665 | 3666 |
| 598 | 2019 | 2020 | 3667 | 3668 |
| 599 | 2021 | 2022 | 3669 | 3670 |
| 600 | 2023 | 2024 | 3671 | 3672 |
| 601 | 2025 | 2026 | 3673 | 3674 |
| 602 | 2027 | 2028 | 3675 | 3676 |
| 603 | 2029 | 2030 | 3677 | 3678 |
| 604 | 2031 | 2032 | 3679 | 3680 |
| 605 | 2033 | 2034 | 3681 | 3682 |
| 606 | 2035 | 2036 | 3683 | 3684 |
| 607 | 2037 | 2038 | 3685 | 3686 |
| 608 | 2039 | 2040 | 3687 | 3688 |
| 609 | 2041 | 2042 | 3689 | 3690 |
| 610 | 2043 | 2044 | 3691 | 3692 |
| 611 | 2045 | 2046 | 3693 | 3694 |
| 612 | 2047 | 2048 | 3695 | 3696 |
| 613 | 2049 | 2050 | 3697 | 3698 |
| 614 | 2051 | 2052 | 3699 | 3700 |
| 615 | 2053 | 2054 | 3701 | 3702 |
| 616 | 2055 | 2056 | 3703 | 3704 |
| 617 | 2057 | 2058 | 3705 | 3706 |
| 618 | 2059 | 2060 | 3707 | 3708 |
| 619 | 2061 | 2062 | 3709 | 3710 |
| 620 | 2063 | 2064 | 3711 | 3712 |
| 621 | 2065 | 2066 | 3713 | 3714 |
| 622 | 2067 | 2068 | 3715 | 3716 |
| 623 | 2069 | 2070 | 3717 | 3718 |
| 624 | 2071 | 2072 | 3719 | 3720 |
| 625 | 2073 | 2074 | 3721 | 3722 |
| 626 | 2075 | 2076 | 3723 | 3724 |
| 627 | 2077 | 2078 | 3725 | 3726 |
| 628 | 2079 | 2080 | 3727 | 3728 |
| 629 | 2081 | 2082 | 3729 | 3730 |
| 630 | 2083 | 2084 | 3731 | 3732 |
| 631 | 2085 | 2086 | 3733 | 3734 |
| 632 | 2087 | 2088 | 3735 | 3736 |
| 633 | 2089 | 2090 | 3737 | 3738 |
| 634 | 2091 | 2092 | 3739 | 3740 |
| 635 | 2093 | 2094 | 3741 | 3742 |
| 636 | 2095 | 2096 | 3743 | 3744 |
| 637 | 2097 | 2098 | 3745 | 3746 |
| 638 | 2099 | 2100 | 3747 | 3748 |
| 639 | 2101 | 2102 | 3749 | 3750 |
| 640 | 2103 | 2104 | 3751 | 3752 |
| 641 | 2105 | 2106 | 3753 | 3754 |
| 642 | 2107 | 2108 | 3755 | 3756 |
| 643 | 2109 | 2110 | 3757 | 3758 |
| 644 | 2111 | 2112 | 3759 | 3760 |
| 645 | 2113 | 2114 | 3761 | 3762 |
| 646 | 2115 | 2116 | 3763 | 3764 |
| 647 | 2117 | 2118 | 3765 | 3766 |
| 648 | 2119 | 2120 | 3767 | 3768 |
| 649 | 2121 | 2122 | 3769 | 3770 |
| 650 | 2123 | 2124 | 3771 | 3772 |
| 651 | 2125 | 2126 | 3773 | 3774 |
| 652 | 2127 | 2128 | 3775 | 3776 |
| 653 | 2129 | 2130 | 3777 | 3778 |
| 654 | 2131 | 2132 | 3779 | 3780 |
| 655 | 2133 | 2134 | 3781 | 3782 |
| 656 | 2135 | 2136 | 3783 | 3784 |
| 657 | 2137 | 2138 | 3785 | 3786 |
| 658 | 2139 | 2140 | 3787 | 3788 |
| 659 | 2141 | 2142 | 3789 | 3790 |
| 660 | 2143 | 2144 | 3791 | 3792 |
| 661 | 2145 | 2146 | 3793 | 3794 |
| 662 | 2147 | 2148 | 3795 | 3796 |
| 663 | 2149 | 2150 | 3797 | 3798 |
| 664 | 2151 | 2152 | 3799 | 3800 |
| 665 | 2153 | 2154 | 3801 | 3802 |
| 666 | 2155 | 2156 | 3803 | 3804 |
| 667 | 2157 | 2158 | 3805 | 3806 |
| 668 | 2159 | 2160 | 3807 | 3808 |
| 669 | 2161 | 2162 | 3809 | 3810 |
| 670 | 2163 | 2164 | 3811 | 3812 |
| 671 | 2165 | 2166 | 3813 | 3814 |
| 672 | 2167 | 2168 | 3815 | 3816 |
| 673 | 2169 | 2170 | 3817 | 3818 |
| 674 | 2171 | 2172 | 3819 | 3820 |
| 675 | 2173 | 2174 | 3821 | 3822 |
| 676 | 2175 | 2176 | 3823 | 3824 |
| 677 | 2177 | 2178 | 3825 | 3826 |
| 678 | 2179 | 2180 | 3827 | 3828 |
| 679 | 2181 | 2182 | 3829 | 3830 |
| 680 | 2183 | 2184 | 3831 | 3832 |
| 681 | 2185 | 2186 | 3833 | 3834 |
| 682 | 2187 | 2188 | 3835 | 3836 |
| 683 | 2189 | 2190 | 3837 | 3838 |
| 684 | 2191 | 2192 | 3839 | 3840 |
| 685 | 2193 | 2194 | 3841 | 3842 |
| 686 | 2195 | 2196 | 3843 | 3844 |
| 687 | 2197 | 2198 | 3845 | 3846 |
| 688 | 2199 | 2200 | 3847 | 3848 |
| 689 | 2201 | 2202 | 3849 | 3850 |
| 690 | 2203 | 2204 | 3851 | 3852 |
| 691 | 2205 | 2206 | 3853 | 3854 |
| 692 | 2207 | 2208 | 3855 | 3856 |
| 693 | 2209 | 2210 | 3857 | 3858 |
| 694 | 2211 | 2212 | 3859 | 3860 |
| 695 | 2213 | 2214 | 3861 | 3862 |
| 696 | 2215 | 2216 | 3863 | 3864 |
| 697 | 2217 | 2218 | 3865 | 3866 |
| 698 | 2219 | 2220 | 3867 | 3868 |
| 699 | 2221 | 2222 | 3869 | 3870 |
| 700 | 2223 | 2224 | 3871 | 3872 |
| 701 | 2225 | 2226 | 3873 | 3874 |
| 702 | 2227 | 2228 | 3875 | 3876 |
| 703 | 2229 | 2230 | 3877 | 3878 |
| 704 | 2231 | 2232 | 3879 | 3880 |
| 705 | 2233 | 2234 | 3881 | 3882 |
| 706 | 2235 | 2236 | 3883 | 3884 |
| 707 | 2237 | 2238 | 3885 | 3886 |
| 708 | 2239 | 2240 | 3887 | 3888 |
| 709 | 2241 | 2242 | 3889 | 3890 |
| 710 | 2243 | 2244 | 3891 | 3892 |
| 711 | 2245 | 2246 | 3893 | 3894 |
| 712 | 2247 | 2248 | 3895 | 3896 |
| 713 | 2249 | 2250 | 3897 | 3898 |
| 714 | 2251 | 2252 | 3899 | 3900 |
| 715 | 2253 | 2254 | 3901 | 3902 |
| 716 | 2255 | 2256 | 3903 | 3904 |
| 717 | 2257 | 2258 | 3905 | 3906 |
| 718 | 2259 | 2260 | 3907 | 3908 |
| 719 | 2261 | 2262 | 3909 | 3910 |
| 720 | 2263 | 2264 | 3911 | 3912 |
| 721 | 2265 | 2266 | 3913 | 3914 |
| 722 | 2267 | 2268 | 3915 | 3916 |
| 723 | 2269 | 2270 | 3917 | 3918 |
| 724 | 2271 | 2272 | 3919 | 3920 |
| 725 | 2273 | 2274 | 3921 | 3922 |
| 726 | 2275 | 2276 | 3923 | 3924 |
| 727 | 2277 | 2278 | 3925 | 3926 |
| 728 | 2279 | 2280 | 3927 | 3928 |
| 729 | 2281 | 2282 | 3929 | 3930 |
| 730 | 2283 | 2284 | 3931 | 3932 |
| 731 | 2285 | 2286 | 3933 | 3934 |
| 732 | 2287 | 2288 | 3935 | 3936 |
| 733 | 2289 | 2290 | 3937 | 3938 |
| 734 | 2291 | 2292 | 3939 | 3940 |
| 735 | 2293 | 2294 | 3941 | 3942 |
| 736 | 2295 | 2296 | 3943 | 3944 |
| 737 | 2297 | 2298 | 3945 | 3946 |
| 738 | 2299 | 2300 | 3947 | 3948 |
| 739 | 2301 | 2302 | 3949 | 3950 |
| 740 | 2303 | 2304 | 3951 | 3952 |
| 741 | 2305 | 2306 | 3953 | 3954 |
| 742 | 2307 | 2308 | 3955 | 3956 |
| 743 | 2309 | 2310 | 3957 | 3958 |
| 744 | 2311 | 2312 | 3959 | 3960 |
| 745 | 2313 | 2314 | 3961 | 3962 |
| 746 | 2315 | 2316 | 3963 | 3964 |
| 747 | 2317 | 2318 | 3965 | 3966 |
| 748 | 2319 | 2320 | 3967 | 3968 |
| 749 | 2321 | 2322 | 3969 | 3970 |
| 750 | 2323 | 2324 | 3971 | 3972 |
| 751 | 2325 | 2326 | 3973 | 3974 |
| 752 | 2327 | 2328 | 3975 | 3976 |
| 753 | 2329 | 2330 | 3977 | 3978 |
| 754 | 2331 | 2332 | 3979 | 3980 |
| 755 | 2333 | 2334 | 3981 | 3982 |
| 756 | 2335 | 2336 | 3983 | 3984 |
| 757 | 2337 | 2338 | 3985 | 3986 |
| 758 | 2339 | 2340 | 3987 | 3988 |
| 759 | 2341 | 2342 | 3989 | 3990 |
| 760 | 2343 | 2344 | 3991 | 3992 |
| 761 | 2345 | 2346 | 3993 | 3994 |
| 762 | 2347 | 2348 | 3995 | 3996 |
| 763 | 2349 | 2350 | 3997 | 3998 |
| 764 | 2351 | 2352 | 3999 | 4000 |
| 765 | 2353 | 2354 | 4001 | 4002 |
| 766 | 2355 | 2356 | 4003 | 4004 |
| 767 | 2357 | 2358 | 4005 | 4006 |
| 768 | 2359 | 2360 | 4007 | 4008 |
| 769 | 2361 | 2362 | 4009 | 4010 |
| 770 | 2363 | 2364 | 4011 | 4012 |
| 771 | 2365 | 2366 | 4013 | 4014 |
| 772 | 2367 | 2368 | 4015 | 4016 |
| 773 | 2369 | 2370 | 4017 | 4018 |
| 774 | 2371 | 2372 | 4019 | 4020 |
| 775 | 2373 | 2374 | 4021 | 4022 |
| 776 | 2375 | 2376 | 4023 | 4024 |
| 777 | 2377 | 2378 | 4025 | 4026 |
| 778 | 2379 | 2380 | 4027 | 4028 |
| 779 | 2381 | 2382 | 4029 | 4030 |
| 780 | 2383 | 2384 | 4031 | 4032 |
| 781 | 2385 | 2386 | 4033 | 4034 |
| 782 | 2387 | 2388 | 4035 | 4036 |
| 783 | 2389 | 2390 | 4037 | 4038 |
| 784 | 2391 | 2392 | 4039 | 4040 |
| 785 | 2393 | 2394 | 4041 | 4042 |
| 786 | 2395 | 2396 | 4043 | 4044 |
| 787 | 2397 | 2398 | 4045 | 4046 |
| 788 | 2399 | 2400 | 4047 | 4048 |
| 789 | 2401 | 2402 | 4049 | 4050 |
| 790 | 2403 | 2404 | 4051 | 4052 |
| 791 | 2405 | 2406 | 4053 | 4054 |
| 792 | 2407 | 2408 | 4055 | 4056 |
| 793 | 2409 | 2410 | 4057 | 4058 |
| 794 | 2411 | 2412 | 4059 | 4060 |
| 795 | 2413 | 2414 | 4061 | 4062 |
| 796 | 2415 | 2416 | 4063 | 4064 |
| 797 | 2417 | 2418 | 4065 | 4066 |
| 798 | 2419 | 2420 | 4067 | 4068 |
| 799 | 2421 | 2422 | 4069 | 4070 |
| 800 | 2423 | 2424 | 4071 | 4072 |
| 801 | 2425 | 2426 | 4073 | 4074 |
| 802 | 2427 | 2428 | 4075 | 4076 |
| 803 | 2429 | 2430 | 4077 | 4078 |
| 804 | 2431 | 2432 | 4079 | 4080 |
| 805 | 2433 | 2434 | 4081 | 4082 |
| 806 | 2435 | 2436 | 4083 | 4084 |
| 807 | 2437 | 2438 | 4085 | 4086 |
| 808 | 2439 | 2440 | 4087 | 4088 |
| 809 | 2441 | 2442 | 4089 | 4090 |
| 810 | 2443 | 2444 | 4091 | 4092 |
| 811 | 2445 | 2446 | 4093 | 4094 |
| 812 | 2447 | 2448 | 4095 | 4096 |
| 813 | 2449 | 2450 | 4097 | 4098 |
| 814 | 2451 | 2452 | 4099 | 4100 |
| 815 | 2453 | 2454 | 4101 | 4102 |
| 816 | 2455 | 2456 | 4103 | 4104 |
| 817 | 2457 | 2458 | 4105 | 4106 |
| 818 | 2459 | 2460 | 4107 | 4108 |
| 819 | 2461 | 2462 | 4109 | 4110 |
| 820 | 2463 | 2464 | 4111 | 4112 |
| 821 | 2465 | 2466 | 4113 | 4114 |
| 822 | 2467 | 2468 | 4115 | 4116 |
| 823 | 2469 | 2470 | 4117 | 4118 |
| 824 | 2471 | 2472 | 4119 | 4120 |

TABLE 3
Overview of SEQ ID NO of amplificates derived from the genes or
genomic regions and the SEQ ID NOs of corresponding bisulfite treated
nucleic acids (DNA upmethylated sense strand, DNA upmethylated
antisense strand, DNA downmethylated sense strand, DNA
downmethylated antisense strand).

| | | | SEQ ID NO: | SEQ ID NO: |
|---|---|---|---|---|
| SEQ ID | SEQ ID NO: | SEQ ID NO: | Down- | Down- |
| NO: | Upmethylated | Upmethylated | methylated | methylated |
| Genomic | Sense | Antisense | Sense | Antisense |
| 1 | 825 | 826 | 2473 | 2474 |
| 2 | 827 | 828 | 2475 | 2476 |
| 3 | 829 | 830 | 2477 | 2478 |
| 4 | 831 | 832 | 2479 | 2480 |
| 5 | 833 | 834 | 2481 | 2482 |
| 6 | 835 | 836 | 2483 | 2484 |
| 7 | 837 | 838 | 2485 | 2486 |
| 8 | 839 | 840 | 2487 | 2488 |
| 9 | 841 | 842 | 2489 | 2490 |
| 10 | 843 | 844 | 2491 | 2492 |
| 11 | 845 | 846 | 2493 | 2494 |
| 12 | 847 | 848 | 2495 | 2496 |
| 13 | 849 | 850 | 2497 | 2498 |
| 14 | 851 | 852 | 2499 | 2500 |
| 15 | 853 | 854 | 2501 | 2502 |
| 16 | 855 | 856 | 2503 | 2504 |
| 17 | 857 | 858 | 2505 | 2506 |
| 18 | 859 | 860 | 2507 | 2508 |
| 19 | 861 | 862 | 2509 | 2510 |
| 20 | 863 | 864 | 2511 | 2512 |
| 21 | 865 | 866 | 2513 | 2514 |
| 22 | 867 | 868 | 2515 | 2516 |
| 23 | 869 | 870 | 2517 | 2518 |
| 24 | 871 | 872 | 2519 | 2520 |
| 25 | 873 | 874 | 2521 | 2522 |
| 26 | 875 | 876 | 2523 | 2524 |
| 27 | 877 | 878 | 2525 | 2526 |
| 28 | 879 | 880 | 2527 | 2528 |
| 29 | 881 | 882 | 2529 | 2530 |
| 30 | 883 | 884 | 2531 | 2532 |
| 31 | 885 | 886 | 2533 | 2534 |
| 32 | 887 | 888 | 2535 | 2536 |
| 33 | 889 | 890 | 2537 | 2538 |
| 34 | 891 | 892 | 2539 | 2540 |
| 35 | 893 | 894 | 2541 | 2542 |
| 36 | 895 | 896 | 2543 | 2544 |
| 37 | 897 | 898 | 2545 | 2546 |
| 38 | 899 | 900 | 2547 | 2548 |
| 39 | 901 | 902 | 2549 | 2550 |
| 40 | 903 | 904 | 2551 | 2552 |
| 41 | 905 | 906 | 2553 | 2554 |
| 42 | 907 | 908 | 2555 | 2556 |
| 43 | 909 | 910 | 2557 | 2558 |
| 44 | 911 | 912 | 2559 | 2560 |
| 45 | 913 | 914 | 2561 | 2562 |
| 46 | 915 | 916 | 2563 | 2564 |
| 47 | 917 | 918 | 2565 | 2566 |
| 48 | 919 | 920 | 2567 | 2568 |
| 49 | 921 | 922 | 2569 | 2570 |
| 50 | 923 | 924 | 2571 | 2572 |
| 51 | 925 | 926 | 2573 | 2574 |
| 52 | 927 | 928 | 2575 | 2576 |
| 53 | 929 | 930 | 2577 | 2578 |
| 54 | 931 | 932 | 2579 | 2580 |
| 55 | 933 | 934 | 2581 | 2582 |
| 56 | 935 | 936 | 2583 | 2584 |
| 57 | 937 | 938 | 2585 | 2586 |
| 58 | 939 | 940 | 2587 | 2588 |
| 59 | 941 | 942 | 2589 | 2590 |
| 60 | 943 | 944 | 2591 | 2592 |
| 61 | 945 | 946 | 2593 | 2594 |
| 62 | 947 | 948 | 2595 | 2596 |
| 63 | 949 | 950 | 2597 | 2598 |
| 64 | 951 | 952 | 2599 | 2600 |
| 65 | 953 | 954 | 2601 | 2602 |
| 66 | 955 | 956 | 2603 | 2604 |
| 67 | 957 | 958 | 2605 | 2606 |
| 68 | 959 | 960 | 2607 | 2608 |
| 69 | 961 | 962 | 2609 | 2610 |
| 70 | 963 | 964 | 2611 | 2612 |
| 71 | 965 | 966 | 2613 | 2614 |
| 72 | 967 | 968 | 2615 | 2616 |
| 73 | 969 | 970 | 2617 | 2618 |
| 74 | 971 | 972 | 2619 | 2620 |
| 75 | 973 | 974 | 2621 | 2622 |
| 76 | 975 | 976 | 2623 | 2624 |
| 77 | 977 | 978 | 2625 | 2626 |
| 78 | 979 | 980 | 2627 | 2628 |
| 79 | 981 | 982 | 2629 | 2630 |
| 80 | 983 | 984 | 2631 | 2632 |
| 81 | 985 | 986 | 2633 | 2634 |
| 82 | 987 | 988 | 2635 | 2636 |
| 83 | 989 | 990 | 2637 | 2638 |
| 84 | 991 | 992 | 2639 | 2640 |
| 85 | 993 | 994 | 2641 | 2642 |
| 86 | 995 | 996 | 2643 | 2644 |
| 87 | 997 | 998 | 2645 | 2646 |
| 88 | 999 | 1000 | 2647 | 2648 |
| 89 | 1001 | 1002 | 2649 | 2650 |
| 90 | 1003 | 1004 | 2651 | 2652 |
| 91 | 1005 | 1006 | 2653 | 2654 |
| 92 | 1007 | 1008 | 2655 | 2656 |
| 93 | 1009 | 1010 | 2657 | 2658 |
| 94 | 1011 | 1012 | 2659 | 2660 |
| 95 | 1013 | 1014 | 2661 | 2662 |
| 96 | 1015 | 1016 | 2663 | 2664 |
| 97 | 1017 | 1018 | 2665 | 2666 |
| 98 | 1019 | 1020 | 2667 | 2668 |
| 99 | 1021 | 1022 | 2669 | 2670 |
| 100 | 1023 | 1024 | 2671 | 2672 |
| 101 | 1025 | 1026 | 2673 | 2674 |
| 102 | 1027 | 1028 | 2675 | 2676 |
| 103 | 1029 | 1030 | 2677 | 2678 |
| 104 | 1031 | 1032 | 2679 | 2680 |
| 105 | 1033 | 1034 | 2681 | 2682 |
| 106 | 1035 | 1036 | 2683 | 2684 |
| 107 | 1037 | 1038 | 2685 | 2686 |
| 108 | 1039 | 1040 | 2687 | 2688 |
| 109 | 1041 | 1042 | 2689 | 2690 |
| 110 | 1043 | 1044 | 2691 | 2692 |
| 111 | 1045 | 1046 | 2693 | 2694 |
| 112 | 1047 | 1048 | 2695 | 2696 |
| 113 | 1049 | 1050 | 2697 | 2698 |
| 114 | 1051 | 1052 | 2699 | 2700 |
| 115 | 1053 | 1054 | 2701 | 2702 |
| 116 | 1055 | 1056 | 2703 | 2704 |
| 117 | 1057 | 1058 | 2705 | 2706 |
| 118 | 1059 | 1060 | 2707 | 2708 |
| 119 | 1061 | 1062 | 2709 | 2710 |
| 120 | 1063 | 1064 | 2711 | 2712 |
| 121 | 1065 | 1066 | 2713 | 2714 |
| 122 | 1067 | 1068 | 2715 | 2716 |
| 123 | 1069 | 1070 | 2717 | 2718 |
| 124 | 1071 | 1072 | 2719 | 2720 |
| 125 | 1073 | 1074 | 2721 | 2722 |
| 126 | 1075 | 1076 | 2723 | 2724 |
| 127 | 1077 | 1078 | 2725 | 2726 |
| 128 | 1079 | 1080 | 2727 | 2728 |
| 129 | 1081 | 1082 | 2729 | 2730 |
| 130 | 1083 | 1084 | 2731 | 2732 |
| 131 | 1085 | 1086 | 2733 | 2734 |
| 132 | 1087 | 1088 | 2735 | 2736 |
| 133 | 1089 | 1090 | 2737 | 2738 |
| 134 | 1091 | 1092 | 2739 | 2740 |
| 135 | 1093 | 1094 | 2741 | 2742 |
| 136 | 1095 | 1096 | 2743 | 2744 |
| 137 | 1097 | 1098 | 2745 | 2746 |
| 138 | 1099 | 1100 | 2747 | 2748 |
| 139 | 1101 | 1102 | 2749 | 2750 |
| 140 | 1103 | 1104 | 2751 | 2752 |
| 141 | 1105 | 1106 | 2753 | 2754 |
| 142 | 1107 | 1108 | 2755 | 2756 |
| 143 | 1109 | 1110 | 2757 | 2758 |
| 144 | 1111 | 1112 | 2759 | 2760 |
| 145 | 1113 | 1114 | 2761 | 2762 |
| 146 | 1115 | 1116 | 2763 | 2764 |
| 147 | 1117 | 1118 | 2765 | 2766 |
| 148 | 1119 | 1120 | 2767 | 2768 |
| 149 | 1121 | 1122 | 2769 | 2770 |
| 150 | 1123 | 1124 | 2771 | 2772 |
| 151 | 1125 | 1126 | 2773 | 2774 |
| 152 | 1127 | 1128 | 2775 | 2776 |
| 153 | 1129 | 1130 | 2777 | 2778 |
| 154 | 1131 | 1132 | 2779 | 2780 |
| 155 | 1133 | 1134 | 2781 | 2782 |
| 156 | 1135 | 1136 | 2783 | 2784 |
| 157 | 1137 | 1138 | 2785 | 2786 |
| 158 | 1139 | 1140 | 2787 | 2788 |
| 159 | 1141 | 1142 | 2789 | 2790 |
| 160 | 1143 | 1144 | 2791 | 2792 |
| 161 | 1145 | 1146 | 2793 | 2794 |
| 162 | 1147 | 1148 | 2795 | 2796 |
| 163 | 1149 | 1150 | 2797 | 2798 |
| 164 | 1151 | 1152 | 2799 | 2800 |
| 165 | 1153 | 1154 | 2801 | 2802 |
| 166 | 1155 | 1156 | 2803 | 2804 |
| 167 | 1157 | 1158 | 2805 | 2806 |
| 168 | 1159 | 1160 | 2807 | 2808 |
| 169 | 1161 | 1162 | 2809 | 2810 |
| 170 | 1163 | 1164 | 2811 | 2812 |
| 171 | 1165 | 1166 | 2813 | 2814 |
| 172 | 1167 | 1168 | 2815 | 2816 |
| 173 | 1169 | 1170 | 2817 | 2818 |
| 174 | 1171 | 1172 | 2819 | 2820 |
| 175 | 1173 | 1174 | 2821 | 2822 |
| 176 | 1175 | 1176 | 2823 | 2824 |
| 177 | 1177 | 1178 | 2825 | 2826 |
| 178 | 1179 | 1180 | 2827 | 2828 |
| 179 | 1181 | 1182 | 2829 | 2830 |
| 180 | 1183 | 1184 | 2831 | 2832 |
| 181 | 1185 | 1186 | 2833 | 2834 |
| 182 | 1187 | 1188 | 2835 | 2836 |
| 183 | 1189 | 1190 | 2837 | 2838 |
| 184 | 1191 | 1192 | 2839 | 2840 |
| 185 | 1193 | 1194 | 2841 | 2842 |
| 186 | 1195 | 1196 | 2843 | 2844 |
| 187 | 1197 | 1198 | 2845 | 2846 |
| 188 | 1199 | 1200 | 2847 | 2848 |
| 189 | 1201 | 1202 | 2849 | 2850 |
| 190 | 1203 | 1204 | 2851 | 2852 |
| 191 | 1205 | 1206 | 2853 | 2854 |
| 192 | 1207 | 1208 | 2855 | 2856 |
| 193 | 1209 | 1210 | 2857 | 2858 |
| 194 | 1211 | 1212 | 2859 | 2860 |
| 195 | 1213 | 1214 | 2861 | 2862 |
| 196 | 1215 | 1216 | 2863 | 2864 |
| 197 | 1217 | 1218 | 2865 | 2866 |
| 198 | 1219 | 1220 | 2867 | 2868 |
| 199 | 1221 | 1222 | 2869 | 2870 |
| 200 | 1223 | 1224 | 2871 | 2872 |
| 201 | 1225 | 1226 | 2873 | 2874 |
| 202 | 1227 | 1228 | 2875 | 2876 |
| 203 | 1229 | 1230 | 2877 | 2878 |
| 204 | 1231 | 1232 | 2879 | 2880 |
| 205 | 1233 | 1234 | 2881 | 2882 |
| 206 | 1235 | 1236 | 2883 | 2884 |
| 207 | 1237 | 1238 | 2885 | 2886 |
| 208 | 1239 | 1240 | 2887 | 2888 |
| 209 | 1241 | 1242 | 2889 | 2890 |
| 210 | 1243 | 1244 | 2891 | 2892 |
| 211 | 1245 | 1246 | 2893 | 2894 |
| 212 | 1247 | 1248 | 2895 | 2896 |
| 213 | 1249 | 1250 | 2897 | 2898 |
| 214 | 1251 | 1252 | 2899 | 2900 |
| 215 | 1253 | 1254 | 2901 | 2902 |
| 216 | 1255 | 1256 | 2903 | 2904 |
| 217 | 1257 | 1258 | 2905 | 2906 |
| 218 | 1259 | 1260 | 2907 | 2908 |
| 219 | 1261 | 1262 | 2909 | 2910 |
| 220 | 1263 | 1264 | 2911 | 2912 |
| 221 | 1265 | 1266 | 2913 | 2914 |
| 222 | 1267 | 1268 | 2915 | 2916 |
| 223 | 1269 | 1270 | 2917 | 2918 |
| 224 | 1271 | 1272 | 2919 | 2920 |
| 225 | 1273 | 1274 | 2921 | 2922 |
| 226 | 1275 | 1276 | 2923 | 2924 |
| 227 | 1277 | 1278 | 2925 | 2926 |
| 228 | 1279 | 1280 | 2927 | 2928 |
| 229 | 1281 | 1282 | 2929 | 2930 |
| 230 | 1283 | 1284 | 2931 | 2932 |
| 231 | 1285 | 1286 | 2933 | 2934 |
| 232 | 1287 | 1288 | 2935 | 2936 |
| 233 | 1289 | 1290 | 2937 | 2938 |
| 234 | 1291 | 1292 | 2939 | 2940 |
| 235 | 1293 | 1294 | 2941 | 2942 |
| 236 | 1295 | 1296 | 2943 | 2944 |
| 237 | 1297 | 1298 | 2945 | 2946 |
| 238 | 1299 | 1300 | 2947 | 2948 |
| 239 | 1301 | 1302 | 2949 | 2950 |
| 240 | 1303 | 1304 | 2951 | 2952 |
| 241 | 1305 | 1306 | 2953 | 2954 |
| 242 | 1307 | 1308 | 2955 | 2956 |
| 243 | 1309 | 1310 | 2957 | 2958 |
| 244 | 1311 | 1312 | 2959 | 2960 |
| 245 | 1313 | 1314 | 2961 | 2962 |
| 246 | 1315 | 1316 | 2963 | 2964 |
| 247 | 1317 | 1318 | 2965 | 2966 |
| 248 | 1319 | 1320 | 2967 | 2968 |
| 249 | 1321 | 1322 | 2969 | 2970 |
| 250 | 1323 | 1324 | 2971 | 2972 |
| 251 | 1325 | 1326 | 2973 | 2974 |
| 252 | 1327 | 1328 | 2975 | 2976 |
| 253 | 1329 | 1330 | 2977 | 2978 |
| 254 | 1331 | 1332 | 2979 | 2980 |
| 255 | 1333 | 1334 | 2981 | 2982 |
| 256 | 1335 | 1336 | 2983 | 2984 |
| 257 | 1337 | 1338 | 2985 | 2986 |
| 258 | 1339 | 1340 | 2987 | 2988 |
| 259 | 1341 | 1342 | 2989 | 2990 |
| 260 | 1343 | 1344 | 2991 | 2992 |
| 261 | 1345 | 1346 | 2993 | 2994 |
| 262 | 1347 | 1348 | 2995 | 2996 |
| 263 | 1349 | 1350 | 2997 | 2998 |
| 264 | 1351 | 1352 | 2999 | 3000 |
| 265 | 1353 | 1354 | 3001 | 3002 |
| 266 | 1355 | 1356 | 3003 | 3004 |
| 267 | 1357 | 1358 | 3005 | 3006 |
| 268 | 1359 | 1360 | 3007 | 3008 |
| 269 | 1361 | 1362 | 3009 | 3010 |
| 270 | 1363 | 1364 | 3011 | 3012 |
| 271 | 1365 | 1366 | 3013 | 3014 |
| 272 | 1367 | 1368 | 3015 | 3016 |
| 273 | 1369 | 1370 | 3017 | 3018 |
| 274 | 1371 | 1372 | 3019 | 3020 |
| 275 | 1373 | 1374 | 3021 | 3022 |
| 276 | 1375 | 1376 | 3023 | 3024 |
| 277 | 1377 | 1378 | 3025 | 3026 |
| 278 | 1379 | 1380 | 3027 | 3028 |
| 279 | 1381 | 1382 | 3029 | 3030 |
| 280 | 1383 | 1384 | 3031 | 3032 |
| 281 | 1385 | 1386 | 3033 | 3034 |
| 282 | 1387 | 1388 | 3035 | 3036 |
| 283 | 1389 | 1390 | 3037 | 3038 |
| 284 | 1391 | 1392 | 3039 | 3040 |
| 285 | 1393 | 1394 | 3041 | 3042 |
| 286 | 1395 | 1396 | 3043 | 3044 |
| 287 | 1397 | 1398 | 3045 | 3046 |
| 288 | 1399 | 1400 | 3047 | 3048 |
| 289 | 1401 | 1402 | 3049 | 3050 |
| 290 | 1403 | 1404 | 3051 | 3052 |
| 291 | 1405 | 1406 | 3053 | 3054 |
| 292 | 1407 | 1408 | 3055 | 3056 |
| 293 | 1409 | 1410 | 3057 | 3058 |
| 294 | 1411 | 1412 | 3059 | 3060 |
| 295 | 1413 | 1414 | 3061 | 3062 |
| 296 | 1415 | 1416 | 3063 | 3064 |
| 297 | 1417 | 1418 | 3065 | 3066 |
| 298 | 1419 | 1420 | 3067 | 3068 |
| 299 | 1421 | 1422 | 3069 | 3070 |
| 300 | 1423 | 1424 | 3071 | 3072 |
| 301 | 1425 | 1426 | 3073 | 3074 |
| 302 | 1427 | 1428 | 3075 | 3076 |
| 303 | 1429 | 1430 | 3077 | 3078 |
| 304 | 1431 | 1432 | 3079 | 3080 |
| 305 | 1433 | 1434 | 3081 | 3082 |
| 306 | 1435 | 1436 | 3083 | 3084 |
| 307 | 1437 | 1438 | 3085 | 3086 |
| 308 | 1439 | 1440 | 3087 | 3088 |
| 309 | 1441 | 1442 | 3089 | 3090 |
| 310 | 1443 | 1444 | 3091 | 3092 |
| 311 | 1445 | 1446 | 3093 | 3094 |
| 312 | 1447 | 1448 | 3095 | 3096 |
| 313 | 1449 | 1450 | 3097 | 3098 |
| 314 | 1451 | 1452 | 3099 | 3100 |
| 315 | 1453 | 1454 | 3101 | 3102 |
| 316 | 1455 | 1456 | 3103 | 3104 |
| 317 | 1457 | 1458 | 3105 | 3106 |
| 318 | 1459 | 1460 | 3107 | 3108 |
| 319 | 1461 | 1462 | 3109 | 3110 |
| 320 | 1463 | 1464 | 3111 | 3112 |
| 321 | 1465 | 1466 | 3113 | 3114 |
| 322 | 1467 | 1468 | 3115 | 3116 |
| 323 | 1469 | 1470 | 3117 | 3118 |
| 324 | 1471 | 1472 | 3119 | 3120 |
| 325 | 1473 | 1474 | 3121 | 3122 |
| 326 | 1475 | 1476 | 3123 | 3124 |
| 327 | 1477 | 1478 | 3125 | 3126 |
| 328 | 1479 | 1480 | 3127 | 3128 |
| 329 | 1481 | 1482 | 3129 | 3130 |
| 330 | 1483 | 1484 | 3131 | 3132 |
| 331 | 1485 | 1486 | 3133 | 3134 |
| 332 | 1487 | 1488 | 3135 | 3136 |
| 333 | 1489 | 1490 | 3137 | 3138 |
| 334 | 1491 | 1492 | 3139 | 3140 |
| 335 | 1493 | 1494 | 3141 | 3142 |
| 336 | 1495 | 1496 | 3143 | 3144 |
| 337 | 1497 | 1498 | 3145 | 3146 |
| 338 | 1499 | 1500 | 3147 | 3148 |
| 339 | 1501 | 1502 | 3149 | 3150 |
| 340 | 1503 | 1504 | 3151 | 3152 |
| 341 | 1505 | 1506 | 3153 | 3154 |
| 342 | 1507 | 1508 | 3155 | 3156 |
| 343 | 1509 | 1510 | 3157 | 3158 |
| 344 | 1511 | 1512 | 3159 | 3160 |
| 345 | 1513 | 1514 | 3161 | 3162 |
| 346 | 1515 | 1516 | 3163 | 3164 |
| 347 | 1517 | 1518 | 3165 | 3166 |
| 348 | 1519 | 1520 | 3167 | 3168 |
| 349 | 1521 | 1522 | 3169 | 3170 |
| 350 | 1523 | 1524 | 3171 | 3172 |
| 351 | 1525 | 1526 | 3173 | 3174 |
| 352 | 1527 | 1528 | 3175 | 3176 |
| 353 | 1529 | 1530 | 3177 | 3178 |
| 354 | 1531 | 1532 | 3179 | 3180 |
| 355 | 1533 | 1534 | 3181 | 3182 |
| 356 | 1535 | 1536 | 3183 | 3184 |
| 357 | 1537 | 1538 | 3185 | 3186 |
| 358 | 1539 | 1540 | 3187 | 3188 |
| 359 | 1541 | 1542 | 3189 | 3190 |
| 360 | 1543 | 1544 | 3191 | 3192 |
| 361 | 1545 | 1546 | 3193 | 3194 |
| 362 | 1547 | 1548 | 3195 | 3196 |
| 363 | 1549 | 1550 | 3197 | 3198 |
| 364 | 1551 | 1552 | 3199 | 3200 |
| 365 | 1553 | 1554 | 3201 | 3202 |
| 366 | 1555 | 1556 | 3203 | 3204 |
| 367 | 1557 | 1558 | 3205 | 3206 |
| 368 | 1559 | 1560 | 3207 | 3208 |
| 369 | 1561 | 1562 | 3209 | 3210 |
| 370 | 1563 | 1564 | 3211 | 3212 |
| 371 | 1565 | 1566 | 3213 | 3214 |
| 372 | 1567 | 1568 | 3215 | 3216 |
| 373 | 1569 | 1570 | 3217 | 3218 |
| 374 | 1571 | 1572 | 3219 | 3220 |
| 375 | 1573 | 1574 | 3221 | 3222 |
| 376 | 1575 | 1576 | 3223 | 3224 |
| 377 | 1577 | 1578 | 3225 | 3226 |
| 378 | 1579 | 1580 | 3227 | 3228 |
| 379 | 1581 | 1582 | 3229 | 3230 |
| 380 | 1583 | 1584 | 3231 | 3232 |
| 381 | 1585 | 1586 | 3233 | 3234 |
| 382 | 1587 | 1588 | 3235 | 3236 |
| 383 | 1589 | 1590 | 3237 | 3238 |
| 384 | 1591 | 1592 | 3239 | 3240 |
| 385 | 1593 | 1594 | 3241 | 3242 |
| 386 | 1595 | 1596 | 3243 | 3244 |
| 387 | 1597 | 1598 | 3245 | 3246 |
| 388 | 1599 | 1600 | 3247 | 3248 |
| 389 | 1601 | 1602 | 3249 | 3250 |
| 390 | 1603 | 1604 | 3251 | 3252 |
| 391 | 1605 | 1606 | 3253 | 3254 |
| 392 | 1607 | 1608 | 3255 | 3256 |
| 393 | 1609 | 1610 | 3257 | 3258 |
| 394 | 1611 | 1612 | 3259 | 3260 |
| 395 | 1613 | 1614 | 3261 | 3262 |
| 396 | 1615 | 1616 | 3263 | 3264 |
| 397 | 1617 | 1618 | 3265 | 3266 |
| 398 | 1619 | 1620 | 3267 | 3268 |
| 399 | 1621 | 1622 | 3269 | 3270 |
| 400 | 1623 | 1624 | 3271 | 3272 |
| 401 | 1625 | 1626 | 3273 | 3274 |
| 402 | 1627 | 1628 | 3275 | 3276 |
| 403 | 1629 | 1630 | 3277 | 3278 |
| 404 | 1631 | 1632 | 3279 | 3280 |
| 405 | 1633 | 1634 | 3281 | 3282 |
| 406 | 1635 | 1636 | 3283 | 3284 |
| 407 | 1637 | 1638 | 3285 | 3286 |
| 408 | 1639 | 1640 | 3287 | 3288 |
| 409 | 1641 | 1642 | 3289 | 3290 |
| 410 | 1643 | 1644 | 3291 | 3292 |
| 411 | 1645 | 1646 | 3293 | 3294 |
| 412 | 1647 | 1648 | 3295 | 3296 |

TABLE 4
Overview of SEQ ID NO of the genes or genomic regions and of
corresponding amplificates amplified by use of the corresponding
Forward and Reverse Primer named by their SEQ ID NO.

| SEQ | Forward Primer | Reverse Primer | SEQ |
|---|---|---|---|
| ID NO: | SEQ ID | SEQ ID | ID NO: |
| Genomic | NO: | NO: | Genomic |
| 413 | 4121 | 4122 | 1 |
| 414 | 4123 | 4124 | 2 |
| 415 | 4125 | 4126 | 3 |
| 416 | 4127 | 4128 | 4 |
| 417 | 4129 | 4130 | 5 |
| 418 | 4131 | 4132 | 6 |
| 419 | 4133 | 4134 | 7 |
| 420 | 4135 | 4136 | 8 |
| 421 | 4137 | 4138 | 9 |
| 422 | 4139 | 4140 | 10 |
| 423 | 4141 | 4142 | 11 |
| 424 | 4143 | 4144 | 12 |
| 425 | 4145 | 4146 | 13 |
| 426 | 4147 | 4148 | 14 |
| 427 | 4149 | 4150 | 15 |
| 428 | 4151 | 4152 | 16 |
| 429 | 4153 | 4154 | 17 |
| 430 | 4155 | 4156 | 18 |
| 431 | 4157 | 4158 | 19 |
| 432 | 4159 | 4160 | 20 |
| 433 | 4161 | 4162 | 21 |
| 434 | 4163 | 4164 | 22 |
| 435 | 4165 | 4166 | 23 |
| 436 | 4167 | 4168 | 24 |
| 437 | 4169 | 4170 | 25 |
| 438 | 4171 | 4172 | 26 |
| 439 | 4173 | 4174 | 27 |
| 440 | 4175 | 4176 | 28 |
| 441 | 4177 | 4178 | 29 |
| 442 | 4179 | 4180 | 30 |
| 443 | 4181 | 4182 | 31 |
| 444 | 4183 | 4184 | 32 |
| 445 | 4185 | 4186 | 33 |
| 446 | 4187 | 4188 | 34 |
| 447 | 4189 | 4190 | 35 |
| 448 | 4191 | 4192 | 36 |
| 449 | 4193 | 4194 | 37 |
| 450 | 4195 | 4196 | 38 |
| 451 | 4197 | 4198 | 39 |
| 452 | 4199 | 4200 | 40 |
| 453 | 4201 | 4202 | 41 |
| 454 | 4203 | 4204 | 42 |
| 455 | 4205 | 4206 | 43 |
| 456 | 4207 | 4208 | 44 |
| 457 | 4209 | 4210 | 45 |
| 458 | 4211 | 4212 | 46 |
| 459 | 4213 | 4214 | 47 |
| 460 | 4215 | 4216 | 48 |
| 461 | 4217 | 4218 | 49 |
| 462 | 4219 | 4220 | 50 |
| 463 | 4221 | 4222 | 51 |
| 464 | 4223 | 4224 | 52 |
| 465 | 4225 | 4226 | 53 |
| 466 | 4227 | 4228 | 54 |
| 467 | 4229 | 4230 | 55 |
| 468 | 4231 | 4232 | 56 |
| 469 | 4233 | 4234 | 57 |
| 470 | 4235 | 4236 | 58 |
| 471 | 4237 | 4238 | 59 |
| 472 | 4239 | 4240 | 60 |
| 473 | 4241 | 4242 | 61 |
| 474 | 4243 | 4244 | 62 |
| 475 | 4245 | 4246 | 63 |
| 476 | 4247 | 4248 | 64 |
| 477 | 4249 | 4250 | 65 |
| 478 | 4251 | 4252 | 66 |
| 479 | 4253 | 4254 | 67 |
| 480 | 4255 | 4256 | 68 |
| 481 | 4257 | 4258 | 69 |
| 482 | 4259 | 4260 | 70 |
| 483 | 4261 | 4262 | 71 |
| 484 | 4263 | 4264 | 72 |
| 485 | 4265 | 4266 | 73 |
| 486 | 4267 | 4268 | 74 |
| 487 | 4269 | 4270 | 75 |
| 488 | 4271 | 4272 | 76 |
| 489 | 4273 | 4274 | 77 |
| 490 | 4275 | 4276 | 78 |
| 491 | 4277 | 4278 | 79 |
| 492 | 4279 | 4280 | 80 |
| 493 | 4281 | 4282 | 81 |
| 494 | 4283 | 4284 | 82 |
| 495 | 4285 | 4286 | 83 |
| 496 | 4287 | 4288 | 84 |
| 497 | 4289 | 4290 | 85 |
| 498 | 4291 | 4292 | 86 |
| 499 | 4293 | 4294 | 87 |
| 500 | 4295 | 4296 | 88 |
| 501 | 4297 | 4298 | 89 |
| 502 | 4299 | 4300 | 90 |
| 503 | 4301 | 4302 | 91 |
| 504 | 4303 | 4304 | 92 |
| 505 | 4305 | 4306 | 93 |
| 506 | 4307 | 4308 | 94 |
| 507 | 4309 | 4310 | 95 |
| 508 | 4311 | 4312 | 96 |
| 509 | 4313 | 4314 | 97 |
| 510 | 4315 | 4316 | 98 |
| 511 | 4317 | 4318 | 99 |
| 512 | 4319 | 4320 | 100 |
| 513 | 4321 | 4322 | 101 |
| 514 | 4323 | 4324 | 102 |
| 515 | 4325 | 4326 | 103 |
| 516 | 4327 | 4328 | 104 |
| 517 | 4329 | 4330 | 105 |
| 518 | 4331 | 4332 | 106 |
| 519 | 4333 | 4334 | 107 |
| 520 | 4335 | 4336 | 108 |
| 521 | 4337 | 4338 | 109 |
| 522 | 4339 | 4340 | 110 |
| 523 | 4341 | 4342 | 111 |
| 524 | 4343 | 4344 | 112 |
| 525 | 4345 | 4346 | 113 |
| 526 | 4347 | 4348 | 114 |
| 527 | 4349 | 4350 | 115 |
| 528 | 4351 | 4352 | 116 |
| 529 | 4353 | 4354 | 117 |
| 530 | 4355 | 4356 | 118 |
| 531 | 4357 | 4358 | 119 |
| 532 | 4359 | 4360 | 120 |
| 533 | 4361 | 4362 | 121 |
| 534 | 4363 | 4364 | 122 |
| 535 | 4365 | 4366 | 123 |
| 536 | 4367 | 4368 | 124 |
| 537 | 4369 | 4370 | 125 |
| 538 | 4371 | 4372 | 126 |
| 539 | 4373 | 4374 | 127 |
| 540 | 4375 | 4376 | 128 |
| 541 | 4377 | 4378 | 129 |
| 542 | 4379 | 4380 | 130 |
| 543 | 4381 | 4382 | 131 |
| 544 | 4383 | 4384 | 132 |
| 545 | 4385 | 4386 | 133 |
| 546 | 4387 | 4388 | 134 |
| 547 | 4389 | 4390 | 135 |
| 548 | 4391 | 4392 | 136 |
| 549 | 4393 | 4394 | 137 |
| 550 | 4395 | 4396 | 138 |
| 551 | 4397 | 4398 | 139 |
| 552 | 4399 | 4400 | 140 |
| 553 | 4401 | 4402 | 141 |
| 554 | 4403 | 4404 | 142 |
| 555 | 4405 | 4406 | 143 |
| 556 | 4407 | 4408 | 144 |
| 557 | 4409 | 4410 | 145 |
| 558 | 4411 | 4412 | 146 |
| 559 | 4413 | 4414 | 147 |
| 560 | 4415 | 4416 | 148 |
| 561 | 4417 | 4418 | 149 |
| 562 | 4419 | 4420 | 150 |
| 563 | 4421 | 4422 | 151 |
| 564 | 4423 | 4424 | 152 |
| 565 | 4425 | 4426 | 153 |
| 566 | 4427 | 4428 | 154 |
| 567 | 4429 | 4430 | 155 |
| 568 | 4431 | 4432 | 156 |
| 569 | 4433 | 4434 | 157 |
| 570 | 4435 | 4436 | 158 |
| 571 | 4437 | 4438 | 159 |
| 572 | 4439 | 4440 | 160 |
| 573 | 4441 | 4442 | 161 |
| 574 | 4443 | 4444 | 162 |
| 575 | 4445 | 4446 | 163 |
| 576 | 4447 | 4448 | 164 |
| 577 | 4449 | 4450 | 165 |
| 578 | 4451 | 4452 | 166 |
| 579 | 4453 | 4454 | 167 |
| 580 | 4455 | 4456 | 168 |
| 581 | 4457 | 4458 | 169 |
| 582 | 4459 | 4460 | 170 |
| 583 | 4461 | 4462 | 171 |
| 584 | 4463 | 4464 | 172 |
| 585 | 4465 | 4466 | 173 |
| 586 | 4467 | 4468 | 174 |
| 587 | 4469 | 4470 | 175 |
| 588 | 4471 | 4472 | 176 |
| 589 | 4473 | 4474 | 177 |
| 590 | 4475 | 4476 | 178 |
| 591 | 4477 | 4478 | 179 |
| 592 | 4479 | 4480 | 180 |
| 593 | 4481 | 4482 | 181 |
| 594 | 4483 | 4484 | 182 |
| 595 | 4485 | 4486 | 183 |
| 596 | 4487 | 4488 | 184 |
| 597 | 4489 | 4490 | 185 |
| 598 | 4491 | 4492 | 186 |
| 599 | 4493 | 4494 | 187 |
| 600 | 4495 | 4496 | 188 |
| 601 | 4497 | 4498 | 189 |
| 602 | 4499 | 4500 | 190 |
| 603 | 4501 | 4502 | 191 |
| 604 | 4503 | 4504 | 192 |
| 605 | 4505 | 4506 | 193 |
| 606 | 4507 | 4508 | 194 |
| 607 | 4509 | 4510 | 195 |
| 608 | 4511 | 4512 | 196 |
| 609 | 4513 | 4514 | 197 |
| 610 | 4515 | 4516 | 198 |
| 611 | 4517 | 4518 | 199 |
| 612 | 4519 | 4520 | 200 |
| 613 | 4521 | 4522 | 201 |
| 614 | 4523 | 4524 | 202 |
| 615 | 4525 | 4526 | 203 |
| 616 | 4527 | 4528 | 204 |
| 617 | 4529 | 4530 | 205 |
| 618 | 4531 | 4532 | 206 |
| 619 | 4533 | 4534 | 207 |
| 620 | 4535 | 4536 | 208 |
| 621 | 4537 | 4538 | 209 |
| 622 | 4539 | 4540 | 210 |
| 623 | 4541 | 4542 | 211 |
| 624 | 4543 | 4544 | 212 |
| 625 | 4545 | 4546 | 213 |
| 626 | 4547 | 4548 | 214 |
| 627 | 4549 | 4550 | 215 |
| 628 | 4551 | 4552 | 216 |
| 629 | 4553 | 4554 | 217 |
| 630 | 4555 | 4556 | 218 |
| 631 | 4557 | 4558 | 219 |
| 632 | 4559 | 4560 | 220 |
| 633 | 4561 | 4562 | 221 |
| 634 | 4563 | 4564 | 222 |
| 635 | 4565 | 4566 | 223 |
| 636 | 4567 | 4568 | 224 |
| 637 | 4569 | 4570 | 225 |
| 638 | 4571 | 4572 | 226 |
| 639 | 4573 | 4574 | 227 |
| 640 | 4575 | 4576 | 228 |
| 641 | 4577 | 4578 | 229 |
| 642 | 4579 | 4580 | 230 |
| 643 | 4581 | 4582 | 231 |
| 644 | 4583 | 4584 | 232 |
| 645 | 4585 | 4586 | 233 |
| 646 | 4587 | 4588 | 234 |
| 647 | 4589 | 4590 | 235 |
| 648 | 4591 | 4592 | 236 |
| 649 | 4593 | 4594 | 237 |
| 650 | 4595 | 4596 | 238 |
| 651 | 4597 | 4598 | 239 |
| 652 | 4599 | 4600 | 240 |
| 653 | 4601 | 4602 | 241 |
| 654 | 4603 | 4604 | 242 |
| 655 | 4605 | 4606 | 243 |
| 656 | 4607 | 4608 | 244 |
| 657 | 4609 | 4610 | 245 |
| 658 | 4611 | 4612 | 246 |
| 659 | 4613 | 4614 | 247 |
| 660 | 4615 | 4616 | 248 |
| 661 | 4617 | 4618 | 249 |
| 662 | 4619 | 4620 | 250 |
| 663 | 4621 | 4622 | 251 |
| 664 | 4623 | 4624 | 252 |
| 665 | 4625 | 4626 | 253 |
| 666 | 4627 | 4628 | 254 |
| 667 | 4629 | 4630 | 255 |
| 668 | 4631 | 4632 | 256 |
| 669 | 4633 | 4634 | 257 |
| 670 | 4635 | 4636 | 258 |
| 671 | 4637 | 4638 | 259 |
| 672 | 4639 | 4640 | 260 |
| 673 | 4641 | 4642 | 261 |
| 674 | 4643 | 4644 | 262 |
| 675 | 4645 | 4646 | 263 |
| 676 | 4647 | 4648 | 264 |
| 677 | 4649 | 4650 | 265 |
| 678 | 4651 | 4652 | 266 |
| 679 | 4653 | 4654 | 267 |
| 680 | 4655 | 4656 | 268 |
| 681 | 4657 | 4658 | 269 |
| 682 | 4659 | 4660 | 270 |
| 683 | 4661 | 4662 | 271 |
| 684 | 4663 | 4664 | 272 |
| 685 | 4665 | 4666 | 273 |
| 686 | 4667 | 4668 | 274 |
| 687 | 4669 | 4670 | 275 |
| 688 | 4671 | 4672 | 276 |
| 689 | 4673 | 4674 | 277 |
| 690 | 4675 | 4676 | 278 |
| 691 | 4677 | 4678 | 279 |
| 692 | 4679 | 4680 | 280 |
| 693 | 4681 | 4682 | 281 |
| 694 | 4683 | 4684 | 282 |
| 695 | 4685 | 4686 | 283 |
| 696 | 4687 | 4688 | 284 |
| 697 | 4689 | 4690 | 285 |
| 698 | 4691 | 4692 | 286 |
| 699 | 4693 | 4694 | 287 |
| 700 | 4695 | 4696 | 288 |
| 701 | 4697 | 4698 | 289 |
| 702 | 4699 | 4700 | 290 |
| 703 | 4701 | 4702 | 291 |
| 704 | 4703 | 4704 | 292 |
| 705 | 4705 | 4706 | 293 |
| 706 | 4707 | 4708 | 294 |
| 707 | 4709 | 4710 | 295 |
| 708 | 4711 | 4712 | 296 |
| 709 | 4713 | 4714 | 297 |
| 710 | 4715 | 4716 | 298 |
| 711 | 4717 | 4718 | 299 |
| 712 | 4719 | 4720 | 300 |
| 713 | 4721 | 4722 | 301 |
| 714 | 4723 | 4724 | 302 |
| 715 | 4725 | 4726 | 303 |
| 716 | 4727 | 4728 | 304 |
| 717 | 4729 | 4730 | 305 |
| 718 | 4731 | 4732 | 306 |
| 719 | 4733 | 4734 | 307 |
| 720 | 4735 | 4736 | 308 |
| 721 | 4737 | 4738 | 309 |
| 722 | 4739 | 4740 | 310 |
| 723 | 4741 | 4742 | 311 |
| 724 | 4743 | 4744 | 312 |
| 725 | 4745 | 4746 | 313 |
| 726 | 4747 | 4748 | 314 |
| 727 | 4749 | 4750 | 315 |
| 728 | 4751 | 4752 | 316 |
| 729 | 4753 | 4754 | 317 |
| 730 | 4755 | 4756 | 318 |
| 731 | 4757 | 4758 | 319 |
| 732 | 4759 | 4760 | 320 |
| 733 | 4761 | 4762 | 321 |
| 734 | 4763 | 4764 | 322 |
| 735 | 4765 | 4766 | 323 |
| 736 | 4767 | 4768 | 324 |
| 737 | 4769 | 4770 | 325 |
| 738 | 4771 | 4772 | 326 |
| 739 | 4773 | 4774 | 327 |
| 740 | 4775 | 4776 | 328 |
| 741 | 4777 | 4778 | 329 |
| 742 | 4779 | 4780 | 330 |
| 743 | 4781 | 4782 | 331 |
| 744 | 4783 | 4784 | 332 |
| 745 | 4785 | 4786 | 333 |
| 746 | 4787 | 4788 | 334 |
| 747 | 4789 | 4790 | 335 |
| 748 | 4791 | 4792 | 336 |
| 749 | 4793 | 4794 | 337 |
| 750 | 4795 | 4796 | 338 |
| 751 | 4797 | 4798 | 339 |
| 752 | 4799 | 4800 | 340 |
| 753 | 4801 | 4802 | 341 |
| 754 | 4803 | 4804 | 342 |
| 755 | 4805 | 4806 | 343 |
| 756 | 4807 | 4808 | 344 |
| 757 | 4809 | 4810 | 345 |
| 758 | 4811 | 4812 | 346 |
| 759 | 4813 | 4814 | 347 |
| 760 | 4815 | 4816 | 348 |
| 761 | 4817 | 4818 | 349 |
| 762 | 4819 | 4820 | 350 |
| 763 | 4821 | 4822 | 351 |
| 764 | 4823 | 4824 | 352 |
| 765 | 4825 | 4826 | 353 |
| 766 | 4827 | 4828 | 354 |
| 767 | 4829 | 4830 | 355 |
| 768 | 4831 | 4832 | 356 |
| 769 | 4833 | 4834 | 357 |
| 770 | 4835 | 4836 | 358 |
| 771 | 4837 | 4838 | 359 |
| 772 | 4839 | 4840 | 360 |
| 773 | 4841 | 4842 | 361 |
| 774 | 4843 | 4844 | 362 |
| 775 | 4845 | 4846 | 363 |
| 776 | 4847 | 4848 | 364 |
| 777 | 4849 | 4850 | 365 |
| 778 | 4851 | 4852 | 366 |
| 779 | 4853 | 4854 | 367 |
| 780 | 4855 | 4856 | 368 |
| 781 | 4857 | 4858 | 369 |
| 782 | 4859 | 4860 | 370 |
| 783 | 4861 | 4862 | 371 |
| 784 | 4863 | 4864 | 372 |
| 785 | 4865 | 4866 | 373 |
| 786 | 4867 | 4868 | 374 |
| 787 | 4869 | 4870 | 375 |
| 788 | 4871 | 4872 | 376 |
| 789 | 4873 | 4874 | 377 |
| 790 | 4875 | 4876 | 378 |
| 791 | 4877 | 4878 | 379 |
| 792 | 4879 | 4880 | 380 |
| 793 | 4881 | 4882 | 381 |
| 794 | 4883 | 4884 | 382 |
| 795 | 4885 | 4886 | 383 |
| 796 | 4887 | 4888 | 384 |
| 797 | 4889 | 4890 | 385 |
| 798 | 4891 | 4892 | 386 |
| 799 | 4893 | 4894 | 387 |
| 800 | 4895 | 4896 | 388 |
| 801 | 4897 | 4898 | 389 |
| 802 | 4899 | 4900 | 390 |
| 803 | 4901 | 4902 | 391 |
| 804 | 4903 | 4904 | 392 |
| 805 | 4905 | 4906 | 393 |
| 806 | 4907 | 4908 | 394 |
| 807 | 4909 | 4910 | 395 |
| 808 | 4911 | 4912 | 396 |
| 809 | 4913 | 4914 | 397 |
| 810 | 4915 | 4916 | 398 |
| 811 | 4917 | 4918 | 399 |
| 812 | 4919 | 4920 | 400 |
| 813 | 4921 | 4922 | 401 |
| 814 | 4923 | 4924 | 402 |
| 815 | 4925 | 4926 | 403 |
| 816 | 4927 | 4928 | 404 |
| 817 | 4929 | 4930 | 405 |
| 818 | 4931 | 4932 | 406 |
| 819 | 4933 | 4934 | 407 |
| 820 | 4935 | 4936 | 408 |
| 821 | 4937 | 4938 | 409 |
| 822 | 4939 | 4940 | 410 |
| 823 | 4941 | 4942 | 411 |
| 824 | 4943 | 4944 | 412 |

TABLE 5
Overview of genes or genomic regions and the corresponding SEQ ID NOs, HUGO IDs,
Aliases, Reference Sequence IDs, Ensemble Gene IDs and Entrez IDs.

| SEQ | | | | | | |
|---|---|---|---|---|---|---|
| ID | | | | | | |
| NO: | | | | | | |
| Ge- | | | | | | |
| nom- | | | | | Ensembl Gene | Entrez Gene |
| ic | Gene | HUGO ID | Aliases | RefSeq ID | ID | ID |
| 413 | FLOT1, flotillin 1, | FLOT1 | | NM_005803 | ENSG00000137312 | 10211 |
| | ENSG00000137312 | | | | | |
| 414 | C6orf25, chromosome | C6orf25 | G6b; | NM_025260 | ENSG00000096148 | 80739 |
| | 6 open reading frame | | NG31 | | | |
| | 25, | | | | | |
| | ENSG00000096148 | | | | | |
| 415 | VARS, valyl-tRNA | VARS | G7A; | NM_006295 | ENSG00000096171 | 7407 |
| | synthetase, | | VARS2 | | | |
| | ENSG00000096171 | | | | | |
| 416 | major | HLA- | DPB1; | NM_002121 | OTTHUMG00000031076 | 3115 |
| | histocompatibility | DPB1 | HLA- | | | |
| | complex, class II, DP | | DP1B; | | | |
| | beta 1, | | MHC | | | |
| | OTTHUMG00000031076, | | DPB1 | | | |
| | HLA-DPB1 | | | | | |
| 417 | HLA-DRB5, major | HLA- | | NG_002432 | OTTHUMG00000031027 | 3127 |
| | histocompatibility | DRB5 | | | | |
| | complex, class II, DR | | | | | |
| | beta 5, | | | | | |
| | OTTHUMG00000031027 | | | | | |
| 418 | COL11A2, collagen, | COL11A2 | HKE5; | NM_080679 | OTTHUMG00000031036 | 1302 |
| | type XI, alpha 2, | | PARP; | | | |
| | OTTHUMG00000031036 | | STL3; | | | |
| | | | DFNA13; | | | |
| | | | DFNB53 | | | |
| 419 | PRAME, Melanoma | PRAME | MAPE; | NG_000002 | ENSG00000185686 | 23532 |
| | antigen preferentially | | OIP4 | | | |
| | expressed in tumors | | | | | |
| | (Preferentially | | | | | |
| | expressed antigen of | | | | | |
| | melanoma) (OPA- | | | | | |
| | interacting protein 4) | | | | | |
| | (OIP4), ENSG00000185686 | | | | | |
| 420 | ZNRF3 protein | ZNRF3 | KIAA1133, | XM_290972, | ENSG00000183579 | 84133 |
| | (Fragment), | | BK747E2.3, | XP_290972 | | |
| | ENSG00000183579, | | FLJ22057, | | | |
| | ZNRF3 zinc and ring | | RNF203 | | | |
| | finger 3 (ZNRF3) | | | | | |
| 421 | AP000357.2 (Vega | AP000357.2 | Em: AP000357.C22.2 | No | OTTHUMG00000030571 | No Available |
| | gene ID), Pseudogene | (Vega | | Available | | |
| | | gene ID) | | | | |
| 422 | AP000357.3 (Vega | AP000357.3 | Em: AP000357.C22.3 | No | OTTHUMG00000030574 | No Available |
| | gene ID), Pseudogene | (Vega | | Available | | |
| | | gene ID) | | | | |
| 423 | solute carrier family 7 | SLC7A4 | CAT4; | NM_004173 | OTTHUMG00000030129 | 6545 |
| | (cationic amino acid | | CAT-4; | | | |
| | transporter, y+ | | HCAT3; | | | |
| | system), member 4, | | MGC129976; | | | |
| | OTTHUMG00000030129, | | MGC129977 | | | |
| | SLC7A4 ( | | | | | |
| 424 | Myosin-18B (Myosin | MYO18B | BK125H2.1 | NM_014550 | ENSG00000133454 | 84700 |
| | XVIIIb), | | | | | |
| | ENSG00000133454, | | | | | |
| | MYO18B | | | | | |
| 425 | Q6ICL0_HUMAN | Q6ICL0_HUMAN | Em: AC006547.7, | | ENSG00000184004 | 150197 |
| | (Predicted | (Predicted | FLJ32575 | | | |
| | UniProt/TrEMBL ID), | UniProt/TrEMBL | | | | |
| | hypothetical protein | ID) | | | | |
| | FLJ3257; | | | | | |
| | ENSG00000184004 | | | | | |
| 426 | FBLN1; fibulin 1; | FBLN1 | FBLN | NM_001996 | ENSG00000077942 | 2192 |
| | ENSG00000077942 | | | | | |
| 427 | CYP2D6; cytochrome | CYP2D6 | CPD6; | NG_003180 | ENSG00000100197 | 1565 |
| | P450, family 2, | | CYP2D; | | | |
| | subfamily D, | | CYP2D@; | | | |
| | polypeptide 6; | | CYP2DL1; | | | |
| | ENSG00000100197 | | 450C2D; | | | |
| | | | P450-DB1; | | | |
| | | | MGC120389; | | | |
| | | | MGC120390 | | | |
| 428 | AC008132.9 (Vega | AC008132.9 | AC008103.3; | No | OTTHUMG00000030688 | No Available |
| | gene ID); Pseudogene; | (Vega | Em: AC008132.C22.2 | Available | | |
| | OTTHUMG00000030688 | gene ID) | | | | |
| 429 | glycoprotein Ib | GP1BB | | NM_000407 | OTTHUMT00000075045 | 2812 |
| | (platelet), beta | | | | | |
| | polypeptide, | | | | | |
| | OTTHUMT00000075045, | | | | | |
| | GP1BB-001 | | | | | |
| 430 | no gene associated | | | | | |
| 431 | AC006548.8 (Vega | AC006548.8 | Em: AC006548.C22.8 | No | OTTHUMG00000030274 | No Available |
| | gene ID) | (Vega | | Available | | |
| | | gene ID) | | | | |
| 432 | OTTHUMG00000030650, | AC005399.2 | Em: AC005399.C22.2 | No | OTTHUMG00000030650 | No Available |
| | AC005399.2, | (Vega | | Available | | |
| | putativer processed | gene ID) | | | | |
| | transcribed | | | | | |
| 433 | topoisomerase (DNA) | TOP3B | | NM_003935 | OTTHUMG00000030764 | 8940 |
| | III beta, | | | | | |
| | OTTHUMG00000030764, | | | | | |
| | TOP3B ( | | | | | |
| 434 | no gene associated | | | | | |
| 435 | KB-1269D1.3 (Vega | KB- | Em: AP000344.C22.3 | No | OTTHUMG00000030694 | No Available |
| | gene ID); Pseudogene; | 1269D1.3 | | Available | | |
| | OTTHUMG00000030694 | (Vega gene | | | | |
| | | ID) | | | | |
| 436 | GPR24; G protein- | GPR24 | SLC1; | NM_005297 | ENSG00000128285 | 2847 |
| | coupled receptor 24; | | MCHR1; | | | |
| | ENSG00000128285 | | MGC32129 | | | |
| 437 | GAL3ST1; galactose- | GAL3ST1 | CST | NM_004861 | ENSG00000128242 | 9514 |
| | 3-O-sulfotransferase 1; | | | | | |
| | ENSG00000128242 | | | | | |
| 438 | Cat eye syndrome | CECR5 | | NM_017829 | ENSG00000069998 | 27440 |
| | critical region protein | | | | | |
| | 5 precursor, | | | | | |
| | ENSG00000069998, | | | | | |
| | CECR5 and (head to | | | | | |
| | head) | | | | | |
| | OTTHUMG00000030478, | | | | | |
| | AC006946.7 | | | | | |
| 439 | HORMAD2; HORMA | HORMAD2 | MGC26710 | NM_152510 | ENSG00000176635 | 150280 |
| | domain containing 2; | | | | | |
| | ENSG00000176635 | | | | | |
| 440 | OTTHUMG00000030922, | RP3- | Em: AC002378.C22.2 | No | OTTHUMG00000030922 | No Available |
| | RP3-438O4.2 | 438O4.2 | | Available | | |
| | | (Vega_gene | | | | |
| | | ID) | | | | |
| 441 | NP_997357.1 (RefSeq | NP_997357.1 | FLJ42953 | NM_207474 | ENSG00000169668 | 400892 |
| | peptide ID); | (RefSeq | | | | |
| | ENSG00000169668 | peptide ID) | | | | |
| 442 | OTTHUMG00000030574, | AP000357.3 | Em: AP000357.C22.3 | | OTTHUMG00000030574 | |
| | AP000357.3, | | | | | |
| | novel pseudogene | | | | | |
| 443 | LA16c-4G1.2 (Vega | LA16c- | Em: AP000522.C22.2 | No | OTTHUMG00000030832 | No Available |
| | gene ID); Pseudogene; | 4G1.2 | | Available | | |
| | OTTHUMG00000030832 | (Vega gene | | | | |
| | | ID) | | | | |
| 444 | KB-226F1.11 (Vega | KB- | Em: | No | OTTHUMG00000030123 | No Available |
| | gene ID), embryonic | 226F1.11 | AP000351.C22.11 | Available | | |
| | marker, | (Vega gene | | | | |
| | OTTHUMG00000030123 | ID) | | | | |
| 445 | OTTHUMG00000030780, | CTA- | bK373H7.C22.4 | No | OTTHUMG00000030780 | No Available |
| | CTA-373H7.4, | 373H7.4 | | Available | | |
| | novel pseudogene | (Vega gene | | | | |
| | | ID) | | | | |
| 446 | RP1-47A17.8 (Vega | RP1- | dJ47A17.C22.8 | No | OTTHUMG00000030878 | No Available |
| | gene ID); | 47A17.8 | | Available | | |
| | OTTHUMG00000030878 | (Vega gene | | | | |
| | | ID); | | | | |
| 447 | RP4-539M6.7 (Vega | RP4- | Em: AC004832.C22.7 | No | OTTHUMG00000030918 | No Available |
| | gene ID); Pseudogene; | 539M6.7 | | Available | | |
| | OTTHUMG00000030918 | (Vega gene | | | | |
| | | ID) | | | | |
| 448 | CSDC2; cold shock | CSDC2 | PIPPIN; | NM_014460 | ENSG00000172346 | 27254 |
| | domain containing C2, | | dJ347H13.2 | | | |
| | RNA binding; | | | | | |
| | ENSG00000172346 | | | | | |
| 449 | Gamma-parvin, | PARVG | | NM_022141 | ENSG00000138964 | 64098 |
| | ENSG00000138964, | | | | | |
| | PARVG | | | | | |
| 450 | OTTHUMG00000030167, | CTA- | bK243E7.C22.3 | No | OTTHUMG00000030167 | No Available |
| | CTA-243E7.3 | 243E7.3 | | Available | | |
| | | (Vega gene | | | | |
| | | ID) | | | | |
| 451 | Oncostatin M | OSM | MGC20461 | NM_020530 | ENSG00000099985 | 5008 |
| | precursor (OSM), | | | | | |
| | ENSG00000099985, | | | | | |
| | OSM | | | | | |
| 452 | Oncostatin M | OSM | MGC20461 | NM_020530 | ENSG00000099985 | 5008 |
| | precursor (OSM), | | | | | |
| | ENSG00000099985, | | | | | |
| | OSM | | | | | |
| 453 | Myosin-18B (Myosin | MYO18B | BK125H2.1 | NM_014550 | ENSG00000133454 | 84700 |
| | XVIIIb), | | | | | |
| | ENSG00000133454, | | | | | |
| | MYO18B | | | | | |
| 454 | Q6ICL0_HUMAN | Q6ICL0_HUMAN | Em: AC006547.7, | | ENSG00000184004 | 150197 |
| | (Predicted | (Predicted | FLJ32575 | | | |
| | UniProt/TrEMBL ID), | UniProt/TrEMBL | | | | |
| | hypothetical protein | ID) | | | | |
| | FLJ3257; | | | | | |
| | ENSG00000184004 | | | | | |
| 455 | OTTHUMG00000030140, | CTA- | bA262A13.C22.5 | No | OTTHUMG00000030140 | No Available |
| | CTA-299D3.6 | 299D3.6 | | Available | | |
| | | (Vega gene | | | | |
| | | ID) | | | | |
| 456 | GALR3; galanin | GALR3 | | NM_003614 | ENSG00000128310 | 8484 |
| | receptor 3; | | | | | |
| | ENSG00000128310 | | | | | |
| 457 | GALR3; galanin | GALR3 | | NM_003614 | ENSG00000128310 | 8484 |
| | receptor 3; | | | | | |
| | ENSG00000128310 | | | | | |
| 458 | IL2RB; interleukin 2 | IL2RB | P70-75 | NM_000878 | ENSG00000100385 | 3560 |
| | receptor, beta; | | | | | |
| | ENSG00000100385 | | | | | |
| 459 | CTA-343C1.3 (Vega | CTA- | bK343C1.C22.3 | No | OTTHUMG00000030151 | No Available |
| | gene ID); Putative | 343C1.3 | | Available | | |
| | Processed transcript; | (Vega gene | | | | |
| | OTTHUMG00000030151 | ID) | | | | |
| 460 | CTA-941F9.6 | CTA- | | No | OTTHUMG00000030231 | No Available |
| | (Vega_gene ID) | 941F9.6 | | Available | | |
| | | (Vega_gene | | | | |
| | | ID) | | | | |
| 461 | CTA-941F9.6 | CTA- | | No | OTTHUMG00000030231 | No Available |
| | (Vega_gene ID) | 941F9.6 | | Available | | |
| | | (Vega_gene | | | | |
| | | ID) | | | | |
| 462 | LL22NC03-121E8.1 | LL22NC03- | cN121E8.C22.1 | No | OTTHUMG00000030676 | No Available |
| | (Vega gene ID); Novel | 121E8.1 | | Available | | |
| | Protein coding; | (Vega gene | | | | |
| | OTTHUMG00000030676 | ID) | | | | |
| 463 | Cytohesin-4, | PSCD4 | CYT4 | NM_013385 | ENSG00000100055 | 27128 |
| | ENSG00000100055, | | | | | |
| | PSCD4 | | | | | |
| 464 | RP4-754E20_A.4 | RP4- | dJ754E20A.C22.4 | No | OTTHUMG00000030716 | No Available |
| | (Vega gene ID); | 754E20_A.4 | | Available | | |
| | Putative Processed | (Vega | | | | |
| | transcript; | gene ID) | | | | |
| | OTTHUMG00000030716 | | | | | |
| 465 | PIB5PA; | PIB5PA | PIPP; | NM_001002837 | ENSG00000185133 | 27124 |
| | phosphatidylinositol | | INPP5; | | | |
| | (4,5) bisphosphate 5- | | MGC129984 | | | |
| | phosphatase, A; | | | | | |
| | ENSG00000185133; | | | | | |
| | embryonic marker | | | | | |
| 466 | no gene associated | | | | | |
| 467 | PLA2G3; | PLA2G3 | GIII- | NM_015715 | ENSG00000100078 | 50487 |
| | ENSG00000100078; | | SPLA2 | | | |
| | phospholipase A2, | | | | | |
| | group III | | | | | |
| 468 | PLA2G3; | PLA2G3 | GIII- | NM_015715 | ENSG00000100078 | 50487 |
| | ENSG00000100078; | | SPLA2 | | | |
| | phospholipase A2, | | | | | |
| | group III | | | | | |
| 469 | DGCR2; DiGeorge | DGCR2 | IDD; | NM_005137 | ENSG00000070413 | 9993 |
| | syndrome critical | | LAN; | | | |
| | region gene 2; | | DGS-C; | | | |
| | ENSG00000070413 | | SEZ-12; | | | |
| | | | KIAA0163; | | | |
| | | | DKFZp686I1730 | | | |
| 470 | TCN2; transcobalamin | TCN2 | TC2; | NM_000355 | ENSG00000185339 | 6948 |
| | II; macrocytic anemia; | | D22S676; | | | |
| | ENSG00000185339 | | D22S750 | | | |
| 471 | IGLL1; | IGLL1 | IGO; 14.1; | NM_020070 | ENSG00000128322 | 3543 |
| | immunoglobulin | | IGL1; | | | |
| | lambda-like | | IGL5; | | | |
| | polypeptide 1; | | IGLL; | | | |
| | ENSG00000128322 | | IGVPB; | | | |
| | | | CD179b; | | | |
| | | | VPREB2; | | | |
| | | | IGLJ14.1 | | | |
| 472 | RP1-29C18.7 (Vega | RP1- | dJ29C18.C22.7 | No | OTTHUMG00000030424 | No Available |
| | gene ID); Novel | 29C18.7 | | Available | | |
| | Processed transcript; | (Vega gene | | | | |
| | OTTHUMG00000030424 | ID) | | | | |
| 473 | IGLC1; | IGLC1 | IGLC; | NG_000002 | ENSG00000100208 | 3537 |
| | immunoglobulin | | MGC40381; | | | |
| | lambda constant 1 | | MGC40425; | | | |
| | (Mcg marker); | | MGC88359; | | | |
| | ENSG00000100208 | | MGC88777; | | | |
| | | | MGC88779; | | | |
| | | | MGC104999; | | | |
| | | | DKFZp667J0810; | | | |
| | | | Constant | | | |
| | | | region of | | | |
| | | | lambda | | | |
| | | | light | | | |
| | | | chains; | | | |
| | | | immunoglobulin | | | |
| | | | lambda | | | |
| | | | constant | | | |
| | | | region 1 | | | |
| | | | (Mcg | | | |
| | | | marker) | | | |
| 474 | APOBEC3B; | APOBEC3B | ARP4; | NM_004900 | ENSG00000179750 | 9582 |
| | apolipoprotein B | | ARCD3; | | | |
| | mRNA editing | | PHRBNL; | | | |
| | enzyme, catalytic | | APOBEC1L; | | | |
| | polypeptide-like 3B; | | FLJ21201; | | | |
| | ENSG00000179750 | | DJ742C19.2 | | | |
| 475 | CRYBB1; crystallin, | CRYBB1 | | NM_001887 | ENSG00000100122 | 1414 |
| | beta B1; | | | | | |
| | ENSG00000100122 | | | | | |
| 476 | CRYBA4; crystallin, | CRYBA4 | | NM_001886 | ENSG00000196431 | 1413 |
| | beta A4; | | | | | |
| | ENSG00000196431 | | | | | |
| 477 | sushi domain | SUSD2 | BK65A6.2, | NM_019601 | ENSG00000099994 | 56241 |
| | containing 2, | | FLJ22778 | | | |
| | ENSG00000099994, | | | | | |
| | SUSD2 | | | | | |
| 478 | sushi domain | SUSD2 | BK65A6.2, | NM_019601 | ENSG00000099994 | 56241 |
| | containing 2, | | FLJ22778 | | | |
| | ENSG00000099994, | | | | | |
| | SUSD2 | | | | | |
| 479 | OTTHUMG00000030870, | CTA- | bK503F6.C22.1 | No | OTTHUMG00000030870 | No Available |
| | Putative Processed | 503F6.1 | | Available | | |
| | transcript, CTA- | (Vega gene | | | | |
| | 503F6.1 | ID) | | | | |
| 480 | embryonic marker, | KB- | Em: AP000577.C22.3 | No | OTTHUMG00000030800 | No Available |
| | OTTHUMG00000030800, | 1323B2.3 | | Available | | |
| | KB-1323B2.3 | (Vega gene | | | | |
| | | ID) | | | | |
| 481 | no gene associated | | | | | |
| 482 | IGLV1-44; | IGLV1-44 | V1-16; | NG_000002 | ENSG00000186751 | 28823 |
| | immunoglobulin | | IGLV144 | | | |
| | lambda variable 1-44; | | | | | |
| | ENSG00000186751 | | | | | |
| 483 | IGLV1-44; | IGLV1-44 | V1-16; | NG_000002 | ENSG00000186751 | 28823 |
| | immunoglobulin | | IGLV144 | | | |
| | lambda variable 1-44; | | | | | |
| | ENSG00000186751 | | | | | |
| 484 | OTTHUMG00000030922, | RP3- | Em: AC002378.C22.2 | No | OTTHUMG00000030922 | No Available |
| | RP3-438O4.2 | 438O4.2 | | Available | | |
| | | (Vega_gene | | | | |
| | | ID) | | | | |
| 485 | OTTHUMG00000030922, | RP3- | Em: AC002378.C22.2 | No | OTTHUMG00000030922 | No Available |
| | RP3-438O4.2 | 438O4.2 | | Available | | |
| | | (Vega_gene | | | | |
| | | ID) | | | | |
| 486 | APOL4; | APOL4 | APOLIV; | NM_030643 | ENSG00000100336 | 80832 |
| | apolipoprotein L, 4; | | APOL-IV | | | |
| | ENSG00000100336 | | | | | |
| 487 | OTTHUMG00000030852, | RP4- | DJ756G23.1, | XM_377720 | OTTHUMG00000030852 | 150356 |
| | RP4- | 756G23.1 | LOC150356 | XP_377720 | | |
| | 756G23.1, novel | (Vega gene | | | | |
| | processed transcript | ID) | | | | |
| 488 | ENSG00000100399, | Q96E60_HUMAN | RP4- | XM_377720 | ENSG00000100399 | 150356 |
| | Q96E60_HUMAN | (Predicted | 756G23.1 | XP_377720 | | |
| | | UniProt/TrEMBL | hypothetical | | | |
| | | ID) | protein | | | |
| | | | BC012882, | | | |
| | | | LOC150356 | | | |
| 489 | Neutrophil cytosol | NCF4 | p40phox | NM_000631 | ENSG00000100365 | 4689 |
| | factor 4 (NCF-4) | | | | | |
| | (Neutrophil NADPH | | | | | |
| | oxidase factor 4) (p40- | | | | | |
| | phox) (p40phox)., | | | | | |
| | ENSG00000100365, | | | | | |
| | NCF4 | | | | | |
| 490 | Neutrophil cytosol | NCF4 | p40phox | NM_000631 | ENSG00000100365 | 4689 |
| | factor 4 (NCF-4) | | | | | |
| | (Neutrophil NADPH | | | | | |
| | oxidase factor 4) (p40- | | | | | |
| | phox) (p40phox)., | | | | | |
| | ENSG00000100365, | | | | | |
| | NCF4 | | | | | |
| 491 | Somatostatin receptor | SSTR3 | | NM_001051 | ENSG00000183473 | 6753 |
| | type 3 (SS3R) (SSR- | | | | | |
| | 28), D | | | | | |
| | ENSG00000183473, | | | | | |
| | SSTR3 | | | | | |
| 492 | Somatostatin receptor | SSTR3 | | NM_001051 | ENSG00000183473 | 6753 |
| | type 3 (SS3R) (SSR- | | | | | |
| | 28), D | | | | | |
| | ENSG00000183473, | | | | | |
| | SSTR3 | | | | | |
| 493 | Bcl-2 interacting killer | BIK | NBK, | NM_001197 | ENSG00000100290 | 638 |
| | (Apoptosis inducer | | BBC1 | | | |
| | NBK) (BP4) (BIP1)., | | | | | |
| | ENSG00000100290, | | | | | |
| | BIK | | | | | |
| 494 | GAS2-like protein 1 | GAS2L1 | GAR22 | NM_006478 | ENSG00000185340 | 10634 |
| | (Growth arrest-specific | | | | | |
| | 2-like 1) (GAS2- | | | | | |
| | related protein on | | | | | |
| | chromosome 22) | | | | | |
| | (GAR22 protein), | | | | | |
| | ENSG00000185340, | | | | | |
| | GAS2L1 | | | | | |
| 495 | RP3-355C18.2 (Vega | RP3- | dJ355C18.C22.2 | No | OTTHUMG00000030072 | No Available |
| | gene ID) | 355C18.2 | | Available | | |
| | | (Vega gene | | | | |
| | | ID) | | | | |
| 496 | SOX10; SRY (sex | SOX10 | DOM; | NM_006941 | ENSG00000100146 | 6663 |
| | determining region Y)- | | WS4; | | | |
| | box 10; | | MGC15649 | | | |
| | ENSG00000100146 | | | | | |
| 497 | Gamma-parvin | PARVG | | NM_022141 | ENSG00000138964 | 64098 |
| | ENSG00000138964 | | | | | |
| 498 | Caspase recruitment | CARD10 | CARMA3, | NM_014550 | ENSG00000100065 | 29775 |
| | domain protein 10 | | BIMP1 | | | |
| | (CARD-containing | | | | | |
| | MAGUK protein 3) | | | | | |
| | (Carma | | | | | |
| | 3). ENSG00000100065, | | | | | |
| | CARD10 | | | | | |
| 499 | ENSG00000100101, | NP_077289.1 | MGC3731; | NM_024313 | ENSG00000100101 | 79159 |
| | NP_077289.1 | | dJ37E16.7 | | | |
| 500 | HTF9C; HpaII tiny | HTF9C_HUMAN | HTF9C; | NM_022727 | ENSG00000099899 | 27037 |
| | fragments locus 9C; | (UniProt/Swiss- | MGC102728 | | | |
| | ENSG00000099899 | Prot | | | | |
| | | ID) | | | | |
| 501 | Oncostatin M | OSM | MGC20461 | NM_020531 | ENSG00000099985 | 5008 |
| | precursor (OSM), | | | | | |
| | ENSG00000099985, | | | | | |
| | OSM | | | | | |
| 502 | CTA-407F11.4 (Vega | CTA- | bK407F11.C22.4 | No | OTTHUMG00000030804 | No Available |
| | gene ID); Novel | 407F11.4 | | Available | | |
| | Processed transcript; | (Vega gene | | | | |
| | OTTHUMG00000030804 | ID) | | | | |
| 503 | Q6ICL0_HUMAN | Q6ICL0_HUMAN | Em: AC006547.7, | | ENSG00000184004 | 150197 |
| | (Predicted | (Predicted | FLJ32575 | | | |
| | UniProt/TrEMBL ID), | UniProt/TrEMBL | | | | |
| | hypothetical protein | ID) | | | | |
| | FLJ3257; | | | | | |
| | ENSG00000184004 | | | | | |
| 504 | CTA-989H11.2 (Vega | CTA- | bK989H11.C22.2 | No | OTTHUMG00000030141 | No Available |
| | gene ID); Putative | 989H11.2 | | Available | | |
| | Processed transcript; | (Vega gene | | | | |
| | OTTHUMG00000030141 | ID) | | | | |
| 505 | transmembrane | TMPRSS6 | | NM_153609 | ENSG00000187045 | 164656 |
| | protease, serine 6 | | | | | |
| 506 | HMG2L1; high- | HMG2L1 | HMGBCG; | NM_001003681 | ENSG00000100281 | 10042 |
| | mobility group protein | | THC211630 | | | |
| | 2-like 1; | | | | | |
| | ENSG00000100281 | | | | | |
| 507 | NP_001017964.1 | NP_001017964.1 | LOC150223; | NM_001017964 | ENSG00000161179 | 150223 |
| | (RefSeq peptide ID); | (RefSeq | MGC133160 | | | |
| | hypothetical protein | peptide ID) | | | | |
| | LOC150223; | | | | | |
| | ENSG00000161179 | | | | | |
| 508 | Platelet-derived | PDGFB | SIS; SSV; | NM_002608 | ENSG00000100311 | 5155 |
| | growth factor B chain | | PDGF2; c- | | | |
| | precursor (PDGF B- | | sis | | | |
| | chain, | | | | | |
| | ENSG00000100311, | | | | | |
| | PDGFB | | | | | |
| 509 | OTTHUMG00000030815, | CTA- | Em: U62317.C22.15 | No | OTTHUMG00000030815 | No Available |
| | CTA-384D8.15 | 384D8.15 | | Available | | |
| | | (Vega gene | | | | |
| | | ID) | | | | |
| 510 | MGAT3; mannosyl | MGAT3 | GNT3; | NM_002409 | ENSG00000128268 | 4248 |
| | (beta-1,4-)- | | GNT-III | | | |
| | glycoprotein beta-1,4- | | | | | |
| | N- | | | | | |
| | acetylglucosaminyltransferase; | | | | | |
| | ENSG00000128268 | | | | | |
| 511 | Ceramide kinase (EC | CERK | LK4; | NM_022766 | ENSG00000100422 | 64781 |
| | 2.7.1.138) | | hCERK; | | | |
| | (Acylsphingosine | | FLJ21430; | | | |
| | kinase) (hCERK) | | FLJ23239; | | | |
| | (Lipid kinase 4) | | KIAA1646; | | | |
| | (LK4), | | MGC131878; | | | |
| | ENSG00000100422, | | dA59H18.2; | | | |
| | CERK | | dA59H18.3; | | | |
| | | | DKFZp434E0211 | | | |
| 512 | Reticulon 4 receptor | RTN4R | NGR; | NM_023004 | ENSG00000040608 | 65078 |
| | precursor (Nogo | | NOGOR | | | |
| | receptor) (NgR) | | | | | |
| | (Nogo-66 receptor), | | | | | |
| | ENSG00000040608, | | | | | |
| | RTN4R | | | | | |
| 513 | UNC84B; unc-84 | UNC84B | SUN2; | NM_015374 | ENSG00000100242 | 25777 |
| | homolog B (C. Elegans); | | KIAA0668 | | | |
| | ENSG00000100242 | | | | | |
| 514 | RABL4; RAB, | RABL4 | RAYL | NM_006860 | ENSG00000100360 | 11020 |
| | member of RAS | | | | | |
| | oncogene family-like | | | | | |
| | 4; ENSG00000100360 | | | | | |
| 515 | Cadherin EGF LAG | CELSR1 | ME2, | NM_014246 | ENSG00000075275 | 9620 |
| | seven-pass G-type | | HFMI2, | | | |
| | receptor 1 precursor | | FMI2, | | | |
| | (Flamingo homolog 2) | | CDHF9 | | | |
| | (hFmi2), | | | | | |
| | ENSG00000075275, | | | | | |
| | CELSR1 | | | | | |
| 516 | OTTHUMG00000030326, | LL22NC03- | cN5H6.C22.1 | No | OTTHUMG00000030326 | No Available |
| | LL22NC03- | 5H6.1 | | Available | | |
| | 5H6.1 | (Vega gene | | | | |
| | | ID) | | | | |
| 517 | OTTHUMG00000030656, | RP3- | Em: AC005005.C22.6 | No | OTTHUMG00000030656 | No Available |
| | RP3-515N1.6 | 515N1.6 | | Available | | |
| | | (Vega gene | | | | |
| | | ID) | | | | |
| 518 | SMTN; smoothelin; | SMTN | | NM_006932 | ENSG00000183963 | 6525 |
| | ENSG00000183963 | | | | | |
| 519 | ZNRF3 protein | ZNRF3 | KIAA1133, | XM_290972, | ENSG00000183579 | 84133 |
| | (Fragment), | | BK747E2.3, | XP_290972 | | |
| | ENSG00000183579, | | FLJ22057, | | | |
| | ZNRF3 zinc and ring | | RNF203 | | | |
| | finger 3 (ZNRF3) | | | | | |
| 520 | OTTHUMG00000030700, | GRAP2 | Grf40, | | OTTHUMG00000030700 | 9402 |
| | GRB2-related | | GrbX, | | | |
| | adaptor protein 2, | | GRBLG, | | | |
| | GRAP2 | | GADS, | | | |
| | | | Mona, | | | |
| | | | P38; | | | |
| | | | GRID; | | | |
| | | | GRPL; | | | |
| | | | GRB2L; | | | |
| | | | GRAP-2 | | | |
| 521 | CAP-binding protein | NP_073622.2 | FLJ23588; | NM_022785 | ENSG00000186976 | 64800 |
| | complex interacting | (RefSeq | DJBP; | | | |
| | protein 1 isoform a | peptide ID) | HSCBCIP1; | | | |
| | Source: | | KIAA1672; | | | |
| | RefSeq_peptide | | dJ185D5.1 | | | |
| | NP_073622 | | | | | |
| 522 | SAM50_HUMAN | SAMM50 | OMP85; | NM_015380 | ENSG00000100347 | 25813 |
| | (UniProt/Swiss-Prot | | SAM50; | | | |
| | ID), | | TOB55; | | | |
| | ENSG00000100347, | | TRG-3; | | | |
| | SAM50-like protein | | CGI-51; | | | |
| | CGI-51; sorting and | | YNL026W | | | |
| | assembly machinery | | | | | |
| | component 50 | | | | | |
| | homolog (S. Cerevisiae) | | | | | |
| 523 | SULT4A1; | SULT4A1 | NST; | NM_014351 | ENSG00000130540 | 25830 |
| | sulfotransferase family | | BRSTL1; | | | |
| | 4A, member 1; | | SULTX3; | | | |
| | ENSG00000130540 | | BR-STL-1; | | | |
| | | | MGC40032; | | | |
| | | | DJ388M5.3; | | | |
| | | | hBR- | | | |
| | | | STL-1 | | | |
| 524 | TIMP3; TIMP | TIMP3 | SFD; | NM_000362 | ENSG00000100234 | 7078 |
| | metallopeptidase | | K222; | | | |
| | inhibitor 3 (Sorsby | | K222TA2; | | | |
| | fundus dystrophy, | | HSMRK222 | | | |
| | pseudoinflammatory); | | | | | |
| | ENSG00000100234 | | | | | |
| 525 | T-box transcription | TBX1 | DGS; | NM_005992 | ENSG00000184058 | 6899 |
| | factor TBX1 (T-box | | TGA; | | | |
| | protein 1) (Testis- | | CAFS; | | | |
| | specific T-box | | CTHM; | | | |
| | protein), | | DGCR; | | | |
| | ENSG00000184058, | | DORV; | | | |
| | TBX1, TBX1 is | | VCFS; | | | |
| | involved in heart | | TBX1C | | | |
| | development-Great | | | | | |
| 526 | ENSG00000186732, | MPPED1 | 239AB; | NM_001585 | ENSG00000186732 | 758 |
| | metallophosphoesterase | | FAM1A; | | | |
| | domain containing 1 | | C22orf1; | | | |
| | NM_001585.2 | | MGC88045 | | | |
| 527 | ENSG00000188511, | NP_942148.1 | LOC348645 | NM_198851 | ENSG00000188511 | 348645 |
| | NP_942148.1 novel | (RefSeq | | | | |
| | Gene hypothetical | peptide ID) | | | | |
| | protein LOC348645 | | | | | |
| 528 | Cdc42 effector protein | CDC42EP1 | MSE55, | NM_007061 | ENSG00000128283 | 11135 |
| | 1, | | CEP1, | | | |
| | ENSG00000128283, | | Borg5, | | | |
| | CDC42EP1 | | MGC15316 | | | |
| 529 | RPL3; ribosomal | RPL3 | TARBP-B; | NM_000967 | ENSG00000100316 | 6122 |
| | protein L3; | | MGC104284 | | | |
| | ENSG00000100316 | | | | | |
| 530 | APOL2; | APOL2 | APOL-II | NM_030882 | ENSG00000128335 | 23780 |
| | apolipoprotein L, 2; | | | | | |
| | ENSG00000128335 | | | | | |
| 531 | RAC2; ras-related C3 | RAC2 | Gx; EN-7; | NM_002872 | ENSG00000128340 | 5880 |
| | botulinum toxin | | HSPC022 | | | |
| | substrate 2 (rho | | | | | |
| | family, small GTP | | | | | |
| | binding protein Rac2); | | | | | |
| | ENSG00000128340 | | | | | |
| 532 | OTTHUMP00000028917, | Q96E60_HUMAN | RP4- | XM_377720 | ENSG00000100399 | 150356 |
| | ENSG00000100399, | (Predicted | 756G23.1 | XP_377720 | | |
| | Q96E60_HUMAN | UniProt/TrEMBL | hypothetical | | | |
| | | ID) | protein | | | |
| | | | BC012882, | | | |
| | | | LOC150356 | | | |
| 533 | Neutrophil cytosol | NCF4 | p40phox | NM_000631 | ENSG00000100365 | 4689 |
| | factor 4 (NCF-4) | | | | | |
| | (Neutrophil NADPH | | | | | |
| | oxidase factor 4) (p40- | | | | | |
| | phox) (p40phox)., | | | | | |
| | ENSG00000100365, | | | | | |
| | NCF4 | | | | | |
| 534 | XP_371837.1 (RefSeq | XP_371837.1 | | No | ENSG00000168768 | No Available |
| | peptide predicted ID); | (RefSeq | | Available | | |
| | PREDICTED: similar | peptide | | | | |
| | to oxidoreductase | predicted | | | | |
| | UCPA Source: | ID) | | | | |
| | RefSeq_peptide_predicted | | | | | |
| | XP_371837; | | | | | |
| | ENSG00000168768 | | | | | |
| 535 | triggering receptor | TREML2 | FLJ13693, | NM_024807 | ENSG00000112195 | 79865 |
| | expressed on myeloid | | TLT2, | | | |
| | cells-like 2, | | dJ238O23.1 | | | |
| | ENSG00000112195, | | | | | |
| | TREML2 | | | | | |
| 536 | TREML1; triggering | TREML1 | TLT1; | NM_178174 | ENSG00000161911 | 340205 |
| | receptor expressed on | | TLT-1; | | | |
| | myeloid cells-like 1; | | PRO3438; | | | |
| | ENSG00000161911 | | GLTL1825; | | | |
| | | | MGC119173; | | | |
| | | | dJ238O23.3 | | | |
| 537 | ENSG00000178199, | ZC3H12D | FLJ46041; | XM_291154 | ENSG00000178199 | 340152 |
| | Q6ZRW2_HUMAN; | | C6orf95; | XP_291154 | | |
| | zinc finger CCCH- | | dJ281H8.1 | | | |
| | type containing 12D | | | | | |
| 538 | AIM1; absent in | AIM1 | ST4 | NM_001624 | ENSG00000112297 | 202 |
| | melanoma1; | | | | | |
| | ENSG00000112297 | | | | | |
| 539 | NKG2D ligand 4 | RAET1E | LETAL, | NM_139165 | ENSG00000164520 | 135250 |
| | precursor (NKG2D | | bA350J20.7, | | | |
| | ligand 4) (NKG2DL4) | | ULBP4; | | | |
| | (N2DL-4) (Retinoic | | MGC125308; | | | |
| | acid early transcript | | MGC125309; | | | |
| | 1E) (Lymphocyte | | bA350J20.7 | | | |
| | effector toxicity | | | | | |
| | activation ligand) | | | | | |
| | (RAE-1-like transcript | | | | | |
| | 4) (RL-4), | | | | | |
| | ENSG00000164520, | | | | | |
| | RAET1E | | | | | |
| 540 | Disheveled associated | DAAM2 | KIAA0381; | NM_015345 | ENSG00000146122 | 23500 |
| | activator of | | MGC90515; | | | |
| | morphogenesis 2, | | dJ90A20A.1; | | | |
| | ENSG00000146122, | | RP1- | | | |
| | DAAM2 | | 278E11.1 | | | |
| 541 | RP11-535K1.1 (Vega | RP11- | | No | OTTHUMG00000014660 | No Available |
| | gene ID); Putative | 535K1.1 | | Available | | |
| | Processed transcript; | (Vega gene | | | | |
| | OTTHUMG00000014660 | ID) | | | | |
| 542 | OTTHUMG00000015679; | RP3- | dJ509I19.3 | No | OTTHUMG00000015679 | No Available |
| | Novel Protein | 509I19.3 | | Available | | |
| | coding; RP3-509I19.3 | (Vega gene | | | | |
| | | ID) | | | | |
| 543 | RP11-503C24.1 (Vega | RP11- | bA503C24.1 | No | OTTHUMG00000016040 | No Available |
| | gene ID); Putative | 503C24.1 | | Available | | |
| | Processed transcript; | (Vega gene | | | | |
| | OTTHUMG00000016040 | ID) | | | | |
| 544 | GABRR2; gamma- | GABRR2 | | NM_002043 | ENSG00000111886 | 2570 |
| | aminobutyric acid | | | | | |
| | (GABA) receptor, rho | | | | | |
| | 2; ENSG00000111886 | | | | | |
| 545 | ANKRD6; ankyrin | ANKRD6 | | NM_014942 | ENSG00000135299 | 22881 |
| | repeat domain 6; | | | | | |
| | ENSG00000135299 | | | | | |
| 546 | TXLNB; taxilin beta; | TXLNB | MDP77; | NM_153235 | ENSG00000164440 | 167838 |
| | ENSG00000164440 | | LST001; | | | |
| | | | C6orf198; | | | |
| | | | dJ522B19.2; | | | |
| | | | DKFZp451A175 | | | |
| 547 | TXLNB; taxilin beta; | TXLNB | MDP77; | NM_153235 | ENSG00000164440 | 167838 |
| | ENSG00000164440 | | LST001; | | | |
| | | | C6orf198; | | | |
| | | | dJ522B19.2; | | | |
| | | | DKFZp451A175 | | | |
| 548 | RP5-899B16.2 (Vega | RP5- | dJ899B16.2 | No | OTTHUMG00000015698 | No Available |
| | gene ID); Putative | 899B16.2 | | Available | | |
| | Processed transcript; | (Vega gene | | | | |
| | OTTHUMG00000015698 | ID) | | | | |
| 549 | Probable G-protein | GPR116 | KPG_001; | NM_015234 | ENSG00000069122 | 221395 |
| | coupled receptor 116 | | KIAA0758; | | | |
| | precursor, | | DKFZp564O1923 | | | |
| | ENSG00000069122, | | | | | |
| | GPR116 | | | | | |
| 550 | RP11-146I2.1 (Vega | RP11- | dJ190J20.1 | No | OTTHUMG00000014290 | No Available |
| | gene ID); Novel | 146I2.1 | | Available | | |
| | Processed transcript; | (Vega gene | | | | |
| | OTTHUMG00000014290 | ID) | | | | |
| 551 | GPR115; G protein- | GPR115 | PGR18; | NM_153838 | ENSG00000153294 | 221393 |
| | coupled receptor 115; | | FLJ38076 | | | |
| | ENSG00000153294 | | | | | |
| 552 | GPR126; G protein- | GPR126 | DREG; | NM_001032394 | ENSG00000112414 | 57211 |
| | coupled receptor 126; | | VIGR; | | | |
| | ENSG00000112414 | | PS1TP2 | | | |
| | embryonic marker | | | | | |
| 553 | RP1-60O19.1 (Vega | RP1- | dJ60O19.1 | No | OTTHUMG00000015305 | No Available |
| | gene ID); Known | 60O19.1 | | Available | | |
| | Processed transcript; | (Vega gene | | | | |
| | OTTHUMG00000015305 | ID) | | | | |
| 554 | OTTHUMG00000015313, | SCML4 | RP1- | NM_198081 | OTTHUMG00000015313 | 256380 |
| | RP1-47M23.1 | | 47M23.1 | | | |
| | SCML4 sex comb on | | (Vega_gene | | | |
| | midleg-like 4 | | ID) | | | |
| | (Drosophila) [Homo | | | | | |
| | sapiens] | | | | | |
| 555 | OTTHUMG00006004170, | CRISP2 | TPX1; | NM_003296 | OTTHUMG00000014822 | 7180 |
| | TPX1testis | | TSP1; | | | |
| | specific protein 1 | | GAPDL5; | | | |
| | (probe H4-1 p3-1) | | CRISP-2; | | | |
| | | | MGC111136 | | | |
| 556 | OTTHUMG00000014829, | RP11- | bA397G17.1 | No | OTTHUMG00000014829 | No Available |
| | RP11-397017.1, novel | 397G17.1 | | Available | | |
| | processed transcript. | (Vega gene | | | | |
| | | ID) | | | | |
| 557 | OTTHUMG00000015337RP11- | RP11- | LOC389422 | XM_374179 | OTTHUMG00000015337 | 389422 |
| | 487F23.3 | 487F23.3 | | XP_374179 | | |
| | hypothetical | (Vega gene | | | | |
| | LOC389422 | ID) | | | | |
| 558 | Nesprin-1 (Nuclear | SYNE1 | SYNE-1B, | NM_015293 | ENSG00000131018 | 23345 |
| | envelope spectrin | | KIAA0796, | | | |
| | repeat protein 1) | | 8B, | | | |
| | (Synaptic nuclear | | nesprin-1, | | | |
| | envelope protein 1) | | enaptin, | | | |
| | (Syne-1) (Myocyte | | MYNE1, | | | |
| | nuclear envelope | | CPG2 | | | |
| | protein 1) (Myne-1) | | | | | |
| | (Enaptin), | | | | | |
| | ENSG00000131018, | | | | | |
| | SYNE1 | | | | | |
| 559 | Nesprin-1 (Nuclear | SYNE1 | SYNE-1B, | NM_015293 | ENSG00000131018 | 23345 |
| | envelope spectrin | | KIAA0796, | | | |
| | repeat protein 1) | | 8B, | | | |
| | (Synaptic nuclear | | nesprin-1, | | | |
| | envelope protein 1) | | enaptin, | | | |
| | (Syne-1) (Myocyte | | MYNE1, | | | |
| | nuclear envelope | | CPG2 | | | |
| | protein 1) (Myne-1) | | | | | |
| | (Enaptin), | | | | | |
| | ENSG00000131018, | | | | | |
| | SYNE1 | | | | | |
| 560 | RP11-398K22.4 (Vega | RP11- | Em: AC019205.4 | No | OTTHUMG0000001504 | No Available |
| | gene ID); Putative | 398K22.4 | | Available | | |
| | Processed transcript; | (Vega gene | | | | |
| | OTTHUMG00000015024 | ID) | | | | |
| 561 | MyoD family inhibitor | MDFI | I-MF | NM_005586 | ENSG00000112559 | 4188 |
| | (Myogenic repressor I- | | | | | |
| | mf), | | | | | |
| | ENSG00000112559, | | | | | |
| | MDFI | | | | | |
| 562 | OTTHUMG00000014691, | RP11- | bA533O20.2 | No | OTTHUMG00000014691 | No Available |
| | putative | 533O20.2 | | Available | | |
| | processed transcript, | (Vega gene | | | | |
| | RP11-533O20.2 | ID) | | | | |
| 563 | RP3-398D13.4 (Vega | RP3- | dJ398D13.4 | No | OTTHUMG00000014188 | No Available |
| | gene ID); | 398D13.4 | | Available | | |
| | OTTHUMG00000014188 | (Vega gene | | | | |
| | | ID); | | | | |
| 564 | RP3-429O6.1 (Vega | RP3- | dJ429O6.1 | No | OTTHUMG00000014195 | No Available |
| | gene ID); Putative | 429O6.1 | | Available | | |
| | Processed transcript; | (Vega gene | | | | |
| | OTTHUMG00000014195 | ID) | | | | |
| 565 | MOG; myelin | MOG | MGC26137 | NM_001008228 | ENSG00000137345 | 4340 |
| | oligodendrocyte | | | | | |
| | glycoprotein; | | | | | |
| | ENSG00000137345 | | | | | |
| 566 | RP3-495K2.2 (Vega | RP3- | dJ495K2.2 | No | OTTHUMG00000016052 | No Available |
| | gene ID); Putative | 495K2.2 | | Available | | |
| | Processed transcript; | (Vega gene | | | | |
| | OTTHUMG00000016052 | ID) | | | | |
| 567 | RP11-417E7.1 (Vega | RP11- | bA417E7.1 | No | OTTHUMG00000016054 | No Available |
| | gene ID); Putative | 417E7.1 | | Available | | |
| | Processed transcript; | (Vega gene | | | | |
| | OTTHUMG00000016054 | ID) | | | | |
| 568 | tyrosine-protein | PTK7 | CCK4 | NM_002821 | ENSG00000112655 | 5754 |
| | kinase-like 7 precursor | | | | | |
| | (Colon carcinoma | | | | | |
| | kinase 4) (CCK-4)., | | | | | |
| | ENSG00000112655, | | | | | |
| | PTK7 | | | | | |
| 569 | RP11-174C7.4 (Vega | RP11- | bA174C7.4 | No | OTTHUMG00000015553 | No Available |
| | gene ID) | 174C7.4 | | Available | | |
| | | (Vega gene | | | | |
| | | ID) | | | | |
| 570 | cytidine | CMAH | CSAH; | NR_002174 | OTTHUMG00000016099 | 8418 |
| | monophosphate-N- | | CMP- | | | |
| | acetylneuraminic acid | | NeuAc | | | |
| | hydroxylase (CMP-N- | | hydroxylase; | | | |
| | acetylneuraminate | | CMP- | | | |
| | monooxygenase); | | Neu5Ac | | | |
| | CMAH | | hydroxylase; | | | |
| | | | CMP- | | | |
| | | | sialic acid | | | |
| | | | hydroxylase; | | | |
| | | | CMP-N- | | | |
| | | | acetylneuraminic | | | |
| | | | acid | | | |
| | | | hydroxylase; | | | |
| | | | cytidine | | | |
| | | | monophos | | | |
| | | | pho-N- | | | |
| | | | acetylneuraminic | | | |
| | | | acid | | | |
| | | | hydroxylase | | | |
| 571 | KHD1; polycystic | PKHD1 | FCYT; | NM_138694 | ENSG00000170927 | 5314 |
| | kidney and hepatic | | ARPKD; | | | |
| | disease 1 (autosomal | | TIGM1 | | | |
| | recessive); | | | | | |
| | ENSG00000170927 | | | | | |
| 572 | RP3-471C18.2 (Vega | RP3- | dJ471C18.2 | No | OTTHUMG00000014332 | No Available |
| | gene ID); Novel | 471C18.2 | | Available | | |
| | Processed transcript; | (Vega gene | | | | |
| | OTTHUMG00000014332 | ID) | | | | |
| 573 | RP11-204E9.1 (Vega | RP11- | bA204E9.1 | No | OTTHUMG00000014342 | No Available |
| | gene ID); Putative | 204E9.1 | | Available | | |
| | Processed transcript; | (Vega gene | | | | |
| | OTTHUMG00000014342 | ID) | | | | |
| 574 | glutathione peroxidase | GPX5 | | NM_001509 | OTTHUMG00000016307 | 2880 |
| | 5, | | | | | |
| | OTTHUMG00000016307, | | | | | |
| | GPX5 | | | | | |
| 575 | RP11-411K7.1 (Vega | RP11- | bA411K7.1 | No | OTTHUMG00000014887 | No Available |
| | gene ID); Putative | 411K7.1 | | Available | | |
| | Processed transcript; | (Vega gene | | | | |
| | OTTHUMG00000014887 | ID) | | | | |
| 576 | skin marker, | GRIK2 | RP3- | NM_021956 | ENSG00000164418 | 2898 |
| | Glutamate receptor, | | 438O4.2 | | | |
| | ionotropic kainate 2 | | (Vega gene | | | |
| | precursor (Glutamate | | ID) | | | |
| | receptor 6) (GluR-6) | | | | | |
| | (GluR6) (Excitatory | | | | | |
| | amino acid receptor 4) | | | | | |
| | (EAA4) | | | | | |
| 577 | C6orf142; | C6orf142 | MGC18257 | NM_138569 | ENSG00000146147 | 90523 |
| | chromosome 6 open | | | | | |
| | reading frame 142; | | | | | |
| | ENSG00000146147 | | | | | |
| 578 | HDGFL1; hepatoma | HDGFL1 | PWWP1; | NM_138574 | ENSG00000112273 | 154150 |
| | derived growth factor- | | dJ309H15.1 | | | |
| | like 1; | | | | | |
| | ENSG00000112273 | | | | | |
| 579 | forkhead box C1, | FOXC1 | FKHL7, | NM_001453 | OTTHUMG00000016182 | 2296 |
| | OTTHUMG00000016182, | | IRID1, | | | |
| | FOXC1 | | FREAC3, | | | |
| | | | ARA, | | | |
| | | | IGDA, | | | |
| | | | IHG1 | | | |
| 580 | C6orf188; | C6orf188 | MGC45451; | NM_153711 | ENSG00000178033 | 254228 |
| | chromosome 6 open | | dJ493F7.3 | | | |
| | reading frame 188; | | | | | |
| | ENSG00000178033 | | | | | |
| 581 | ME1; malic enzyme 1, | ME1 | MES; | NM_002395 | ENSG00000065833 | 4199 |
| | NADP(+)-dependent, | | HUMNDME | | | |
| | cytosolic; | | | | | |
| | ENSG00000065833 | | | | | |
| 582 | SLC22A1; solute | SLC22A1 | OCT1; | NM_003057 | ENSG00000175003 | 6580 |
| | carrier family 22 | | | | | |
| | (organic cation | | | | | |
| | transporter), member 1 | | | | | |
| 583 | RP11-235G24.1 (Vega | RP11- | bA235G24.1 | No | OTTHUMG00000015959 | No Available |
| | gene ID) | 235G24.1 | | Available | | |
| | | (Vega gene | | | | |
| | | ID) | | | | |
| 584 | T-box 18; TBX18 | TBX18 | | XM_496819 | ENSG00000112837 | 9096 |
| | | | | XP_496819 | | |
| 585 | CTA-31J9.2, putative | CTA- | bK31J9.2 | No | OTTHUMG00000015619 | No Available |
| | processed transcript, | 31J9.2 | | Available | | |
| | OTTHUMG00000015619 | (Vega gene | | | | |
| | | ID) | | | | |
| 586 | RP1-32B1.4 (Vega | RP1- | dJ32B1.4 | No | OTTHUMG00000015628 | No Available |
| | gene ID); Putative | 32B1.4 | | Available | | |
| | Processed transcript | (Vega gene | | | | |
| | OTTHUMG00000015628 | ID) | | | | |
| 587 | OTTHUMG00000014223, | RP11- | bA203H2.2 | No | OTTHUMG00000014223 | No Available |
| | RP11-203H2.2, | 203H2.2 | | Available | | |
| | novel processed | (Vega gene | | | | |
| | treanscript | ID) | | | | |
| 588 | OTTHUMG00000014737, | C6orf154 | MGC131686; | NM_001012974 | OTTHUMG00000014737 | 221424 |
| | C6orf154 and | | dJ337H4.2 | | | |
| | Name: chromosome 6 | | | | | |
| | open reading frame | | | | | |
| | 154; RP3-337H4.2 | | | | | |
| 589 | transcription factor | TFAP2A | AP-2 | NM_001032280 | OTTHUMG00000014235 | 7020 |
| | AP-2 alpha, | | | | | |
| | OTTHUMG00000014235, | | | | | |
| | TFAP2A | | | | | |
| 590 | IL20RA; interleukin | IL20RA | IL-20R1; | NM_014432 | ENSG00000016402 | 53832 |
| | 20 receptor, alpha; | | ZCYTOR7 | | | |
| | ENSG00000016402 | | | | | |
| 591 | KAAG1; kidney | KAAG1 | RU2; | NM_181337 | ENSG00000146049 | 353219 |
| | associated antigen 1; | | RU2AS; | | | |
| | ENSG00000146049 | | MGC78738 | | | |
| 592 | TGM3; | TGM3 | TGE; | NM_003245 | ENSG00000125780 | 7053 |
| | transglutaminase 3 (E | | MGC126249; | | | |
| | polypeptide, protein- | | MGC126250 | | | |
| | glutamine-gamma- | | | | | |
| | glutamyltransferase); | | | | | |
| | ENSG00000125780 | | | | | |
| 593 | RASSF2; Ras | RASSF2 | KIAA0168; | NM_014737 | ENSG00000101265 | 9770 |
| | association | | DKFZp781O1747 | | | |
| | (RalGDS/AF-6) | | | | | |
| | domain family 2; | | | | | |
| | ENSG00000101265 | | | | | |
| 594 | no gene associated | | | | | |
| 595 | no gene associated | | | | | |
| 596 | no gene associated | | | | | |
| 597 | no gene associated | | | | | |
| 598 | no gene associated | | | | | |
| 599 | no gene associated | | | | | |
| 600 | no gene associated | | | | | |
| 601 | no gene associated | | | | | |
| 602 | no gene associated | | | | | |
| 603 | no gene associated | | | | | |
| 604 | no gene associated | | | | | |
| 605 | RP4-697P8.2 (Vega | RP4- | dJ697P8.2 | No | OTTHUMG00000031879 | No Available |
| | gene ID); Putative | 697P8.2 | | Available | | |
| | Processed transcript; | (Vega gene | | | | |
| | OTTHUMG00000031879 | ID) | | | | |
| 606 | no gene associated | | | | | |
| 607 | OTTHUMG00000031883, | RP4- | DJ734C18.1 | No | OTTHUMG00000031883 | No Available |
| | RP4-734C18.1, | 734C18.1 | | Available | | |
| | putative processed | (Vega gene | | | | |
| | transcript | ID) | | | | |
| 608 | no gene associated | | | | | |
| 609 | no gene associated | | | | | |
| 610 | no gene associated | | | | | |
| 611 | no gene associated | | | | | |
| 612 | no gene associated | | | | | |
| 613 | Ras and Rab interactor | RIN2 | RASSF4 | NM_018993 | OTTHUMG00000031996 | 54453 |
| | 2, | | | | | |
| | OTTHUMG00000031996, | | | | | |
| | RIN2 | | | | | |
| 614 | no gene associated | | | | | |
| 615 | no gene associated | | | | | |
| 616 | no gene associated | | | | | |
| 617 | no gene associated | | | | | |
| 618 | no gene associated | | | | | |
| 619 | no gene associated | | | | | |
| 620 | no gene associated | | | | | |
| 621 | no gene associated | | | | | |
| 622 | no gene associated | | | | | |
| 623 | no gene associated | | | | | |
| 624 | C20orf112; | C20orf112 | dJ1184F4.2; | NM_080616 | OTTHUMG00000032219 | 140688 |
| | chromosome 20 open | | DKFZP566G1424 | | | |
| | reading frame 112; | | | | | |
| | OTTHUMG00000032219 | | | | | |
| 625 | FER1L4; fer-1-like 4 | FER1L4 | FLJ13459; | NR_001442 | OTTHUMG00000032354 | 80307 |
| | (C. Elegans); | | FLJ22613; | | | |
| | OTTHUMG00000032354 | | C20orf124; | | | |
| | | | dJ309K20.1; | | | |
| | | | bA563A22B.1; | | | |
| | | | fer-1 | | | |
| | | | (C. elegans)- | | | |
| | | | like 4 | | | |
| 626 | no gene associated | | | | | |
| 627 | no gene associated | | | | | |
| 628 | Protein C20orf102 | | | | | |
| | precursor, | | | | | |
| | ENSG00000132821, | | | | | |
| | CT102_HUMAN | | | | | |
| 629 | no gene associated | | | | | |
| 630 | no gene associated | | | | | |
| 631 | no gene associated | | | | | |
| 632 | no gene associated | | | | | |
| 633 | no gene associated - | | | | | |
| | Nearest transcript | | | | | |
| | CDH22 (~18 kb | | | | | |
| | upstream) | | | | | |
| 634 | no gene associated | | | | | |
| 635 | no gene associated | | | | | |
| 636 | no gene associated | | | | | |
| 637 | no gene associated | | | | | |
| 638 | no gene associated | | | | | |
| 639 | no gene associated | | | | | |
| 640 | no gene associated | | | | | |
| 641 | ZHX3; zinc fingers | ZHX3 | TIX1; | NM_015035 | OTTHUMG00000032481 | 23051 |
| | and homeoboxes 3; | | KIAA0395 | | | |
| | OTTHUMG00000032481 | | | | | |
| 642 | no gene associated | | | | | |
| 643 | CHD6; chromodomain | CHD6 | CHD5; | NM_032221 | ENSG00000124177 | 84181 |
| | helicase DNA binding | | RIGB; | | | |
| | protein 6; | | KIAA1335 | | | |
| | ENSG00000124177 | | | | | |
| 644 | no gene associated | | | | | |
| 645 | PTPRG; protein | PTPRG | PTPG; | NM_002841 | ENSG00000144724 | 5793 |
| | tyrosine phosphatase, | | HPTPG; | | | |
| | receptor type, G; | | RPTPG; | | | |
| | ENSG00000144724 | | R-PTP- | | | |
| | Gene is located on | | GAMMA | | | |
| | Chr. 3 | | | | | |
| 646 | no gene associated | | | | | |
| 647 | no gene associated | | | | | |
| 648 | no gene associated | | | | | |
| 649 | PTPNS1; protein | PTPNS1 | BIT; MFR; | NM_080792 | ENSG00000198053 | 140885 |
| | tyrosine phosphatase, | | P84; SIRP; | | | |
| | non-receptor type | | MYD-1; | | | |
| | substrate 1; | | SHPS1; | | | |
| | ENSG00000198053 | | SHPS-1; | | | |
| | | | SIRPalpha; | | | |
| | | | SIRPalpha | | | |
| | | | 2; SIRP- | | | |
| | | | ALPHA-1 | | | |
| 650 | Q7Z5T1_HUMAN | Q7Z5T1_HUMAN | KIAA1442; | XM_044921 | ENSG00000088881 | 57593 |
| | (Predicted | (Predicted | EBF4; | XP_044921 | | |
| | UniProt/TrEMBL ID); | UniProt/TrEMBL | O/E-4; | | | |
| | KIAA1442 protein; | ID) | RP5- | | | |
| | ENSG00000088881 | | 860F19.3 | | | |
| 651 | NP_689717.2 (RefSeq | NP_689717.2 | | No | ENSG00000171984 | No Available |
| | peptide ID); | (RefSeq | | Available | | |
| | ENSG00000171984 | peptide ID) | | | | |
| 652 | ENSG00000149346, | C20orf94 | NP_001009608.1, | NM_001009608 | ENSG00000149346 | 128710 |
| | NP_001009608.1, | | bA204H22.1; | | | |
| | hypothetical protein | | bA254M13.1; | | | |
| | LOC128710, | | dJ1099D15.3 | | | |
| | chromosome 20 open | | | | | |
| | reading frame 94 | | | | | |
| 653 | C20orf82; | C20orf82 | bA149I18.1; | XM_097736 | ENSG00000101230 | 140862 |
| | chromosome 20 open | | dJ1077I2.1 | XP_097736 | | |
| | reading frame 82; | | | | | |
| | ENSG00000101230 | | | | | |
| 654 | C20orf23; | C20orf23 | SNX23; | NM_024704 | ENSG00000089177 | 55614 |
| | chromosome 20 open | | KISC20ORF | | | |
| | reading frame 23; | | | | | |
| | ENSG00000089177; | | | | | |
| | embryonic marker | | | | | |
| 655 | PCSK2; proprotein | PCSK2 | PC2; | NM_002594 | ENSG00000125851 | 5126 |
| | convertase | | NEC2; | | | |
| | subtilisin/kexin type 2; | | SPC2 | | | |
| | ENSG00000125851 | | | | | |
| 656 | PCSK2; proprotein | PCSK2 | PC2; | NM_002594 | ENSG00000125851 | 5126 |
| | convertase | | NEC2; | | | |
| | subtilisin/kexin type 2; | | SPC2 | | | |
| | ENSG00000125851 | | | | | |
| 657 | solute carrier family | SLC24A3 | NCKX3 | NM_020689 | OTTHUMG00000031993 | 57419 |
| | 24 | | | | | |
| | (sodiumVpotassiumVcalcium | | | | | |
| | exchanger), | | | | | |
| | member 3, | | | | | |
| | OTTHUMG00000031993, | | | | | |
| | SLC24A3 ( | | | | | |
| 658 | solute carrier family | SLC24A3 | NCKX3 | NM_020689 | OTTHUMG00000031993 | 57419 |
| | 24 | | | | | |
| | (sodiumVpotassiumVcalcium | | | | | |
| | exchanger), | | | | | |
| | member 3, | | | | | |
| | OTTHUMG00000031993, | | | | | |
| | SLC24A3 ( | | | | | |
| 659 | ENSG00000089101, | C20orf26 | dJ1178H5.4; | NM_015585 | ENSG00000089101 | 26074 |
| | CT026_HUMAN | | DKFZP434K156 | | | |
| 660 | ENSG00000089101, | C20orf26 | dJ1178H5.4; | NM_015585 | ENSG00000089101 | 26074 |
| | CT026_HUMAN | | DKFZP434K156 | | | |
| 661 | C20orf74 protein, | Q9ULE8_HUMAN | FLJ12819; | XM_046600 | ENSG00000188559 | 57186 |
| | ENSG00000188559, | (Predicted | KIAA1272; | XP_046600 | | |
| | Q9ULE8_HUMAN | UniProt/TrEMBL | bA287B20.1; | | | |
| | | ID) | dJ1049G11.4; | | | |
| | | | RP11- | | | |
| | | | 470C13.2 | | | |
| 662 | C20orf74 protein, | Q9ULE8_HUMAN | FLJ12819; | XM_046600 | ENSG00000188559 | 57186 |
| | ENSG00000188559, | (Predicted | KIAA1272; | XP_046600 | | |
| | Q9ULE8_HUMAN | UniProt/TrEMBL | bA287B20.1; | | | |
| | | ID) | dJ1049G11.4; | | | |
| | | | RP11- | | | |
| | | | 470C13.2 | | | |
| 663 | C20orf74 protein, | Q9ULE8_HUMAN | FLJ12819; | XM_046600 | ENSG00000188559 | 57186 |
| | ENSG00000188559, | (Predicted | KIAA1272; | XP_046600 | | |
| | Q9ULE8_HUMAN | UniProt/TrEMBL | bA287B20.1; | | | |
| | | ID) | dJ1049G11.4; | | | |
| | | | RP11- | | | |
| | | | 470C13.2 | | | |
| 664 | PLAGL2; | PLAGL2 | | NM_002657 | ENSG00000126003 | 5326 |
| | pleiomorphic adenoma | | | | | |
| | gene-like 2; | | | | | |
| | ENSG00000126003 | | | | | |
| 665 | GGTL3; gamma- | GGTL3 | GGTL5; | NM_052830 | ENSG00000131067 | 2686 |
| | glutamyltransferase- | | D20S101; | | | |
| | like 3; | | dJ18C9.2 | | | |
| | ENSG00000131067 | | | | | |
| 666 | MYH7B; myosin, | MYH7B | MYH14; | NM_020884 | ENSG00000078814 | 57644 |
| | heavy polypeptide 7B, | | MGC96928; | | | |
| | cardiac muscle, beta; | | MGC96940 | | | |
| | ENSG00000078814 | | | | | |
| 667 | TRPC4AP; transient | TRPC4AP | TRUSS; | NM_015638 | ENSG00000100991 | 26133 |
| | receptor potential | | TRRP4AP; | | | |
| | cation channel, | | C20orf188 | | | |
| | subfamily C, member | | | | | |
| | 4 associated protein; | | | | | |
| | ENSG00000100991 | | | | | |
| 668 | EPB41L1; erythrocyte | EPB41L1 | 4.1N; | NM_012156 | ENSG00000088367 | 2036 |
| | membrane protein | | KIAA0338; | | | |
| | band 4.1-like 1; | | MGC11072 | | | |
| | ENSG00000088367 | | | | | |
| 669 | C20orf117; | C20orf117 | FLJ44670; | NM_080627 | OTTHUMG00000032395 | 140710 |
| | chromosome 20 open | | KIAA0889; | | | |
| | reading frame 117; | | dJ132F21.1 | | | |
| | OTTHUMG00000032395 | | | | | |
| 670 | PTPRT; protein | PTPRT | RPTPrho; | NM_007050 | ENSG00000196090 | 11122 |
| | tyrosine phosphatase, | | KIAA0283 | | | |
| | receptor type, T; | | | | | |
| | ENSG00000196090 | | | | | |
| 671 | PTPRT; protein | PTPRT | RPTPrho; | NM_007050 | ENSG00000196090 | 11122 |
| | tyrosine phosphatase, | | KIAA0283 | | | |
| | receptor type, T; | | | | | |
| | ENSG00000196090 | | | | | |
| 672 | PTPRT; protein | PTPRT | RPTPrho; | NM_007050 | ENSG00000196090 | 11122 |
| | tyrosine phosphatase, | | KIAA0283 | | | |
| | receptor type, T; | | | | | |
| | ENSG00000196090 | | | | | |
| 673 | PTPRT; protein | PTPRT | RPTPrho; | NM_007050 | ENSG00000196090 | 11122 |
| | tyrosine phosphatase, | | KIAA0283 | | | |
| | receptor type, T; | | | | | |
| | ENSG00000196090 | | | | | |
| 674 | PTPRT; protein | PTPRT | RPTPrho; | NM_007050 | ENSG00000196090 | 11122 |
| | tyrosine phosphatase, | | KIAA0283 | | | |
| | receptor type, T; | | | | | |
| | ENSG00000196090 | | | | | |
| 675 | SDC4; syndecan 4 | SDC4 | SYND4; | NM_002999 | ENSG00000124145 | 6385 |
| | (amphiglycan, | | MGC22217 | | | |
| | ryudocan); | | | | | |
| | ENSG00000124145 | | | | | |
| 676 | SDC4; syndecan 4 | SDC4 | SYND4; | NM_002999 | ENSG00000124145 | 6385 |
| | (amphiglycan, | | MGC22217 | | | |
| | ryudocan); | | | | | |
| | ENSG00000124145 | | | | | |
| 677 | cadherin-like 22, | CDH22 | C20orf25; | NM_021248 | OTTHUMG00000033073 | 64405 |
| | OTTHUMG00000033073, | | dJ998H6.1; | | | |
| | CDH22 | | MGC39564 | | | |
| 678 | EYA2; eyes absent | EYA2 | EAB1; | NM_005244 | ENSG00000064655 | 2139 |
| | homolog 2 | | MGC10614 | | | |
| | (Drosophila); | | | | | |
| | ENSG00000064655 | | | | | |
| 679 | SULF2; sulfatase 2; | SULF2 | HSULF-2; | NM_018837 | ENSG00000196562 | 55959 |
| | ENSG00000196562 | | KIAA1247; | | | |
| | | | MGC126411; | | | |
| | | | DKFZp313E091; | | | |
| | | | RP5- | | | |
| | | | 1049G16.1 | | | |
| 680 | KCNB1; potassium | KCNB1 | DRK1; | NM_004975 | ENSG00000158445 | 3745 |
| | voltage-gated channel, | | KV2.1; h- | | | |
| | Shab-related | | DRK1 | | | |
| | subfamily, member 1; | | | | | |
| | ENSG00000158445 | | | | | |
| 681 | Breast carcinoma | BCAS4 | FLJ20495, | NM_001010974 | ENSG00000124243 | 55653 |
| | amplified sequence 4, | | BHLHB4 | | | |
| | ENSG00000124243, | | | | | |
| | BCAS4 | | | | | |
| 682 | nuclear factor of | NFATC2 | NF-ATP, | | OTTHUMG00000032747 | 4773 |
| | activated T-cells, | | NFATp | | | |
| | cytoplasmic, | | | | | |
| | calcineurin-dependent | | | | | |
| | 2, | | | | | |
| | OTTHUMG00000032747, | | | | | |
| | NFATC2 | | | | | |
| 683 | Nuclear factor of | NFATC2 | NF-ATP, | NM_012340 | ENSG00000101096 | 4773 |
| | activated T-cells, | | NFATp | | | |
| | cytoplasmic 2 (T cell | | | | | |
| | transcription factor | | | | | |
| | NFAT1) (NFAT pre- | | | | | |
| | existing subunit) (NF- | | | | | |
| | ATp), | | | | | |
| | ENSG00000101096, | | | | | |
| | NFATC2 | | | | | |
| 684 | Bone morphogenetic | BMP7 | OP-1 | NM_001719 | ENSG00000101144 | 655 |
| | protein 7 precursor | | | | | |
| | (BMP-7) (Osteogenic | | | | | |
| | protein 1) (OP-1) | | | | | |
| | (Eptotermin alfa), | | | | | |
| | ENSG00000101144, | | | | | |
| | BMP7 | | | | | |
| 685 | transmembrane, | TMEPAI | STAG1; | NM_020182 | OTTHUMG00000032831 | 56937 |
| | prostate androgen | | PMEPA1 | | | |
| | induced RNA, | | | | | |
| | OTTHUMG00000032831, | | | | | |
| | TMEPAI | | | | | |
| 686 | ENSG00000176659, | NP_775915.1 | | No | ENSG00000176659 | No Available |
| | NP_775915.1 | (RefSeq | | Available | | |
| | | peptide ID) | | | | |
| 687 | CDH4; cadherin 4, | CDH4 | CAD4; | NM_001794 | ENSG00000179242 | 1002 |
| | type 1, R-cadherin | | RCAD; | | | |
| | (retinal); | | FLJ22202; | | | |
| | ENSG00000179242 | | FLJ40547; | | | |
| | | | MGC126700 | | | |
| 688 | NP_001002034.1 | NP_001002034.1 | | No | ENSG00000177096 | No Available |
| | (RefSeq peptide ID); | (RefSeq | | Available | | |
| | ENSG00000177096 | peptide ID) | | | | |
| 689 | NP_612444.1 (RefSeq | NP_612444.1 | | No | ENSG00000133477 | No Available |
| | peptide ID); | (RefSeq | | Available | | |
| | ENSG00000133477 | peptide ID) | | | | |
| 690 | no gene associated | | | | | |
| 691 | OTTHUMG00000030780, | CTA- | bK373H7.C22.4 | No | OTTHUMG00000030780 | No Available |
| | CTA-373H7.4, novel | 373H7.4 | | Available | | |
| | pseudogene | (Vega gene | | | | |
| | | ID) | | | | |
| 692 | no gene associated | | | | | |
| 693 | Cat eye syndrome | CECR1 | IDGFL | NM_017424 | ENSG00000093072 | 51816 |
| | critical region protein | | | | | |
| | 1 precursor, | | | | | |
| | ENSG00000093072, | | | | | |
| | CECR1 | | | | | |
| 694 | IGLC1; | IGLC1 | IGLC; | NG_000002 | ENSG00000100208 | 3537 |
| | immunoglobulin | | MGC40381; | | | |
| | lambda constant 1 | | MGC40425; | | | |
| | (Mcg marker); | | MGC88359; | | | |
| | ENSG00000100208 | | MGC88777; | | | |
| | | | MGC88779; | | | |
| | | | MGC104999; | | | |
| | | | DKFZp667J0810; | | | |
| | | | Constant | | | |
| | | | region of | | | |
| | | | lambda | | | |
| | | | light | | | |
| | | | chains; | | | |
| | | | immunoglobulin | | | |
| | | | lambda | | | |
| | | | constant | | | |
| | | | region 1 | | | |
| | | | (Mcg | | | |
| | | | marker) | | | |
| 695 | OTTHUMG00000030521, | AC000095.4 | Em: AC000095.C22.4 | No | OTTHUMG00000030521 | No Available |
| | AC000095.4 | (Vega | | Available | | |
| | putative processed | gene ID) | | | | |
| | transcript; | | | | | |
| 696 | Uroplakin-3A | UPK3A | UPK3; | NM_006953 | ENSG00000100373 | 7380 |
| | precursor (Uroplakin | | UPIII | | | |
| | III) (UPIII)., | | | | | |
| | ENSG00000100373, | | | | | |
| | UPK3A | | | | | |
| 697 | Spl site_no gene | | | | | |
| | associated | | | | | |
| 698 | USP18; ubiquitin | USP18 | ISG43; | NM_017414 | OTTHUMG00000030949 | 11274 |
| | specific peptidase 18; | | UBP43 | | | |
| | OTTHUMG00000030949 | | | | | |
| 699 | BCR; breakpoint | BCR | ALL; | NM_004327 | ENSG00000186716 | 613 |
| | cluster region; | | CML; | | | |
| | ENSG00000186716 | | PHL; | | | |
| | | | BCR1; | | | |
| | | | D22S11; | | | |
| | | | D22S662 | | | |
| 700 | TBC1D10A; TBC1 | TBC1D10A | EPI64; | NM_031937 | ENSG00000099992 | 83874 |
| | domain family, | | TBC1D10; | | | |
| | member 10A; | | dJ130H16.1; | | | |
| | ENSG00000099992 | | AC004997.C22.2 | | | |
| 701 | signal peptide-CUB | SCUBE1 | | NM_173050 | ENSG00000159307 | 80274 |
| | domian-EGF-related 1, | | | | | |
| | ENSG00000159307, | | | | | |
| | SCUBE1 | | | | | |
| 702 | MAPK8IP2; mitogen- | MAPK8IP2 | IB2; JIP2; | NM_012324 | ENSG00000008735 | 23542 |
| | activated protein | | PRKM8IPL | | | |
| | kinase 8 interacting | | | | | |
| | protein 2; | | | | | |
| | ENSG00000008735 | | | | | |
| 703 | ENSG00000192797, | No | | NM_000407 | ENSG00000192797 | No Available |
| | miRNA | Available | | | | |
| 704 | RPL3; ribosomal | RPL3 | TARBP-B; | NM_000967 | ENSG00000100316 | 6122 |
| | protein L3; | | MGC104284 | | | |
| | ENSG00000100316 | | | | | |
| 705 | RPL3; ribosomal | RPL3 | TARBP-B; | NM_000967 | ENSG00000100316 | 6122 |
| | protein L3; | | MGC104284 | | | |
| | ENSG00000100316 | | | | | |
| 706 | RP4-695O20_B.9 | RP4- | dJ695O20B.C22.9 | No | OTTHUMG00000030111 | No Available |
| | (Vega gene ID); | 695O20_B.9 | | Available | | |
| | Putative Processed | (Vega | | | | |
| | transcript; | gene ID) | | | | |
| | OTTHUMG00000030111 | | | | | |
| 707 | NOVEL transcript?? | | | | | |
| | No associated gene | | | | | |
| 708 | MN1; meningioma | MN1 | MGCR; | NM_002430 | ENSG00000169184 | 4330 |
| | (disrupted in balanced | | MGCR1; | | | |
| | translocation) 1; | | MGCR1- | | | |
| | ENSG00000169184 | | PEN; | | | |
| | | | dJ353E16.2 | | | |
| 709 | no gene associated | | | | | |
| 710 | RTDR1; rhabdoid | RTDR1 | MGC16968 | NM_014433 | ENSG00000100218 | 27156 |
| | tumor deletion region | | | | | |
| | gene 1; | | | | | |
| | ENSG00000100218 | | | | | |
| 711 | RPL3; ribosomal | RPL3 | TARBP-B; | NM_000967 | ENSG00000100316 | 6122 |
| | protein L3; | | MGC104284 | | | |
| | ENSG00000100316 | | | | | |
| 712 | embryonic marker, | GRAP2 | Grf40, | | OTTHUMG00000030700 | 9402 |
| | GRB2-related adaptor | | GrbX, | | | |
| | protein 2, | | GRBLG, | | | |
| | OTTHUMG00000030700, | | GADS, | | | |
| | GRAP2 | | Mona, | | | |
| | | | P38; | | | |
| | | | GRID; | | | |
| | | | GRPL; | | | |
| | | | GRB2L; | | | |
| | | | GRAP-2 | | | |
| 713 | Serine/threonine- | STK19 | G11; RP1; | NM_032454 | ENSG00000166301 | 8859 |
| | protein kinase 19 (EC | | D6S60; | | | |
| | 2.7.1.37) (RP1 protein) | | D6S60E; | | | |
| | (G11 protein). | | HLA-RP1; | | | |
| | | | MGC117388 | | | |
| 714 | Transcription factor 19 | TCF19_HUMAN | SC1 | Q9Y242 | ENSG00000137310 | |
| | (Transcription factor | | | | | |
| | SC1). | | | | | |
| 715 | Pannexin-2 | PANX2 | hPANX2; | NM_052839 | ENSG00000073150 | 56666 |
| | | | MGC119432 | | | |
| 716 | | CTA- | bK243E7.C22.3 | | OTTHUMG00000030167 | |
| | | 243E7.3 | | | | |
| 717 | signal peptide-CUB | SCUBE1 | | NM_173050 | ENSG00000159307 | 80274 |
| | domian-EGF-related 1 | | | | | |
| 718 | Reticulon 4 receptor | RTN4R | NGR; | NM_023004 | ENSG00000040608 | 65078 |
| | precursor (Nogo | | NOGOR | | | |
| | receptor) (NgR) | | | | | |
| | (Nogo-66 receptor) | | | | | |
| 719 | Arylsulfatase A | ARSA | MLD | NM_000487 | ENSG00000100299 | 410 |
| | precursor (EC 3.1.6.8) | | | | | |
| | (ASA) (Cerebroside- | | | | | |
| | sulfatase) [Contains: | | | | | |
| | Arylsulfatase A | | | | | |
| | component B; | | | | | |
| | Arylsulfatase A | | | | | |
| | component C] | | | | | |
| 720 | glycoprotein Ib | GP1BB | CD42c | NM_000407 | OTTHUMG00000030191 | 2812 |
| | (platelet), beta | | | | | |
| | polypeptide | | | | | |
| 721 | | | | | | |
| 722 | | No gene | | | | |
| | | associated | | | | |
| 723 | Mitochondrial | SLC25A18 | GC2 | NM_031481 | ENSG00000182902 | 83733 |
| | glutamate carrier 2 | | | | | |
| | (Glutamate/H(+) | | | | | |
| | symporter 2) (Solute | | | | | |
| | carrier family 25 | | | | | |
| | member 18, | | | | | |
| | ENSG00000182902, | | | | | |
| | SLC25A18 | | | | | |
| 724 | Thioredoxin reductase | TXNRD2 | TR; TR3; | NM_006440.2 | ENSG00000184470 | 10587 |
| | 2, mitochondrial | | SELZ; | | | |
| | precursor (EC 1.8.1.9) | | TRXR2; | | | |
| | (TR3) (TR-beta) | | TR-BETA | | | |
| | (Selenoprotein Z) | | | | | |
| | (SelZ) | | | | | |
| 725 | Somatostatin receptor | SSTR3 | | NM_001051 | ENSG00000183473 | 6753 |
| | type 3 (SS3R) (SSR- | | | | | |
| | 28) | | | | | |
| 726 | | RP11- | bA191L9.C22.1 | | OTTHUMG00000030964 | |
| | | 191L9.1 | | | | |
| 727 | No description- | AP000357.3 | Em: AP000357.C22.3 | | OTTHUMG00000030574 | |
| | pseudogene | | | | | |
| 728 | Cat eye syndrome | CECR1 | IDGFL | NM_017424.2 | ENST00000262607 | 51816 |
| | critical region protein | | | | | |
| | 1 precursor | | | | | |
| 729 | | No gene | | | | |
| | | associated | | | | |
| 730 | Membrane protein | MLC1 | VL; LVM; | NM_015166 | ENSG00000100427 | 23209 |
| | MLC1 | | MLC; | | | |
| | | | KIAA0027 | | | |
| 731 | BAI1-associated | BAIAP2L2 | FLJ22582 | NM_025045.3 | ENSG00000128298 | 80115 |
| | protein 2-like 2 | | | | | |
| 732 | No description | NP_056185.1 | | | ENSG00000100249 | |
| 733 | No description | RP4- | dJ695O20B.C22.9 | | OTTHUMG00000030111 | |
| | | 695O20_B.9 | | | | |
| 734 | OTTHUMG00000030167, | CTA- | bK243E7.C22.3 | No | OTTHUMG00000030167 | No Available |
| | CTA-243E7.3 | 243E7.3 | | Available | | |
| | | (Vega gene | | | | |
| | | ID) | | | | |
| 735 | novel transcript | XXbac- | Em: AC006547.C22.7 | | OTTHUMG00000030620 | |
| | | B444P24.7 | | | | |
| 736 | | LL22NC03- | | | OTTHUMG00000030676 | |
| | | 121E8.1- | | | | |
| | | 001 | | | | |
| 737 | No description | Q6ZN90_HUMAN | | | ENSG00000197549 | |
| 738 | NFAT activation | NFAM1 | CNAIP; | NM_145912 | ENSG00000167087 | 150372 |
| | molecule 1 precursor | | FLJ40652 | | | |
| | (Calcineurin/NFAT- | | | | | |
| | activating ITAM- | | | | | |
| | containing protein) | | | | | |
| | (NFAT activating | | | | | |
| | protein with ITAM | | | | | |
| | motif 1). | | | | | |
| 739 | immunoglobulin | IGLC2 | IGLC; | | OTTHUMG00000030352 | 3538 |
| | lambda constant 2 | | MGC20392; | | | |
| | | | MGC45681; | | | |
| | | | Ig light- | | | |
| | | | chain, | | | |
| | | | partial Ke- | | | |
| | | | Oz- | | | |
| | | | polypeptide, | | | |
| | | | C-term; | | | |
| | | | immunoglobulin | | | |
| | | | lambda | | | |
| | | | constant | | | |
| | | | region 2 | | | |
| | | | (Kern-Oz- | | | |
| | | | marker) | | | |
| 740 | immunoglobulin | IGLC2 | IGLC; | | OTTHUMG00000030352 | 3538 |
| | lambda constant 2 | | MGC20392; | | | |
| | | | MGC45681; | | | |
| | | | Ig light- | | | |
| | | | chain, | | | |
| | | | partial Ke- | | | |
| | | | Oz- | | | |
| | | | polypeptide, | | | |
| | | | C-term; | | | |
| | | | immunoglobulin | | | |
| | | | lambda | | | |
| | | | constant | | | |
| | | | region 2 | | | |
| | | | (Kern-Oz- | | | |
| | | | marker) | | | |
| 741 | OTTHUMG00000030870, | CTA- | bK503F6.C22.1 | No | OTTHUMG00000030870 | No Available |
| | CTA-503F6.1 | 503F6.1 | | Available | | |
| | | (Vega_gene | | | | |
| | | ID) | | | | |
| 742 | Lactosylceramide 4- | A4GALT | P1; PK; | NM_017436 | ENSG00000128274 | 53947 |
| | alpha- | | A14GALT; | | | |
| | galactosyltransferase | | A4GALT1 | | | |
| | (EC 2.4.1.228) | | | | | |
| 743 | | RP11- | | | OTTHUMG00000030966 | |
| | | 191L9.3 | | | | |
| 744 | Cold shock domain | CSDC2_HUMAN | PIPPIN | Q9Y534 | ENSG00000172346 | |
| | protein C2 (RNA- | | | | | |
| | binding protein | | | | | |
| | PIPPin) | | | | | |
| 745 | | GAS2L1 | GAR22; | NM_152236.1 | ENSG00000185340 | 10634 |
| | | | MGC17243 | | | |
| 746 | BAI1-associated | BAIAP2L2 | FLJ22582 | NM_025045.3 | ENSG00000128298 | 80115 |
| | protein 2-like 2 | | | | | |
| 747 | | NP_997360.1 | | | ENSG00000197182 | |
| 748 | OTTHUMG00000030991, | LL22NC03- | dJ671O14.C22.6; | No | OTTHUMG00000030991 | No Available |
| | LL22NC03- | 75B3.6 | KIAA1644 | Available | | |
| | 75B3.6 | (Vega gene | | | | |
| | | ID) | | | | |
| 749 | Reticulon 4 receptor | RTN4R | NGR; | NM_023004 | ENSG00000040608 | 65078 |
| | precursor (Nogo | | NOGOR | | | |
| | receptor) (NgR) | | | | | |
| | (Nogo-66 receptor) | | | | | |
| 750 | Smoothelin | SMTN | | NM_134269 | ENSG00000183963 | 6525 |
| 751 | solute carrier family | SLC35E4 | | NM_001001479.1 | ENSG00000100036 | 339665 |
| | 35, member E4 | | | | | |
| 752 | protein C22orf13 | CV013_HUMAN | | Q96NT3 | ENSG00000138867 | |
| | (Protein LLN4) | | | | | |
| 753 | | No gene | | | | |
| | | associated | | | | |
| 754 | Histone | HIST1H3A | H3/A; | NM_003532.2 | ENSG00000196966 | 8350 |
| | | | H3FA | | | |
| 755 | Gamma-aminobutyric- | GABRR1 ( | | NM_002042 | ENSG00000146276 | 2569 |
| | acid receptor rho-1 | | | | | |
| | subunit precursor | | | | | |
| | (GABA(A) receptor). | | | | | |
| 756 | OTTHUMG00000015693, | RP11- | bA12A2.3 | No | OTTHUMG00000015693 | No Available |
| | RP11-12A2.3 | 12A2.3 | | Available | | |
| | | (Vega_gene | | | | |
| | | ID) | | | | |
| 757 | | RP5- | dJ899B16.1 | | OTTHUMG00000015697 | |
| | | 899B16.1 ( | | | | |
| 758 | No description | RP11- | bA146I2.2 | | OTTHUMG00000014289 | |
| | | 146I2.2 | | | | |
| 759 | | NP_060483.2 | | | ENSG00000178289 | |
| 760 | Forkhead box protein | FOXO3A | AF6q21; | NM_001455 | ENSG00000118689 | 2309 |
| | O3A, | | FKHRL1; | | | |
| | ENSG00000118689, | | FKHRL1P2; | | | |
| | FOXO3A | | MGC12739; | | | |
| | | | MGC31925; | | | |
| | | | DKFZp781A0677 | | | |
| 761 | nuclear receptor | NCOA7 | ESNA1; | XM_059748 | ENSG00000111912 | 135112 |
| | coactivator 7 | | ERAP140; | | | |
| | | | MGC88425; | | | |
| | | | Nbla00052; | | | |
| | | | Nbla10993; | | | |
| | | | dJ187J11.3 | | | |
| 762 | | RP11- | bA554D15.1 | | OTTHUMG00000015043 | |
| | | 554D15.1 | | | | |
| 763 | chromosome 6 open | C6orf190 | C6orf207; | XM_069189 | OTTHUMG00000015534 | 387357 |
| | reading frame 190 | | FLJ40584; | | | |
| | | | bA325O24.3; | | | |
| | | | bA325O24.4 | | | |
| 764 | phosphatase and actin | PHACTR2 | C6orf56; | NM_014721 | OTTHUMG00000015732 | 9749 |
| | regulator 2 | | KIAA0680; | | | |
| | | | DKFZp686F18175 | | | |
| 765 | High mobility group | HMGA1 | HMG-R; | NM_002131 | ENSG00000137309 | 3159 |
| | protein HMG-I/HMG- | | HMGIY; | | | |
| | Y (HMG-I(Y)) (High | | MGC4242; | | | |
| | mobility group AT- | | MGC4854; | | | |
| | hook 1) (High mobility | | MGC12816 | | | |
| | group protein A1), | | | | | |
| | ENSG00000137309, | | | | | |
| | HMGA1 | | | | | |
| 766 | Pantetheinase | VNN1 | Tiff66; | NM_004666 | ENSG00000112299 | 8876 |
| | precursor (EC 3.5.1.—), | | MGC116930; | | | |
| | ENSG00000112299, | | MGC116931; | | | |
| | VNN1 | | MGC116932; | | | |
| | | | MGC116933 | | | |
| 767 | histone H2A | HIST1H2AA | H2AFR; | NM_170745 | ENSG00000164508 | 221613 |
| | | | bA317E16.2 | | | |
| 768 | transcription factor | TFAP2A | AP-2; | NM_003220 | OTTHUMG00000014235 | 7020 |
| | AP-2 alpha (activating | | AP2TF; | | | |
| | enhancer binding | | TFAP2; | | | |
| | protein 2 alpha) | | AP-2alpha | | | |
| 769 | N-acetyllactosaminide | GCNT2 | II, | NM_001491 | ENSG00000111846 | 2651 |
| | beta-1,6-N- | | GCNT5; | | | |
| | acetylglucosaminyl- | | II; IGNT; | | | |
| | tansferase (EC | | ULG3; | | | |
| | 2.4.1.150), | | AIGnT; | | | |
| | ENSG00000111846, | | BIGnT; | | | |
| | GCNT2 | | CIGnT; | | | |
| | | | GCNT5; | | | |
| | | | NAGCT1; | | | |
| | | | bA421M1.1; | | | |
| | | | bA360O19.2 | | | |
| 770 | | No gene | | | | |
| | | associated | | | | |
| 771 | | No gene | | | | |
| | | associated | | | | |
| 772 | | No gene | | | | |
| | | associated | | | | |
| 773 | | No gene | | | | |
| | | associated | | | | |
| 774 | | No gene | | | | |
| | | associated | | | | |
| 775 | | No gene | | | | |
| | | associated | | | | |
| 776 | | No gene | | | | |
| | | associated | | | | |
| 777 | | No gene | | | | |
| | | associated | | | | |
| 778 | | No gene | | | | |
| | | associated | | | | |
| 779 | | No gene | | | | |
| | | associated | | | | |
| 780 | No description | RP11- | bA318C17.1 | | OTTHUMG00000031920 | |
| | | 318C17.1 | | | | |
| 781 | | No gene | | | | |
| | | associated | | | | |
| 782 | | No gene | | | | |
| | | associated | | | | |
| 783 | | No gene | | | | |
| | | associated | | | | |
| 784 | | No gene | | | | |
| | | associated | | | | |
| 785 | | No gene | | | | |
| | | associated | | | | |
| 786 | | No gene | | | | |
| | | associated | | | | |
| 787 | novel transcript | RP11- | | | OTTHUMG00000032045 | |
| | | 216C10.1 | | | | |
| 788 | | No gene | | | | |
| | | associated | | | | |
| 789 | | No gene | | | | |
| | | associated | | | | |
| 790 | | No gene | | | | |
| | | associated | | | | |
| 791 | | RP11- | bA410N8.3 | | OTTHUMG00000032221 | |
| | | 410N8.3 | | | | |
| 792 | | TIMP3 ( | SFD; | NM_000362 | ENSG00000100234 | 7078 |
| | | | K222; | | | |
| | | | K222TA2; | | | |
| | | | HSMRK222 | | | |
| 793 | | No gene | | | | |
| | | associated | | | | |
| 794 | | No gene | | | | |
| | | associated | | | | |
| 795 | | No gene | | | | |
| | | associated | | | | |
| 796 | | No gene | | | | |
| 797 | no gene associated | No gene | | | | |
| | | associated | | | | |
| 798 | | No gene | | | | |
| | | associated | | | | |
| 799 | no gene | No gene | | | | |
| | | associated | | | | |
| 800 | | No gene | | | | |
| | | associated | | | | |
| 801 | | No gene | | | | |
| | | associated | | | | |
| 802 | | No gene | | | | |
| | | associated | | | | |
| 803 | | No gene | | | | |
| | | associated | | | | |
| 804 | sorting nexin 5 | SNX5 | FLJ10931 | NM_014426 | OTTHUMG00000031953 | 27131 |
| 805 | Probable D-tyrosyl- | HARS2 | DUEB; | NM_080820 | ENSG00000125821 | 92675 |
| | tRNA(Tyr) deacylase | | C20orf88; | | | |
| | (EC 3.1.—.—) | | MGC41905; | | | |
| | | | MGC119131; | | | |
| | | | bA379J5.3; | | | |
| | | | bA555E18.1 | | | |
| 806 | solute carrier family | SLC24A3 | NCKX3 | NM_020689 | OTTHUMG00000031993 | 57419 |
| | 24 | | | | | |
| | (sodiumVpotassiumVcalcium | | | | | |
| | exchanger), | | | | | |
| | member 3, | | | | | |
| | OTTHUMG00000031993, | | | | | |
| | SLC24A3 | | | | | |
| 807 | | CT026_HUMAN | C20orf26, | NM_015585.2 | ENSG00000089101 | 26074 |
| | | | dJ1178H5.4; | | | |
| | | | DKFZP434K156 | | | |
| 808 | RNA-binding protein | RALY | P542; | NM_007367 | ENSG00000125970 | 22913 |
| | Raly (hnRNP | | MGC117312 | | | |
| | associated with lethal | | | | | |
| | yellow homolog), D | | | | | |
| | ENSG00000125970, | | | | | |
| | RALY | | | | | |
| 809 | Protein phosphatase 1 | PPP1R16B | TIMAP; | NM_015568 | ENSG00000101445 | 26051 |
| | regulatory inhibitor | | ANKRD4; | | | |
| | subunit 16B (TGF- | | KIAA0823 | | | |
| | beta-inhibited | | | | | |
| | membrane-associated | | | | | |
| | protein) (hTIMAP) | | | | | |
| | (CAAX box protein | | | | | |
| | TIMAP) (Ankyrin | | | | | |
| | repeat domain protein | | | | | |
| | 4) | | | | | |
| 810 | protein tyrosine | PTPRT | RPTPrho; | NM_007050 | OTTHUMG00000033040 | 11122 |
| | phosphatase, receptor | | KIAA0283 | | | |
| | type, T | | | | | |
| 811 | protein tyrosine | PTPRT | RPTPrho; | NM_007050 | OTTHUMG00000033040 | 11122 |
| | phosphatase, receptor | | KIAA0283 | | | |
| | type, T | | | | | |
| 812 | protein tyrosine | PTPRT | RPTPrho; | NM_007050 | OTTHUMG00000033040 | 11122 |
| | phosphatase, receptor | | KIAA0283 | | | |
| | type, T | | | | | |
| 813 | Receptor-type | PTPRT | RPTPrho; | NM_007050 | ENSG00000196090 | 11122 |
| | tyrosine-protein | | KIAA0283 | | | |
| | phosphatase T | | | | | |
| | precursor (EC | | | | | |
| | 3.1.3.48) (R-PTP-T) | | | | | |
| | (RPTP-rho) | | | | | |
| 814 | cadherin-like 22 | CDH22 | C20orf25; | NM_021248 | OTTHUMG00000033073 | 64405 |
| | | | MGC39564; | | | |
| | | | dJ998H6.1 | | | |
| 815 | potassium voltage- | KCNB1 | DRK1; | NM_004975 | OTTHUMG00000033051 | 3745 |
| | gated channel, Shab- | | KV2.1; h- | | | |
| | related subfamily, | | DRK1 | | | |
| | member 1 | | | | | |
| 816 | potassium voltage- | KCNB1 | DRK1; | NM_004975 | OTTHUMG00000033051 | 3745 |
| | gated channel, Shab- | | KV2.1; h- | | | |
| | related subfamily, | | DRK1 | | | |
| | member 1 | | | | | |
| 817 | Zinc finger protein | SNAI1 | SNA; | NM_005985 | ENSG00000124216 | 6615 |
| | SNAI1 (Snail protein | | SNAH; | | | |
| | homolog) (Sna | | SLUGH2; | | | |
| | protein) | | dJ710H13.1 | | | |
| 818 | Cadherin-4 precursor | CDH4 | CAD4; | NM_001794 | ENSG00000179242 | 1002 |
| | (Retinal-cadherin) (R- | | RCAD; | | | |
| | cadherin) (R-CAD) | | FLJ22202; | | | |
| | | | FLJ40547; | | | |
| | | | MGC126700 | | | |
| 819 | cadherin 4, type 1, R- | CDH4 | CAD4; | NM_001794 | OTTHUMG00000032890 | 1002 |
| | cadherin (retinal) | | RCAD; | | | |
| | | | FLJ22202; | | | |
| | | | FLJ40547; | | | |
| | | | MGC126700 | | | |
| 820 | Cadherin-4 precursor | CDH4 | CAD4; | NM_001794 | ENSG00000179242 | 1002 |
| | (Retinal-cadherin) (R- | | RCAD; | | | |
| | cadherin) (R-CAD) | | FLJ22202; | | | |
| | | | FLJ40547; | | | |
| | | | MGC126700 | | | |
| 821 | Metalloproteinase | | | | | |
| | inhibitor 3 precursor | | | | | |
| | (TIMP-3) (Tissue | | | | | |
| | inhibitor of | | | | | |
| | metalloproteinases-3) | | | | | |
| | (MIG-5 protein). | | | | | |
| 822 | Tubulin alpha-8 chain | TUBA8 | TUBAL2 | NM_018943 | ENSG00000070490 | 51807 |
| | (Alpha-tubulin 8) | | | | | |
| 823 | | No gene | | | | |
| | | associated | | | | |
| 824 | | No gene | | | | |
| | | associated | | | | |

TABLE 6
Overview of the genomic regions and ranges of methylation within which the actual value of
methylation of said genomic regions lies specific of the said cells, tissues and/or organs.
The actual value of methylation is shown in FIGS. 1.1-1.403.

| SEQ | | | | | | |
|---|---|---|---|---|---|---|
| ID | CD4 | CD8 | | Embryonic | | |
| NO: | T- | T- | Embryonic | Skeletal | | Heart |
| Genomic | lymphocyte | lymphocyte | Liver | Muscle | Fibroblast | Muscle |
| 413 | 75-100% | 75-100% | 75-100% | 75-100% | 75-100% | 75-100% |
| 414 | 75-100% | 75-100% | 75-100% | 75-100% | 75-100% | 75-100% |
| 415 | 75-100% | 75-100% | 75-100% | 75-100% | 75-100% | 75-100% |
| 416 | 75-100% | 75-100% | 0-25% | 0-25% | 0-25% | 0-25% |
| 417 | 0-25% | 0-25% | 75-100% | 75-100% | 75-100% | 75-100% |
| 418 | 75-100% | 75-100% | 75-100% | 75-100% | 75-100% | 75-100% |
| 419 | 75-100% | 75-100% | 75-100% | 75-100% | 75-100% | 75-100% |
| 420 | 75-100% | 75-100% | 75-100% | 0-25% | 75-100% | 75-100% |
| 421 | 75-100% | 75-100% | 75-100% | 75-100% | 75-100% | 75-100% |
| 422 | 25-75% | 25-75% | 25-75% | 25-75% | 0-25% | 25-75% |
| 423 | 0-25% | 0-25% | 0-25% | 0-25% | 0-25% | 0-25% |
| 424 | 75-100% | 75-100% | 75-100% | 25-75% | 75-100% | 25-75% |
| 425 | 75-100% | 75-100% | 25-75% | 25-75% | 25-75% | 25-75% |
| 426 | 25-75% | 25-75% | 0-25% | 0-25% | 0-25% | 0-25% |
| 427 | 75-100% | 75-100% | 75-100% | 75-100% | 75-100% | 75-100% |
| 428 | 75-100% | 75-100% | 75-100% | 75-100% | 75-100% | 75-100% |
| 429 | 0-25% | 0-25% | 0-25% | 75-100% | 75-100% | 25-75% |
| 430 | 75-100% | 75-100% | 75-100% | 75-100% | 75-100% | 25-75% |
| 431 | 75-100% | 75-100% | 75-100% | 75-100% | 75-100% | 75-100% |
| 432 | 75-100% | 75-100% | 75-100% | 75-100% | 75-100% | 75-100% |
| 433 | 75-100% | 75-100% | 75-100% | 75-100% | 75-100% | 75-100% |
| 434 | 75-100% | 75-100% | 75-100% | 75-100% | 75-100% | 75-100% |
| 435 | 75-100% | 75-100% | 75-100% | 75-100% | 75-100% | 75-100% |
| 436 | 75-100% | 75-100% | 75-100% | 75-100% | 75-100% | 75-100% |
| 437 | 75-100% | 75-100% | 75-100% | 75-100% | 75-100% | 75-100% |
| 438 | 75-100% | 75-100% | 25-75% | 25-75% | 25-75% | 25-75% |
| 439 | 25-75% | 25-75% | 25-75% | 25-75% | 25-75% | 25-75% |
| 440 | 75-100% | 75-100% | 75-100% | 75-100% | 0-25% | 75-100% |
| 441 | 75-100% | 75-100% | 75-100% | 25-75% | 75-100% | 75-100% |
| 442 | 75-100% | 75-100% | 25-75% | 25-75% | 25-75% | 25-75% |
| 443 | 75-100% | 75-100% | 75-100% | 75-100% | 75-100% | 75-100% |
| 444 | 75-100% | 75-100% | 75-100% | 25-75% | 75-100% | 75-100% |
| 445 | 75-100% | 75-100% | 0-25% | 0-25% | 0-25% | 0-25% |
| 446 | 75-100% | 75-100% | 75-100% | 75-100% | 0-25% | 75-100% |
| 447 | 75-100% | 75-100% | 75-100% | 75-100% | 75-100% | 75-100% |
| 448 | 25-75% | 25-75% | 25-75% | 25-75% | 25-75% | 25-75% |
| 449 | 0-25% | 0-25% | 75-100% | 75-100% | 75-100% | 75-100% |
| 450 | 25-75% | 25-75% | 25-75% | 25-75% | 75-100% | 75-100% |
| 451 | 0-25% | 0-25% | 75-100% | 75-100% | 75-100% | 75-100% |
| 452 | 0-25% | 0-25% | 75-100% | 75-100% | 75-100% | 75-100% |
| 453 | 75-100% | 75-100% | 75-100% | 75-100% | 75-100% | 75-100% |
| 454 | 75-100% | 75-100% | 25-75% | 25-75% | 25-75% | 25-75% |
| 455 | 75-100% | 75-100% | 75-100% | 75-100% | 75-100% | 75-100% |
| 456 | 75-100% | 75-100% | 75-100% | 75-100% | 75-100% | 75-100% |
| 457 | 75-100% | 75-100% | 75-100% | 25-75% | 75-100% | 75-100% |
| 458 | 0-25% | 0-25% | 75-100% | 75-100% | 75-100% | 75-100% |
| 459 | 75-100% | 75-100% | 0-25% | 75-100% | 75-100% | 75-100% |
| 460 | 75-100% | 75-100% | 75-100% | 75-100% | 75-100% | 75-100% |
| 461 | 75-100% | 75-100% | 75-100% | 75-100% | 75-100% | 75-100% |
| 462 | 75-100% | 75-100% | 75-100% | 75-100% | 25-75% | 75-100% |
| 463 | 0-25% | 0-25% | 25-75% | 25-75% | 25-75% | 25-75% |
| 464 | 75-100% | 75-100% | 75-100% | 75-100% | 75-100% | 75-100% |
| 465 | 75-100% | 75-100% | 75-100% | 0-25% | 0-25% | 0-25% |
| 466 | 75-100% | 75-100% | 75-100% | 75-100% | 75-100% | 75-100% |
| 467 | 75-100% | 75-100% | 25-75% | 25-75% | 25-75% | 25-75% |
| 468 | 75-100% | 75-100% | 25-75% | 25-75% | 0-25% | 25-75% |
| 469 | 75-100% | 75-100% | 75-100% | 75-100% | 75-100% | 75-100% |
| 470 | 0-25% | 0-25% | 0-25% | 0-25% | 0-25% | 0-25% |
| 471 | 75-100% | 75-100% | 75-100% | 75-100% | 25-75% | 25-75% |
| 472 | 75-100% | 75-100% | 75-100% | 75-100% | 75-100% | 75-100% |
| 473 | 75-100% | 75-100% | 75-100% | 75-100% | 25-75% | 75-100% |
| 474 | 0-25% | 0-25% | 75-100% | 75-100% | 75-100% | 75-100% |
| 475 | 75-100% | 75-100% | 25-75% | 75-100% | 75-100% | 75-100% |
| 476 | 75-100% | 75-100% | 25-75% | 25-75% | 75-100% | 75-100% |
| 477 | 75-100% | 75-100% | 25-75% | 0-25% | 0-25% | 25-75% |
| 478 | 75-100% | 75-100% | 25-75% | 25-75% | 25-75% | 25-75% |
| 479 | 75-100% | 75-100% | 25-75% | 0-25% | 0-25% | 25-75% |
| 480 | 25-75% | 25-75% | 25-75% | 75-100% | 75-100% | 75-100% |
| 481 | 75-100% | 75-100% | 25-75% | 25-75% | 25-75% | 25-75% |
| 482 | 75-100% | 75-100% | 25-75% | 25-75% | 25-75% | 25-75% |
| 483 | 75-100% | 75-100% | 25-75% | 25-75% | 25-75% | 25-75% |
| 484 | 75-100% | 75-100% | 25-75% | 0-25% | 0-25% | 25-75% |
| 485 | 75-100% | 75-100% | 75-100% | 0-25% | 0-25% | 0-25% |
| 486 | 0-25% | 0-25% | 0-25% | 0-25% | 0-25% | 0-25% |
| 487 | 75-100% | 75-100% | 75-100% | 25-75% | 25-75% | 75-100% |
| 488 | 75-100% | 75-100% | 75-100% | 25-75% | 75-100% | 75-100% |
| 489 | 0-25% | 0-25% | 25-75% | 25-75% | 25-75% | 25-75% |
| 490 | 0-25% | 0-25% | 75-100% | 75-100% | 75-100% | 75-100% |
| 491 | 75-100% | 75-100% | 0-25% | 0-25% | 0-25% | 0-25% |
| 492 | 75-100% | 75-100% | 0-25% | 0-25% | 0-25% | 0-25% |
| 493 | 0-25% | 0-25% | 25-75% | 25-75% | 75-100% | 75-100% |
| 494 | 75-100% | 75-100% | 75-100% | 75-100% | 75-100% | 75-100% |
| 495 | 75-100% | 75-100% | 75-100% | 75-100% | 75-100% | 75-100% |
| 496 | 75-100% | 75-100% | 25-75% | 25-75% | 25-75% | 25-75% |
| 497 | 0-25% | 0-25% | 75-100% | 75-100% | 75-100% | 75-100% |
| 498 | 75-100% | 75-100% | 75-100% | 75-100% | 75-100% | 75-100% |
| 499 | 75-100% | 75-100% | 75-100% | 75-100% | 75-100% | 75-100% |
| 500 | 75-100% | 75-100% | 75-100% | 75-100% | 75-100% | 75-100% |
| 501 | 25-75% | 25-75% | 25-75% | 25-75% | 25-75% | 25-75% |
| 502 | 25-75% | 25-75% | 25-75% | 25-75% | 25-75% | 25-75% |
| 503 | 75-100% | 75-100% | 75-100% | 75-100% | 75-100% | 75-100% |
| 504 | 75-100% | 75-100% | 75-100% | 75-100% | 0-25% | 75-100% |
| 505 | 0-25% | 0-25% | 75-100% | 0-25% | 0-25% | 0-25% |
| 506 | 75-100% | 75-100% | 75-100% | 75-100% | 75-100% | 75-100% |
| 507 | 75-100% | 75-100% | 75-100% | 75-100% | 75-100% | 75-100% |
| 508 | 0-25% | 0-25% | 0-25% | 0-25% | 0-25% | 0-25% |
| 509 | 75-100% | 75-100% | 25-75% | 75-100% | 75-100% | 75-100% |
| 510 | 75-100% | 75-100% | 75-100% | 75-100% | 75-100% | 75-100% |
| 511 | 25-75% | 75-100% | 75-100% | 75-100% | 75-100% | 25-75% |
| 512 | 75-100% | 75-100% | 75-100% | 75-100% | 75-100% | 75-100% |
| 513 | 25-75% | 25-75% | 75-100% | 75-100% | 75-100% | 75-100% |
| 514 | 75-100% | 75-100% | 75-100% | 75-100% | 75-100% | 75-100% |
| 515 | 0-25% | 0-25% | 75-100% | 75-100% | 75-100% | 75-100% |
| 516 | 75-100% | 75-100% | 75-100% | 75-100% | 75-100% | 75-100% |
| 517 | 75-100% | 75-100% | 75-100% | 75-100% | 75-100% | 75-100% |
| 518 | 75-100% | 75-100% | 25-75% | 25-75% | 25-75% | 25-75% |
| 519 | 0-25% | 0-25% | 25-75% | 25-75% | 25-75% | 25-75% |
| 520 | 75-100% | 75-100% | 75-100% | 75-100% | 75-100% | 75-100% |
| 521 | 75-100% | 75-100% | 75-100% | 75-100% | 75-100% | 75-100% |
| 522 | 0-25% | 0-25% | 75-100% | 75-100% | 75-100% | 75-100% |
| 523 | 75-100% | 75-100% | 75-100% | 75-100% | 75-100% | 75-100% |
| 524 | 75-100% | 75-100% | 75-100% | 75-100% | 0-25% | 75-100% |
| 525 | 0-25% | 0-25% | ND | 0-25% | 75-100% | 0-25% |
| 526 | 75-100% | 75-100% | 75-100% | 75-100% | 75-100% | 75-100% |
| 527 | 75-100% | 75-100% | 0-25% | 0-25% | 0-25% | 0-25% |
| 528 | 0-25% | 0-25% | 75-100% | 75-100% | 75-100% | 75-100% |
| 529 | 75-100% | 75-100% | 75-100% | 75-100% | 75-100% | 75-100% |
| 530 | 75-100% | 75-100% | 75-100% | 75-100% | 75-100% | 75-100% |
| 531 | 0-25% | 0-25% | 25-75% | 25-75% | 25-75% | 25-75% |
| 532 | 75-100% | 75-100% | 75-100% | 75-100% | 75-100% | 75-100% |
| 533 | 75-100% | 75-100% | 75-100% | 75-100% | 75-100% | 75-100% |
| 534 | 75-100% | 75-100% | 75-100% | 75-100% | 25-75% | 75-100% |
| 535 | 0-25% | 0-25% | 75-100% | 75-100% | 75-100% | 75-100% |
| 536 | 75-100% | 75-100% | 75-100% | 75-100% | 75-100% | 75-100% |
| 537 | 25-75% | 25-75% | 0-25% | 0-25% | 0-25% | 25-75% |
| 538 | 0-25% | 0-25% | 0-25% | 0-25% | 0-25% | 0-25% |
| 539 | 75-100% | 75-100% | 75-100% | 75-100% | 75-100% | 75-100% |
| 540 | 75-100% | 75-100% | 75-100% | 75-100% | 75-100% | 75-100% |
| 541 | 25-75% | 25-75% | 25-75% | 25-75% | 25-75% | 25-75% |
| 542 | 75-100% | 75-100% | 25-75% | 25-75% | 25-75% | 25-75% |
| 543 | 75-100% | 75-100% | 75-100% | 75-100% | 75-100% | 75-100% |
| 544 | 75-100% | 75-100% | 75-100% | 75-100% | 75-100% | 75-100% |
| 545 | 0-25% | 0-25% | 0-25% | 0-25% | 0-25% | 0-25% |
| 546 | 0-25% | 0-25% | 0-25% | 0-25% | 0-25% | 0-25% |
| 547 | 75-100% | 75-100% | 75-100% | 75-100% | 75-100% | 75-100% |
| 548 | 75-100% | 75-100% | 75-100% | 75-100% | 75-100% | 75-100% |
| 549 | 75-100% | 75-100% | 75-100% | 75-100% | 75-100% | 75-100% |
| 550 | 75-100% | 75-100% | 75-100% | 75-100% | 75-100% | 75-100% |
| 551 | 75-100% | 75-100% | 75-100% | 75-100% | 75-100% | 75-100% |
| 552 | 75-100% | 75-100% | 25-75% | 75-100% | 75-100% | 75-100% |
| 553 | 75-100% | 75-100% | 75-100% | 75-100% | 75-100% | 75-100% |
| 554 | 0-25% | 0-25% | 75-100% | 75-100% | 75-100% | 75-100% |
| 555 | 75-100% | 75-100% | 0-25% | 0-25% | 0-25% | 0-25% |
| 556 | 75-100% | 75-100% | 75-100% | 75-100% | 75-100% | 75-100% |
| 557 | 75-100% | 75-100% | 25-75% | 25-75% | 25-75% | 25-75% |
| 558 | 75-100% | 75-100% | 0-25% | 0-25% | 0-25% | 0-25% |
| 559 | 75-100% | 75-100% | 75-100% | 75-100% | 75-100% | 75-100% |
| 560 | 75-100% | 75-100% | 75-100% | 75-100% | 75-100% | 75-100% |
| 561 | 75-100% | 75-100% | 75-100% | 75-100% | 75-100% | 25-75% |
| 562 | 0-25% | 0-25% | 75-100% | 75-100% | 75-100% | 75-100% |
| 563 | 75-100% | 75-100% | 75-100% | 75-100% | 25-75% | 75-100% |
| 564 | 25-75% | 25-75% | 25-75% | 25-75% | 75-100% | 25-75% |
| 565 | 75-100% | 75-100% | 75-100% | 75-100% | 75-100% | 75-100% |
| 566 | 75-100% | 75-100% | ND | 25-75% | 0-25% | 25-75% |
| 567 | 75-100% | 75-100% | 75-100% | 75-100% | 25-75% | 75-100% |
| 568 | 0-25% | 0-25% | 0-25% | 0-25% | 0-25% | 0-25% |
| 569 | 75-100% | 75-100% | 75-100% | 75-100% | 0-25% | 25-75% |
| 570 | 75-100% | 75-100% | 75-100% | 75-100% | 75-100% | 75-100% |
| 571 | 75-100% | 75-100% | 25-75% | 25-75% | 25-75% | 25-75% |
| 572 | 25-75% | 25-75% | 25-75% | 25-75% | 75-100% | 25-75% |
| 573 | 75-100% | 75-100% | 75-100% | 75-100% | 75-100% | 75-100% |
| 574 | 0-25% | 0-25% | 75-100% | 75-100% | 75-100% | 75-100% |
| 575 | 75-100% | 75-100% | 75-100% | 75-100% | 75-100% | 75-100% |
| 576 | 0-25% | 0-25% | 0-25% | 0-25% | 75-100% | 0-25% |
| 577 | 75-100% | 75-100% | ND | 75-100% | 75-100% | 75-100% |
| 578 | 75-100% | 75-100% | 75-100% | 75-100% | 75-100% | 75-100% |
| 579 | 0-25% | 0-25% | 0-25% | 0-25% | 0-25% | 25-75% |
| 580 | 75-100% | 75-100% | 75-100% | 75-100% | 0-25% | 75-100% |
| 581 | 75-100% | 75-100% | 75-100% | 75-100% | 75-100% | 75-100% |
| 582 | 75-100% | 75-100% | 75-100% | 75-100% | 75-100% | 75-100% |
| 583 | 75-100% | 75-100% | 75-100% | 75-100% | 75-100% | 75-100% |
| 584 | 0-25% | 0-25% | 0-25% | 0-25% | 75-100% | 0-25% |
| 585 | 75-100% | 75-100% | 75-100% | 75-100% | 75-100% | 75-100% |
| 586 | 75-100% | 75-100% | 75-100% | 75-100% | 0-25% | 75-100% |
| 587 | 75-100% | 75-100% | 75-100% | 75-100% | 75-100% | 75-100% |
| 588 | 75-100% | 75-100% | 25-75% | 0-25% | 0-25% | 25-75% |
| 589 | 0-25% | 0-25% | 0-25% | 0-25% | 0-25% | 0-25% |
| 590 | 25-75% | 25-75% | 0-25% | 0-25% | 0-25% | 25-75% |
| 591 | 0-25% | 0-25% | 0-25% | 0-25% | 0-25% | 0-25% |
| 592 | 75-100% | 75-100% | 75-100% | 75-100% | 75-100% | 75-100% |
| 593 | 75-100% | 75-100% | 25-75% | 0-25% | 0-25% | 25-75% |
| 594 | 75-100% | 75-100% | 75-100% | 75-100% | 0-25% | 75-100% |
| 595 | 75-100% | 75-100% | 75-100% | 75-100% | 75-100% | 25-75% |
| 596 | 75-100% | 75-100% | 75-100% | 25-75% | 0-25% | 75-100% |
| 597 | 75-100% | 75-100% | 0-25% | 0-25% | 0-25% | 0-25% |
| 598 | 0-25% | 0-25% | 75-100% | 75-100% | 75-100% | 75-100% |
| 599 | 75-100% | 75-100% | 75-100% | 75-100% | 25-75% | 75-100% |
| 600 | 75-100% | 75-100% | 75-100% | 75-100% | 75-100% | 75-100% |
| 601 | 75-100% | 75-100% | 75-100% | 75-100% | 25-75% | 75-100% |
| 602 | 75-100% | 75-100% | 75-100% | 75-100% | 75-100% | 75-100% |
| 603 | 25-75% | 75-100% | 75-100% | 75-100% | 75-100% | 75-100% |
| 604 | 75-100% | 75-100% | 75-100% | 75-100% | 75-100% | 75-100% |
| 605 | 75-100% | 75-100% | 75-100% | 75-100% | 75-100% | 75-100% |
| 606 | 75-100% | 75-100% | 75-100% | 75-100% | 75-100% | 75-100% |
| 607 | 0-25% | 0-25% | 75-100% | 75-100% | 75-100% | 75-100% |
| 608 | 75-100% | 75-100% | 75-100% | 75-100% | 75-100% | 75-100% |
| 609 | 75-100% | ND | ND | ND | 0-25% | 75-100% |
| 610 | 75-100% | 75-100% | 75-100% | 75-100% | 0-25% | 75-100% |
| 611 | 75-100% | 75-100% | 75-100% | 75-100% | 0-25% | 75-100% |
| 612 | 75-100% | 75-100% | 75-100% | 25-75% | 75-100% | 75-100% |
| 613 | 0-25% | 0-25% | 0-25% | 0-25% | 0-25% | 0-25% |
| 614 | 25-75% | 25-75% | 25-75% | 25-75% | 25-75% | 25-75% |
| 615 | 75-100% | 75-100% | 75-100% | 75-100% | 75-100% | 25-75% |
| 616 | 25-75% | 25-75% | 25-75% | 25-75% | 25-75% | 25-75% |
| 617 | 75-100% | 75-100% | 75-100% | 75-100% | 0-25% | 75-100% |
| 618 | 75-100% | 75-100% | 75-100% | 75-100% | 75-100% | 75-100% |
| 619 | 0-25% | 0-25% | 0-25% | 0-25% | 0-25% | 75-100% |
| 620 | 75-100% | 75-100% | 75-100% | 75-100% | 75-100% | 75-100% |
| 621 | 75-100% | 75-100% | 75-100% | 25-75% | 75-100% | 75-100% |
| 622 | 75-100% | 75-100% | 75-100% | 25-75% | 25-75% | 25-75% |
| 623 | 0-25% | 0-25% | 0-25% | 0-25% | 0-25% | 0-25% |
| 624 | 75-100% | 75-100% | 75-100% | 25-75% | 75-100% | 75-100% |
| 625 | 75-100% | 75-100% | 75-100% | 75-100% | 75-100% | 75-100% |
| 626 | 75-100% | 75-100% | 25-75% | 25-75% | 0-25% | 75-100% |
| 627 | 75-100% | 75-100% | 75-100% | 75-100% | 75-100% | 75-100% |
| 628 | 75-100% | 75-100% | 75-100% | 75-100% | 75-100% | 75-100% |
| 629 | 25-75% | 25-75% | 25-75% | 25-75% | 25-75% | 25-75% |
| 630 | 75-100% | 75-100% | 75-100% | 75-100% | 25-75% | 75-100% |
| 631 | 75-100% | 75-100% | 75-100% | 75-100% | 0-25% | 75-100% |
| 632 | 75-100% | 75-100% | 25-75% | 25-75% | 25-75% | 25-75% |
| 633 | 0-25% | 0-25% | 0-25% | 0-25% | 0-25% | 0-25% |
| 634 | 75-100% | 25-75% | 25-75% | 25-75% | 25-75% | 25-75% |
| 635 | 75-100% | 75-100% | 75-100% | 75-100% | 75-100% | 75-100% |
| 636 | 75-100% | 75-100% | 75-100% | 75-100% | 25-75% | 75-100% |
| 637 | 25-75% | 0-25% | 0-25% | 0-25% | 0-25% | 0-25% |
| 638 | 75-100% | 75-100% | 75-100% | 75-100% | 25-75% | 75-100% |
| 639 | 25-75% | 25-75% | 75%-100% | 75-100% | 0-25% | 75-100% |
| 640 | 75-100% | 75-100% | 75-100% | 75-100% | 25-75% | 75-100% |
| 641 | 75-100% | 75-100% | 75-100% | 75-100% | 0-25% | 75-100% |
| 642 | 75-100% | 75-100% | 75-100% | 75-100% | 0-25% | 75-100% |
| 643 | 75-100% | 75-100% | 75-100% | 75-100% | 0-25% | 75-100% |
| 644 | 75-100% | 75-100% | 75-100% | 75-100% | 75-100% | 75-100% |
| 645 | 75-100% | 75-100% | 75-100% | 75-100% | 0-25% | 75-100% |
| 646 | 75-100% | 75-100% | 75-100% | 75-100% | 25-75% | 75-100% |
| 647 | 75-100% | 75-100% | 75-100% | 25-75% | 0-25% | 75-100% |
| 648 | 25-75% | 25-75% | 75-100% | 75-100% | 0-25% | 75-100% |
| 649 | 75-100% | 75-100% | 75-100% | 75-100% | 75-100% | 75-100% |
| 650 | 75-100% | 75-100% | 75-100% | 25-75% | 25-75% | 25-75% |
| 651 | 75-100% | 75-100% | 25-75% | 25-75% | 25-75% | 25-75% |
| 652 | 25-75% | 75-100% | 75-100% | 75-100% | 75-100% | 75-100% |
| 653 | 75-100% | 75-100% | 75-100% | 75-100% | 0-25% | 75-100% |
| 654 | 75-100% | 75-100% | 75-100% | 25-75% | 75-100% | 75-100% |
| 655 | 75-100% | 75-100% | 75-100% | 75-100% | 0-25% | 75-100% |
| 656 | 75-100% | 75-100% | 75-100% | 75-100% | 75-100% | 75-100% |
| 657 | 75-100% | 75-100% | 75-100% | 75-100% | 75-100% | 75-100% |
| 658 | 75-100% | 75-100% | 75-100% | 25-75% | 75-100% | 75-100% |
| 659 | 75-100% | 75-100% | 25-75% | 25-75% | 25-75% | 25-75% |
| 660 | 75-100% | 75-100% | 75-100% | 75-100% | 25-75% | 75-100% |
| 661 | 75-100% | 75-100% | 75-100% | 75-100% | 75-100% | 75-100% |
| 662 | 75-100% | 75-100% | 75-100% | 75-100% | 75-100% | 75-100% |
| 663 | 75-100% | 75-100% | 75-100% | 75-100% | 75-100% | 75-100% |
| 664 | 75-100% | 75-100% | 75-100% | 75-100% | 75-100% | 75-100% |
| 665 | 75-100% | 75-100% | 75-100% | ND | 25-75% | 25-75% |
| 666 | 75-100% | 75-100% | 75-100% | 75-100% | 75-100% | 75-100% |
| 667 | 75-100% | 75-100% | 75-100% | 75-100% | 75-100% | 75-100% |
| 668 | 75-100% | 75-100% | 75-100% | 75-100% | 75-100% | 75-100% |
| 669 | 75-100% | 75-100% | 25-75% | 25-75% | 0-25% | 0-25% |
| 670 | 75-100% | 75-100% | 75-100% | 75-100% | 75-100% | 75-100% |
| 671 | 75-100% | 75-100% | 75-100% | 75-100% | 75-100% | 75-100% |
| 672 | 75-100% | 75-100% | 75-100% | 75-100% | 75-100% | 75-100% |
| 673 | 75-100% | 75-100% | 25-75% | 25-75% | 25-75% | 25-75% |
| 674 | 75-100% | 75-100% | 75-100% | 75-100% | 75-100% | 75-100% |
| 675 | 75-100% | 75-100% | 25-75% | 25-75% | 25-75% | 25-75% |
| 676 | 75-100% | 75-100% | 0-25% | 0-25% | 0-25% | 0-25% |
| 677 | 0-25% | 0-25% | 0-25% | 0-25% | 0-25% | 0-25% |
| 678 | 75-100% | 75-100% | 75-100% | 25-75% | 75-100% | 75-100% |
| 679 | 0-25% | 0-25% | 0-25% | 75-100% | 75-100% | 75-100% |
| 680 | 25-75% | 25-75% | 25-75% | 25-75% | 25-75% | 25-75% |
| 681 | 0-25% | 0-25% | 0-25% | 0-25% | 0-25% | 0-25% |
| 682 | 75-100% | 75-100% | 0-25% | 0-25% | 0-25% | 25-75% |
| 683 | 0-25% | 0-25% | 75-100% | 75-100% | 75-100% | 75-100% |
| 684 | 0-25% | 0-25% | 0-25% | 0-25% | 0-25% | 0-25% |
| 685 | 75-100% | 75-100% | 25-75% | 25-75% | 25-75% | 25-75% |
| 686 | 75-100% | 75-100% | 75-100% | 75-100% | 75-100% | 75-100% |
| 687 | 25-75% | 25-75% | 25-75% | 25-75% | 25-75% | 25-75% |
| 688 | 25-75% | 25-75% | 25-75% | 25-75% | 0-25% | 25-75% |
| 689 | 75-100% | 75-100% | 75-100% | 75-100% | 75-100% | 75-100% |
| 690 | 75-100% | 75-100% | 75-100% | 25-75% | 0-25% | 75-100% |
| 691 | 75-100% | 75-100% | 0-25% | 0-25% | 0-25% | 0-25% |
| 692 | 75-100% | 75-100% | 75-100% | 75-100% | 75-100% | 75-100% |
| 693 | 75-100% | 75-100% | 75-100% | 75-100% | 75-100% | 75-100% |
| 694 | 75-100% | 75-100% | 75-100% | 75-100% | 75-100% | 75-100% |
| 695 | 0-25% | 0-25% | 25-75% | 75-100% | 75-100% | 75-100% |
| 696 | 25-75% | 25-75% | 25-75% | 25-75% | 25-75% | 0-25% |
| 697 | 0-25% | 0-25% | 75-100% | 75-100% | 75-100% | 75-100% |
| 698 | 75-100% | 75-100% | 75-100% | 75-100% | 75-100% | 25-75% |
| 699 | 75-100% | 75-100% | ND | ND | 25-75% | 75-100% |
| 700 | 0-25% | 0-25% | 0-25% | 0-25% | 0-25% | 75-100% |
| 701 | 0-25% | 0-25% | 75-100% | 75-100% | 75-100% | 75-100% |
| 702 | 75-100% | 75-100% | 75-100% | 75-100% | 75-100% | 75-100% |
| 703 | 25-75% | 25-75% | 75-100% | 75-100% | 75-100% | 75-100% |
| 704 | 75-100% | 75-100% | 25-75% | 25-75% | 25-75% | 25-75% |
| 705 | 75-100% | 75-100% | 75-100% | 75-100% | 75-100% | 75-100% |
| 706 | 25-75% | 75-100% | 25-75% | 25-75% | 25-75% | 25-75% |
| 707 | 75-100% | 75-100% | 75-100% | 75-100% | 75-100% | 75-100% |
| 708 | 75-100% | 75-100% | 75-100% | 75-100% | 0-25% | 75-100% |
| 709 | 75-100% | 75-100% | 75-100% | 75-100% | 0-25% | 75-100% |
| 710 | 0-25% | 0-25% | 25-75% | 25-75% | 75-100% | 75-100% |
| 711 | 75-100% | 75-100% | 75-100% | 75-100% | 75-100% | 75-100% |
| 712 | 75-100% | 25-75% | 25-75% | 75-100% | 75-100% | 75-100% |
| SEQ | | | | | | |
| ID | | | | | | |
| NO: | | | | | Skeletal | |
| Genomic | Keratinocyte | Liver | Melanocyte | Placenta | Muscle | Sperm |
| 413 | 75-100% | 75-100% | 75-100% | 75-100% | 75-100% | 0-25% |
| 414 | 75-100% | 75-100% | 75-100% | 75-100% | 75-100% | 0-25% |
| 415 | 75-100% | 25-75% | 75-100% | 75-100% | 75-100% | 75-100% |
| 416 | 0-25% | 0-25% | 0-25% | 0-25% | 0-25% | 0-25% |
| 417 | 75-100% | 75-100% | 75-100% | 75-100% | 75-100% | 75-100% |
| 418 | 75-100% | 75-100% | 0-25% | 75-100% | 75-100% | 75-100% |
| 419 | 75-100% | 75-100% | 75-100% | 0-25% | 75-100% | 0-25% |
| 420 | 75-100% | 75-100% | 75-100% | 75-100% | 0-25% | 75-100% |
| 421 | 75-100% | 75-100% | 75-100% | 75-100% | 75-100% | 0-25% |
| 422 | 75-100% | 25-75% | 75-100% | 25-75% | 25-75% | 0-25% |
| 423 | 0-25% | 0-25% | 75-100% | 0-25% | 0-25% | 75-100% |
| 424 | 75-100% | 75-100% | 75-100% | 25-75% | 25-75% | 75-100% |
| 425 | 25-75% | 75-100% | 75-100% | 25-75% | 25-75% | 75-100% |
| 426 | 0-25% | 0-25% | 0-25% | 0-25% | 0-25% | 0-25% |
| 427 | 75-100% | 25-75% | 75-100% | 75-100% | 75-100% | 0-25% |
| 428 | 75-100% | 75-100% | 75-100% | 75-100% | 75-100% | 0-25% |
| 429 | 25-75% | 0-25% | 0-25% | 75-100% | 75-100% | 0-25% |
| 430 | 75-100% | 75-100% | 75-100% | 75-100% | 75-100% | 75-100% |
| 431 | 75-100% | 75-100% | 75-100% | 75-100% | 75-100% | 0-25% |
| 432 | 75-100% | 75-100% | 75-100% | 75-100% | 25-75% | 75-100% |
| 433 | 25-75% | 75-100% | 75-100% | 75-100% | 75-100% | 75-100% |
| 434 | 75-100% | 25-75% | 75-100% | 75-100% | 75-100% | 0-25% |
| 435 | 25-75% | 75-100% | 75-100% | 75-100% | 75-100% | 0-25% |
| 436 | 75-100% | 75-100% | 75-100% | 25-75% | 75-100% | 75-100% |
| 437 | 75-100% | 75-100% | 75-100% | 75-100% | 75-100% | 0-25% |
| 438 | 25-75% | 25-75% | 25-75% | 25-75% | 25-75% | 0-25% |
| 439 | 75-100% | 25-75% | 25-75% | 25-75% | 25-75% | 25-75% |
| 440 | 75-100% | 75-100% | 75-100% | 75-100% | 25-75% | 75-100% |
| 441 | 75-100% | 75-100% | ND | 75-100% | 25-75% | 75-100% |
| 442 | 25-75% | 25-75% | 25-75% | 25-75% | 25-75% | 75-100% |
| 443 | 75-100% | 75-100% | 0-25% | 75-100% | 75-100% | 75-100% |
| 444 | 75-100% | 75-100% | 75-100% | 75-100% | 75-100% | 75-100% |
| 445 | 0-25% | 0-25% | 0-25% | 0-25% | 0-25% | 0-25% |
| 446 | 75-100% | 75-100% | 75-100% | 75-100% | 75-100% | 75-100% |
| 447 | 0-25% | 75-100% | 75-100% | 75-100% | 75-100% | 75-100% |
| 448 | 25-75% | 75-100% | 25-75% | 25-75% | 25-75% | 75-100% |
| 449 | 75-100% | 75-100% | 75-100% | 75-100% | 75-100% | 75-100% |
| 450 | 0-25% | 25-75% | 75-100% | 25-75% | 25-75% | ND |
| 451 | 75-100% | 75-100% | 75-100% | 75-100% | 75-100% | 75-100% |
| 452 | 75-100% | 75-100% | 75-100% | 75-100% | 75-100% | 75-100% |
| 453 | 75-100% | 75-100% | 75-100% | 75-100% | 0-25% | 0-25% |
| 454 | 0-25% | 75-100% | 75-100% | 75-100% | 25-75% | 75-100% |
| 455 | 75-100% | 75-100% | 0-25% | 75-100% | 75-100% | 75-100% |
| 456 | 75-100% | 25-75% | 75-100% | 75-100% | 75-100% | 75-100% |
| 457 | 75-100% | 25-75% | 75-100% | 75-100% | 75-100% | 75-100% |
| 458 | 75-100% | 75-100% | 75-100% | 75-100% | 75-100% | 75-100% |
| 459 | 75-100% | 75-100% | 75-100% | 75-100% | 75-100% | 75-100% |
| 460 | 0-25% | 75-100% | 75-100% | 75-100% | 75-100% | 75-100% |
| 461 | 0-25% | 75-100% | 75-100% | 75-100% | 75-100% | 75-100% |
| 462 | 0-25% | 75-100% | 75-100% | 75-100% | 25-75% | 75-100% |
| 463 | 0-25% | 25-75% | 25-75% | 25-75% | 25-75% | 75-100% |
| 464 | 75-100% | 75-100% | 75-100% | 75-100% | 75-100% | 0-25% |
| 465 | 0-25% | 0-25% | 0-25% | 0-25% | 0-25% | 75-100% |
| 466 | 75-100% | 75-100% | 75-100% | 75-100% | 25-75% | 75-100% |
| 467 | 25-75% | 25-75% | 25-75% | 25-75% | 25-75% | 75-100% |
| 468 | 0-25% | 25-75% | 25-75% | 25-75% | 25-75% | 75-100% |
| 469 | 75-100% | 75-100% | 75-100% | 75-100% | 25-75% | 75-100% |
| 470 | 0-25% | 0-25% | 0-25% | 0-25% | 0-25% | 75-100% |
| 471 | 75-100% | 75-100% | 25-75% | 75-100% | 75-100% | 75-100% |
| 472 | 75-100% | 75-100% | 75-100% | 75-100% | 25-75% | 75-100% |
| 473 | 75-100% | 75-100% | 75-100% | 75-100% | 75-100% | 75-100% |
| 474 | 75-100% | 75-100% | 75-100% | 75-100% | 75-100% | 75-100% |
| 475 | 75-100% | 75-100% | 75-100% | 75-100% | 75-100% | 75-100% |
| 476 | 75-100% | 75-100% | 75-100% | 75-100% | 75-100% | 75-100% |
| 477 | 0-25% | 25-75% | 0-25% | 25-75% | 25-75% | 75-100% |
| 478 | 25-75% | 25-75% | 0-25% | 25-75% | 25-75% | 75-100% |
| 479 | 0-25% | 25-75% | 0-25% | 0-25% | 75-100% | 75-100% |
| 480 | 75-100% | 75-100% | 75-100% | 75-100% | 75-100% | 75-100% |
| 481 | 25-75% | 25-75% | 25-75% | 25-75% | 25-75% | 75-100% |
| 482 | 25-75% | 25-75% | 25-75% | 25-75% | 25-75% | 75-100% |
| 483 | 25-75% | 25-75% | 25-75% | 25-75% | 25-75% | 75-100% |
| 484 | 0-25% | 25-75% | 0-25% | 0-25% | 0-25% | 75-100% |
| 485 | 0-25% | 0-25% | 0-25% | 0-25% | 0-25% | 75-100% |
| 486 | 0-25% | 0-25% | 0-25% | 0-25% | 0-25% | 75-100% |
| 487 | 75-100% | 75-100% | 0-25% | 25-75% | 75-100% | 75-100% |
| 488 | 75-100% | 75-100% | 0-25% | 25-75% | 25-75% | 75-100% |
| 489 | 25-75% | 25-75% | 25-75% | 25-75% | 25-75% | 75-100% |
| 490 | 75-100% | 75-100% | 75-100% | 75-100% | 75-100% | 75-100% |
| 491 | 0-25% | 0-25% | 0-25% | 0-25% | 0-25% | 75-100% |
| 492 | 0-25% | 0-25% | 0-25% | 0-25% | 0-25% | 75-100% |
| 493 | 75-100% | 75-100% | 75-100% | 75-100% | 25-75% | 75-100% |
| 494 | 0-25% | 75-100% | 75-100% | 75-100% | 75-100% | 0-25% |
| 495 | 0-25% | 75-100% | 75-100% | 75-100% | 75-100% | 75-100% |
| 496 | 25-75% | 25-75% | 0-25% | 25-75% | 25-75% | 0-25% |
| 497 | 75-100% | 75-100% | 75-100% | 75-100% | 75-100% | 75-100% |
| 498 | 75-100% | 75-100% | 75-100% | 75-100% | 0-25% | 75-100% |
| 499 | 0-25% | 75-100% | 75-100% | 75-100% | 75-100% | 75-100% |
| 500 | 75-100% | 75-100% | 75-100% | 75-100% | 25-75% | 75-100% |
| 501 | 75-100% | 25-75% | 25-75% | 25-75% | 25-75% | 0-25% |
| 502 | 25-75% | 25-75% | 25-75% | 25-75% | 75-100% | 0-25% |
| 503 | 75-100% | 25-75% | 75-100% | 75-100% | 75-100% | 75-100% |
| 504 | 75-100% | 75-100% | 75-100% | 75-100% | 75-100% | 75-100% |
| 505 | 0-25% | 75-100% | 0-25% | 0-25% | 0-25% | ND |
| 506 | 0-25% | 75-100% | 75-100% | 75-100% | 75-100% | 75-100% |
| 507 | 75-100% | 75-100% | 75-100% | 75-100% | 75-100% | 0-25% |
| 508 | 0-25% | 75-100% | 0-25% | 0-25% | 0-25% | ND |
| 509 | 75-100% | 75-100% | 75-100% | 75-100% | 75-100% | 0-25% |
| 510 | 75-100% | 75-100% | 75-100% | 75-100% | 75-100% | 0-25% |
| 511 | 75-100% | 75-100% | 75-100% | 75-100% | 75-100% | 75-100% |
| 512 | 0-25% | 75-100% | 75-100% | 25-75% | 25-75% | 75-100% |
| 513 | 75-100% | 75-100% | 75-100% | 75-100% | 75-100% | 75-100% |
| 514 | 75-100% | 75-100% | 75-100% | 75-100% | 25-75% | 75-100% |
| 515 | 75-100% | 75-100% | 75-100% | 75-100% | 75-100% | 75-100% |
| 516 | 0-25% | 75-100% | 75-100% | 75-100% | 75-100% | 75-100% |
| 517 | 75-100% | 75-100% | 75-100% | 75-100% | 25-75% | 75-100% |
| 518 | 25-75% | 25-75% | 25-75% | 25-75% | 25-75% | 25-75% |
| 519 | 25-75% | 0-25% | 25-75% | 25-75% | 75-100% | 0-25% |
| 520 | 0-25% | 75-100% | 75-100% | 75-100% | 75-100% | 75-100% |
| 521 | 0-25% | 75-100% | 75-100% | 75-100% | 75-100% | 0-25% |
| 522 | 75-100% | 75-100% | 75-100% | 75-100% | 75-100% | 0-25% |
| 523 | 75-100% | 75-100% | 25-75% | 75-100% | 75-100% | 75-100% |
| 524 | 75-100% | 75-100% | 75-100% | 75-100% | 25-75% | 75-100% |
| 525 | 0-25% | 0-25% | 75-100% | 75-100% | 75-100% | 75-100% |
| 526 | 0-25% | 75-100% | 75-100% | 75-100% | 75-100% | 75-100% |
| 527 | 0-25% | 0-25% | 0-25% | 0-25% | 0-25% | 75-100% |
| 528 | 75-100% | 75-100% | 75-100% | 75-100% | 75-100% | 75-100% |
| 529 | 75-100% | 25-75% | 75-100% | 75-100% | 75-100% | 75-100% |
| 530 | 75-100% | 75-100% | 75-100% | 75-100% | 25-75% | 75-100% |
| 531 | 25-75% | 25-75% | 25-75% | 25-75% | 25-75% | 75-100% |
| 532 | 0-25% | 75-100% | 75-100% | 75-100% | 75-100% | 0-25% |
| 533 | 0-25% | 75-100% | 75-100% | 75-100% | 75-100% | 75-100% |
| 534 | 75-100% | 75-100% | 75-100% | 75-100% | 75-100% | 75-100% |
| 535 | 75-100% | 75-100% | ND | 75-100% | 75-100% | 75-100% |
| 536 | 0-25% | 75-100% | 75-100% | 75-100% | 75-100% | 75-100% |
| 537 | 0-25% | 75-100% | 0-25% | 0-25% | 0-25% | 75-100% |
| 538 | 0-25% | 0-25% | 0-25% | 25-75% | 0-25% | 0-25% |
| 539 | 0-25% | 75-100% | 75-100% | 75-100% | 75-100% | 75-100% |
| 540 | 75-100% | 0-25% | 75-100% | 75-100% | 75-100% | 75-100% |
| 541 | 0-25% | 25-75% | 25-75% | 25-75% | 25-75% | 0-25% |
| 542 | 0-25% | 25-75% | 25-75% | 25-75% | 25-75% | 0-25% |
| 543 | 75-100% | 75-100% | 75-100% | 75-100% | 75-100% | 0-25% |
| 544 | 75-100% | 75-100% | 75-100% | 75-100% | 25-75% | 75-100% |
| 545 | 0-25% | 0-25% | 25-75% | ND | 0-25% | 10-25% |
| 546 | 0-25% | 75-100% | 25-75% | 0-25% | 0-25% | 75-100% |
| 547 | 75-100% | 75-100% | 75-100% | 75-100% | 25-75% | 75-100% |
| 548 | 75-100% | 25-75% | 75-100% | 75-100% | 75-100% | 75-100% |
| 549 | 25-75% | 75-100% | 75-100% | 75-100% | 75-100% | 75-100% |
| 550 | 0-25% | 75-100% | 75-100% | 75-100% | 75-100% | 75-100% |
| 551 | 0-25% | 75-100% | 75-100% | 75-100% | 75-100% | 75-100% |
| 552 | 75-100% | 75-100% | 75-100% | 75-100% | 75-100% | 75-100% |
| 553 | 75-100% | 25-75% | 75-100% | 75-100% | 75-100% | |
| 554 | 75-100% | 25-75% | 75-100% | 75-100% | 75-100% | 75-100% |
| 555 | 0-25% | 0-25% | 0-25% | 0-25% | 0-25% | 0-25% |
| 556 | 0-25% | 25-75% | 75-100% | 75-100% | 75-100% | 75-100% |
| 557 | 25-75% | 25-75% | 25-75% | 25-75% | 25-75% | 75-100% |
| 558 | 0-25% | 0-25% | 0-25% | 0-25% | 0-25% | 75-100% |
| 559 | 0-25% | 75-100% | 75-100% | 75-100% | 75-100% | 75-100% |
| 560 | 75-100% | 75-100% | 75-100% | 75-100% | 75-100% | 0-25% |
| 561 | 0-25% | 25-75% | 25-75% | 25-75% | 25-75% | 75-100% |
| 562 | 75-100% | 75-100% | 75-100% | 75-100% | 75-100% | 75-100% |
| 563 | 75-100% | 75-100% | 75-100% | 75-100% | 75-100% | 75-100% |
| 564 | 0-25% | 75-100% | 75-100% | 75-100% | 25-75% | 75-100% |
| 565 | 0-25% | 75-100% | 75-100% | 75-100% | 75-100% | 0-25% |
| 566 | 0-25% | 25-75% | 25-75% | 25-75% | 25-75% | ND |
| 567 | 75-100% | 75-100% | 75-100% | 75-100% | 75-100% | 75-100% |
| 568 | 0-25% | 25-75% | 0-25% | 0-25% | 0-25% | 0-25% |
| 569 | 75-100% | 75-100% | 75-100% | 75-100% | 25-75% | 75-100% |
| 570 | 0-25% | 75-100% | 75-100% | 75-100% | 75-100% | 0-25% |
| 571 | 0-25% | 25-75% | 25-75% | 25-75% | 25-75% | 75-100% |
| 572 | 75-100% | 25-75% | 75-100% | 25-75% | 25-75% | 75-100% |
| 573 | 75-100% | 75-100% | 75-100% | 75-100% | 25-75% | 0-25% |
| 574 | 75-100% | 75-100% | 75-100% | 75-100% | 75-100% | 75-100% |
| 575 | 0-25% | 75-100% | 75-100% | 75-100% | 75-100% | 75-100% |
| 576 | 75-100% | 0-25% | 75-100% | 0-25% | 0-25% | 0-25% |
| 577 | 75-100% | 75-100% | 75-100% | 75-100% | 25-75% | 75-100% |
| 578 | 75-100% | 75-100% | 75-100% | 75-100% | 75-100% | 0-25% |
| 579 | 0-25% | 0-25% | 0-25% | 0-25% | 0-25% | 0-25% |
| 580 | 75-100% | 75-100% | 75-100% | 75-100% | 25-75% | 0-25% |
| 581 | 75-100% | 75-100% | 75-100% | 75-100% | 75-100% | 0-25% |
| 582 | 75-100% | 25-75% | 75-100% | 75-100% | 75-100% | 75-100% |
| 583 | 75-100% | 25-75% | 75-100% | 75-100% | 75-100% | 75-100% |
| 584 | 0-25% | 0-25% | 0-25% | 0-25% | 25-75% | 75-100% |
| 585 | 75-100% | 75-100% | 75-100% | 75-100% | 25-75% | 75-100% |
| 586 | 75-100% | 75-100% | 75-100% | 75-100% | 75-100% | 75-100% |
| 587 | 0-25% | 75-100% | 75-100% | 75-100% | 75-100% | 75-100% |
| 588 | 0-25% | 25-75% | 0-25% | 0-25% | 0-25% | 0-25% |
| 589 | 75-100% | 0-25% | 0-25% | 0-25% | 0-25% | 0-25% |
| 590 | 0-25% | 75-100% | 75-100% | 0-25% | 0-25% | 0-25% |
| 591 | 0-25% | 75-100% | 0-25% | 0-25% | 0-25% | 0-25% |
| 592 | 0-25% | 75-100% | 75-100% | 75-100% | 25-75% | 75-100% |
| 593 | 25-75% | 25-75% | 25-75% | 0-25% | 0-25% | 0-25% |
| 594 | 75-100% | 75-100% | 75-100% | 75-100% | 25-75% | 75-100% |
| 595 | 75-100% | 75-100% | 75-100% | 75-100% | 75-100% | 75-100% |
| 596 | 75-100% | 75-100% | 0-25% | 25-75% | 75-100% | 75-100% |
| 597 | 0-25% | 0-25% | 0-25% | 0-25% | 0-25% | 75-100% |
| 598 | 75-100% | 75-100% | 75-100% | 75-100% | 75-100% | 75-100% |
| 599 | 75-100% | 25-75% | 75-100% | 75-100% | 25-75% | 75-100% |
| 600 | 0-25% | 75-100% | 75-100% | 75-100% | 25-75% | 75-100% |
| 601 | 75-100% | 75-100% | 75-100% | 75-100% | 75-100% | 75-100% |
| 602 | 0-25% | 75-100% | 75-100% | 75-100% | 75-100% | 75-100% |
| 603 | 0-25% | 75-100% | 75-100% | 75-100% | 75-100% | 75-100% |
| 604 | 25-75% | 75-100% | 75-100% | 75-100% | 75-100% | 75-100% |
| 605 | 25-75% | 75-100% | 75-100% | 75-100% | 75-100% | 75-100% |
| 606 | 75-100% | 25-75% | 75-100% | 75-100% | 75-100% | 75-100% |
| 607 | 75-100% | 75-100% | 75-100% | 75-100% | 75-100% | 75-100% |
| 608 | 0-25% | 75-100% | 75-100% | 75-100% | 75-100% | 75-100% |
| 609 | 75-100% | 75-100% | 0-25% | 0-25% | 75-100% | 75-100% |
| 610 | 75-100% | 75-100% | 75-100% | 75-100% | 75-100% | 75-100% |
| 611 | 75-100% | 75-100% | 75-100% | 75-100% | 75-100% | 75-100% |
| 612 | 75-100% | 75-100% | 75-100% | 75-100% | 25-75% | 75-100% |
| 613 | 75-100% | 75-100% | 0-25% | 0-25% | 0-25% | ND |
| 614 | 75-100% | 25-75% | 25-75% | 25-75% | 25-75% | 75-100% |
| 615 | 75-100% | 75-100% | 75-100% | 75-100% | 75-100% | 75-100% |
| 616 | 25-75% | 75-100% | 25-75% | 25-75% | 25-75% | 75-100% |
| 617 | 75-100% | 75-100% | 75-100% | 75-100% | 25-75% | 75-100% |
| 618 | 75-100% | 75-100% | 0-25% | 75-100% | 75-100% | 75-100% |
| 619 | 0-25% | 0-25% | 0-25% | 0-25% | 0-25% | 75-100% |
| 620 | 75-100% | 25-75% | 75-100% | 75-100% | 75-100% | 75-100% |
| 621 | 75-100% | 75-100% | 75-100% | 75-100% | 25-75% | 75-100% |
| 622 | 25-75% | 75-100% | 75-100% | 75-100% | 0-25% | 75-100% |
| 623 | 0-25% | 0-25% | 0-25% | 0-25% | 0-25% | 75-100% |
| 624 | 75-100% | 75-100% | 75-100% | 75-100% | 25-75% | 75-100% |
| 625 | 0-25% | 75-100% | 75-100% | 75-100% | 75-100% | 75-100% |
| 626 | 75-100% | 75-100% | 75-100% | 25-75% | 25-75% | 0-25% |
| 627 | 0-25% | 75-100% | 75-100% | 75-100% | 25-75% | 75-100% |
| 628 | 0-25% | 75-100% | 75-100% | 25-75% | 25-75% | 75-100% |
| 629 | 25-75% | 25-75% | 25-75% | ND | 75-100% | 75-100% |
| 630 | 75-100% | 75-100% | 75-100% | 75-100% | 75-100% | 75-100% |
| 631 | 75-100% | 75-100% | 75-100% | 75-100% | 25-75% | 75-100% |
| 632 | 25-75% | 25-75% | 25-75% | 25-75% | 25-75% | 75-100% |
| 633 | 75-100% | 0-25% | 0-25% | 0-25% | 0-25% | 75-100% |
| 634 | 25-75% | 25-75% | 25-75% | 25-75% | 25-75% | 75-100% |
| 635 | 0-25% | 75-100% | 75-100% | 75-100% | 75-100% | 75-100% |
| 636 | 75-100% | 75-100% | 75-100% | 75-100% | 75-100% | 75-100% |
| 637 | 0-25% | 0-25% | 0-25% | 0-25% | 0-25% | 75-100% |
| 638 | 25-75% | 75-100% | 75-100% | 75-100% | 75-100% | 75-100% |
| 639 | 0-25% | 75-100% | 75-100% | 75-100% | 75-100% | 75-100% |
| 640 | 25-75% | 75-100% | 25-75% | 75-100% | 75-100% | 75-100% |
| 641 | 75-100% | 75-100% | 75-100% | 75-100% | 75-100% | 0-25% |
| 642 | 75-100% | 75-100% | 75-100% | 75-100% | 75-100% | 75-100% |
| 643 | 75-100% | 75-100% | 75-100% | 75-100% | 75-100% | 75-100% |
| 644 | 75-100% | 75-100% | 0-25% | 25-75% | 25-75% | 75-100% |
| 645 | 75-100% | 75-100% | 75-100% | 75-100% | 75-100% | 75-100% |
| 646 | 75-100% | 75-100% | 75-100% | 75-100% | 75-100% | 75-100% |
| 647 | 75-100% | 75-100% | 75-100% | 75-100% | 25-75% | 75-100% |
| 648 | 75-100% | 75-100% | 75-100% | 75-100% | 75-100% | 75-100% |
| 649 | 0-25% | 75-100% | 75-100% | 75-100% | 75-100% | |
| 650 | 0-25% | 75-100% | 75-100% | 75-100% | 25-75% | 75-100% |
| 651 | 25-75% | 25-75% | 25-75% | 25-75% | 25-75% | 75-100% |
| 652 | 75-100% | 75-100% | 75-100% | 75-100% | 75-100% | 75-100% |
| 653 | 75-100% | 75-100% | 75-100% | 75-100% | 75-100% | 75-100% |
| 654 | 75-100% | 75-100% | 75-100% | 75-100% | 75-100% | 75-100% |
| 655 | 75-100% | 75-100% | 75-100% | 75-100% | 25-75% | 75-100% |
| 656 | 75-100% | 75-100% | 0-25% | 75-100% | 75-100% | 75-100% |
| 657 | 75-100% | 75-100% | 75-100% | 75-100% | 25-75% | 75-100% |
| 658 | 75-100% | 75-100% | 75-100% | ND | 25-75% | ND |
| 659 | 25-75% | 25-75% | 25-75% | 25-75% | 25-75% | 75-100% |
| 660 | 75-100% | 75-100% | 75-100% | 75-100% | 75-100% | 75-100% |
| 661 | 75-100% | 25-75% | 75-100% | 75-100% | 75-100% | 75-100% |
| 662 | 0-25% | 75-100% | 75-100% | 75-100% | 75-100% | 75-100% |
| 663 | 0-25% | 75-100% | 75-100% | 75-100% | 75-100% | 75-100% |
| 664 | 75-100% | 75-100% | 75-100% | 75-100% | 0-25% | ND |
| 665 | 25-75% | 25-75% | 25-75% | 25-75% | 25-75% | 0-25% |
| 666 | 0-25% | 75-100% | 75-100% | 75-100% | 75-100% | 75-100% |
| 667 | 0-25% | 75-100% | 75-100% | 75-100% | 75-100% | 75-100% |
| 668 | 75-100% | 75-100% | 75-100% | 75-100% | 25-75% | 75-100% |
| 669 | 0-25% | 25-75% | 0-25% | 0-25% | 25-75% | 0-25% |
| 670 | 0-25% | 75-100% | 75-100% | 75-100% | 75-100% | 75-100% |
| 671 | 75-100% | 75-100% | 0-25% | 75-100% | 75-100% | 75-100% |
| 672 | 75-100% | 75-100% | 0-25% | 75-100% | 75-100% | 75-100% |
| 673 | 25-75% | 75-100% | 25-75% | 25-75% | 0-25% | ND |
| 674 | 75-100% | 75-100% | 25-75% | 75-100% | 75-100% | 75-100% |
| 675 | 25-75% | 25-75% | 25-75% | 25-75% | 25-75% | 75-100% |
| 676 | 0-25% | 0-25% | 0-25% | 0-25% | 0-25% | 75-100% |
| 677 | 75-100% | 0-25% | 0-25% | 0-25% | 0-25% | ND |
| 678 | 75-100% | 75-100% | 75-100% | 75-100% | 25-75% | 75-100% |
| 679 | 75-100% | 75-100% | 75-100% | 75-100% | 75-100% | 75-100% |
| 680 | 25-75% | 75-100% | 25-75% | 25-75% | 25-75% | 25-75% |
| 681 | 0-25% | 0-25% | 75-100% | 0-25% | 0-25% | 75-100% |
| 682 | 0-25% | 25-75% | 0-25% | 0-25% | 25-75% | 75-100% |
| 683 | 75-100% | 75-100% | 75-100% | 75-100% | 75-100% | 75-100% |
| 684 | 0-25% | 25-75% | 0-25% | 0-25% | 0-25% | 0-25% |
| 685 | 25-75% | 25-75% | 25-75% | 25-75% | 25-75% | 75-100% |
| 686 | 75-100% | 75-100% | 75-100% | 75-100% | 25-75% | 75-100% |
| 687 | 75-100% | 25-75% | 25-75% | 25-75% | 25-75% | 75-100% |
| 688 | 25-75% | 25-75% | 25-75% | 25-75% | 25-75% | ND |
| 689 | 0-25% | 75-100% | 75-100% | 75-100% | 75-100% | 0-25% |
| 690 | 75-100% | 75-100% | 75-100% | 75-100% | 25-75% | 75-100% |
| 691 | 0-25% | 0-25% | 0-25% | 0-25% | 0-25% | 0-25% |
| 692 | 0-25% | 75-100% | 75-100% | 75-100% | 75-100% | 75-100% |
| 693 | 75-100% | 25-75% | 75-100% | 75-100% | 75-100% | 75-100% |
| 694 | 75-100% | 75-100% | 75-100% | 75-100% | 75-100% | 0-25% |
| 695 | 75-100% | 25-75% | 75-100% | 75-100% | 75-100% | 75-100% |
| 696 | 75-100% | 25-75% | 25-75% | 25-75% | 0-25% | 0-25% |
| 697 | 75-100% | 75-100% | 75-100% | 75-100% | 75-100% | 75-100% |
| 698 | 75-100% | 75-100% | 75-100% | 75-100% | 75-100% | 75-100% |
| 699 | 75-100% | 75-100% | 75-100% | ND | 75-100% | 75-100% |
| 700 | 0-25% | 25-75% | 0-25% | 0-25% | 25-75% | 75-100% |
| 701 | 75-100% | 0-25% | 75-100% | 75-100% | 75-100% | 75-100% |
| 702 | 0-25% | 75-100% | 75-100% | 75-100% | 75-100% | 75-100% |
| 703 | 0-25% | 75-100% | 75-100% | 75-100% | 75-100% | 75-100% |
| 704 | 25-75% | 25-75% | 25-75% | 25-75% | 75-100% | 75-100% |
| 705 | 75-100% | 25-75% | 75-100% | 75-100% | 75-100% | 75-100% |
| 706 | 25-75% | 75-100% | 25-75% | 25-75% | 25-75% | 75-100% |
| 707 | 0-25% | 75-100% | 75-100% | 75-100% | 75-100% | 75-100% |
| 708 | 75-100% | 75-100% | 75-100% | 75-100% | 75-100% | 75-100% |
| 709 | 0-25% | 75-100% | 75-100% | 75-100% | 75-100% | 75-100% |
| 710 | 75-100% | 75-100% | 75-100% | 75-100% | 75-100% | 75-100% |
| 711 | 75-100% | 25-75% | 75-100% | 75-100% | 75-100% | 75-100% |
| 712 | 25-75% | 75-100% | 75-100% | 75-100% | 75-100% | 75-100% |

ND = not determined

TABLE 7
Overview of the genomic regions and ranges of methylation within which the actual value of
methylation of said genomic regions lies specific of the said cells, tissues and/or organs.
The actual value of methylation is shown in FIGS. 1.1-1.403.

| SEQ | | | | | | |
|---|---|---|---|---|---|---|
| ID | CD4 | CD8 | | Embryonic | | |
| NO: | T- | T- | Embryonic | Skeletal | | Heart |
| Genomic | lymphocyte | lymphocyte | Liver | Muscle | Fibroblast | Muscle |
| 713 | 75-100% | 75-100% | 75-100% | 75-100% | 75-100% | 75-100% |
| 714 | 25-75% | 25-75% | ND | 0-25% | 0-25% | 25-75% |
| 715 | 25-75% | 25-75% | 0-25% | 0-25% | 0-25% | 0-25% |
| 716 | 75-100% | 75-100% | 75-100% | 75-100% | 75-100% | 75-100% |
| 717 | 75-100% | 75-100% | 25-75% | 25-75% | 25-75% | 25-75% |
| 718 | 75-100% | 75-100% | 75-100% | 75-100% | 0-25% | 75-100% |
| 719 | 25-75% | 25-75% | 25-75% | 0-25% | 0-25% | 25-75% |
| 720 | 0-25% | 0-25% | 0-25% | 75-100% | 75-100% | 0-25% |
| 721 | 75-100% | 75-100% | 75-100% | 75-100% | 0-25% | 75-100% |
| 722 | 75-100% | 75-100% | 75-100% | 75-100% | 25-75% | 75-100% |
| 723 | 75-100% | 75-100% | 75-100% | 0-25% | 0-25% | 75-100% |
| 724 | 0-25% | 0-25% | 75-100% | 75-100% | 75-100% | 75-100% |
| 725 | 75-100% | 75-100% | 75-100% | 75-100% | 25-75% | 25-75% |
| 726 | 0-25% | 0-25% | 25-75% | 0-25% | 0-25% | 25-75% |
| 727 | 25-75% | 25-75% | 25-75% | 25-75% | 25-75% | 25-75% |
| 728 | 75-100% | 75-100% | 75-100% | 75-100% | 0-25% | 75-100% |
| 729 | 75-100% | 75-100% | 75-100% | 75-100% | 25-75% | 75-100% |
| 730 | 25-75% | 75-100% | 75-100% | 75-100% | 75-100% | 75-100% |
| 731 | 75-100% | 75-100% | 75-100% | 75-100% | 0-25% | 75-100% |
| 732 | 75-100% | 75-100% | 75-100% | 75-100% | 25-75% | 75-100% |
| 733 | 75-100% | 75-100% | 25-75% | 25-75% | 25-75% | 25-75% |
| 734 | 25-75% | 25-75% | 25-75% | 25-75% | 25-75% | 25-75% |
| 735 | 75-100% | 75-100% | 25-75% | 25-75% | 25-75% | 25-75% |
| 736 | 75-100% | 75-100% | 75-100% | 75-100% | 0-25% | 75-100% |
| 737 | 75-100% | 75-100% | 75-100% | 25-75% | 25-75% | 25-75% |
| 738 | 0-25% | 0-25% | 0-25% | 0-25% | 25-75% | 25-75% |
| 739 | 75-100% | 75-100% | 75-100% | 75-100% | 25-75% | 75-100% |
| 740 | 75-100% | 75-100% | 75-100% | 75-100% | 25-75% | 75-100% |
| 741 | 75-100% | 75-100% | 75-100% | 75-100% | 0-25% | 75-100% |
| 742 | 75-100% | 75-100% | 75-100% | 25-75% | 25-75% | 25-75% |
| 743 | 75-100% | 75-100% | 75-100% | 75-100% | 75-100% | 75-100% |
| 744 | 75-100% | 75-100% | 75-100% | 25-75% | 25-75% | 25-75% |
| 745 | 75-100% | 75-100% | 75-100% | 75-100% | 75-100% | 75-100% |
| 746 | 75-100% | 75-100% | 75-100% | 0-25% | 0-25% | 25-75% |
| 747 | 75-100% | 75-100% | 75-100% | 75-100% | 25-75% | 75-100% |
| 748 | 75-100% | 75-100% | 75-100% | 25-75% | 75-100% | 75-100% |
| 749 | 75-100% | 75-100% | 75-100% | 25-75% | 25-75% | 25-75% |
| 750 | 75-100% | 75-100% | 25-75% | 25-75% | 0-25% | 0-25% |
| 751 | 75-100% | 75-100% | 75-100% | 75-100% | 0-25% | 75-100% |
| 752 | 75-100% | 75-100% | 75-100% | 75-100% | 75-100% | 25-75% |
| 753 | 75-100% | 75-100% | 75-100% | ND | 0-25% | 75-100% |
| 754 | 75-100% | 75-100% | 25-75% | 25-75% | 25-75% | 75-100% |
| 755 | 25-75% | 25-75% | 25-75% | 25-75% | 25-75% | 25-75% |
| 756 | 75-100% | 75-100% | 75-100% | 75-100% | 0-25% | 75-100% |
| 757 | 75-100% | 75-100% | 75-100% | 25-75% | 25-75% | 25-75% |
| 758 | 75-100% | 75-100% | 75-100% | 75-100% | 25-75% | 75-100% |
| 759 | 75-100% | 75-100% | 75-100% | 75-100% | 75-100% | 25-75% |
| 760 | 0-25% | 0-25% | 25-75% | 25-75% | 25-75% | 25-75% |
| 761 | 75-100% | 75-100% | 75-100% | 75-100% | 0-25% | 75-100% |
| 762 | 0-25% | ND | 0-25% | 75-100% | 75-100% | 75-100% |
| 763 | 75-100% | 75-100% | 25-75% | 75-100% | 75-100% | 75-100% |
| 764 | 75-100% | 75-100% | 75-100% | 25-75% | 25-75% | 25-75% |
| 765 | 0-25% | 0-25% | 0-25% | 25-75% | 25-75% | 25-75% |
| 766 | 0-25% | 0-25% | 25-75% | 75-100% | 75-100% | 75-100% |
| 767 | 25-75% | 25-75% | 25-75% | 0-25% | 0-25% | 0-25% |
| 768 | 25-75% | 25-75% | 0-25% | 25-75% | 25-75% | 25-75% |
| 769 | 75-100% | 75-100% | 75-100% | 25-75% | 0-25% | 25-75% |
| 770 | 75-100% | 75-100% | 75-100% | 75-100% | 25-75% | 75-100% |
| 771 | 75-100% | 75-100% | 75-100% | 75-100% | 75-100% | 75-100% |
| 772 | 75-100% | 75-100% | 75-100% | 25-75% | 25-75% | 75-100% |
| 773 | 75-100% | 75-100% | 75-100% | 75-100% | 0-25% | 75-100% |
| 774 | 75-100% | 75-100% | 75-100% | 75-100% | 25-75% | 75-100% |
| 775 | 75-100% | 75-100% | 75-100% | 75-100% | 0-25% | 75-100% |
| 776 | 75-100% | 75-100% | 75-100% | 75-100% | 75-100% | 25-75% |
| 777 | 75-100% | 75-100% | 75-100% | 75-100% | 25-75% | 75-100% |
| 778 | 0-25% | 0-25% | 75-100% | 75-100% | 75-100% | 75-100% |
| 779 | 75-100% | 75-100% | 75-100% | ND | 25-75% | 25-75% |
| 780 | 75-100% | 75-100% | 75-100% | 25-75% | 25-75% | 25-75% |
| 781 | 75-100% | 75-100% | 75-100% | 25-75% | 25-75% | 75-100% |
| 782 | 75-100% | 75-100% | 75-100% | 75-100% | 0-25% | 75-100% |
| 783 | 75-100% | 75-100% | 75-100% | 75-100% | 75-100% | 75-100% |
| 784 | 75-100% | 75-100% | 75-100% | 75-100% | 25-75% | 25-75% |
| 785 | 25-75% | 25-75% | 25-75% | 25-75% | 0-25% | 0-25% |
| 786 | 75-100% | 75-100% | 75-100% | 25-75% | 25-75% | 25-75% |
| 787 | 0-25% | 0-25% | 0-25% | 0-25% | 0-25% | 0-25% |
| 788 | 75-100% | 75-100% | 75-100% | 75-100% | 25-75% | 75-100% |
| 789 | 75-100% | 75-100% | 75-100% | 75-100% | 0-25% | 75-100% |
| 790 | 25-75% | 25-75% | 25-75% | 25-75% | 25-75% | 25-75% |
| 791 | 0-25% | 0-25% | 0-25% | 0-25% | 75-100% | 25-75% |
| 792 | 75-100% | 75-100% | 75-100% | 25-75% | 25-75% | 75-100% |
| 793 | 75-100% | 75-100% | 75-100% | 75-100% | 75-100% | 75-100% |
| 794 | 75-100% | 75-100% | 75-100% | 75-100% | 75-100% | 25-75% |
| 795 | 75-100% | 75-100% | 25-75% | 25-75% | 25-75% | 25-75% |
| 796 | 75-100% | 75-100% | 75-100% | 0-25% | 0-25% | 25-75% |
| 797 | 75-100% | 75-100% | 75-100% | 75-100% | 75-100% | 75-100% |
| 798 | 75-100% | 75-100% | 75-100% | 75-100% | 75-100% | 75-100% |
| 799 | 75-100% | 75-100% | 75-100% | 0-25% | 0-25% | 0-25% |
| 800 | 75-100% | 75-100% | 75-100% | 75-100% | 25-75% | 75-100% |
| 801 | 75-100% | 75-100% | 75-100% | 25-75% | 25-75% | 25-75% |
| 802 | 75-100% | 75-100% | 75-100% | 75-100% | 75-100% | 75-100% |
| 803 | 75-100% | 75-100% | 75-100% | ND | 0-25% | 25-75% |
| 804 | 25-75% | 25-75% | 25-75% | 25-75% | 25-75% | 75-100% |
| 805 | 75-100% | 75-100% | 75-100% | 75-100% | 25-75% | 75-100% |
| 806 | 75-100% | 75-100% | 75-100% | 25-75% | 25-75% | 25-75% |
| 807 | 75-100% | 75-100% | 75-100% | 75-100% | 75-100% | 75-100% |
| 808 | 75-100% | 75-100% | 75-100% | 25-75% | 25-75% | 25-75% |
| 809 | 75-100% | 75-100% | 75-100% | 75-100% | 0-25% | 75-100% |
| 810 | 75-100% | 75-100% | 75-100% | 75-100% | 75-100% | 25-75% |
| 811 | 75-100% | 75-100% | 75-100% | 75-100% | 25-75% | 75-100% |
| 812 | 75-100% | 75-100% | 75-100% | 25-75% | 25-75% | 75-100% |
| 813 | 75-100% | 75-100% | 75-100% | 75-100% | 25-75% | 75-100% |
| 814 | 75-100% | 75-100% | 75-100% | 75-100% | 5β | 75-100% |
| 815 | 75-100% | 75-100% | 75-100% | 25-75% | 25-75% | 25-75% |
| 816 | 75-100% | 75-100% | 75-100% | 75-100% | 75-100% | 75-100% |
| 817 | 75-100% | 75-100% | 75-100% | 25-75% | 25-75% | 75-100% |
| 818 | 75-100% | 75-100% | 75-100% | 75-100% | 0-25% | 75-100% |
| 819 | 75-100% | 75-100% | 75-100% | 25-75% | 25-75% | 75-100% |
| 820 | 75-100% | 75-100% | 75-100% | 75-100% | 25-75% | 75-100% |
| 821 | 75-100% | 75-100% | 75-100% | 75-100% | 25-75% | 75-100% |
| 822 | 25-75% | 25-75% | 25-75% | 25-75% | 75-100% | 25-75% |
| 823 | 75-100% | 75-100% | 75-100% | 75-100% | 25-75% | 75-100% |
| 824 | 75-100% | 75-100% | 75-100% | 75-100% | 75-100% | 25-75% |

| SEQ | | | | | | |
|---|---|---|---|---|---|---|
| ID | | | | | | |
| NO: | | | | | Skeletal | |
| Genomic | Keratinocyte | Liver | Melanocyte | Placenta | Muscle | Sperm |
| 713 | 75-100% | 25-75% | 75-100% | 75-100% | 25-75% | 25-75% |
| 714 | 0-25% | 25-75% | 0-25% | 25-75% | 25-75% | 25-75% |
| 715 | 0-25% | 25-75% | 0-25% | 0-25% | 0-25% | 0-25% |
| 716 | 25-75% | 25-75% | 75-100% | 25-75% | 25-75% | 0-25% |
| 717 | 25-75% | 75-100% | 0-25% | 25-75% | 25-75% | 0-25% |
| 718 | 75-100% | 75-100% | 0-25% | 75-100% | 75-100% | 75-100% |
| 719 | 0-25% | 25-75% | 0-25% | 25-75% | 25-75% | 0-25% |
| 720 | 0-25% | 0-25% | 0-25% | 75-100% | 75-100% | 0-25% |
| 721 | 0-25% | 75-100% | 75-100% | 75-100% | 75-100% | 75-100% |
| 722 | 25-75% | 75-100% | 75-100% | 75-100% | 75-100% | 75-100% |
| 723 | 75-100% | 75-100% | 75-100% | 75-100% | 0-25% | 75-100% |
| 724 | 0-25% | 75-100% | 75-100% | 75-100% | 75-100% | 75-100% |
| 725 | 75-100% | 75-100% | 75-100% | 25-75% | 25-75% | 75-100% |
| 726 | 0-25% | 25-75% | 25-75% | 25-75% | 0-25% | 75-100% |
| 727 | 75-100% | 25-75% | 75-100% | 25-75% | 25-75% | 25-75% |
| 728 | 75-100% | 75-100% | 0-25% | 75-100% | 75-100% | 75-100% |
| 729 | 75-100% | 75-100% | 25-75% | 75-100% | 75-100% | 75-100% |
| 730 | 75-100% | 75-100% | 75-100% | 75-100% | 25-75% | 75-100% |
| 731 | 0-25% | 75-100% | 75-100% | 75-100% | 75-100% | 75-100% |
| 732 | 75-100% | 75-100% | 25-75% | 75-100% | 25-75% | 75-100% |
| 733 | 75-100% | 75-100% | 75-100% | 25-75% | 25-75% | 75-100% |
| 734 | 0-25% | 0-25% | 75-100% | 0-25% | 0-25% | 0-25% |
| 735 | 25-75% | 75-100% | 75-100% | 25-75% | 25-75% | 75-100% |
| 736 | 0-25% | 75-100% | 75-100% | 75-100% | 75-100% | 75-100% |
| 737 | 75-100% | 75-100% | 75-100% | 75-100% | 25-75% | 75-100% |
| 738 | 75-100% | 75-100% | 75-100% | 75-100% | 25-75% | 75-100% |
| 739 | 75-100% | 75-100% | 25-75% | 75-100% | 75-100% | 75-100% |
| 740 | 75-100% | 75-100% | 25-75% | 75-100% | 75-100% | 75-100% |
| 741 | 0-25% | 75-100% | 0-25% | 25-75% | 25-75% | 75-100% |
| 742 | 25-75% | 75-100% | 75-100% | 25-75% | 25-75% | 75-100% |
| 743 | 0-25% | 75-100% | 75-100% | 0-25% | 75-100% | 0-25% |
| 744 | 75-100% | 75-100% | 0-25% | 75-100% | 75-100% | 75-100% |
| 745 | 0-25% | 75-100% | 75-100% | 75-100% | 0-25% | 0-25% |
| 746 | 0-25% | 75-100% | 0-25% | 0-25% | 25-75% | 75-100% |
| 747 | 25-75% | 75-100% | 75-100% | 75-100% | 25-75% | 75-100% |
| 748 | 0-25% | 75-100% | 75-100% | 75-100% | 25-75% | 75-100% |
| 749 | 25-75% | 75-100% | 75-100% | 75-100% | 75-100% | 75-100% |
| 750 | 75-100% | 75-100% | 75-100% | 75-100% | 75-100% | 75-100% |
| 751 | 0-25% | 75-100% | 0-25% | 75-100% | 0-25% | 0-25% |
| 752 | 75-100% | 25-75% | 75-100% | 75-100% | 25-75% | 75-100% |
| 753 | 75-100% | 75-100% | 75-100% | 0-25% | 0-25% | 75-100% |
| 754 | 25-75% | 75-100% | 0-25% | 0-25% | 0-25% | 0-25% |
| 755 | 75-100% | 75-100% | 75-100% | 25-75% | 25-75% | 0-25% |
| 756 | 0-25% | 75-100% | 75-100% | 75-100% | 25-75% | 75-100% |
| 757 | 25-75% | 75-100% | 25-75% | 25-75% | 25-75% | 75-100% |
| 758 | 75-100% | 75-100% | 75-100% | 75-100% | 75-100% | 75-100% |
| 759 | 75-100% | 75-100% | 75-100% | 75-100% | 25-75% | 75-100% |
| 760 | 25-75% | 75-100% | 25-75% | 25-75% | 25-75% | 75-100% |
| 761 | 75-100% | 75-100% | 75-100% | 75-100% | 75-100% | 75-100% |
| 762 | 0-25% | 75-100% | 75-100% | 75-100% | 75-100% | 75-100% |
| 763 | 75-100% | 25-75% | 75-100% | 75-100% | 25-75% | 75-100% |
| 764 | 75-100% | 75-100% | 75-100% | 25-75% | 25-75% | 75-100% |
| 765 | 0-25% | 25-75% | 25-75% | 25-75% | 75-100% | 0-25% |
| 766 | 75-100% | 25-75% | 75-100% | 25-75% | 75-100% | 75-100% |
| 767 | 25-75% | 25-75% | 25-75% | 0-25% | 0-25% | 75-100% |
| 768 | 0-25% | 25-75% | 0-25% | 25-75% | 25-75% | 0-25% |
| 769 | 75-100% | 75-100% | 75-100% | 25-75% | 25-75% | 75-100% |
| 770 | 75-100% | 75-100% | 75-100% | 75-100% | 75-100% | 75-100% |
| 771 | 25-75% | 75-100% | 75-100% | 75-100% | 25-75% | 75-100% |
| 772 | 75-100% | 75-100% | 75-100% | 25-75% | 25-75% | 75-100% |
| 773 | 0-25% | 75-100% | 0-25% | 75-100% | 75-100% | 75-100% |
| 774 | 75-100% | 25-75% | 75-100% | 75-100% | 75-100% | 75-100% |
| 775 | 0-25% | 75-100% | 75-100% | 75-100% | 75-100% | 75-100% |
| 776 | 25-75% | 75-100% | 75-100% | 75-100% | 75-100% | 75-100% |
| 777 | 0-25% | 75-100% | 0-25% | 75-100% | 75-100% | 75-100% |
| 778 | 0-25% | 75-100% | 75-100% | 75-100% | 75-100% | 75-100% |
| 779 | 75-100% | 75-100% | 0-25% | 75-100% | 25-75% | 75-100% |
| 780 | 75-100% | 75-100% | 75-100% | 25-75% | 25-75% | 75-100% |
| 781 | 25-75% | 75-100% | 25-75% | 25-75% | 75-100% | 75-100% |
| 782 | 25-75% | 75-100% | 25-75% | 75-100% | 75-100% | 75-100% |
| 783 | 75-100% | 75-100% | 75-100% | 25-75% | 25-75% | 75-100% |
| 784 | 75-100% | 75-100% | 75-100% | 75-100% | 25-75% | 75-100% |
| 785 | 25-75% | 25-75% | 0-25% | 0-25% | 0-25% | 75-100% |
| 786 | 25-75% | 25-75% | 25-75% | 25-75% | 25-75% | 0-25% |
| 787 | 0-25% | 75-100% | 0-25% | 0-25% | 75-100% | 75-100% |
| 788 | 75-100% | 75-100% | 75-100% | 75-100% | 25-75% | 75-100% |
| 789 | 75-100% | 75-100% | 0-25% | 75-100% | 75-100% | 75-100% |
| 790 | 25-75% | 75-100% | 25-75% | 25-75% | 0-25% | 75-100% |
| 791 | 0-25% | 75-100% | 25-75% | 75-100% | 25-75% | 75-100% |
| 792 | 75-100% | 75-100% | 75-100% | 25-75% | 25-75% | 75-100% |
| 793 | 75-100% | 25-75% | 25-75% | 75-100% | 75-100% | 75-100% |
| 794 | 75-100% | 25-75% | 25-75% | 75-100% | 75-100% | 75-100% |
| 795 | 75-100% | 25-75% | 25-75% | 25-75% | 25-75% | ND |
| 796 | 75-100% | 25-75% | 0-25% | 25-75% | 25-75% | 0-25% |
| 797 | 75-100% | 75-100% | 75-100% | 25-75% | 25-75% | 75-100% |
| 798 | 75-100% | 75-100% | 75-100% | 25-75% | 25-75% | 75-100% |
| 799 | 0-25% | 75-100% | 0-25% | 0-25% | 0-25% | 75-100% |
| 800 | 75-100% | 75-100% | 75-100% | 75-100% | 25-75% | 75-100% |
| 801 | 75-100% | 25-75% | 75-100% | 25-75% | 25-75% | 75-100% |
| 802 | 75-100% | 75-100% | 75-100% | 25-75% | 25-75% | 75-100% |
| 803 | 25-75% | 75-100% | 0-25% | 75-100% | 75-100% | 75-100% |
| 804 | 75-100% | 75-100% | 25-75% | 25-75% | 25-75% | 25-75% |
| 805 | 75-100% | 75-100% | 25-75% | 25-75% | 25-75% | 75-100% |
| 806 | 75-100% | 75-100% | 25-75% | 25-75% | 25-75% | 75-100% |
| 807 | 0-25% | 75-100% | 75-100% | 0-25% | 0-25% | 75-100% |
| 808 | 25-75% | 75-100% | 75-100% | 25-75% | 25-75% | 75-100% |
| 809 | 75-100% | 75-100% | 75-100% | 75-100% | 75-100% | 75-100% |
| 810 | 75-100% | 25-75% | 25-75% | 75-100% | 75-100% | 75-100% |
| 811 | 75-100% | 75-100% | 25-75% | 25-75% | 25-75% | 75-100% |
| 812 | 75-100% | 75-100% | 25-75% | 25-75% | 25-75% | 75-100% |
| 813 | 75-100% | 75-100% | 25-75% | 75-100% | 75-100% | 75-100% |
| 814 | 75-100% | 75-100% | 75-100% | 5β | 25-75% | 75-100% |
| 815 | 75-100% | 75-100% | 25-75% | 25-75% | 25-75% | 75-100% |
| 816 | 25-75% | 75-100% | 75-100% | 25-75% | 25-75% | 75-100% |
| 817 | 75-100% | 75-100% | 75-100% | 25-75% | 25-75% | 75-100% |
| 818 | 75-100% | 75-100% | 75-100% | 75-100% | 0-25% | 75-100% |
| 819 | 75-100% | 75-100% | 25-75% | 25-75% | 25-75% | 75-100% |
| 820 | 0-25% | 75-100% | 75-100% | 0-25% | 0-25% | 75-100% |
| 821 | 75-100% | 75-100% | 75-100% | 75-100% | 25-75% | 75-100% |
| 822 | 75-100% | 75-100% | 75-100% | 75-100% | 25-75% | 75-100% |
| 823 | 75-100% | 75-100% | 75-100% | 25-75% | 25-75% | 75-100% |
| 824 | 75-100% | 75-100% | 75-100% | 25-75% | 25-75% | 75-100% |

ND = not determined

TABLE 8A
Sequences for the differentiation and/or detection of T-lymphocytes.

| SEQ ID NO: | |
|---|---|
| Genomic | Tissue |
| 511 | CD4 T-lymphocyte |
| 634 | CD4 T-lymphocyte |
| 637 | CD4 T-lymphocyte |
| 652 | CD4 T-lymphocyte |
| 416 | CD4 T-lymphocyte, CD8 T- |
| | lymphocyte |
| 417 | CD4 T-lymphocyte, CD8 T- |
| | lymphocyte |
| 426 | CD4 T-lymphocyte, CD8 T- |
| | lymphocyte |
| 438 | CD4 T-lymphocyte, CD8 T- |
| | lymphocyte |
| 442 | CD4 T-lymphocyte, CD8 T- |
| | lymphocyte |
| 445 | CD4 T-lymphocyte, CD8 T- |
| | lymphocyte |
| 449 | CD4 T-lymphocyte, CD8 T- |
| | lymphocyte |
| 451 | CD4 T-lymphocyte, CD8 T- |
| | lymphocyte |
| 452 | CD4 T-lymphocyte, CD8 T- |
| | lymphocyte |
| 458 | CD4 T-lymphocyte, CD8 T- |
| | lymphocyte |
| 467 | CD4 T-lymphocyte, CD8 T- |
| | lymphocyte |
| 468 | CD4 T-lymphocyte, CD8 T- |
| | lymphocyte |
| 474 | CD4 T-lymphocyte, CD8 T- |
| | lymphocyte |
| 481 | CD4 T-lymphocyte, CD8 T- |
| | lymphocyte |
| 482 | CD4 T-lymphocyte, CD8 T- |
| | lymphocyte |
| 483 | CD4 T-lymphocyte, CD8 T- |
| | lymphocyte |
| 489 | CD4 T-lymphocyte, CD8 T- |
| | lymphocyte |
| 490 | CD4 T-lymphocyte, CD8 T- |
| | lymphocyte |
| 491 | CD4 T-lymphocyte, CD8 T- |
| | lymphocyte |
| 492 | CD4 T-lymphocyte, CD8 T- |
| | lymphocyte |
| 493 | CD4 T-lymphocyte, CD8 T- |
| | lymphocyte |
| 497 | CD4 T-lymphocyte, CD8 T- |
| | lymphocyte |
| 513 | CD4 T-lymphocyte, CD8 T- |
| | lymphocyte |
| 515 | CD4 T-lymphocyte, CD8 T- |
| | lymphocyte |
| 518 | CD4 T-lymphocyte, CD8 T- |
| | lymphocyte |
| 522 | CD4 T-lymphocyte, CD8 T- |
| | lymphocyte |
| 527 | CD4 T-lymphocyte, CD8 T- |
| | lymphocyte |
| 528 | CD4 T-lymphocyte, CD8 T- |
| | lymphocyte |
| 531 | CD4 T-lymphocyte, CD8 T- |
| | lymphocyte |
| 535 | CD4 T-lymphocyte, CD8 T- |
| | lymphocyte |
| 542 | CD4 T-lymphocyte, CD8 T- |
| | lymphocyte |
| 554 | CD4 T-lymphocyte, CD8 T- |
| | lymphocyte |
| 555 | CD4 T-lymphocyte, CD8 T- |
| | lymphocyte |
| 557 | CD4 T-lymphocyte, CD8 T- |
| | lymphocyte |
| 558 | CD4 T-lymphocyte, CD8 T- |
| | lymphocyte |
| 562 | CD4 T-lymphocyte, CD8 T- |
| | lymphocyte |
| 566 | CD4 T-lymphocyte, CD8 T- |
| | lymphocyte |
| 574 | CD4 T-lymphocyte, CD8 T- |
| | lymphocyte |
| 588 | CD4 T-lymphocyte, CD8 T- |
| | lymphocyte |
| 597 | CD4 T-lymphocyte, CD8 T- |
| | lymphocyte |
| 598 | CD4 T-lymphocyte, CD8 T- |
| | lymphocyte |
| 607 | CD4 T-lymphocyte, CD8 T- |
| | lymphocyte |
| 632 | CD4 T-lymphocyte, CD8 T- |
| | lymphocyte |
| 639 | CD4 T-lymphocyte, CD8 T- |
| | lymphocyte |
| 651 | CD4 T-lymphocyte, CD8 T- |
| | lymphocyte |
| 659 | CD4 T-lymphocyte, CD8 T- |
| | lymphocyte |
| 669 | CD4 T-lymphocyte, CD8 T- |
| | lymphocyte |
| 675 | CD4 T-lymphocyte, CD8 T- |
| | lymphocyte |
| 676 | CD4 T-lymphocyte, CD8 T- |
| | lymphocyte |
| 682 | CD4 T-lymphocyte, CD8 T- |
| | lymphocyte |
| 683 | CD4 T-lymphocyte, CD8 T- |
| | lymphocyte |
| 685 | CD4 T-lymphocyte, CD8 T- |
| | lymphocyte |
| 691 | CD4 T-lymphocyte, CD8 T- |
| | lymphocyte |
| 695 | CD4 T-lymphocyte, CD8 T- |
| | lymphocyte |
| 697 | CD4 T-lymphocyte, CD8 T- |
| | lymphocyte |
| 710 | CD4 T-lymphocyte, CD8 T- |
| | lymphocyte |
| 463 | CD4 T-lymphocyte, CD8 T- |
| | lymphocyte |
| 465 | CD4 T-lymphocyte, CD8 T- |
| | lymphocyte |
| 679 | CD4 T-lymphocyte, CD8 T- |
| | lymphocyte |
| 480 | CD4 T-lymphocyte, CD8 T- |
| | lymphocyte |
| 701 | CD4 T-lymphocyte, CD8 T- |
| | lymphocyte |
| 706 | CD8 T-lymphocyte |

TABLE 8B
Sequences for the differentiation and/or detection of embryonic liver.

| SEQ ID NO: | |
|---|---|
| Genomic | Tissue |
| 509 | embryonic |
| | liver |
| 459 | embryonic |
| | liver |
| 475 | embryonic |
| | liver |
| 476 | embryonic |
| | liver |
| 477 | embryonic |
| | liver |
| 479 | embryonic |
| | liver |
| 484 | embryonic |
| | liver |
| 485 | embryonic |
| | liver |
| 552 | embryonic |
| | liver |
| 593 | embryonic |
| | liver |
| 596 | embryonic |
| | liver |
| 654 | embryonic |
| | liver |
| 712 | embryonic |
| | liver |
| 429 | embryonic |
| | liver |
| 465 | embryonic |
| | liver |
| 679 | embryonic |
| | liver |
| 480 | embryonic |
| | liver |
| 420 | embryonic |
| | liver |
| 612 | embryonic |
| | liver |
| 621 | embryonic |
| | liver |
| 624 | embryonic |
| | liver |
| 678 | embryonic |
| | liver |
| 505 | embryonic |
| | liver |

TABLE 8C
Sequences for the differentiation and/or detection of embryonic skeletal
muscle.

| SEQ ID NO: | |
|---|---|
| Genomic | Tissue |
| 444 | embryonic skeletal |
| | muscle |
| 658 | embryonic skeletal |
| | muscle |
| 441 | embryonic skeletal |
| | muscle |
| 459 | embryonic skeletal |
| | muscle |
| 475 | embryonic skeletal |
| | muscle |
| 476 | embryonic skeletal |
| | muscle |
| 477 | embryonic skeletal |
| | muscle |
| 479 | embryonic skeletal |
| | muscle |
| 484 | embryonic skeletal |
| | muscle |
| 485 | embryonic skeletal |
| | muscle |
| 552 | embryonic skeletal |
| | muscle |
| 593 | embryonic skeletal |
| | muscle |
| 596 | embryonic skeletal |
| | muscle |
| 654 | embryonic skeletal |
| | muscle |
| 712 | embryonic skeletal |
| | muscle |
| 429 | embryonic skeletal |
| | muscle |
| 465 | embryonic skeletal |
| | muscle |
| 679 | embryonic skeletal |
| | muscle |
| 480 | embryonic skeletal |
| | muscle |
| 420 | embryonic skeletal |
| | muscle |
| 612 | embryonic skeletal |
| | muscle |
| 621 | embryonic skeletal |
| | muscle |
| 624 | embryonic skeletal |
| | muscle |
| 678 | embryonic skeletal |
| | muscle |

TABLE 8D
Sequences for the differentiation and/or detection of fibroblasts:

| SEQ ID NO: | |
|---|---|
| Genomic | Tissue |
| 422 | fibroblast |
| 440 | fibroblast |
| 446 | fibroblast |
| 473 | fibroblast |
| 504 | fibroblast |
| 524 | fibroblast |
| 534 | fibroblast |
| 563 | fibroblast |
| 567 | fibroblast |
| 569 | fibroblast |
| 580 | fibroblast |
| 584 | fibroblast |
| 586 | fibroblast |
| 594 | fibroblast |
| 601 | fibroblast |
| 610 | fibroblast |
| 611 | fibroblast |
| 617 | fibroblast |
| 626 | fibroblast |
| 630 | fibroblast |
| 631 | fibroblast |
| 636 | fibroblast |
| 638 | fibroblast |
| 641 | fibroblast |
| 642 | fibroblast |
| 643 | fibroblast |
| 645 | fibroblast |
| 646 | fibroblast |
| 647 | fibroblast |
| 648 | fibroblast |
| 653 | fibroblast |
| 655 | fibroblast |
| 660 | fibroblast |
| 688 | fibroblast |
| 690 | fibroblast |
| 699 | fibroblast |
| 708 | fibroblast |
| 709 | fibroblast |
| 471 | fibroblast |
| 572 | fibroblast |
| 640 | fibroblast |
| 576 | fibroblast |

TABLE 8E
Sequences for the differentiation and/or detection of heart muscle.

| SEQ ID NO: | |
|---|---|
| Genomic | Tissue |
| 430 | heart muscle |
| 579 | heart muscle |
| 595 | heart muscle |
| 615 | heart muscle |
| 619 | heart muscle |
| 698 | heart muscle |
| 700 | heart muscle |
| 424 | heart muscle |

TABLE 8F
Sequences for the differentiation of heart muscle from skeletal muscle:

| SEQ ID NO: | |
|---|---|
| Genomic | Tissue |
| 525 | heart muscle versus skeletal muscle |
| 590 | heart muscle versus skeletal muscle |
| 599 | heart muscle versus skeletal muscle |
| 704 | heart muscle versus skeletal muscle |

TABLE 8G
Sequences for the differentiation and/or detection of keratinocytes.

| SEQ ID NO: | |
|---|---|
| Genomic | Tissue |
| 463 | keratinocyte |
| 433 | keratinocyte |
| 435 | keratinocyte |
| 439 | keratinocyte |
| 447 | keratinocyte |
| 450 | keratinocyte |
| 454 | keratinocyte |
| 460 | keratinocyte |
| 461 | keratinocyte |
| 462 | keratinocyte |
| 494 | keratinocyte |
| 495 | keratinocyte |
| 499 | keratinocyte |
| 501 | keratinocyte |
| 506 | keratinocyte |
| 512 | keratinocyte |
| 516 | keratinocyte |
| 520 | keratinocyte |
| 521 | keratinocyte |
| 526 | keratinocyte |
| 532 | keratinocyte |
| 533 | keratinocyte |
| 536 | keratinocyte |
| 539 | keratinocyte |
| 541 | keratinocyte |
| 549 | keratinocyte |
| 550 | keratinocyte |
| 551 | keratinocyte |
| 556 | keratinocyte |
| 559 | keratinocyte |
| 561 | keratinocyte |
| 564 | keratinocyte |
| 565 | keratinocyte |
| 570 | keratinocyte |
| 571 | keratinocyte |
| 575 | keratinocyte |
| 587 | keratinocyte |
| 589 | keratinocyte |
| 592 | keratinocyte |
| 600 | keratinocyte |
| 602 | keratinocyte |
| 603 | keratinocyte |
| 604 | keratinocyte |
| 605 | keratinocyte |
| 608 | keratinocyte |
| 609 | keratinocyte |
| 614 | keratinocyte |
| 625 | keratinocyte |
| 627 | keratinocyte |
| 628 | keratinocyte |
| 633 | keratinocyte |
| 635 | keratinocyte |
| 649 | keratinocyte |
| 650 | keratinocyte |
| 662 | keratinocyte |
| 663 | keratinocyte |
| 666 | keratinocyte |
| 667 | keratinocyte |
| 670 | keratinocyte |
| 677 | keratinocyte |
| 687 | keratinocyte |
| 689 | keratinocyte |
| 692 | keratinocyte |
| 696 | keratinocyte |
| 702 | keratinocyte |
| 703 | keratinocyte |
| 707 | keratinocyte |
| 709 | keratinocyte |
| 471 | keratinocyte |
| 572 | keratinocyte |
| 640 | keratinocyte |
| 576 | keratinocyte |
| 613 | keratinocyte |

TABLE 8H
Sequences for the differentiation and/or detection of liver.

| SEQ | |
|---|---|
| ID NO: Genomic | Tissue |
| 415 | liver |
| 427 | liver |
| 434 | liver |
| 448 | liver |
| 456 | liver |
| 457 | liver |
| 503 | liver |
| 508 | liver |
| 529 | liver |
| 537 | liver |
| 540 | liver |
| 546 | liver |
| 548 | liver |
| 553 | liver |
| 568 | liver |
| 582 | liver |
| 583 | liver |
| 591 | liver |
| 606 | liver |
| 616 | liver |
| 620 | liver |
| 661 | liver |
| 680 | liver |
| 684 | liver |
| 693 | liver |
| 705 | liver |
| 711 | liver |
| 701 | liver |
| 613 | liver |
| 505 | liver |

TABLE 8I
Sequences for the differentiation and/or detection of melanocytes.

| SEQ ID NO: | |
|---|---|
| Genomic | Tissue |
| 418 | melanocyte |
| 423 | melanocyte |
| 443 | melanocyte |
| 455 | melanocyte |
| 478 | melanocyte |
| 487 | melanocyte |
| 488 | melanocyte |
| 496 | melanocyte |
| 523 | melanocyte |
| 545 | melanocyte |
| 618 | melanocyte |
| 644 | melanocyte |
| 656 | melanocyte |
| 671 | melanocyte |
| 672 | melanocyte |
| 674 | melanocyte |
| 681 | melanocyte |
| 471 | melanocyte |
| 572 | melanocyte |
| 640 | melanocyte |
| 576 | melanocyte |

TABLE 8J
Sequences for the differentiation and/or detection of placenta.

| SEQ ID NO: | |
|---|---|
| Genomic | Tissue |
| 436 | placenta |
| 538 | placenta |
| 419 | placenta |

TABLE 8K
Sequences for the differentiation and/or detection of skeletal muscle.

| SEQ ID NO: | |
|---|---|
| Genomic | Tissue |
| 432 | skeletal muscle |
| 453 | skeletal muscle |
| 466 | skeletal muscle |
| 469 | skeletal muscle |
| 472 | skeletal muscle |
| 498 | skeletal muscle |
| 500 | skeletal muscle |
| 514 | skeletal muscle |
| 517 | skeletal muscle |
| 530 | skeletal muscle |
| 544 | skeletal muscle |
| 547 | skeletal muscle |
| 573 | skeletal muscle |
| 577 | skeletal muscle |
| 585 | skeletal muscle |
| 622 | skeletal muscle |
| 657 | skeletal muscle |
| 664 | skeletal muscle |
| 668 | skeletal muscle |
| 673 | skeletal muscle |
| 686 | skeletal muscle |
| 441 | skeletal muscle |
| 424 | skeletal muscle |
| 502 | skeletal muscle |
| 519 | skeletal muscle |
| 629 | skeletal muscle |
| 420 | skeletal muscle |
| 612 | skeletal muscle |
| 621 | skeletal muscle |
| 624 | skeletal muscle |
| 678 | skeletal muscle |
| 658 | skeletal muscle |

TABLE 8L
Sequences for the differentiation and/or detection of sperm.

| SEQ ID NO: | |
|---|---|
| Genomic | Tissue |
| 413 | sperm |
| 414 | sperm |
| 421 | sperm |
| 428 | sperm |
| 431 | sperm |
| 437 | sperm |
| 464 | sperm |
| 470 | sperm |
| 486 | sperm |
| 507 | sperm |
| 510 | sperm |
| 543 | sperm |
| 560 | sperm |
| 578 | sperm |
| 581 | sperm |
| 623 | sperm |
| 665 | sperm |
| 694 | sperm |

TABLE 9A
Sequences for the differentiation and/or detection of T-lymphocytes.

| SEQ ID NO: | |
|---|---|
| Genomic | Tissue |
| 730 | CD4 T-lymphocyte |
| 760 | CD4 T-lymphocyte, CD8 T- |
| | lymphocyte |
| 766 | CD4 T-lymphocyte, CD8 T- |
| | lymphocyte |
| 778 | CD4 T-lymphocyte, CD8 T- |
| | lymphocyte |
| 786 | CD4 T-lymphocyte, CD8 T- |
| | lymphocyte |
| 715 | CD4 T-lymphocyte, CD8 T- |
| | lymphocyte |
| 799 | CD4 T-lymphocyte, CD8 T- |
| | lymphocyte |

TABLE 9B
Sequences for the differentiation and/or detection of embryonic liver.

| SEQ ID NO: | |
|---|---|
| Genomic | Tissue |
| 752 | embryonic liver |
| 763 | embryonic liver |

TABLE 9C
Sequences for the differentiation and/or detection
of embryonic skeletal muscle.

| SEQ ID NO: | |
|---|---|
| Genomic | Tissue |
| 781 | embryonic skeletal |
| | muscle |
| 717 | embryonic skeletal |
| | muscle |
| 719 | embryonic skeletal |
| | muscle |
| 714 | embryonic skeletal |
| | muscle |

TABLE 9D
Sequences for the differentiation and/or detection of fibroblasts.

| SEQ ID NO: | |
|---|---|
| Genomic | Tissue |
| 720 | fibroblast |
| 723 | fibroblast |
| 725 | fibroblast |
| 733 | fibroblast |
| 737 | fibroblast |
| 738 | fibroblast |
| 753 | fibroblast |
| 755 | fibroblast |
| 758 | fibroblast |
| 761 | fibroblast |
| 764 | fibroblast |
| 767 | fibroblast |
| 768 | fibroblast |
| 769 | fibroblast |
| 770 | fibroblast |
| 774 | fibroblast |
| 780 | fibroblast |
| 782 | fibroblast |
| 784 | fibroblast |
| 788 | fibroblast |
| 792 | fibroblast |
| 801 | fibroblast |
| 809 | fibroblast |
| 814 | fibroblast |
| 817 | fibroblast |
| 818 | fibroblast |
| 718 | fibroblast |
| 741 | fibroblast |
| 751 | fibroblast |
| 773 | fibroblast |
| 823 | fibroblast |
| 772 | fibroblast |
| 800 | fibroblast |
| 821 | fibroblast |
| 744 | fibroblast |
| 717 | fibroblast |
| 719 | fibroblast |
| 714 | fibroblast |

TABLE 9E
Sequences for the differentiation and/or detection of heart muscle.

| SEQ ID NO: | |
|---|---|
| Genomic | Tissue |
| 759 | heart muscle |
| 822 | heart muscle |
| 824 | heart muscle |

TABLE 9F
Sequences for the differentiation and/or detection of keratinocytes.

| SEQ ID NO: | |
|---|---|
| Genomic | Tissue |
| 716 | keratinocyte |
| 724 | keratinocyte |
| 727 | keratinocyte |
| 728 | keratinocyte |
| 729 | keratinocyte |
| 732 | keratinocyte |
| 734 | keratinocyte |
| 739 | keratinocyte |
| 740 | keratinocyte |
| 743 | keratinocyte |
| 745 | keratinocyte |
| 748 | keratinocyte |
| 762 | keratinocyte |
| 765 | keratinocyte |
| 771 | keratinocyte |
| 776 | keratinocyte |
| 777 | keratinocyte |
| 785 | keratinocyte |
| 789 | keratinocyte |
| 791 | keratinocyte |
| 795 | keratinocyte |
| 796 | keratinocyte |
| 803 | keratinocyte |
| 804 | keratinocyte |
| 805 | keratinocyte |
| 806 | keratinocyte |
| 811 | keratinocyte |
| 812 | keratinocyte |
| 813 | keratinocyte |
| 815 | keratinocyte |
| 816 | keratinocyte |
| 819 | keratinocyte |
| 820 | keratinocyte |
| 778 | keratinocyte |
| 786 | keratinocyte |
| 741 | keratinocyte |
| 751 | keratinocyte |
| 773 | keratinocyte |
| 744 | keratinocyte |
| 717 | keratinocyte |
| 719 | keratinocyte |
| 714 | keratinocyte |
| 807 | keratinocyte |

TABLE 9G
Sequences for the differentiation and/or detection of liver.

| SEQ ID NO: | |
|---|---|
| Genomic | Tissue |
| 746 | liver |
| 757 | liver |
| 793 | liver |
| 763 | liver |
| 713 | liver |
| 787 | liver |
| 790 | liver |
| 715 | liver |
| 799 | liver |

TABLE 9H
Sequences for the differentiation and/or detection of melanocytes.

| SEQ ID NO: | |
|---|---|
| Genomic | Tissue |
| 721 | melanocyte |
| 722 | melanocyte |
| 726 | melanocyte |
| 731 | melanocyte |
| 735 | melanocyte |
| 736 | melanocyte |
| 742 | melanocyte |
| 747 | melanocyte |
| 749 | melanocyte |
| 750 | melanocyte |
| 754 | melanocyte |
| 756 | melanocyte |
| 775 | melanocyte |
| 779 | melanocyte |
| 794 | melanocyte |
| 808 | melanocyte |
| 810 | melanocyte |
| 744 | melanocyte |
| 717 | melanocyte |
| 719 | melanocyte |
| 714 | melanocyte |
| 718 | melanocyte |
| 741 | melanocyte |
| 751 | melanocyte |
| 773 | melanocyte |

TABLE 9I
Sequences for the differentiation and/or detection of placenta.

| SEQ ID NO: | |
|---|---|
| Genomic | Tissue |
| 823 | placenta |
| 824 | placenta |
| 820 | placenta |

TABLE 9J
Sequences for the differentiation and/or detection of skeletal muscle.

| SEQ ID NO: | |
|---|---|
| Genomic | Tissue |
| 783 | skeletal muscle |
| 797 | skeletal muscle |
| 798 | skeletal muscle |
| 802 | skeletal muscle |
| 752 | skeletal muscle |
| 823 | skeletal muscle |
| 772 | skeletal muscle |
| 800 | skeletal muscle |
| 821 | skeletal muscle |
| 759 | skeletal muscle |
| 822 | skeletal muscle |
| 824 | skeletal muscle |
| 820 | skeletal muscle |
| 713 | skeletal muscle |
| 787 | skeletal muscle |
| 790 | skeletal muscle |
| 807 | skeletal muscle |

### EXAMPLES

### Example 1

More than 300 specific regions of differential methylation have been identified in primary cells and tissues. Some of them are located in known promoter regions, while others are located in intra- or intergenic regions throughout the chromosomes 20, 22 and 6.

### a) Materials and Methods

Samples: Studied primary cell cultures included lymphocytes (selected and sorted by CD4 and CD8 antigen expression), melanocytes, keratinocytes and fibroblasts. Cells were harvested and kept at −80° C. until RNA isolation. Isolated RNA samples from Heart, Liver and Skeletal Muscle were purchased from commercial suppliers (Ambion) and kept at −80° C. until use in reverse transcription.

### Analyzed Genes

10 genes were selected for the expression analysis considering the location of regions of differential methylation. In all of them, the region of differential methylation located in the promoter region, the table below under results provides the names of the analyzed genes.

### Total RNA Isolation and RT-PCR:

Total RNA was isolated from cells and tissues using RNeasy kits (Qiagen, Hilden, Germany). Concentration and purity of the obtained RNA was determined spectrophotometrically, while the integrity was determined by electrophoresis on denaturing IM urea—2% agarose gel. cDNA was prepared using Omniscript RT kit (Qiagen, Hilden, Germany) with random hexamers in accordance to manufacturer's conditions. PCR was performed using 3 μl of the prepared cDNA, specific primers and HotStartTaq DNA polymerase kit in accordance with manufacturer's conditions. The PCR parameters used were: 15 min at 95° C. followed by 40 cycles of 1 min at 94° C., 1 min at the annealing temperature specific for each primer pair, and 1 min at 72° C., ending with a final extension for 10 min at 72° C. Primers were designed to bind in successive exons in order to avoid amplification in case contaminating genomic DNA was present. Amplification conditions for each fragment were determined experimentally by amplifying cDNA produced from Universal Human RNA (BioCat, Heidelberg, Germany), which is a pool total RNAs isolated from several tissues. The sequences of the specific primers for each analyzed gene are provided in the accompanying sequence listing according to the table below. It further shows the particular annealing temperature used in the amplification reaction. PCR products were separated through a 2.5% Agarose gel.

### b) Results

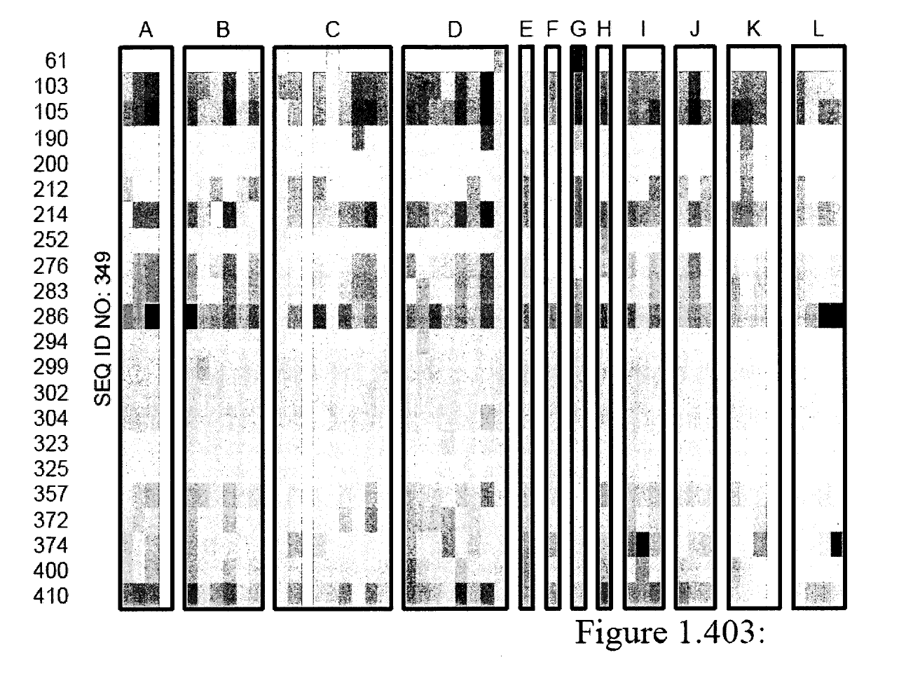

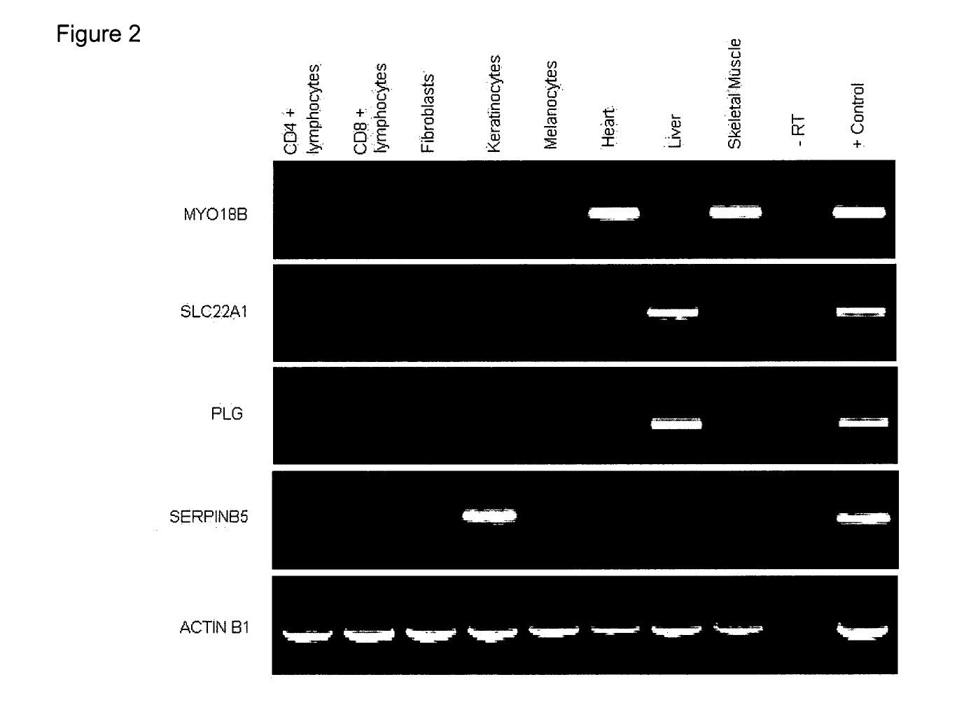

All analyzed genes showed a correlation between high methylation and gene silencing. In all of them, the region of differential methylation was located in the promoter region. Table 10 shows the results for each analyzed gene. As an example the results for the three genes MYO18B, SLC22A1 and PLG are shown in FIG. 2. As a control the expression of SERPINB5 was also studied. Finally the expression of a housekeeping gene in all the studied samples demonstrated the feasibility of the assay and gave a baseline for the semiquantitative analysis of the expression.

TABLE 10
Analyzed genes, primers and annealling temperatures and results.

| | | Differential | | | | |
|---|---|---|---|---|---|---|
| | | methylation | | | Annealing | Correlation |
| Gene | Gene SEQ | region | Forward Primer | Reverse Primer | temperature | methylation/ |
| Name | ID NO: | position | SEQ ID NO: | SEQ ID NO: | (° C.) | expression |
| RAET1E | 127 | Promoter | 4945 | 4946 | 60 | Inverse |
| PIB5PA | 53 | Promoter | 4947 | 4948 | 60 | Inverse |
| PLG | 171 | Promoter | 4949 | 4950 | 60 | Inverse |
| TGM3 | 180 | Promoter | 4951 | 4952 | 60 | Inverse |
| SLC22A1 | 170 | Promoter | 4953 | 4954 | 60 | Inverse |
| RP3- | 151 | Promoter | 4955 | 4956 | 60 | Inverse |
| 398D13.4 | | | | | | |
| OSM | 39 | Promoter | 4957 | 4958 | 63 | Inverse |
| MYO18B | 12 | Promoter | 4959 | 4960 | 60 | Inverse |
| CTA- | 43 | Promoter | 4961 | 4962 | 60 | Inverse |
| 299D3.6 | | | | | | |
| PARVG | 85 | Promoter | 4963 | 4964 | 60 | Inverse |
| SERPINB5 | | Promoter | 4965 | 4966 | 60 | Inverse |
| | | | | | | (Positive |
| | | | | | | control) |
| ACTB1 | | | 4967 | 4968 | 56 | No correlation |
| | | | | | | (Negative |
| | | | | | | control) |

### Example 2

### Large-Scale DNA Methylation Profiling of Human Chromosomes 6, 20 and 22

Using bisulfite DNA sequencing, we report high-resolution methylation reference profiles of human chromosomes 6, 20 and 22, providing a resource of about 1.9 million CpG measurements in 43 samples derived from 12 different (healthy) tissues.

It was the aim of the study to establish DNA methylation reference profiles for three human chromosomes from a number of healthy (no known disease phenotype) human tissues and primary cells. The study was further controlled for age and sex and comprised the analysis of 43 different samples derived from sperm, various primary cell types (dermal fibroblasts, dermal keratinocytes, dermal melanocytes, CD4⁺ and CD8⁺ lymphocytes) and tissues (heart muscle, skeletal muscle, liver and placenta, table 9). Tissues were pooled from up to three age- and sex-matched individuals (see table 9 for details). Primary cells were cultured for no more than three passages to minimize the risk of introducing aberrant methylation. Additionally, the methylation of selected amplicons were compared before and after cultivation with no difference in average methylation being detected. As dermal fibroblasts, keratinocytes and melanocytes are the major cell types constituting the human epidermis, we compared the average methylation of selected amplicons in these cell types with the corresponding values derived from additional human skin samples. No significant deviation between the methylation of the primary cells and tissues were detected, indicating that cell culturing for a limited number of passages does not change DNA methylation.

In total, we analyzed 2,524 amplicons associated with 873 genes on chromosomes 6, 20 and 22. Based on Ensembl (NCBI34) annotation, the amplicons were assigned to 6 distinct categories. Taking the number of biological and technical replicates into account, we have determined the methylation status of 1.88 million CpG sites. The corresponding data have been deposited into the public HEP database and can be accessed at www.epigenome.org.

### Material and Methods

Cell and Tissue samples: Tissue samples were obtained from following sources: Asterand, (Detroit, US), Pathlore Plc. (Nottingham, UK), Tissue Transformation Technologies (T-cubed, Edison, US), Northwest Andrology (Missoula, US), NDRI (Philadelphia, US) and Biocat GmBH (Heidelberg, Germany). Only anonymized samples were used and ethical approval was obtained for the study. Contamination by blood cells is estimated to be low as blood specific methylation profiles were not detected in the tissues. Human primary cells were acquired from Cascade Biologics (Mansfield, United Kingdom), Cell Applications Inc. (San Diego, United States), Analytical Biological Services Inc. (Wilmington, US), Cambrex Bio Science (Verviers, Belgium) and from the DIGZ (Berlin, Germany). Dermal fibroblasts, keratinocytes and melanocytes were cultured according to the supplier's recommendations up to a maximum of 3 passages reducing the risk of aberrant methylation due to extended culturing. CD4⁺ T-lymphocytes were isolated from fresh whole blood by depletion of CD4⁺ monocytes followed by a negative selection. CD8⁺ cells were isolated from fresh whole blood by positive selection. Subsequent FACS analysis confirmed a purity of CD4⁺/CD8⁺ T-lymphocytes greater than 90%. In some cases, DNA samples were pooled according to the sex and age of the donors. All genders were confirmed by sex-specific PCR.

Amplicon selection and classification: Amplicons were selected and classified based on Ensembl (build NCBI 34) annotation, amplicons were designed in the following genomic regions:

5′-UTR: Overlapping by at least 200 bp with or within a core region of 2,000 bp upstream to 500 bp downstream of the TSS. Where multiple sites were annotated per gene, the first annotated TSS was used.
Exonic: Greater than 50% and at least 200 bp of amplicon overlapping with annotated exon. Intronic: Greater than 50% and at least 200 bp of amplicon overlapping with annotated intron. ECR (evolutionary conserved regions): ≧70% DNA sequence similarity (including ≧4 CpGs) for at least 100 bp between human and mouse non-coding sequences. Out of 3,249 ECRs identified on chromosome 20, 290 intergenic and 206 intronic (496 in total) ECRs were selected.
Sp1: Overlapping with putative Sp1 sites identified by ChIP-chip analysis.
ncRNA: CD box snoRNAs as described by Lestrade, L. and Weber, M. J. snoRNA-LBME-db, a comprehensive database of human H/ACA and C/D box snoRNAs. Nucleic Acids Res. 34, 158-162 (2006) and miRNAs as reported by Griffiths-Jones, S. The microRNA Registry. Nucleic Acids Res. 32, 109-111 (2004) located on chromosome 22.
Other: amplicons that are not located within a gene or a 5′-UTR and additionally do not belong to any other category. CGI (CpG island) were classified based on the criteria by Gardiner-Garden and Frommer J. Mol. Biol. 196, 261-282 (1987).

DNA extraction, PCR amplification and sequencing: DNA was extracted using the Qiagen DNA Genomic-tip kit according the manufacturer's recommendation. After quantification, DNA was bisulfite converted as previously described in PCT/WO/2005/038051 (2005). Bisulfite-specific primers with a minimum length of 18 bp were designed using a modified primer-3 program. The target sequence of the designed primers contained no CpGs allowing for an unbiased amplification of both hypo- and hypermethylated DNAs. All primers were tested for their ability to yield high quality sequences. Primers that gave rise to an amplicon of the expected size using non-bisulfite treated DNA as a template were discarded, thus ensuring the specificity for bisulphite-converted DNAs. Primers were also tested for specificity on bisulfite DNA by electronic PCR. DNA amplification was set up in 96-well plates using an automated pipeline. PCR amplicons were quality controlled by agarose gel electrophoresis, re-arrayed into 384-well plates for high-throughput processing, cleaned up using ExoSAP-IT (USB Corporation, Cleveland, Ohio) to remove any excess nucleotides and primers and sequenced directly in the forward and reverse directions. Sequencing was performed on ABI 3730 capillary sequencers using 1/32nd dilution of ABI Prism BigDye terminator V3.1 sequencing chemistry after hotstart (96° C. for 30 seconds) thermocycling (92° C. for 5 seconds, 50° C. for 5 seconds, 60° C. for 120 seconds×44 cycles) and ethanol precipitation. PCR fragments were sequenced using the same PCR amplification primers. PCR primers for the differentially methylated amplificates are provided in Table 4. Trace files and methylation signals at a given CpG site were quantified using the software ESME as previously described in Bioinformatics 20, 3005-3012 (2004).

The bisulfite sequencing-based approach chosen here allows one to measure DNA methylation with high reproducibility and accuracy, as independent measurements are derived from both the sense and antisense strands of a PCR amplicon (R=0.87; N=557,837). In addition, about 4.1% of the amplicons were subjected to independent PCR amplification and sequencing. These technical replicates also displayed high correlation (R=0.9; N=15,655). Furthermore, the signal is independent of the position of the measured CpG within the amplicon, which is supported by high correlation between measurements of the same CpGs in overlapping amplicons (R=0.85; N=91,528).

RNA extraction and RT-PCR. Aliquots of the same samples of the human melanocytes, keratinocytes, fibroblasts, CD4⁺ and CD8⁺ cells that were used for methylation analysis were used for RNA analysis. Primary cell cultures (maximum of 3 passages) of human melanocytes, keratinocytes and dermal fibroblasts cells were harvested and kept at −80° C. until RNA isolation. Isolated RNA samples from heart, liver and skeletal muscle were purchased from Ambion (Austin, US) and kept at −80° C. until used for reverse transcription. Total RNA was isolated using the RNeasy kit from Qiagen (Hilden, Germany) followed by cDNA synthesis using the Omniscript RT kit from the same supplier and random hexamers. PCR (92° C. for 1 minute, 55-63° C. (depending on assay) for 1 minute, 72° C. for 1 minute for 30 to 40 cycles (depending on assay)) was performed using the HotStartTaq DNA polymerase kit (Qiagen) with 3 μl of the prepared cDNA and gene-specific primers. All kits were used according to the manufacturer's recommendations. PCR products were analyzed by electrophoresis on 2.5% agarose gels. Universal RNA was obtained from Biocat (Heidelberg, Germany) and total RNA isolated from brain and sperm from Stratagene (La Jolla, Calif., US).

Analysis and Statistical methods: Methylation profiles were calculated as described previously in PLoS Biol. 2, 2170-2182 (2004) and are available from the HEP database/browser at www.epigenome.org. The methylation profile of each individual amplicon is provided in FIGS. 1.1 to 1.403.

### Results

Each individual matrix represents the sequencing data for an individual amplificate. Each of the discrete blocks of the matrix represent a single sample type and are labeled ‘A’ through ‘L’, said letters representing in each case the following tissue/cell types: A: Melanocytes; B: Heart Muscle; C: Skeletal muscle; D: Liver; E: Sperm; F: Embryonic skeletal muscle; G: Embryonic liver; H: Placental; I: Fibroblast; J: Keratinocytes; K: CD8; L: CD4.

The SEQ ID NO: of the genomic region of each amplificate is shown to the left of the matrices. This may be cross referenced in Table 4 to determine the amplificate and primer sequences. Each row of a matrix represents a single CpG site within the amplificate (according to the corresponding SEQ ID NO: from Table 4) and is numbered accordingly, each column represents a single pooled DNA sample.

The degree of methylation is represented by the shade of each position within the column from black representing 100% methylation to light gray representing 0% methylation. White positions represented a measurement for which no data was available.

### Differentially Methylated Regions (DMRS)

Kruskall-Wallis tests were used to determine differential methylation between tissues, measuring the proportion of uncorrected p-values that were smaller 0.001 for all CpGs. As this test is insensitive to samples that were only measured in a single sample such as sperm and placenta, the obtained number of DMRs is unlikely to be overstated due to putative aberrant methylation within these samples. Some DMRs were experimentally validated by sequencing independent DNA samples. Equality between two groups (age and sex) was performed using Wilcoxon tests.

Median CpG methylation values were used for the analysis of co-methylation. CpGs for which methylation values derived from both the forward and reverse strands displayed a difference of greater than 10% between the two values were excluded. Methylation changes were calculated based on the absolute methylation differences between CpG pairs of identical samples. To exclude a bias introduced by the amplicon selection, the analysis was performed using both, individual CpGs (window size 20,000 bp) and CpGs of the same amplicons. Co-methylation of CpGs was described as a function of the distance (in bp) displaying the observed ratio of similar methylation degree.

For scatter plots, equal amounts of measurements were binned and ranked by numerical order of the X-axis values, representing means of X− and Y− data. For box plots and histograms, data were binned according to the intervals indicated on the X-axis containing different numbers of measurements.

As a measure for the probability of differential methylation, amplicons were sorted by their p-value and binned by rank into groups of 200 and scanned using 211 vertebrate position-weight matrices from the TRANSFAC library40 (version 3.2). For each motif, we picked a threshold such that it matched around 40% of amplicons and performed a □2 test to determine if the hit rate of the motif varied significantly between the highest and lowest 200 amplicons, ranked by P-value of differential methylation.

Tables 6 and 7 provide an overview of the genomic regions and ranges of methylation within which the actual value of methylation of said genomic regions lies specific of the said cells, tissues and/or organs. The actual value of methylation is shown in FIGS. 1.1-1.403.

Table 11 provides an overview of 43 different samples derived from sperm, various primary cell types (dermal fibroblasts, dermal keratinocytes, dermal melanocytes, CD4⁺ and CD8⁺ lymphocytes) and tissues (heart muscle, skeletal muscle, liver and placenta. The study was controlled for age and sex and comprised the analysis. Tissues were pooled from up to three age- and sex-matched individuals.

Table 12 provides summary statistics.

Table 13 provides an overview of the most preferred of the genes or genomic regions and ranges of methylation within which the actual value of methylation of said genomic regions lies specific of the said cells, tissues and/or organs. The actual value of methylation is shown in FIGS. 1.1-1.403. We found 17% of the 873 analyzed genes differentially methylated in their 5′-untranslated regions (5′-UTR), in at least one of the tissues examined. Differential methylation is observed more frequently in evolutionary conserved regions (ECRs) than within 5′-UTRs, suggesting that methylation has a functional role beyond a direct effect on transcription via promoter methylation. About one third of the differentially methylated 5′-UTRs are inversely correlated with transcription of the respective mRNAs. We did not find any significant sex or age-dependent differences in our study, indicating that methylation is ontogenetically more stable than previously anticipated.

TABLE 11

| | | Age | | |
|---|---|---|---|---|
| # | Tissue/cell type | (average) | Sex | Pool of |
| 1 | heart muscle | 25 Y | female | 3 |
| 2 | heart muscle | 23 Y | male | 3 |
| 3 | heart muscle | 25 Y | male | 3 |
| 4 | heart muscle | 78 Y | male | 3 |
| 5 | heart muscle | 74 Y | male | 3 |
| 6 | heart muscle | 62 Y | male | 3 |
| 7 | skeletal muscle | 19 Y | male | 3 |
| 8 | skeletal muscle | 30 Y | male | 3 |
| 9 | skeletal muscle | 22 Y | male | 3 |
| 10 | skeletal muscle | 59 Y | male | 3 |
| 11 | skeletal muscle | 67 Y | male | 3 |
| 12 | skeletal muscle | 74 Y | male | 3 |
| 13 | skeletal muscle | 65 Y | female | 3 |
| 14 | skeletal muscle | 67 Y | female | 3 |
| 15 | skeletal muscle | 84 Y | female | 2 |
| 16 | liver | 23 Y | male | 3 |
| 17 | liver | 23 Y | male | 3 |
| 18 | liver | 30 Y | male | 3 |
| 19 | liver | 82 Y | male | 3 |
| 20 | liver | 65 Y | male | 3 |
| 21 | liver | 74 Y | male | 3 |
| 22 | liver | 70 Y | female | 3 |
| 23 | liver | 81 Y | female | 3 |
| 24 | sperm | 24 Y | male | 3 |
| 25 | fetal skeletal muscle | 23 W | male | 3 |
| 26 | fetal liver | 24 W | male | 3 |
| 27 | placenta | NB | female | 3 |
| 28 | melanocytes | 30 Y | female | 1 |
| 29 | melanocytes | 41 Y | female | 1 |
| 30 | melanocytes | 42 Y | female | 1 |
| 31 | dermal fibroblasts | 43 Y | female | 2 |
| 32 | dermal fibroblasts | 41 Y | female | 2 |
| 33 | dermal fibroblasts | 39 Y | female | 2 |
| 34 | dermal keratinocytes | 43 Y | female | 3 |
| 35 | dermal keratinocytes | 39 Y | female | 3 |
| 36 | dermal keratinocytes | 33 Y | female | 2 |
| 37 | CD4 lymphocytes | 20 Y | male | 1 |
| 38 | CD8 lymphocytes | 59 Y | male | 1 |
| 39 | CD8 lymphocytes | 60/61 Y | female | 1 |
| 40 | CD8 lymphocytes | 62 Y | male | 1 |
| 41 | CD4 lymphocytes | 59/60 Y | female | 1 |
| 42 | CD4 lymphocytes | 61 Y | female | 1 |
| 43 | CD4 lymphocytes | 62 Y | female | 1 |

Table 11 provides an overview of 43 different samples derived from sperm, various primary cell types (dermal fibroblasts, dermal keratinocytes, dermal melanocytes, CD4⁺ and CD8⁺ lymphocytes) and tissues (heart muscle, skeletal muscle, liver and placenta.

TABLE 12

| | | Chromosome | Chromosome | Chromosome |
|---|---|---|---|---|
| | Total | 6 | 20 | 22 |
| CpG islands on chromosome | 2,279 | 1,07 | 662 | 547 |
| CpG islands covered | 511 | 256 | 29 | 226 |
| CpG islands percentage covered | 22% | 24% | 4% | 41% |
| Genes covered | 873 | 383 | 89 | 401 |
| Exons covered | 853 | 454 | 23 | 376 |
| Introns covered | 920 | 465 | 118 | 337 |
| Number of tissues analyzed | 12 | | | |
| Number of samples analyzed | 43 | | | |
| Average length of amplicon +/− SD | 411 +/− | | | |
| | 77 bp | | | |
| Average number of CpGs per | 16 +/− 10.8 | | | |
| amplicon | | | | |
| Total number of different amplicons | 2,524 | | | |
| Number of CpGs analyzed | 1,885,003 | | | |

Table 12 provides summary statistics.

TABLE 13

| Gene | Ensembl ID | CD4 T-lymphocytes | CD8 T-lymphocytes | Embryonic liver | Em. ske. muscle |
|---|---|---|---|---|---|
| ZNRF3 | ENSG00000183579 | 90 | 90 | 80 | 30 |
| SLC7A4 | OTTHUMG00000030129 | 0 | 0 | 0 | 0 |
| Myosin-18B | ENSG00000133454 | 100 | 90 | 80 | 40 |
| (MYO18B) | | | | | |
| Glycoprotein Ib | OTTHUMT00000075045 | 10 | 10 | 10 | 70 |
| (platelet), beta | | | | | |
| polypeptide | | | | | |
| RP1-47A17.8 | OTTHUMG00000030878 | 80 | 80 | 70 | 20 |
| Oncostatin M | ENSG00000099985 | 0 | 0 | 40 | 90 |
| (OSM) | | | | | |
| CTA-299D3.6 | OTTHUMG00000030140 | 90 | 90 | 80 | 70 |
| CTA-941F9.6 | OTTHUMG00000030231 | 90 | 90 | 90 | 80 |
| Cytohesin-4 | ENSG00000100055 | 0 | 0 | 30 | 50 |
| PIB5PA | ENSG00000185133 | 60 | 70 | 60 | 10 |
| SUSD2 | ENSG00000099994 | 100 | 90 | 80 | 40 |
| No gene associated | NA | 90 | 90 | 80 | 30 |
| RP3-438O4.2 | OTTHUMG00000030922 | 90 | 90 | 90 | 30 |
| RP4-756G23.1 | OTTHUMG00000030852 | 90 | 90 | 80 | 50 |
| Somatostatin | ENSG00000183473 | 90 | 90 | 80 | 30 |
| receptor type 3 | | | | | |
| (SSTR3) | | | | | |
| Bcl-2 interacting | ENSG00000100290 | 20 | 40 | 40 | 50 |
| killer (BIK) | | | | | |
| GAS2L1 | ENSG00000185340 | 90 | 90 | 90 | 80 |
| RP3-355C18.2 | OTTHUMG00000030072 | 60 | 60 | 80 | 70 |
| Gamma-parvin | ENSG00000138964 | 30 | 20 | 70 | 90 |
| CARMA 3 | ENSG00000100065 | 100 | 100 | 90 | NA |
| TMPRSS6 | ENSG00000187045 | 10 | 30 | 50 | 0 |
| Platelet-derived | ENSG00000100311 | 20 | 20 | 20 | 0 |
| growth factor B | | | | | |
| chain precursor | | | | | |
| (PDGFB) | | | | | |
| CELSR1 Cadherin | ENSG00000075275 | 10 | 10 | 40 | 60 |
| T-box transcription | ENSG00000184058 | 10 | 10 | NA | 40 |
| factor (TBX1) | | | | | |
| Q6ZRW2_HUMAN | ENSG00000178199 | 40 | 30 | 70 | 30 |
| NKG2DL4 | ENSG00000164520 | 70 | 90 | 70 | 70 |
| (RAET1E) | | | | | |
| DAAM2 | ENSG00000146122 | 90 | 90 | 90 | 60 |
| RP1-47M23.1 | OTTHUMG00000015313 | 10 | 0 | 20 | 40 |
| RP11-397G17.1 | OTTHUMG00000014829 | 90 | 90 | 80 | 90 |
| Nesprin-1 (Syne-1) | ENSG00000131018 | 100 | 100 | 70 | NA |
| Myogenic repressor | ENSG00000112559 | 80 | 80 | 50 | 50 |
| I-mf (MDFI) | | | | | |
| RP11-174C7.4 | OTTHUMG00000015553 | 80 | 80 | 80 | 40 |
| CMAH | OTTHUMG00000016099 | 90 | 100 | 90 | 80 |
| PKHD1 | ENSG00000170927 | 90 | 80 | 70 | 50 |
| glutathione | OTTHUMG00000016307 | 20 | 10 | 30 | 80 |
| peroxidase 5 | | | | | |
| (GPX5) | | | | | |
| GRIK2 | ENSG00000164418 | 10 | 20 | 10 | 10 |
| SLC22A1 | ENSG00000112499 | 100 | 100 | 100 | 90 |
| RP11-235G24.1 | OTTHUMG00000015959 | 90 | 90 | 80 | 90 |
| TBX18 | ENSG00000112837 | 10 | 20 | 0 | 40 |
| TGM3 | ENSG00000125780 | 90 | 80 | 70 | NA |
| RIN2 | OTTHUMG00000031996 | 30 | 50 | 40 | 20 |
| SLC24A3 | OTTHUMG00000031993 | 100 | 100 | 100 | 60 |
| Q9ULE8_HUMAN | ENSG00000188559 | 100 | 100 | 80 | NA |
| C20orf117 | OTTHUMG00000032395 | 80 | 90 | 70 | 10 |
| Breast carcinoma | ENSG00000124243 | 10 | 10 | 10 | 20 |
| amplified sequence | | | | | |
| 4 (BCAS4) | | | | | |
| nuclear factor of | OTTHUMG00000032747 | 100 | 90 | 90 | 40 |
| activated T-cells | | | | | |
| (NFATC2) | | | | | |
| SCUBE1 | ENSG00000159307 | 10 | 10 | 0 | 40 |

| Gene | Fibroblasts | Heart muscle | Keratinocytes | Liver | Melanocytes | Placenta | Skelatal muscle | Sperm |
|---|---|---|---|---|---|---|---|---|
| ZNRF3 | 80 | 70 | 90 | 90 | 90 | 60 | 30 | 100 |
| SLC7A4 | 0 | 0 | 10 | 10 | 50 | 10 | 0 | 0 |
| Myosin-18B | 90 | 60 | 90 | 90 | 90 | 50 | 40 | NA |
| (MYO18B) | | | | | | | | |
| Glycoprotein Ib | 90 | 20 | 40 | 10 | 50 | 70 | 80 | 0 |
| (platelet), beta | | | | | | | | |
| polypeptide | | | | | | | | |
| RP1-47A17.8 | 20 | 80 | 90 | 80 | 70 | 70 | 60 | 90 |
| Oncostatin M | 90 | 90 | 100 | 90 | 100 | 90 | 90 | 90 |
| (OSM) | | | | | | | | |
| CTA-299D3.6 | 70 | 70 | 90 | 80 | 30 | 70 | 70 | 100 |
| CTA-941F9.6 | 90 | 90 | 10 | 80 | 90 | 90 | 80 | 90 |
| Cytohesin-4 | 50 | 50 | 10 | 50 | 60 | 30 | 50 | 90 |
| PIB5PA | 10 | 10 | 10 | 20 | 10 | 10 | 10 | 60 |
| SUSD2 | 50 | 80 | 40 | 60 | 50 | 60 | 50 | 90 |
| No gene associated | 10 | 40 | 10 | 60 | 60 | 20 | 20 | 90 |
| RP3-438O4.2 | 10 | 70 | 20 | 80 | 20 | 20 | 20 | 90 |
| RP4-756G23.1 | 50 | 80 | 80 | 90 | 20 | 70 | 50 | 90 |
| Somatostatin | 10 | 20 | 40 | 30 | 20 | 20 | 20 | 90 |
| receptor type 3 | | | | | | | | |
| (SSTR3) | | | | | | | | |
| Bcl-2 interacting | 80 | 80 | 90 | 80 | 80 | 80 | 50 | 90 |
| killer (BIK) | | | | | | | | |
| GAS2L1 | 80 | 80 | 10 | 90 | 90 | 90 | 70 | 20 |
| RP3-355C18.2 | 60 | 80 | 10 | 70 | 80 | 50 | 50 | 100 |
| Gamma-parvin | 90 | 90 | 90 | 80 | 90 | 80 | 90 | 100 |
| CARMA 3 | 80 | 90 | 80 | 100 | 90 | 80 | 50 | 100 |
| TMPRSS6 | 10 | 30 | 60 | 80 | 10 | 30 | 0 | 0 |
| Platelet-derived | 50 | 30 | 10 | 70 | 70 | 20 | 20 | 0 |
| growth factor B | | | | | | | | |
| chain precursor | | | | | | | | |
| (PDGFB) | | | | | | | | |
| CELSR1 Cadherin | 70 | 70 | 80 | 80 | 80 | 80 | 60 | 0 |
| T-box transcription | 80 | 10 | 0 | 20 | 70 | 80 | 70 | NA |
| factor (TBX1) | | | | | | | | |
| Q6ZRW2_HUMAN | 30 | 60 | 20 | 80 | 40 | 60 | 20 | 100 |
| NKG2DL4 | 50 | 80 | 0 | 100 | 70 | 50 | 50 | 100 |
| (RAET1E) | | | | | | | | |
| DAAM2 | 90 | 90 | 90 | 20 | 90 | 90 | 80 | 100 |
| RP1-47M23.1 | 40 | 80 | 70 | 40 | 60 | 60 | 60 | 90 |
| RP11-397G17.1 | 80 | 90 | 20 | 50 | 80 | 70 | 80 | 90 |
| Nesprin-1 (Syne-1) | 10 | 20 | 10 | 40 | 0 | 60 | 20 | 90 |
| Myogenic repressor | 80 | 40 | 0 | 50 | 60 | 50 | 80 | 0 |
| I-mf (MDFI) | | | | | | | | |
| RP11-174C7.4 | 0 | 50 | 70 | 80 | 50 | 40 | 30 | 80 |
| CMAH | 90 | 90 | 0 | 90 | 100 | 90 | 80 | 0 |
| PKHD1 | 40 | 50 | 10 | 40 | 30 | 50 | 60 | 100 |
| glutathione | 40 | 50 | 90 | 70 | 40 | 80 | 70 | 100 |
| peroxidase 5 | | | | | | | | |
| (GPX5) | | | | | | | | |
| GRIK2 | 80 | 20 | 90 | 10 | 70 | 50 | 10 | 10 |
| SLC22A1 | 100 | 100 | 100 | 60 | 100 | 90 | 100 | 100 |
| RP11-235G24.1 | 50 | 80 | 90 | 60 | 70 | 80 | 90 | 80 |
| TBX18 | 90 | 30 | 10 | 20 | 60 | 40 | 60 | 10 |
| TGM3 | 70 | 70 | 30 | 90 | 80 | 50 | 40 | 90 |
| RIN2 | 20 | 20 | 90 | 90 | 20 | 30 | 10 | NA |
| SLC24A3 | 90 | 80 | 90 | 90 | 90 | NA | 50 | NA |
| Q9ULE8_HUMAN | 90 | 100 | 0 | 60 | 100 | 90 | 100 | 100 |
| C20orf117 | 0 | 30 | 10 | 50 | 10 | 20 | 40 | 10 |
| Breast carcinoma | 20 | 20 | 10 | 20 | 70 | 20 | 30 | 80 |
| amplified sequence | | | | | | | | |
| 4 (BCAS4) | | | | | | | | |
| nuclear factor of | 10 | 70 | 10 | 50 | 10 | 40 | 40 | 100 |
| activated T-cells | | | | | | | | |
| (NFATC2) | | | | | | | | |
| SCUBE1 | 70 | 60 | 50 | 20 | 80 | 60 | 70 | 70 |

Table 13 provides an overview of the most preferred of the genes or genomic regions and ranges of methylation within which the actual value of methylation of said genomic regions lies specific of the said cells, tissues and/or organs. The actual value of methylation is shown in FIGS. 1.1-1.403.

### LITERATURE

Murakami M, Masuda S, Shimbara S, Ishikawa Y, Ishii T, Kudo I. Cellular distribution, post-translational modification, and tumorigenic potential of human group III secreted phospholipase A(2). J Biol Chem. 2005 Jul. 1; 280(26):24987-98. Epub 2005 Apr. 29.
Habib S J, Waizenegger T, Lech M, Neupert W, Rapaport D. Assembly of the TOB complex of mitochondria. J Biol Chem. 2005 Feb. 25; 280(8):6434-40. Epub 2004 Dec. 7.
Igarashi P, Shao X, McNally B T, Hiesberger T. Roles of HNF-1beta in kidney development and congenital cystic diseases. Kidney Int. 2005 November; 68(5):1944-7.
Bergmann C, Kupper F, Domia C, Schneider F, Senderek J, Zerres K. Algorithm for efficient PKHD1 mutation screening in autosomal recessive polycystic kidney disease (ARPKD). Hum Mutat. 2005 March; 25(3):225-31.
Menezes L F, Cai Y, Nagasawa Y, Silva A M, Watkins M L, Da Silva A M, Somlo S, Guay-Woodford L M, Germino G G, Onuchic L F. Polyductin, the PKHD1 gene product, comprises isoforms expressed in plasma membrane, primary cilium, and cytoplasm. Kidney Int. 2004 October; 66(4):1345-55.
Gresh L, Fischer E, Reimann A, Tanguy M, Garbay S, Shao X, Hiesberger T, Fiette L, Igarashi P, Yaniv M, Pontoglio M. A transcriptional network in polycystic kidney disease. EMBO J. 2004 Apr. 7; 23(7): 1657-68. Epub 2004 Mar. 18.
Zhang M Z, Mai W, Li C, Cho S Y, Hao C, Moeckel G, Zhao R, Kim I, Wang J, Xiong H, Wang H, Sato Y, Wu Y, Nakanuma Y, Lilova M, Pei Y, Harris R C, Li S, Coffey R J, Sun L, Wu D, Chen X Z, Breyer M D, Zhao Z J, McKanna J A, Wu G. PKHD1 protein encoded by the gene for autosomal recessive polycystic kidney disease associates with basal bodies and primary cilia in renal epithelial cells. Proc Natl Acad Sci USA. 2004 Feb. 24; 101(8):2311-6.
Onuchic L F, Furu L, Nagasawa Y, Hou X, Eggermann T, Ren Z, Bergmann C, Senderek J, Esquivel E, Zeltner R, Rudnik-Schoneborn S, Mrug M, Sweeney W, Avner E D, Zerres K, Guay-Woodford L M, Somlo S, Germino G G. PKHD1, the polycystic kidney and hepatic disease I gene, encodes a novel large protein containing multiple immunoglobulin-like plexin-transcription-factor domains and parallel beta-helix I repeats. Am J Hum Genet. 2002 May; 70(5):1305-17. Epub 2002 Mar. 15.
McCann J A, Xu Y Q, Frechette R, Guazzarotti L, Polychronakos C. The insulin-like growth factor-II receptor gene is associated with type I diabetes: evidence of a maternal effect. J Clin Endocrinol Metab. 2004 November; 89(11):5700-6. PMID: 15531531
Grundemann, D.; Gorboulev, V.; Gambaryan, S.; Veyhl, M.; Koepsell, H. Drug excretion mediated by a new prototype of polyspecific transporter. Nature 372: 549-552, 1994.
Leabman, M. K.; Huang, C. C.; DeYoung, J.; Carlson, E. J.; Taylor, T. R.; de la Cruz, M.; Johns, S. J.; Stryke, D.; Kawamoto, M.; Urban, T. J.; Kroetz, D. L.; Ferrin, T. E.: Clark, A. G.; Risch, N.; Herskowitz, I.; Giacomini, K. M.; {Pharmacogenetics of Membrane Transporters Investigators}: Natural variation in human membrane transporter genes reveals evolutionary and functional constraints. Proc. Nat. Acad. Sci. 100: 5896-5901, 2003.
Shu, Y.; Leabman, M. K.; Feng, B.; Mangravite, L. M.; Huang, C. C.; Stryke, D.; Kawamoto, M.; Johns, S. J.; DeYoung, J.; Carlson, E.; Ferrin, T. E.; Herskowitz, 1.; Giacomini, K. M.; Pharmacogenetics of Membrane Transporters Investigators: Evolutionary conservation predicts function of variants of the human organic cation transporter, OCT1. Proc. Nat. Acad. Sci. 100: 5902-5907, 2003.
Bourdet D L, Pritchard J B, Thakker D R. Differential substrate and inhibitory activities of ranitidine and famotidine toward human organic cation transporter 1 (hOCT1; SLC22A1), hOCT2 (SLC22A2), and hOCT3 (SLC22A3). J Pharmacol Exp Ther. 2005 December; 315(3):1288-97
Lips K S, Volk C, Schmitt B M, Pfeil U, Arndt P, Miska D, Ermert L, Kummer W, Koepsell H. Polyspecific cation transporters mediate luminal release of acetylcholine from bronchial epithelium. Am J Respir Cell Mol Biol. 2005 July; 33(1):79-88.)
Grover B, Buckley D, Buckley A R, Cacini W. Reduced expression of organic cation transporters ROCT1 and rOCT2 in experimental diabetes. J Pharmacol Exp Ther. 2004 March; 308(3):949-56.)
Wang D S, Kusuhara H, Kato Y, Jonker J W, Schinkel A H, Sugiyama Y. Involvement of organic cation transporter I in the lactic acidosis caused by metformin. Mol Pharmacol. 2003 April; 63(4):844-8.)
Sleutels F, Zwart R, Barlow D P. The non-coding Air RNA is required for silencing autosomal imprinted genes. Nature. 2002 Feb. 14; 415(6873):810-3.
Grover B, Buckley D, Buckley A R, Cacini W. Reduced expression of organic cation transporters rOCT1 and rOCT2 in experimental diabetes. J Pharmacol Exp Ther. 2004 March; 308(3):949-56. Epub 2004 Jan. 8.
Karbach U, Kricke J, Meyer-Wentrup F, Gorboulev V, Volk C, Loffing-Cueni D, Kaissling B, Bachmann S, Koepsell H. Localization of organic cation transporters OCT1 and OCT2 in rat kidney. Am J Physiol Renal Physiol. 2000 October; 279(4):F679-87.
Endoh M, Tamura G, Honda T, Homma N, Terashima M, Nishizuka S, Motoyama T. RASSF2, a potential tumor suppressor, is silenced by CpG island hypermethylation in gastric cancer. Br J Cancer. 2005 Dec. 12; 93(12):1395-1399.
Akino K, Toyota M, Suzuki H, Mita H, Sasaki Y, Ohe-Toyota M, Issa J P, Hinoda Y, Imai K, Tokino T. The Ras effector RASSF2 is a novel tumor-suppressor gene in human colorectal cancer. Gastroenterology. 2005 July; 129(1):156-69.
Hesson L B, Wilson R, Morton D, Adams C, Walker M, Maher E R, Latif F. CpG island promoter hypermethylation of a novel Ras-effector gene RASSF2A is an early event in colon carcinogenesis and correlates inversely with K-ras mutations. Oncogene. 2005 Jun. 2; 24(24):3987-94.
Vos M D, Ellis C A, Elam C, Ulku A S, Taylor B J, Clark G J. RASSF2 is a novel K-Ras-specific effector and potential tumor suppressor. J Biol Chem. 2003 Jul. 25; 278(30):28045-51. Epub 2003 May 5. PMID: 12732644
Ooms L M, Fedele C G, Astle M V, Ivetac I, Cheung V, Pearson R B, Layton M J, Forrai A, Nandurkar H H, Mitchell C A. The Inositol Polyphosphate 5-Phosphatase, PIPP, Is a Novel Regulator of Phosphoinositide 3-Kinase-dependent Neurite Elongation. Mol Biol Cell 2005 Nov. 9;
Ohkura S, Kondoh N, Hada A, Arai M, Yamazaki Y, Sindoh M, Takahashi M, Matsumoto I, Yamamoto M. Differential expression of the keratin-4, -13, -14, -17 and transglutaminase 3 genes during the development of oral squamous cell carcinoma from leukoplakia. Oral Oncol. 2005 July; 41(6):607-13. Epub 2005 Apr. 14.
Zhang J, Zhi H Y, Ding F, Luo A P, Liu Z H. Transglutaminase 3 expression in C57BL/6J mouse embryo epidermis and the correlation with its differentiation. Cell Res. 2005 February; 15(2): 105-10.
Zainelli G M, Dudek N L, Ross C A, Kim S Y, Muma N A. Mutant huntingtin protein: a substrate for transglutaminase 1, 2, and 3. J Neuropathol Exp Neurol. 2005 January; 64(1):58-65.
Raghunath M, Tontsidou L, Oji V, Aufenvenne K, Schurmeyer-Horst F, Jayakumar A, Stander H, Smolle J, Clayman G L, Traupe H. SPINK5 and Netherton syndrome: novel mutations, demonstration of missing LEKTI, and differential expression of transglutaminases. J Invest Dermatol. 2004 September; 123(3):474-83.
Luo A, Kong J, Hu G, Liew C C, Xiong M, Wang X, Ji J, Wang T, Zhi H, Wu M, Liu Z. Discovery of Ca2+-relevant and differentiation-associated genes downregulated in esophageal squamous cell carcinoma using cDNA microarray. Oncogene. 2004 Feb. 12; 23(6):1291-9.
Gonzalez H E, Gujrati M, Frederick M, Henderson Y, Arumugam J, Spring P W, Mitsudo K, Kim H W, Clayman G L. Identification of 9 genes differentially expressed in head and neck squamous cell carcinoma. Arch Otolaryngol Head Neck Surg. 2003 July; 129(7):754-9.
Hitomi K, Presland R B, Nakayama T, Fleckman P, Dale B A, Maki M. Analysis of epidermal-type transglutaminase (transglutaminase 3) in human stratified epithelia and cultured keratinocytes using monoclonal antibodies. J Dermatol Sci. 2003 August; 32(2):95-103.
Nakano T, Tani M, Nishioka M, Kohno T, Otsuka A, Ohwada S, Yokota J. Genetic and epigenetic alterations of the candidate tumor-suppressor gene MYO18B, on chromosome arm 22q, in colorectal cancer. Genes Chromosomes Cancer. 2005 June; 43(2):162-71.
Yanaihara N, Nishioka M, Kohno T, Otsuka A, Okamoto A, Ochial K, Tanaka T, Yokota J. Reduced expression of MYO18B, a candidate tumor-suppressor gene on chromosome arm 22q, in ovarian cancer. Int J Cancer. 2004 Oct. 20; 112(1):150-4.
Nishioka M, Kohno T, Tani M, Yanaihara N, Tomizawa Y, Otsuka A, Sasaki S, Kobayashi K, Niki T, Maeshima A, Sekido Y, Minna J D, Sone S, Yokota J. MYO18B, a candidate tumor suppressor gene at chromosome 22q12.1, deleted, mutated, and methylated in human lung cancer. Proc Natl Acad Sci USA. 2002 Sep. 17; 99(19):12269-74. Epub 2002 Sep. 3.
Kumar U, Grigorakis S I, Watt H L, Sasi R, Snell L, Watson P, Chaudhari S. Somatostatin receptors in primary human breast cancer: quantitative analysis of mRNA for subtypes 1-5 and correlation with receptor protein expression and tumor pathology. Breast Cancer Res Treat. 2005 July; 92(2): 175-86.
Kumar U. Expression of somatostatin receptor subtypes (SSTR1-5) in Alzheimer's disease brain: an immunohistochemical analysis. Neuroscience. 2005; 134(2):525-38.
Liu H L, Huo L, Wang L. Octreotide inhibits proliferation and induces apoptosis of hepatocellular carcinoma cells. Acta Pharmacol Sin. 2004 October; 25(10):1380-6.
Hu C, Yi C, Hao Z, Cao S, Li H, Shao X, Zhang J, Qiao T, Fan D. The effect of somatostatin and SSTR3 on proliferation and apoptosis of gastric cancer cells. Cancer Biol Ther. 2004 August; 3(8):726-30. Epub 2004 Aug. 10.
Goodyer C G, Grigorakis S I, Patel Y C, Kumar U. Developmental changes in the expression of somatostatin receptors (1-5) in the brain, hypothalamus, pituitary and spinal cord of the human fetus. Neuroscience. 2004; 125(2):441-8.
Jia W D, Xu G L, Xu R N, Sun H C, Wang L, Yu J H, Wang J, Li J S, Zhai Z M, Xue Q. Octreotide acts as an antitumor angiogenesis compound and suppresses tumor growth in nude mice bearing human hepatocellular carcinoma xenografts. J Cancer Res Clin Oncol. 2003 June; 129(6):327-34. Epub 2003 Jun. 14.
Vikic-Topic S, Raisch K P, Kvols L K, Vuk-Pavlovic S. Expression of somatostatin receptor subtypes in breast carcinoma, carcinoid tumor, and renal cell carcinoma. J Clin Endocrinol Metab. 1995 October; 80(10):2974-9.
Drebin J A, Hartzell S W, Griffin C, Campbell M J, Niederhuber J E. Molecular cloning and chromosomal localization of the human homologue of a B-lymphocyte specific protein tyrosine kinase (blk). Oncogene. 1995 Feb. 2; 10(3):477-86.
Lin Y H, Shin E J, Campbell M J, Niederhuber J E. Transcription of the blk gene in human B lymphocytes is controlled by two promoters. J Biol Chem. 1995 Oct. 27; 270(43):25968-75.
Law D A, Gold M R, DeFranco A L. Examination of B lymphoid cell lines for membrane immunoglobulin-stimulated tyrosine phosphorylation and src-family tyrosine kinase mRNA expression. Mol Immunol. 1992 July-August; 29(7-8):917-26.
Yao X R, Scott D W. Antisense oligodeoxynucleotides to the blk tyrosine kinase prevent anti-mu-chain-mediated growth inhibition and apoptosis in a B-cell lymphoma. Proc Natl Acad Sci USA. 1993 Sep. 1; 90(17):7946-50.
Vallat L, Magdelenat H, Merle-Beral H, Masdehors P, Potocki de Montalk G, Davi F, Kruhoffer M, Sabatier L, Orntoft T F, Delic J. The resistance of B-CLL cells to DNA damage-induced apoptosis defined by DNA microarrays. Blood. 2003 Jun. 1; 101(11):4598-606. Epub 2003 Feb. 13.
Texido G, Su I H, Mecklenbrauker I, Saijo K, Malek S N, Desiderio S, Rajewsky K, Tarakhovsky A. The B-cell-specific Src-family kinase Blk is dispensable for B-cell development and activation. Mol Cell Biol. 2000 February; 20(4):1227-33.
Malek S N, Dordai D I, Reim J, Dintzis H, Desiderio S. Malignant transformation of early lymphoid progenitors in mice expressing an activated Blk tyrosine kinase. Proc Natl Acad Sci USA. 1998 Jun. 23; 95(13):7351-6.
Goriounov D, Leung C L, Liem R K. Protein products of human Gas2-related genes on chromosomes 17 and 22 (hGAR17 and hGAR22) associate with both microfilaments and microtubules. J Cell Sci. 2003 Mar. 15; 116(Pt 6):1045-58. PMID: 12584248
Brancolini C, Bottega S, Schneider C. Gas2, a growth arrest-specific protein, is a component of the microfilament network system. J Cell Biol. 1992 June; 117(6):1251-61. PMID: 1607387
Stilo R, Liguoro D, Di Jeso B, Formisano S, Consiglio E, Leonardi A, Vito P. Physical and functional interaction of CARMA1 and CARMA3 with Ikappa kinase gamma-NFkappaB essential modulator. J Biol Chem. 2004 Aug. 13; 279(33):34323-31. Epub 2004 Jun. 7.
Rauch A, Devriendt K, Koch A, Rauch R, Gewillig M, Kraus C, Weyand M, Singer H, Reis A, Hofbeck M. Assessment of association between variants and haplotypes of the remaining TBX1 gene and manifestations of congenital heart defects in 22q11.2 deletion patients. J Med Genet. 2004 April; 41(4):e40. No abstract available.
Vitelli F, Taddei I, Morishima M, Meyers E N, Lindsay E A, Baldini A. A genetic link between Tbx1 and fibroblast growth factor signaling. Development. 2002 October; 129(19):4605-11. PMID: 12223416
Zhang L, Zhong T, Wang Y, Jiang Q, Song H, Gui Y. TBX1, a DiGeorge syndrome candidate gene, is inhibited by retinoic acid. Int J Dev Biol. 2006; 50(1):55-61. Nakaya M A, Habas R, Biris K, Dunty W C Jr, Kato Y, He X, Yamaguchi T P. Identification and comparative expression analyses of Daam genes in mouse and Xenopus. Gene Expr Patterns. 2004 November; 5(1):97-105.
Kida Y, Shiraishi T, Ogura T. Identification of chick and mouse Daam1 and Daam2 genes and their expression patterns in the central nervous system. Brain Res Dev Brain Res. 2004 Oct. 15; 153(1):143-50.
Busso D, Cohen D J, Hayashi M, Kasahara M, Cuasnicu P S. Human testicular protein TPX1/CRISP-localization in spermatozoa, fate after capacitation and relevance for gamete interaction. Mol Hum Reprod. 2005 April; 11(4):299-305. Epub 2005 Feb. 25.
Wang H, Zhou Z, Xu M, Li J, Xiao J, Xu Z Y, Sha J. A spermatogenesis-related gene expression profile in human spermatozoa and its potential clinical applications. J Mol Med. 2004 May; 82(5):317-24. Epub 2004 Feb. 24.
Haendler B, Toda I, Sullivan D A, Schleuning W D. Expression of transcripts for cysteine-rich secretory proteins (CRISPs) in the murine lacrimal gland. J Cell Physiol. 1999 March; 178(3):371-8.
Grady R M, Starr D A, Ackerman G L, Sanes J R, Han M. Syne proteins anchor muscle nuclei at the neuromuscular junction. Proc Natl Acad Sci USA. 2005 Mar. 22; 102(12):4359-64. Epub 2005 Mar. 4.
Pare G C, Easlick J L, Mislow J M, McNally E M, Kapiloff M S. Nesprin-1alpha contributes to the targeting of mAKAP to the cardiac myocyte nuclear envelope. Exp Cell Res. 2005 Feb. 15; 303(2):388-99.
Fan J, Beck K A. A role for the spectrin superfamily member Syne-1 and kinesin II in cytokinesis. J Cell Sci. 2004 Feb. 1; 117(Pt 4):619-29. Epub 2004 Jan. 6.
Gough L L, Fan J, Chu S, Winnick S, Beck K A. Golgi localization of Syne-1. Mol Biol Cell. 2003 June; 14(6):2410-24. Epub 2003 Mar. 7.
Zhang Q, Skepper J N, Yang F, Davies J D, Hegyi L, Roberts R G, Weissberg P L, Ellis J A, Shanahan C M. Nesprins: a novel family of spectrin-repeat-containing proteins that localize to the nuclear membrane in multiple tissues. J Cell Sci. 2001 December; 114(Pt 24):4485-98.
Kusano S, Raab-Traub N. I-mfa domain proteins interact with Axin and affect its regulation of the Wnt and c-Jun N-terminal kinase signaling pathways. Mol Cell Biol. 2002 September; 22(18):6393-405.
Tsuji K, Kraut N, Groudine M, Noda M. Vitamin D(3) enhances the expression of 1-mfa, an inhibitor of the MyoD family, in osteoblasts. Biochim Biophys Acta. 2001 May 28; 1539(1-2):122-30. Kraut N, Snider L, Chen C M, Tapscott S J, Groudine M. Requirement of the mouse 1-mfa gene for placental development and skeletal patterning. EMBO J. 1998 Nov. 2; 17(21):6276-88. Hall L, Williams K, Perry A C, Frayne J, Jury J A. The majority of human glutathione peroxidase type 5 (GPX5) transcripts are incorrectly spliced: implications for the role of GPX5 in the male reproductive tract. Biochem J. 1998 Jul. 1; 333 (Pt 1):5-9. PMID: 9639555
Drevet J R, Lareyre J J, Schwaab V, Vernet P, Dufaure J P. The PEA3 protein of the Ets oncogene family is a putative transcriptional modulator of the mouse epididymis-specific glutathione peroxidase gene gpx5. Mol Reprod Dev. 1998 February; 49(2):131-40.
Lareyre J J, Claessens F, Rombauts W, Dufaure J P, Drevet J R. Characterization of an androgen response element within the promoter of the epididymis-specific murine glutathione peroxidase 5 gene. Mol Cell Endocrinol. 1997 Apr. 25; 129(1):33-46.
Karjalainen J M, Kellokoski J K, Mannermaa A J, Kujala H E, Moisio K I, Mitchell P J, Eskelinen M J, Alhava E M, Kosma V M. Failure in post-transcriptional processing is a possible inactivation mechanism of AP-2alpha in cutaneous melanoma. Br J Cancer. 2000 June; 82(12):2015-21. PMID: 10864211
Luscher B, Mitchell P J, Williams T, Tjian R. Regulation of transcription factor AP-2 by the morphogen retinoic acid and by second messengers. Genes Dev. 1989 October; 3(10):1507-17.
Melki S A, Abumrad N A. Expression of the adipocyte fatty acid-binding protein in streptozotocin-diabetes: effects of insulin deficiency and supplementation. J Lipid Res. 1993 September; 34(9): 1527-34.
Turner B C, Zhang J, Gumbs A A, Maher M G, Kaplan L, Carter D, Glazer P M, Hurst H C, Haffly B G, Williams T. Expression of AP-2 transcription factors in human breast cancer correlates with the regulation of multiple growth factor signalling pathways. Cancer Res. 1998 Dec. 1; 58(23):5466-72.
Linton M F, Fazio S. Macrophages, inflammation, and atherosclerosis. Int J Obes Relat Metab Disord. 2003 December; 27 Suppl S35-40. Review.
Jean D, Gershenwald J E, Huang S, Luca M, Hudson M J, Tainsky M A, Bar-Eli M. Loss of AP-2 results in up-regulation of MCAM/MUC18 and an increase in tumor growth and metastasis of human melanoma cells. J Biol Chem. 1998 Jun. 26; 273(26):16501-8. PMID: 9632718
Ropponen K M, Kellokoski J K, Pirinen R T, Moisio K I, Eskelinen M J, Alhava E M, Kosma V M. Expression of transcription factor AP-2 in colorectal adenomas and adenocarcinomas; comparison of immunohistochemistry and in situ hybridization. J Clin Pathol. 2001 July; 54(7):533-8. PMID: 11429425
Zhang L, Zhan S, Navid F, Li Q, Choi Y H, Kim M, Seth P, Helman L J. AP-2 may contribute to IGF-II overexpression in rhabdomyosarcoma. Oncogene. 1998 Sep. 10; 17(10):1261-70. PMID: 9771969
Zhang Q, Rubenstein J N, Jang T L, Pins M, Javonovic B, Yang X, Kim S J, Park I, Lee C. Insensitivity to transforming growth factor-beta results from promoter methylation of cognate receptors in human prostate cancer cells (LNCaP). Mol Endocrinol. 2005 September; 19(9):2390-9. Epub 2005 May 19. PMID: 15905358 [PubMed—in process]
Harder A, Rosche M, Reuss D E, Holtkamp N, Uhlmann K, Friedrich R, Mautner V F, von Deimling A. Methylation analysis of the neurofibromatosis type 1 (NF1) promoter in peripheral nerve sheath tumors. Eur J Cancer. 2004 December; 40(18):2820-8. PMID: 15571966
Zhu C H, Huang Y, Broman M T, Domann F E. Expression of AP-2 alpha in SV40 immortalized human lung fibroblasts is associated with a distinct pattern of cytosine methylation in the AP-2 alpha promoter. Biochim Biophys Acta. 2001 May 28; 1519(1-2):85-91. PMID: 11406275
Sergeeva O A, Amberger B T, Eriksson K S, Scherer A, Haas H L. Co-ordinated expression of 5-HT2C receptors with the NCX1 Na+/Ca2+ exchanger in histaminergic neurones. J Neurochem. 2003 November; 87(3):657-64. PMID: 14535948
Kraev A, Quednau B D, Leach S, Li X F, Dong H, Winkfein R, Perizzolo M, Cai X, Yang R, Philipson K D, Lytton J. Molecular cloning of a third member of the potassium-dependent sodium-calcium exchanger gene family, NCKX3. J Biol Chem. 2001 Jun. 22; 276(25):23161-72. Epub 2001 Apr. 9. PMID: 11294880
Barlund M, Monni O, Weaver J D, Kauraniemi P, Sauter G, Heiskanen M, Kallioniemi O P, Kallioniemi A. Cloning of BCAS3 (17q23) and BCAS4 (20q13) genes that undergo amplification, overexpression, and fusion in breast cancer. Genes Chromosomes Cancer. 2002 December; 35(4):311-7. PMID: 12378525
Feske S, Draeger R, Peter H H, Eichmann K, Rao A. The duration of nuclear residence of NFAT determines the pattern of cytokine expression in human SCID T cells. J Immunol. 2000 Jul. 1; 165(1):297-305. PMID: 10861065
Amasaki Y, Miyatake S, Arai N, Arai K. Regulation of nuclear factor of activated T-cell family transcription factors during T-cell development in the thymus. J Allergy Clin Immunol. 2000 July; 106(1 Pt 2): S1-9. PMID: 10887328
Avni O, Lee D, Macian F, Szabo S J, Glimcher L H, Rao A. T(H) cell differentiation is accompanied by dynamic changes in histone acetylation of cytokine genes. Nat Immunol. 2002 July; 3(7):643-51. Epub 2002 Jun. 10. PMID: 12055628
Willard-Gallo K E, Badran B M, Ravoet M, Zerghe A, Burny A, Martiat P, Goldman M, Roufosse F, Sibille C. Defective CD3gamma gene transcription is associated with NFATc2 overexpression in the lymphocytic variant of hypereosinophilic syndrome. Exp Hematol. 2005 October; 33(10):1147-59. PMID: 16219537
Yang R B, Ng C K, Wasserman S M, Colman S D, Shenoy S, Mehraban F, Komuves L G, Tomlinson J E, Topper J N. Identification of a novel family of cell-surface proteins expressed in human vascular endothelium. J Biol Chem. 2002 Nov. 29; 277(48):46364-73. Epub 2002 Sep. 21. PMID: 12270931
Grimmond S, Larder R, Van Hateren N, Siggers P, Hulsebos T J, Arkell R, Greenfield A. Cloning, mapping, and expression analysis of a gene encoding a novel mammalian EGF-related protein (SCUBE1). Genomics. 2000 Nov. 15; 70(1):74-81. PMID: 11087664
Stadler F, Kolb G, Rubusch L, Baker S P, Jones E G, Akbarian S. Histone methylation at gene promoters is associated with developmental regulation and region-specific expression of ionotropic and metabotropic glutamate receptors in human brain. J Neurochem. 2005 July; 94(2):324-36. PMID: 15998284
Bah J, Quach H, Ebstein R P, Segman R H, Melke J, Jamain S, Rietschel M, Modai I, Kanas K, Karni O, Lerer B, Gourion D, Krebs M O, Etain B, Schurhoff F, Szoke A, Leboyer M, Bourgeron T. Maternal transmission disequilibrium of the glutamate receptor GRIK2 in schizophrenia. Neuroreport. 2004 Aug. 26; 15(12):1987-91. PMID: 15305151
Sinclair P B, Sorour A, Martineau M, Harrison C J, Mitchell W A, O'Neill E, Foroni L. A fluorescence in situ hybridization map of 6q deletions in acute lymphocytic leukemia: identification and analysis of a candidate tumor suppressor gene. Cancer Res. 2004 Jun. 15; 64(12):4089-98. PMID: 15205317
Wolf S, Janzen A, Vekony N, Martine U, Strand D, Closs E I. Expression of solute carrier 7A4 (SLC7A4) in the plasma membrane is not sufficient to mediate amino acid transport activity. Biochem J. 2002 Jun. 15; 364(Pt 3):767-75. PMID: 12049641
Sperandeo M P, Borsani G, Incerti B, Zollo M, Rossi E, Zuffardi O, Castaldo P, Taglialatela M, Andria G, Sebastio G. The gene encoding a cationic amino acid transporter (SLC7A4) maps to the region deleted in the velocardiofacial syndrome. Genomics. 1998 Apr. 15; 49(2):230-6. PMID: 9598310
Peyrl A, Krapfenbauer K, Slavc I, Strobel T, Lubec G. Proteomic characterization of the human cortical neuronal cell line HCN-2. J Chem Neuroanat. 2003 November; 26(3):171-8. PMID: 14615026
Niki T, Takahashi-Niki K, Taira T, Iguchi-Ariga S M, Ariga H. DJBP: a novel DJ-1-binding protein, negatively regulates the androgen receptor by recruiting histone deacetylase complex, and DJ-1 antagonizes this inhibition by abrogation of this complex. Mol Cancer Res. 2003 February; 1(4):247-61. PMID: 12612053
Killebrew D A, Troelstrup D, Shiramizu B. Preferential HIV-1 integration sites in macrophages and HIV-associated malignancies. Cell Mol Biol (Noisy-1e-grand). 2004; 50 Online Pub:OL581-9. PMID: 15555424
Shou S, Scott V, Reed C, Hitzemann R, Stadler H S. Transcriptome analysis of the murine forelimb and hindlimb autopod. Dev Dyn. 2005 September; 234(1):74-89. PMID: 16059910 [PubMed—in process]
Plageman T F Jr, Yutzey K E. T-box genes and heart development: putting the “T” in heart. Dev Dyn. 2005 January; 232(1):11-20. Review. PMID: 15580613
Bussen M, Petry M, Schuster-Gossler K, Leitges M, Gossler A, Kispert A. The T-box transcription factor Tbx18 maintains the separation of anterior and posterior somite compartments. Genes Dev. 2004 May 15; 18(10):1209-21. PMID: 15155583
Rosemann M, Kuosaite V, Nathrath M, Strom T M, Quintanilla-Martinez L, Richter T, Imai K, Atkinson M J. Allelic imbalance at intragenic markers of Tbx18 is a hallmark of murine osteosarcoma. Carcinogenesis. 2003 March; 24(3):371-6. PMID: 12663494
Ding L H, Shingyoji M, Chen F, Hwang J J, Burma S, Lee C, Cheng J F, Chen D J. Gene expression profiles of normal human fibroblasts after exposure to ionizing radiation: a comparative study of low and high doses. Radiat Res. 2005 July; 164(1):17-26. PMID: 15966761
Yankee T M, Draves K E, Clark E A. Expression and function of the adaptor protein Gads in murine B cells. Eur J Immunol. 2005 April; 35(4):1184-92. PMID: 15761845
Shiraiwa H, Takei M, Yoshikawa T, Azuma T, Kato M, Mitamura K, Ueki T, Kida A, Horie T, Seki N, Sawada S. Detection of Grb-2-related adaptor protein gene (GRAP) and peptide molecule in salivary glands of MRL/lpr mice and patients with Sjogren's syndrome. J Int Med Res. 2004 May-June; 32(3):284-91. PMID: 15174222
Guyot B, Mouchiroud G. Characterization of promoter elements directing Mona/Gads molecular adapter expression in T and myelomonocytic cells: involvement of the AML-1 transcription factor. J Leukoc Biol. 2003 February; 73(2):263-72. PMID: 12554803
Guyot B, Arnaud S, Phothirath P, Bourette R P, Grasset M F, Rigal D, Mouchiroud G. Genomic organization and restricted expression of the human Mona/Gads gene suggests regulation by two specific promoters. Gene. 2002 May 15; 290(1-2):173-9. PMID: 12062812
Chou H H, Hayakawa T, Diaz S, Krings M, Indriati E, Leakey M, Paabo S, Satta Y, Takahata N, Varki A. Inactivation of CMP-N-acetylneuraminic acid hydroxylase occurred prior to brain expansion during human evolution. Proc Natl Acad Sci USA. 2002 Sep. 3; 99(18):11736-41. Epub 2002 Aug. 21. PMID: 12192086
Igaz P, Salvi R, Rey J P, Glauser M, Pralong F P, Gaillard R C. Effects of cytokines on GnRH gene expression in primary hypothalamic neurons and in GnRH neurons immortalized conditionally. Endocrinology. 2005 Nov. 10; [Epub ahead of print] PMID: 16282355 [PubMed—as supplied by publisher]
Loewen G M, Tracy E, Blanchard F, Tan D, Yu J, Raza S, Matsui S, Baumann H. Transformation of human bronchial epithelial cells alters responsiveness to inflammatory cytokines. BMC Cancer. 2005 Nov. 4; 145. PMID: 16271139 [PubMed—in process]
Znoyko I, Sohara N, Spicer S S, Trojanowska M, Reuben A. Expression of oncostatin M and its receptors in normal and cirrhotic human liver. J Hepatol. 2005 November; 43(5):893-900. Epub 2005 Jul. 26. PMID: 16169119 [PubMed—in process]
Finelt N, Gazel A, Gorelick S, Blumenberg M. Transcriptional responses of human epidermal keratinocytes to Oncostatin-M. Cytokine. 2005 Aug. 21; 31(4):305-13. PMID: 16023359
Kong W, Abidi P, Kraemer F B, Jiang J D, Liu J. In vivo activities of cytokine oncostatin M in the regulation of plasma lipid levels. J Lipid Res. 2005 June; 46(6):1163-71. Epub 2005 Mar. 16. PMID: 15772430
Znoyko I, Sohara N, Spicer S S, Trojanowska M, Reuben A. Comparative studies of oncostatin M expression in the tissues of adult rodents. Anat Rec A Discov Mol Cell Evol Biol. 2005 March; 283(1):182-6. PMID: 15674824
Li C, Zhang F, Lin M, Liu J. Induction of S100A9 gene expression by cytokine oncostatin M in breast cancer cells through the STAT3 signaling cascade. Breast Cancer Res Treat. 2004 September; 87(2):123-34. PMID: 15377837
Holzer R G, Ryan R E, Tommack M, Schlekeway E, Jorcyk C L. Oncostatin M stimulates the detachment of a reservoir of invasive mammary carcinoma cells: role of cyclooxygenase-2. Clin Exp Metastasis. 2004; 21(2):167-76. PMID: 15168734
Cross A, Edwards S W, Bucknall R C, Moots R J. Secretion of oncostatin M by neutrophils in rheumatoid arthritis. Arthritis Rheum. 2004 May; 50(5):1430-6. PMID: 15146412
Gwechenberger M, Moertl D, Pacher R, Huelsmann M. Oncostatin-M in myocardial ischemia/reperfusion injury may regulate tissue repair. Croat Med J. 2004 April; 45(2):149-57. PMID: 15103750
Levy M T, Trojanowska M, Reuben A. Oncostatin M: a cytokine upregulated in human cirrhosis, increases collagen production by human hepatic stellate cells. J Hepatol. 2000 February; 32(2):218-26. PMID: 10707861
Vasse M, Pourtau J, Trochon V, Muraine M, Vannier J P, Lu H, Soria J, Soria C. Oncostatin M induces angiogenesis in vitro and in vivo. Arterioscler Thromb Vasc Biol. 1999 August; 19(8):1835-42. PMID: 10446061
Battini R, Battaglia A, Bertini V, Cioni G, Parrini B, Rapalini E, Simi P, Tinelli F, Valetto A. Characterization of the phenotype and definition of the deletion in a new patient with ring chromosome 22. Am J Med Genet A. 2004 Oct. 1; 130(2):196-9. PMID: 15372517
Formstone C J, Mason I. Expression of the Celsr/flamingo homologue, c-fmi1, in the early avian embryo indicates a conserved role in neural tube closure and additional roles in asymmetry and somitogenesis. Dev Dyn. 2005 February; 232(2):408-13. PMID: 15614764
Tissir F, De-Backer O, Goffinet A M, Lambert de Rouvroit C. Developmental expression profiles of Celsr (Flamingo) genes in the mouse. Mech Dev. 2002 March; 112(1-2):157-60. PMID: 11850187
Bacon L, Eagle R A, Meyer M, Easom N, Young N T, Trowsdale J. Two human ULBP/RAET1 molecules with transmembrane regions are ligands for NKG2D. J Immunol. 2004 Jul. 15; 173(2):1078-84. PMID: 15240696
Jan Chalupny N, Sutherland C L, Lawrence W A, Rein-Weston A, Cosman D. ULBP4 is a novel ligand for human NKG2D. Biochem Biophys Res Commun. 2003 May 23; 305(1):129-35. PMID: 12732206
Eckfeld K, Hesson L, Vos M D, Bieche I, Latif F, Clark G J. RASSF4/AD037 is a potential ras effector/tumor suppressor of the RASSF family. Cancer Res. 2004 Dec. 1; 64(23):8688-93. PMID: 15574778
Chow L S, Lo K W, Kwong J, Wong A Y, Huang D P. Aberrant methylation of RASSF4/AD037 in nasopharyngeal carcinoma. Oncol Rep. 2004 October; 12(4):781-7. PMID: 15375500
Futscher B W, Oshiro M M, Wozniak R J, Holtan N, Hanigan C L, Duan H, Domann F E. (2002) Role for DNA methylation in the control of cell type specific maspin expression. Nat Genet. 31(2):175-9.

1. A method for classifying a biological sample, comprising:
obtaining a biological sample from a subject;
determining the expression status of at least one gene or genomic sequence selected from the group consisting the genes or genomic sequences according to Table 1 in said sample; and
classifying said biological sample according to said expression status.

2. The method according to claim 1, wherein the class is selected from the group consisting of organ type, tissue type, cell type and disease state.

3. The method according to claim 2, wherein said class consists of T-lymphocytes.

4. The method according to claim 3, wherein said genomic sequences are selected from at least one of Tables 8A and 9A.

5. The method according to claim 2, wherein said class consists of embryonic liver.

6. The method according to claim 5, wherein said genomic sequences are selected from at least one of Tables 8B and 9B.

7. The method according to claim 2, wherein said class consists of embryonic skeletal muscle.

8. The method according to claim 7, wherein said genomic sequences are selected from at least one of Tables 8C and 9C.

9. The method according to claim 2, wherein said class consists of fibroblasts.

10. The method according to claim 9, wherein said genomic sequences are selected from at least one of Tables 8D and 9D.

11. The method according to claim 2, wherein said class consists of heart muscle.

12. The method according to claim 11, wherein said genomic sequences are selected from at least one of Tables 8E and 9E.

13. The method according to claim 2, wherein said classes are heart muscle and skeletal muscle.

14. The method according to claim 13, wherein said genomic sequences are selected from Table 8F.

15. The method according to claim 2, wherein said class consists of keratinocytes.

16. The method according to claim 15, wherein said genomic sequences are selected from at least one of Tables 8G and 9F.

17. The method according to claim 2, wherein said class consists of liver.

18. The method according to claim 17, wherein said genomic sequences are selected from at least one of Tables 8H and 9G.

19. The method according to claim 2, wherein said class consists of melanocytes.

20. The method according to claim 19, wherein said genomic sequences are selected from at least one of Table 8I and 9H.

21. The method according to claim 2, wherein said class consists of placenta.

22. The method according to claim 21, wherein said genomic sequences are selected from at least one of Table 8J and 9I.

23. The method according to claim 2, wherein said class consists of skeletal muscle.

24. The method according to claim 23, wherein said genomic sequences are selected from at least one of Table 8K and 9J.

25. The method according to claim 2, wherein said class consists of sperm.

26. The method according to claim 25, wherein said genomic sequences are selected from Table 8L.

27. The method according to claim 1, wherein the sample is selected from the group consisting of cells or cell lines, histological slides, biopsies, paraffin-embedded tissue, bodily fluids, sputum, stool, nipple aspirate, cerebrospinal fluid, ejaculate, urine, blood, and combinations thereof.

28. The method according to claim 1, wherein the expression is determined by measuring the level of at least one of mRNA, cDNA or polypeptide.

29. The method according to claim 1, wherein the expression is determined by use of at least one technique selected from the group of Northern blot analysis, reverse transcriptase PCR, real-time PCR, RNAse protection, and microarray.

30. The method according to claim 1, wherein said expression is determined by determining the level of methylation or methylation status of one or more CpG positions within said genes or genomic regions.

31. The method according to claim 30, comprising contacting genomic DNA isolated from a biological sample, with at least one reagent, or series of reagents that distinguishes between methylated and non-methylated CpG dinucleotides within at least one target region of the genomic DNA, wherein the target region comprises, or hybridizes under stringent conditions to a sequence of at least 16 contiguous nucleotides of at least one gene or genomic sequence selected from Table 1, wherein said contiguous nucleotides comprise at least one CpG dinucleotide sequence, and wherein classification of said sample is, at least in part, afforded.

32. The method according to claim 31, comprising:
isolating genomic DNA from a biological sample taken from a subject;
treating the genomic DNA, or a portion or fragment thereof, with one or more reagents to convert 5-position unmethylated cytosine bases to uracil or to another base that is detectably dissimilar to cytosine in terms of hybridization properties;
contacting the treated genomic DNA, or the treated portion or fragment thereof, with an amplification enzyme and at least two primers comprising, in each case a contiguous sequence at least 18 nucleotides in length that is complementary to, or hybridizes under moderately stringent or stringent conditions to a converted sequence selected from Table 1 and complements thereof, wherein the treated DNA or a fragment thereof is either amplified to produce one or more amplificates, or is not amplified;
determining, based on the presence or absence of, or on the quantity or on a property of said amplificate, the methylation state of at least one CpG dinucleotide sequence of at least one gene or sequence selected from Table 1, or an average, or a value reflecting an average methylation state of a plurality of CpG dinucleotide sequences of at least one gene or sequence selected from Table 1; and
classifying said sample according to said methylation state.

33. A treated nucleic acid derived from the genomic sequences of Table 2, wherein the treatment is suitable to convert at least one unmethylated cytosine base of the genomic DNA sequence to uracil or another base that is detectably dissimilar to cytosine in terms of hybridization.

34. A nucleic acid, comprising at least 16 contiguous nucleotides of a treated genomic DNA sequence selected from the converted sequences according to Table 1 and sequences complementary thereto, wherein said nucleic acid is not identical or complementary to a genomic sequence according to Table 2, and wherein the treatment is suitable to convert at least one unmethylated cytosine base of the genomic DNA sequence to uracil or another base that is detectably dissimilar to cytosine in terms of hybridization.

35. The nucleic acid of any one of claims 33 and 34, wherein the contiguous base sequence comprises at least one CpG, TpG or CpA dinucleotide sequence.

36. The nucleic acid of claim 35, wherein the treatment comprises use of a reagent selected from the group consisting of bisulfite, hydrogen sulfite, disulfite, and combinations thereof.

37. An oligomer, comprising a sequence of at least 9 contiguous nucleotides that is complementary to, or hybridizes under moderately stringent or stringent conditions to a treated genomic DNA sequence selected from the converted sequences according to Table 2 and sequences complementary thereto, wherein said nucleic acid is not identical or complementary to a genomic sequence according to Table 2.

38. The oligomer of claim 37, comprising at least one CpG, CpA or TpG dinucleotide sequence.

39. A kit for use in for use in the classification of a biological sample, the kit comprising means for detecting the polypeptides of a gene or genomic region selected from Table 1.

40. The kit according to claim 39, further comprising:
a container suitable for containing the means for detecting polypeptides, and a biological sample of the patient comprising said polypeptides, wherein the means for detecting polypeptides can from complexes with the polypeptides; and
a means to detect the complexes.

41. A kit for use in the classification of a biological sample, comprising means for measuring the level of mRNA transcription of a gene or genomic region selected from Table 1.

42. The kit according to claim 41, further comprising:
a container suitable for containing the means for measuring the level of mRNA, and a biological sample of the patient comprising mRNA of a gene or genomic region selected from Table 1 wherein the means for measuring the level of mRNA are able to hybridize to the mRNA; and
a means for detecting the hybridized mRNA complexes.

43. A kit comprising:
a) at least one bisulfite reagent selected from the group consisting of bisulfite hydrogen sulfite, and disulfite; and
b) at least two nucleic acid molecules comprising, in each case a contiguous sequence at least 16 nucleotides that is complementary to, or hybridizes under moderately stringent or stringent conditions to a converted sequence selected from Table 2, and complements thereof.

44. A composition comprising the following:
a nucleic acid comprising a sequence at least 18 bases in length of a segment of the converted genomic DNA according to one of the converted sequences of Table 2 and sequences complementary thereto; and
a buffer comprising at least one of magnesium chloride, dNTP, taq polymerase, an oligomer, in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a converted genomic DNA according to one of the converted sequences of Table 1 and sequences complementary thereto.

45. (canceled)

46. The kit of claim 40, further comprising instructions for use and interpretation of the kit results.

47. The kit of claim 42, further comprising instructions for use and interpretation of the kit results.
